(19) United States
(12) Patent Application Publication
(10) Pub. No.: US 2024/0287087 A1
(43) Pub. Date: Aug. 29, 2024

(54) COMPOUND AND ORGANIC LIGHT EMITTING DEVICE COMPRISING THE SAME

(71) Applicant: LG CHEM, LTD., Seoul (KR)

(72) Inventors: Minjun KIM, Daejeon (KR); Dong Hoon LEE, Daejeon (KR); Sang Duk SUH, Daejeon (KR); Young Seok KIM, Daejeon (KR)

(21) Appl. No.: 18/289,222

(22) PCT Filed: Oct. 14, 2022
(86) PCT No.: PCT/KR2022/015547

§ 371 Date: Nov. 1, 2023

(30) Foreign Application Priority Data
Oct. 14, 2021 (KR) 10-2021-0136430
Oct. 13, 2022 (KR) 10-2022-0131323

(51) Int. Cl. C07D 491/048; H10K 50/12; H10K 85/10

(52) U.S. Cl. CPC C07D 491/048; H10K 85/111; H10K 50/121

(57) ABSTRACT

A compound represented by Chemical Formula I and an organic light emitting device including the same are provided.

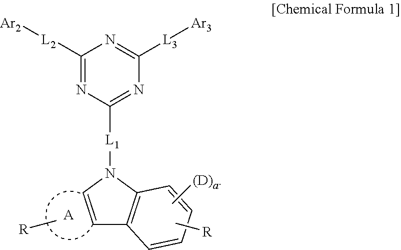

### CROSS-REFERENCE TO RELATED APPLICATION(S)

This application is a National Phase entry pursuant to 35 U.S.C. § 371 of International Application No. PCT/KR2022/015547 filed on Oct. 14, 2022, and claims priority to and the benefit of Korean Patent Application No. 10-2021-0136430 filed on Oct. 14, 2021 and Korean Patent Application No. 10-2022-0131323 filed on Oct. 13, 2022, the disclosures of which are incorporated herein by reference in their entireties.

### FIELD OF DISCLOSURE

The present disclosure relates to a novel compound and an organic light emitting device comprising the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuous need to develop a new material for the organic material used in the organic light emitting device as described above.

### RELATED ART

Korean Unexamined Patent Publication No. 10-2000-0051826

### SUMMARY

It is an object of the present disclosure to provide a novel compound and an organic light emitting device comprising the same.

According to an aspect of the present disclosure, there is provided a compound represented by the following Chemical Formula 1:

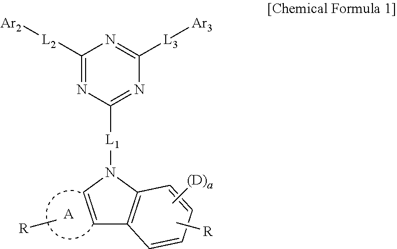

in Chemical Formula 1,
A is a substituted or unsubstituted benzene or naphthalene,
each R is independently hydrogen, deuterium, a substituted or unsubstituted C₆₋₆₀ aryl, or a substituted or unsubstituted C₂₋₆₀ heteroaryl containing one or more selected from the group consisting of N, O and S,
D is deuterium,
a is an integer of 0 to 3,
L₁ is a single bond, phenylene, biphenyldiyl, naphthalenediyl, (phenylene)(naphthalenediyl), or (naphthalenediyl)(phenylene), (naphthalenediyl)(naphthalenediyl),
wherein, when L₁ is phenylene, biphenyldiyl, naphthalenediyl, (naphthalenediyl)(phenylene), (phenylene)(naphthalenediyl), or (naphthalenediyl)(naphthalenediyl), L₁ is unsubstituted or substituted with at least one deuterium,
L₂ and L₃ are each independently a single bond, a substituted or unsubstituted C₆₋₆₀ arylene, or a substituted or unsubstituted C₂₋₆₀ heteroarylene containing one or more selected from the group consisting of N, O and S,
Ar₂ is a substituted or unsubstituted C₆₋₆₀ aryl, or a substituted or unsubstituted C₂₋₆₀ heteroaryl containing one or more selected from the group consisting of N, O and S, and
Ar₃ is a substituent represented by the following Chemical Formula 2,

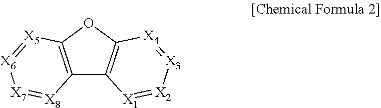

in Chemical Formula 2,
one of X₁ to X₈ is N, another one is C bonded to L₃, and the rest is CH or CD,
provided that when X₁ is N, one of X₅ to X₈ is C bonded to L₃, and when X₈ is N, one of X₁ to X₄ is C bonded to L₃.

According to another aspect of the present disclosure, there is provided an organic light emitting device comprising: a first electrode; a second electrode provided to face the first electrode; and an organic material layer including one or more layers provided between the first electrode and the second electrode, wherein one or more layers of the organic material layer comprise the compound represented by Chemical Formula 1.

The above-mentioned compound represented by Chemical Formula 1 can be used as a material of an organic material layer in an organic light emitting device, and can improve the efficiency, achieve low driving voltage and/or improve lifetime characteristics in the organic light emitting device. In particular, the compound represented by the Chemical Formula 1 can be used as a hole injection material, hole transport material, hole injection and transport material, light emitting material, electron transport material, or electron injection material.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, a second hole transport layer 7, a light emitting layer 3, a hole blocking layer 8, an electron injection and transport layer 9, and a cathode 4.

### DETAILED DESCRIPTION

Hereinafter, embodiments of the present disclosure will be described in more detail to facilitate understanding of the invention.

As used herein, the notation [symbol] and [symbol] mean a bond linked to another substituent group.

As used herein, the term “substituted or unsubstituted” means being unsubstituted or substituted with one or more substituents selected from the substituent group consisting of deuterium; a halogen group; a nitrile group; a nitro group; a hydroxy group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heterocyclic group containing at least one of N, O and S atoms, or being unsubstituted or substituted with a substituent from the above substituent group which is further substituted by one or more selected from the above substituent group.

In the present disclosure, the carbon number of a carbonyl group is not particularly limited, but is preferably 1 to 40. Specifically, it may be a substituent having the following structure, but is not limited thereto.

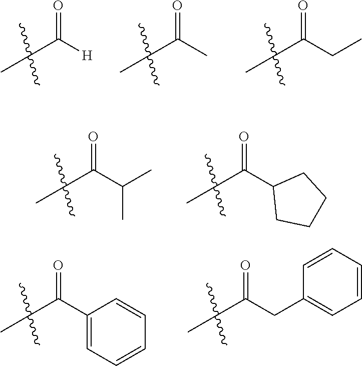

In the present disclosure, an ester group may have a structure in which oxygen of the ester group may be substituted by a straight-chain, branched-chain, or cyclic alkyl group having 1 to 25 carbon atoms, or an aryl group having 6 to 25 carbon atoms. Specifically, it may be a substituent having the following structure, but is not limited thereto.

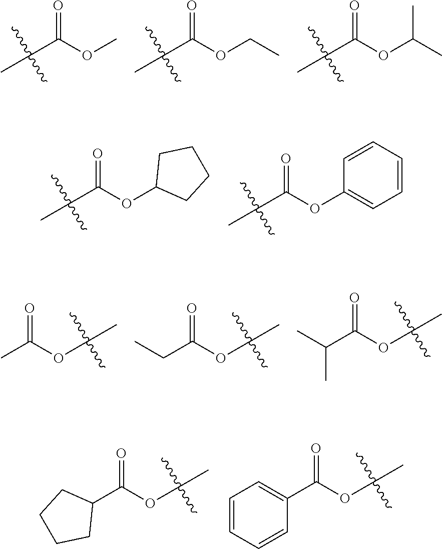

In the present disclosure, the carbon number of an imide group is not particularly limited, but is preferably 1 to 25. Specifically, it may be a substituent having the following structure, but is not limited thereto.

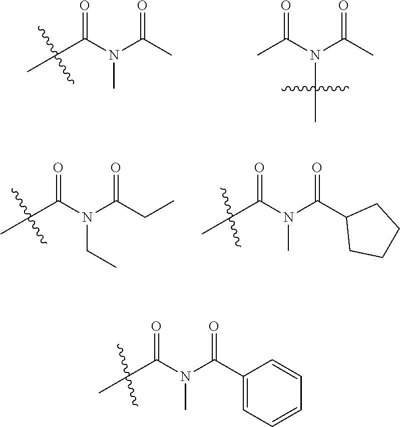

In the present disclosure, a silyl group specifically includes trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but is not limited thereto.

In the present disclosure, examples of a halogen group include fluorine, chlorine, bromine, or iodine.

In the present disclosure, the alkyl group may be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclohectylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group may be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl) vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it may be a monocyclic aryl group or a polycyclic aryl group. According to one embodiment, the carbon number of the aryl group is 6 to 30. According to one embodiment, the carbon number of the aryl group is 6 to 20. The aryl group may be a phenyl group, a biphenylyl group, a terphenylyl group or the like as the monocyclic aryl group, but is not limited thereto. The polycyclic aryl group includes a naphthyl group, an anthracenyl group, a phenanthryl group, a pyrenyl group, a perylenyl group, a chrysenyl group, a fluorenyl group, or the like, but is not limited thereto.

In the present disclosure, the fluorenyl group may be substituted, and two substituents may be linked with each other to form a spiro structure. In the case where the fluorenyl group is substituted,

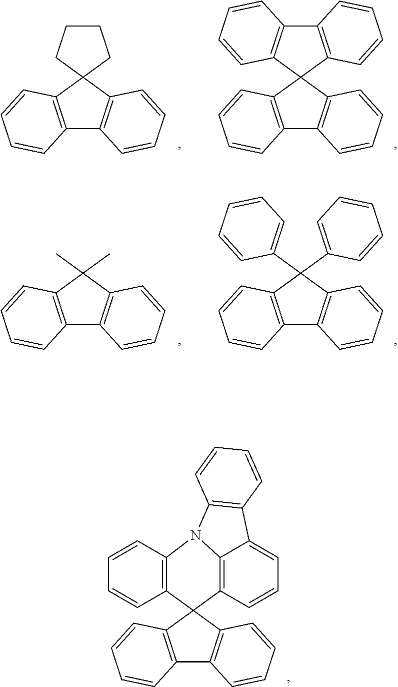

and the like can be formed.

In the present disclosure, a heterocyclic group is a heterocyclic group containing at least one of O, N, Si and S as a heteroatom, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, an aromatic ring means a condensed monocyclic or condensed polycyclic ring in which the entire molecule has aromaticity while containing only carbon as a ring-forming atom. The carbon number of the aromatic ring is 6 to 60, or 6 to 30, or 6 to 20, but is not limited thereto. In addition, the aromatic ring may include a benzene ring, a naphthalene ring, an anthracene ring, a phenanthrene ring, a pyrene ring, and the like, but is not limited thereto.

In the present disclosure, the aryl group in the aralkyl group, the aralkenyl group, the alkylaryl group and the arylamine group is the same as the above-mentioned examples of the aryl group. In the present disclosure, the alkyl group in the aralkyl group, the alkylaryl group and the alkylamine group is the same as the above-mentioned examples of the alkyl group. In the present disclosure, the heteroaryl in the heteroarylamine may be applied to the above-mentioned description of the heterocyclic group. In the present disclosure, the alkenyl group in the aralkenyl group is the same as the above-mentioned examples of the alkenyl group. In the present disclosure, the above-mentioned description of the aryl group may be applied except that the arylene is a divalent group. In the present disclosure, the above-mentioned description of the heterocyclic group can be applied except that the heteroarylene is a divalent group. In the present disclosure, the above-mentioned description of the aryl group or cycloalkyl group may be applied except that the hydrocarbon ring is not a monovalent group but formed by combining two substituent groups. In the present disclosure, the above-mentioned description of the heterocyclic group may be applied except that the heterocyclic group is not a monovalent group but formed by combining two substituent groups.

In Chemical Formula 1, at least one hydrogen may be substituted with deuterium.

Preferably, A is a benzene unsubstituted or substituted with 1 to 4 deuterium; or naphthalene unsubstituted or substituted with 1 to 6 deuterium.

Depending on the fusion position of A in Chemical Formula 1, the compound of Chemical Formula 1 may be represented by one of the following Chemical Formulas 1-1 to 1-4:

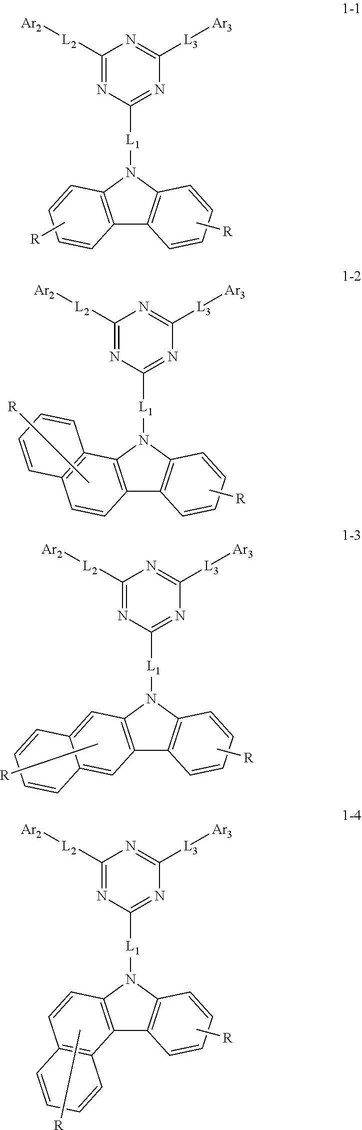

in Chemical Formulas 1-1 to 1-4,
R, L₁, L₂, L₃, Ar₂ and Ar₃ are the same as defined above.

Preferably, each R is independently hydrogen, deuterium, phenyl, biphenylyl, naphthyl, dibenzofuranyl, dibenzothiophenyl, 9H-carbazol-9-yl, or 9-phenyl-9H-carbazolyl, each of which, except for hydrogen and deuterium, is unsubstituted or substituted with at least one deuterium.

More preferably, each R is hydrogen; or each R is deuterium; or one R is hydrogen or deuterium, and the other R is phenyl, biphenylyl, naphthyl, dibenzofuranyl, dibenzothiophenyl, 9H-carbazol-9-yl, or 9-phenyl-9H-carbazolyl, each of which is unsubstituted or substituted with at least one deuterium.

Preferably, L₁ is a single bond, phenylene, biphenyldiyl, or naphthalenediyl, each of which, except for a single bond, is unsubstituted or substituted with at least one deuterium.

Preferably, L₂ and L₃ are each independently a single bond, phenylene, biphenyldiyl, or naphthylene, each of which, except for a single bond, is unsubstituted or substituted with at least one deuterium.

Preferably, L₃ is a single bond, phenylene, or naphthylene, each of which, except for a single bond, is unsubstituted or substituted with at least one deuterium.

Preferably, Ar₂ is phenyl, biphenylyl, terphenylyl, naphthyl, phenylnaphthyl, naphthylphenyl, phenanthrenyl, dimethylfluorenyl, diphenylfluorenyl, dibenzofuranyl, dibenzothiophenyl, 9H-carbazol-9-yl, or 9-phenyl-9H-carbazolyl, each of which is unsubstituted or substituted with at least one deuterium or triphenylsilyl.

More preferably, Ar₂ is phenyl, (triphenylsilyl)phenyl, biphenylyl, terphenylyl, naphthyl, phenylnaphthyl, naphthylphenyl, phenanthrenyl, dimethylfluorenyl, diphenylfluorenyl, dibenzofuranyl, dibenzothiophenyl, 9H-carbazol-9-yl, or 9-phenyl-9H-carbazolyl, each of which is unsubstituted or substituted with at least one deuterium.

Preferably, the substituent represented by Chemical Formula 2 is represented by any one of the following Chemical Formulas 2a to 2d:

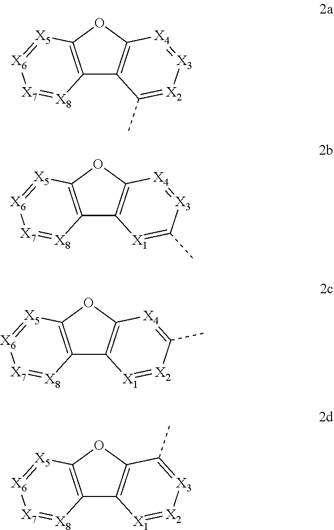

in Chemical Formula 2a, one of X₂ to X₄ and X₅ to X₅ is N, and the rest are each independently CH or CD,
in Chemical Formula 2b, one of X₁ and X₃ to X₈ is N, and the rest are each independently CH or CD,
in Chemical Formula 2c, one of X₁, X₂ and X₄ to X₈ is N, and the rest are each independently CH or CD, and
in Chemical Formula 2d, one of X₁ to X₃ and X₅ to X₈ is N, and the rest are each independently CH or CD.

More preferably, in Chemical Formula 2, if X₁ is N, one of X₅ to X₈ is C bonded to L₃, and thus,
in Chemical Formula 2a, one of X₂ to X₄ and X₅ to X₈ is N, and the rest are each independently CH or CD,
in Chemical Formula 2b, X₁ is CH or CD; one of X₃ to X₈ is N, and the rest are each independently CH or CD,
in Chemical Formula 2c, X₁ is CH or CD; one of X₂ and X₄ to X₈ is N, and the rest are each independently CH or CD, and
in Chemical Formula 2d, X₁ is CH or CD; one of X₂, X₃ and X₅ to X₈ is N, and the rest are each independently CH or CD.

Representative examples of the compound represented by Chemical Formula 1 are as follows:

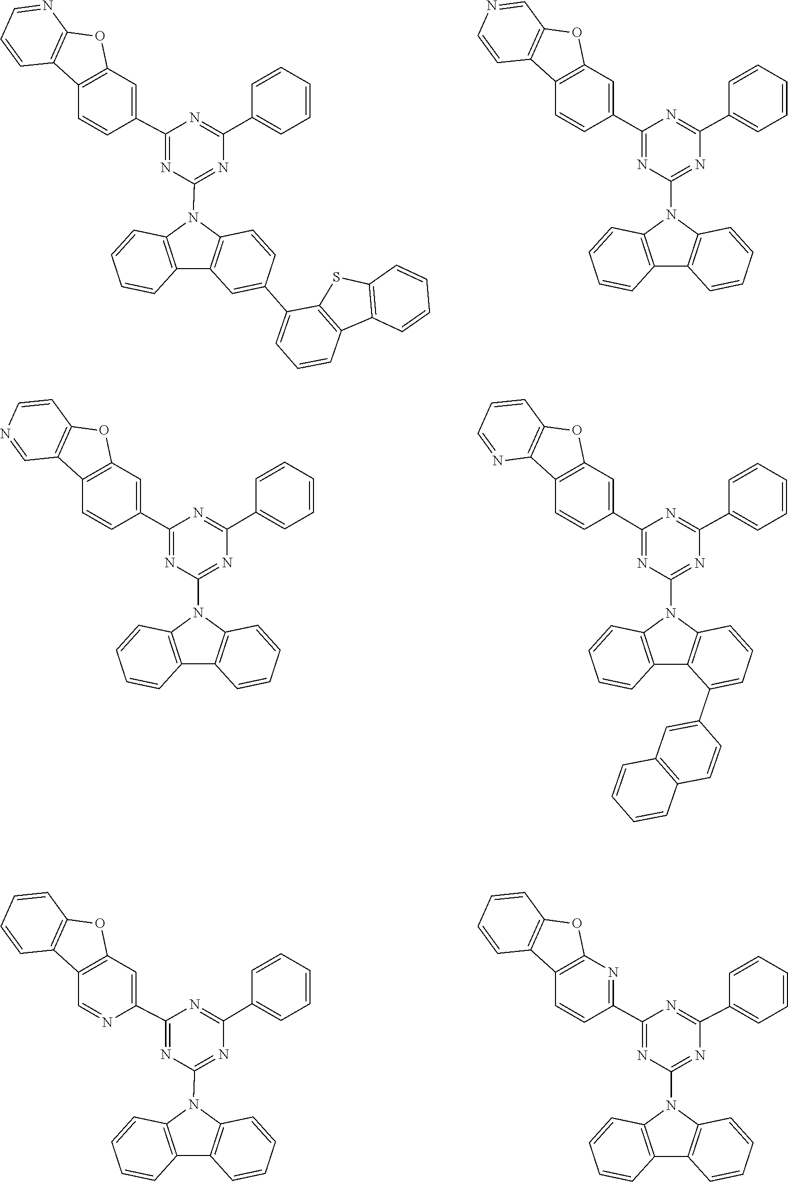 [SMILES: c1ccc(-c2nc(-c3ccc4c(c3)oc3ncccc34)nc(-n3c4ccccc4c4cc(-c5cccc6c5sc5ccccc56)ccc43)n2)cc1 ; c1ccc(-c2nc(-c3ccc4c(c3)oc3cnccc34)nc(-n3c4ccccc4c4ccccc43)n2)cc1 ; c1ccc(-c2nc(-c3ccc4c(c3)oc3ccncc34)nc(-n3c4ccccc4c4ccccc43)n2)cc1 ; c1ccc(-c2nc(-c3ccc4c(c3)oc3cccnc34)nc(-n3c4ccccc4c4c(-c5ccc6ccccc6c5)cccc43)n2)cc1 ; c1ccc(-c2nc(-c3cc4oc5ccccc5c4cn3)nc(-n3c4ccccc4c4ccccc43)n2)cc1 ; c1ccc(-c2nc(-c3ccc4c(n3)oc3ccccc34)nc(-n3c4ccccc4c4ccccc43)n2)cc1]

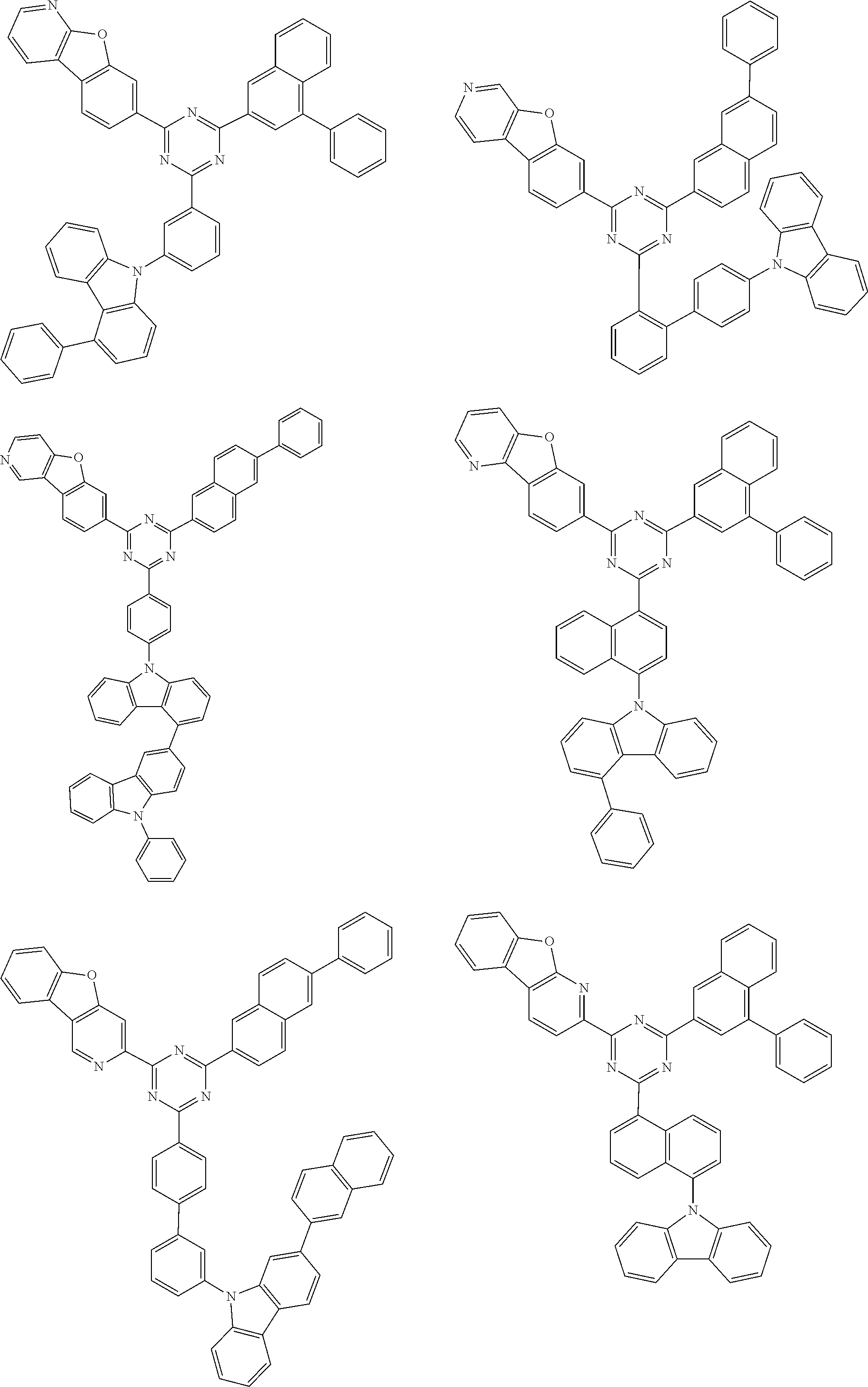

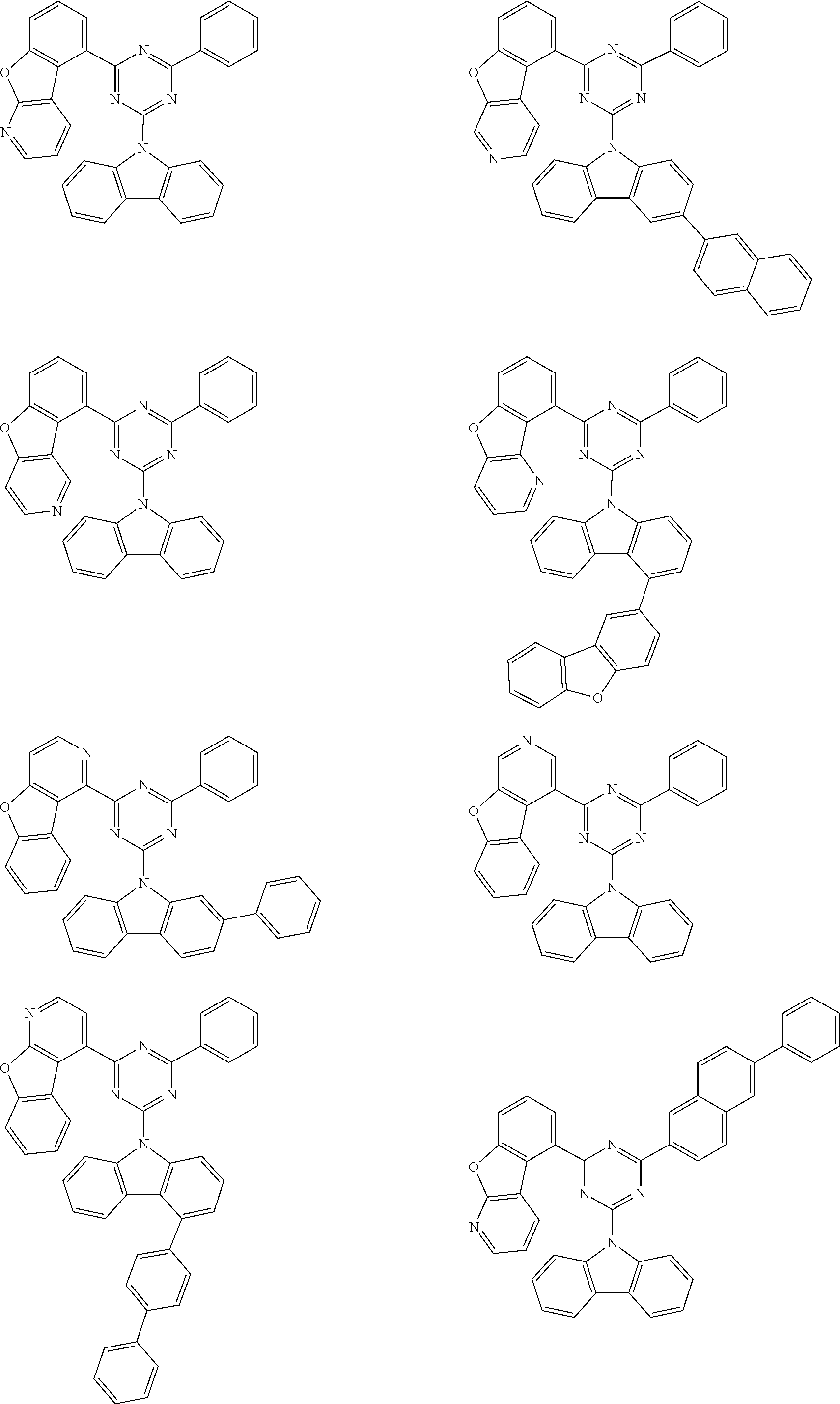

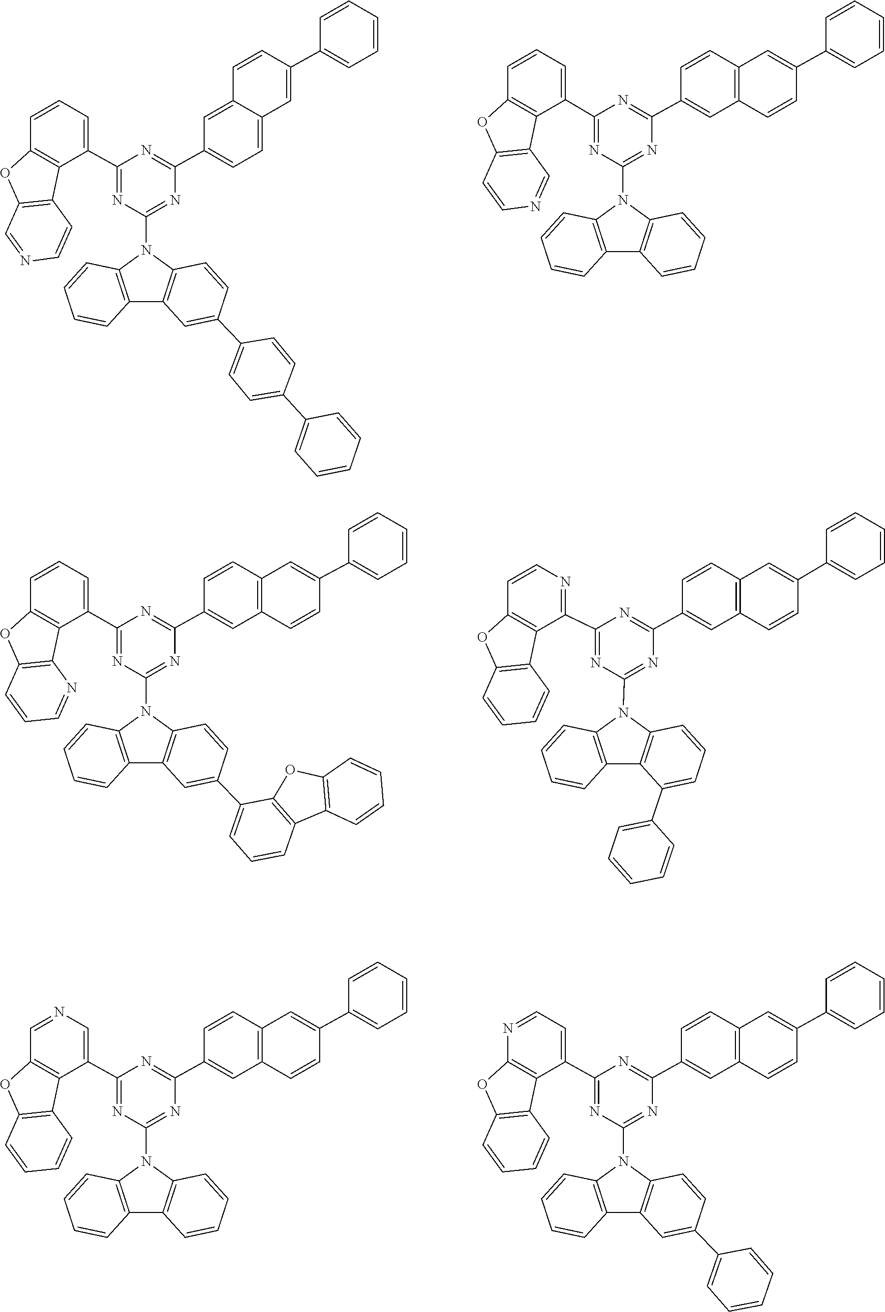

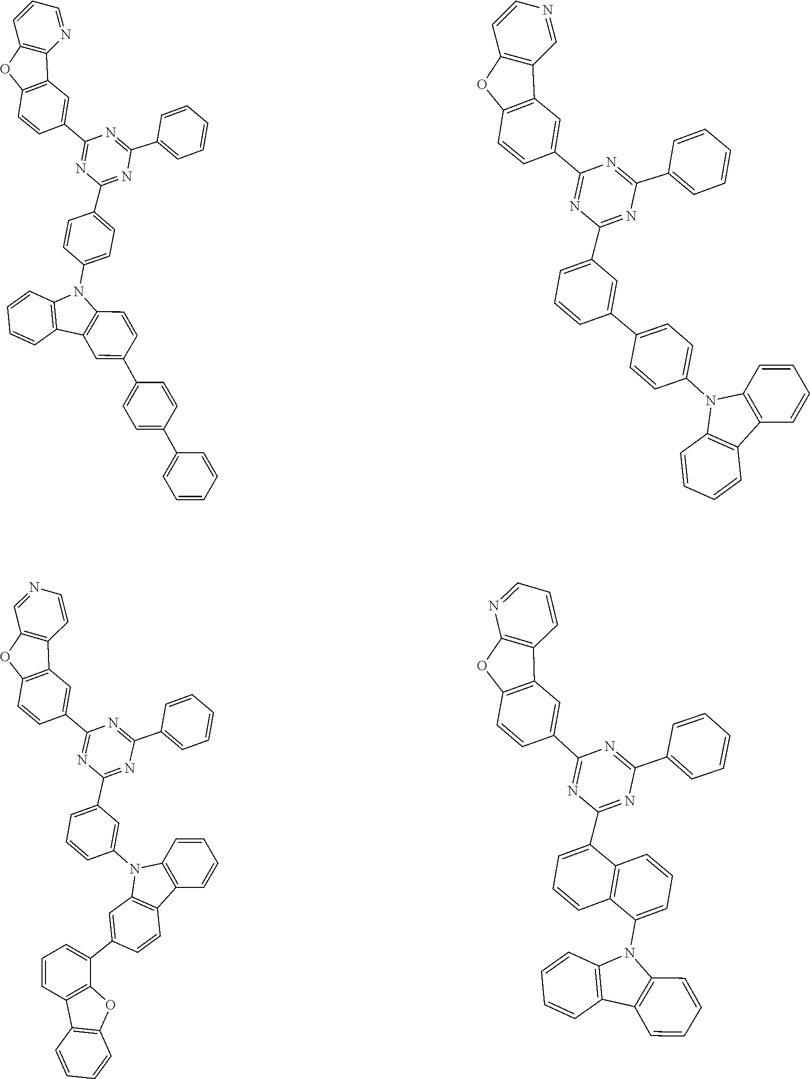 [SMILES: c1ccc(-c2ccc(-c3ccc4c(c3)c3ccccc3n4-c3ccc(-c4nc(-c5ccccc5)nc(-c5ccc6oc7cccnc7c6c5)n4)cc3)cc2)cc1 ; c1ccc(-c2nc(-c3cccc(-c4ccc(-n5c6ccccc6c6ccccc65)cc4)c3)nc(-c3ccc4oc5ccncc5c4c3)n2)cc1 ; c1ccc(-c2nc(-c3cccc(-n4c5ccccc5c5ccc(-c6cccc7c6oc6ccccc67)cc54)c3)nc(-c3ccc4oc5cnccc5c4c3)n2)cc1 ; c1ccc(-c2nc(-c3ccc4oc5ncccc5c4c3)nc(-c3cccc4c(-n5c6ccccc6c6ccccc65)cccc34)n2)cc1]

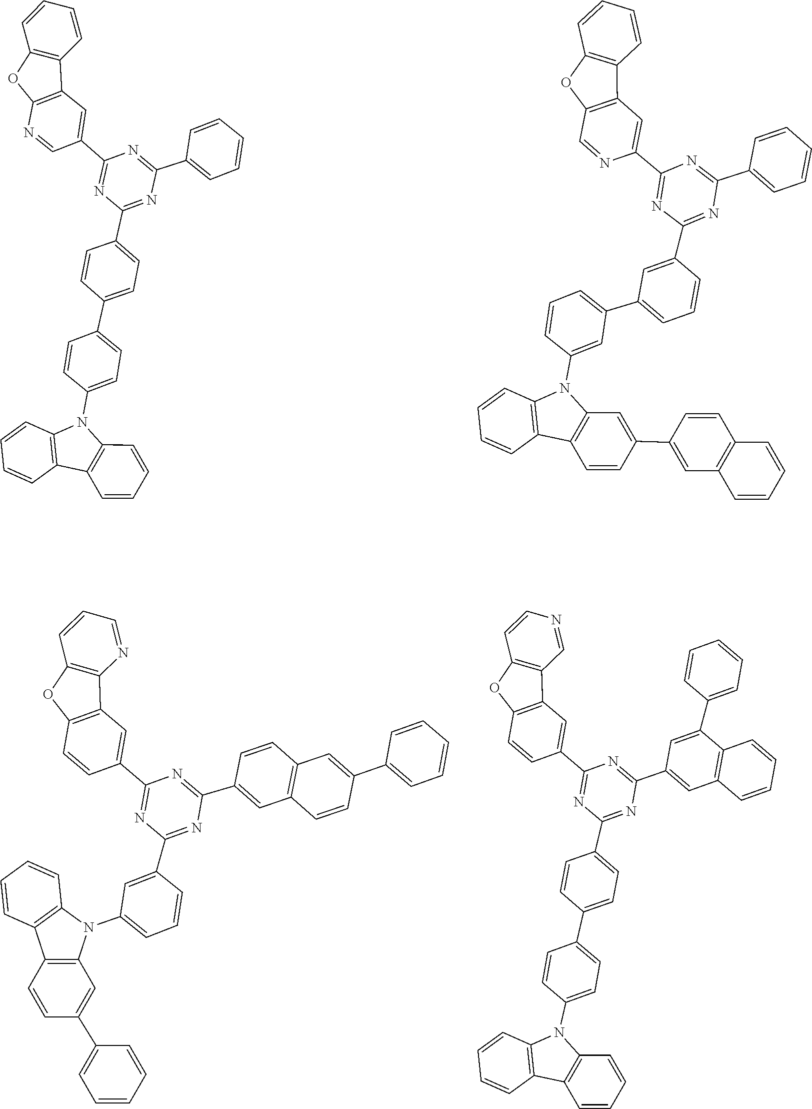 [SMILES: c1ccc(-c2nc(-c3ccc(-c4ccc(-n5c6ccccc6c6ccccc65)cc4)cc3)nc(-c3cnc4oc5ccccc5c4c3)n2)cc1 ; c1ccc(-c2nc(-c3cccc(-c4cccc(-n5c6ccccc6c6ccc(-c7ccc8ccccc8c7)cc65)c4)c3)nc(-c3cc4c(cn3)oc3ccccc34)n2)cc1 ; c1ccc(-c2ccc3cc(-c4nc(-c5cccc(-n6c7ccccc7c7ccc(-c8ccccc8)cc76)c5)nc(-c5ccc6oc7cccnc7c6c5)n4)ccc3c2)cc1 ; c1ccc(-c2cc(-c3nc(-c4ccc(-c5ccc(-n6c7ccccc7c7ccccc76)cc5)cc4)nc(-c4ccc5oc6ccncc6c5c4)n3)cc3ccccc23)cc1]

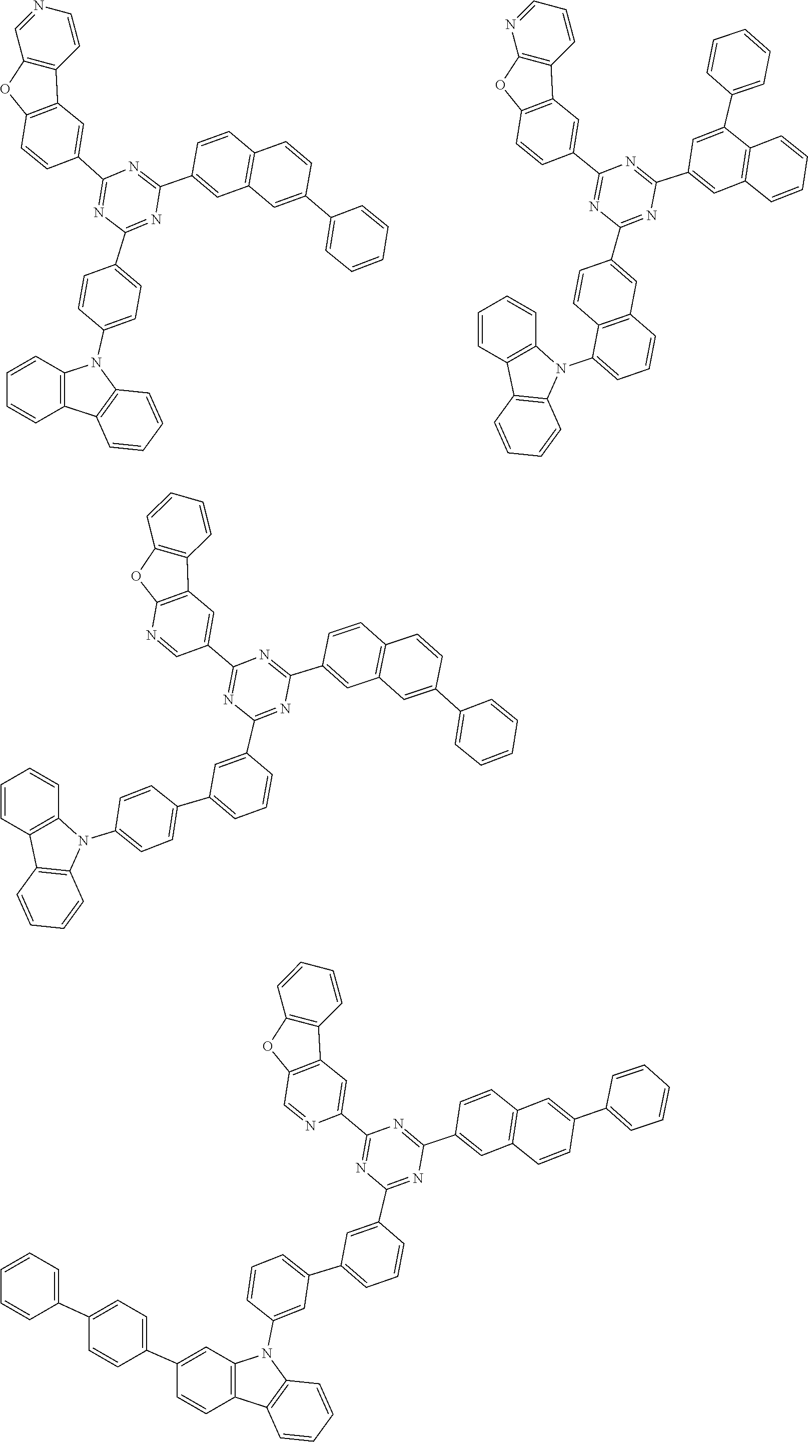 [SMILES: c1ccc(-c2ccc3ccc(-c4nc(-c5ccc(-n6c7ccccc7c7ccccc76)cc5)nc(-c5ccc6oc7cnccc7c6c5)n4)cc3c2)cc1 ; c1ccc(-c2cc(-c3nc(-c4ccc5c(-n6c7ccccc7c7ccccc76)cccc5c4)nc(-c4ccc5oc6ncccc6c5c4)n3)cc3ccccc23)cc1 ; c1ccc(-c2ccc3ccc(-c4nc(-c5cccc(-c6ccc(-n7c8ccccc8c8ccccc87)cc6)c5)nc(-c5cnc6oc7ccccc7c6c5)n4)cc3c2)cc1 ; c1ccc(-c2ccc(-c3ccc4c5ccccc5n(-c5cccc(-c6cccc(-c7nc(-c8ccc9cc(-c%10ccccc%10)ccc9c8)nc(-c8cc9c(cn8)oc8ccccc89)n7)c6)c5)c4c3)cc2)cc1]

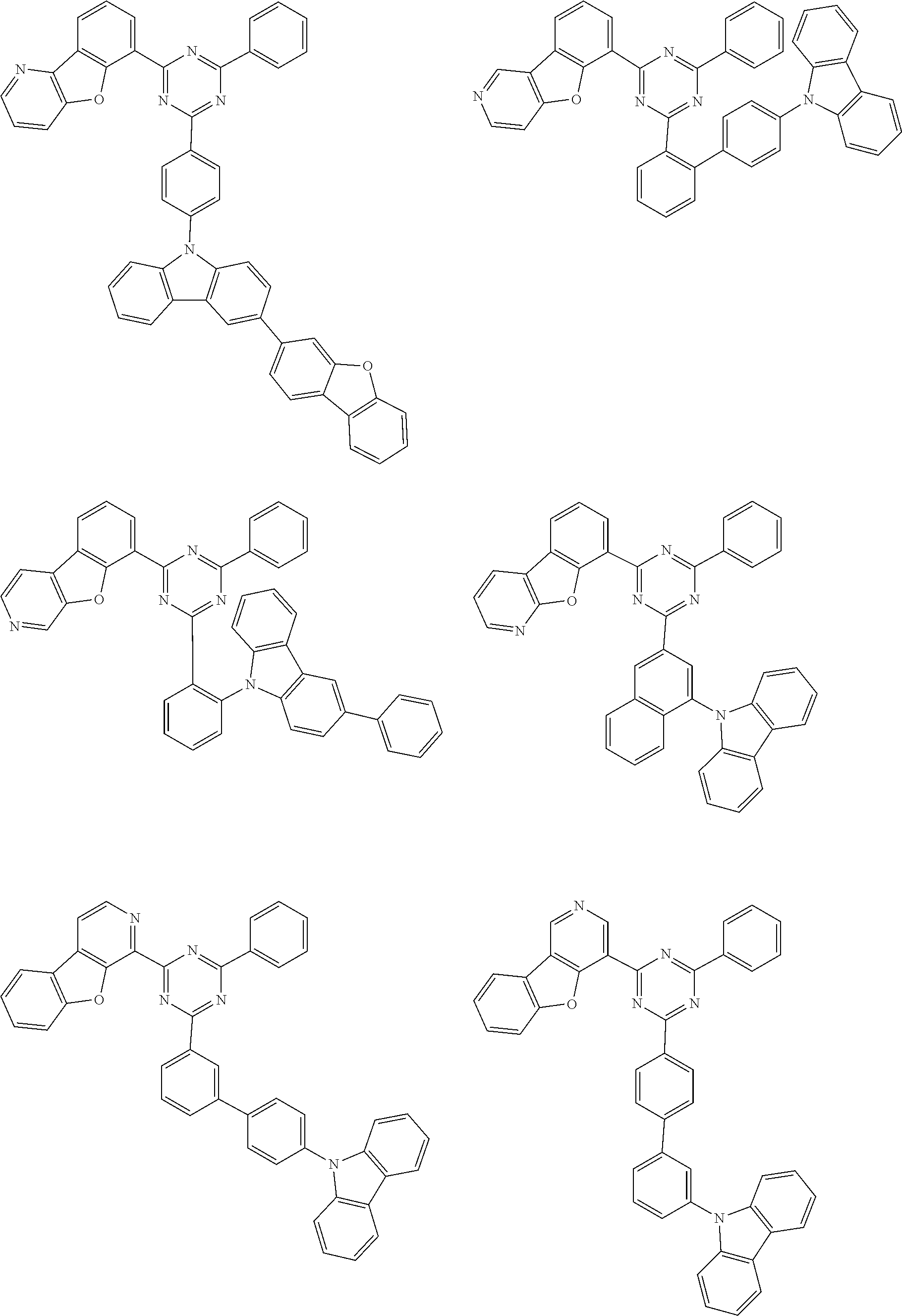

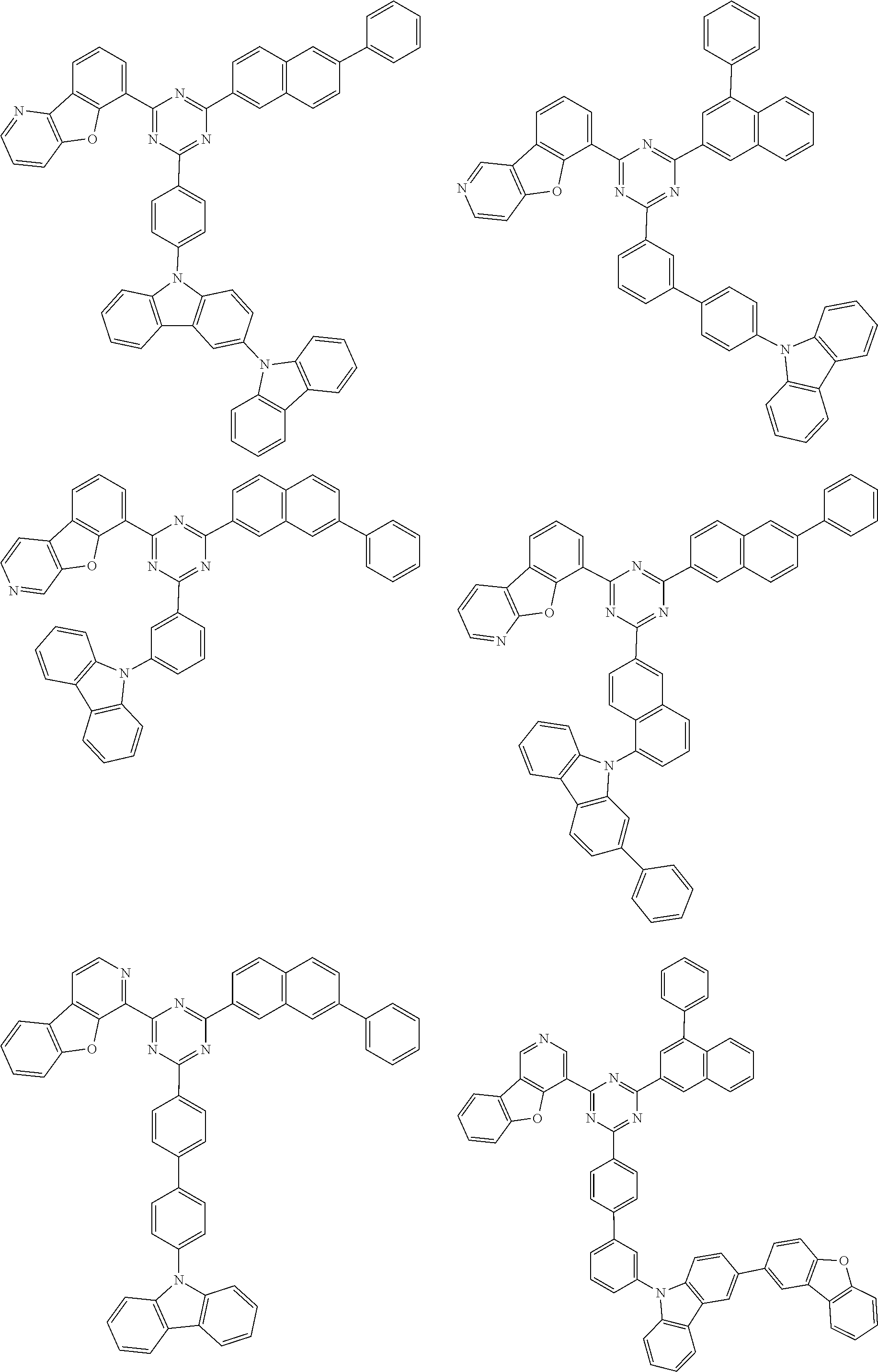

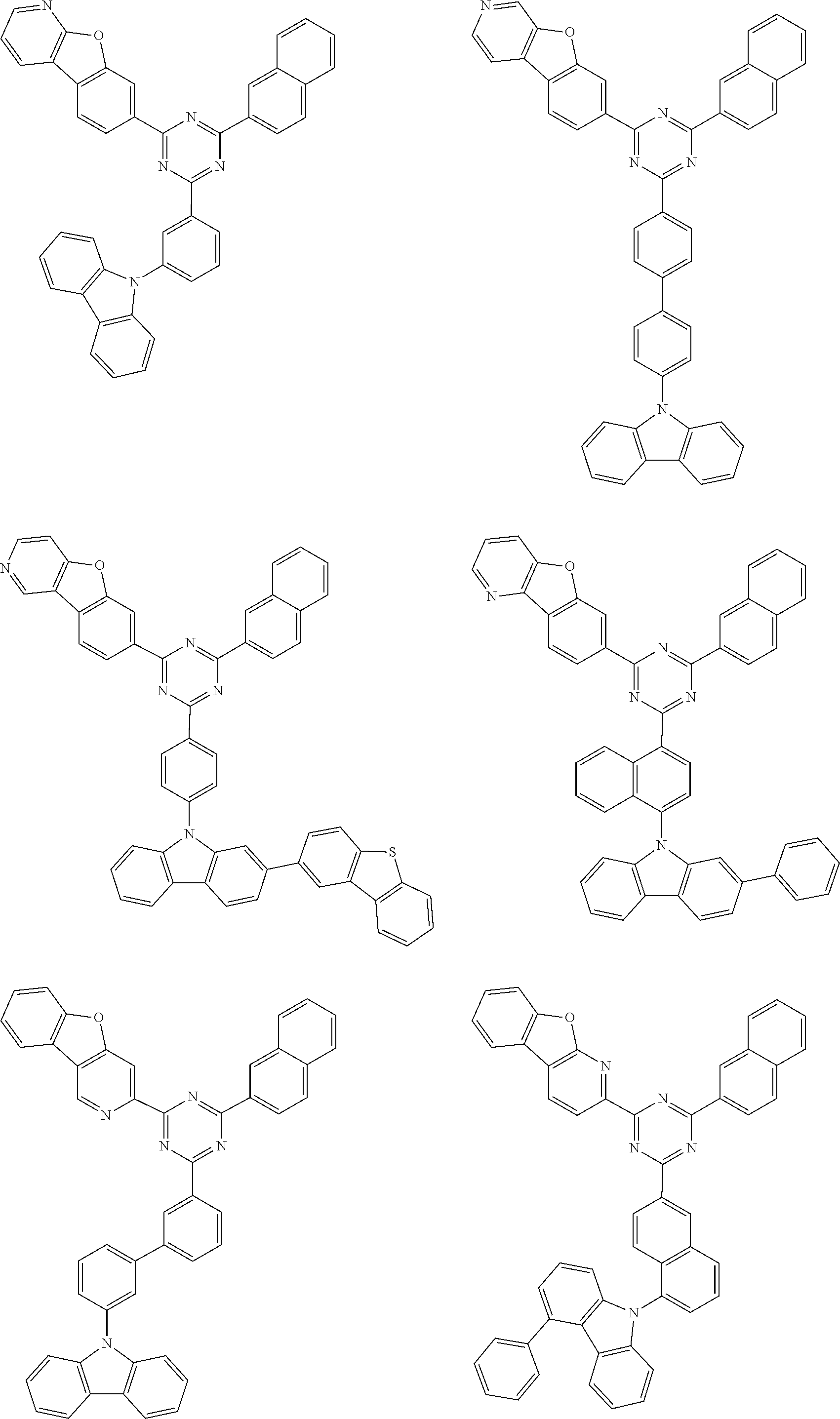

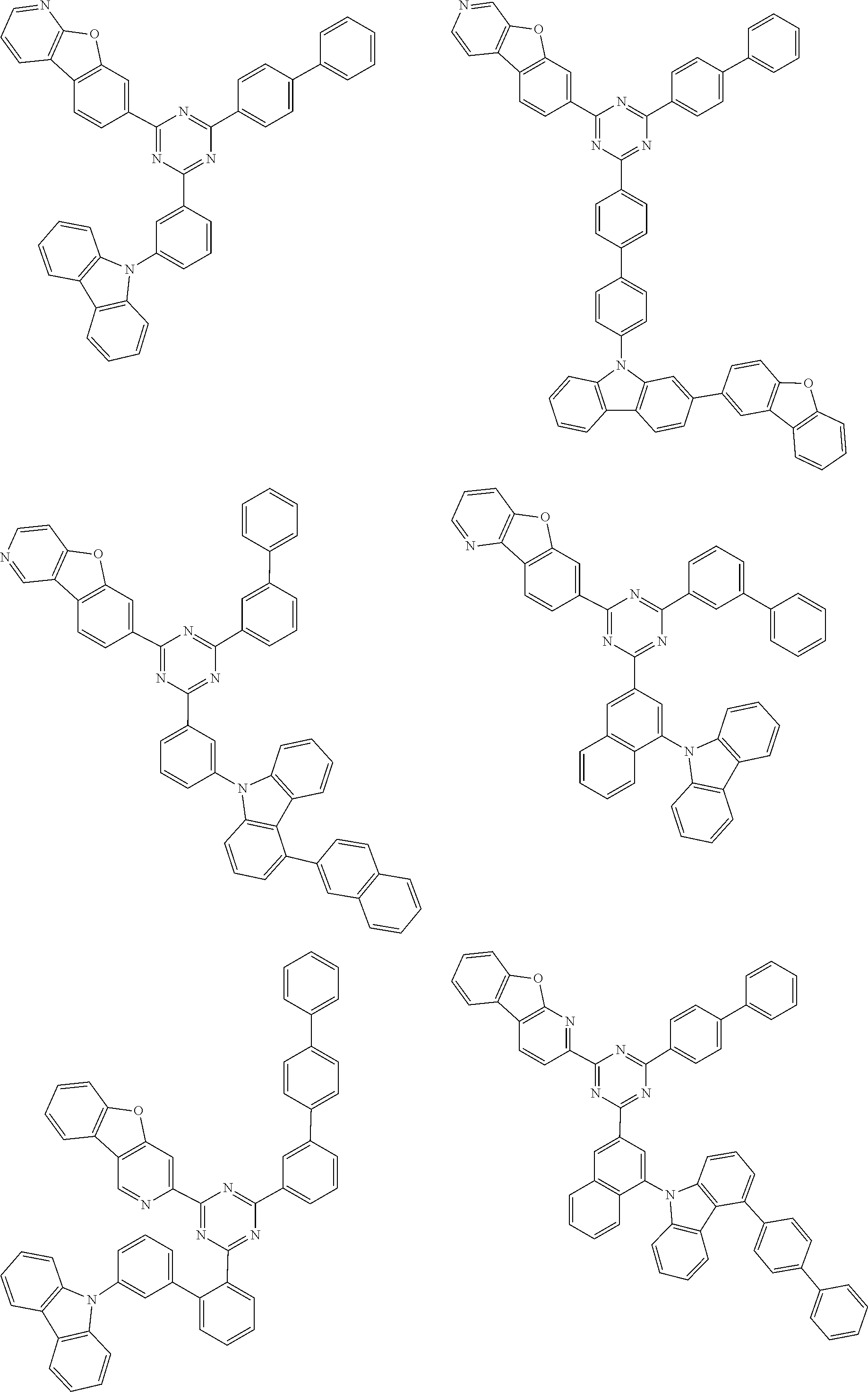

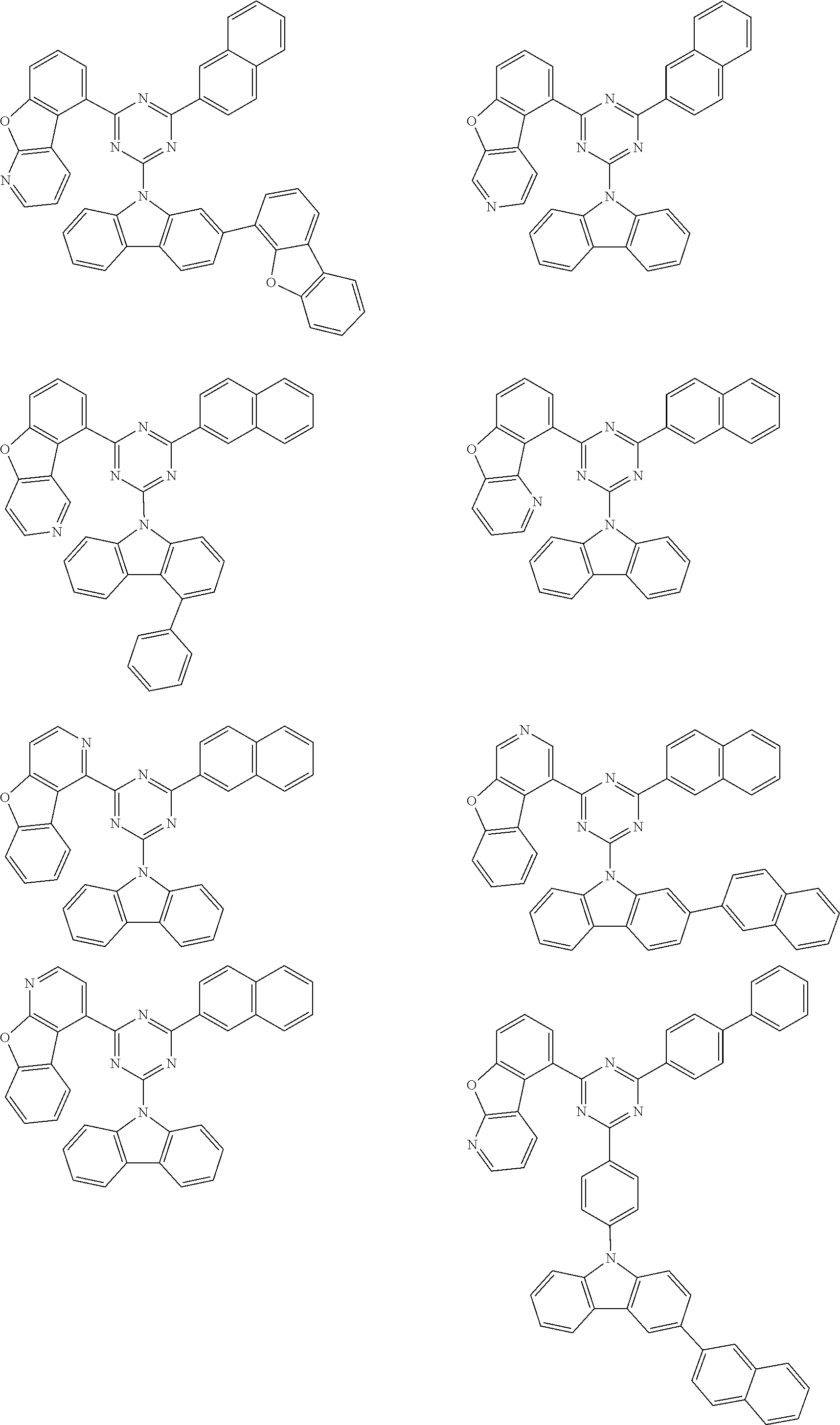

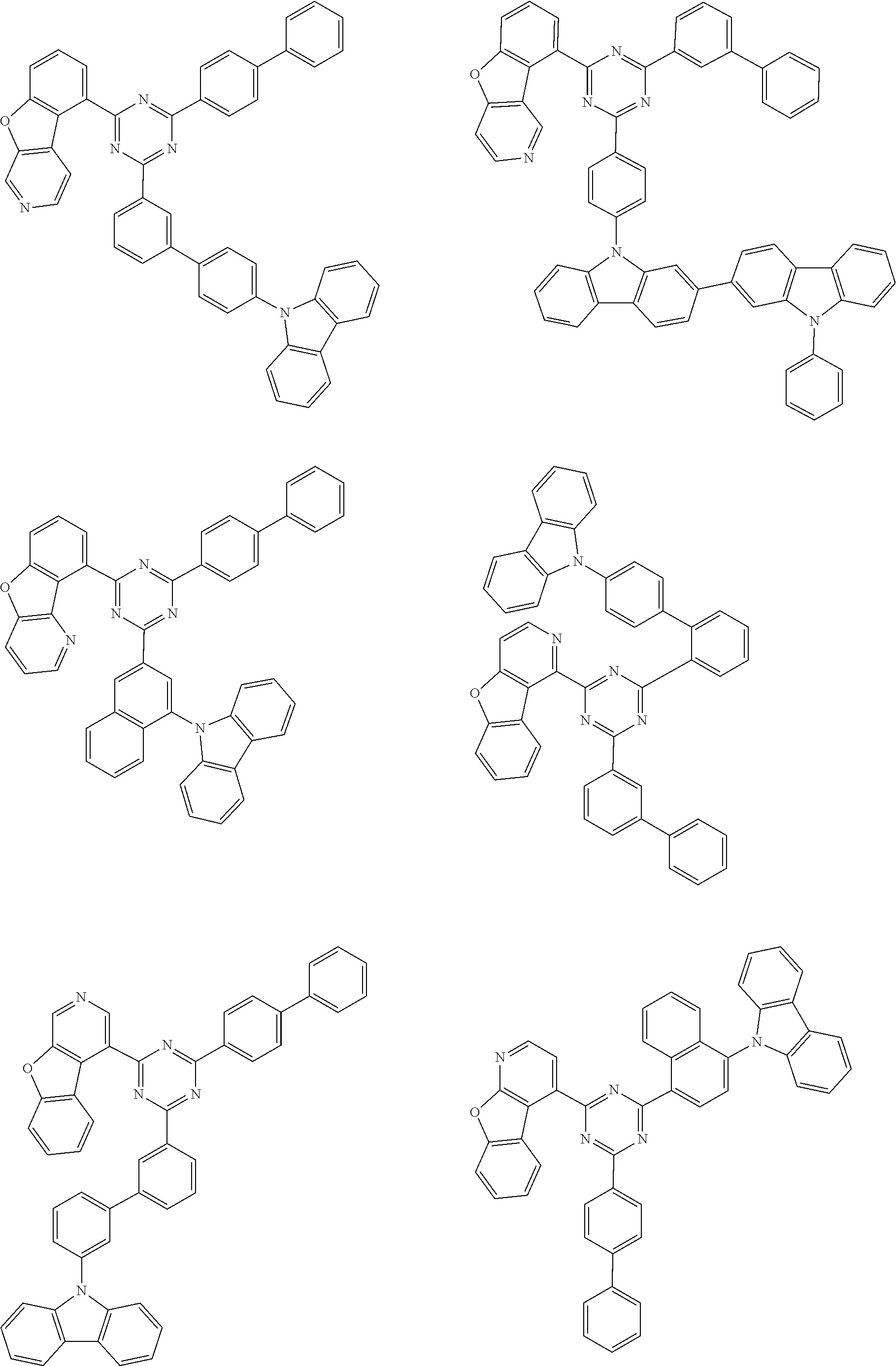

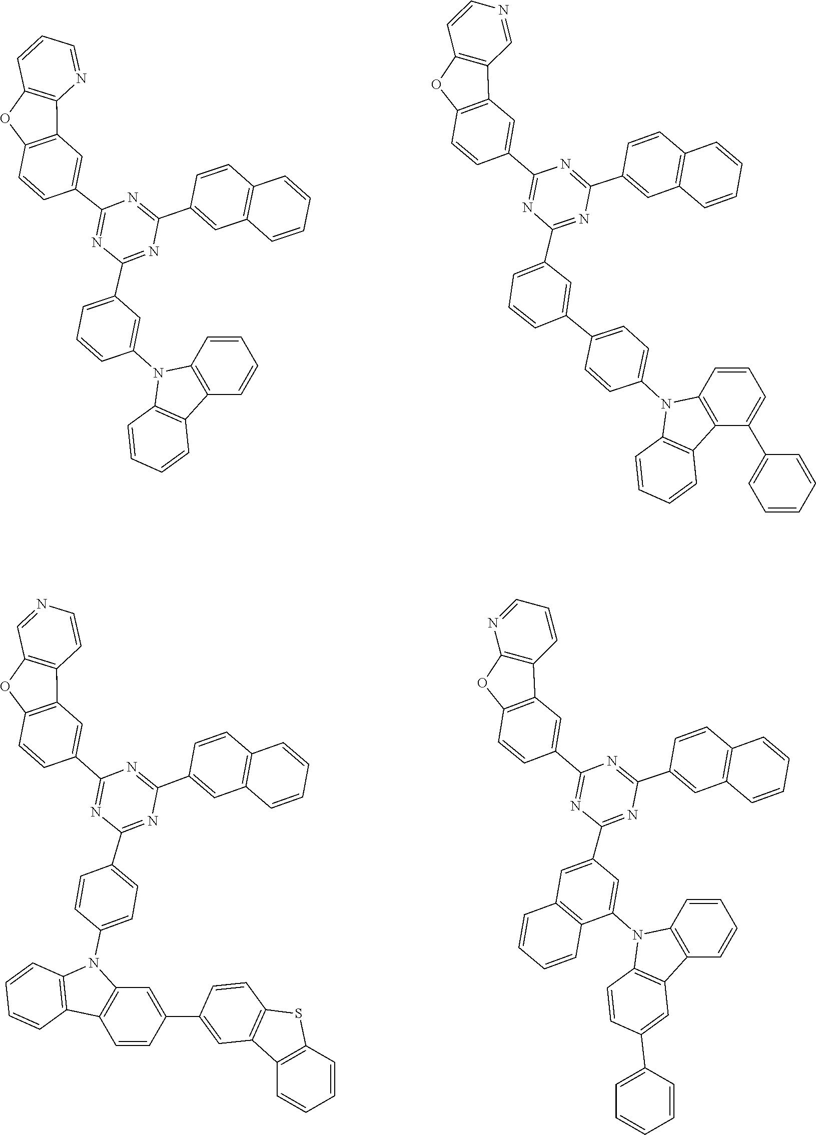 [SMILES: c1cc(-c2nc(-c3ccc4ccccc4c3)nc(-c3ccc4oc5cccnc5c4c3)n2)cc(-n2c3ccccc3c3ccccc32)c1 ; c1ccc(-c2cccc3c2c2ccccc2n3-c2ccc(-c3cccc(-c4nc(-c5ccc6ccccc6c5)nc(-c5ccc6oc7ccncc7c6c5)n4)c3)cc2)cc1 ; c1ccc2cc(-c3nc(-c4ccc(-n5c6ccccc6c6ccc(-c7ccc8sc9ccccc9c8c7)cc65)cc4)nc(-c4ccc5oc6cnccc6c5c4)n3)ccc2c1 ; c1ccc(-c2ccc3c(c2)c2ccccc2n3-c2cc(-c3nc(-c4ccc5ccccc5c4)nc(-c4ccc5oc6ncccc6c5c4)n3)cc3ccccc23)cc1]

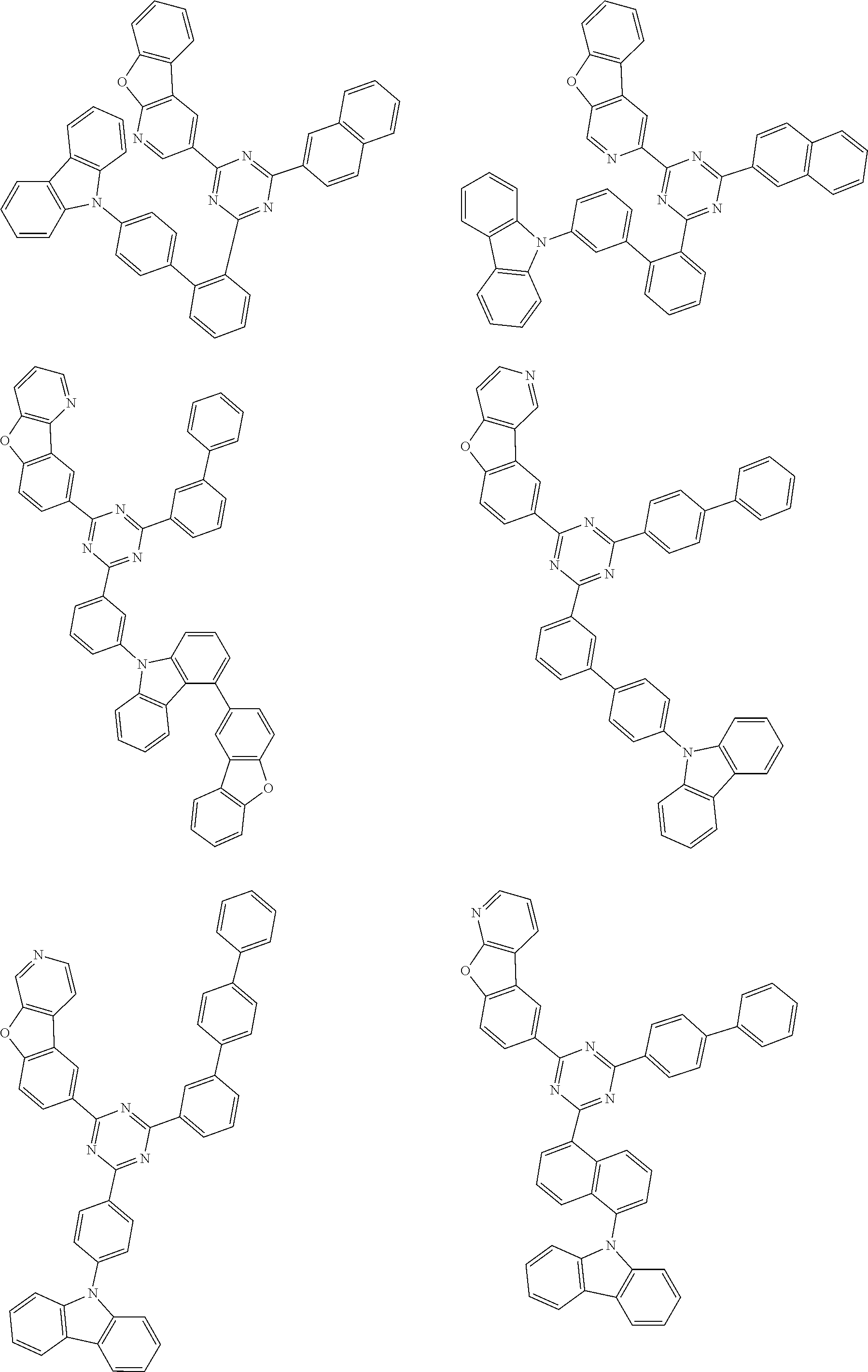

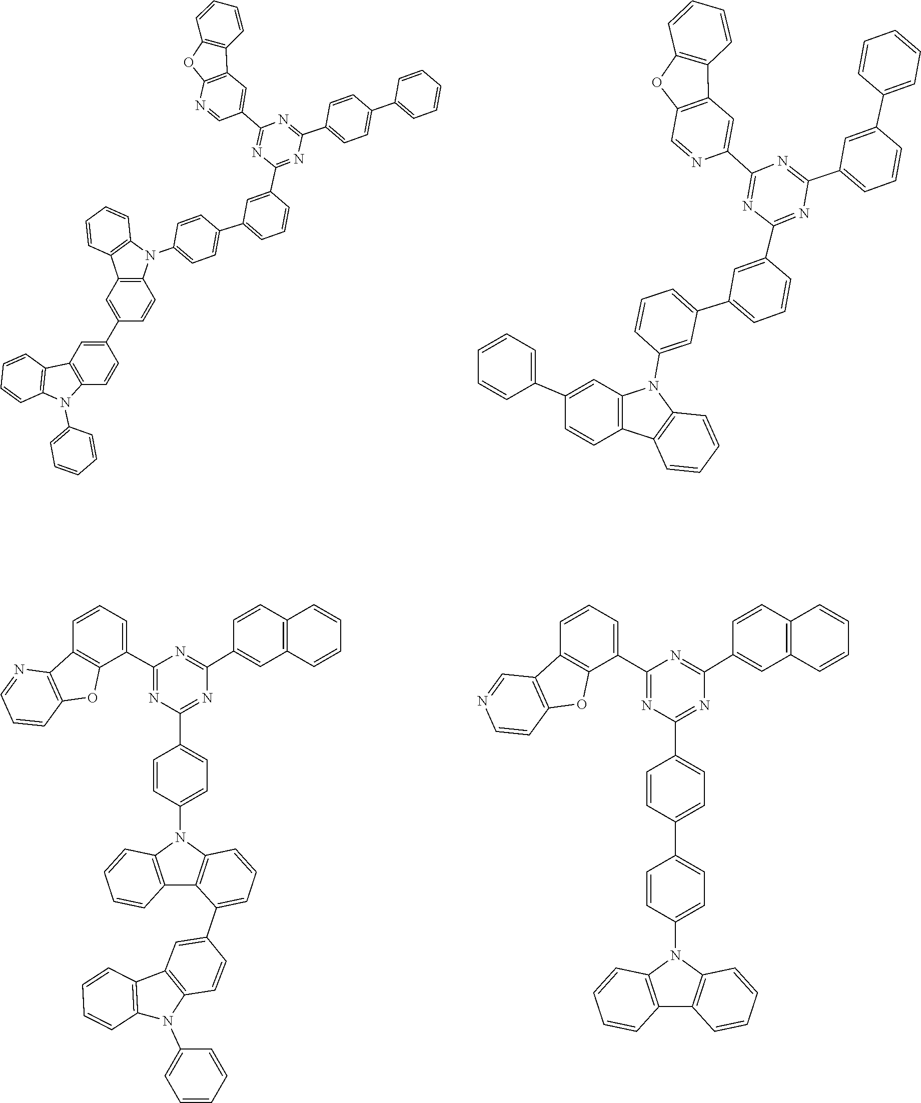 [SMILES: c1ccc(-c2ccc(-c3nc(-c4cccc(-c5ccc(-n6c7ccccc7c7cc(-c8ccc9c(c8)c8ccccc8n9-c8ccccc8)ccc76)cc5)c4)nc(-c4cnc5oc6ccccc6c5c4)n3)cc2)cc1 ; c1ccc(-c2cccc(-c3nc(-c4cccc(-c5cccc(-n6c7ccccc7c7ccc(-c8ccccc8)cc76)c5)c4)nc(-c4cc5c(cn4)oc4ccccc45)n3)c2)cc1 ; c1ccc(-n2c3ccccc3c3cc(-c4cccc5c4c4ccccc4n5-c4ccc(-c5nc(-c6ccc7ccccc7c6)nc(-c6cccc7c6oc6cccnc67)n5)cc4)ccc32)cc1 ; c1ccc2cc(-c3nc(-c4ccc(-c5ccc(-n6c7ccccc7c7ccccc76)cc5)cc4)nc(-c4cccc5c4oc4ccncc45)n3)ccc2c1]

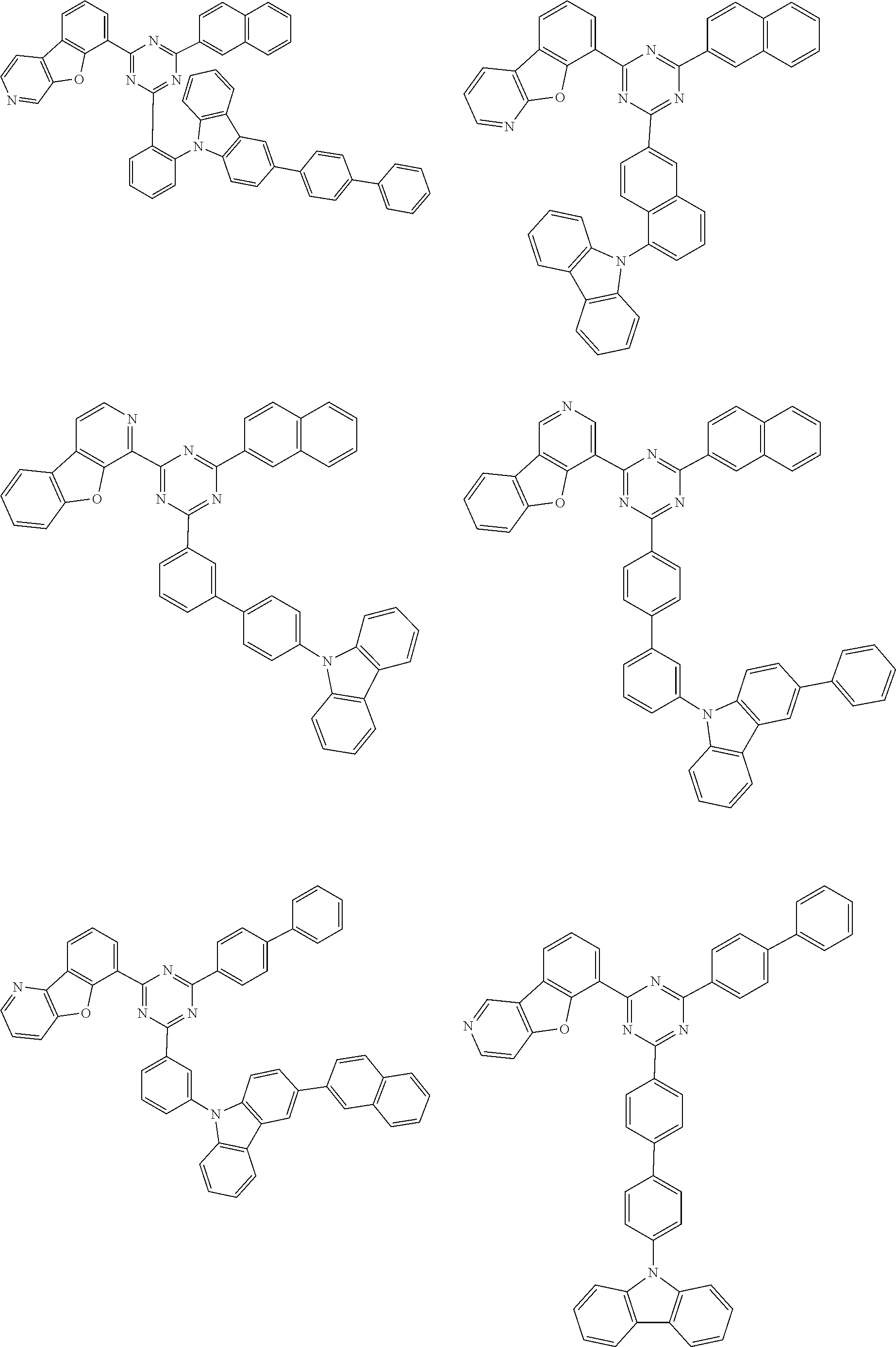

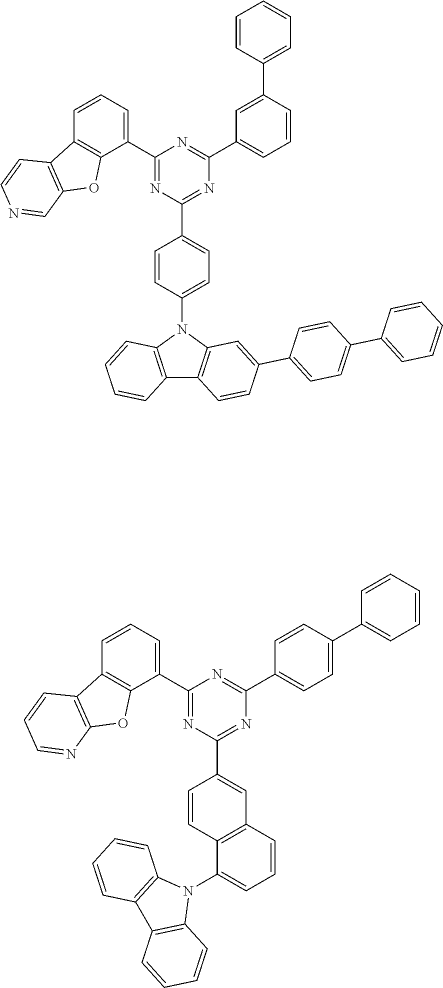 [SMILES: c1ccc(-c2ccc(-c3ccc4c5ccccc5n(-c5ccc(-c6nc(-c7cccc(-c8ccccc8)c7)nc(-c7cccc8c7oc7cnccc78)n6)cc5)c4c3)cc2)cc1 ; c1ccc(-c2ccc(-c3nc(-c4ccc5c(-n6c7ccccc7c7ccccc76)cccc5c4)nc(-c4cccc5c4oc4ncccc45)n3)cc2)cc1]

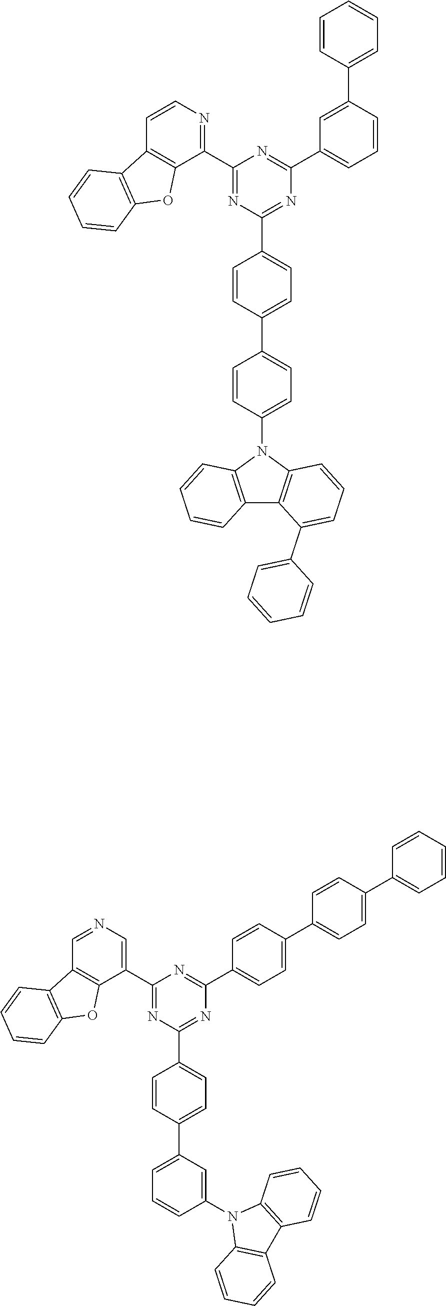 [SMILES: c1ccc(-c2cccc(-c3nc(-c4ccc(-c5ccc(-n6c7ccccc7c7c(-c8ccccc8)cccc76)cc5)cc4)nc(-c4nccc5c4oc4ccccc45)n3)c2)cc1 ; c1ccc(-c2ccc(-c3ccc(-c4nc(-c5ccc(-c6cccc(-n7c8ccccc8c8ccccc87)c6)cc5)nc(-c5cncc6c5oc5ccccc56)n4)cc3)cc2)cc1]

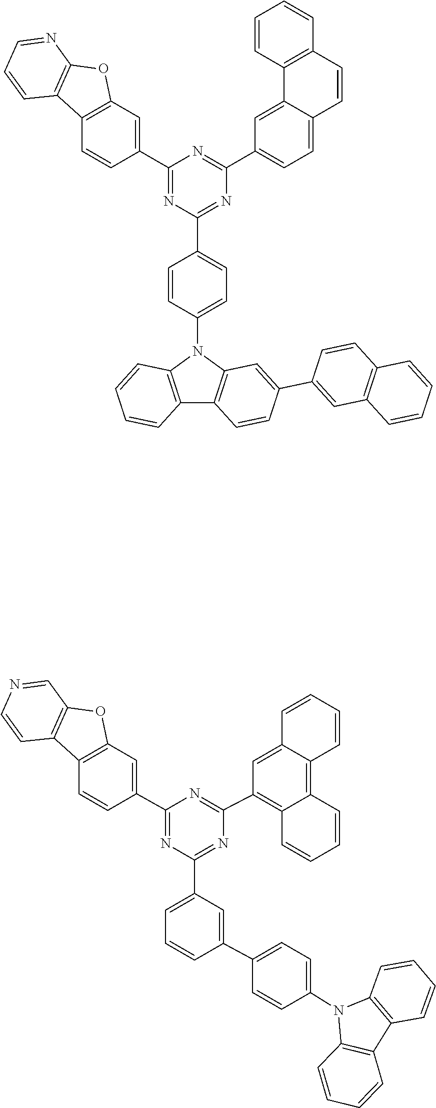 [SMILES: c1ccc2cc(-c3ccc4c5ccccc5n(-c5ccc(-c6nc(-c7ccc8c(c7)oc7ncccc78)nc(-c7ccc8ccc9ccccc9c8c7)n6)cc5)c4c3)ccc2c1 ; c1cc(-c2ccc(-n3c4ccccc4c4ccccc43)cc2)cc(-c2nc(-c3ccc4c(c3)oc3cnccc34)nc(-c3cc4ccccc4c4ccccc34)n2)c1]

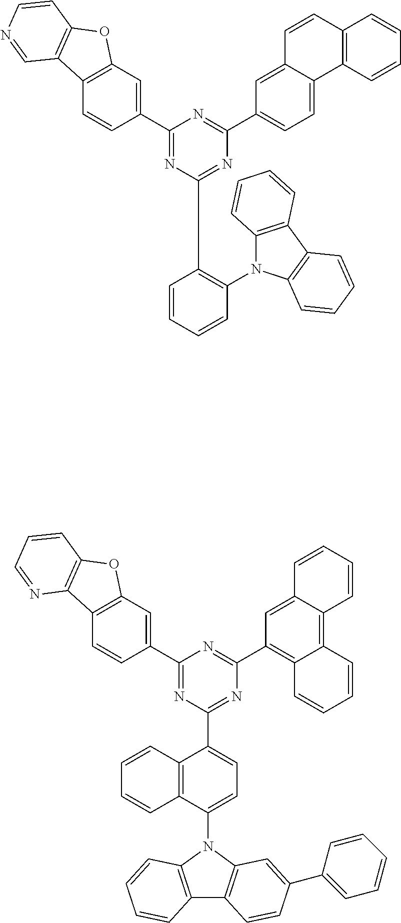 [SMILES: c1ccc(-n2c3ccccc3c3ccccc32)c(-c2nc(-c3ccc4c(ccc5ccccc54)c3)nc(-c3ccc4c(c3)oc3ccncc34)n2)c1 ; c1ccc(-c2ccc3c4ccccc4n(-c4ccc(-c5nc(-c6ccc7c(c6)oc6cccnc67)nc(-c6cc7ccccc7c7ccccc67)n5)c5ccccc45)c3c2)cc1]

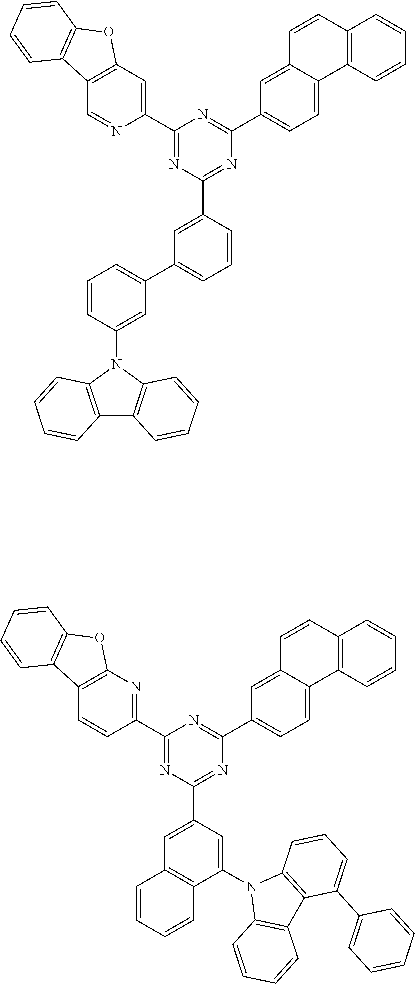 [SMILES: c1cc(-c2cccc(-n3c4ccccc4c4ccccc43)c2)cc(-c2nc(-c3ccc4c(ccc5ccccc54)c3)nc(-c3cc4oc5ccccc5c4cn3)n2)c1 ; c1ccc(-c2cccc3c2c2ccccc2n3-c2cc(-c3nc(-c4ccc5c(ccc6ccccc65)c4)nc(-c4ccc5c(n4)oc4ccccc45)n3)cc3ccccc23)cc1]

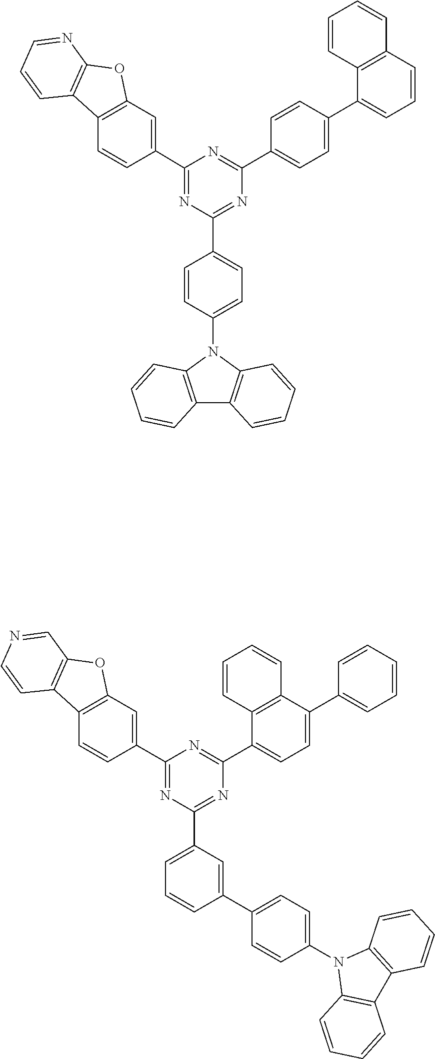 [SMILES: c1ccc2c(-c3ccc(-c4nc(-c5ccc(-n6c7ccccc7c7ccccc76)cc5)nc(-c5ccc6c(c5)oc5ncccc56)n4)cc3)cccc2c1 ; c1ccc(-c2ccc(-c3nc(-c4cccc(-c5ccc(-n6c7ccccc7c7ccccc76)cc5)c4)nc(-c4ccc5c(c4)oc4cnccc45)n3)c3ccccc23)cc1]

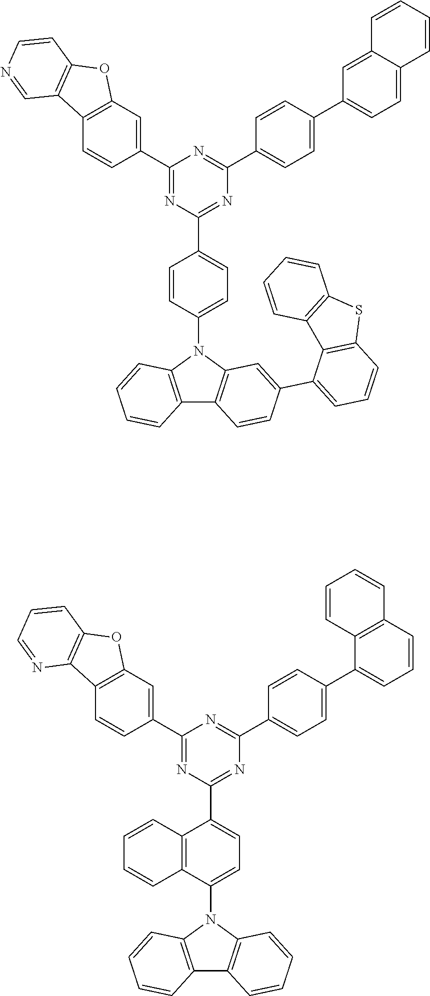 [SMILES: c1ccc2cc(-c3ccc(-c4nc(-c5ccc(-n6c7ccccc7c7ccc(-c8cccc9sc%10ccccc%10c89)cc76)cc5)nc(-c5ccc6c(c5)oc5ccncc56)n4)cc3)ccc2c1 ; c1ccc2c(-c3ccc(-c4nc(-c5ccc6c(c5)oc5cccnc56)nc(-c5ccc(-n6c7ccccc7c7ccccc76)c6ccccc56)n4)cc3)cccc2c1]

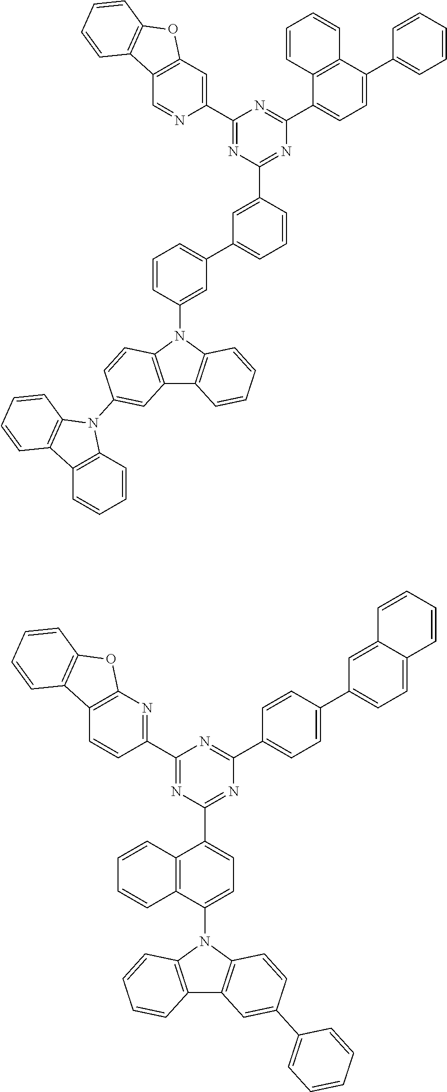 [SMILES: c1ccc(-c2ccc(-c3nc(-c4cccc(-c5cccc(-n6c7ccccc7c7cc(-n8c9ccccc9c9ccccc98)ccc76)c5)c4)nc(-c4cc5oc6ccccc6c5cn4)n3)c3ccccc23)cc1 ; c1ccc(-c2ccc3c(c2)c2ccccc2n3-c2ccc(-c3nc(-c4ccc(-c5ccc6ccccc6c5)cc4)nc(-c4ccc5c(n4)oc4ccccc45)n3)c3ccccc23)cc1]

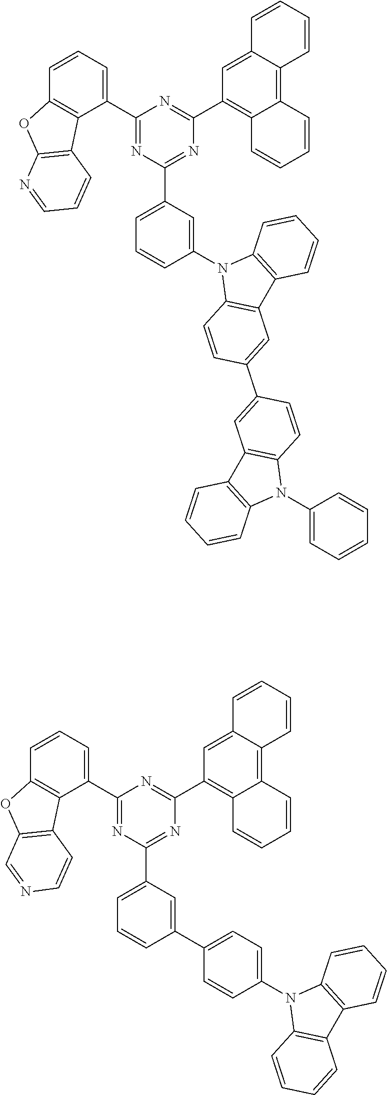 [SMILES: c1ccc(-n2c3ccccc3c3cc(-c4ccc5c(c4)c4ccccc4n5-c4cccc(-c5nc(-c6cc7ccccc7c7ccccc67)nc(-c6cccc7oc8ncccc8c67)n5)c4)ccc32)cc1 ; c1cc(-c2ccc(-n3c4ccccc4c4ccccc43)cc2)cc(-c2nc(-c3cc4ccccc4c4ccccc34)nc(-c3cccc4oc5cnccc5c34)n2)c1]

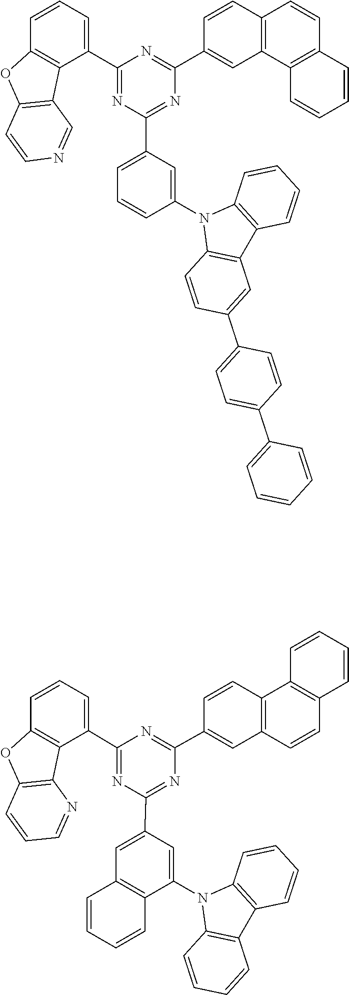 [SMILES: c1ccc(-c2ccc(-c3ccc4c(c3)c3ccccc3n4-c3cccc(-c4nc(-c5ccc6ccc7ccccc7c6c5)nc(-c5cccc6oc7ccncc7c56)n4)c3)cc2)cc1 ; c1ccc2c(-n3c4ccccc4c4ccccc43)cc(-c3nc(-c4ccc5c(ccc6ccccc65)c4)nc(-c4cccc5oc6cccnc6c45)n3)cc2c1]

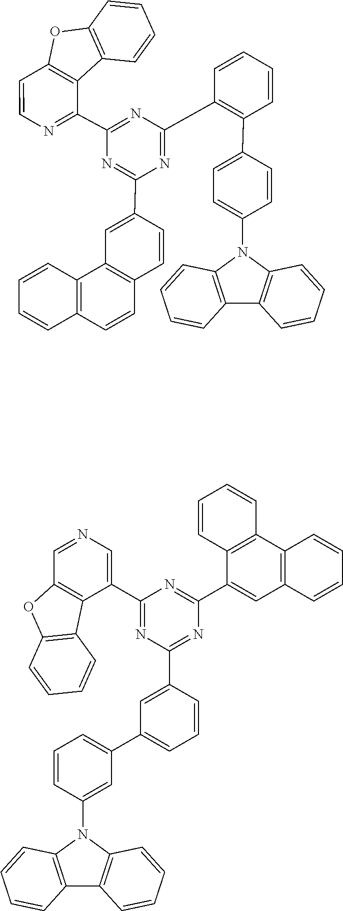 [SMILES: c1ccc(-c2nc(-c3ccc4ccc5ccccc5c4c3)nc(-c3nccc4oc5ccccc5c34)n2)c(-c2ccc(-n3c4ccccc4c4ccccc43)cc2)c1 ; c1cc(-c2cccc(-n3c4ccccc4c4ccccc43)c2)cc(-c2nc(-c3cc4ccccc4c4ccccc34)nc(-c3cncc4oc5ccccc5c34)n2)c1]

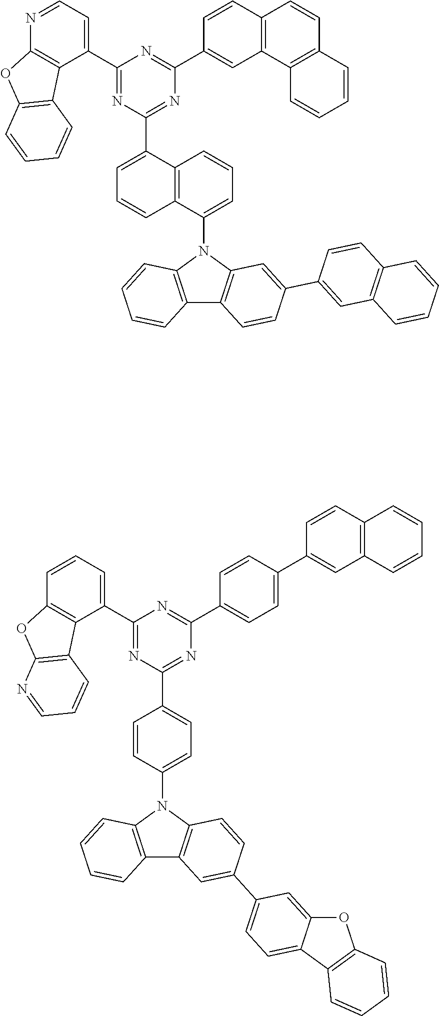 [SMILES: c1ccc2cc(-c3ccc4c5ccccc5n(-c5cccc6c(-c7nc(-c8ccc9ccc%10ccccc%10c9c8)nc(-c8ccnc9oc%10ccccc%10c89)n7)cccc56)c4c3)ccc2c1 ; c1ccc2cc(-c3ccc(-c4nc(-c5ccc(-n6c7ccccc7c7cc(-c8ccc9c(c8)oc8ccccc89)ccc76)cc5)nc(-c5cccc6oc7ncccc7c56)n4)cc3)ccc2c1]

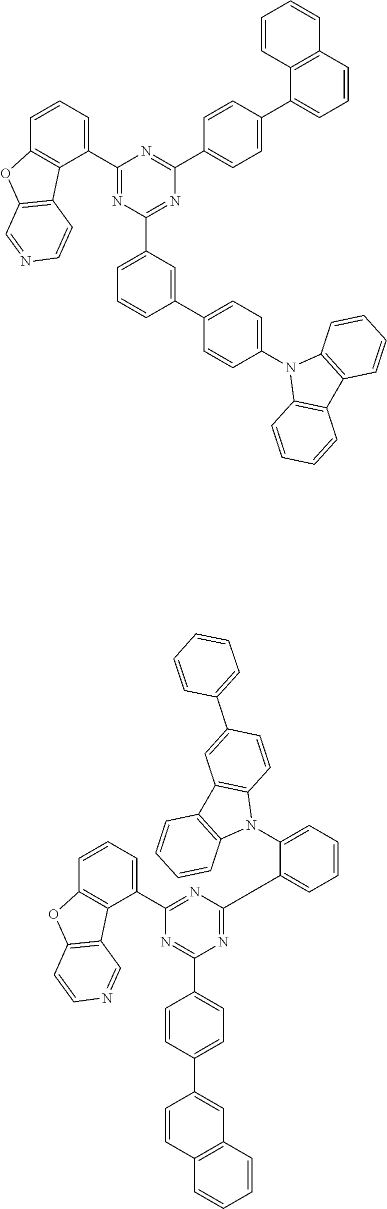 [SMILES: c1cc(-c2ccc(-n3c4ccccc4c4ccccc43)cc2)cc(-c2nc(-c3ccc(-c4cccc5ccccc45)cc3)nc(-c3cccc4oc5cnccc5c34)n2)c1 ; c1ccc(-c2ccc3c(c2)c2ccccc2n3-c2ccccc2-c2nc(-c3ccc(-c4ccc5ccccc5c4)cc3)nc(-c3cccc4oc5ccncc5c34)n2)cc1]

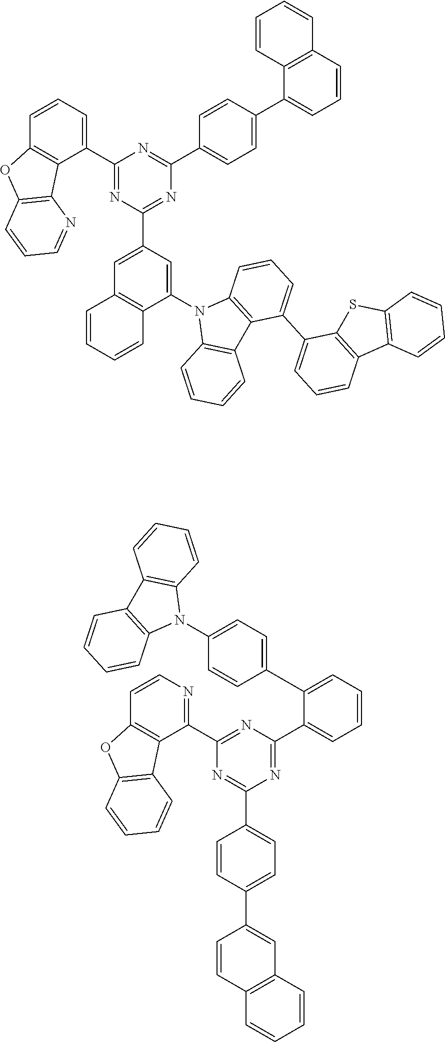 [SMILES: c1ccc2c(-c3ccc(-c4nc(-c5cc(-n6c7ccccc7c7c(-c8cccc9c8sc8ccccc89)cccc76)c6ccccc6c5)nc(-c5cccc6oc7cccnc7c56)n4)cc3)cccc2c1 ; c1ccc(-c2nc(-c3ccc(-c4ccc5ccccc5c4)cc3)nc(-c3nccc4oc5ccccc5c34)n2)c(-c2ccc(-n3c4ccccc4c4ccccc43)cc2)c1]

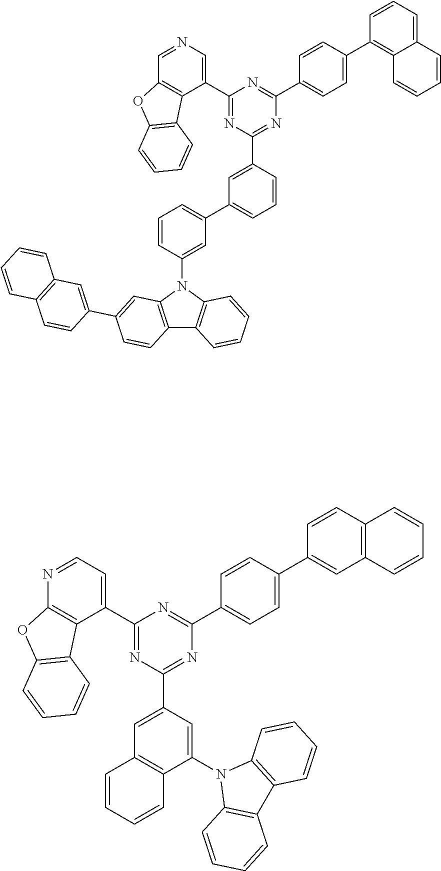 [SMILES: c1cc(-c2cccc(-n3c4ccccc4c4ccc(-c5ccc6ccccc6c5)cc43)c2)cc(-c2nc(-c3ccc(-c4cccc5ccccc45)cc3)nc(-c3cncc4oc5ccccc5c34)n2)c1 ; c1ccc2cc(-c3ccc(-c4nc(-c5cc(-n6c7ccccc7c7ccccc76)c6ccccc6c5)nc(-c5ccnc6oc7ccccc7c56)n4)cc3)ccc2c1]

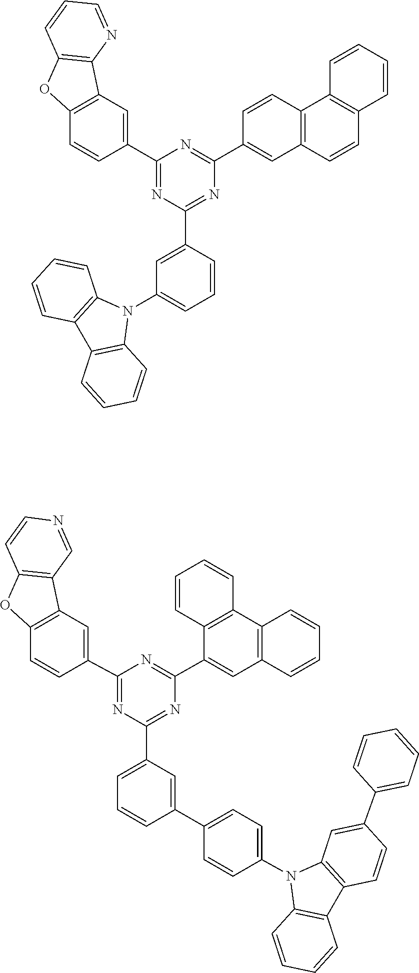 [SMILES: c1cc(-c2nc(-c3ccc4c(ccc5ccccc54)c3)nc(-c3ccc4oc5cccnc5c4c3)n2)cc(-n2c3ccccc3c3ccccc32)c1 ; c1ccc(-c2ccc3c4ccccc4n(-c4ccc(-c5cccc(-c6nc(-c7ccc8oc9ccncc9c8c7)nc(-c7cc8ccccc8c8ccccc78)n6)c5)cc4)c3c2)cc1]

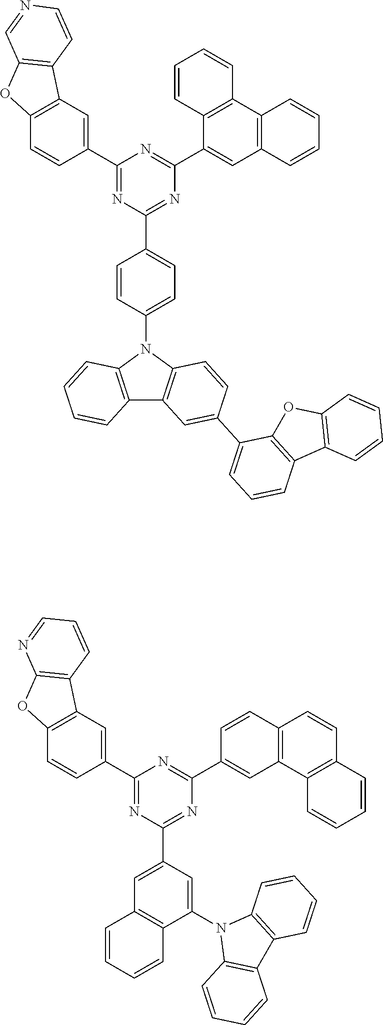 [SMILES: c1ccc2c(c1)cc(-c1nc(-c3ccc(-n4c5ccccc5c5cc(-c6cccc7c6oc6ccccc67)ccc54)cc3)nc(-c3ccc4oc5cnccc5c4c3)n1)c1ccccc12 ; c1ccc2c(-n3c4ccccc4c4ccccc43)cc(-c3nc(-c4ccc5ccc6ccccc6c5c4)nc(-c4ccc5oc6ncccc6c5c4)n3)cc2c1]

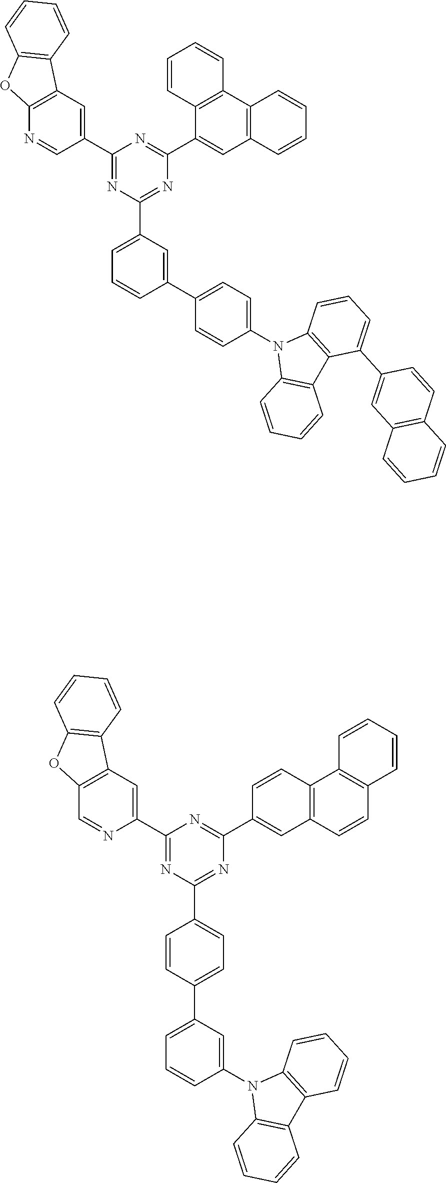 [SMILES: c1cc(-c2ccc(-n3c4ccccc4c4c(-c5ccc6ccccc6c5)cccc43)cc2)cc(-c2nc(-c3cnc4oc5ccccc5c4c3)nc(-c3cc4ccccc4c4ccccc34)n2)c1 ; c1cc(-c2ccc(-c3nc(-c4ccc5c(ccc6ccccc65)c4)nc(-c4cc5c(cn4)oc4ccccc45)n3)cc2)cc(-n2c3ccccc3c3ccccc32)c1]

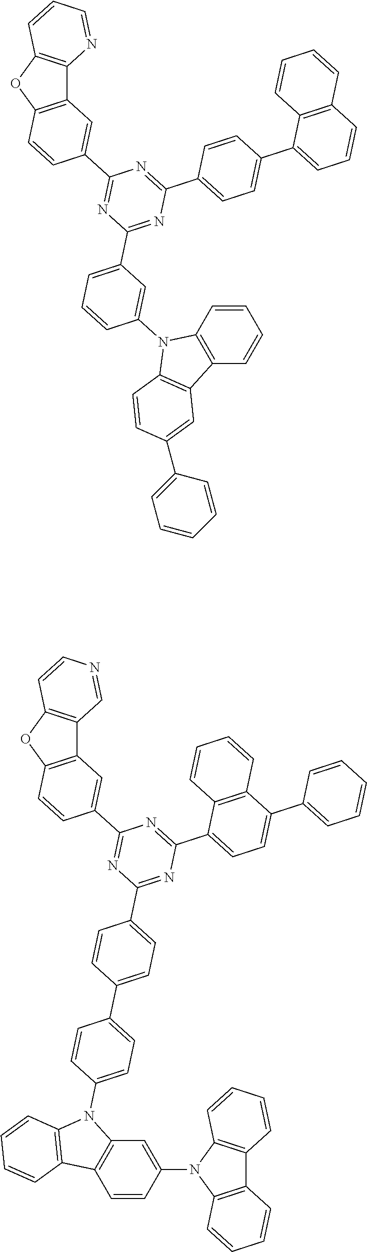 [SMILES: c1ccc(-c2ccc3c(c2)c2ccccc2n3-c2cccc(-c3nc(-c4ccc(-c5cccc6ccccc56)cc4)nc(-c4ccc5oc6cccnc6c5c4)n3)c2)cc1 ; c1ccc(-c2ccc(-c3nc(-c4ccc(-c5ccc(-n6c7ccccc7c7ccc(-n8c9ccccc9c9ccccc98)cc76)cc5)cc4)nc(-c4ccc5oc6ccncc6c5c4)n3)c3ccccc23)cc1]

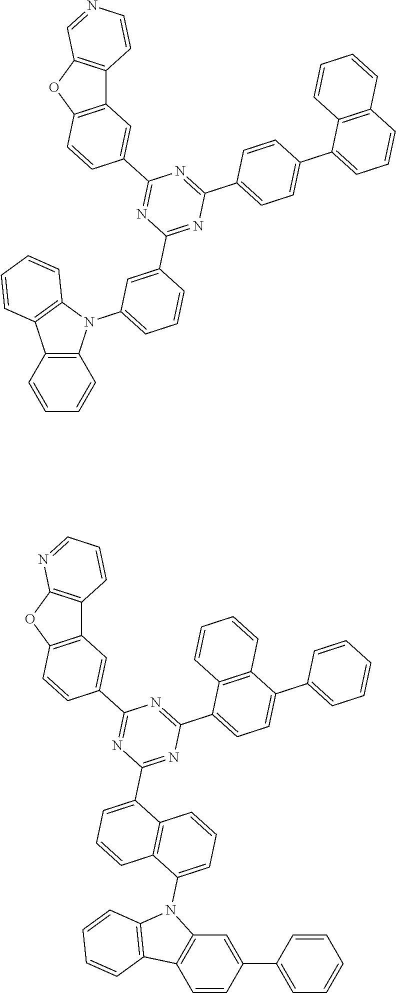 [SMILES: c1cc(-c2nc(-c3ccc(-c4cccc5ccccc45)cc3)nc(-c3ccc4oc5cnccc5c4c3)n2)cc(-n2c3ccccc3c3ccccc32)c1 ; c1ccc(-c2ccc3c4ccccc4n(-c4cccc5c(-c6nc(-c7ccc8oc9ncccc9c8c7)nc(-c7ccc(-c8ccccc8)c8ccccc78)n6)cccc45)c3c2)cc1]

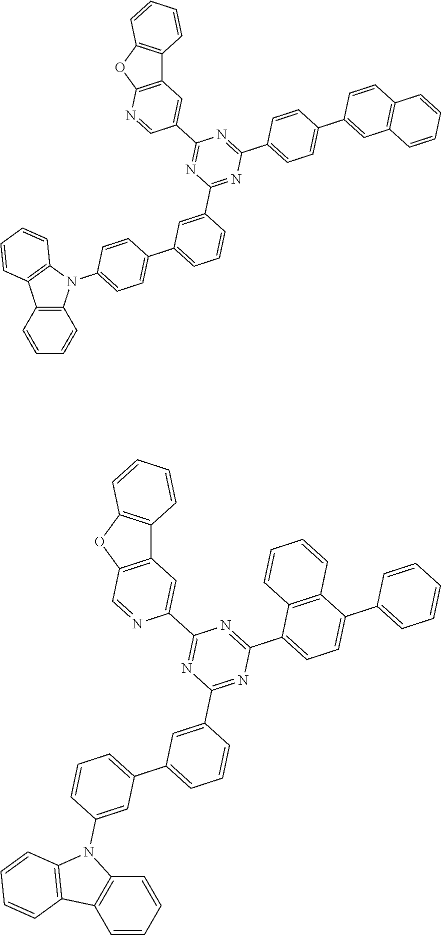 [SMILES: c1cc(-c2ccc(-n3c4ccccc4c4ccccc43)cc2)cc(-c2nc(-c3ccc(-c4ccc5ccccc5c4)cc3)nc(-c3cnc4oc5ccccc5c4c3)n2)c1 ; c1ccc(-c2ccc(-c3nc(-c4cccc(-c5cccc(-n6c7ccccc7c7ccccc76)c5)c4)nc(-c4cc5c(cn4)oc4ccccc45)n3)c3ccccc23)cc1]

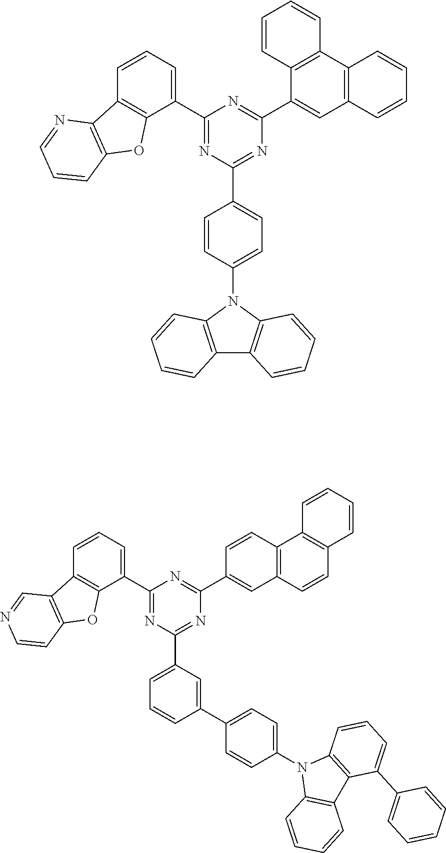 [SMILES: c1ccc2c(c1)cc(-c1nc(-c3ccc(-n4c5ccccc5c5ccccc54)cc3)nc(-c3cccc4c3oc3cccnc34)n1)c1ccccc12 ; c1ccc(-c2cccc3c2c2ccccc2n3-c2ccc(-c3cccc(-c4nc(-c5ccc6c(ccc7ccccc76)c5)nc(-c5cccc6c5oc5ccncc56)n4)c3)cc2)cc1]

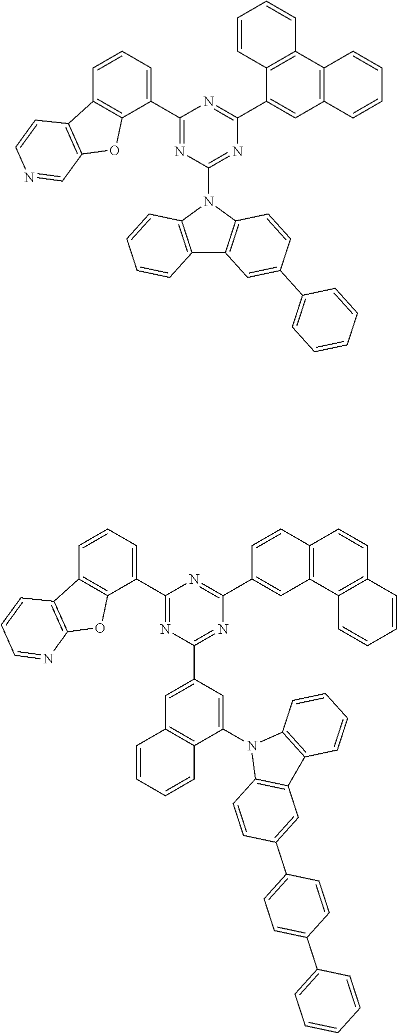 [SMILES: c1ccc(-c2ccc3c(c2)c2ccccc2n3-c2nc(-c3cc4ccccc4c4ccccc34)nc(-c3cccc4c3oc3cnccc34)n2)cc1 ; c1ccc(-c2ccc(-c3ccc4c(c3)c3ccccc3n4-c3cc(-c4nc(-c5ccc6ccc7ccccc7c6c5)nc(-c5cccc6c5oc5ncccc56)n4)cc4ccccc34)cc2)cc1]

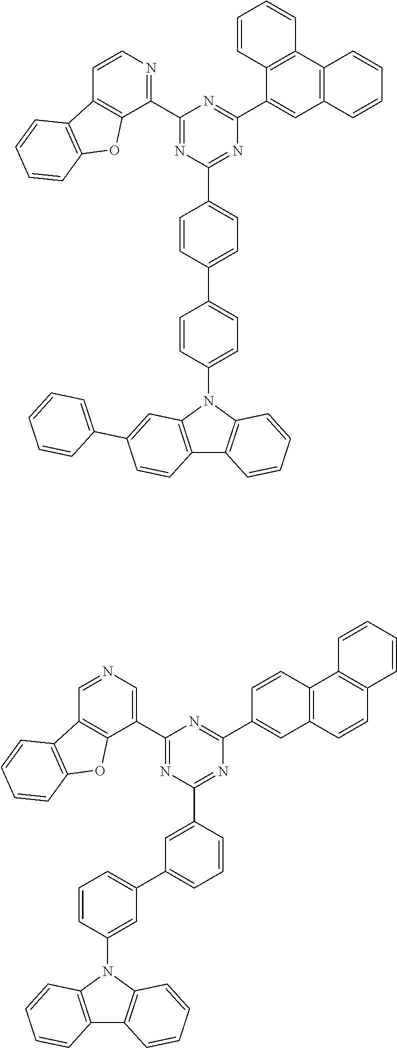 [SMILES: c1ccc(-c2ccc3c4ccccc4n(-c4ccc(-c5ccc(-c6nc(-c7cc8ccccc8c8ccccc78)nc(-c7nccc8c7oc7ccccc78)n6)cc5)cc4)c3c2)cc1 ; c1cc(-c2cccc(-n3c4ccccc4c4ccccc43)c2)cc(-c2nc(-c3ccc4c(ccc5ccccc54)c3)nc(-c3cncc4c3oc3ccccc34)n2)c1]

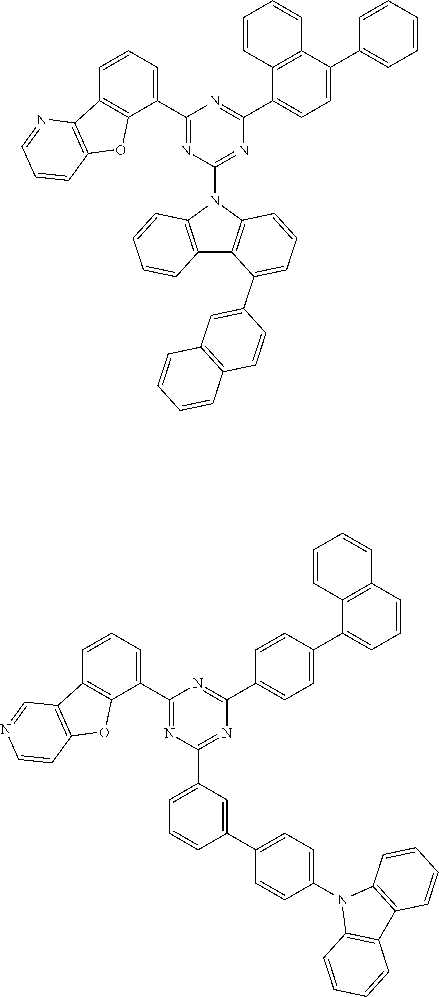 [SMILES: c1ccc(-c2ccc(-c3nc(-c4cccc5c4oc4cccnc45)nc(-n4c5ccccc5c5c(-c6ccc7ccccc7c6)cccc54)n3)c3ccccc23)cc1 ; c1cc(-c2ccc(-n3c4ccccc4c4ccccc43)cc2)cc(-c2nc(-c3ccc(-c4cccc5ccccc45)cc3)nc(-c3cccc4c3oc3ccncc34)n2)c1]

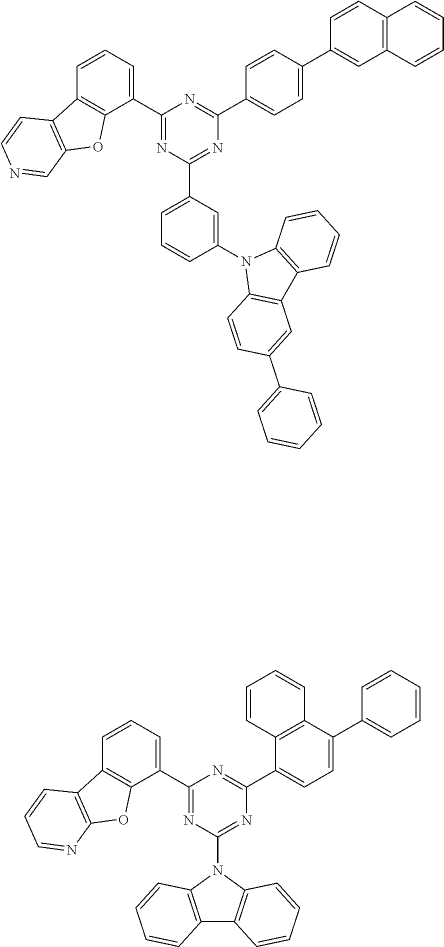 [SMILES: c1ccc(-c2ccc3c(c2)c2ccccc2n3-c2cccc(-c3nc(-c4ccc(-c5ccc6ccccc6c5)cc4)nc(-c4cccc5c4oc4cnccc45)n3)c2)cc1 ; c1ccc(-c2ccc(-c3nc(-c4cccc5c4oc4ncccc45)nc(-n4c5ccccc5c5ccccc54)n3)c3ccccc23)cc1]

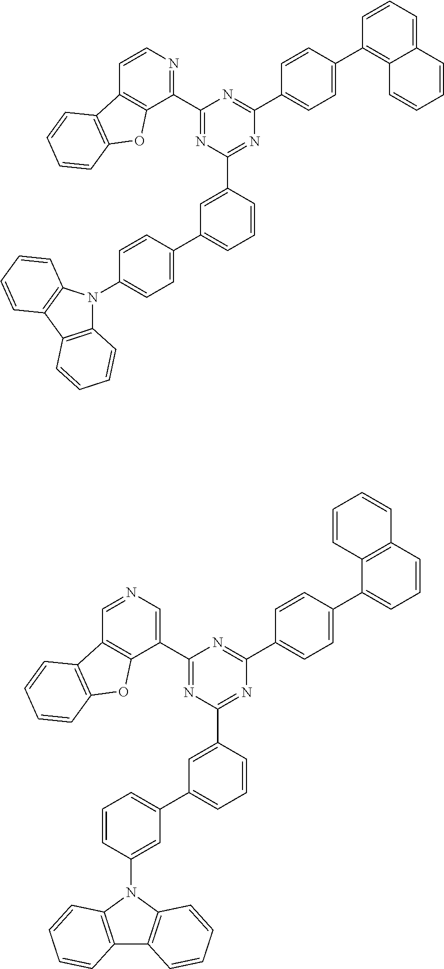 [SMILES: c1cc(-c2ccc(-n3c4ccccc4c4ccccc43)cc2)cc(-c2nc(-c3ccc(-c4cccc5ccccc45)cc3)nc(-c3nccc4c3oc3ccccc34)n2)c1 ; c1cc(-c2cccc(-n3c4ccccc4c4ccccc43)c2)cc(-c2nc(-c3ccc(-c4cccc5ccccc45)cc3)nc(-c3cncc4c3oc3ccccc34)n2)c1]

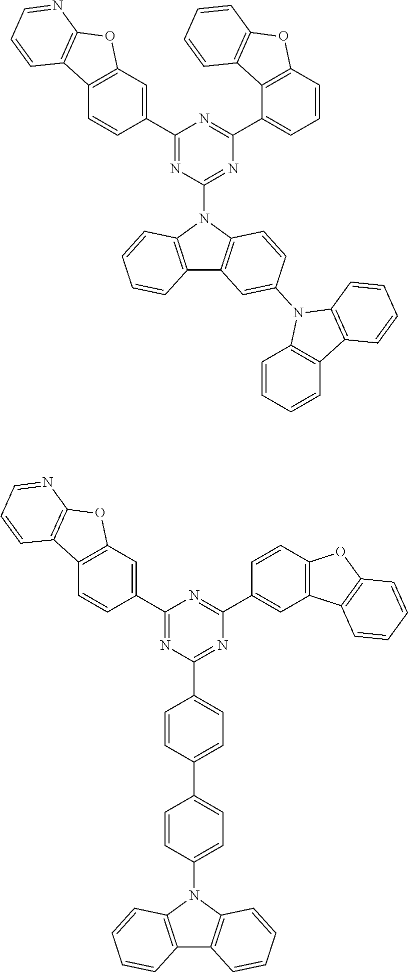 [SMILES: c1ccc2c(c1)oc1cccc(-c3nc(-c4ccc5c(c4)oc4ncccc45)nc(-n4c5ccccc5c5cc(-n6c7ccccc7c7ccccc76)ccc54)n3)c12 ; c1ccc2c(c1)oc1ccc(-c3nc(-c4ccc(-c5ccc(-n6c7ccccc7c7ccccc76)cc5)cc4)nc(-c4ccc5c(c4)oc4ncccc45)n3)cc12]

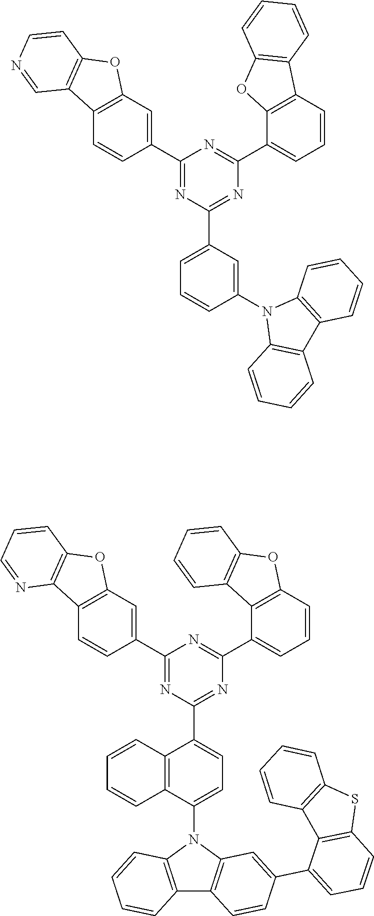 [SMILES: c1cc(-c2nc(-c3ccc4c(c3)oc3ccncc34)nc(-c3cccc4c3oc3ccccc34)n2)cc(-n2c3ccccc3c3ccccc32)c1 ; c1ccc2c(c1)oc1cccc(-c3nc(-c4ccc5c(c4)oc4cccnc45)nc(-c4ccc(-n5c6ccccc6c6ccc(-c7cccc8sc9ccccc9c78)cc65)c5ccccc45)n3)c12]

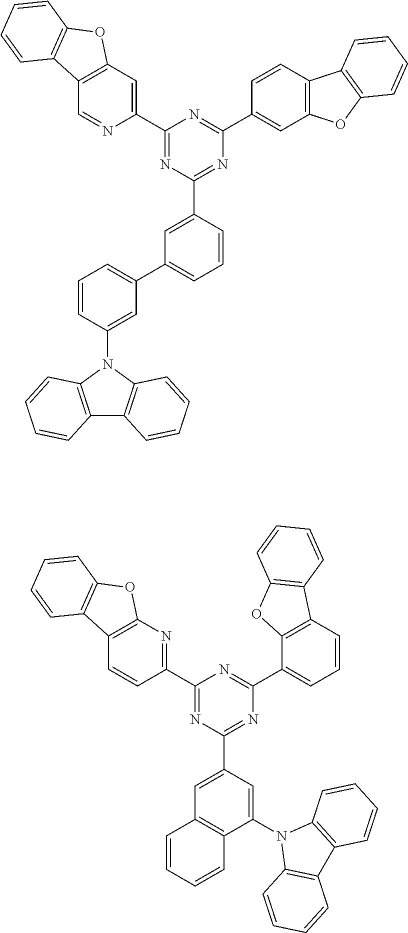 [SMILES: c1cc(-c2cccc(-n3c4ccccc4c4ccccc43)c2)cc(-c2nc(-c3ccc4c(c3)oc3ccccc34)nc(-c3cc4oc5ccccc5c4cn3)n2)c1 ; c1ccc2c(-n3c4ccccc4c4ccccc43)cc(-c3nc(-c4ccc5c(n4)oc4ccccc45)nc(-c4cccc5c4oc4ccccc45)n3)cc2c1]

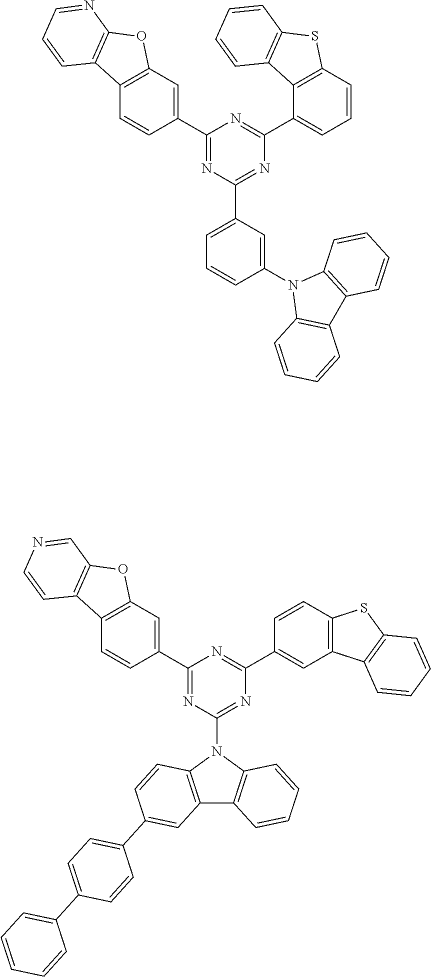 [SMILES: c1cc(-c2nc(-c3ccc4c(c3)oc3ncccc34)nc(-c3cccc4sc5ccccc5c34)n2)cc(-n2c3ccccc3c3ccccc32)c1 ; c1ccc(-c2ccc(-c3ccc4c(c3)c3ccccc3n4-c3nc(-c4ccc5c(c4)oc4cnccc45)nc(-c4ccc5sc6ccccc6c5c4)n3)cc2)cc1]

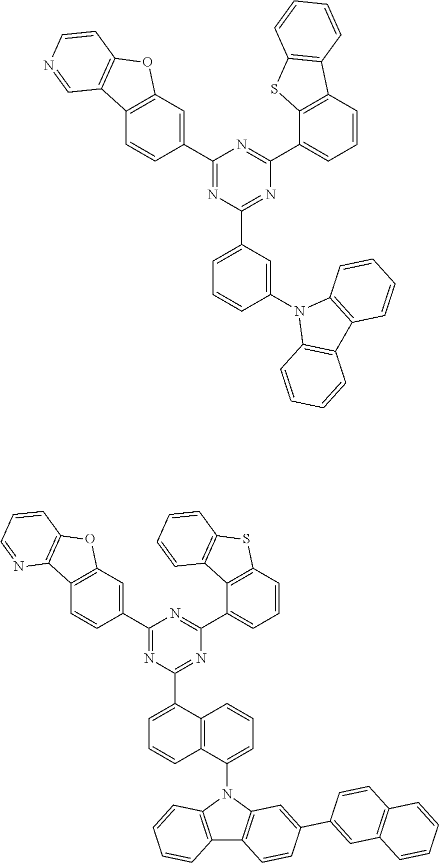 [SMILES: c1cc(-c2nc(-c3ccc4c(c3)oc3ccncc34)nc(-c3cccc4c3sc3ccccc34)n2)cc(-n2c3ccccc3c3ccccc32)c1 ; c1ccc2cc(-c3ccc4c5ccccc5n(-c5cccc6c(-c7nc(-c8ccc9c(c8)oc8cccnc89)nc(-c8cccc9sc%10ccccc%10c89)n7)cccc56)c4c3)ccc2c1]

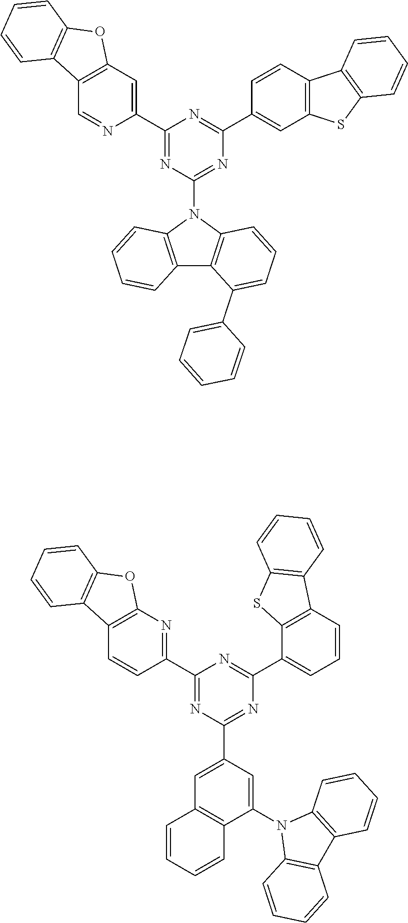 [SMILES: c1ccc(-c2cccc3c2c2ccccc2n3-c2nc(-c3ccc4c(c3)sc3ccccc34)nc(-c3cc4oc5ccccc5c4cn3)n2)cc1 ; c1ccc2c(-n3c4ccccc4c4ccccc43)cc(-c3nc(-c4ccc5c(n4)oc4ccccc45)nc(-c4cccc5c4sc4ccccc45)n3)cc2c1]

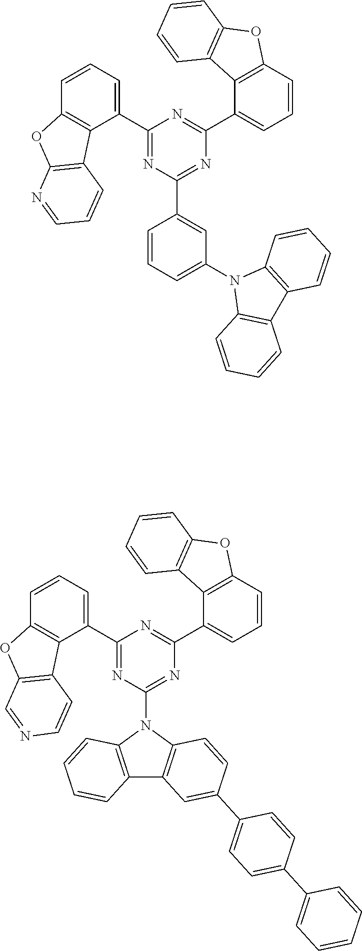 [SMILES: c1cc(-c2nc(-c3cccc4oc5ccccc5c34)nc(-c3cccc4oc5ncccc5c34)n2)cc(-n2c3ccccc3c3ccccc32)c1 ; c1ccc(-c2ccc(-c3ccc4c(c3)c3ccccc3n4-c3nc(-c4cccc5oc6ccccc6c45)nc(-c4cccc5oc6cnccc6c45)n3)cc2)cc1]

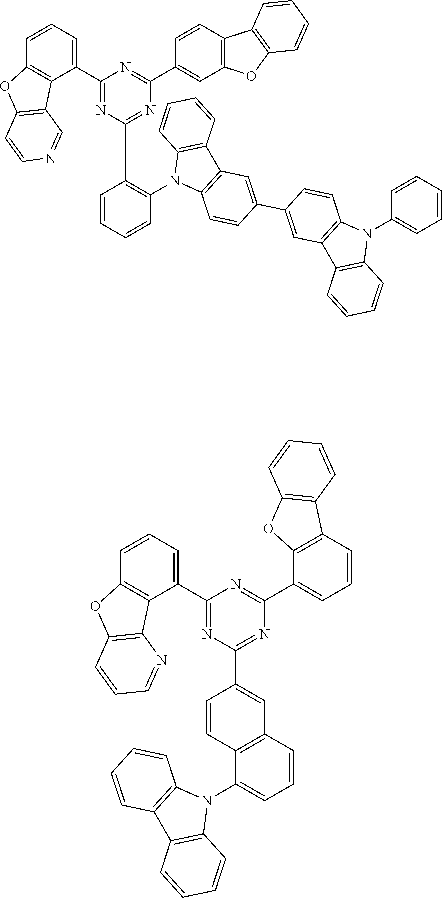 [SMILES: c1ccc(-n2c3ccccc3c3cc(-c4ccc5c(c4)c4ccccc4n5-c4ccccc4-c4nc(-c5ccc6c(c5)oc5ccccc56)nc(-c5cccc6oc7ccncc7c56)n4)ccc32)cc1 ; c1cc(-n2c3ccccc3c3ccccc32)c2ccc(-c3nc(-c4cccc5c4oc4ccccc45)nc(-c4cccc5oc6cccnc6c45)n3)cc2c1]

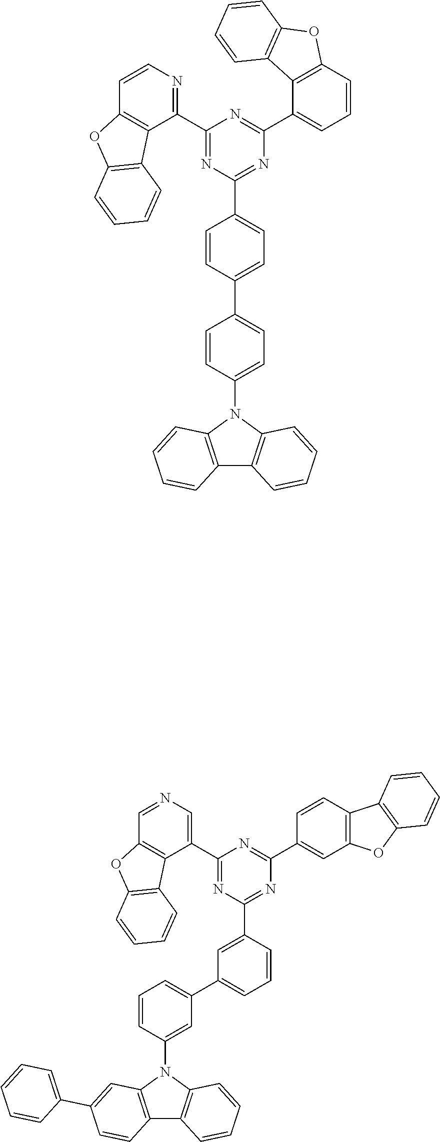 [SMILES: c1ccc2c(c1)oc1cccc(-c3nc(-c4ccc(-c5ccc(-n6c7ccccc7c7ccccc76)cc5)cc4)nc(-c4nccc5oc6ccccc6c45)n3)c12 ; c1ccc(-c2ccc3c4ccccc4n(-c4cccc(-c5cccc(-c6nc(-c7ccc8c(c7)oc7ccccc78)nc(-c7cncc8oc9ccccc9c78)n6)c5)c4)c3c2)cc1]

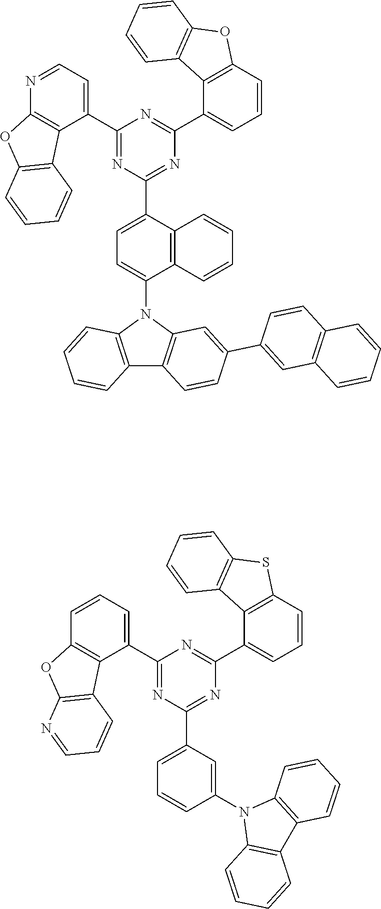 [SMILES: c1ccc2cc(-c3ccc4c5ccccc5n(-c5ccc(-c6nc(-c7cccc8oc9ccccc9c78)nc(-c7ccnc8oc9ccccc9c78)n6)c6ccccc56)c4c3)ccc2c1 ; c1cc(-c2nc(-c3cccc4oc5ncccc5c34)nc(-c3cccc4sc5ccccc5c34)n2)cc(-n2c3ccccc3c3ccccc32)c1]

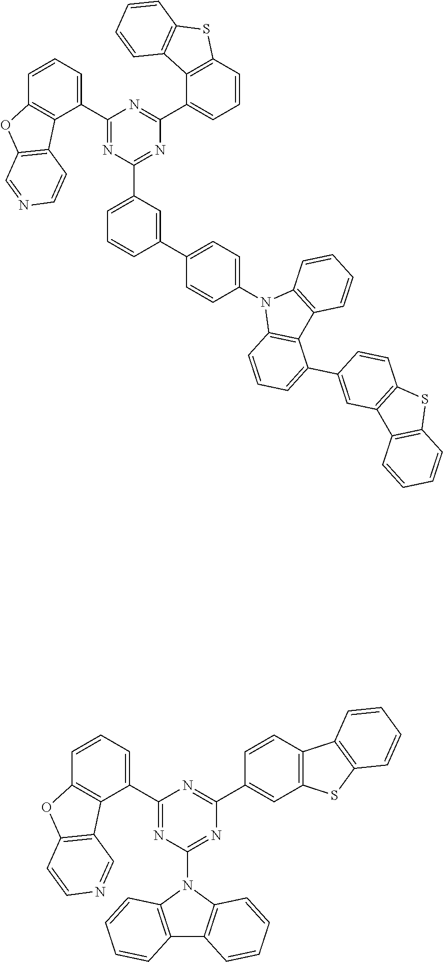 [SMILES: c1cc(-c2ccc(-n3c4ccccc4c4c(-c5ccc6sc7ccccc7c6c5)cccc43)cc2)cc(-c2nc(-c3cccc4oc5cnccc5c34)nc(-c3cccc4sc5ccccc5c34)n2)c1 ; c1cc(-c2nc(-c3ccc4c(c3)sc3ccccc34)nc(-n3c4ccccc4c4ccccc43)n2)c2c(c1)oc1ccncc12]

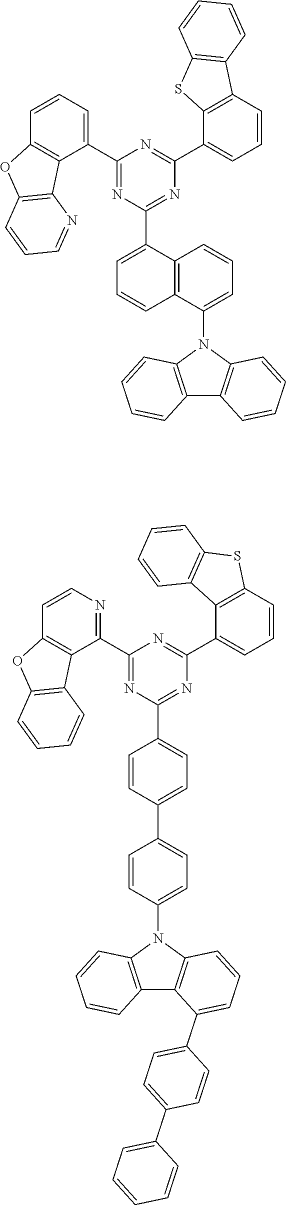 [SMILES: c1cnc2c(c1)oc1cccc(-c3nc(-c4cccc5c(-n6c7ccccc7c7ccccc76)cccc45)nc(-c4cccc5c4sc4ccccc45)n3)c12 ; c1ccc(-c2ccc(-c3cccc4c3c3ccccc3n4-c3ccc(-c4ccc(-c5nc(-c6nccc7oc8ccccc8c67)nc(-c6cccc7sc8ccccc8c67)n5)cc4)cc3)cc2)cc1]

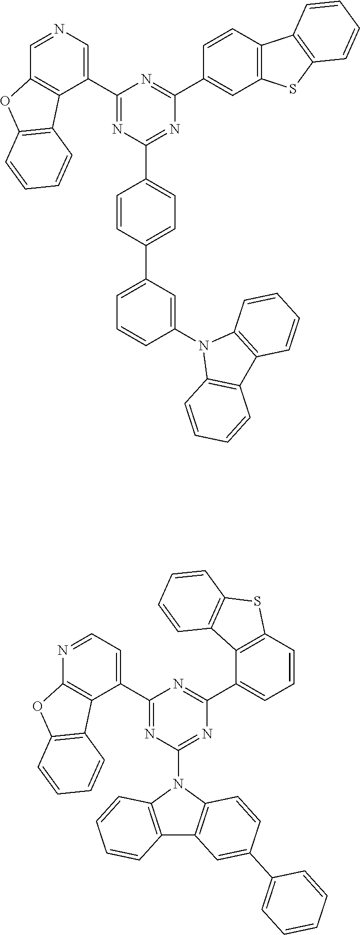 [SMILES: c1cc(-c2ccc(-c3nc(-c4ccc5c(c4)sc4ccccc45)nc(-c4cncc5oc6ccccc6c45)n3)cc2)cc(-n2c3ccccc3c3ccccc32)c1 ; c1ccc(-c2ccc3c(c2)c2ccccc2n3-c2nc(-c3ccnc4oc5ccccc5c34)nc(-c3cccc4sc5ccccc5c34)n2)cc1]

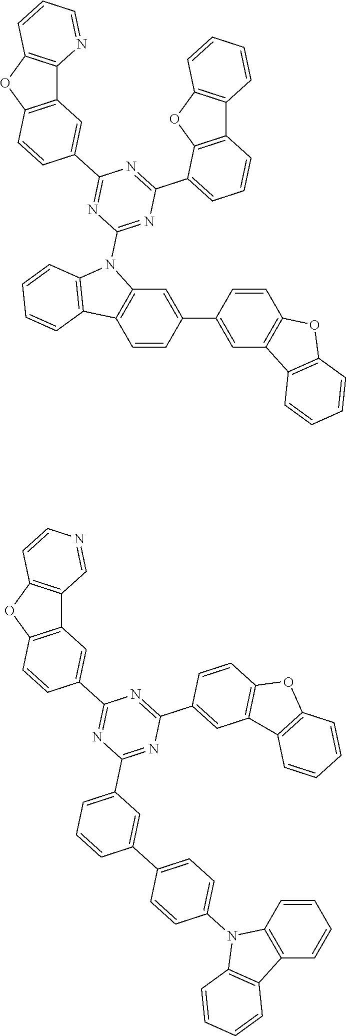 [SMILES: c1ccc2c(c1)oc1ccc(-c3ccc4c5ccccc5n(-c5nc(-c6ccc7oc8cccnc8c7c6)nc(-c6cccc7c6oc6ccccc67)n5)c4c3)cc12 ; c1cc(-c2ccc(-n3c4ccccc4c4ccccc43)cc2)cc(-c2nc(-c3ccc4oc5ccccc5c4c3)nc(-c3ccc4oc5ccncc5c4c3)n2)c1]

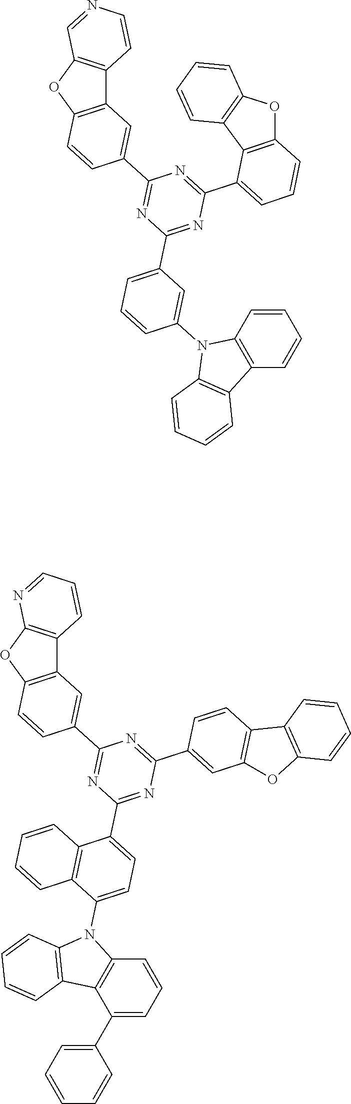 [SMILES: c1cc(-c2nc(-c3ccc4oc5cnccc5c4c3)nc(-c3cccc4oc5ccccc5c34)n2)cc(-n2c3ccccc3c3ccccc32)c1 ; c1ccc(-c2cccc3c2c2ccccc2n3-c2ccc(-c3nc(-c4ccc5c(c4)oc4ccccc45)nc(-c4ccc5oc6ncccc6c5c4)n3)c3ccccc23)cc1]

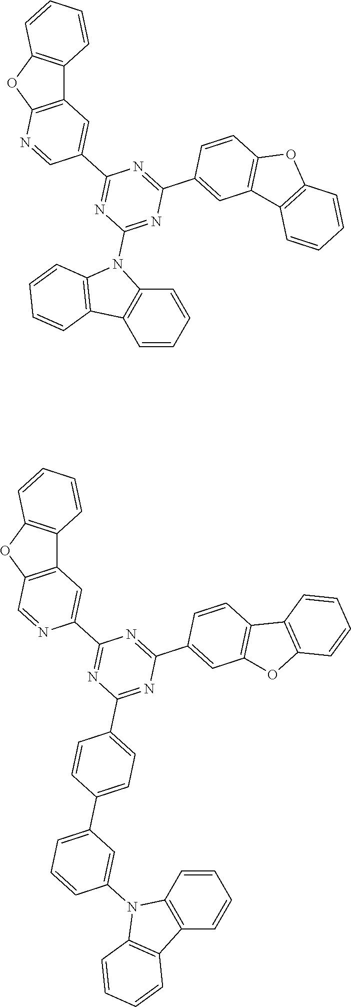 [SMILES: c1ccc2c(c1)oc1ccc(-c3nc(-c4cnc5oc6ccccc6c5c4)nc(-n4c5ccccc5c5ccccc54)n3)cc12 ; c1cc(-c2ccc(-c3nc(-c4ccc5c(c4)oc4ccccc45)nc(-c4cc5c(cn4)oc4ccccc45)n3)cc2)cc(-n2c3ccccc3c3ccccc32)c1]

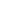

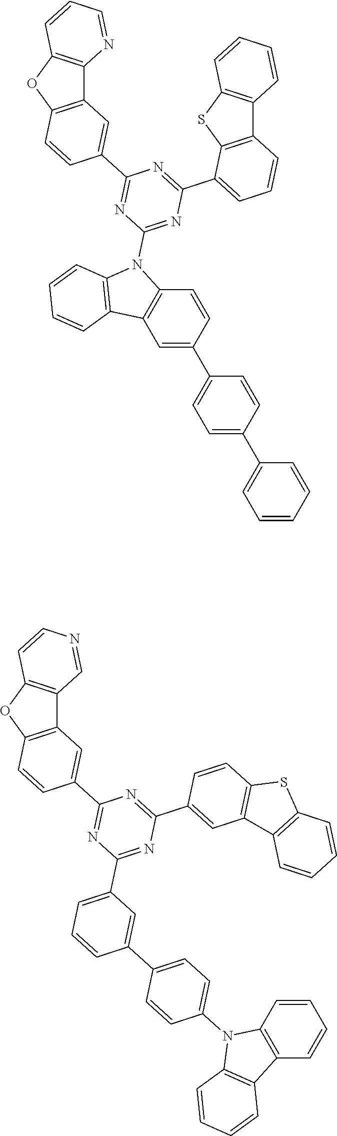 [SMILES: c1ccc(-c2ccc(-c3ccc4c(c3)c3ccccc3n4-c3nc(-c4ccc5oc6cccnc6c5c4)nc(-c4cccc5c4sc4ccccc45)n3)cc2)cc1 ; c1cc(-c2ccc(-n3c4ccccc4c4ccccc43)cc2)cc(-c2nc(-c3ccc4oc5ccncc5c4c3)nc(-c3ccc4sc5ccccc5c4c3)n2)c1]

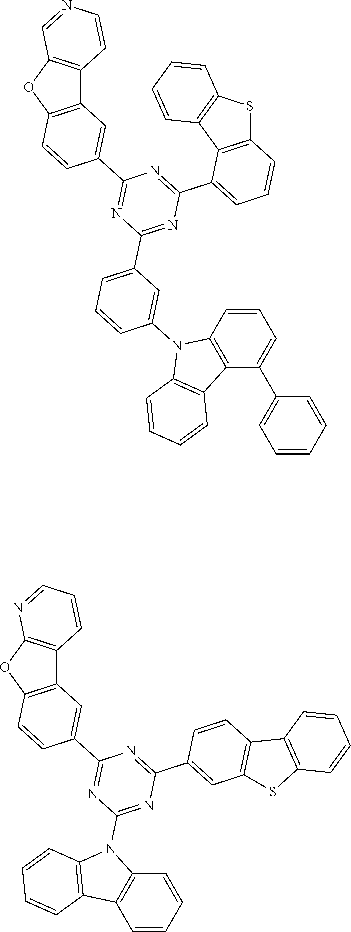 [SMILES: c1ccc(-c2cccc3c2c2ccccc2n3-c2cccc(-c3nc(-c4ccc5oc6cnccc6c5c4)nc(-c4cccc5sc6ccccc6c45)n3)c2)cc1 ; c1ccc2c(c1)sc1cc(-c3nc(-c4ccc5oc6ncccc6c5c4)nc(-n4c5ccccc5c5ccccc54)n3)ccc12]

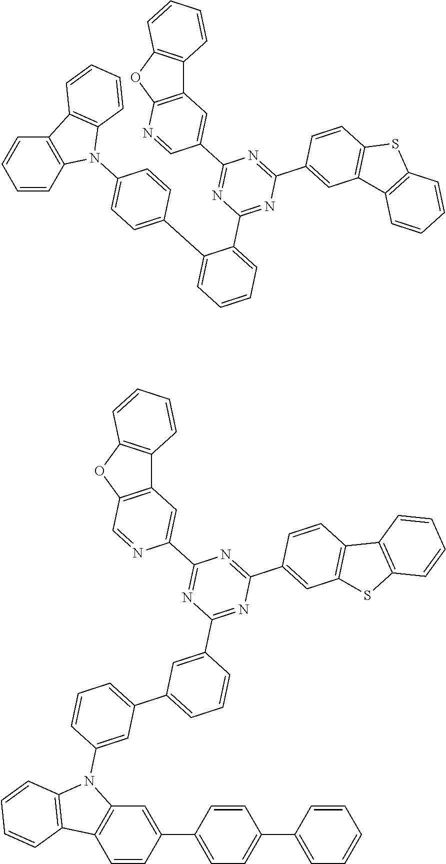 [SMILES: c1ccc(-c2nc(-c3cnc4oc5ccccc5c4c3)nc(-c3ccc4sc5ccccc5c4c3)n2)c(-c2ccc(-n3c4ccccc4c4ccccc43)cc2)c1 ; c1ccc(-c2ccc(-c3ccc4c5ccccc5n(-c5cccc(-c6cccc(-c7nc(-c8ccc9c(c8)sc8ccccc89)nc(-c8cc9c(cn8)oc8ccccc89)n7)c6)c5)c4c3)cc2)cc1]

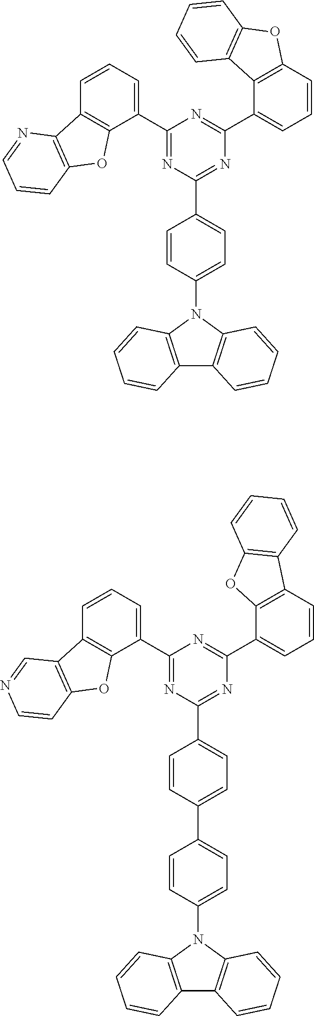 [SMILES: c1ccc2c(c1)oc1cccc(-c3nc(-c4ccc(-n5c6ccccc6c6ccccc65)cc4)nc(-c4cccc5c4oc4cccnc45)n3)c12 ; c1ccc2c(c1)oc1c(-c3nc(-c4ccc(-c5ccc(-n6c7ccccc7c7ccccc76)cc5)cc4)nc(-c4cccc5c4oc4ccncc45)n3)cccc12]

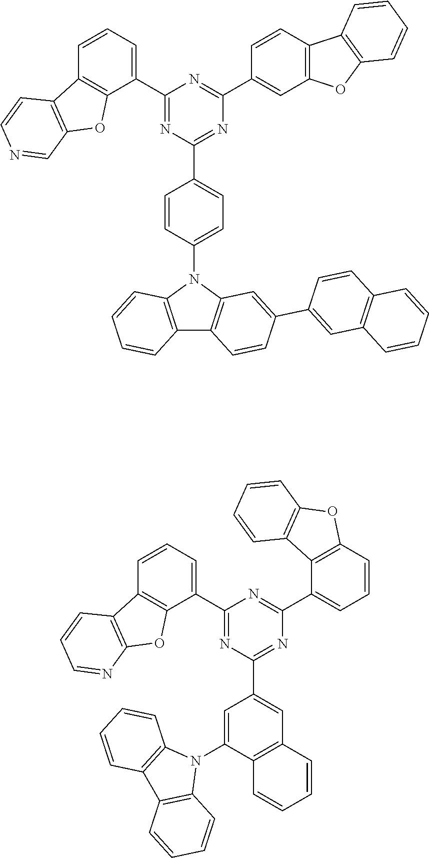 [SMILES: c1ccc2cc(-c3ccc4c5ccccc5n(-c5ccc(-c6nc(-c7ccc8c(c7)oc7ccccc78)nc(-c7cccc8c7oc7cnccc78)n6)cc5)c4c3)ccc2c1 ; c1ccc2c(-n3c4ccccc4c4ccccc43)cc(-c3nc(-c4cccc5c4oc4ncccc45)nc(-c4cccc5oc6ccccc6c45)n3)cc2c1]

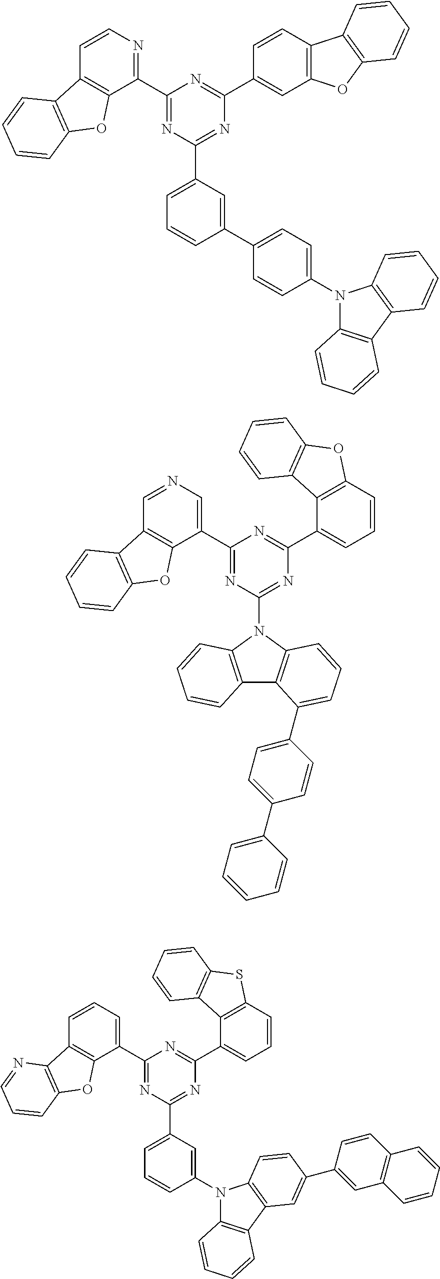 [SMILES: c1cc(-c2ccc(-n3c4ccccc4c4ccccc43)cc2)cc(-c2nc(-c3ccc4c(c3)oc3ccccc34)nc(-c3nccc4c3oc3ccccc34)n2)c1 ; c1ccc(-c2ccc(-c3cccc4c3c3ccccc3n4-c3nc(-c4cncc5c4oc4ccccc45)nc(-c4cccc5oc6ccccc6c45)n3)cc2)cc1 ; c1cc(-c2nc(-c3cccc4c3oc3cccnc34)nc(-c3cccc4sc5ccccc5c34)n2)cc(-n2c3ccccc3c3cc(-c4ccc5ccccc5c4)ccc32)c1]

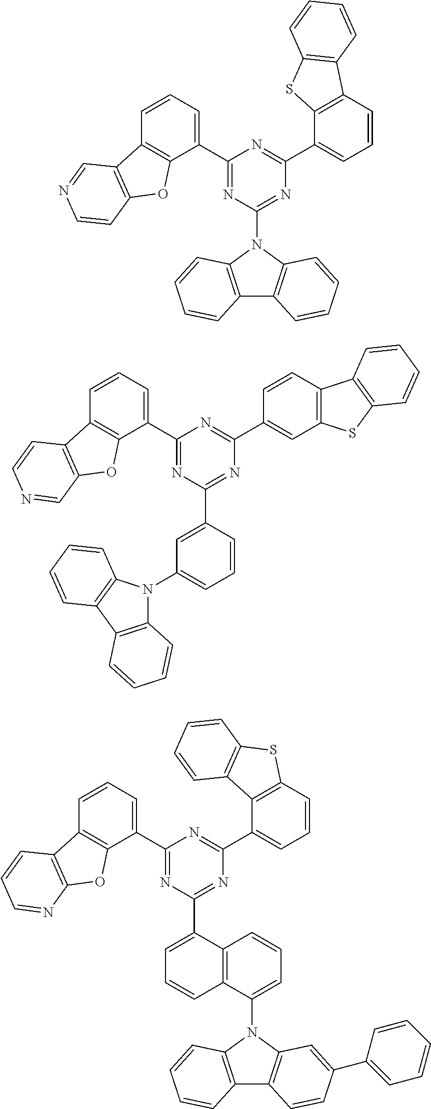 [SMILES: c1ccc2c(c1)sc1c(-c3nc(-c4cccc5c4oc4ccncc45)nc(-n4c5ccccc5c5ccccc54)n3)cccc12 ; c1cc(-c2nc(-c3ccc4c(c3)sc3ccccc34)nc(-c3cccc4c3oc3cnccc34)n2)cc(-n2c3ccccc3c3ccccc32)c1 ; c1ccc(-c2ccc3c4ccccc4n(-c4cccc5c(-c6nc(-c7cccc8c7oc7ncccc78)nc(-c7cccc8sc9ccccc9c78)n6)cccc45)c3c2)cc1]

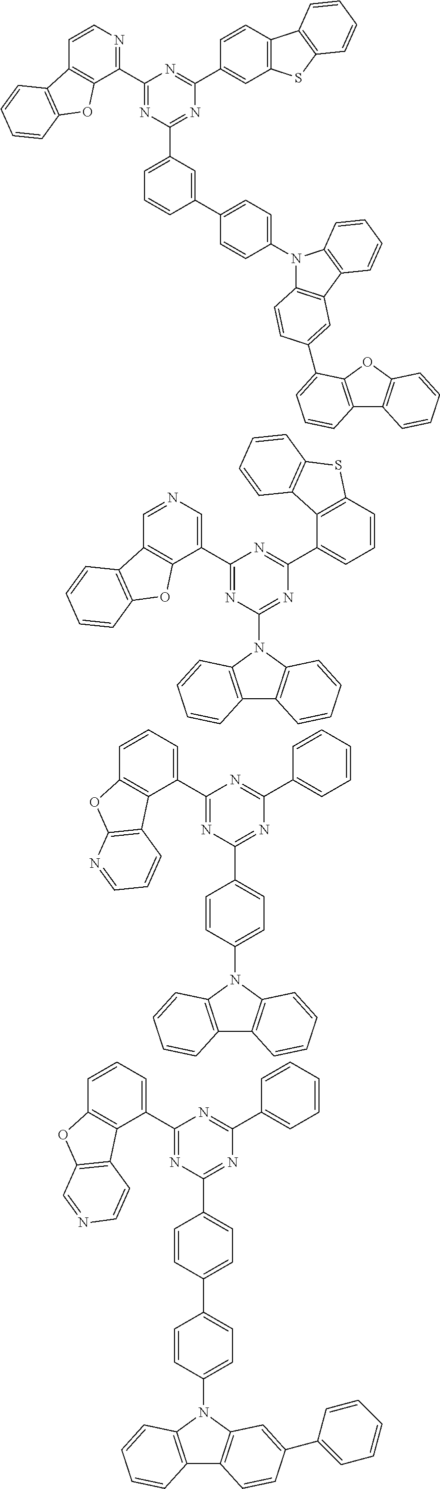

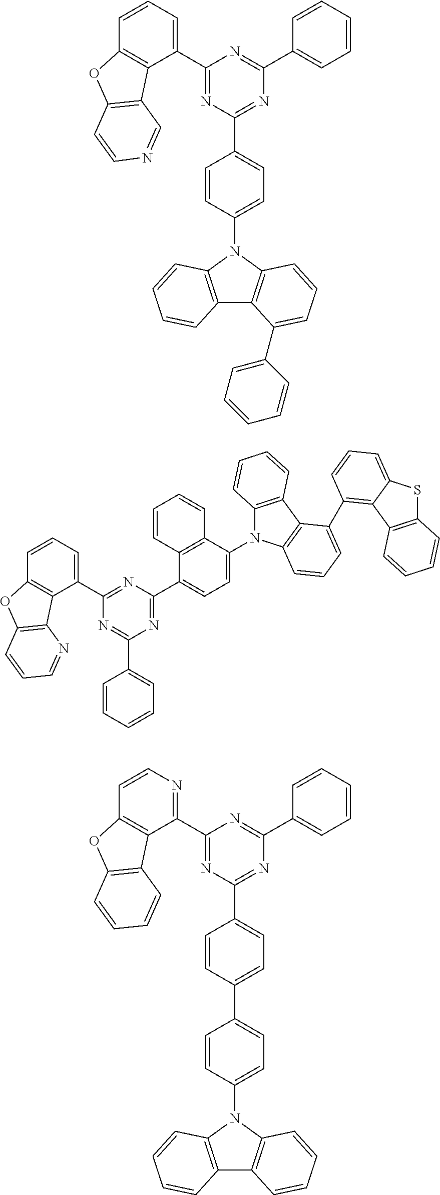

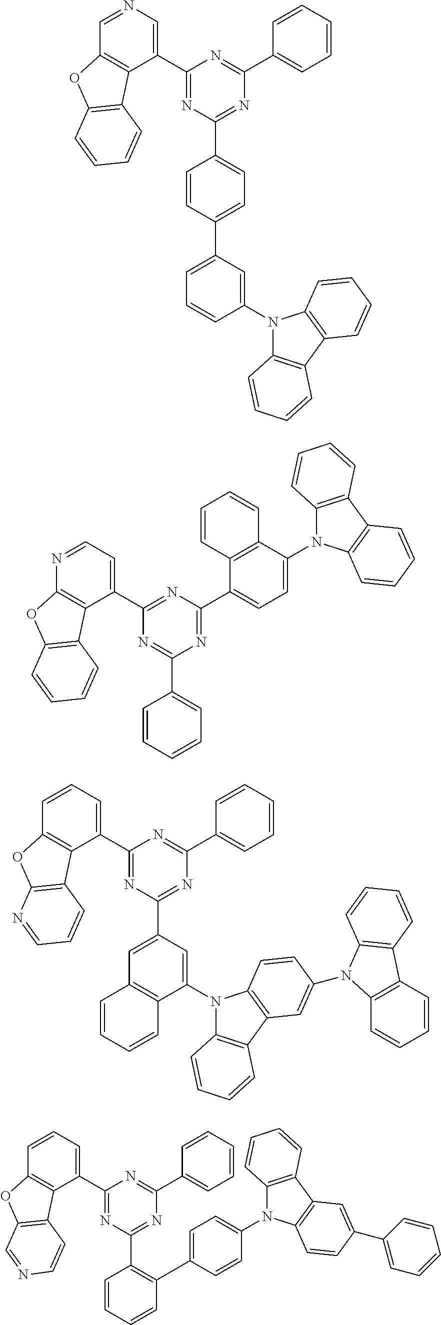

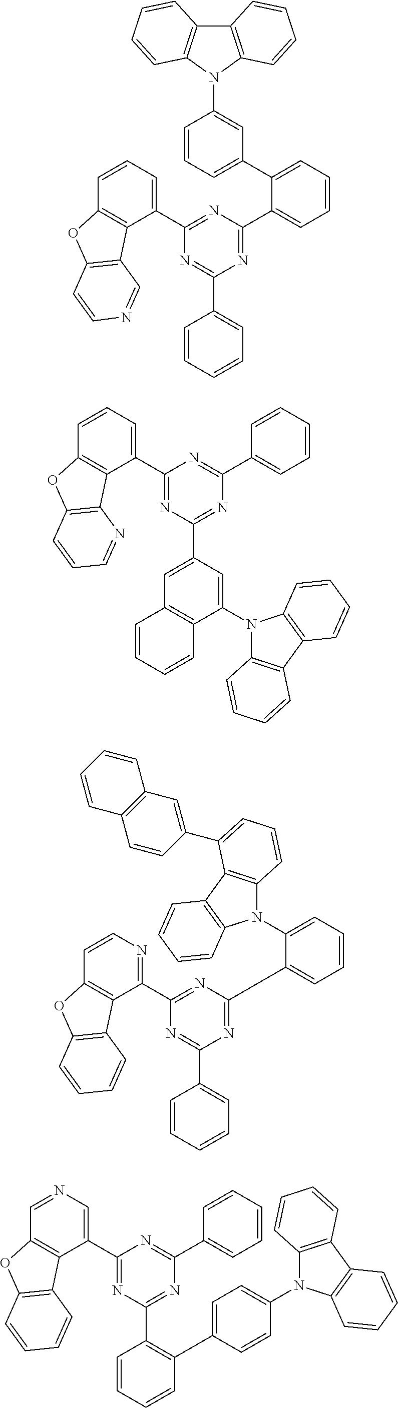 [SMILES: c1ccc(-c2nc(-c3ccccc3-c3cccc(-n4c5ccccc5c5ccccc54)c3)nc(-c3cccc4oc5ccncc5c34)n2)cc1 ; c1ccc(-c2nc(-c3cc(-n4c5ccccc5c5ccccc54)c4ccccc4c3)nc(-c3cccc4oc5cccnc5c34)n2)cc1 ; c1ccc(-c2nc(-c3ccccc3-n3c4ccccc4c4c(-c5ccc6ccccc6c5)cccc43)nc(-c3nccc4oc5ccccc5c34)n2)cc1 ; c1ccc(-c2nc(-c3ccccc3-c3ccc(-n4c5ccccc5c5ccccc54)cc3)nc(-c3cncc4oc5ccccc5c34)n2)cc1]

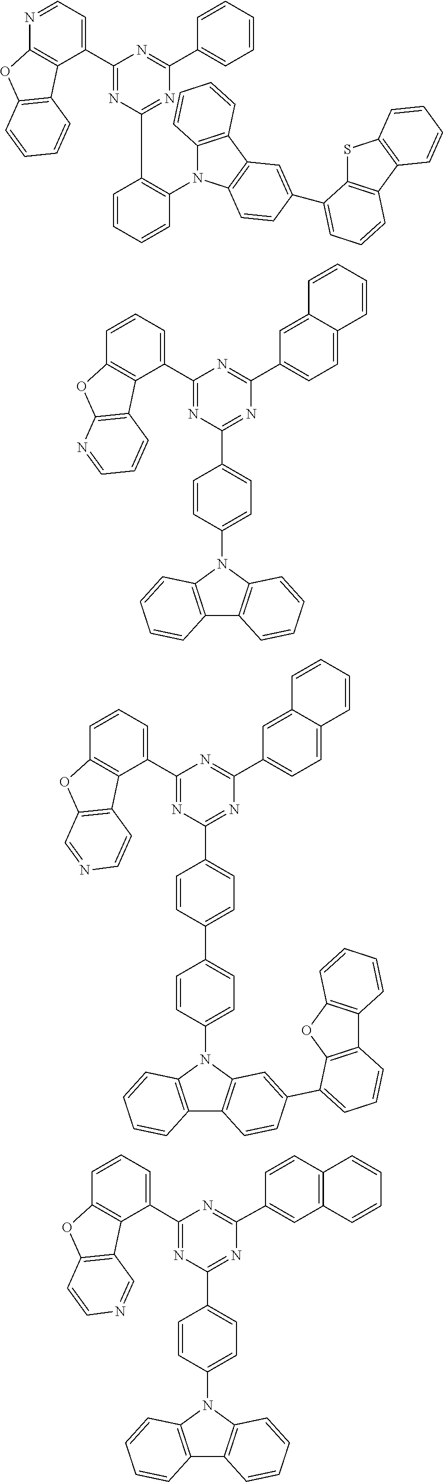

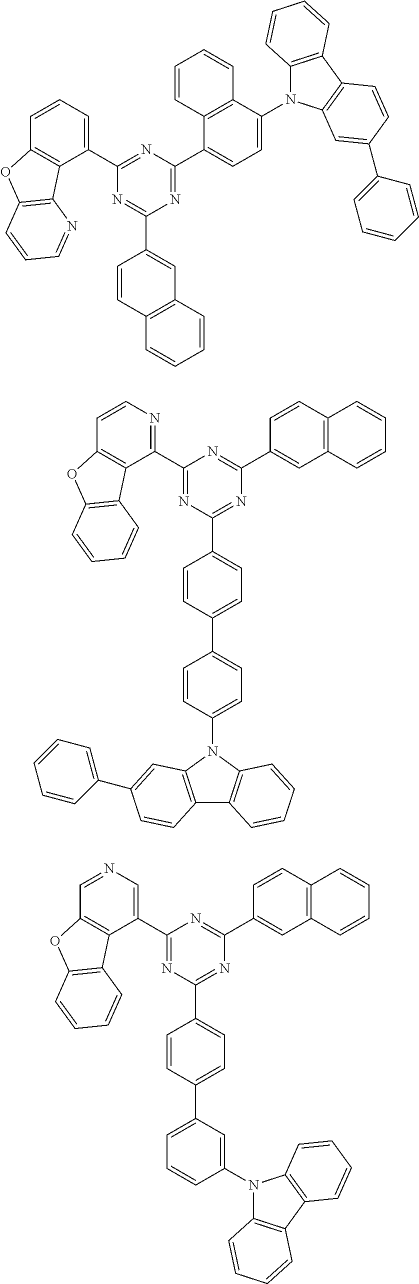 [SMILES: c1ccc(-c2ccc3c4ccccc4n(-c4ccc(-c5nc(-c6ccc7ccccc7c6)nc(-c6cccc7oc8cccnc8c67)n5)c5ccccc45)c3c2)cc1 ; c1ccc(-c2ccc3c4ccccc4n(-c4ccc(-c5ccc(-c6nc(-c7ccc8ccccc8c7)nc(-c7nccc8oc9ccccc9c78)n6)cc5)cc4)c3c2)cc1 ; c1cc(-c2ccc(-c3nc(-c4ccc5ccccc5c4)nc(-c4cncc5oc6ccccc6c45)n3)cc2)cc(-n2c3ccccc3c3ccccc32)c1]

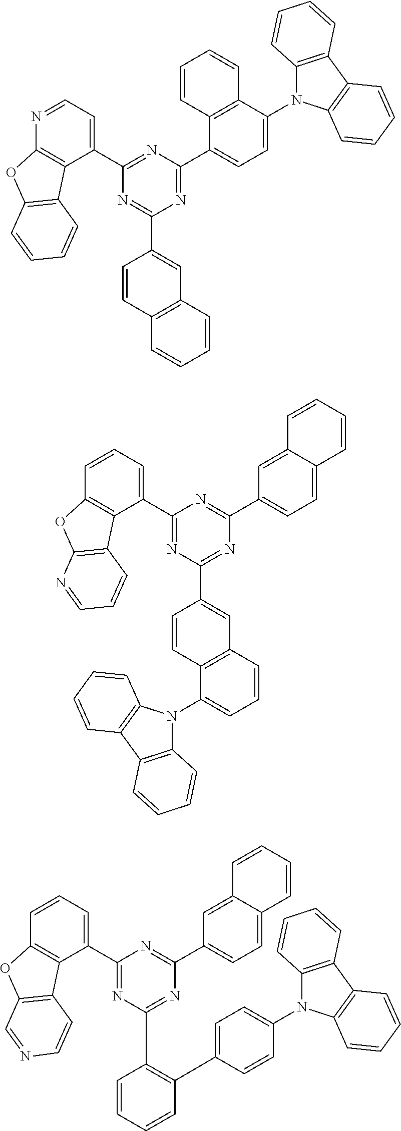 [SMILES: c1ccc2cc(-c3nc(-c4ccc(-n5c6ccccc6c6ccccc65)c5ccccc45)nc(-c4ccnc5oc6ccccc6c45)n3)ccc2c1 ; c1ccc2cc(-c3nc(-c4ccc5c(-n6c7ccccc7c7ccccc76)cccc5c4)nc(-c4cccc5oc6ncccc6c45)n3)ccc2c1 ; c1ccc(-c2nc(-c3ccc4ccccc4c3)nc(-c3cccc4oc5cnccc5c34)n2)c(-c2ccc(-n3c4ccccc4c4ccccc43)cc2)c1]

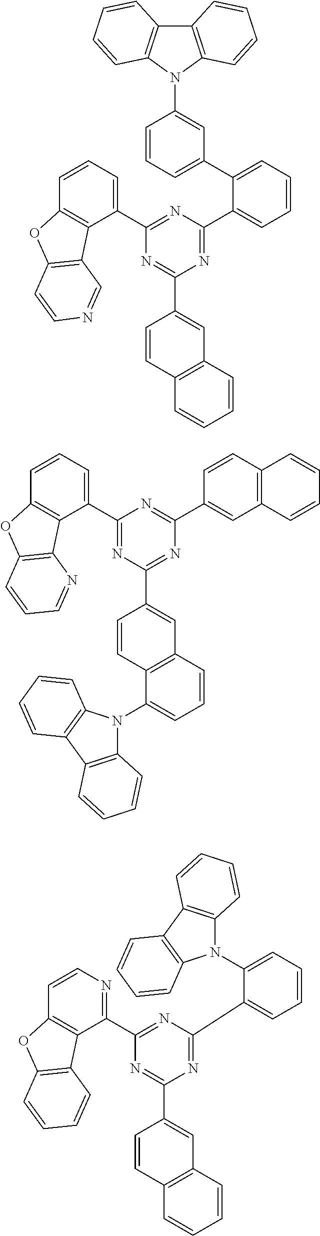

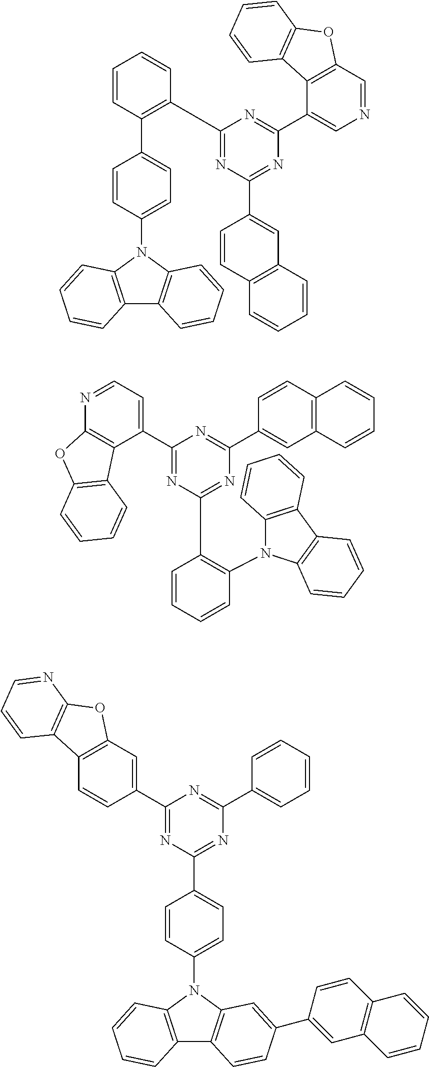 [SMILES: c1ccc(-c2nc(-c3ccc4ccccc4c3)nc(-c3cncc4oc5ccccc5c34)n2)c(-c2ccc(-n3c4ccccc4c4ccccc43)cc2)c1 ; c1ccc(-n2c3ccccc3c3ccccc32)c(-c2nc(-c3ccc4ccccc4c3)nc(-c3ccnc4oc5ccccc5c34)n2)c1 ; c1ccc(-c2nc(-c3ccc(-n4c5ccccc5c5ccc(-c6ccc7ccccc7c6)cc54)cc3)nc(-c3ccc4c(c3)oc3ncccc34)n2)cc1]

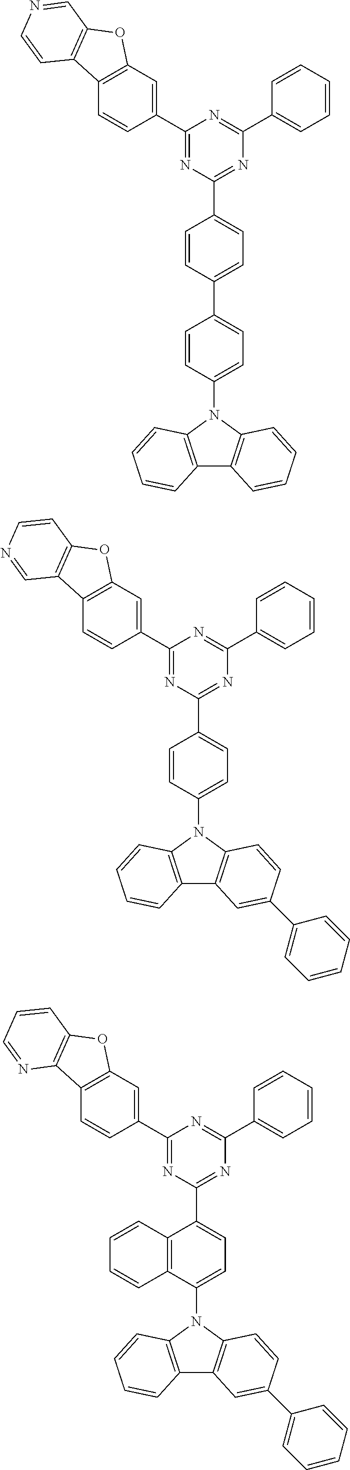 [SMILES: c1ccc(-c2nc(-c3ccc(-c4ccc(-n5c6ccccc6c6ccccc65)cc4)cc3)nc(-c3ccc4c(c3)oc3cnccc34)n2)cc1 ; c1ccc(-c2ccc3c(c2)c2ccccc2n3-c2ccc(-c3nc(-c4ccccc4)nc(-c4ccc5c(c4)oc4ccncc45)n3)cc2)cc1 ; c1ccc(-c2ccc3c(c2)c2ccccc2n3-c2ccc(-c3nc(-c4ccccc4)nc(-c4ccc5c(c4)oc4cccnc45)n3)c3ccccc23)cc1]

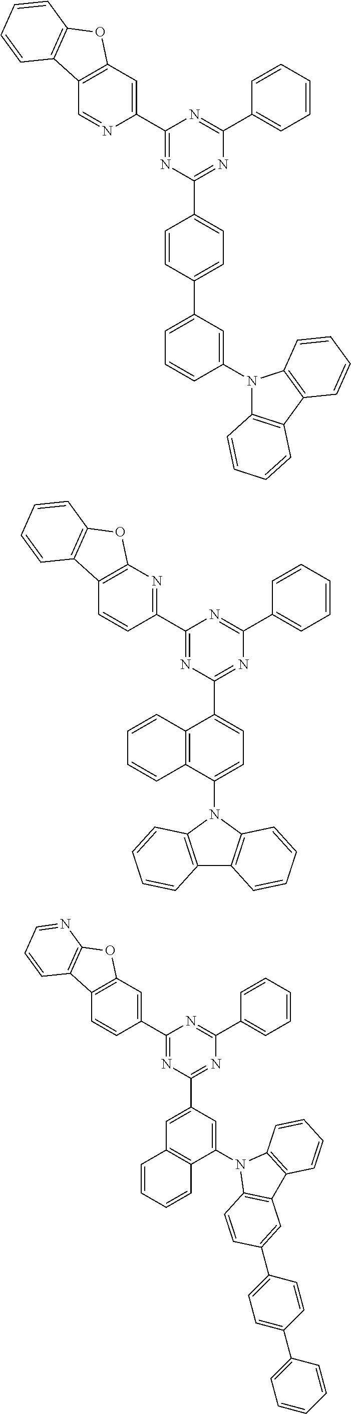 [SMILES: c1ccc(-c2nc(-c3ccc(-c4cccc(-n5c6ccccc6c6ccccc65)c4)cc3)nc(-c3cc4oc5ccccc5c4cn3)n2)cc1 ; c1ccc(-c2nc(-c3ccc4c(n3)oc3ccccc34)nc(-c3ccc(-n4c5ccccc5c5ccccc54)c4ccccc34)n2)cc1 ; c1ccc(-c2ccc(-c3ccc4c(c3)c3ccccc3n4-c3cc(-c4nc(-c5ccccc5)nc(-c5ccc6c(c5)oc5ncccc56)n4)cc4ccccc34)cc2)cc1]

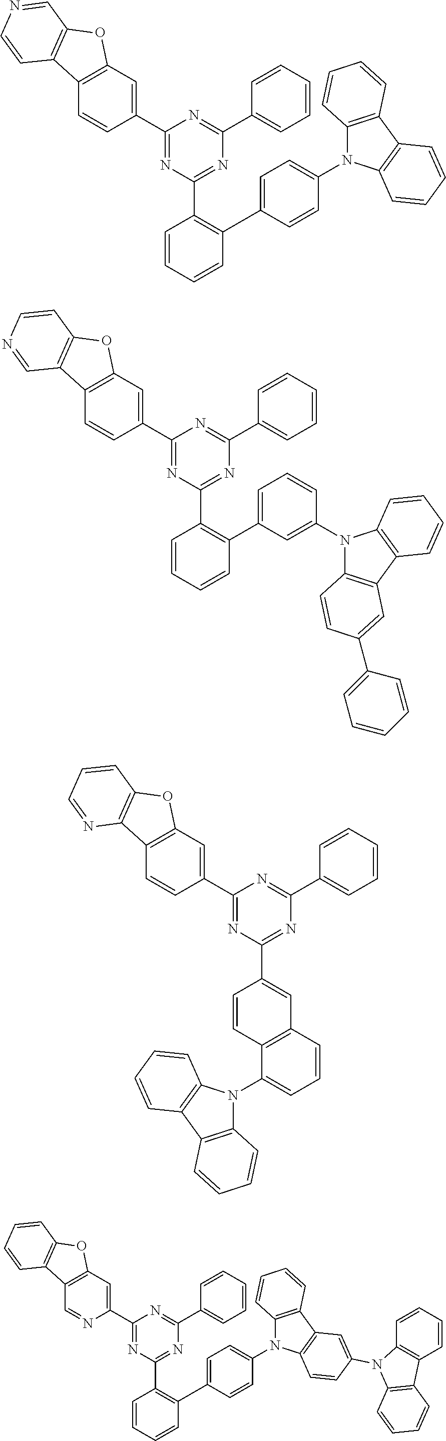 [SMILES: c1ccc(-c2nc(-c3ccc4c(c3)oc3cnccc34)nc(-c3ccccc3-c3ccc(-n4c5ccccc5c5ccccc54)cc3)n2)cc1 ; c1ccc(-c2ccc3c(c2)c2ccccc2n3-c2cccc(-c3ccccc3-c3nc(-c4ccccc4)nc(-c4ccc5c(c4)oc4ccncc45)n3)c2)cc1 ; c1ccc(-c2nc(-c3ccc4c(-n5c6ccccc6c6ccccc65)cccc4c3)nc(-c3ccc4c(c3)oc3cccnc34)n2)cc1 ; c1ccc(-c2nc(-c3cc4oc5ccccc5c4cn3)nc(-c3ccccc3-c3ccc(-n4c5ccccc5c5cc(-n6c7ccccc7c7ccccc76)ccc54)cc3)n2)cc1]

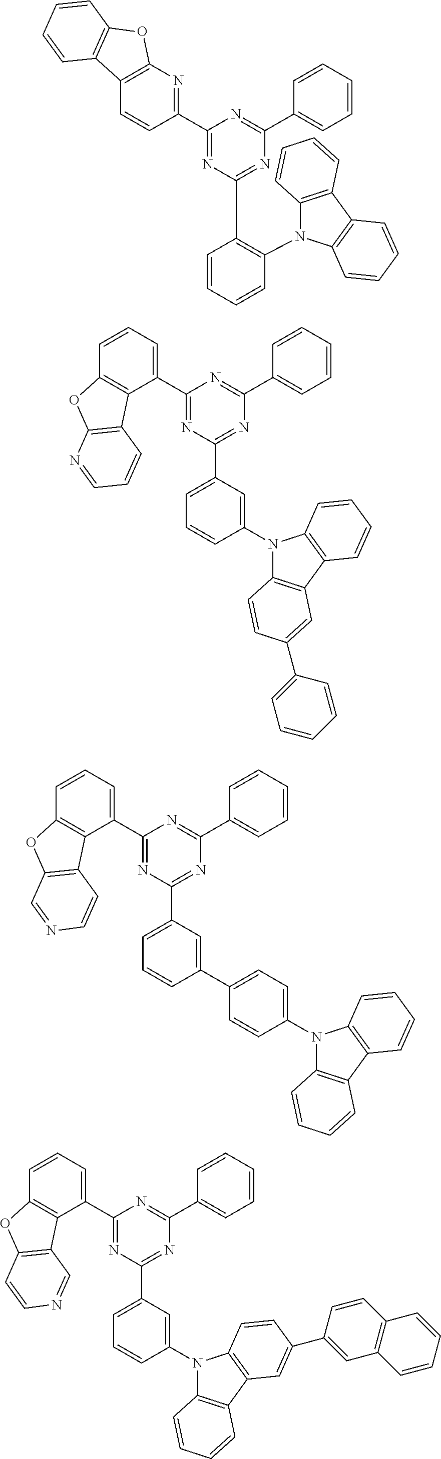

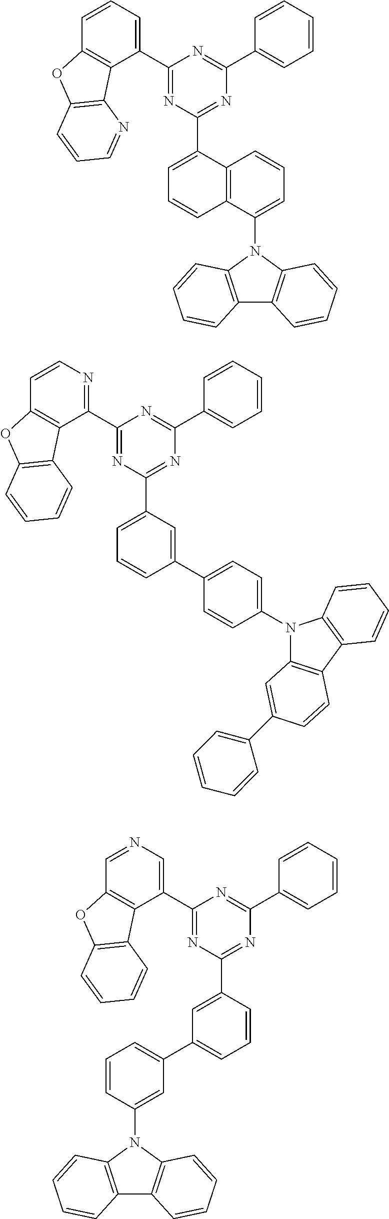

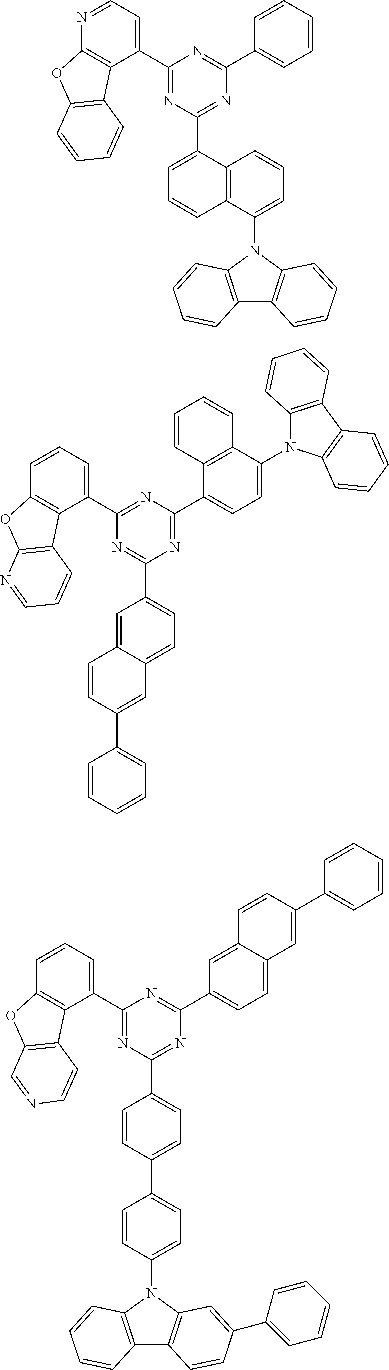

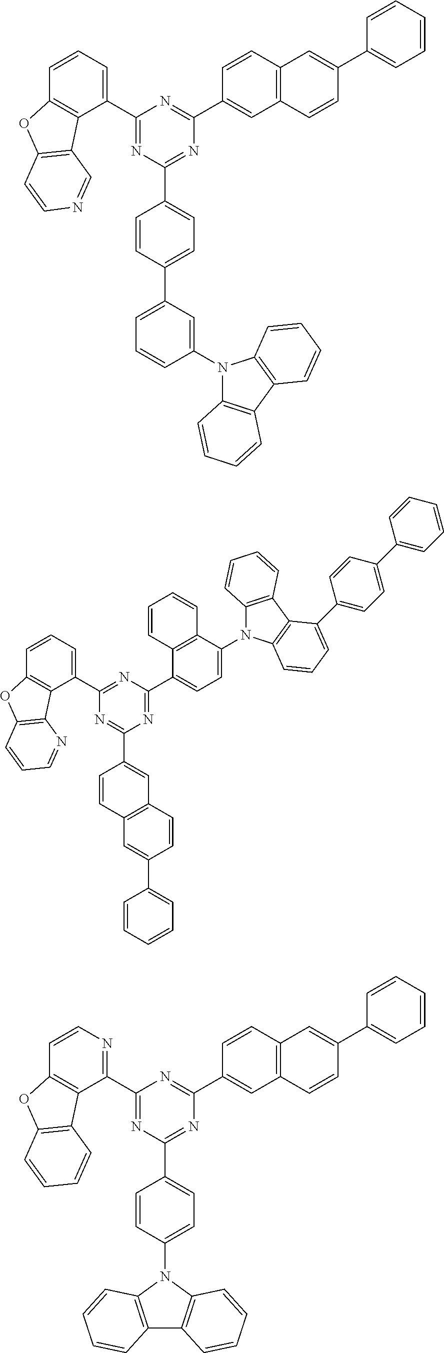 [SMILES: c1ccc(-c2ccc3cc(-c4nc(-c5ccc(-c6cccc(-n7c8ccccc8c8ccccc87)c6)cc5)nc(-c5cccc6oc7ccncc7c56)n4)ccc3c2)cc1 ; c1ccc(-c2ccc(-c3cccc4c3c3ccccc3n4-c3ccc(-c4nc(-c5ccc6cc(-c7ccccc7)ccc6c5)nc(-c5cccc6oc7cccnc7c56)n4)c4ccccc34)cc2)cc1 ; c1ccc(-c2ccc3cc(-c4nc(-c5ccc(-n6c7ccccc7c7ccccc76)cc5)nc(-c5nccc6oc7ccccc7c56)n4)ccc3c2)cc1]

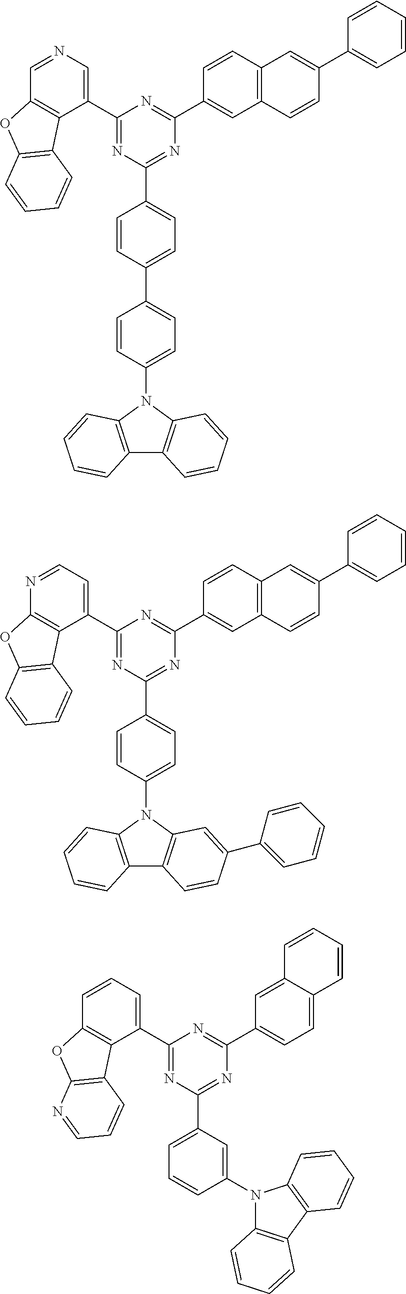 [SMILES: c1ccc(-c2ccc3cc(-c4nc(-c5ccc(-c6ccc(-n7c8ccccc8c8ccccc87)cc6)cc5)nc(-c5cncc6oc7ccccc7c56)n4)ccc3c2)cc1 ; c1ccc(-c2ccc3cc(-c4nc(-c5ccc(-n6c7ccccc7c7ccc(-c8ccccc8)cc76)cc5)nc(-c5ccnc6oc7ccccc7c56)n4)ccc3c2)cc1 ; c1cc(-c2nc(-c3ccc4ccccc4c3)nc(-c3cccc4oc5ncccc5c34)n2)cc(-n2c3ccccc3c3ccccc32)c1]

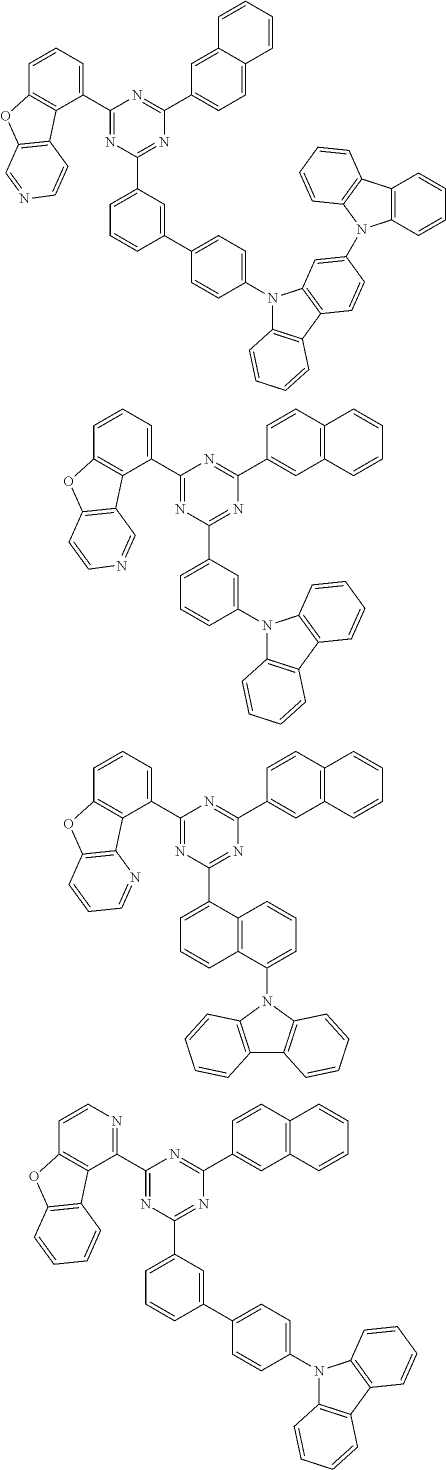

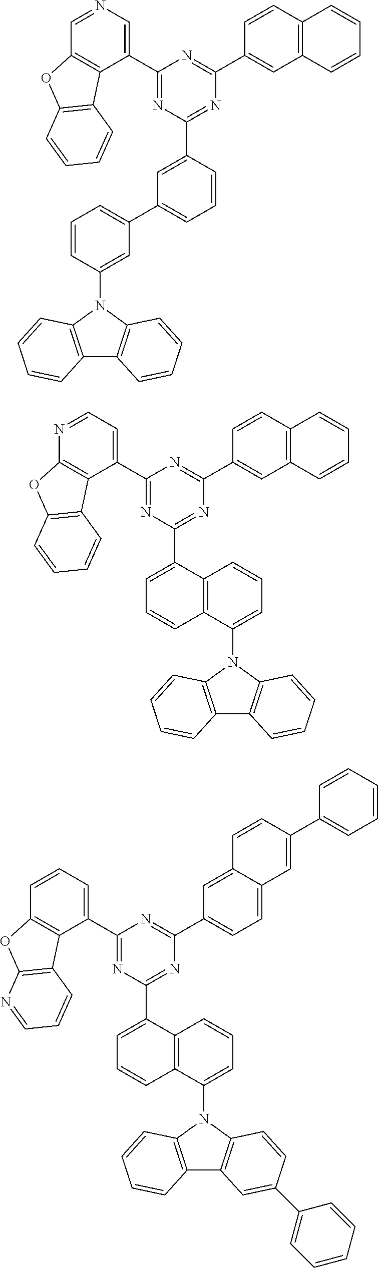

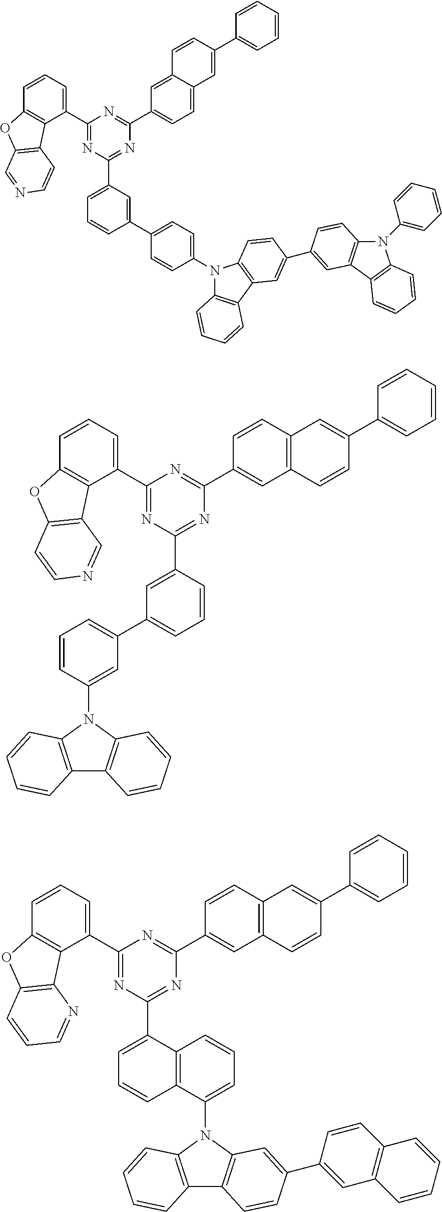 [SMILES: c1ccc(-c2ccc3cc(-c4nc(-c5cccc(-c6ccc(-n7c8ccccc8c8cc(-c9ccc%10c(c9)c9ccccc9n%10-c9ccccc9)ccc87)cc6)c5)nc(-c5cccc6oc7cnccc7c56)n4)ccc3c2)cc1 ; c1ccc(-c2ccc3cc(-c4nc(-c5cccc(-c6cccc(-n7c8ccccc8c8ccccc87)c6)c5)nc(-c5cccc6oc7ccncc7c56)n4)ccc3c2)cc1 ; c1ccc(-c2ccc3cc(-c4nc(-c5cccc6c(-n7c8ccccc8c8ccc(-c9ccc%10ccccc%10c9)cc87)cccc56)nc(-c5cccc6oc7cccnc7c56)n4)ccc3c2)cc1]

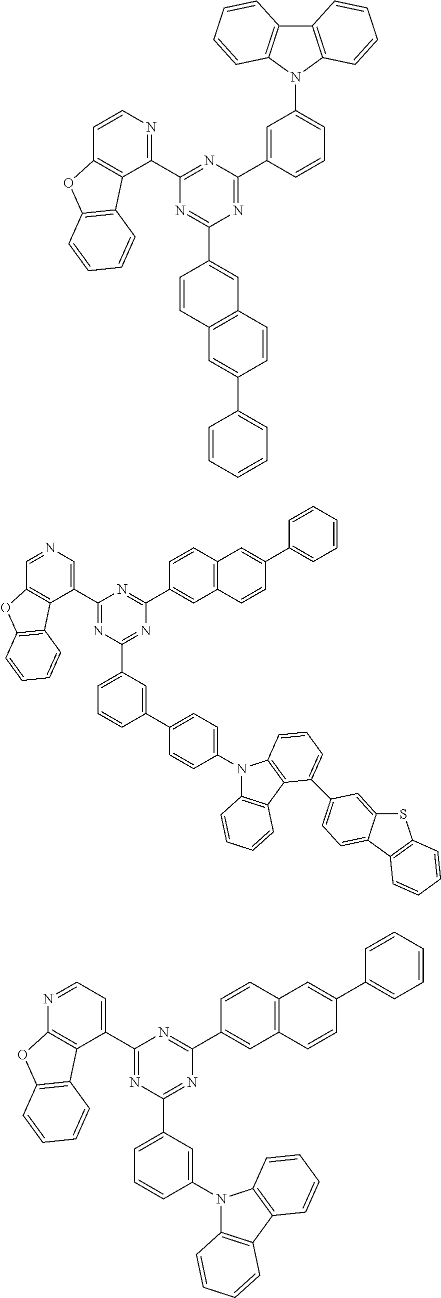

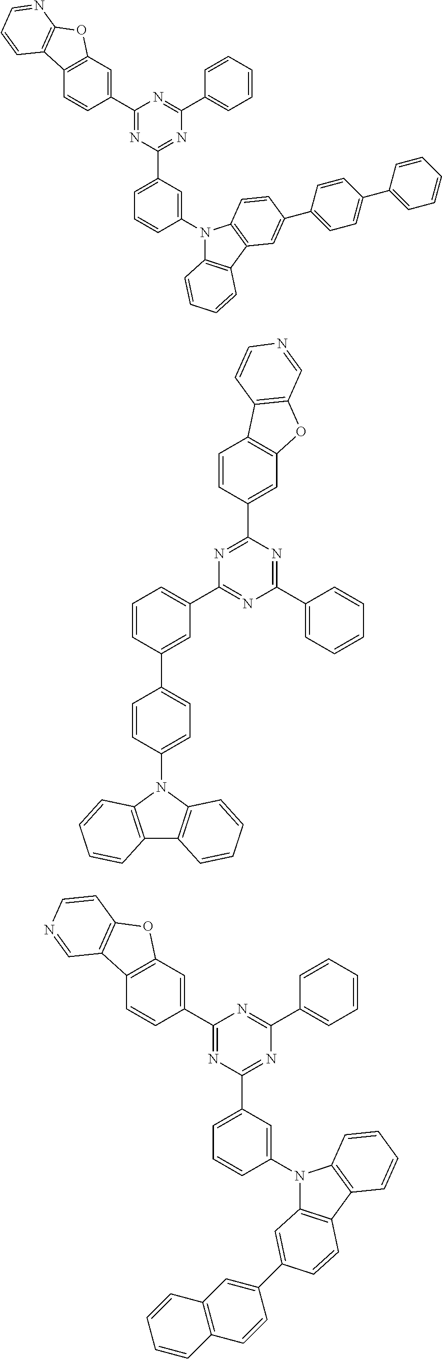

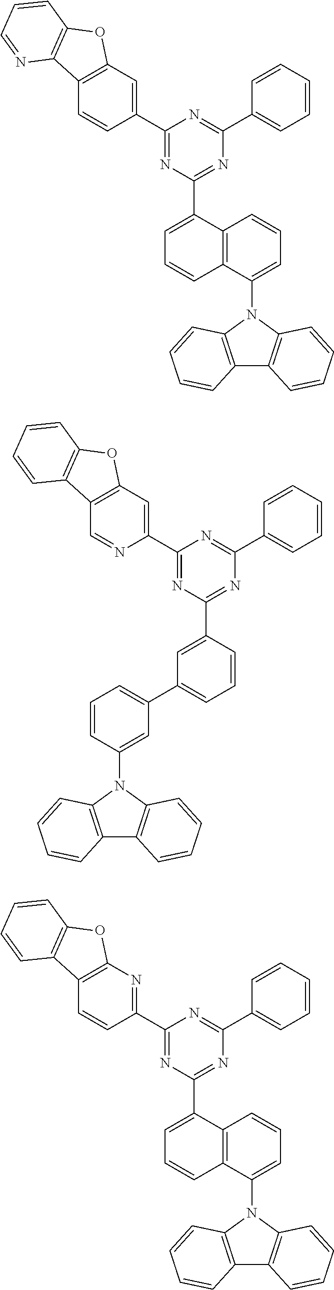

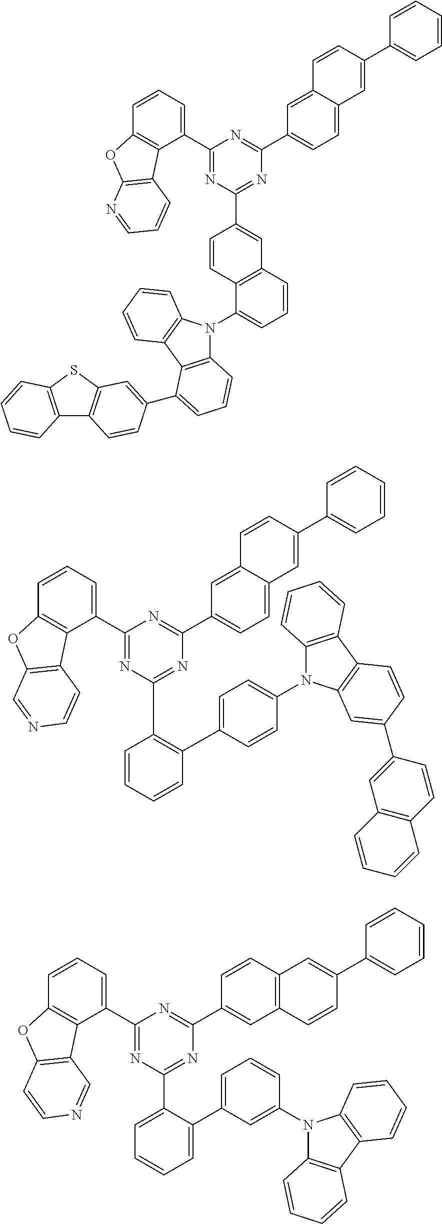

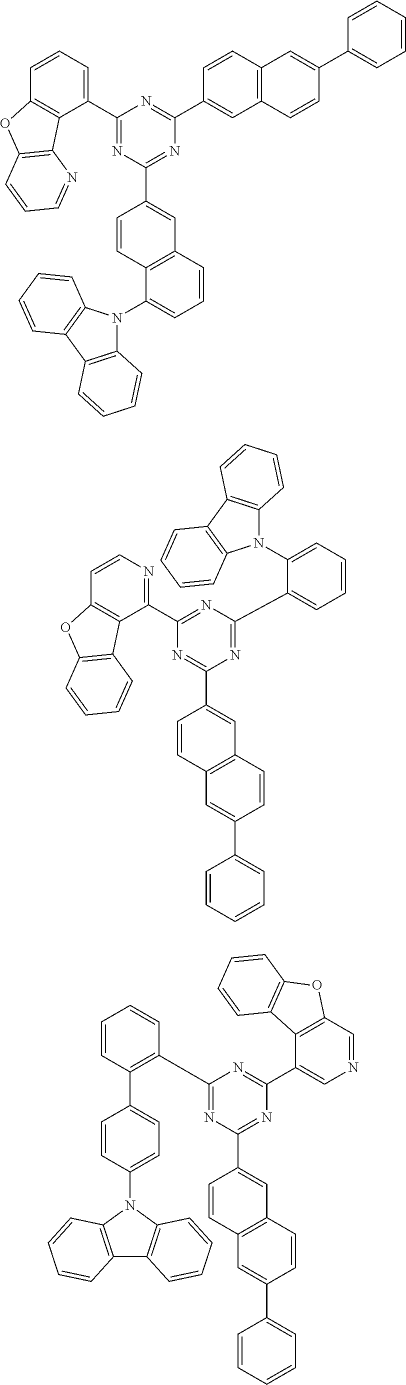

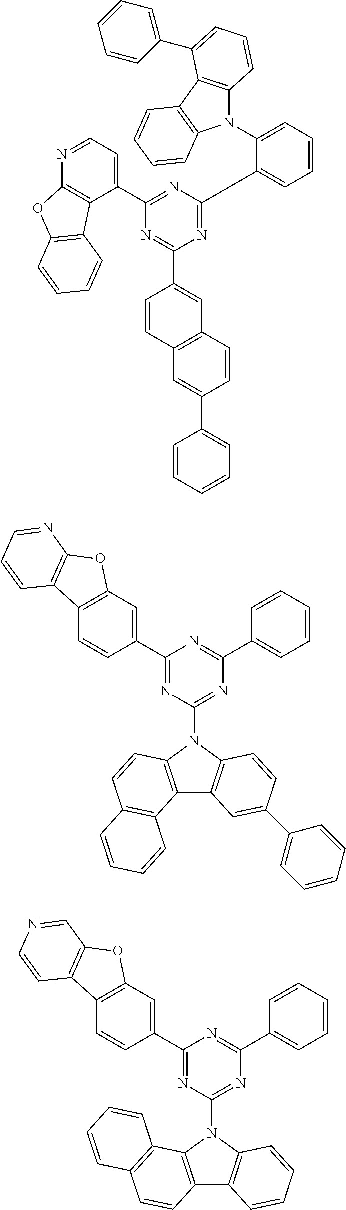 [SMILES: c1ccc(-c2ccc3cc(-c4nc(-c5ccccc5-n5c6ccccc6c6c(-c7ccccc7)cccc65)nc(-c5ccnc6oc7ccccc7c56)n4)ccc3c2)cc1 ; c1ccc(-c2ccc3c(c2)c2c4ccccc4ccc2n3-c2nc(-c3ccccc3)nc(-c3ccc4c(c3)oc3ncccc34)n2)cc1 ; c1ccc(-c2nc(-c3ccc4c(c3)oc3cnccc34)nc(-n3c4ccccc4c4ccc5ccccc5c43)n2)cc1]

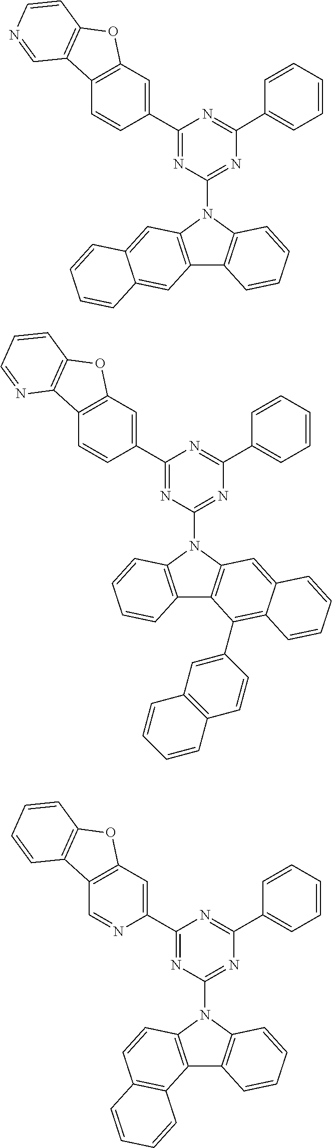

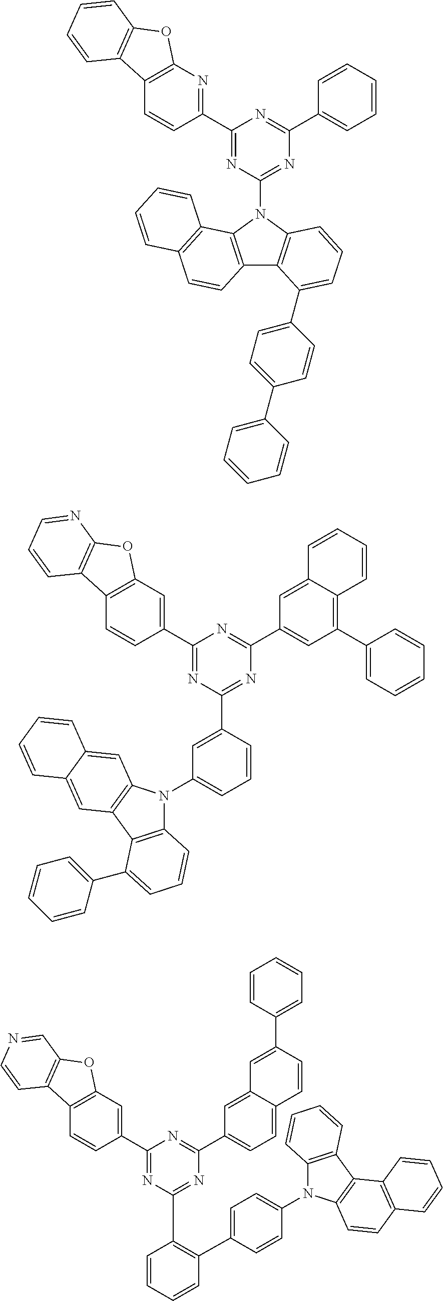 [SMILES: c1ccc(-c2ccc(-c3cccc4c3c3ccc5ccccc5c3n4-c3nc(-c4ccccc4)nc(-c4ccc5c(n4)oc4ccccc45)n3)cc2)cc1 ; c1ccc(-c2cc(-c3nc(-c4cccc(-n5c6cc7ccccc7cc6c6c(-c7ccccc7)cccc65)c4)nc(-c4ccc5c(c4)oc4ncccc45)n3)cc3ccccc23)cc1 ; c1ccc(-c2ccc3ccc(-c4nc(-c5ccc6c(c5)oc5cnccc56)nc(-c5ccccc5-c5ccc(-n6c7ccccc7c7c8ccccc8ccc76)cc5)n4)cc3c2)cc1]

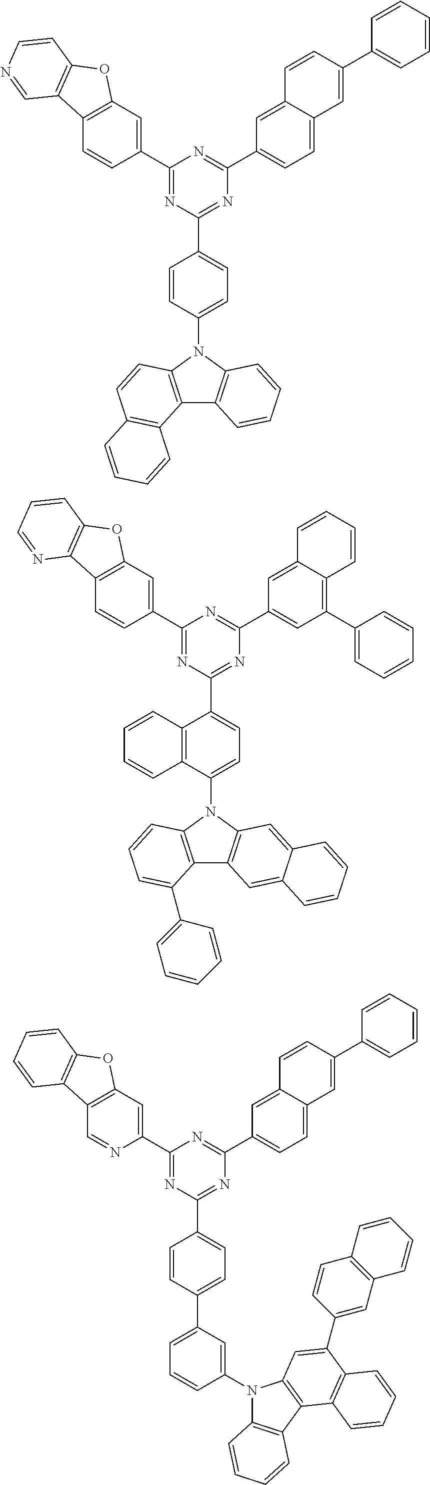 [SMILES: c1ccc(-c2ccc3cc(-c4nc(-c5ccc(-n6c7ccccc7c7c8ccccc8ccc76)cc5)nc(-c5ccc6c(c5)oc5ccncc56)n4)ccc3c2)cc1 ; c1ccc(-c2cc(-c3nc(-c4ccc5c(c4)oc4cccnc45)nc(-c4ccc(-n5c6cc7ccccc7cc6c6c(-c7ccccc7)cccc65)c5ccccc45)n3)cc3ccccc23)cc1 ; c1ccc(-c2ccc3cc(-c4nc(-c5ccc(-c6cccc(-n7c8ccccc8c8c9ccccc9c(-c9ccc%10ccccc%10c9)cc87)c6)cc5)nc(-c5cc6oc7ccccc7c6cn5)n4)ccc3c2)cc1]

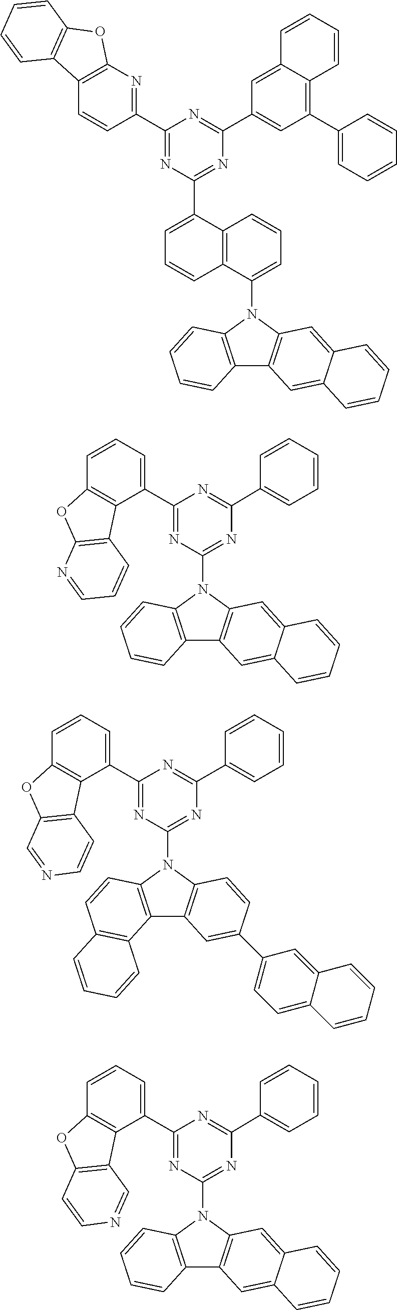 [SMILES: c1ccc(-c2cc(-c3nc(-c4ccc5c(n4)oc4ccccc45)nc(-c4cccc5c(-n6c7ccccc7c7cc8ccccc8cc76)cccc45)n3)cc3ccccc23)cc1 ; c1ccc(-c2nc(-c3cccc4oc5ncccc5c34)nc(-n3c4ccccc4c4cc5ccccc5cc43)n2)cc1 ; c1ccc(-c2nc(-c3cccc4oc5cnccc5c34)nc(-n3c4ccc(-c5ccc6ccccc6c5)cc4c4c5ccccc5ccc43)n2)cc1 ; c1ccc(-c2nc(-c3cccc4oc5ccncc5c34)nc(-n3c4ccccc4c4cc5ccccc5cc43)n2)cc1]

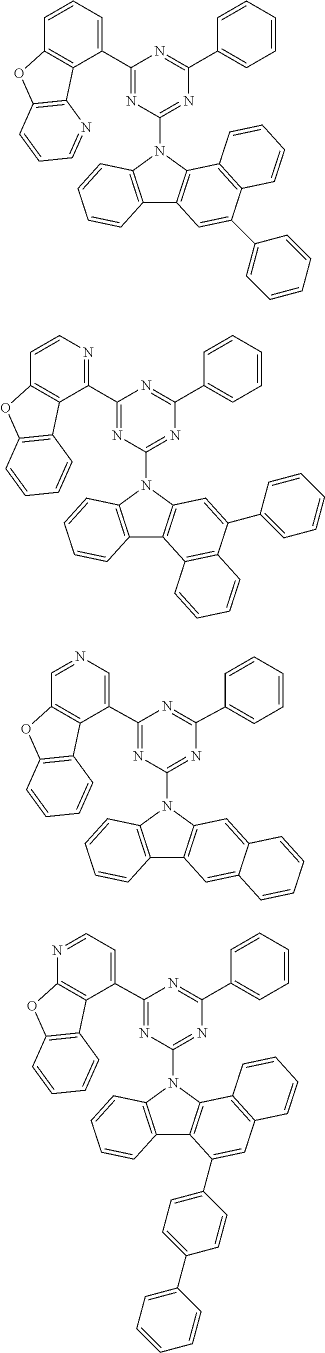

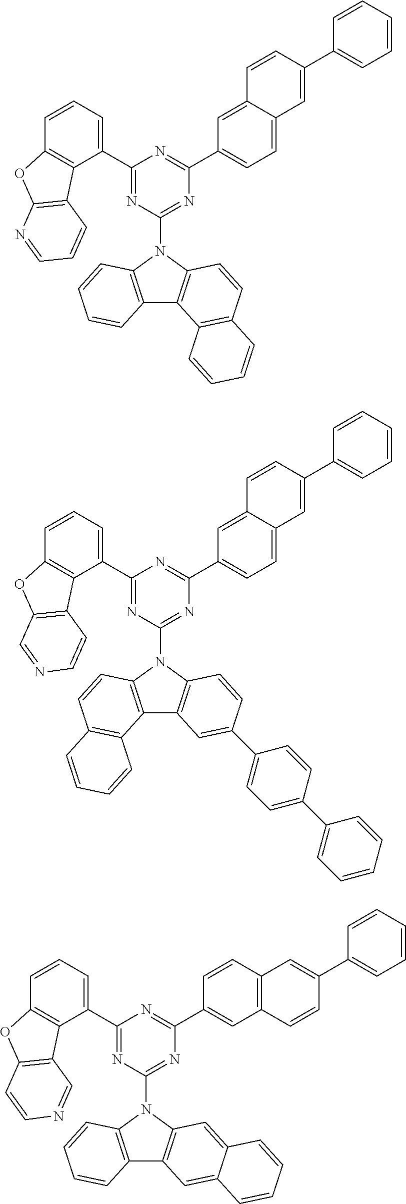

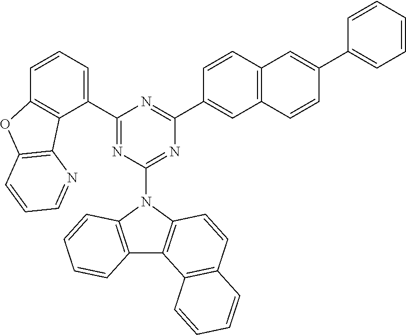 [SMILES: c1ccc(-c2ccc3cc(-c4nc(-c5cccc6oc7cccnc7c56)nc(-n5c6ccccc6c6c7ccccc7ccc65)n4)ccc3c2)cc1]

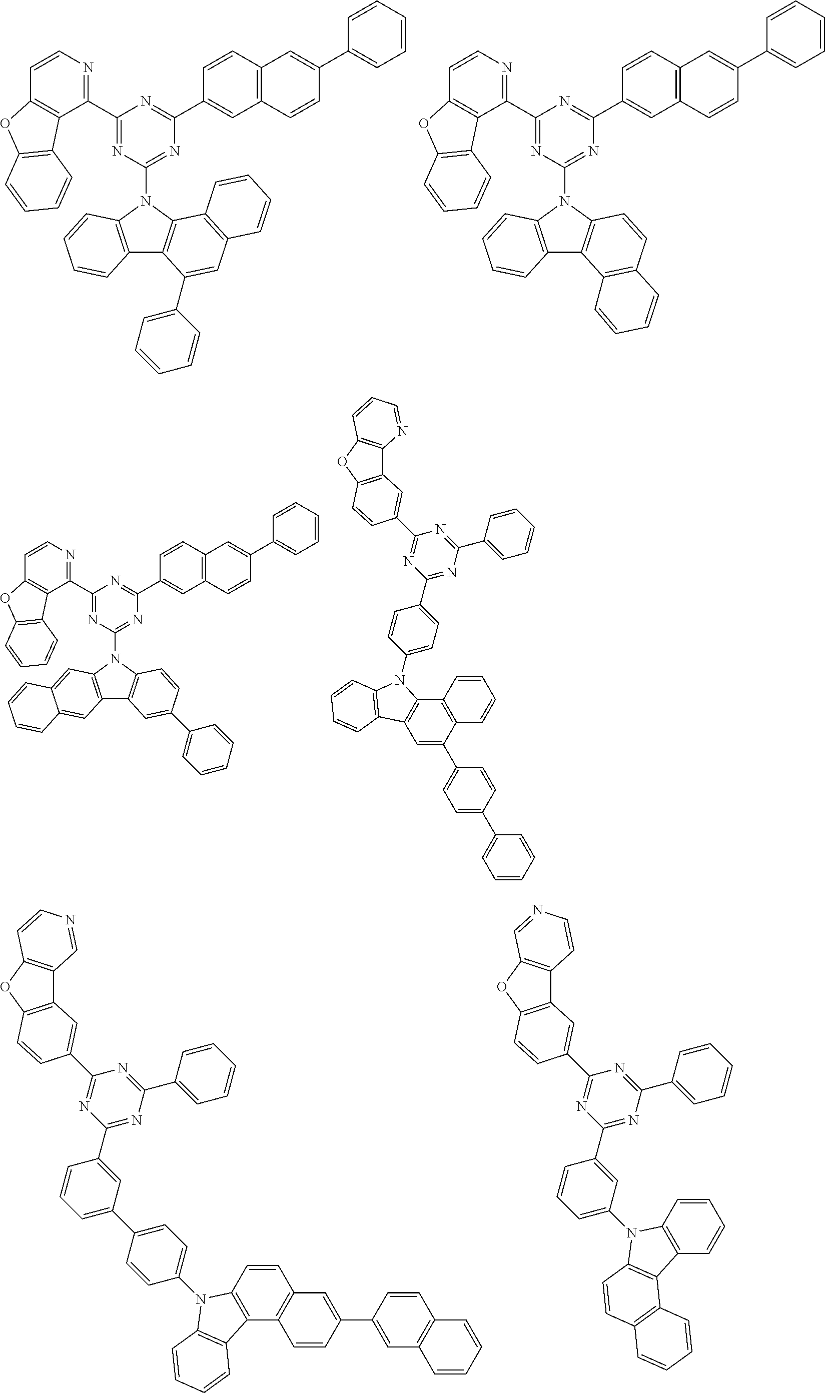

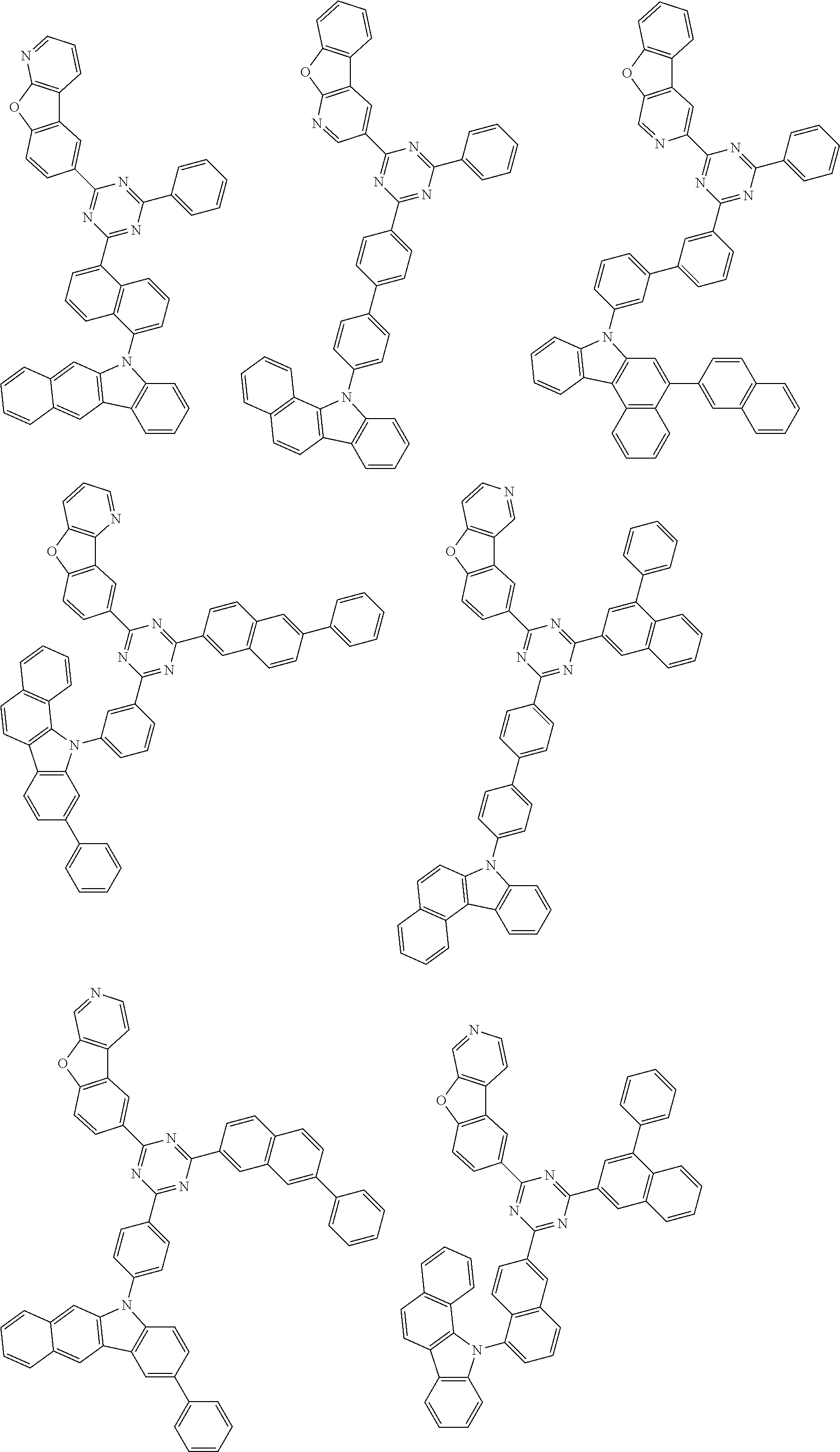

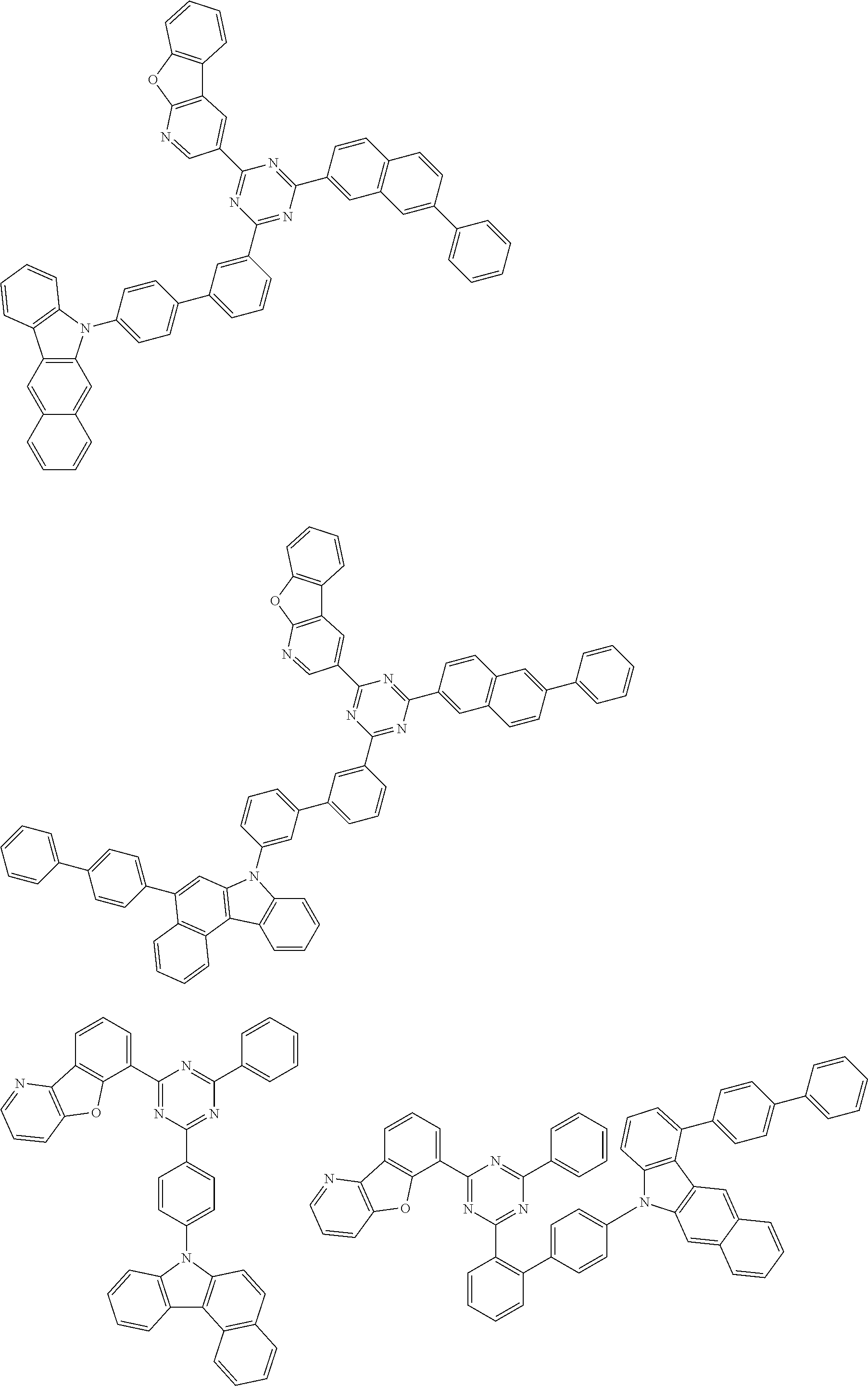 [SMILES: c1ccc(-c2ccc3ccc(-c4nc(-c5cccc(-c6ccc(-n7c8ccccc8c8cc9ccccc9cc87)cc6)c5)nc(-c5cnc6oc7ccccc7c6c5)n4)cc3c2)cc1 ; c1ccc(-c2ccc(-c3cc4c(c5ccccc35)c3ccccc3n4-c3cccc(-c4cccc(-c5nc(-c6ccc7cc(-c8ccccc8)ccc7c6)nc(-c6cnc7oc8ccccc8c7c6)n5)c4)c3)cc2)cc1 ; c1ccc(-c2nc(-c3ccc(-n4c5ccccc5c5c6ccccc6ccc54)cc3)nc(-c3cccc4c3oc3cccnc34)n2)cc1 ; c1ccc(-c2ccc(-c3cccc4c3c3cc5ccccc5cc3n4-c3ccc(-c4ccccc4-c4nc(-c5ccccc5)nc(-c5cccc6c5oc5cccnc56)n4)cc3)cc2)cc1]

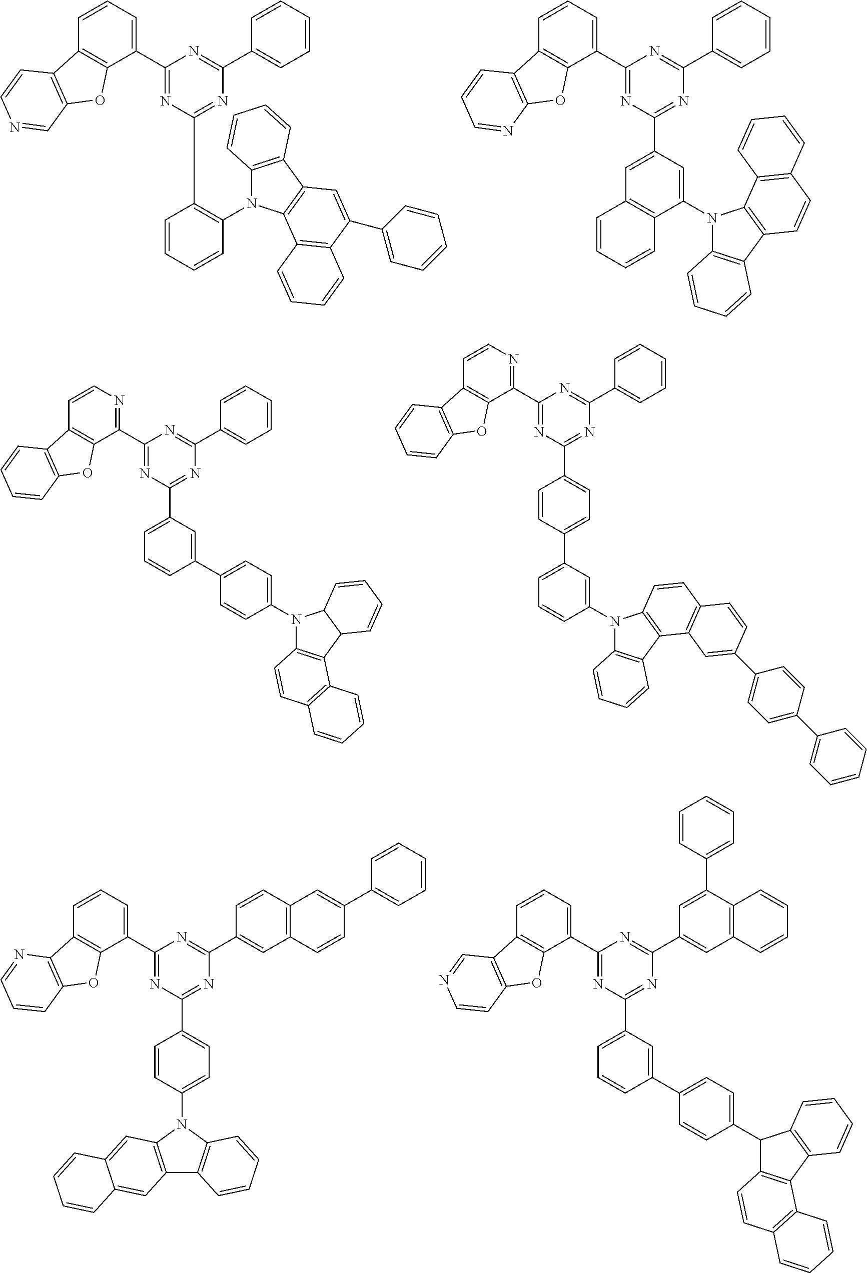

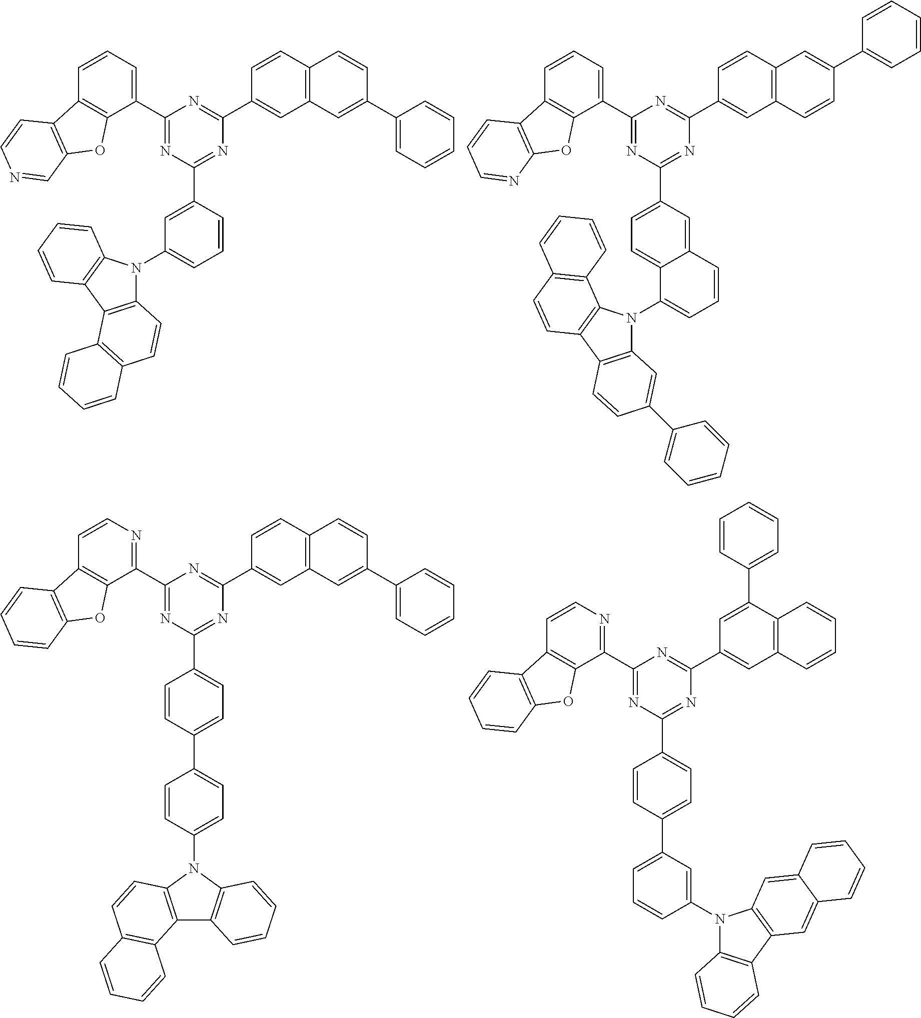

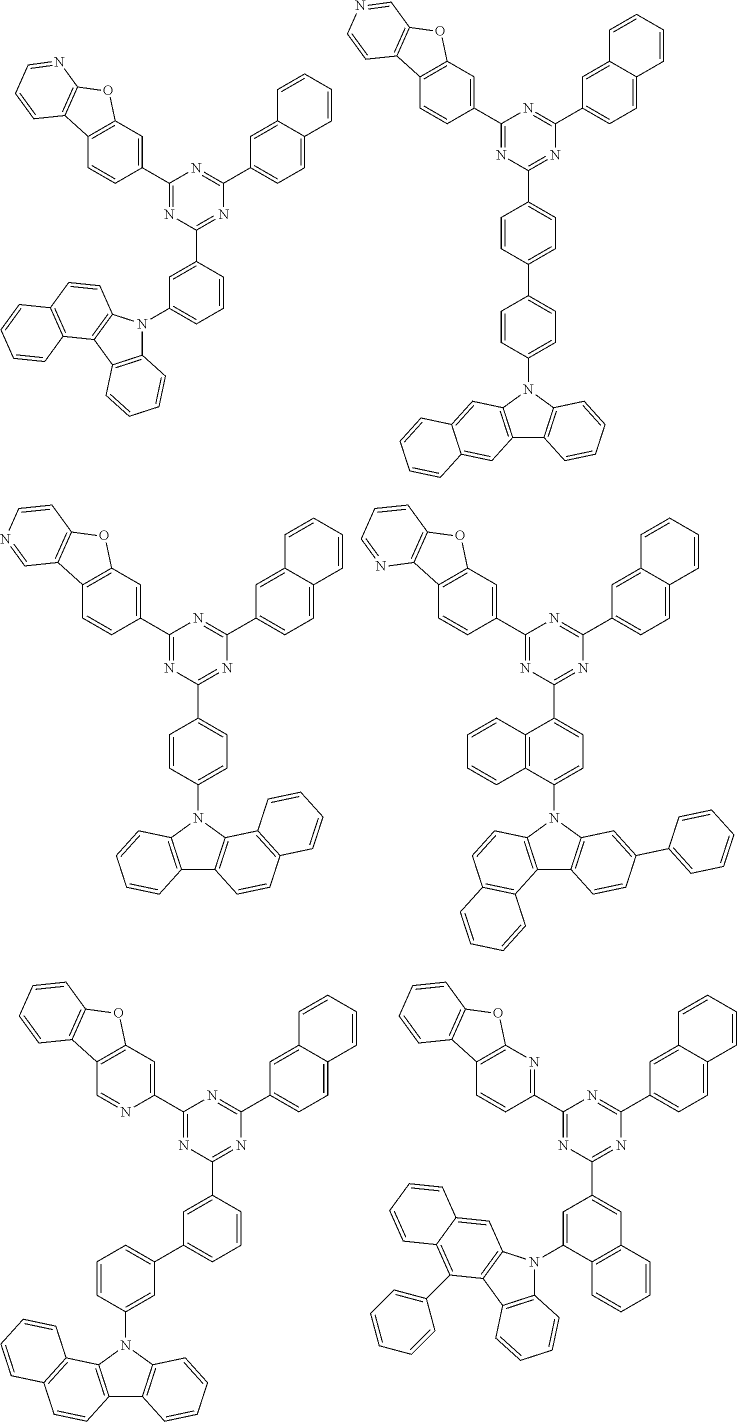

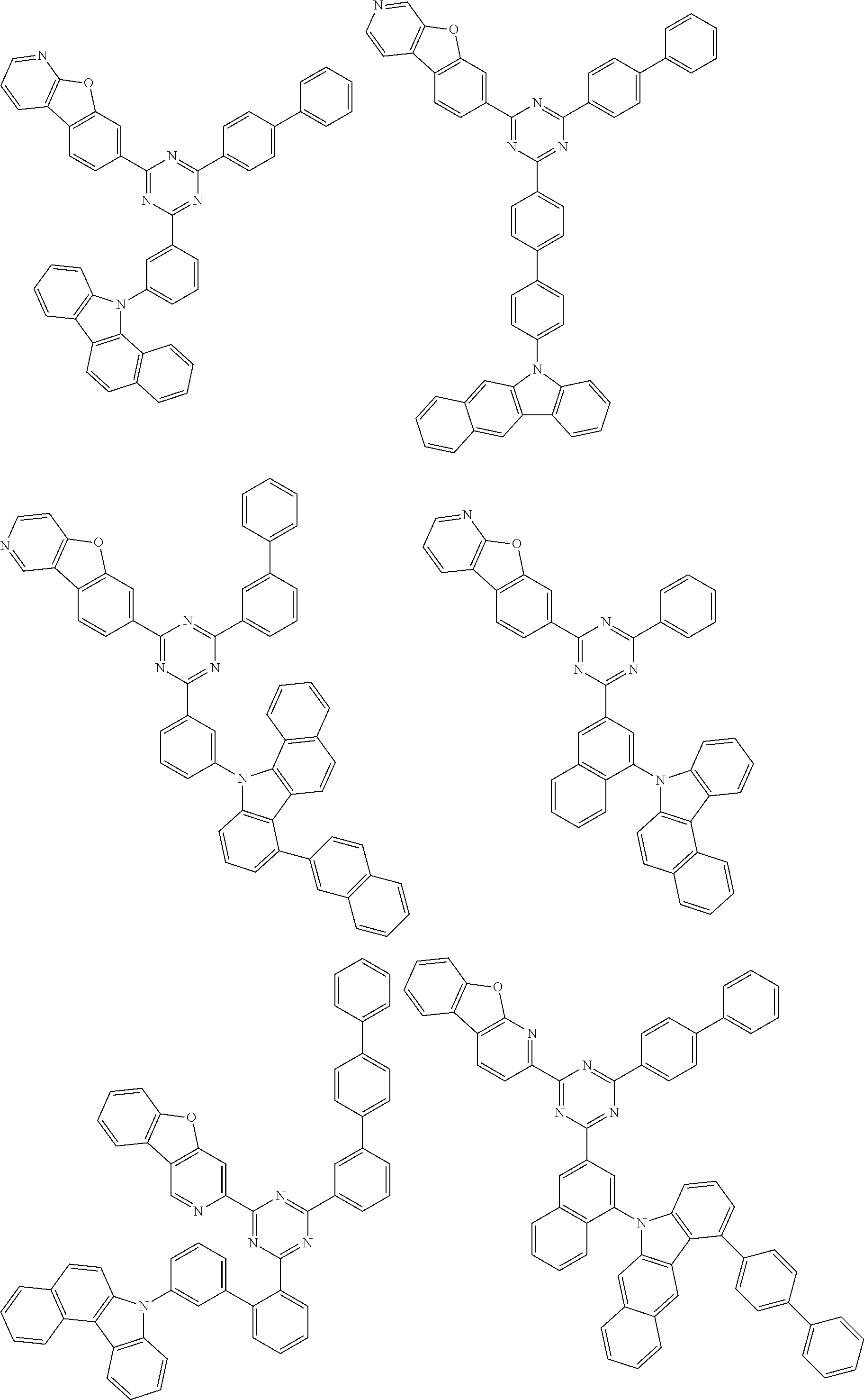

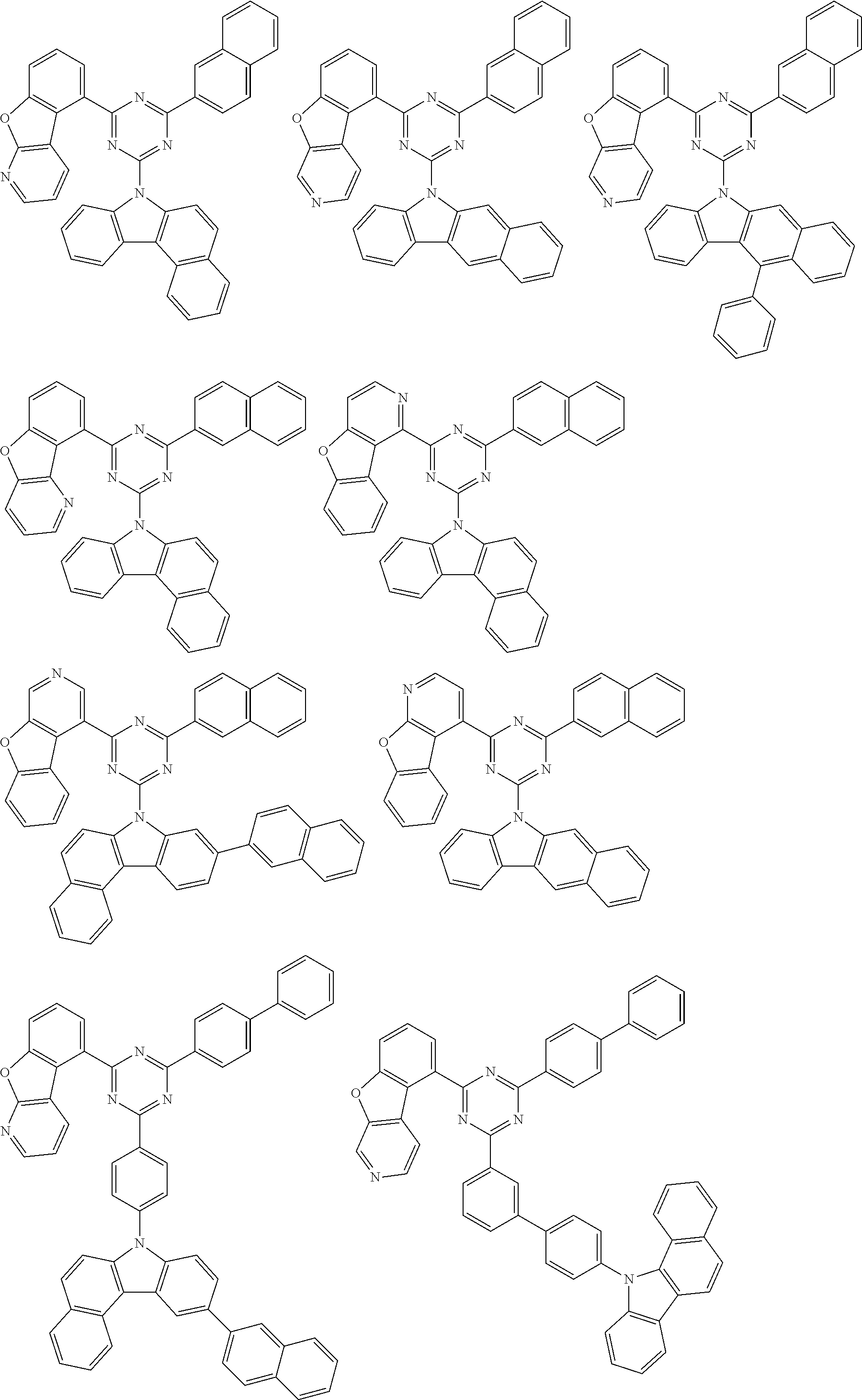

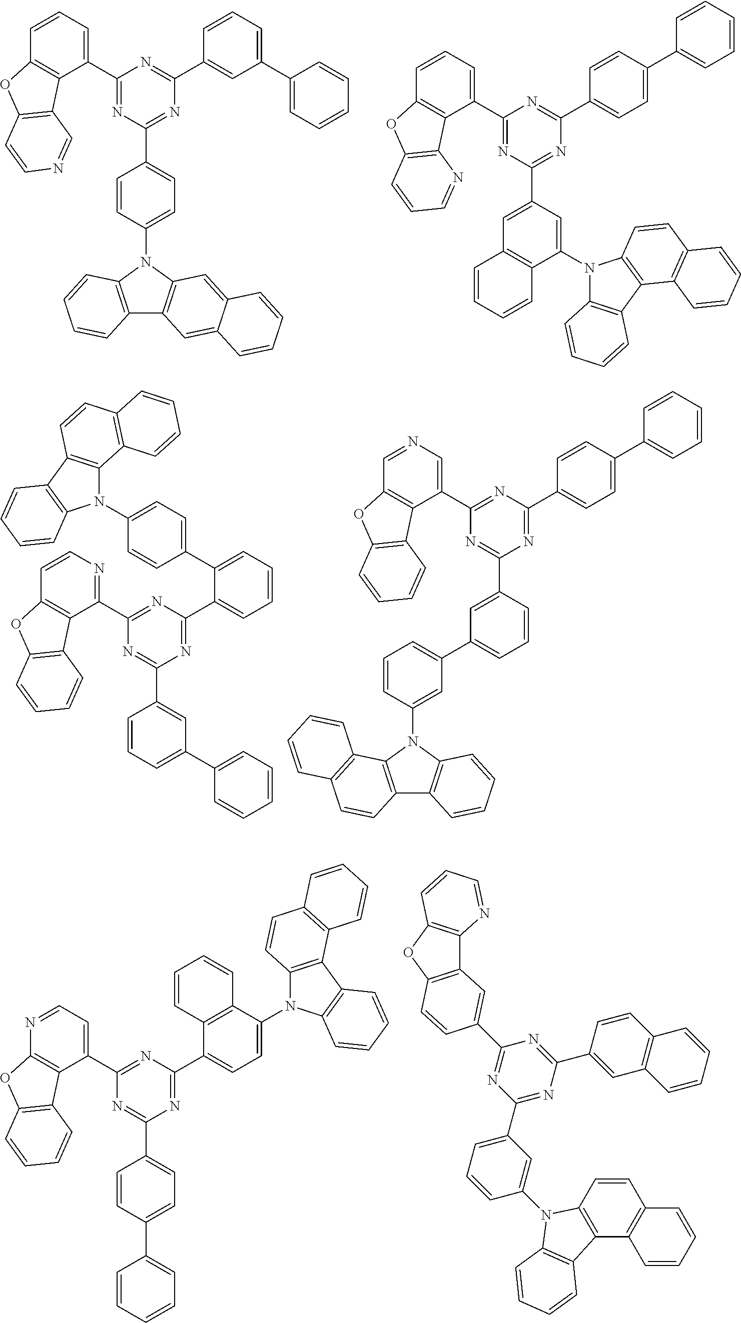

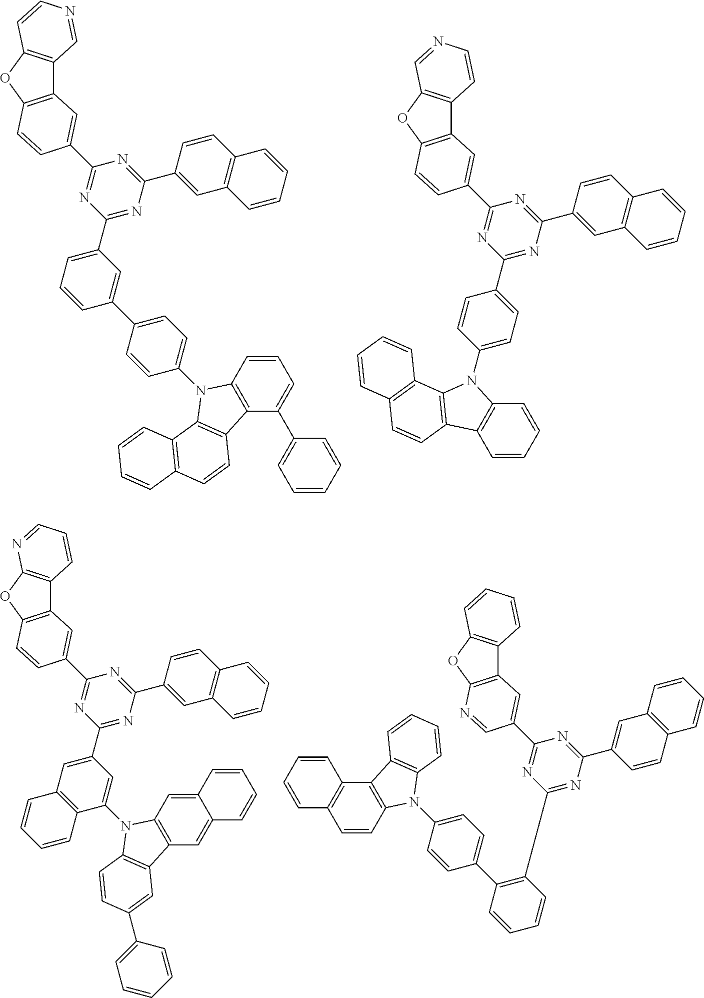

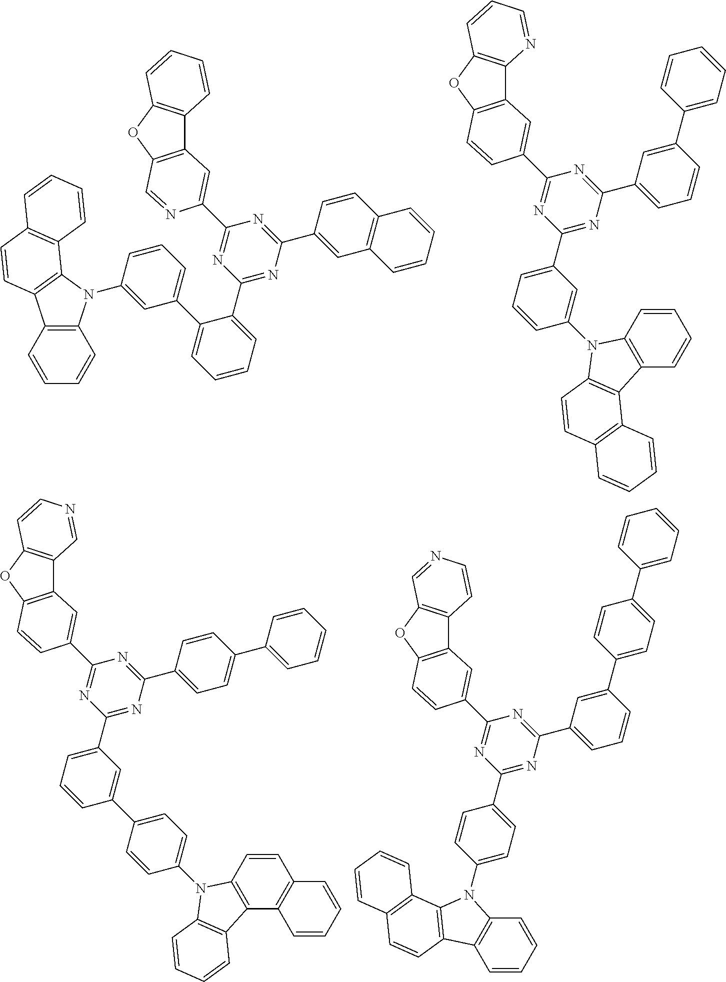

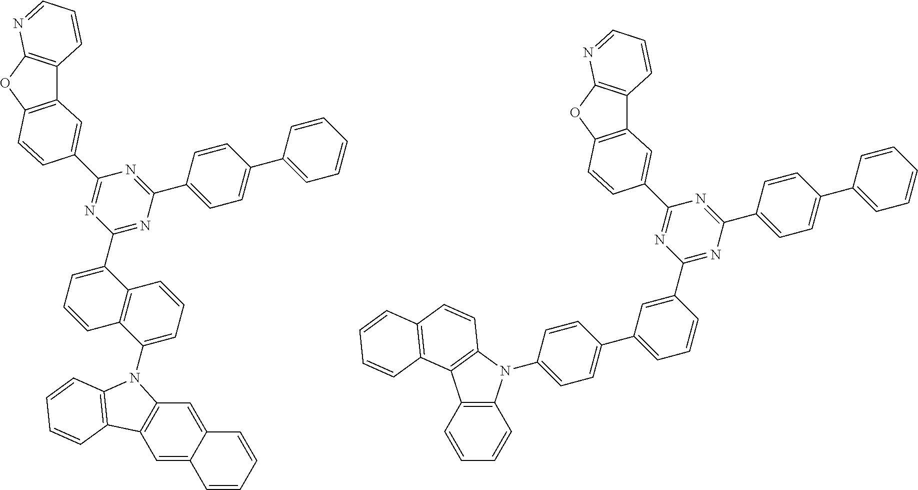 [SMILES: c1ccc(-c2ccc(-c3nc(-c4ccc5oc6ncccc6c5c4)nc(-c4cccc5c(-n6c7ccccc7c7cc8ccccc8cc76)cccc45)n3)cc2)cc1 ; c1ccc(-c2ccc(-c3nc(-c4cccc(-c5ccc(-n6c7ccccc7c7c8ccccc8ccc76)cc5)c4)nc(-c4ccc5oc6ncccc6c5c4)n3)cc2)cc1]

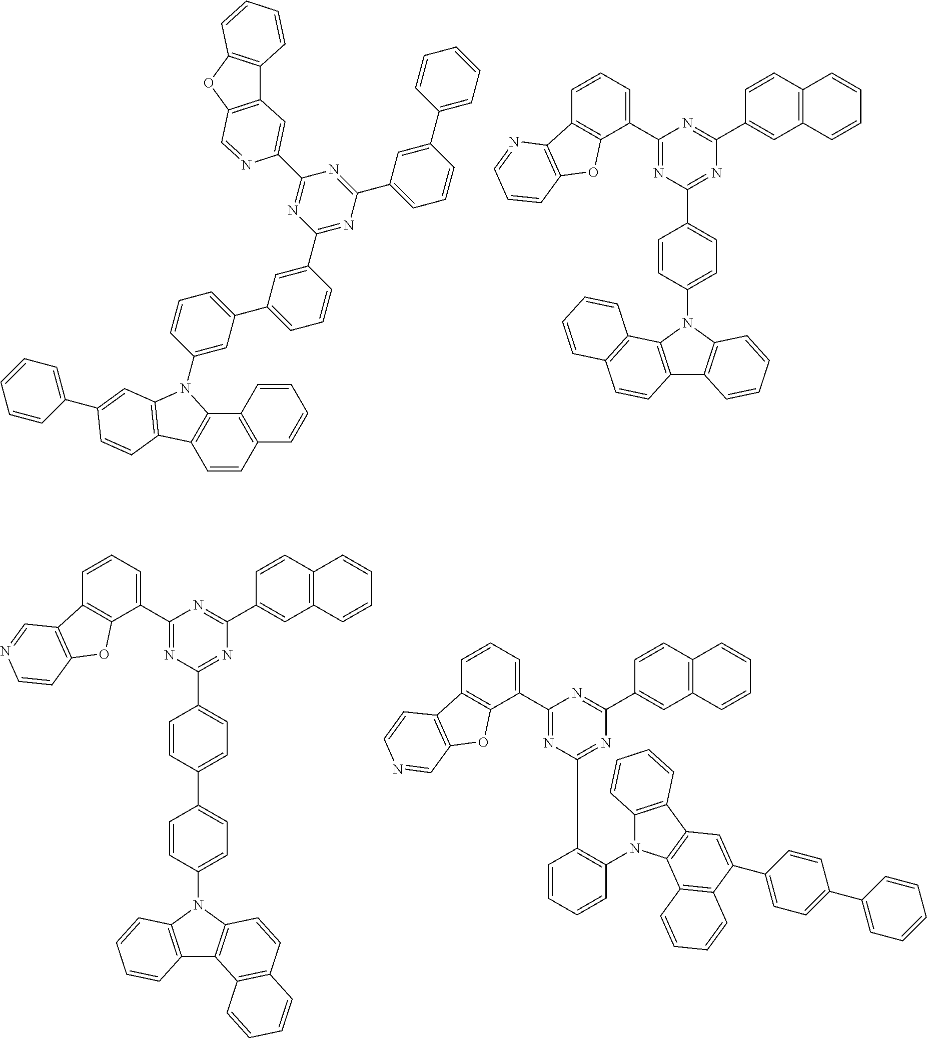

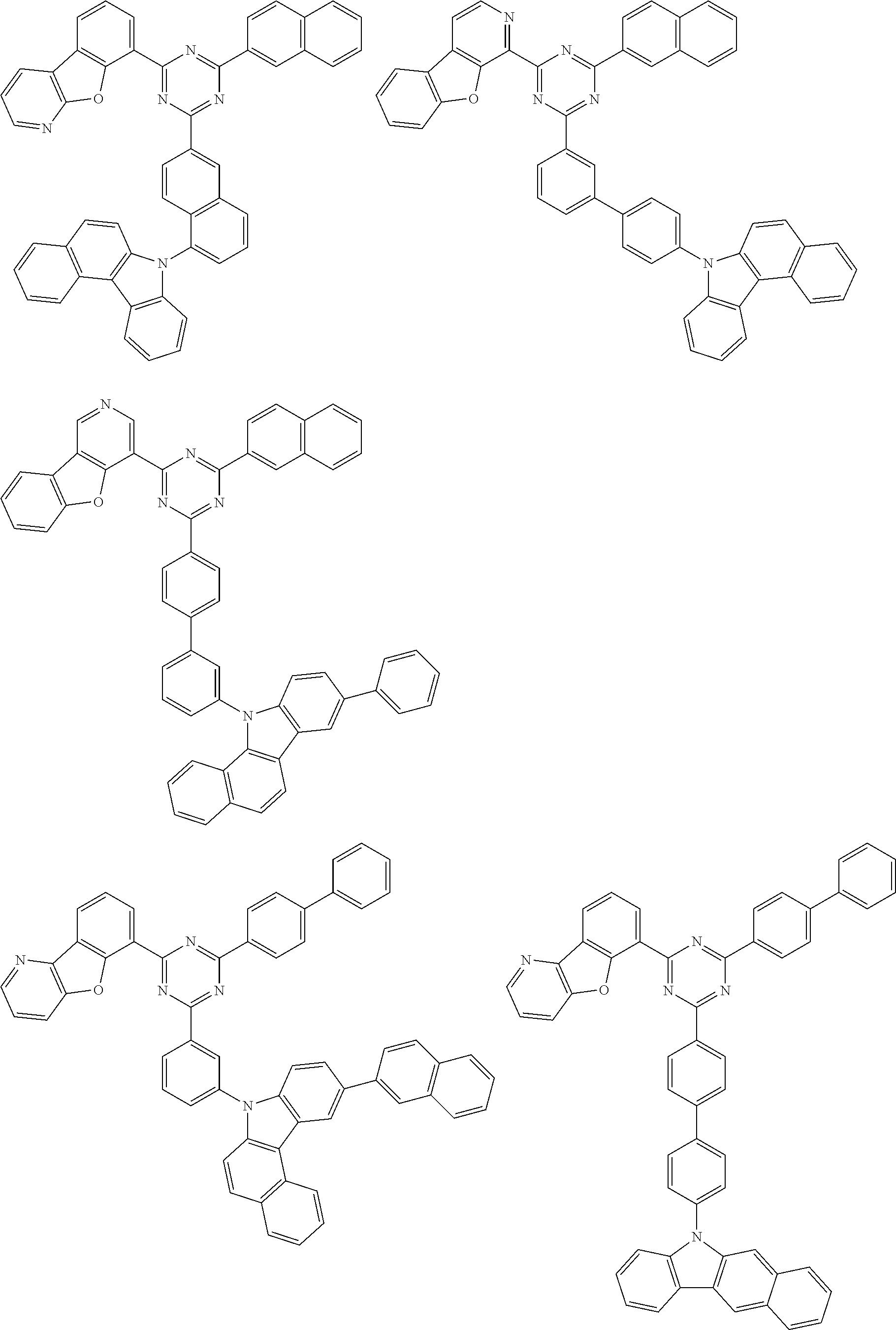

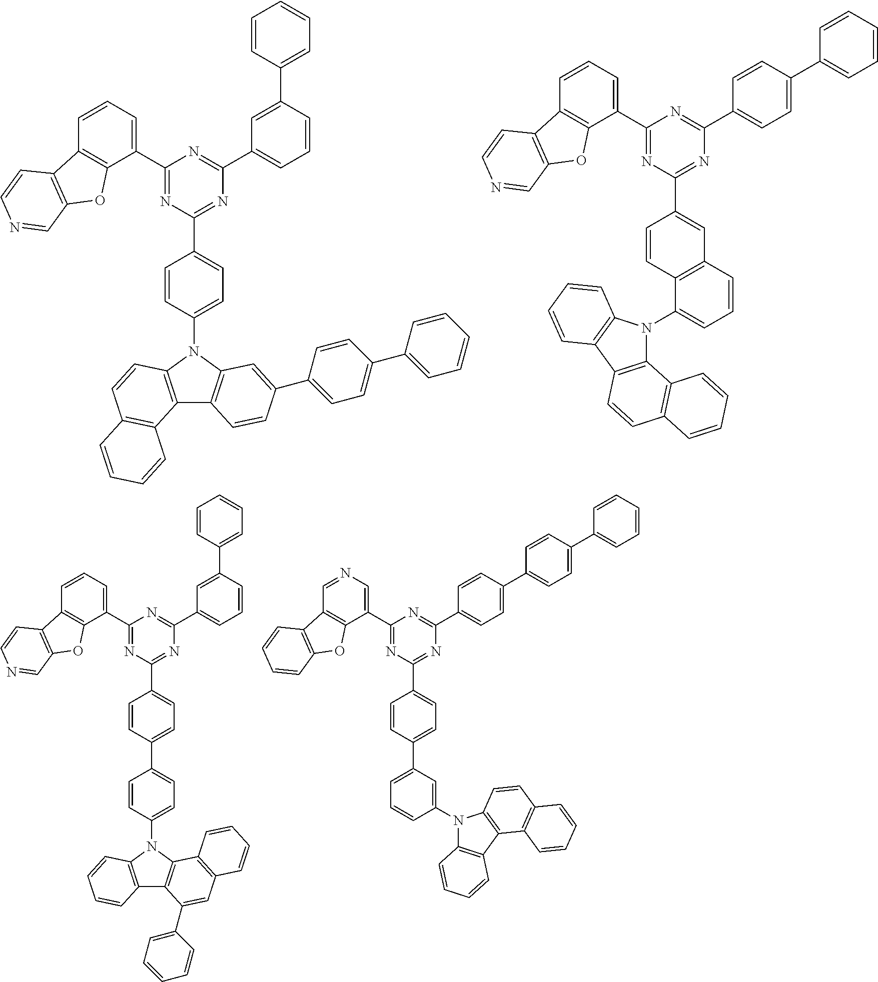 [SMILES: c1ccc(-c2ccc(-c3ccc4c5c6ccccc6ccc5n(-c5ccc(-c6nc(-c7cccc(-c8ccccc8)c7)nc(-c7cccc8c7oc7cnccc78)n6)cc5)c4c3)cc2)cc1 ; c1ccc(-c2ccc(-c3nc(-c4ccc5c(-n6c7ccccc7c7ccc8ccccc8c76)cccc5c4)nc(-c4cccc5c4oc4cnccc45)n3)cc2)cc1 ; c1ccc(-c2cccc(-c3nc(-c4ccc(-c5ccc(-n6c7ccccc7c7c(-c8ccccc8)cc8ccccc8c76)cc5)cc4)nc(-c4cccc5c4oc4cnccc45)n3)c2)cc1 ; c1ccc(-c2ccc(-c3ccc(-c4nc(-c5ccc(-c6cccc(-n7c8ccccc8c8c9ccccc9ccc87)c6)cc5)nc(-c5cncc6c5oc5ccccc56)n4)cc3)cc2)cc1]

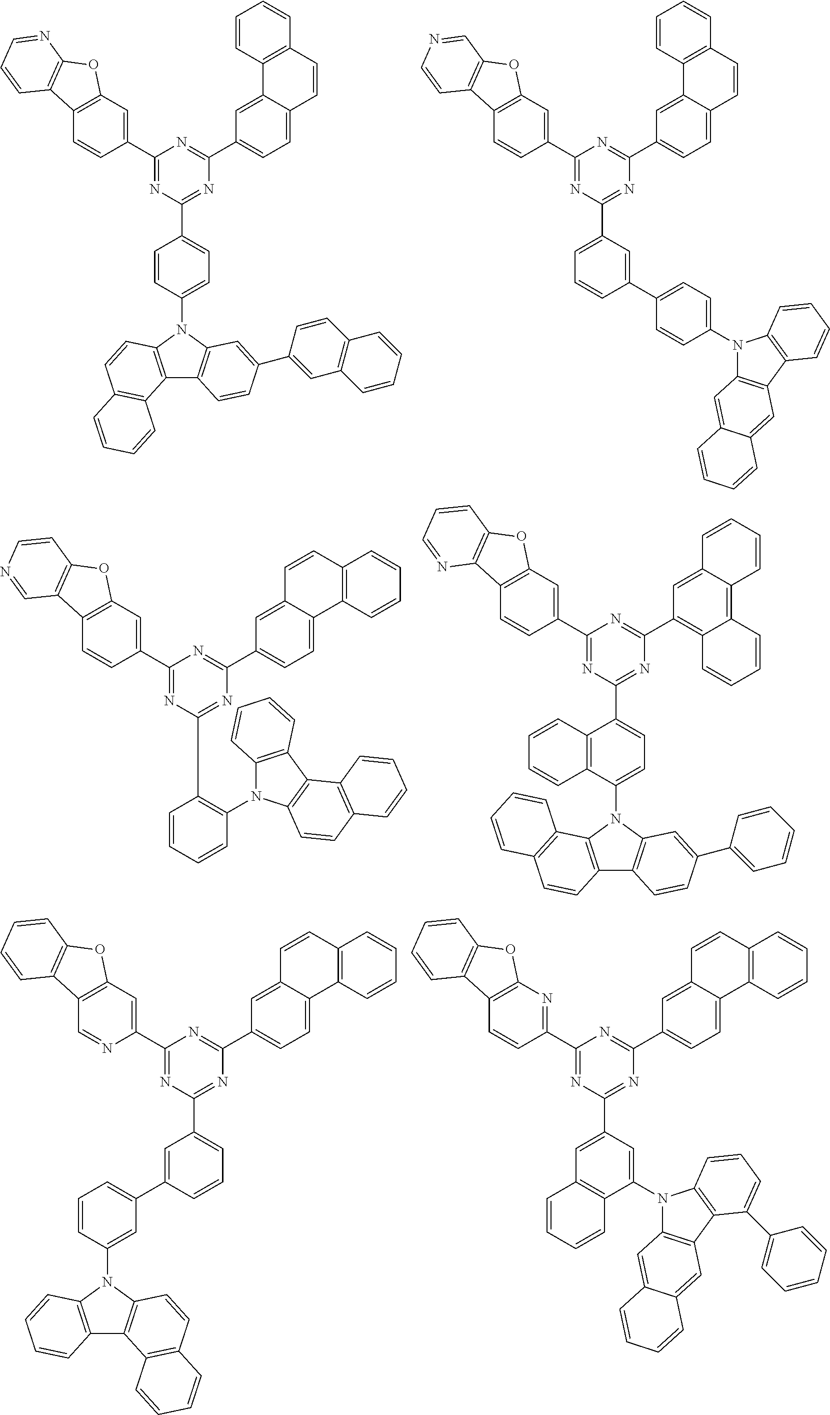

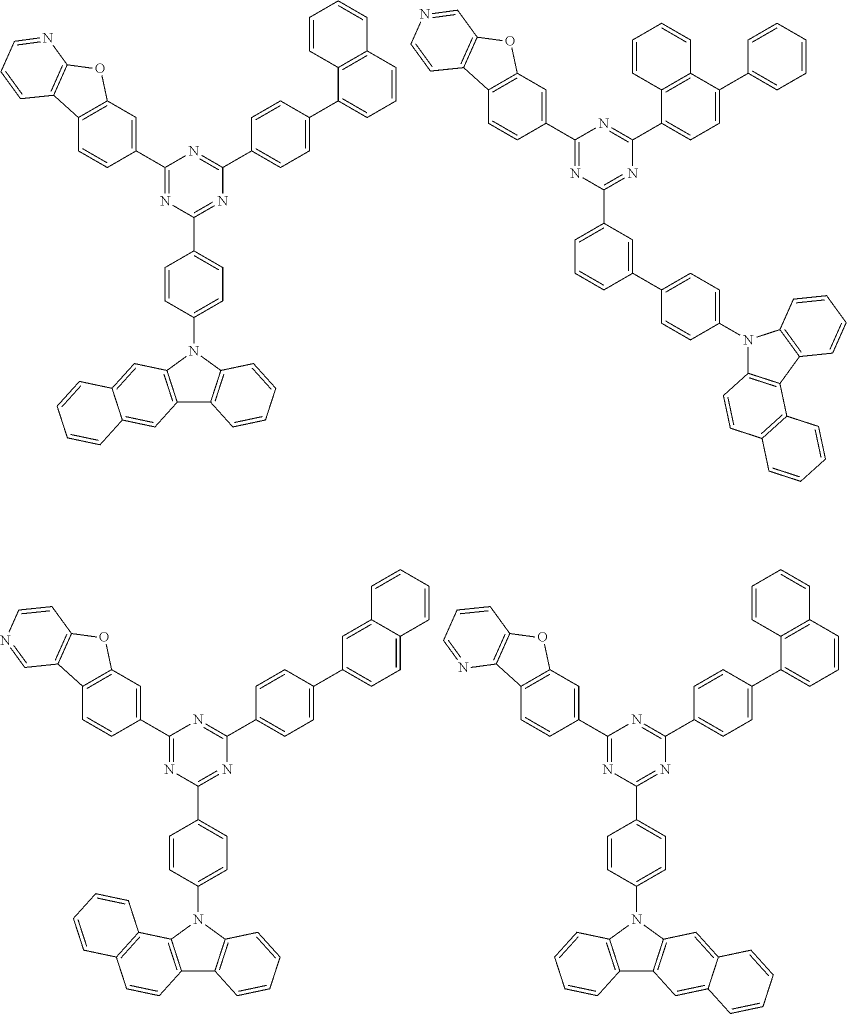

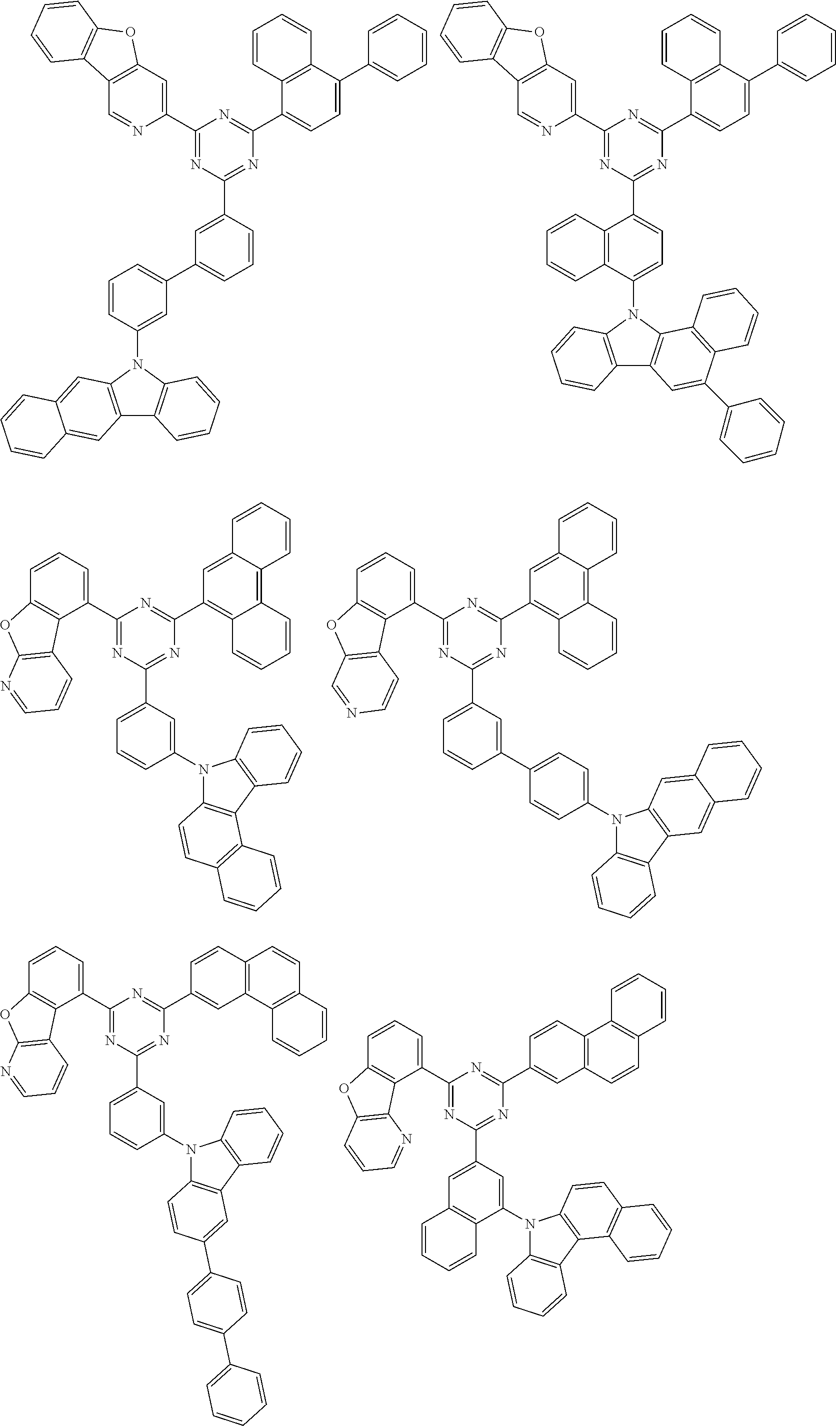

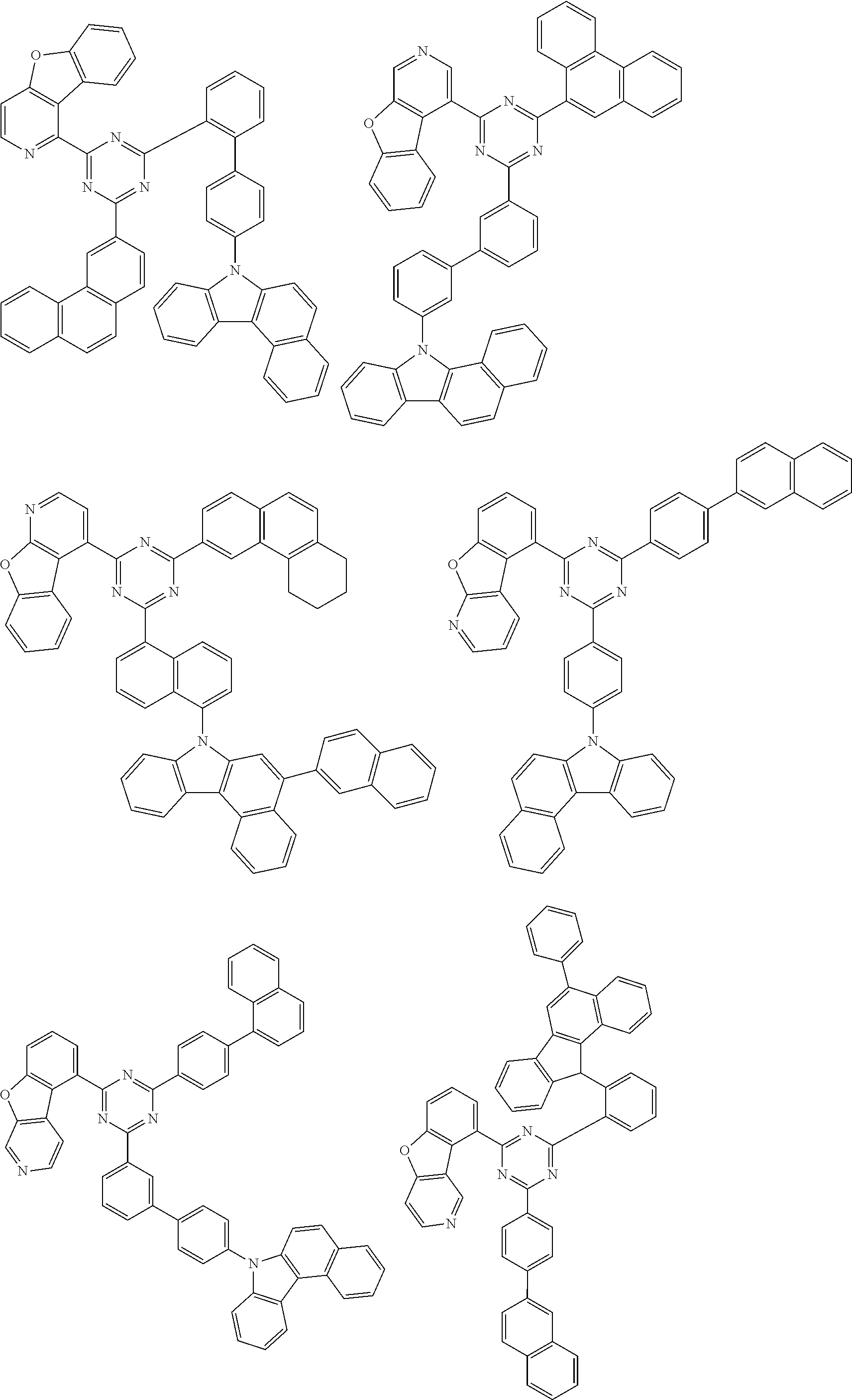

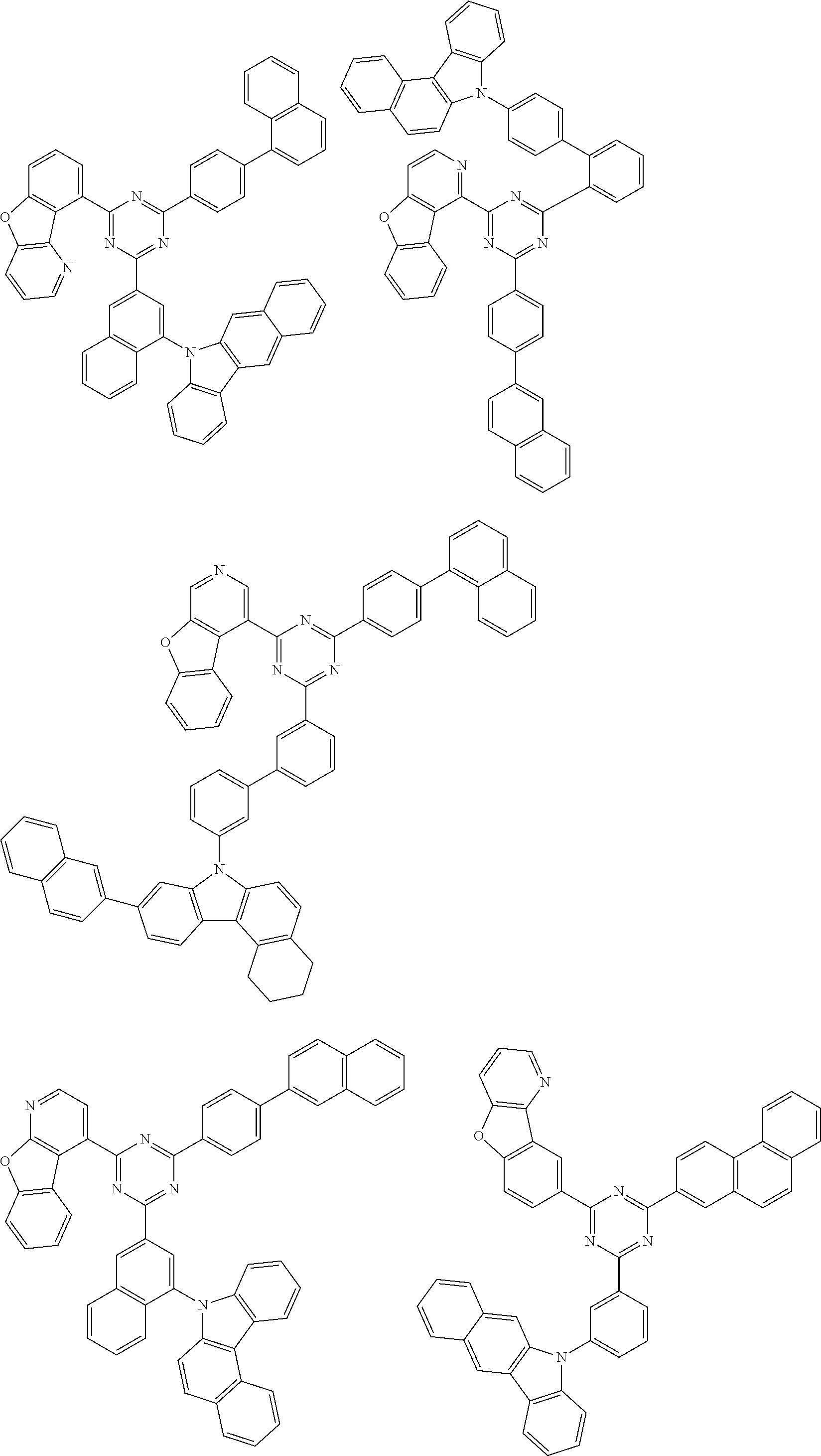

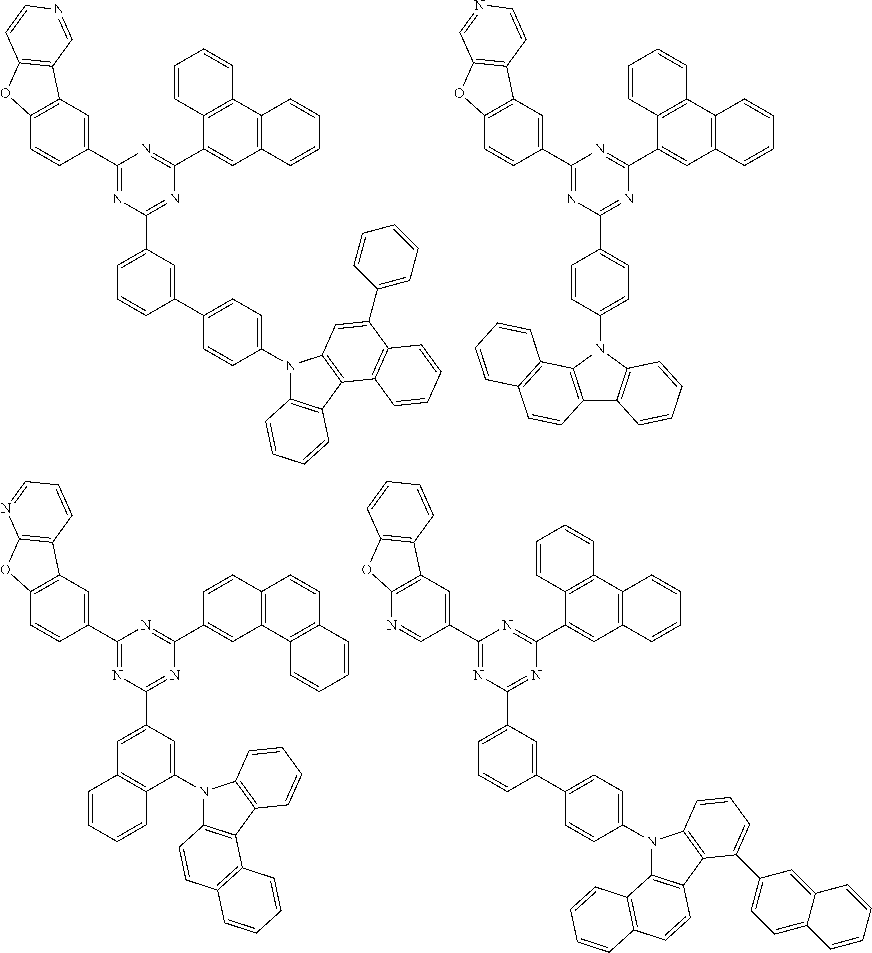

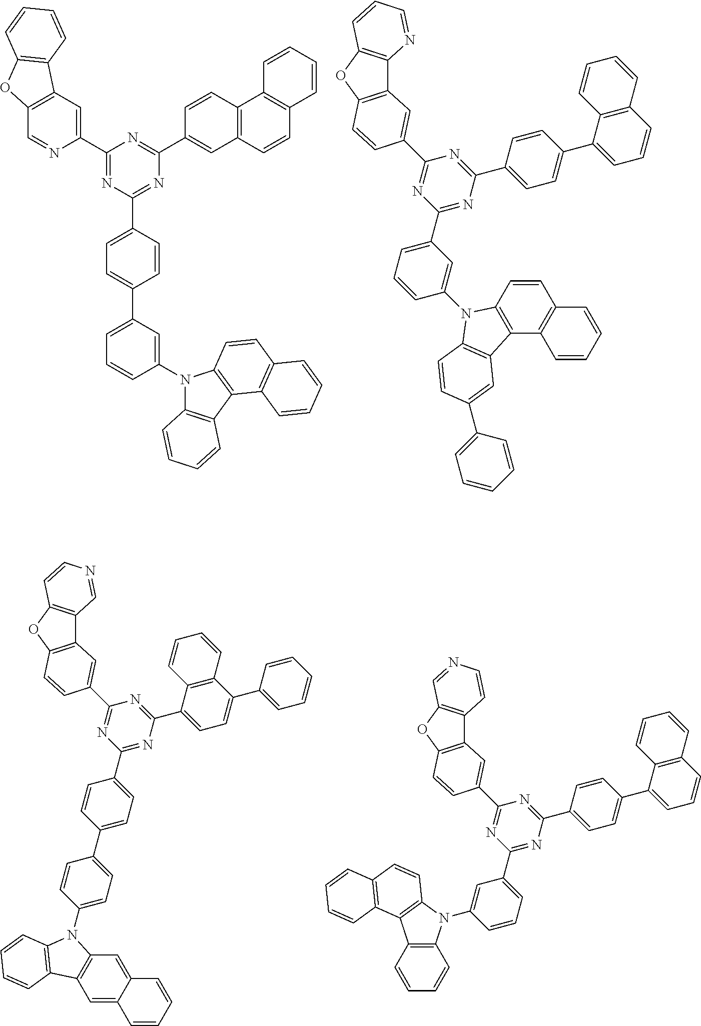

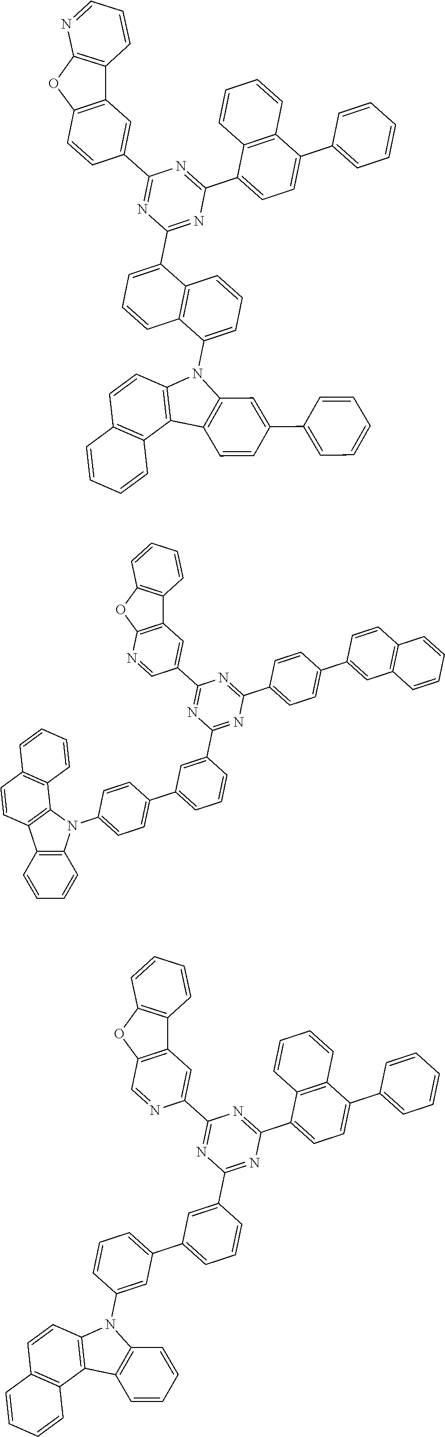 [SMILES: c1ccc(-c2ccc3c4c5ccccc5ccc4n(-c4cccc5c(-c6nc(-c7ccc8oc9ncccc9c8c7)nc(-c7ccc(-c8ccccc8)c8ccccc78)n6)cccc45)c3c2)cc1 ; c1cc(-c2ccc(-n3c4ccccc4c4ccc5ccccc5c43)cc2)cc(-c2nc(-c3ccc(-c4ccc5ccccc5c4)cc3)nc(-c3cnc4oc5ccccc5c4c3)n2)c1 ; c1ccc(-c2ccc(-c3nc(-c4cccc(-c5cccc(-n6c7ccccc7c7c8ccccc8ccc76)c5)c4)nc(-c4cc5c(cn4)oc4ccccc45)n3)c3ccccc23)cc1]

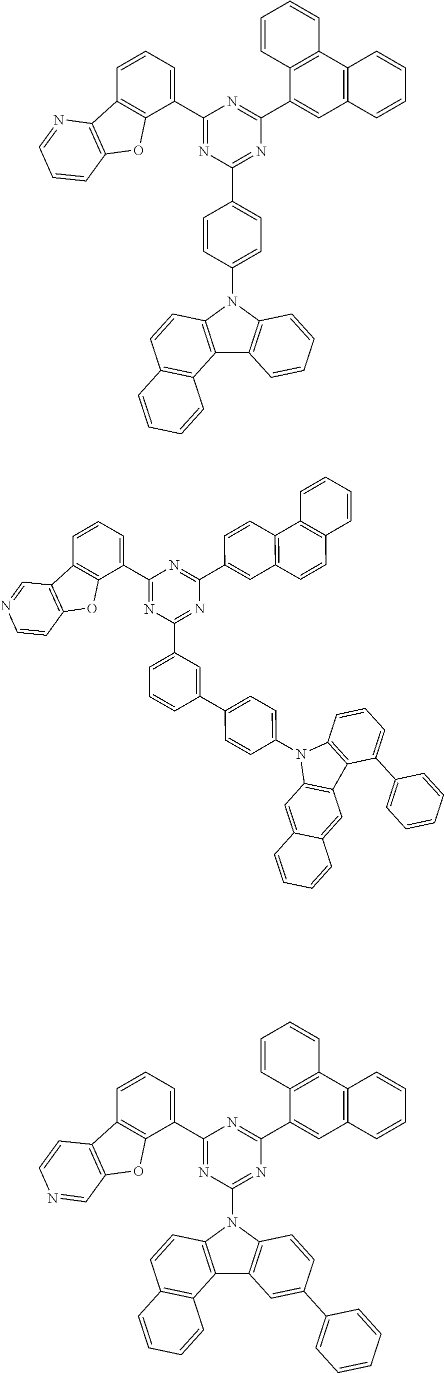 [SMILES: c1ccc2c(c1)cc(-c1nc(-c3ccc(-n4c5ccccc5c5c6ccccc6ccc54)cc3)nc(-c3cccc4c3oc3cccnc34)n1)c1ccccc12 ; c1ccc(-c2cccc3c2c2cc4ccccc4cc2n3-c2ccc(-c3cccc(-c4nc(-c5ccc6c(ccc7ccccc76)c5)nc(-c5cccc6c5oc5ccncc56)n4)c3)cc2)cc1 ; c1ccc(-c2ccc3c(c2)c2c4ccccc4ccc2n3-c2nc(-c3cc4ccccc4c4ccccc34)nc(-c3cccc4c3oc3cnccc34)n2)cc1]

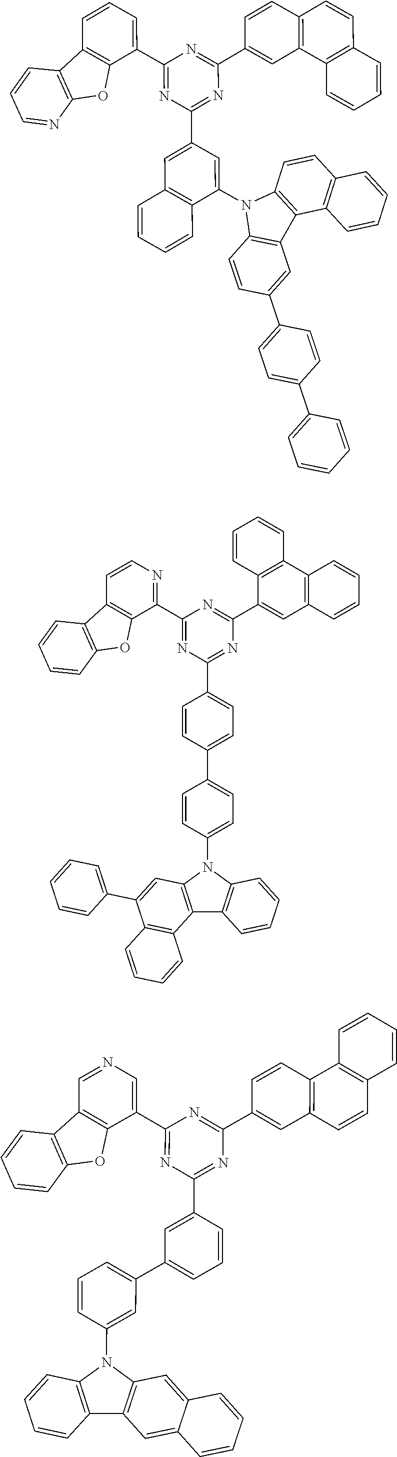 [SMILES: c1ccc(-c2ccc(-c3ccc4c(c3)c3c5ccccc5ccc3n4-c3cc(-c4nc(-c5ccc6ccc7ccccc7c6c5)nc(-c5cccc6c5oc5ncccc56)n4)cc4ccccc34)cc2)cc1 ; c1ccc(-c2cc3c(c4ccccc24)c2ccccc2n3-c2ccc(-c3ccc(-c4nc(-c5cc6ccccc6c6ccccc56)nc(-c5nccc6c5oc5ccccc56)n4)cc3)cc2)cc1 ; c1cc(-c2cccc(-n3c4ccccc4c4cc5ccccc5cc43)c2)cc(-c2nc(-c3ccc4c(ccc5ccccc54)c3)nc(-c3cncc4c3oc3ccccc34)n2)c1]

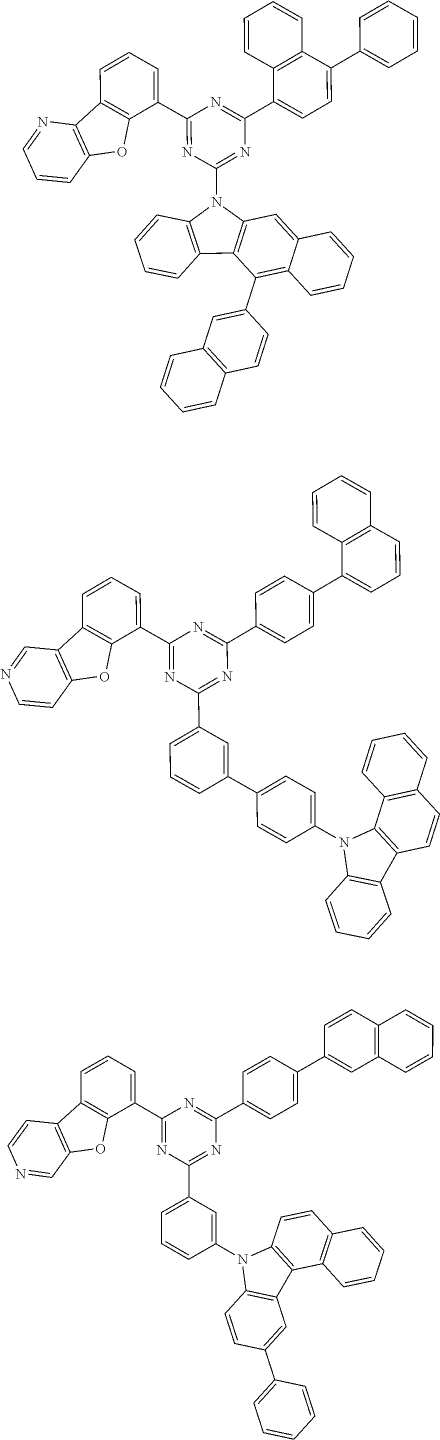 [SMILES: c1ccc(-c2ccc(-c3nc(-c4cccc5c4oc4cccnc45)nc(-n4c5ccccc5c5c(-c6ccc7ccccc7c6)c6ccccc6cc54)n3)c3ccccc23)cc1 ; c1cc(-c2ccc(-n3c4ccccc4c4ccc5ccccc5c43)cc2)cc(-c2nc(-c3ccc(-c4cccc5ccccc45)cc3)nc(-c3cccc4c3oc3ccncc34)n2)c1 ; c1ccc(-c2ccc3c(c2)c2c4ccccc4ccc2n3-c2cccc(-c3nc(-c4ccc(-c5ccc6ccccc6c5)cc4)nc(-c4cccc5c4oc4cnccc45)n3)c2)cc1]

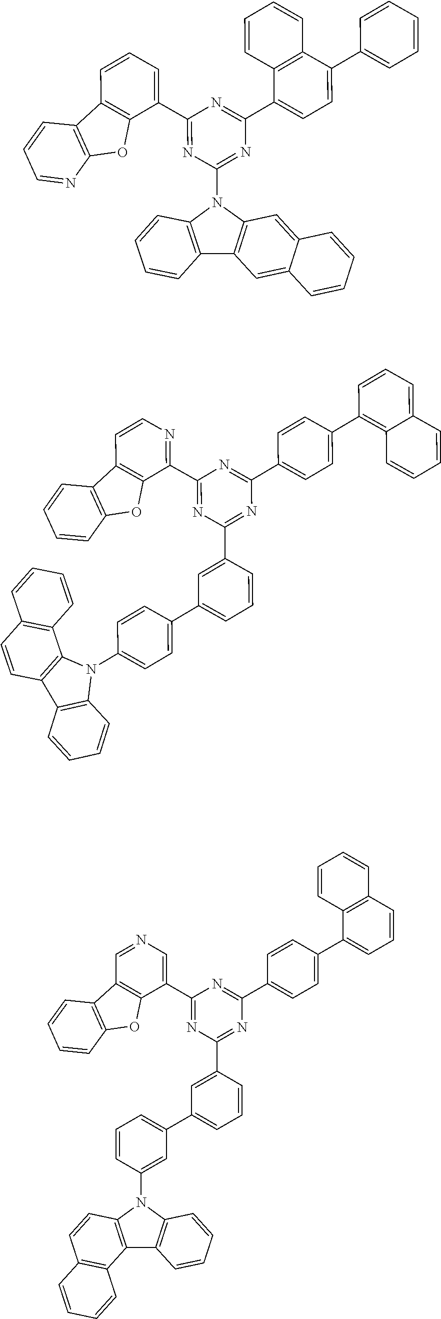 [SMILES: c1ccc(-c2ccc(-c3nc(-c4cccc5c4oc4ncccc45)nc(-n4c5ccccc5c5cc6ccccc6cc54)n3)c3ccccc23)cc1 ; c1cc(-c2ccc(-n3c4ccccc4c4ccc5ccccc5c43)cc2)cc(-c2nc(-c3ccc(-c4cccc5ccccc45)cc3)nc(-c3nccc4c3oc3ccccc34)n2)c1 ; c1cc(-c2cccc(-n3c4ccccc4c4c5ccccc5ccc43)c2)cc(-c2nc(-c3ccc(-c4cccc5ccccc45)cc3)nc(-c3cncc4c3oc3ccccc34)n2)c1]

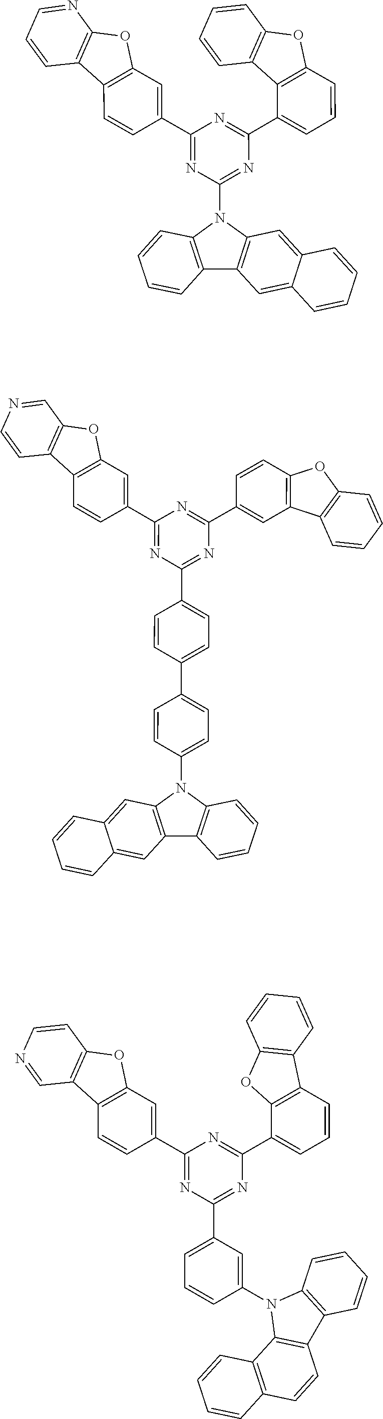 [SMILES: c1ccc2cc3c(cc2c1)c1ccccc1n3-c1nc(-c2ccc3c(c2)oc2ncccc23)nc(-c2cccc3oc4ccccc4c23)n1 ; c1ccc2cc3c(cc2c1)c1ccccc1n3-c1ccc(-c2ccc(-c3nc(-c4ccc5c(c4)oc4cnccc45)nc(-c4ccc5oc6ccccc6c5c4)n3)cc2)cc1 ; c1cc(-c2nc(-c3ccc4c(c3)oc3ccncc34)nc(-c3cccc4c3oc3ccccc34)n2)cc(-n2c3ccccc3c3ccc4ccccc4c32)c1]

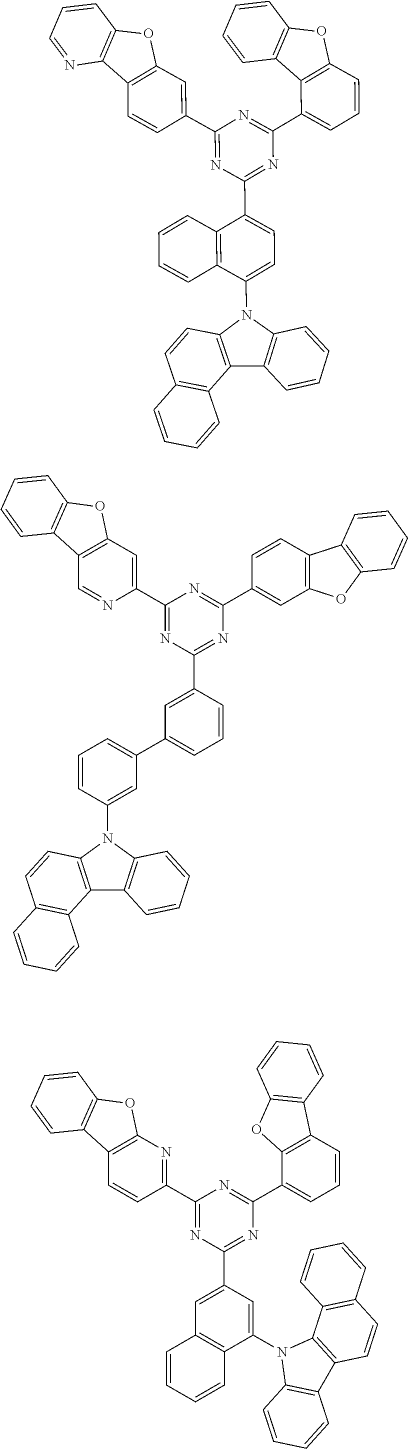 [SMILES: c1ccc2c(c1)ccc1c2c2ccccc2n1-c1ccc(-c2nc(-c3ccc4c(c3)oc3cccnc34)nc(-c3cccc4oc5ccccc5c34)n2)c2ccccc12 ; c1cc(-c2cccc(-n3c4ccccc4c4c5ccccc5ccc43)c2)cc(-c2nc(-c3ccc4c(c3)oc3ccccc34)nc(-c3cc4oc5ccccc5c4cn3)n2)c1 ; c1ccc2c(-n3c4ccccc4c4ccc5ccccc5c43)cc(-c3nc(-c4ccc5c(n4)oc4ccccc45)nc(-c4cccc5c4oc4ccccc45)n3)cc2c1]

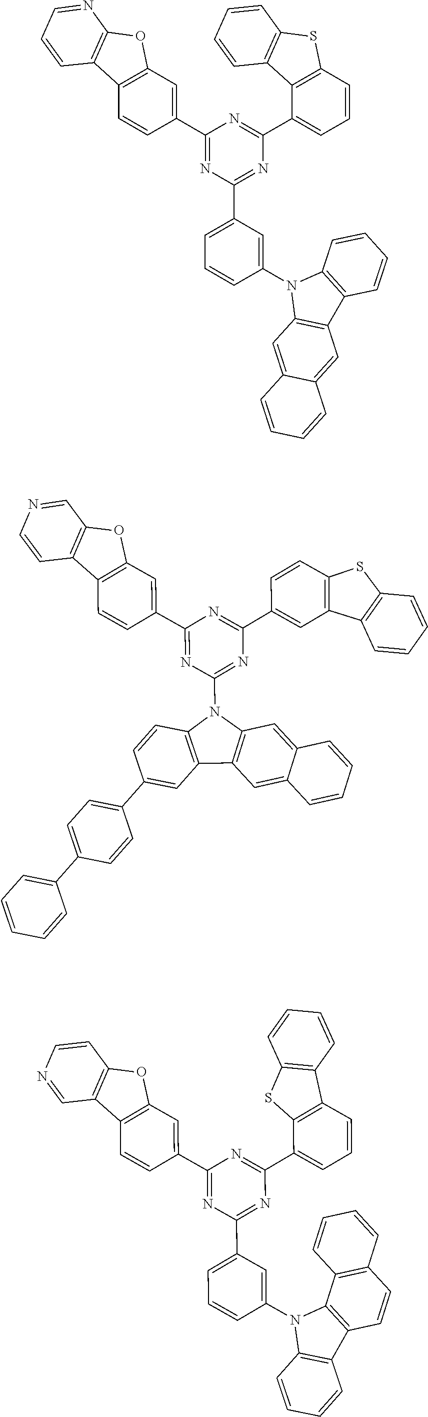 [SMILES: c1cc(-c2nc(-c3ccc4c(c3)oc3ncccc34)nc(-c3cccc4sc5ccccc5c34)n2)cc(-n2c3ccccc3c3cc4ccccc4cc32)c1 ; c1ccc(-c2ccc(-c3ccc4c(c3)c3cc5ccccc5cc3n4-c3nc(-c4ccc5c(c4)oc4cnccc45)nc(-c4ccc5sc6ccccc6c5c4)n3)cc2)cc1 ; c1cc(-c2nc(-c3ccc4c(c3)oc3ccncc34)nc(-c3cccc4c3sc3ccccc34)n2)cc(-n2c3ccccc3c3ccc4ccccc4c32)c1]

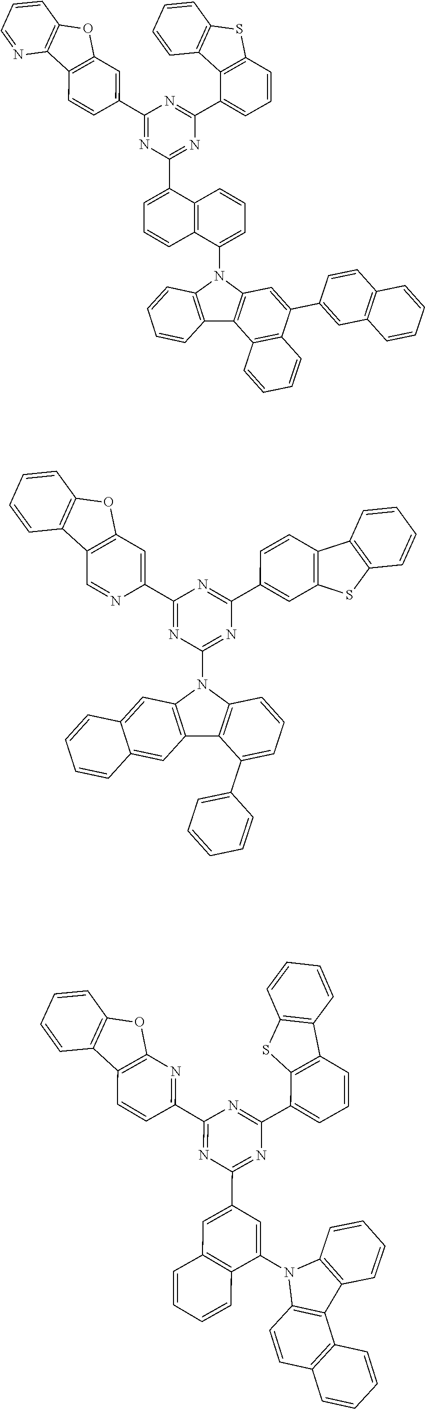 [SMILES: c1ccc2cc(-c3cc4c(c5ccccc35)c3ccccc3n4-c3cccc4c(-c5nc(-c6ccc7c(c6)oc6cccnc67)nc(-c6cccc7sc8ccccc8c67)n5)cccc34)ccc2c1 ; c1ccc(-c2cccc3c2c2cc4ccccc4cc2n3-c2nc(-c3ccc4c(c3)sc3ccccc34)nc(-c3cc4oc5ccccc5c4cn3)n2)cc1 ; c1ccc2c(-n3c4ccccc4c4c5ccccc5ccc43)cc(-c3nc(-c4ccc5c(n4)oc4ccccc45)nc(-c4cccc5c4sc4ccccc45)n3)cc2c1]

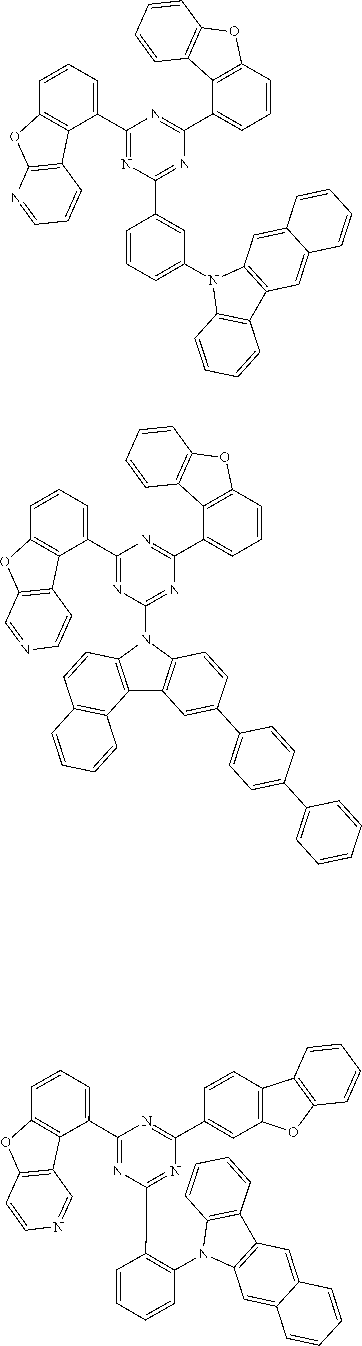 [SMILES: c1cc(-c2nc(-c3cccc4oc5ccccc5c34)nc(-c3cccc4oc5ncccc5c34)n2)cc(-n2c3ccccc3c3cc4ccccc4cc32)c1 ; c1ccc(-c2ccc(-c3ccc4c(c3)c3c5ccccc5ccc3n4-c3nc(-c4cccc5oc6ccccc6c45)nc(-c4cccc5oc6cnccc6c45)n3)cc2)cc1 ; c1ccc(-n2c3ccccc3c3cc4ccccc4cc32)c(-c2nc(-c3ccc4c(c3)oc3ccccc34)nc(-c3cccc4oc5ccncc5c34)n2)c1]

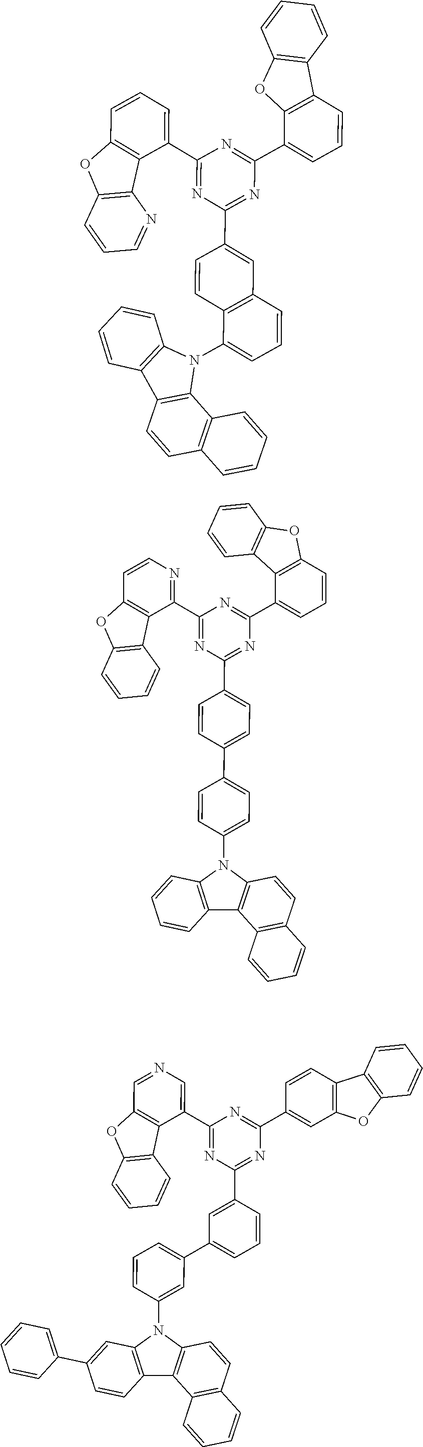

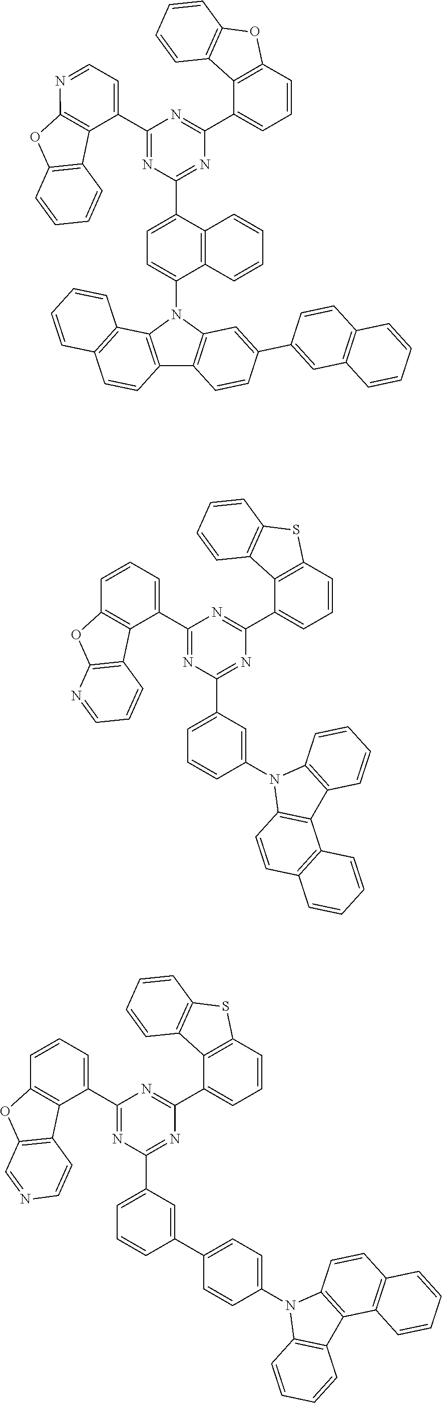 [SMILES: c1ccc2cc(-c3ccc4c5ccc6ccccc6c5n(-c5ccc(-c6nc(-c7cccc8oc9ccccc9c78)nc(-c7ccnc8oc9ccccc9c78)n6)c6ccccc56)c4c3)ccc2c1 ; c1cc(-c2nc(-c3cccc4oc5ncccc5c34)nc(-c3cccc4sc5ccccc5c34)n2)cc(-n2c3ccccc3c3c4ccccc4ccc32)c1 ; c1cc(-c2ccc(-n3c4ccccc4c4c5ccccc5ccc43)cc2)cc(-c2nc(-c3cccc4oc5cnccc5c34)nc(-c3cccc4sc5ccccc5c34)n2)c1]

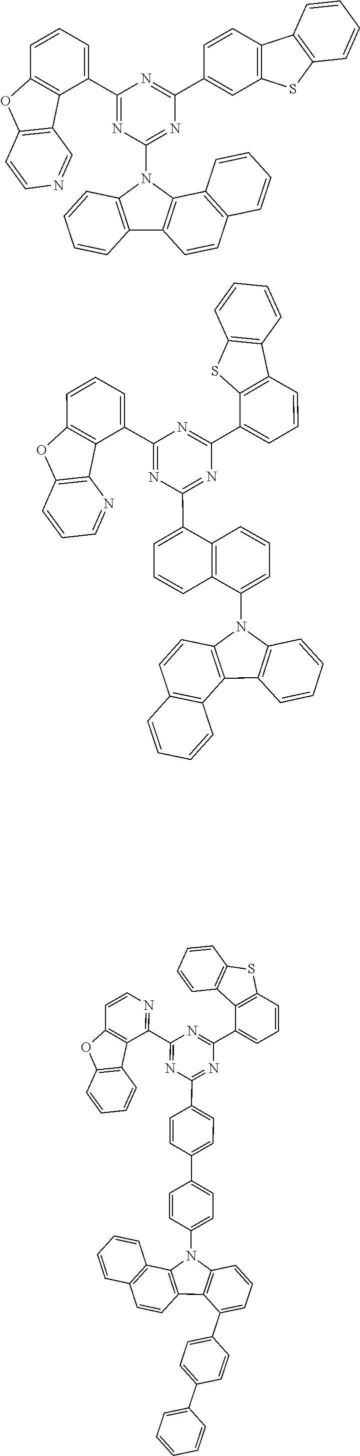

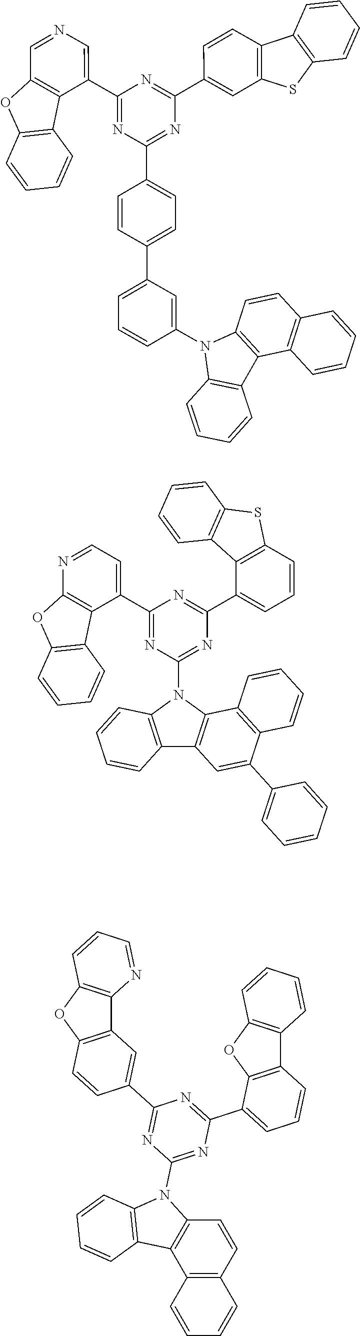

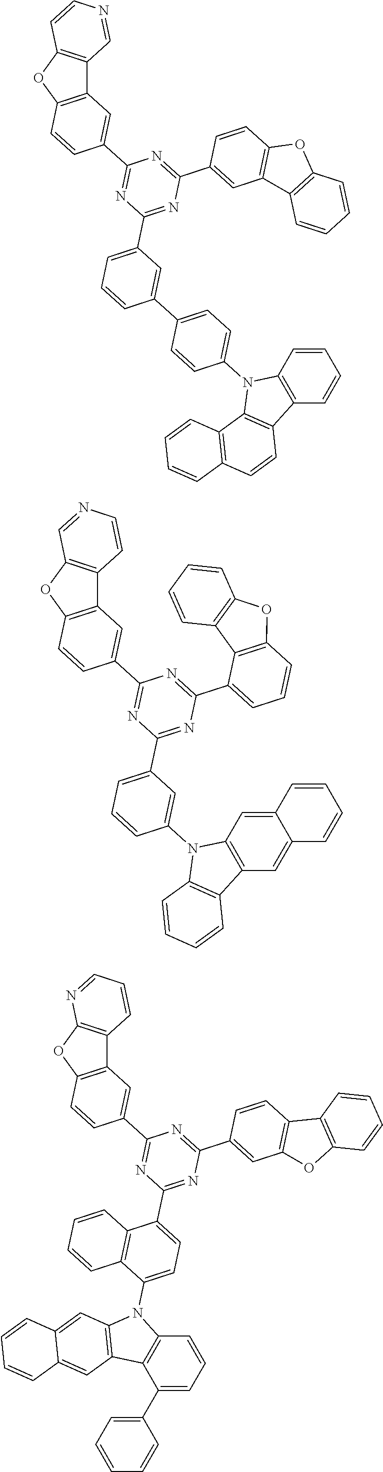 [SMILES: c1cc(-c2ccc(-n3c4ccccc4c4ccc5ccccc5c43)cc2)cc(-c2nc(-c3ccc4oc5ccccc5c4c3)nc(-c3ccc4oc5ccncc5c4c3)n2)c1 ; c1cc(-c2nc(-c3ccc4oc5cnccc5c4c3)nc(-c3cccc4oc5ccccc5c34)n2)cc(-n2c3ccccc3c3cc4ccccc4cc32)c1 ; c1ccc(-c2cccc3c2c2cc4ccccc4cc2n3-c2ccc(-c3nc(-c4ccc5c(c4)oc4ccccc45)nc(-c4ccc5oc6ncccc6c5c4)n3)c3ccccc23)cc1]

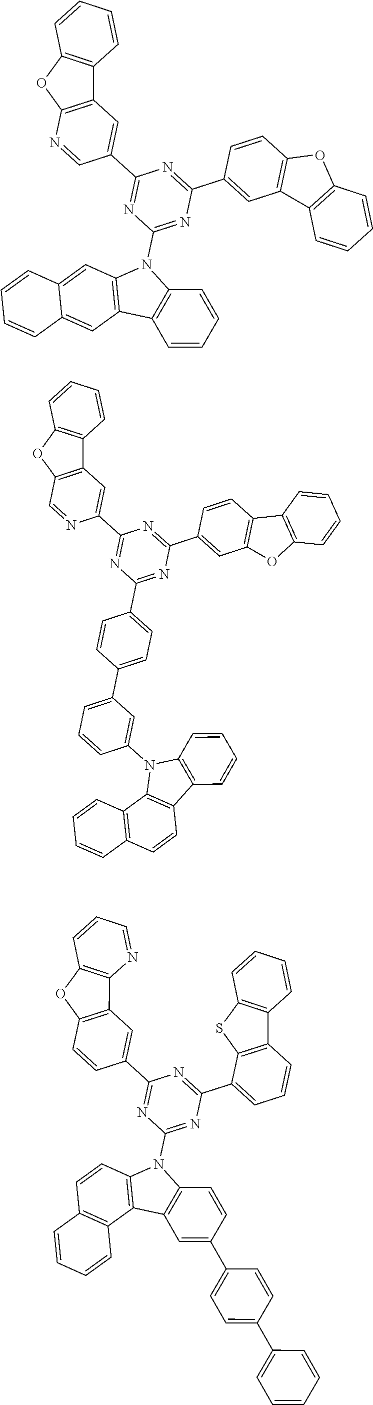 [SMILES: c1ccc2cc3c(cc2c1)c1ccccc1n3-c1nc(-c2ccc3oc4ccccc4c3c2)nc(-c2cnc3oc4ccccc4c3c2)n1 ; c1cc(-c2ccc(-c3nc(-c4ccc5c(c4)oc4ccccc45)nc(-c4cc5c(cn4)oc4ccccc45)n3)cc2)cc(-n2c3ccccc3c3ccc4ccccc4c32)c1 ; c1ccc(-c2ccc(-c3ccc4c(c3)c3c5ccccc5ccc3n4-c3nc(-c4ccc5oc6cccnc6c5c4)nc(-c4cccc5c4sc4ccccc45)n3)cc2)cc1]

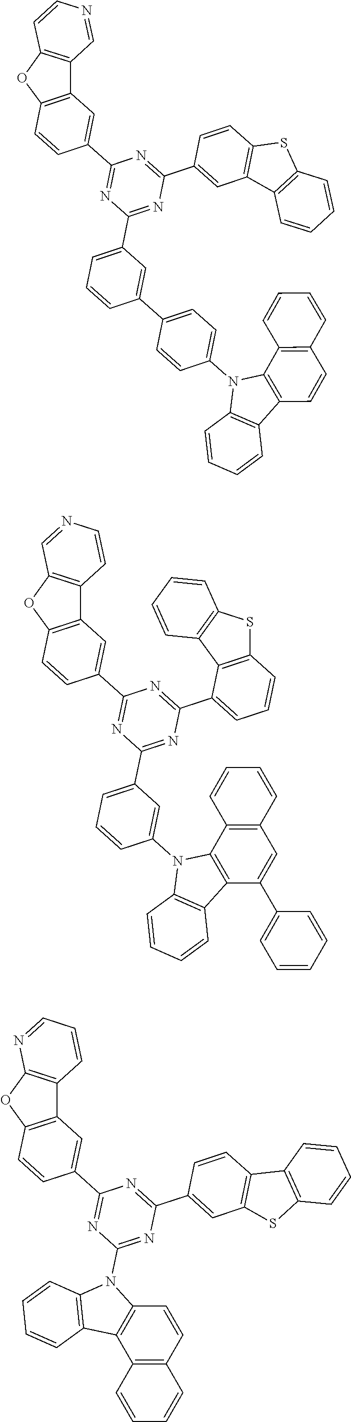 [SMILES: c1cc(-c2ccc(-n3c4ccccc4c4ccc5ccccc5c43)cc2)cc(-c2nc(-c3ccc4oc5ccncc5c4c3)nc(-c3ccc4sc5ccccc5c4c3)n2)c1 ; c1ccc(-c2cc3ccccc3c3c2c2ccccc2n3-c2cccc(-c3nc(-c4ccc5oc6cnccc6c5c4)nc(-c4cccc5sc6ccccc6c45)n3)c2)cc1 ; c1ccc2c(c1)ccc1c2c2ccccc2n1-c1nc(-c2ccc3c(c2)sc2ccccc23)nc(-c2ccc3oc4ncccc4c3c2)n1]

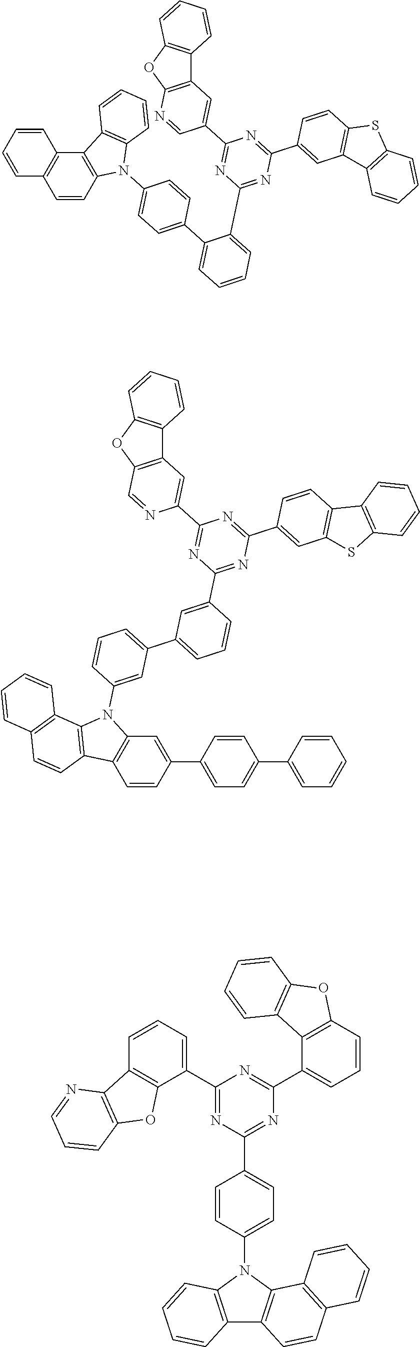 [SMILES: c1ccc(-c2nc(-c3cnc4oc5ccccc5c4c3)nc(-c3ccc4sc5ccccc5c4c3)n2)c(-c2ccc(-n3c4ccccc4c4c5ccccc5ccc43)cc2)c1 ; c1ccc(-c2ccc(-c3ccc4c5ccc6ccccc6c5n(-c5cccc(-c6cccc(-c7nc(-c8ccc9c(c8)sc8ccccc89)nc(-c8cc9c(cn8)oc8ccccc89)n7)c6)c5)c4c3)cc2)cc1 ; c1ccc2c(c1)ccc1c3ccccc3n(-c3ccc(-c4nc(-c5cccc6c5oc5cccnc56)nc(-c5cccc6oc7ccccc7c56)n4)cc3)c21]

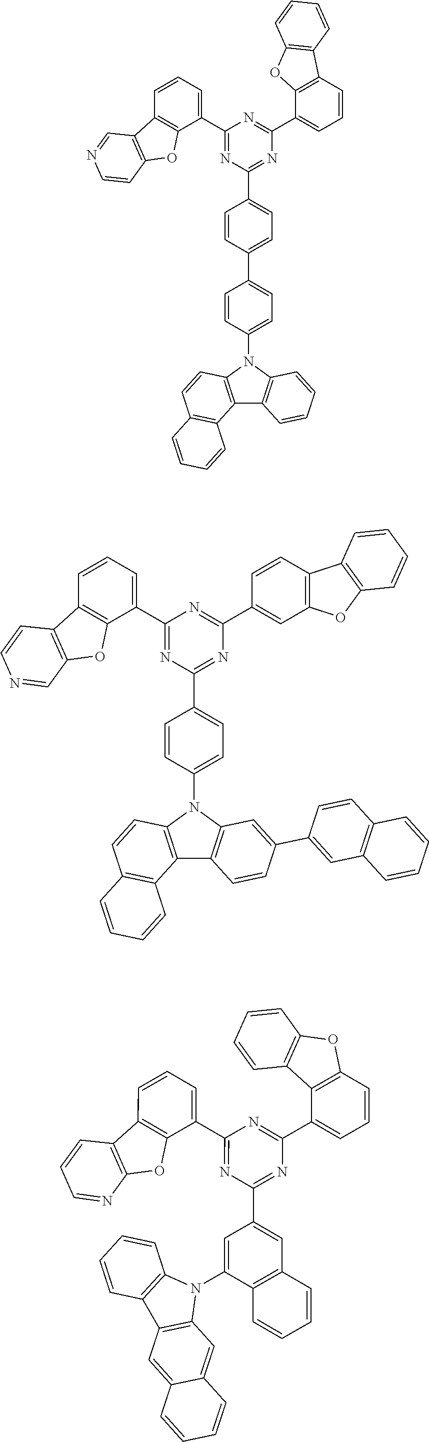 [SMILES: c1ccc2c(c1)ccc1c2c2ccccc2n1-c1ccc(-c2ccc(-c3nc(-c4cccc5c4oc4ccccc45)nc(-c4cccc5c4oc4ccncc45)n3)cc2)cc1 ; c1ccc2cc(-c3ccc4c5c6ccccc6ccc5n(-c5ccc(-c6nc(-c7ccc8c(c7)oc7ccccc78)nc(-c7cccc8c7oc7cnccc78)n6)cc5)c4c3)ccc2c1 ; c1ccc2cc3c(cc2c1)c1ccccc1n3-c1cc(-c2nc(-c3cccc4c3oc3ncccc34)nc(-c3cccc4oc5ccccc5c34)n2)cc2ccccc12]

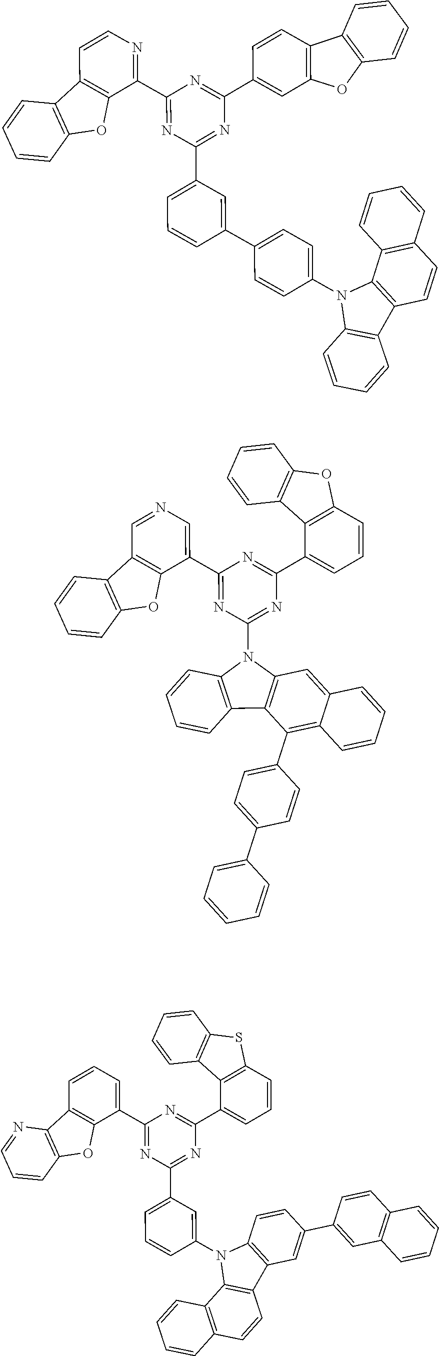 [SMILES: c1cc(-c2ccc(-n3c4ccccc4c4ccc5ccccc5c43)cc2)cc(-c2nc(-c3ccc4c(c3)oc3ccccc34)nc(-c3nccc4c3oc3ccccc34)n2)c1 ; c1ccc(-c2ccc(-c3c4ccccc4cc4c3c3ccccc3n4-c3nc(-c4cncc5c4oc4ccccc45)nc(-c4cccc5oc6ccccc6c45)n3)cc2)cc1 ; c1cc(-c2nc(-c3cccc4c3oc3cccnc34)nc(-c3cccc4sc5ccccc5c34)n2)cc(-n2c3ccc(-c4ccc5ccccc5c4)cc3c3ccc4ccccc4c32)c1]

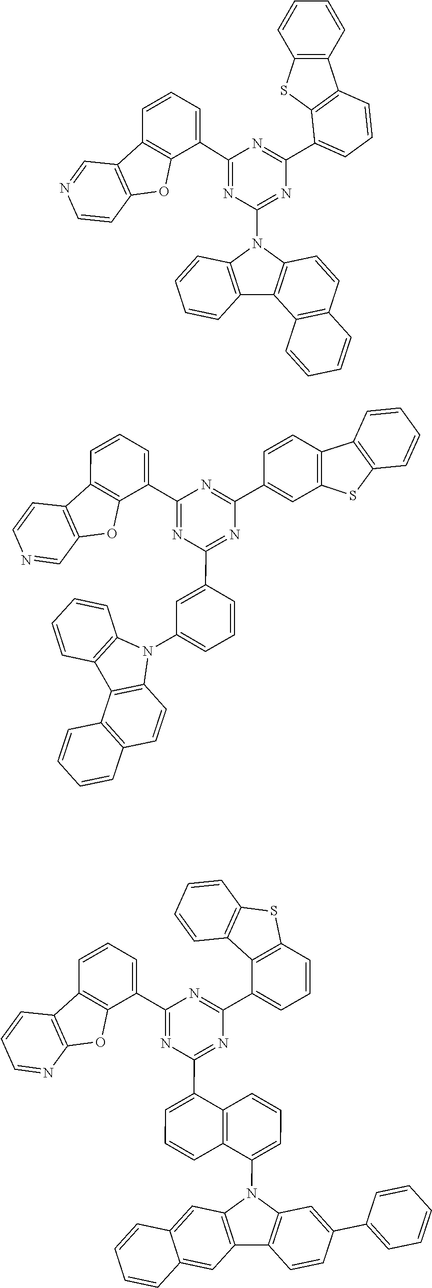 [SMILES: c1ccc2c(c1)ccc1c2c2ccccc2n1-c1nc(-c2cccc3c2oc2ccncc23)nc(-c2cccc3c2sc2ccccc23)n1 ; c1cc(-c2nc(-c3ccc4c(c3)sc3ccccc34)nc(-c3cccc4c3oc3cnccc34)n2)cc(-n2c3ccccc3c3c4ccccc4ccc32)c1 ; c1ccc(-c2ccc3c4cc5ccccc5cc4n(-c4cccc5c(-c6nc(-c7cccc8c7oc7ncccc78)nc(-c7cccc8sc9ccccc9c78)n6)cccc45)c3c2)cc1]

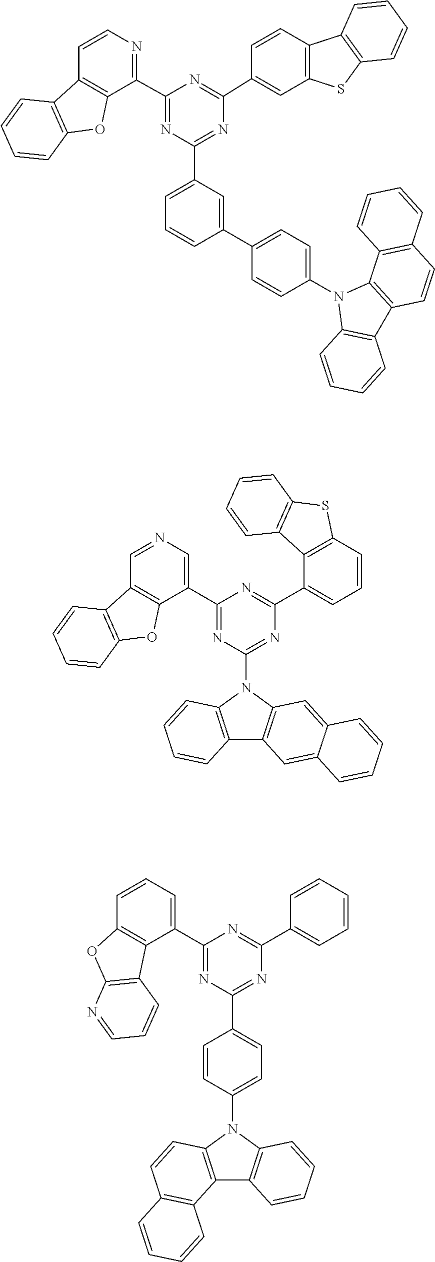 [SMILES: c1cc(-c2ccc(-n3c4ccccc4c4ccc5ccccc5c43)cc2)cc(-c2nc(-c3ccc4c(c3)sc3ccccc34)nc(-c3nccc4c3oc3ccccc34)n2)c1 ; c1ccc2cc3c(cc2c1)c1ccccc1n3-c1nc(-c2cncc3c2oc2ccccc23)nc(-c2cccc3sc4ccccc4c23)n1 ; c1ccc(-c2nc(-c3ccc(-n4c5ccccc5c5c6ccccc6ccc54)cc3)nc(-c3cccc4oc5ncccc5c34)n2)cc1]

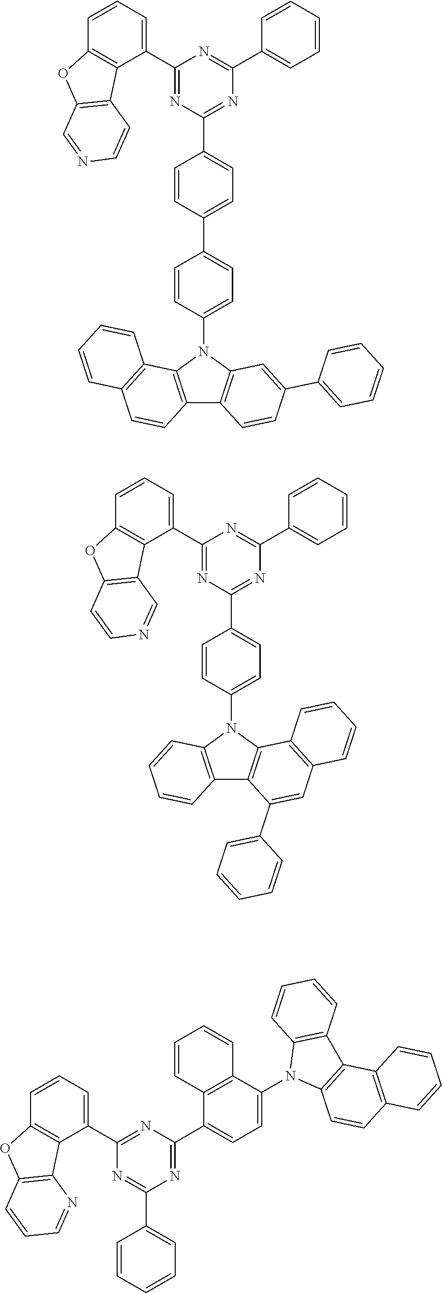 [SMILES: c1ccc(-c2ccc3c4ccc5ccccc5c4n(-c4ccc(-c5ccc(-c6nc(-c7ccccc7)nc(-c7cccc8oc9cnccc9c78)n6)cc5)cc4)c3c2)cc1 ; c1ccc(-c2nc(-c3ccc(-n4c5ccccc5c5c(-c6ccccc6)cc6ccccc6c54)cc3)nc(-c3cccc4oc5ccncc5c34)n2)cc1 ; c1ccc(-c2nc(-c3ccc(-n4c5ccccc5c5c6ccccc6ccc54)c4ccccc34)nc(-c3cccc4oc5cccnc5c34)n2)cc1]

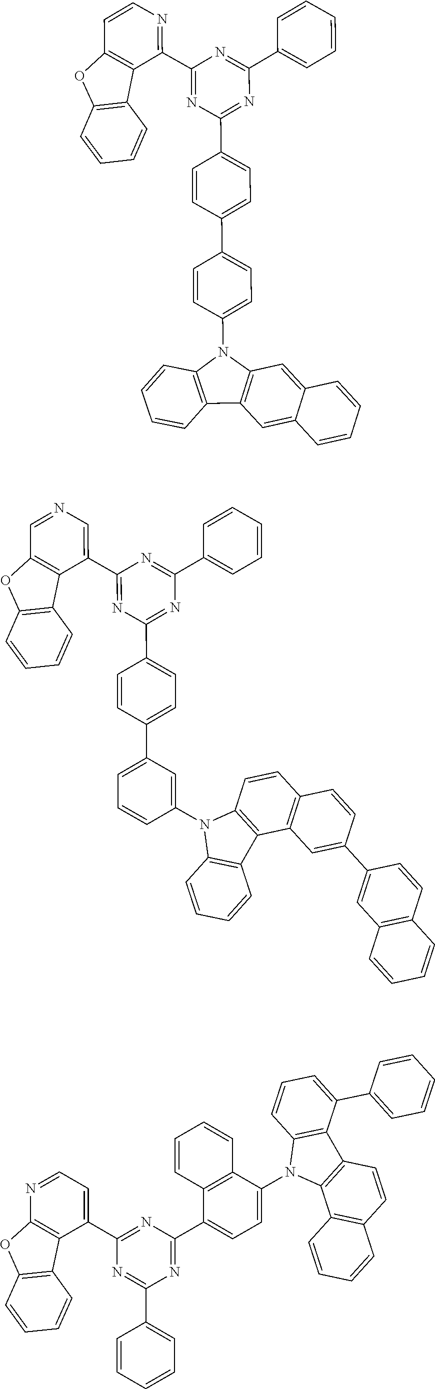 [SMILES: c1ccc(-c2nc(-c3ccc(-c4ccc(-n5c6ccccc6c6cc7ccccc7cc65)cc4)cc3)nc(-c3nccc4oc5ccccc5c34)n2)cc1 ; c1ccc(-c2nc(-c3ccc(-c4cccc(-n5c6ccccc6c6c7cc(-c8ccc9ccccc9c8)ccc7ccc65)c4)cc3)nc(-c3cncc4oc5ccccc5c34)n2)cc1 ; c1ccc(-c2nc(-c3ccc(-n4c5cccc(-c6ccccc6)c5c5ccc6ccccc6c54)c4ccccc34)nc(-c3ccnc4oc5ccccc5c34)n2)cc1]

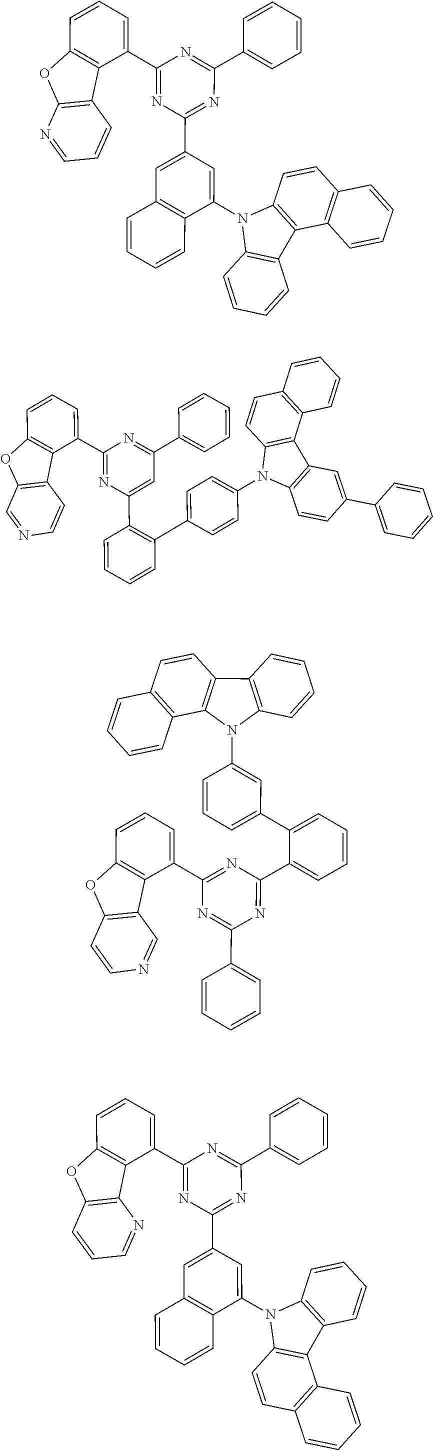 [SMILES: c1ccc(-c2nc(-c3cc(-n4c5ccccc5c5c6ccccc6ccc54)c4ccccc4c3)nc(-c3cccc4oc5ncccc5c34)n2)cc1 ; c1ccc(-c2ccc3c(c2)c2c4ccccc4ccc2n3-c2ccc(-c3ccccc3-c3cc(-c4ccccc4)nc(-c4cccc5oc6cnccc6c45)n3)cc2)cc1 ; c1ccc(-c2nc(-c3ccccc3-c3cccc(-n4c5ccccc5c5ccc6ccccc6c54)c3)nc(-c3cccc4oc5ccncc5c34)n2)cc1 ; c1ccc(-c2nc(-c3cc(-n4c5ccccc5c5c6ccccc6ccc54)c4ccccc4c3)nc(-c3cccc4oc5cccnc5c34)n2)cc1]

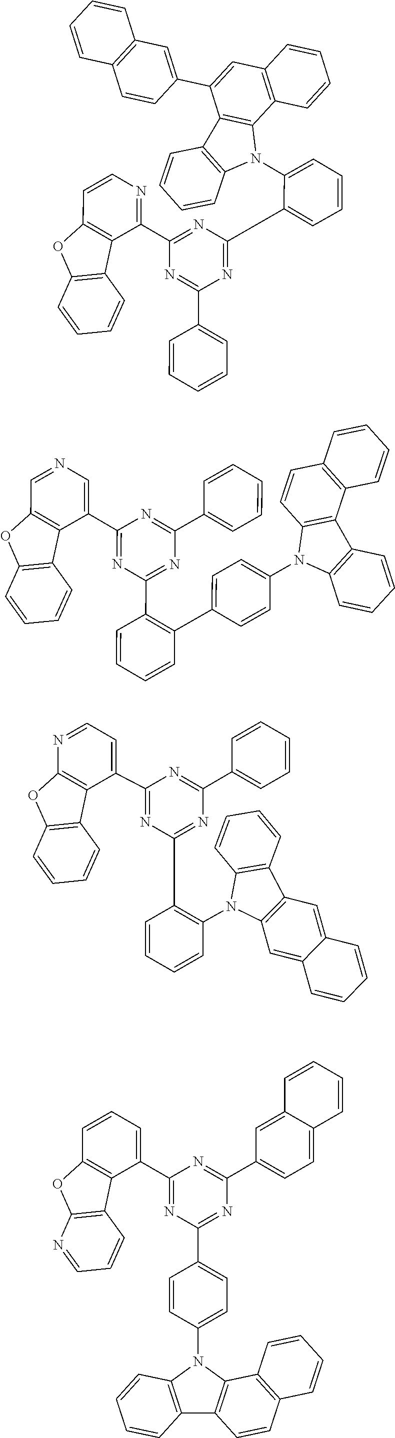 [SMILES: c1ccc(-c2nc(-c3ccccc3-n3c4ccccc4c4c(-c5ccc6ccccc6c5)cc5ccccc5c43)nc(-c3nccc4oc5ccccc5c34)n2)cc1 ; c1ccc(-c2nc(-c3ccccc3-c3ccc(-n4c5ccccc5c5c6ccccc6ccc54)cc3)nc(-c3cncc4oc5ccccc5c34)n2)cc1 ; c1ccc(-c2nc(-c3ccccc3-n3c4ccccc4c4cc5ccccc5cc43)nc(-c3ccnc4oc5ccccc5c34)n2)cc1 ; c1ccc2cc(-c3nc(-c4ccc(-n5c6ccccc6c6ccc7ccccc7c65)cc4)nc(-c4cccc5oc6ncccc6c45)n3)ccc2c1]

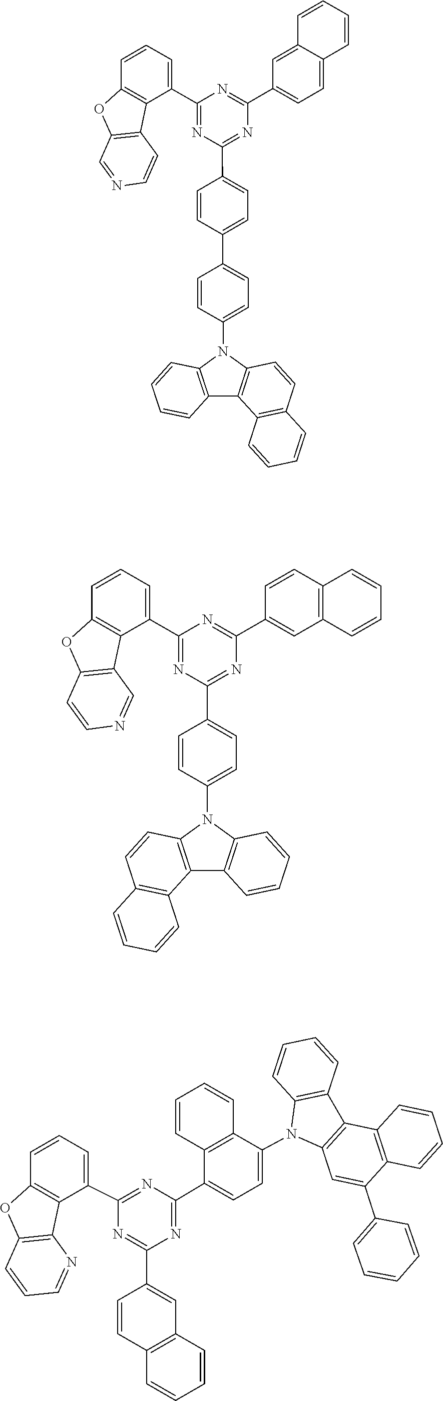 [SMILES: c1ccc2cc(-c3nc(-c4ccc(-c5ccc(-n6c7ccccc7c7c8ccccc8ccc76)cc5)cc4)nc(-c4cccc5oc6cnccc6c45)n3)ccc2c1 ; c1ccc2cc(-c3nc(-c4ccc(-n5c6ccccc6c6c7ccccc7ccc65)cc4)nc(-c4cccc5oc6ccncc6c45)n3)ccc2c1 ; c1ccc(-c2cc3c(c4ccccc24)c2ccccc2n3-c2ccc(-c3nc(-c4ccc5ccccc5c4)nc(-c4cccc5oc6cccnc6c45)n3)c3ccccc23)cc1]

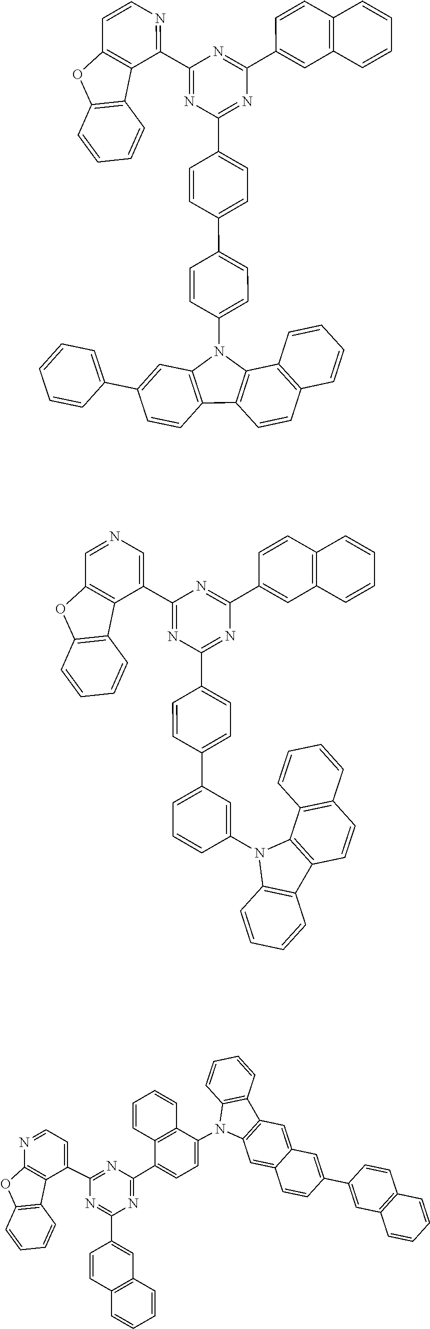 [SMILES: c1ccc(-c2ccc3c4ccc5ccccc5c4n(-c4ccc(-c5ccc(-c6nc(-c7ccc8ccccc8c7)nc(-c7nccc8oc9ccccc9c78)n6)cc5)cc4)c3c2)cc1 ; c1cc(-c2ccc(-c3nc(-c4ccc5ccccc5c4)nc(-c4cncc5oc6ccccc6c45)n3)cc2)cc(-n2c3ccccc3c3ccc4ccccc4c32)c1 ; c1ccc2cc(-c3ccc4cc5c(cc4c3)c3ccccc3n5-c3ccc(-c4nc(-c5ccc6ccccc6c5)nc(-c5ccnc6oc7ccccc7c56)n4)c4ccccc34)ccc2c1]

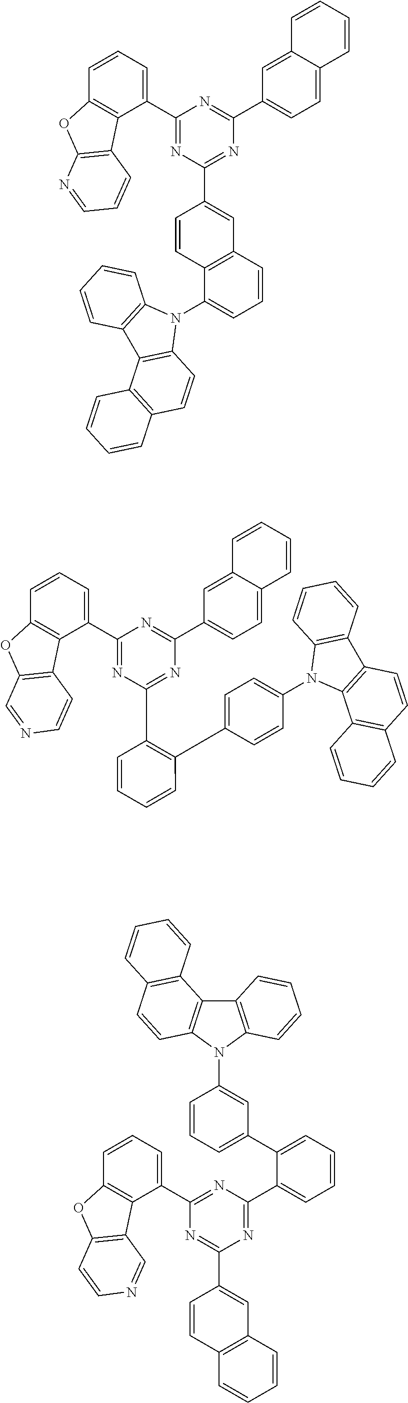 [SMILES: c1ccc2cc(-c3nc(-c4ccc5c(-n6c7ccccc7c7c8ccccc8ccc76)cccc5c4)nc(-c4cccc5oc6ncccc6c45)n3)ccc2c1 ; c1ccc(-c2nc(-c3ccc4ccccc4c3)nc(-c3cccc4oc5cnccc5c34)n2)c(-c2ccc(-n3c4ccccc4c4ccc5ccccc5c43)cc2)c1 ; c1cc(-c2ccccc2-c2nc(-c3ccc4ccccc4c3)nc(-c3cccc4oc5ccncc5c34)n2)cc(-n2c3ccccc3c3c4ccccc4ccc32)c1]

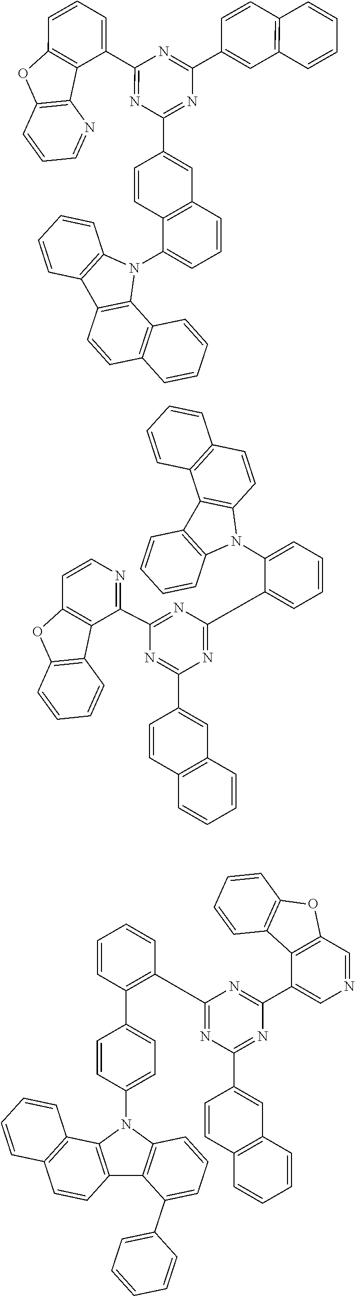

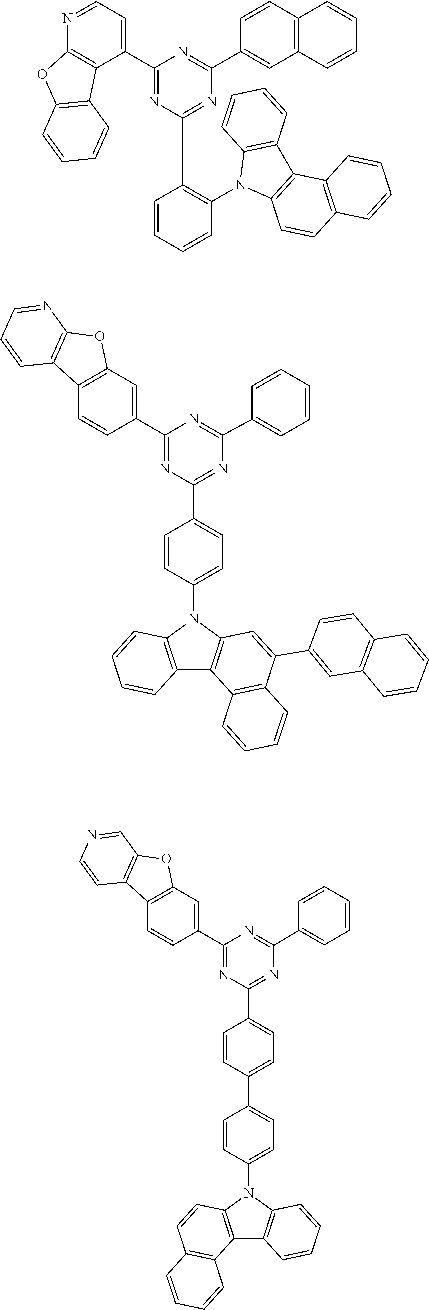 [SMILES: c1ccc(-n2c3ccccc3c3c4ccccc4ccc32)c(-c2nc(-c3ccc4ccccc4c3)nc(-c3ccnc4oc5ccccc5c34)n2)c1 ; c1ccc(-c2nc(-c3ccc(-n4c5ccccc5c5c6ccccc6c(-c6ccc7ccccc7c6)cc54)cc3)nc(-c3ccc4c(c3)oc3ncccc34)n2)cc1 ; c1ccc(-c2nc(-c3ccc(-c4ccc(-n5c6ccccc6c6c7ccccc7ccc65)cc4)cc3)nc(-c3ccc4c(c3)oc3cnccc34)n2)cc1]

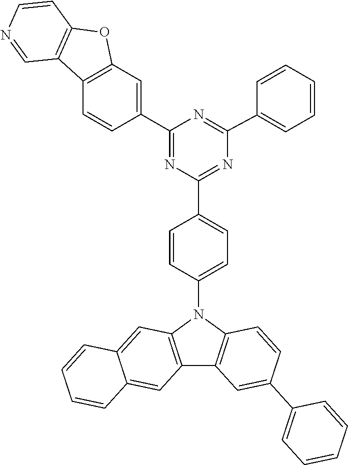 [SMILES: c1ccc(-c2ccc3c(c2)c2cc4ccccc4cc2n3-c2ccc(-c3nc(-c4ccccc4)nc(-c4ccc5c(c4)oc4ccncc45)n3)cc2)cc1]

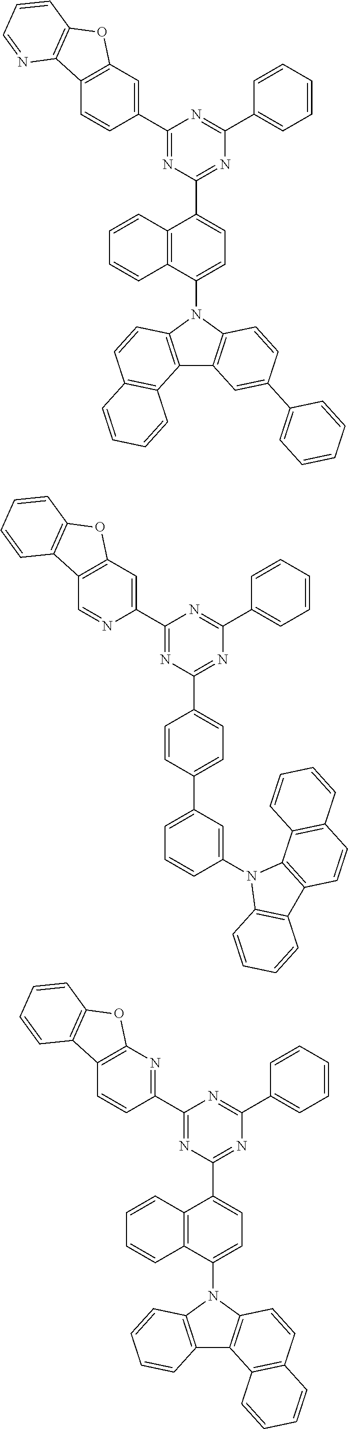 [SMILES: c1ccc(-c2ccc3c(c2)c2c4ccccc4ccc2n3-c2ccc(-c3nc(-c4ccccc4)nc(-c4ccc5c(c4)oc4cccnc45)n3)c3ccccc23)cc1 ; c1ccc(-c2nc(-c3ccc(-c4cccc(-n5c6ccccc6c6ccc7ccccc7c65)c4)cc3)nc(-c3cc4oc5ccccc5c4cn3)n2)cc1 ; c1ccc(-c2nc(-c3ccc4c(n3)oc3ccccc34)nc(-c3ccc(-n4c5ccccc5c5c6ccccc6ccc54)c4ccccc34)n2)cc1]

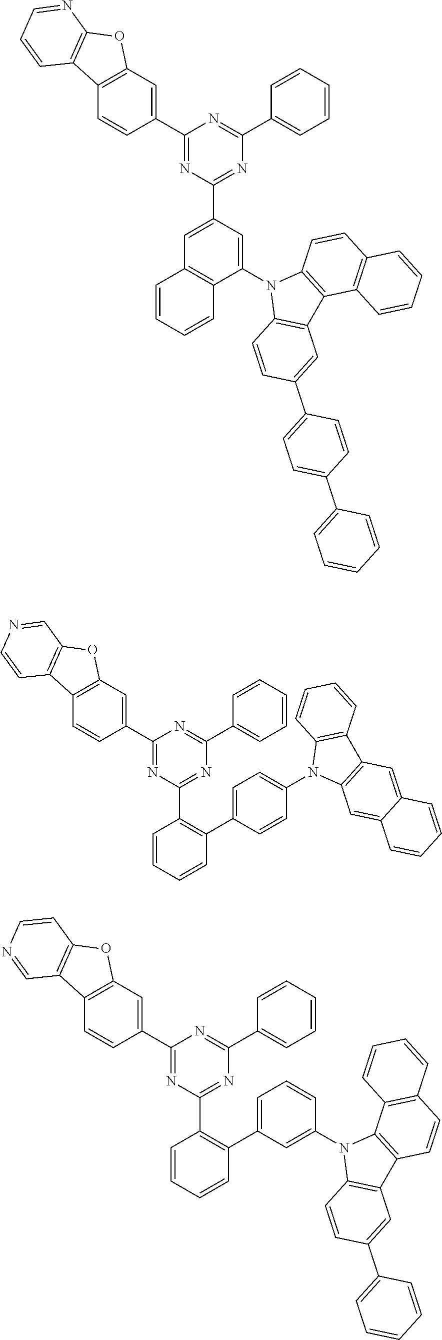 [SMILES: c1ccc(-c2ccc(-c3ccc4c(c3)c3c5ccccc5ccc3n4-c3cc(-c4nc(-c5ccccc5)nc(-c5ccc6c(c5)oc5ncccc56)n4)cc4ccccc34)cc2)cc1 ; c1ccc(-c2nc(-c3ccc4c(c3)oc3cnccc34)nc(-c3ccccc3-c3ccc(-n4c5ccccc5c5cc6ccccc6cc54)cc3)n2)cc1 ; c1ccc(-c2ccc3c(c2)c2ccc4ccccc4c2n3-c2cccc(-c3ccccc3-c3nc(-c4ccccc4)nc(-c4ccc5c(c4)oc4ccncc45)n3)c2)cc1]

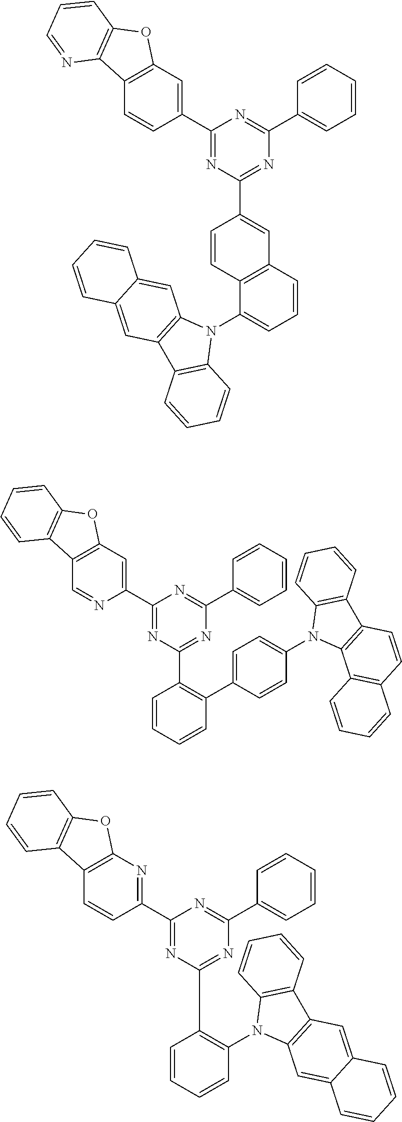 [SMILES: c1ccc(-c2nc(-c3ccc4c(-n5c6ccccc6c6cc7ccccc7cc65)cccc4c3)nc(-c3ccc4c(c3)oc3cccnc34)n2)cc1 ; c1ccc(-c2nc(-c3cc4oc5ccccc5c4cn3)nc(-c3ccccc3-c3ccc(-n4c5ccccc5c5ccc6ccccc6c54)cc3)n2)cc1 ; c1ccc(-c2nc(-c3ccc4c(n3)oc3ccccc34)nc(-c3ccccc3-n3c4ccccc4c4cc5ccccc5cc43)n2)cc1]

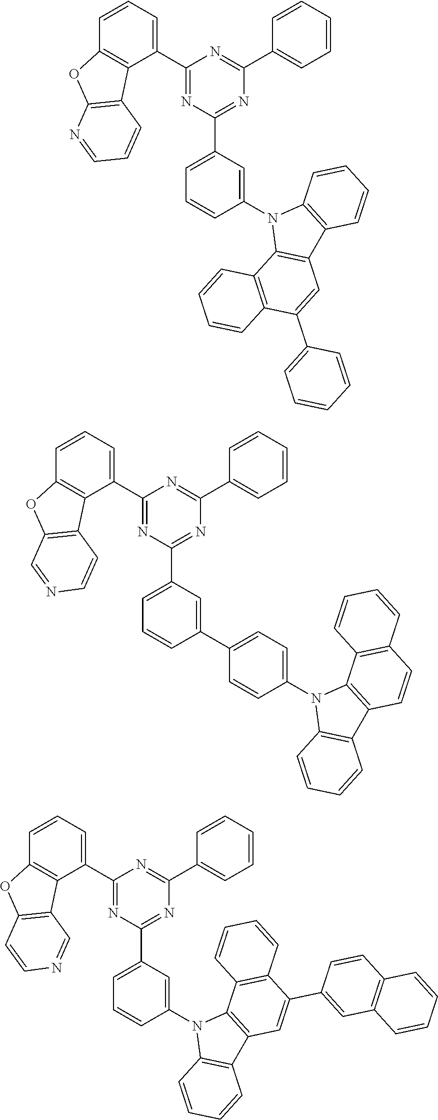

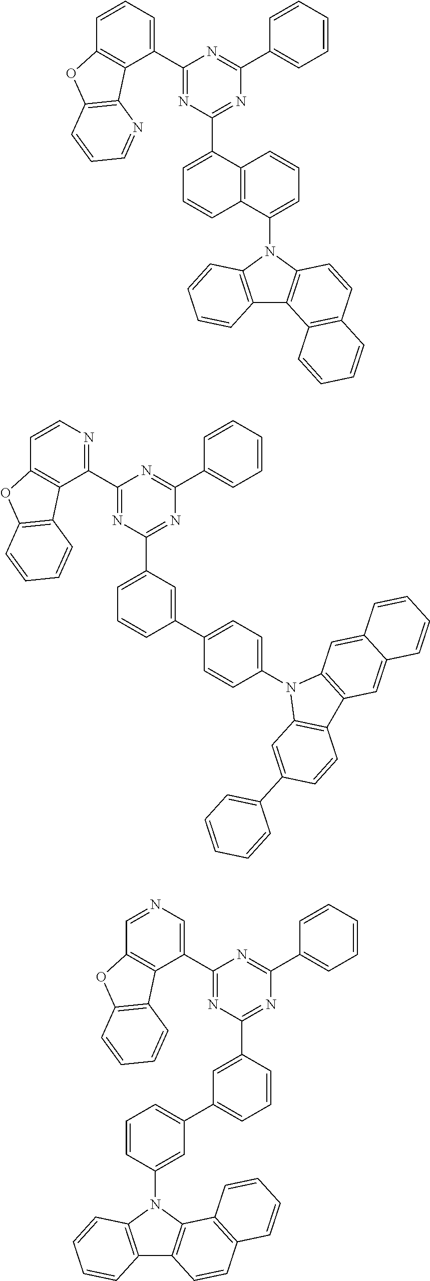 [SMILES: c1ccc(-c2nc(-c3cccc4c(-n5c6ccccc6c6c7ccccc7ccc65)cccc34)nc(-c3cccc4oc5cccnc5c34)n2)cc1 ; c1ccc(-c2ccc3c4cc5ccccc5cc4n(-c4ccc(-c5cccc(-c6nc(-c7ccccc7)nc(-c7nccc8oc9ccccc9c78)n6)c5)cc4)c3c2)cc1 ; c1ccc(-c2nc(-c3cccc(-c4cccc(-n5c6ccccc6c6ccc7ccccc7c65)c4)c3)nc(-c3cncc4oc5ccccc5c34)n2)cc1]

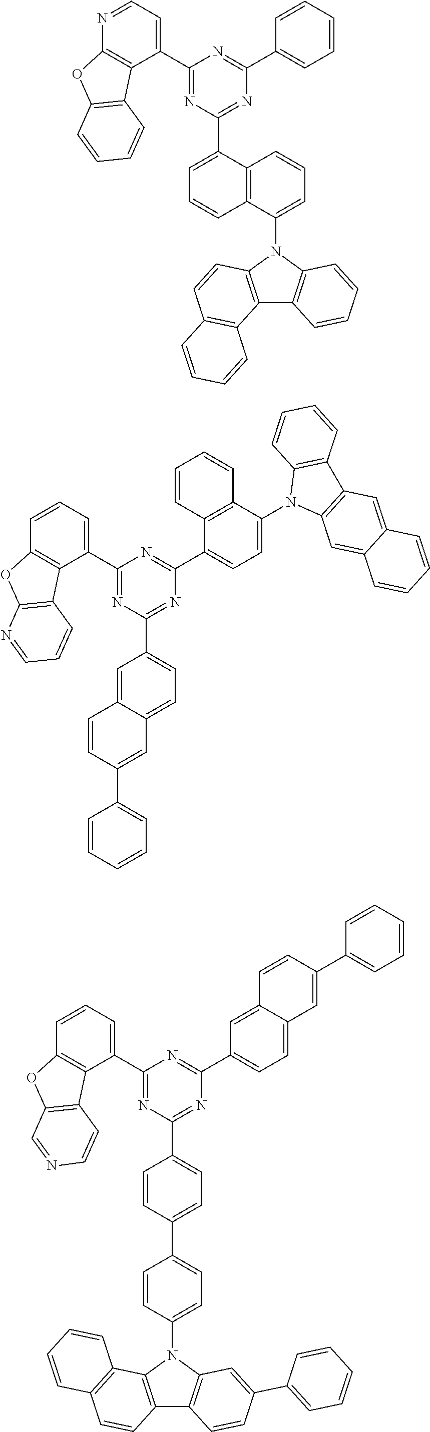 [SMILES: c1ccc(-c2nc(-c3cccc4c(-n5c6ccccc6c6c7ccccc7ccc65)cccc34)nc(-c3ccnc4oc5ccccc5c34)n2)cc1 ; c1ccc(-c2ccc3cc(-c4nc(-c5ccc(-n6c7ccccc7c7cc8ccccc8cc76)c6ccccc56)nc(-c5cccc6oc7ncccc7c56)n4)ccc3c2)cc1 ; c1ccc(-c2ccc3cc(-c4nc(-c5ccc(-c6ccc(-n7c8cc(-c9ccccc9)ccc8c8ccc9ccccc9c87)cc6)cc5)nc(-c5cccc6oc7cnccc7c56)n4)ccc3c2)cc1]

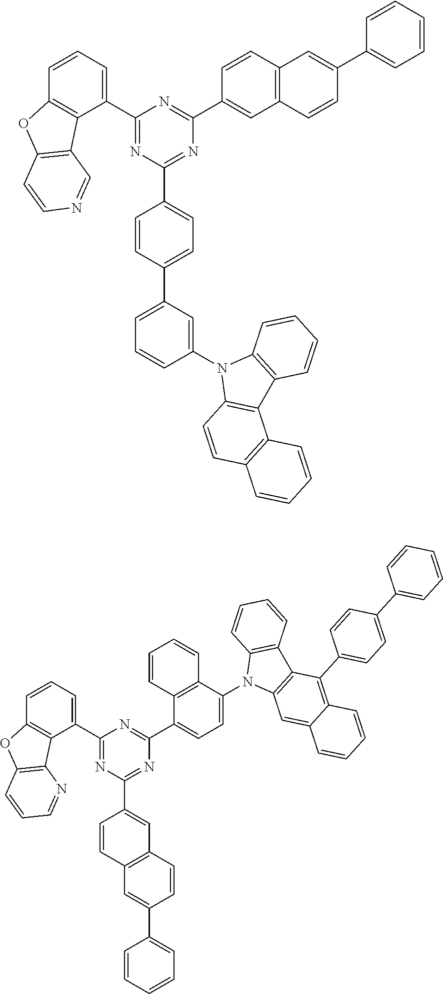 [SMILES: c1ccc(-c2ccc3cc(-c4nc(-c5ccc(-c6cccc(-n7c8ccccc8c8c9ccccc9ccc87)c6)cc5)nc(-c5cccc6oc7ccncc7c56)n4)ccc3c2)cc1 ; c1ccc(-c2ccc(-c3c4ccccc4cc4c3c3ccccc3n4-c3ccc(-c4nc(-c5ccc6cc(-c7ccccc7)ccc6c5)nc(-c5cccc6oc7cccnc7c56)n4)c4ccccc34)cc2)cc1]

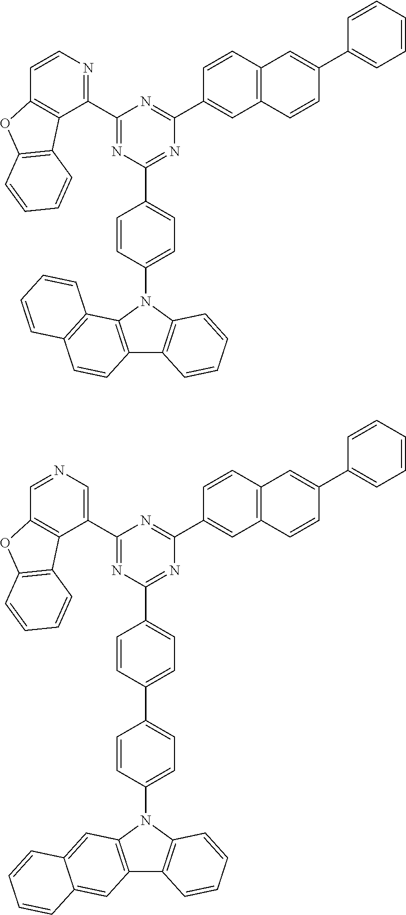 [SMILES: c1ccc(-c2ccc3cc(-c4nc(-c5ccc(-n6c7ccccc7c7ccc8ccccc8c76)cc5)nc(-c5nccc6oc7ccccc7c56)n4)ccc3c2)cc1 ; c1ccc(-c2ccc3cc(-c4nc(-c5ccc(-c6ccc(-n7c8ccccc8c8cc9ccccc9cc87)cc6)cc5)nc(-c5cncc6oc7ccccc7c56)n4)ccc3c2)cc1]

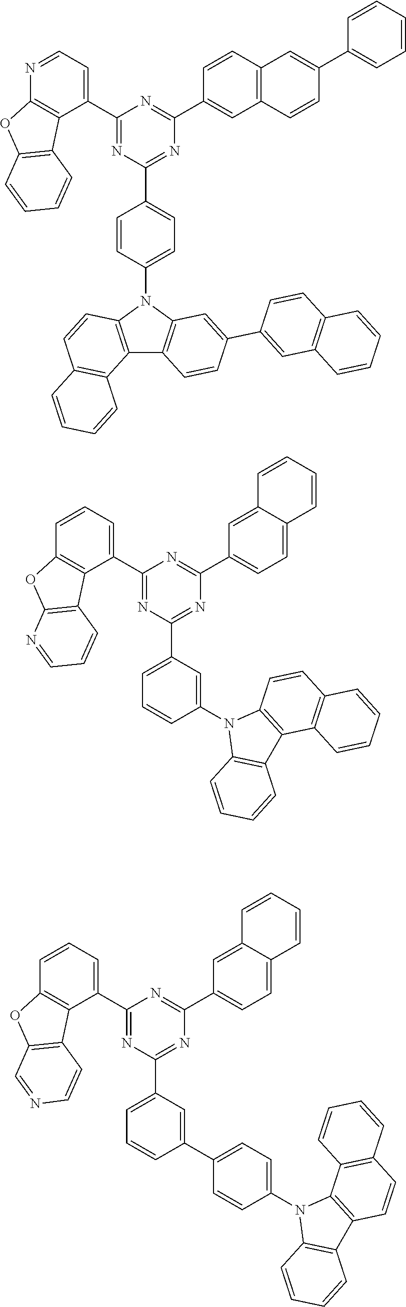 [SMILES: c1ccc(-c2ccc3cc(-c4nc(-c5ccc(-n6c7cc(-c8ccc9ccccc9c8)ccc7c7c8ccccc8ccc76)cc5)nc(-c5ccnc6oc7ccccc7c56)n4)ccc3c2)cc1 ; c1cc(-c2nc(-c3ccc4ccccc4c3)nc(-c3cccc4oc5ncccc5c34)n2)cc(-n2c3ccccc3c3c4ccccc4ccc32)c1 ; c1cc(-c2ccc(-n3c4ccccc4c4ccc5ccccc5c43)cc2)cc(-c2nc(-c3ccc4ccccc4c3)nc(-c3cccc4oc5cnccc5c34)n2)c1]

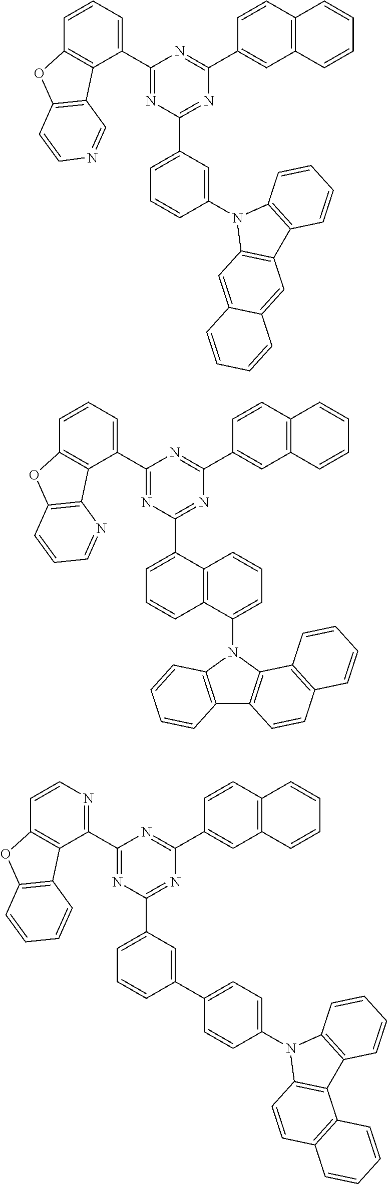

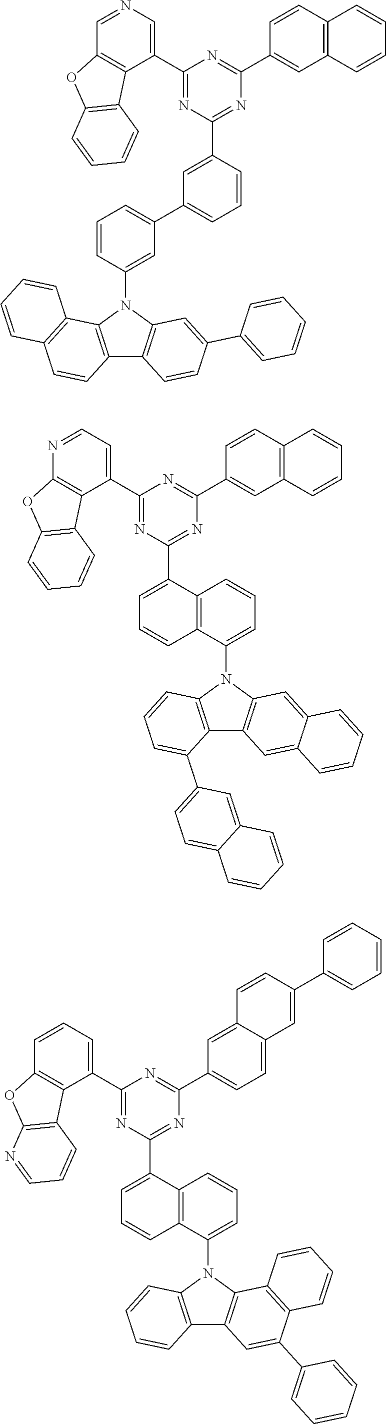 [SMILES: c1ccc(-c2ccc3c4ccc5ccccc5c4n(-c4cccc(-c5cccc(-c6nc(-c7ccc8ccccc8c7)nc(-c7cncc8oc9ccccc9c78)n6)c5)c4)c3c2)cc1 ; c1ccc2cc(-c3nc(-c4cccc5c(-n6c7cc8ccccc8cc7c7c(-c8ccc9ccccc9c8)cccc76)cccc45)nc(-c4ccnc5oc6ccccc6c45)n3)ccc2c1 ; c1ccc(-c2ccc3cc(-c4nc(-c5cccc6c(-n7c8ccccc8c8cc(-c9ccccc9)c9ccccc9c87)cccc56)nc(-c5cccc6oc7ncccc7c56)n4)ccc3c2)cc1]

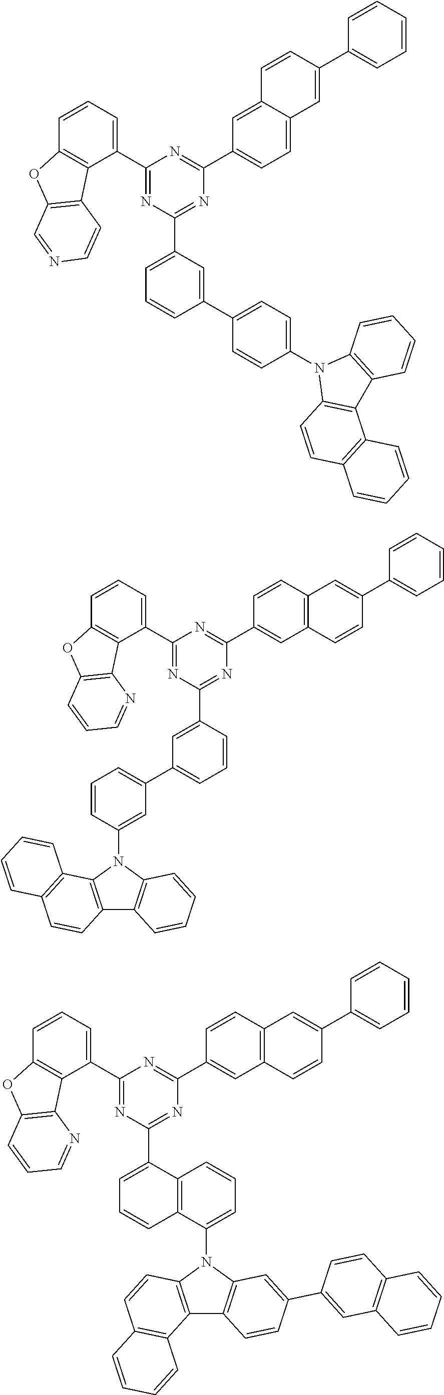

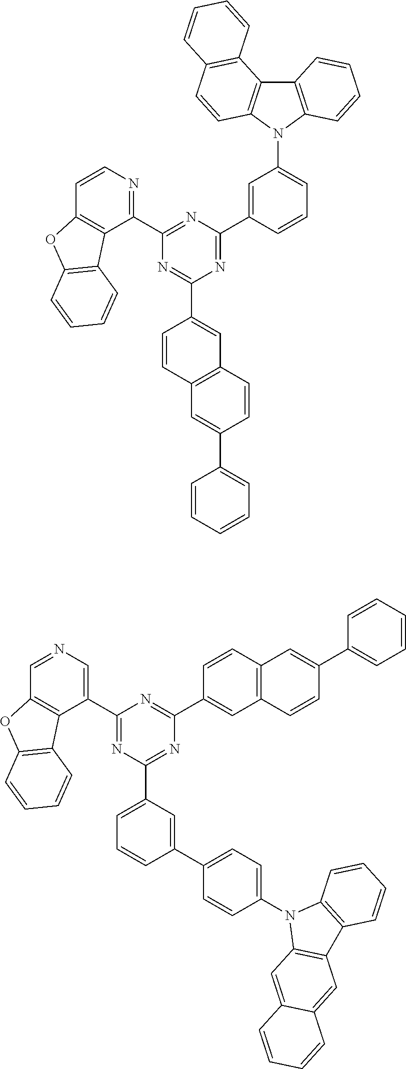 [SMILES: c1ccc(-c2ccc3cc(-c4nc(-c5cccc(-n6c7ccccc7c7c8ccccc8ccc76)c5)nc(-c5nccc6oc7ccccc7c56)n4)ccc3c2)cc1 ; c1ccc(-c2ccc3cc(-c4nc(-c5cccc(-c6ccc(-n7c8ccccc8c8cc9ccccc9cc87)cc6)c5)nc(-c5cncc6oc7ccccc7c56)n4)ccc3c2)cc1]

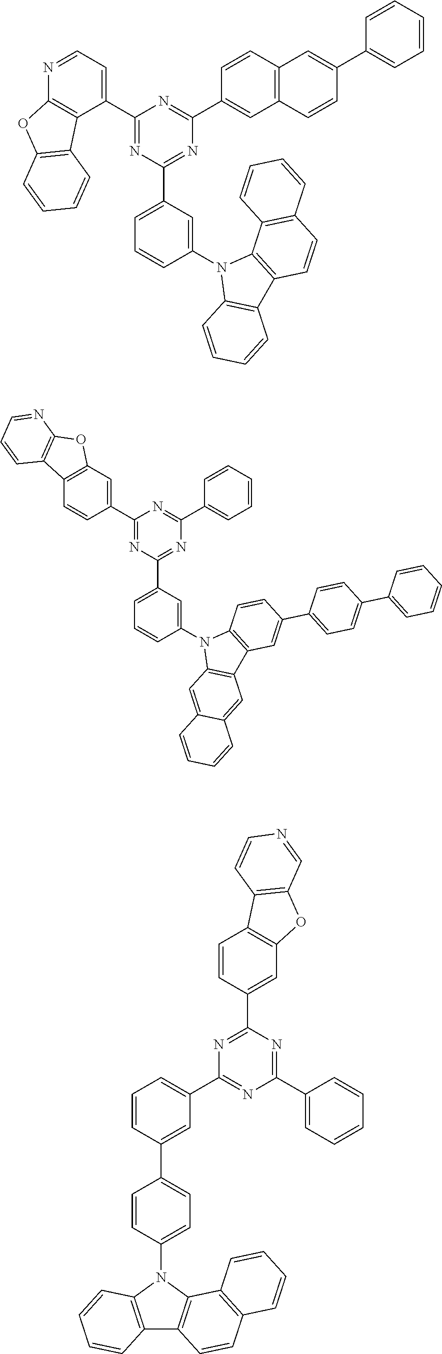 [SMILES: c1ccc(-c2ccc3cc(-c4nc(-c5cccc(-n6c7ccccc7c7ccc8ccccc8c76)c5)nc(-c5ccnc6oc7ccccc7c56)n4)ccc3c2)cc1 ; c1ccc(-c2ccc(-c3ccc4c(c3)c3cc5ccccc5cc3n4-c3cccc(-c4nc(-c5ccccc5)nc(-c5ccc6c(c5)oc5ncccc56)n4)c3)cc2)cc1 ; c1ccc(-c2nc(-c3cccc(-c4ccc(-n5c6ccccc6c6ccc7ccccc7c65)cc4)c3)nc(-c3ccc4c(c3)oc3cnccc34)n2)cc1]

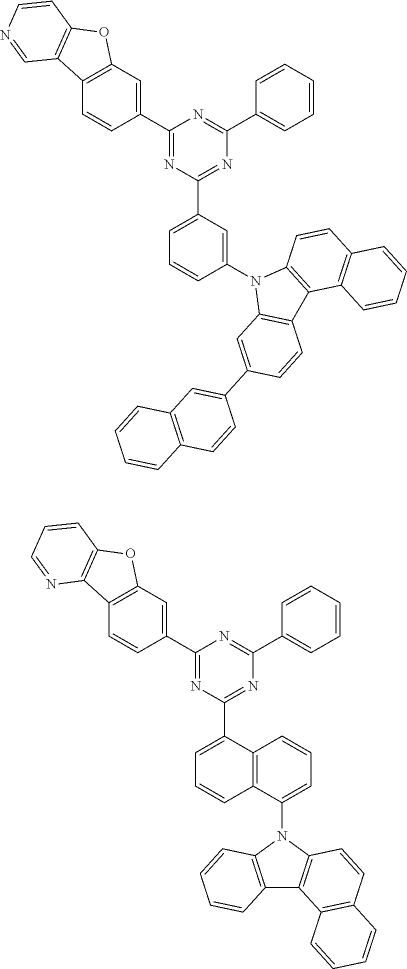 [SMILES: c1ccc(-c2nc(-c3cccc(-n4c5cc(-c6ccc7ccccc7c6)ccc5c5c6ccccc6ccc54)c3)nc(-c3ccc4c(c3)oc3ccncc34)n2)cc1 ; c1ccc(-c2nc(-c3ccc4c(c3)oc3cccnc34)nc(-c3cccc4c(-n5c6ccccc6c6c7ccccc7ccc65)cccc34)n2)cc1]

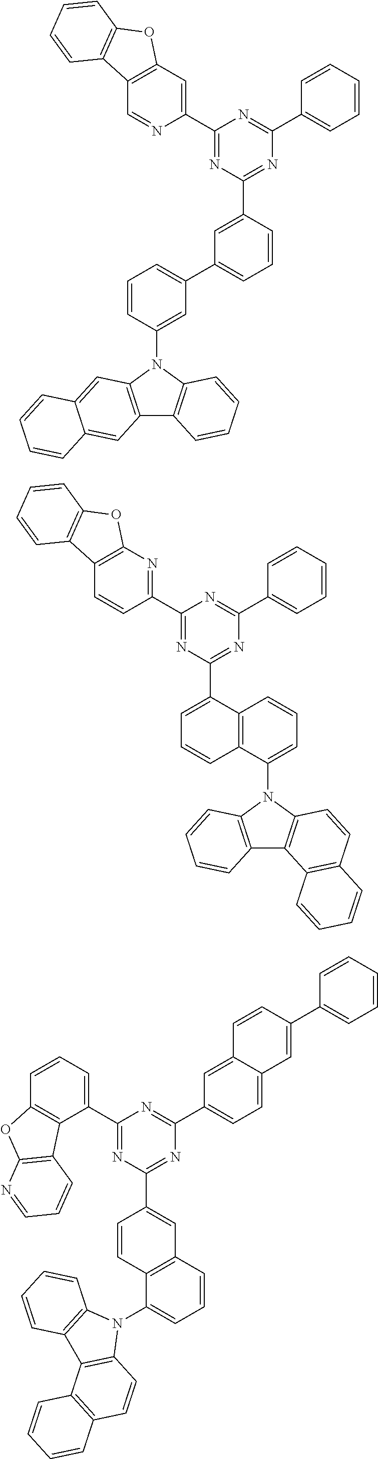

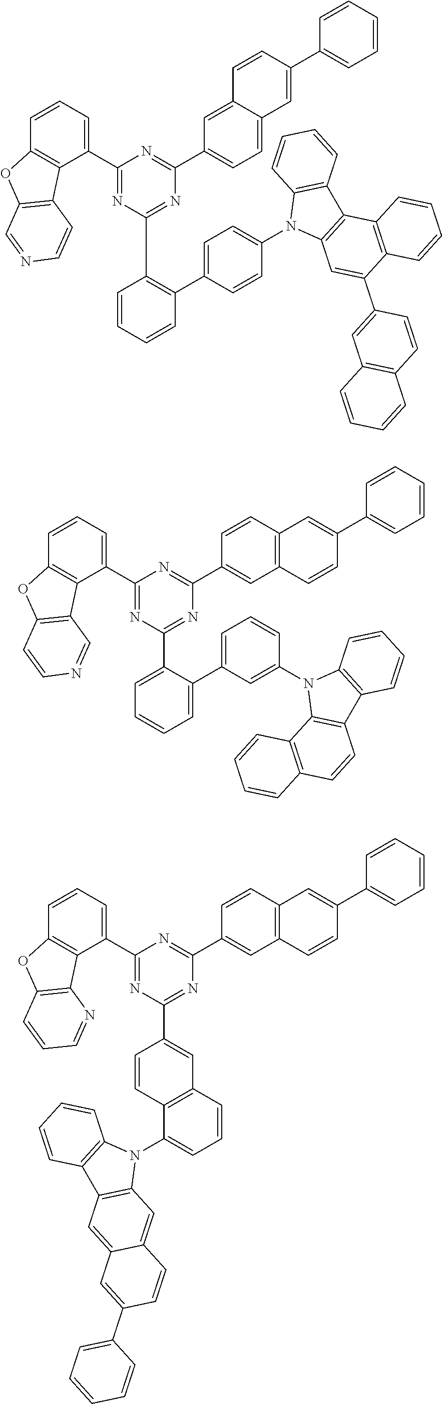

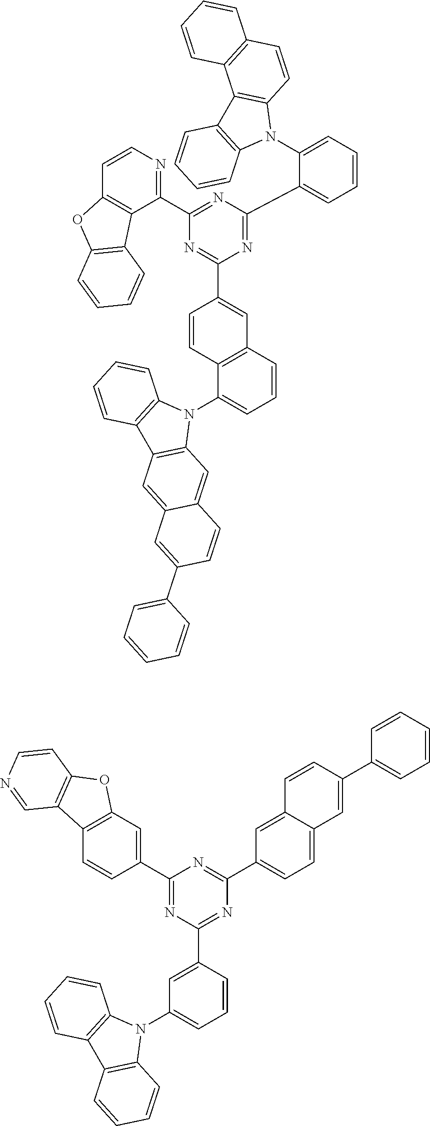 [SMILES: c1ccc(-c2ccc3cc4c(cc3c2)c2ccccc2n4-c2cccc3cc(-c4nc(-c5ccccc5-n5c6ccccc6c6c7ccccc7ccc65)nc(-c5nccc6oc7ccccc7c56)n4)ccc23)cc1 ; c1ccc(-c2ccc3cc(-c4nc(-c5cccc(-n6c7ccccc7c7ccccc76)c5)nc(-c5ccc6c(c5)oc5ccncc56)n4)ccc3c2)cc1]

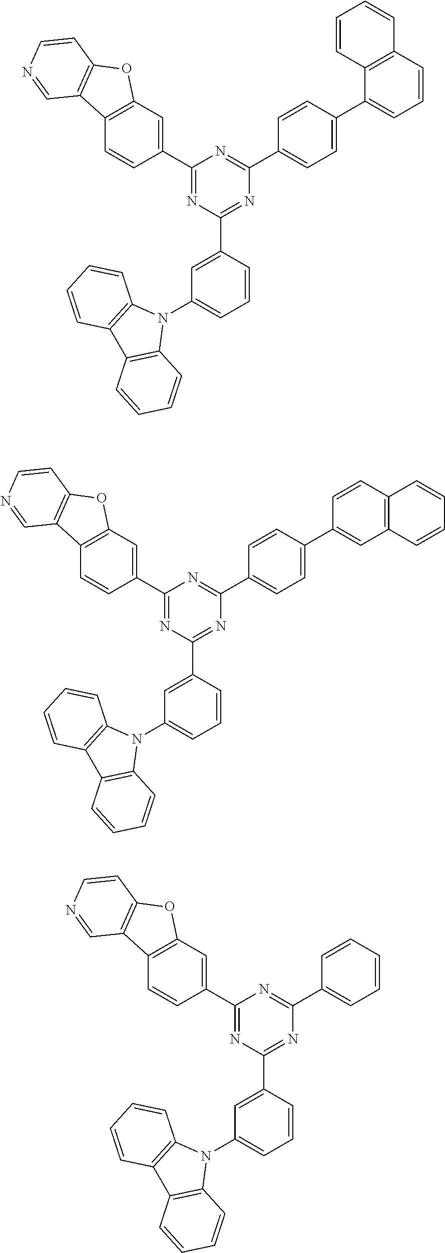

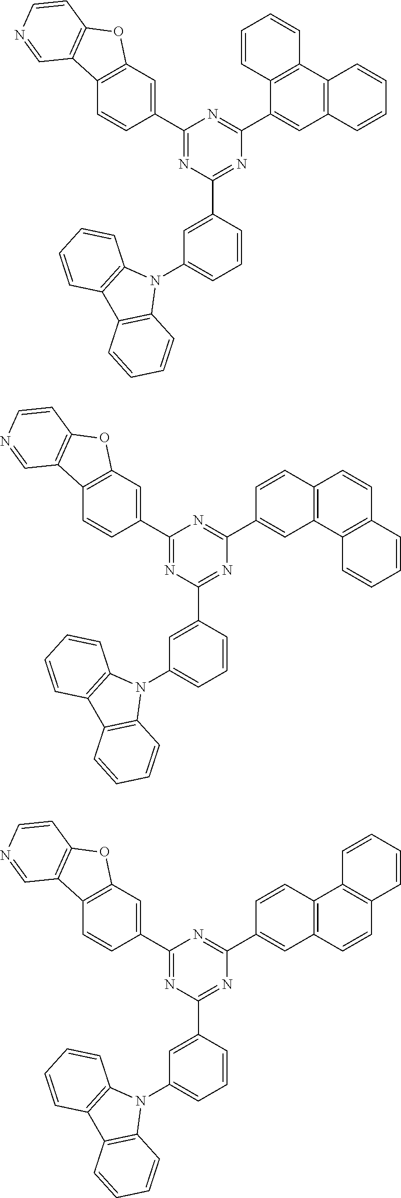 [SMILES: c1cc(-c2nc(-c3ccc4c(c3)oc3ccncc34)nc(-c3cc4ccccc4c4ccccc34)n2)cc(-n2c3ccccc3c3ccccc32)c1 ; c1cc(-c2nc(-c3ccc4c(c3)oc3ccncc34)nc(-c3ccc4ccc5ccccc5c4c3)n2)cc(-n2c3ccccc3c3ccccc32)c1 ; c1cc(-c2nc(-c3ccc4c(ccc5ccccc54)c3)nc(-c3ccc4c(c3)oc3ccncc34)n2)cc(-n2c3ccccc3c3ccccc32)c1]

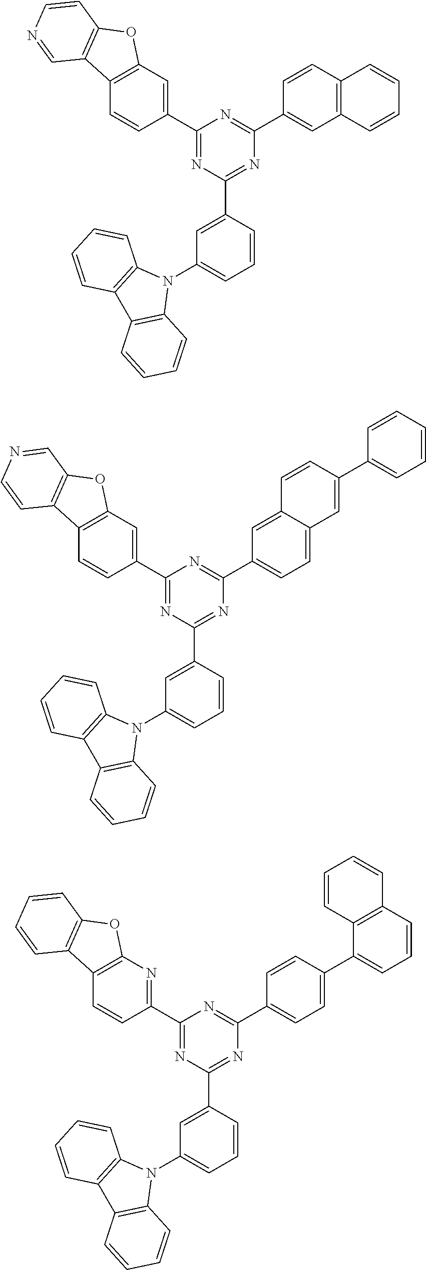 [SMILES: c1cc(-c2nc(-c3ccc4ccccc4c3)nc(-c3ccc4c(c3)oc3ccncc34)n2)cc(-n2c3ccccc3c3ccccc32)c1 ; c1ccc(-c2ccc3cc(-c4nc(-c5cccc(-n6c7ccccc7c7ccccc76)c5)nc(-c5ccc6c(c5)oc5cnccc56)n4)ccc3c2)cc1 ; c1cc(-c2nc(-c3ccc(-c4cccc5ccccc45)cc3)nc(-c3ccc4c(n3)oc3ccccc34)n2)cc(-n2c3ccccc3c3ccccc32)c1]

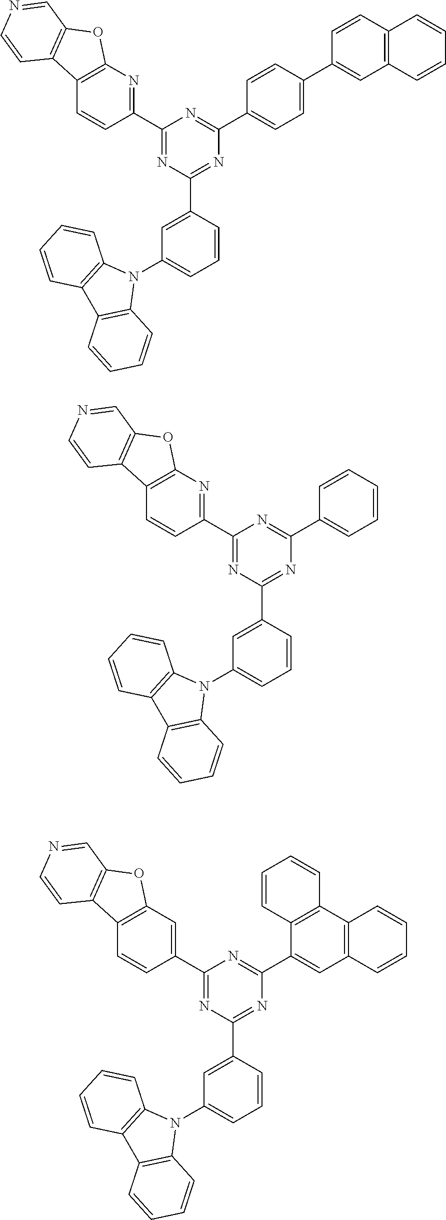

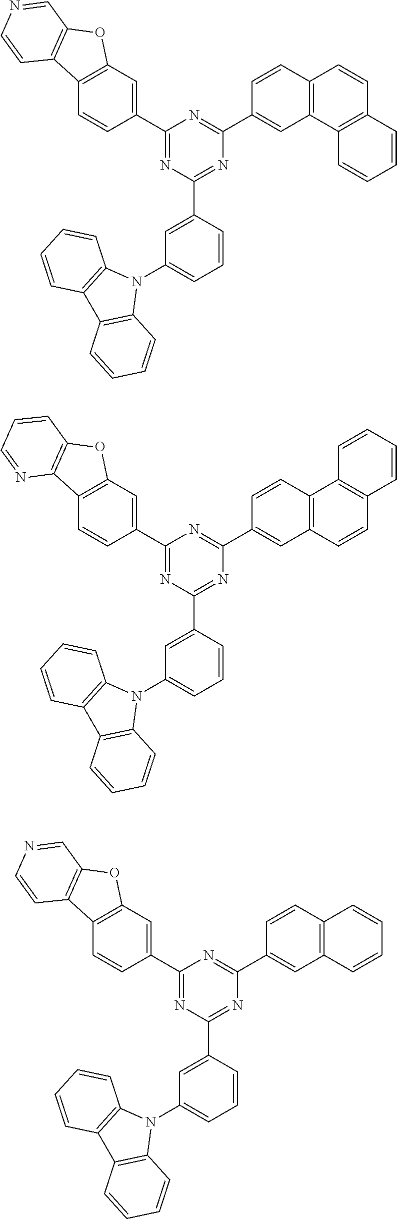 [SMILES: c1cc(-c2nc(-c3ccc4c(c3)oc3cnccc34)nc(-c3ccc4ccc5ccccc5c4c3)n2)cc(-n2c3ccccc3c3ccccc32)c1 ; c1cc(-c2nc(-c3ccc4c(ccc5ccccc54)c3)nc(-c3ccc4c(c3)oc3cccnc34)n2)cc(-n2c3ccccc3c3ccccc32)c1 ; c1cc(-c2nc(-c3ccc4ccccc4c3)nc(-c3ccc4c(c3)oc3cnccc34)n2)cc(-n2c3ccccc3c3ccccc32)c1]

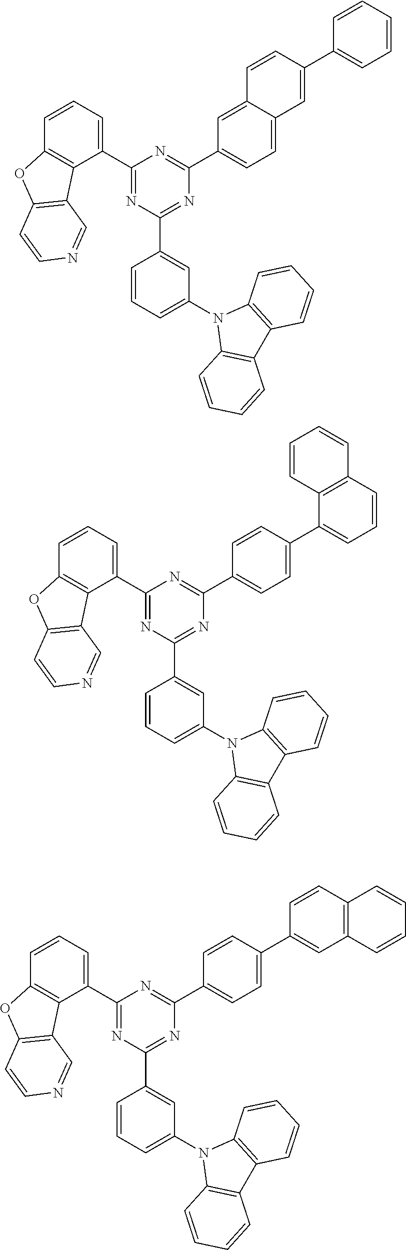 [SMILES: c1ccc(-c2ccc3cc(-c4nc(-c5cccc(-n6c7ccccc7c7ccccc76)c5)nc(-c5cccc6oc7ccncc7c56)n4)ccc3c2)cc1 ; c1cc(-c2nc(-c3ccc(-c4cccc5ccccc45)cc3)nc(-c3cccc4oc5ccncc5c34)n2)cc(-n2c3ccccc3c3ccccc32)c1 ; c1cc(-c2nc(-c3ccc(-c4ccc5ccccc5c4)cc3)nc(-c3cccc4oc5ccncc5c34)n2)cc(-n2c3ccccc3c3ccccc32)c1]

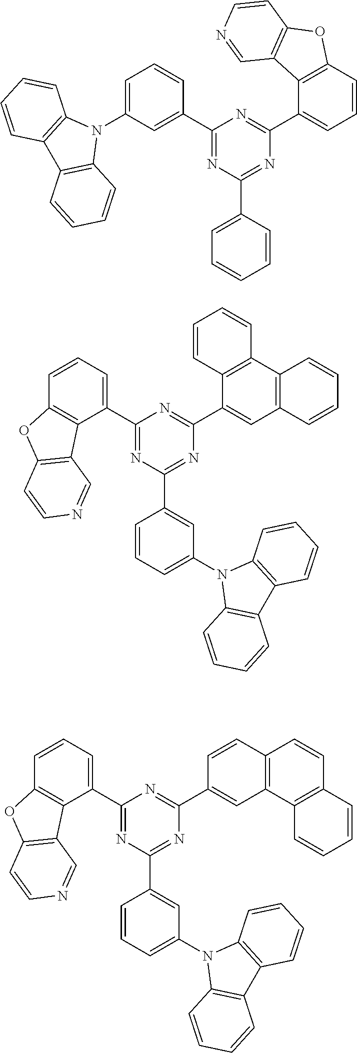

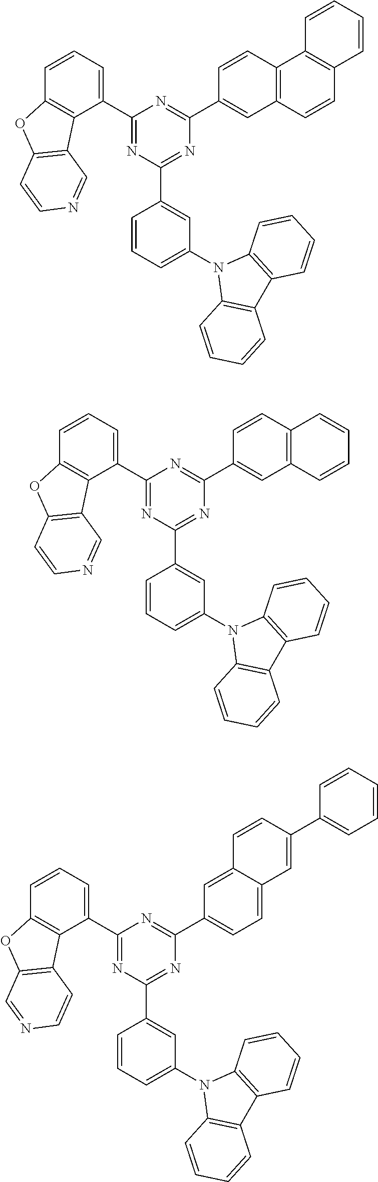 [SMILES: c1cc(-c2nc(-c3ccc4c(ccc5ccccc54)c3)nc(-c3cccc4oc5ccncc5c34)n2)cc(-n2c3ccccc3c3ccccc32)c1 ; c1cc(-c2nc(-c3ccc4ccccc4c3)nc(-c3cccc4oc5ccncc5c34)n2)cc(-n2c3ccccc3c3ccccc32)c1 ; c1ccc(-c2ccc3cc(-c4nc(-c5cccc(-n6c7ccccc7c7ccccc76)c5)nc(-c5cccc6oc7cnccc7c56)n4)ccc3c2)cc1]

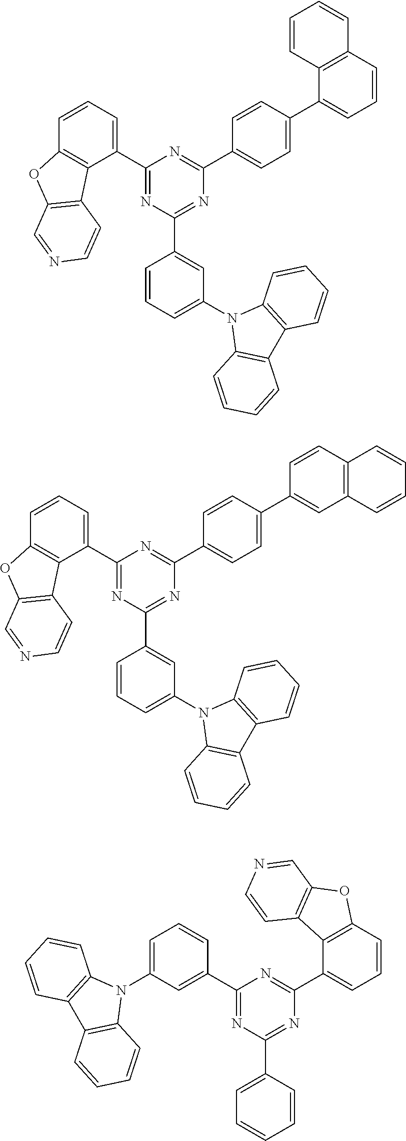 [SMILES: c1cc(-c2nc(-c3ccc(-c4cccc5ccccc45)cc3)nc(-c3cccc4oc5cnccc5c34)n2)cc(-n2c3ccccc3c3ccccc32)c1 ; c1cc(-c2nc(-c3ccc(-c4ccc5ccccc5c4)cc3)nc(-c3cccc4oc5cnccc5c34)n2)cc(-n2c3ccccc3c3ccccc32)c1 ; c1ccc(-c2nc(-c3cccc(-n4c5ccccc5c5ccccc54)c3)nc(-c3cccc4oc5cnccc5c34)n2)cc1]

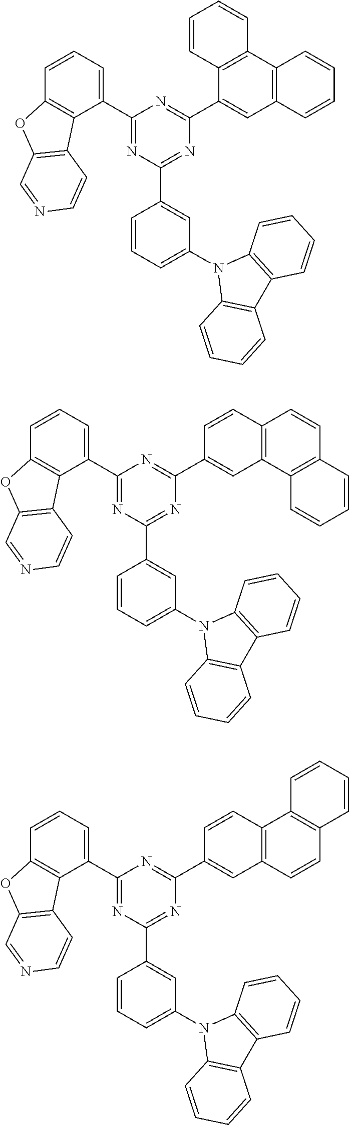

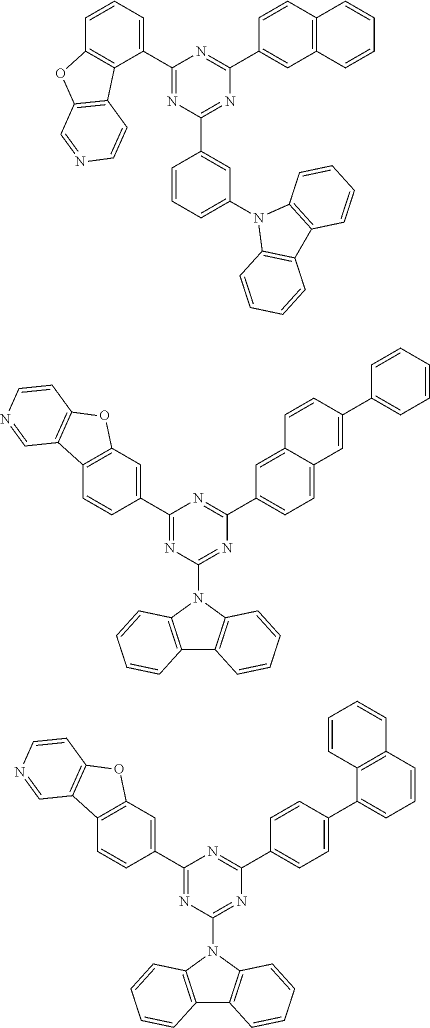 [SMILES: c1cc(-c2nc(-c3ccc4ccccc4c3)nc(-c3cccc4oc5cnccc5c34)n2)cc(-n2c3ccccc3c3ccccc32)c1 ; c1ccc(-c2ccc3cc(-c4nc(-c5ccc6c(c5)oc5ccncc56)nc(-n5c6ccccc6c6ccccc65)n4)ccc3c2)cc1 ; c1ccc2c(-c3ccc(-c4nc(-c5ccc6c(c5)oc5ccncc56)nc(-n5c6ccccc6c6ccccc65)n4)cc3)cccc2c1]

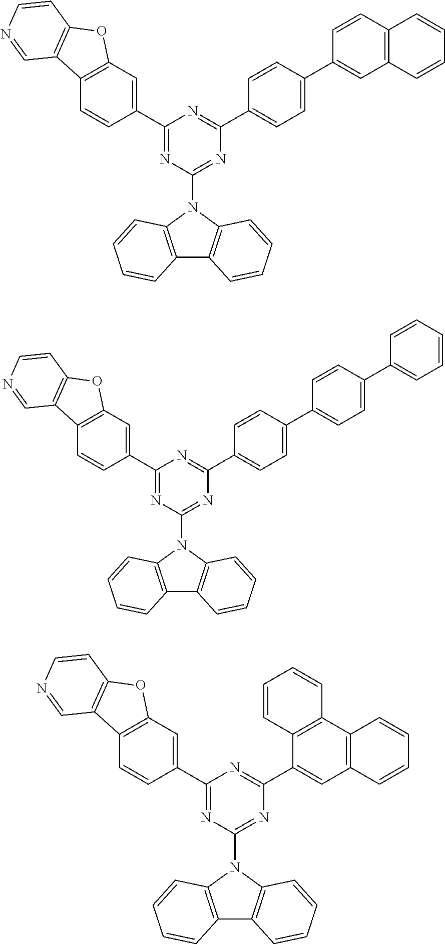 [SMILES: c1ccc2cc(-c3ccc(-c4nc(-c5ccc6c(c5)oc5ccncc56)nc(-n5c6ccccc6c6ccccc65)n4)cc3)ccc2c1 ; c1ccc(-c2ccc(-c3ccc(-c4nc(-c5ccc6c(c5)oc5ccncc56)nc(-n5c6ccccc6c6ccccc65)n4)cc3)cc2)cc1 ; c1ccc2c(c1)cc(-c1nc(-c3ccc4c(c3)oc3ccncc34)nc(-n3c4ccccc4c4ccccc43)n1)c1ccccc12]

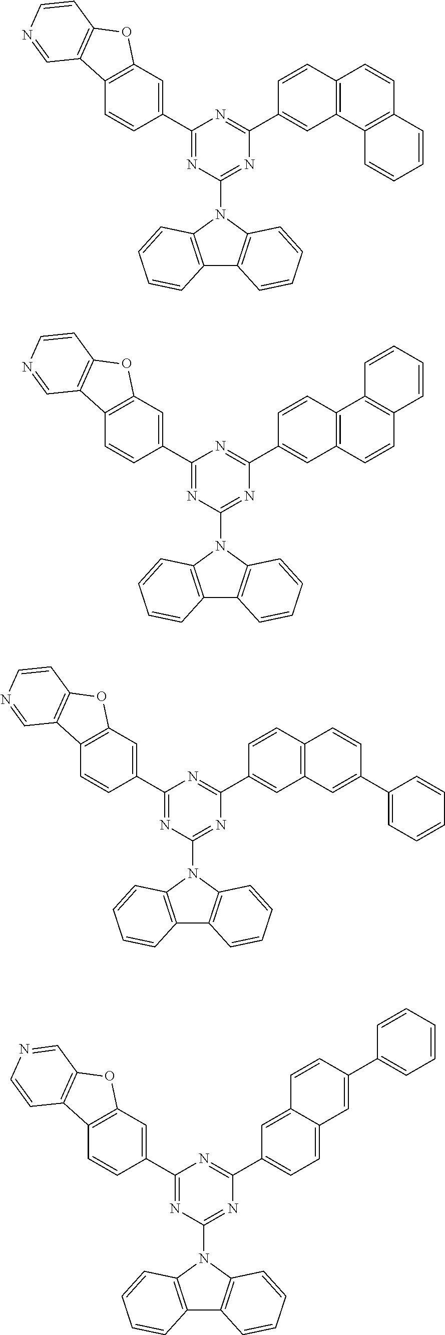 [SMILES: c1ccc2c(c1)ccc1ccc(-c3nc(-c4ccc5c(c4)oc4ccncc45)nc(-n4c5ccccc5c5ccccc54)n3)cc12 ; c1ccc2c(c1)ccc1cc(-c3nc(-c4ccc5c(c4)oc4ccncc45)nc(-n4c5ccccc5c5ccccc54)n3)ccc12 ; c1ccc(-c2ccc3ccc(-c4nc(-c5ccc6c(c5)oc5ccncc56)nc(-n5c6ccccc6c6ccccc65)n4)cc3c2)cc1 ; c1ccc(-c2ccc3cc(-c4nc(-c5ccc6c(c5)oc5cnccc56)nc(-n5c6ccccc6c6ccccc65)n4)ccc3c2)cc1]

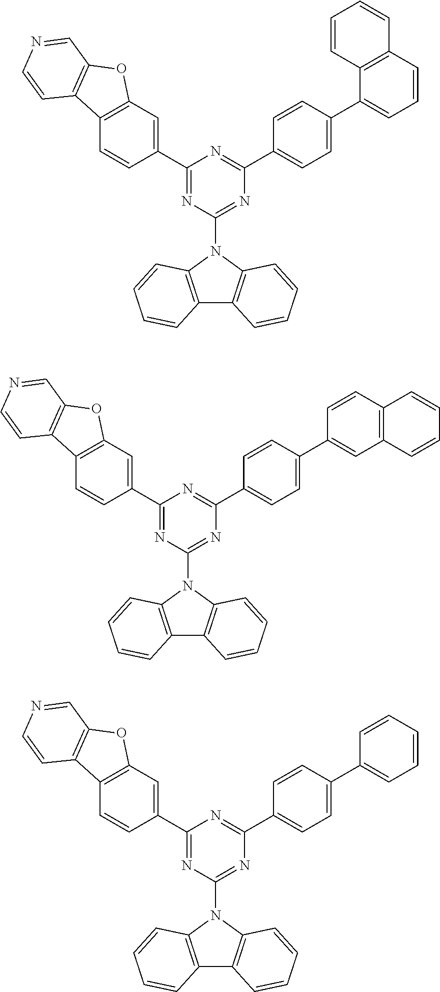 [SMILES: c1ccc2c(-c3ccc(-c4nc(-c5ccc6c(c5)oc5cnccc56)nc(-n5c6ccccc6c6ccccc65)n4)cc3)cccc2c1 ; c1ccc2cc(-c3ccc(-c4nc(-c5ccc6c(c5)oc5cnccc56)nc(-n5c6ccccc6c6ccccc65)n4)cc3)ccc2c1 ; c1ccc(-c2ccc(-c3nc(-c4ccc5c(c4)oc4cnccc45)nc(-n4c5ccccc5c5ccccc54)n3)cc2)cc1]

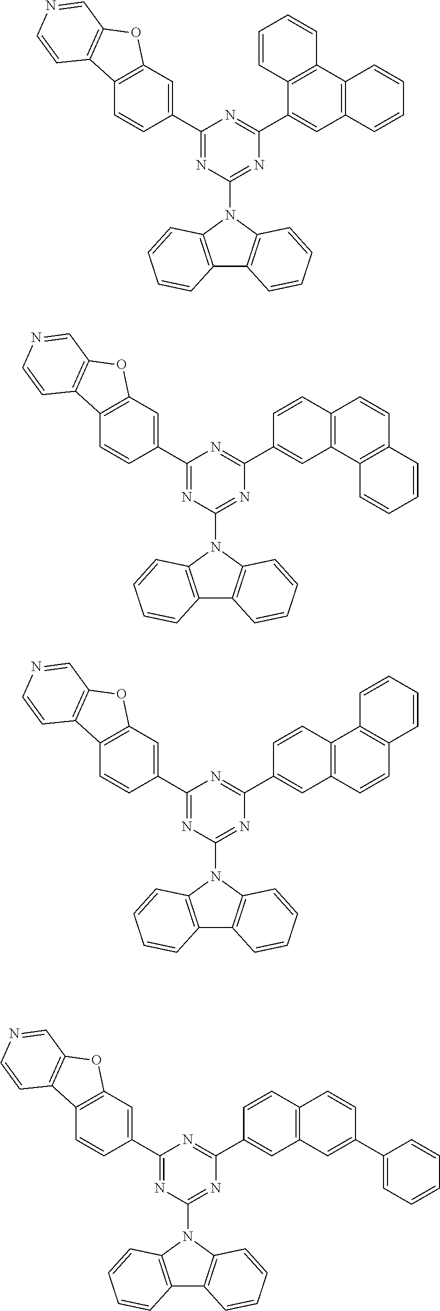 [SMILES: c1ccc2c(c1)cc(-c1nc(-c3ccc4c(c3)oc3cnccc34)nc(-n3c4ccccc4c4ccccc43)n1)c1ccccc12 ; c1ccc2c(c1)ccc1ccc(-c3nc(-c4ccc5c(c4)oc4cnccc45)nc(-n4c5ccccc5c5ccccc54)n3)cc12 ; c1ccc2c(c1)ccc1cc(-c3nc(-c4ccc5c(c4)oc4cnccc45)nc(-n4c5ccccc5c5ccccc54)n3)ccc12 ; c1ccc(-c2ccc3ccc(-c4nc(-c5ccc6c(c5)oc5cnccc56)nc(-n5c6ccccc6c6ccccc65)n4)cc3c2)cc1]

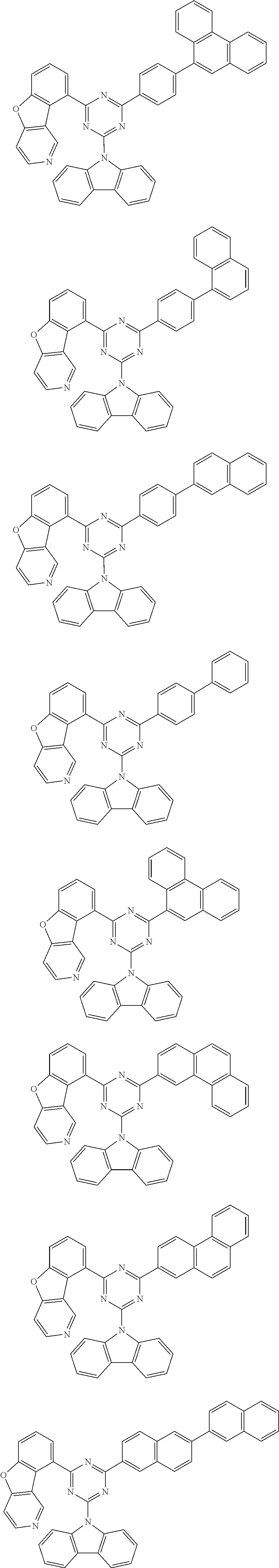

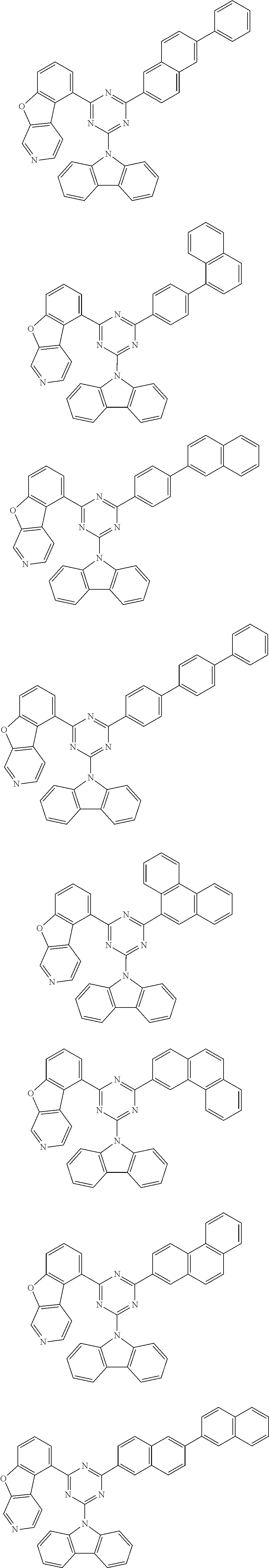

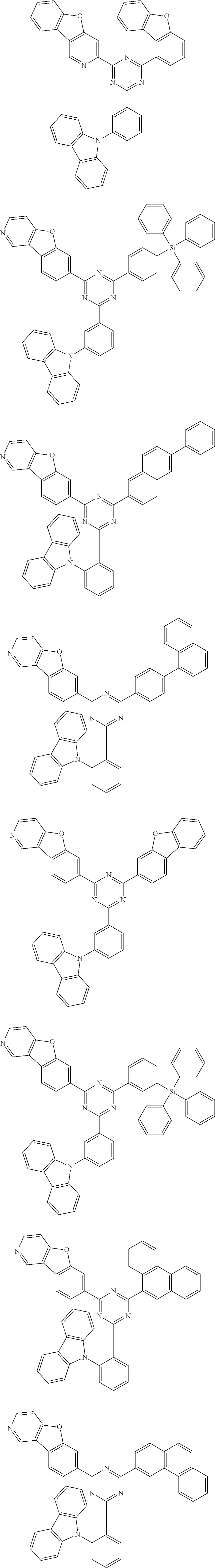

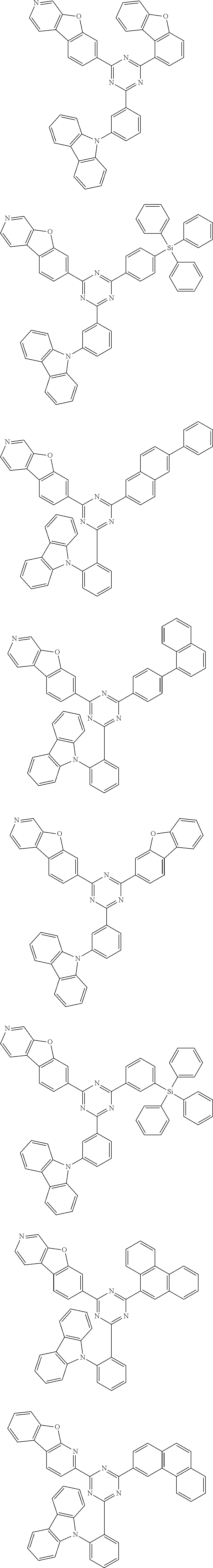

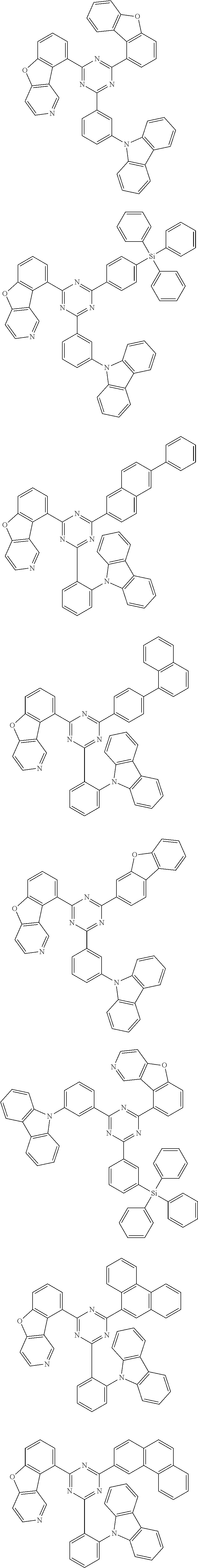

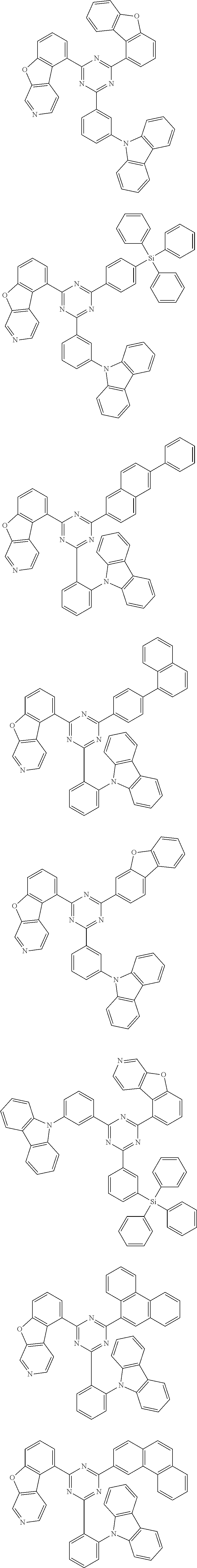

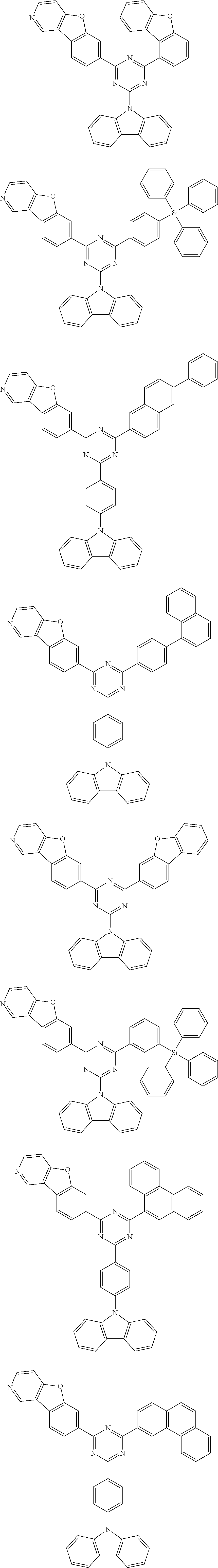

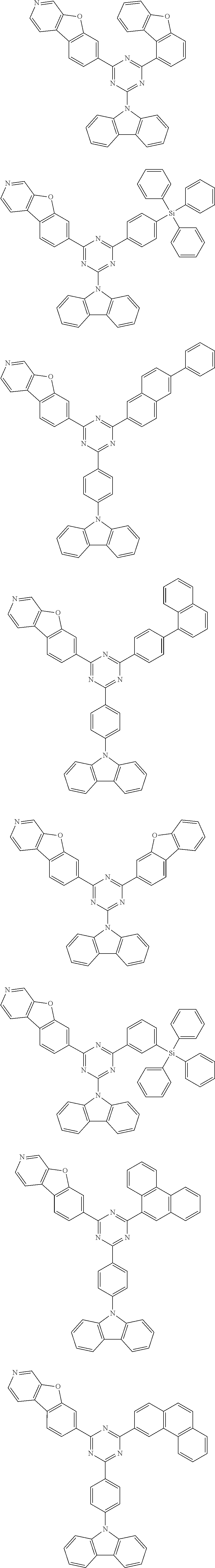

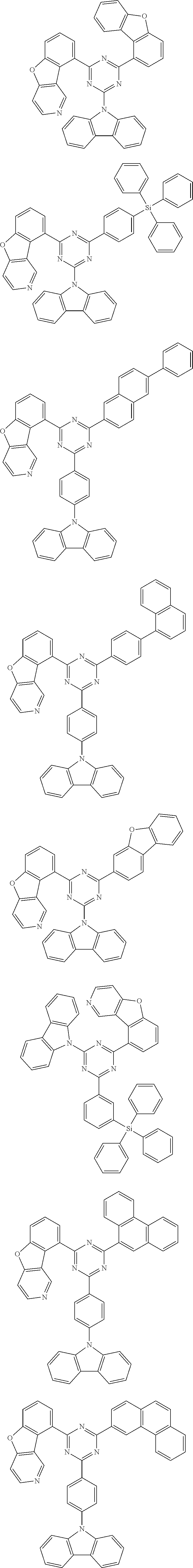

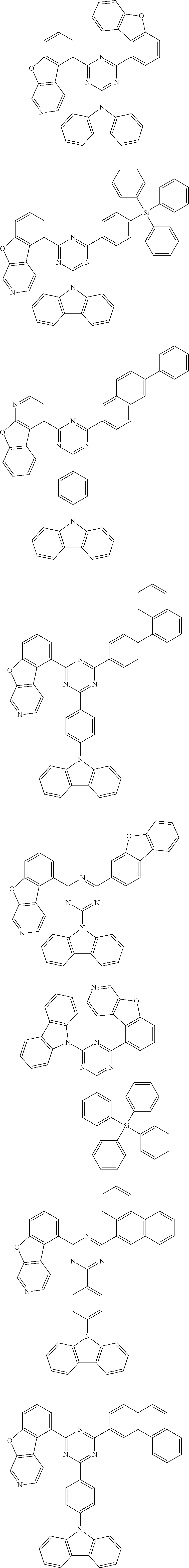

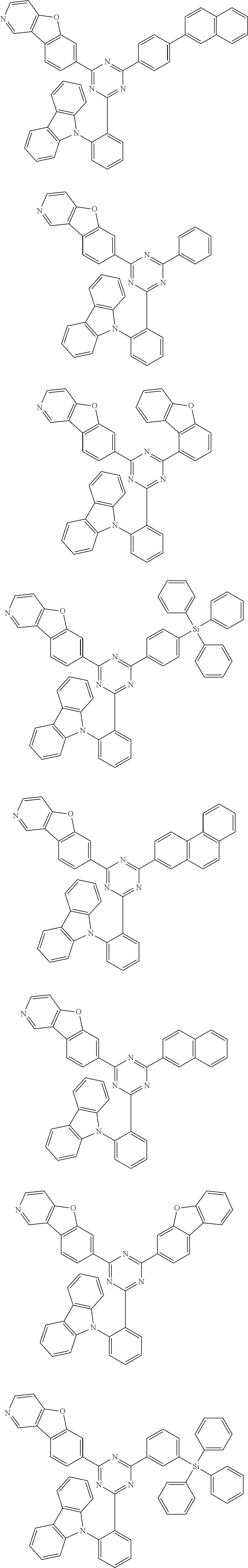

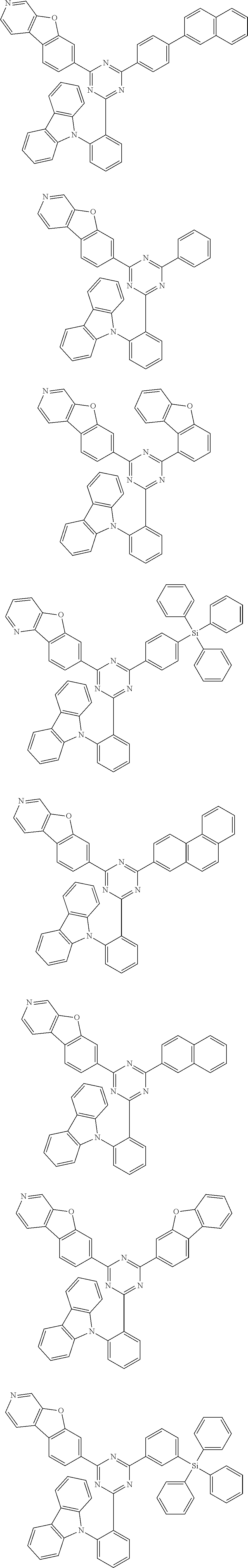

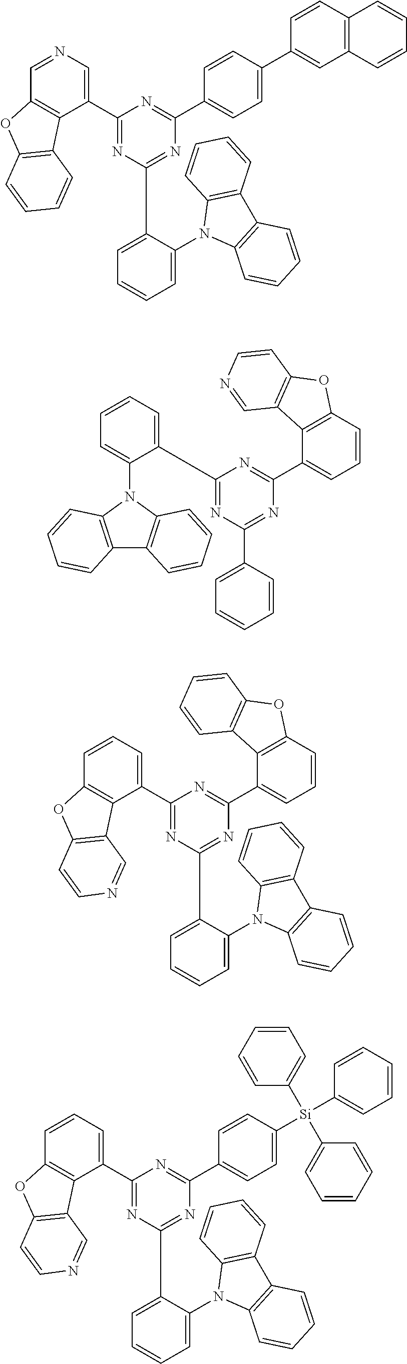 [SMILES: c1ccc(-n2c3ccccc3c3ccccc32)c(-c2nc(-c3ccc(-c4ccc5ccccc5c4)cc3)nc(-c3cncc4oc5ccccc5c34)n2)c1 ; c1ccc(-c2nc(-c3ccccc3-n3c4ccccc4c4ccccc43)nc(-c3cccc4oc5ccncc5c34)n2)cc1 ; c1ccc(-n2c3ccccc3c3ccccc32)c(-c2nc(-c3cccc4oc5ccccc5c34)nc(-c3cccc4oc5ccncc5c34)n2)c1 ; c1ccc([Si](c2ccccc2)(c2ccccc2)c2ccc(-c3nc(-c4ccccc4-n4c5ccccc5c5ccccc54)nc(-c4cccc5oc6ccncc6c45)n3)cc2)cc1]

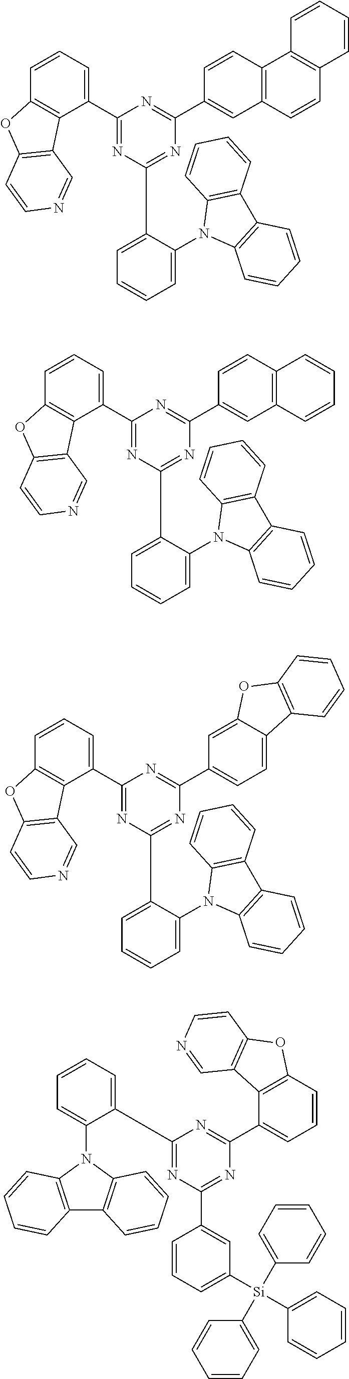 [SMILES: c1ccc(-n2c3ccccc3c3ccccc32)c(-c2nc(-c3ccc4c(ccc5ccccc54)c3)nc(-c3cccc4oc5ccncc5c34)n2)c1 ; c1ccc(-n2c3ccccc3c3ccccc32)c(-c2nc(-c3ccc4ccccc4c3)nc(-c3cccc4oc5ccncc5c34)n2)c1 ; c1ccc(-n2c3ccccc3c3ccccc32)c(-c2nc(-c3ccc4c(c3)oc3ccccc34)nc(-c3cccc4oc5ccncc5c34)n2)c1 ; c1ccc([Si](c2ccccc2)(c2ccccc2)c2cccc(-c3nc(-c4ccccc4-n4c5ccccc5c5ccccc54)nc(-c4cccc5oc6ccncc6c45)n3)c2)cc1]

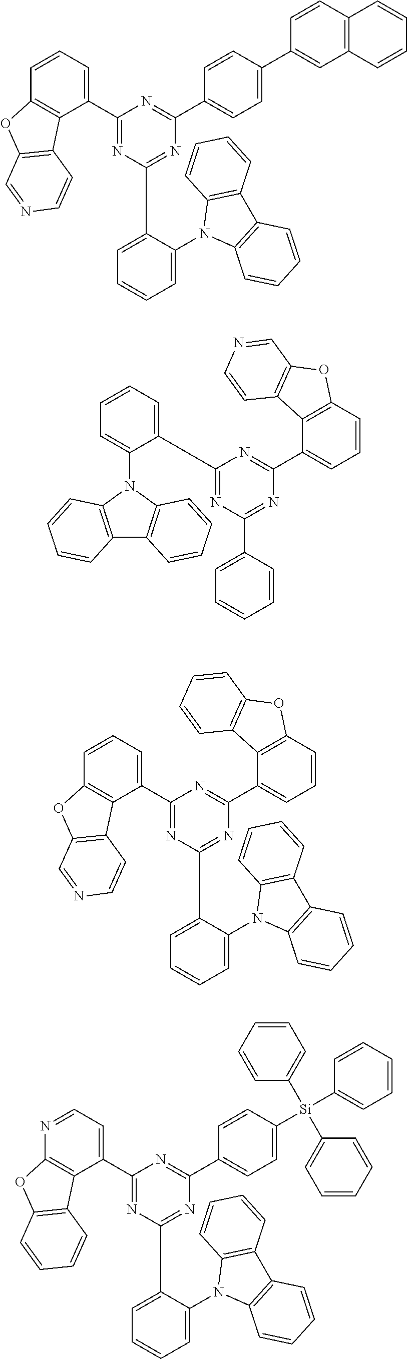 [SMILES: c1ccc(-n2c3ccccc3c3ccccc32)c(-c2nc(-c3ccc(-c4ccc5ccccc5c4)cc3)nc(-c3cccc4oc5cnccc5c34)n2)c1 ; c1ccc(-c2nc(-c3ccccc3-n3c4ccccc4c4ccccc43)nc(-c3cccc4oc5cnccc5c34)n2)cc1 ; c1ccc(-n2c3ccccc3c3ccccc32)c(-c2nc(-c3cccc4oc5ccccc5c34)nc(-c3cccc4oc5cnccc5c34)n2)c1 ; c1ccc([Si](c2ccccc2)(c2ccccc2)c2ccc(-c3nc(-c4ccccc4-n4c5ccccc5c5ccccc54)nc(-c4ccnc5oc6ccccc6c45)n3)cc2)cc1]

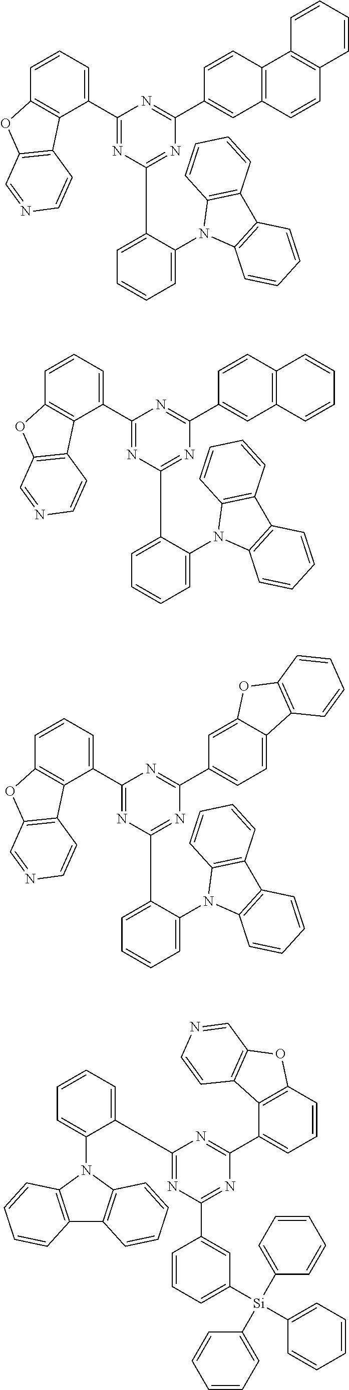 [SMILES: c1ccc(-n2c3ccccc3c3ccccc32)c(-c2nc(-c3ccc4c(ccc5ccccc54)c3)nc(-c3cccc4oc5cnccc5c34)n2)c1 ; c1ccc(-n2c3ccccc3c3ccccc32)c(-c2nc(-c3ccc4ccccc4c3)nc(-c3cccc4oc5cnccc5c34)n2)c1 ; c1ccc(-n2c3ccccc3c3ccccc32)c(-c2nc(-c3ccc4c(c3)oc3ccccc34)nc(-c3cccc4oc5cnccc5c34)n2)c1 ; c1ccc([Si](c2ccccc2)(c2ccccc2)c2cccc(-c3nc(-c4ccccc4-n4c5ccccc5c5ccccc54)nc(-c4cccc5oc6cnccc6c45)n3)c2)cc1]

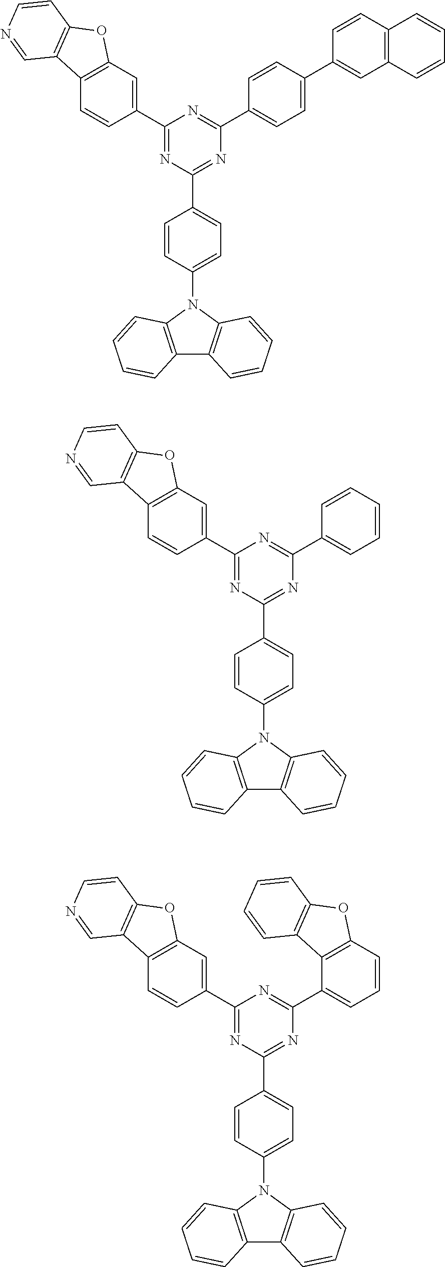 [SMILES: c1ccc2cc(-c3ccc(-c4nc(-c5ccc(-n6c7ccccc7c7ccccc76)cc5)nc(-c5ccc6c(c5)oc5ccncc56)n4)cc3)ccc2c1 ; c1ccc(-c2nc(-c3ccc(-n4c5ccccc5c5ccccc54)cc3)nc(-c3ccc4c(c3)oc3ccncc34)n2)cc1 ; c1ccc2c(c1)oc1cccc(-c3nc(-c4ccc(-n5c6ccccc6c6ccccc65)cc4)nc(-c4ccc5c(c4)oc4ccncc45)n3)c12]

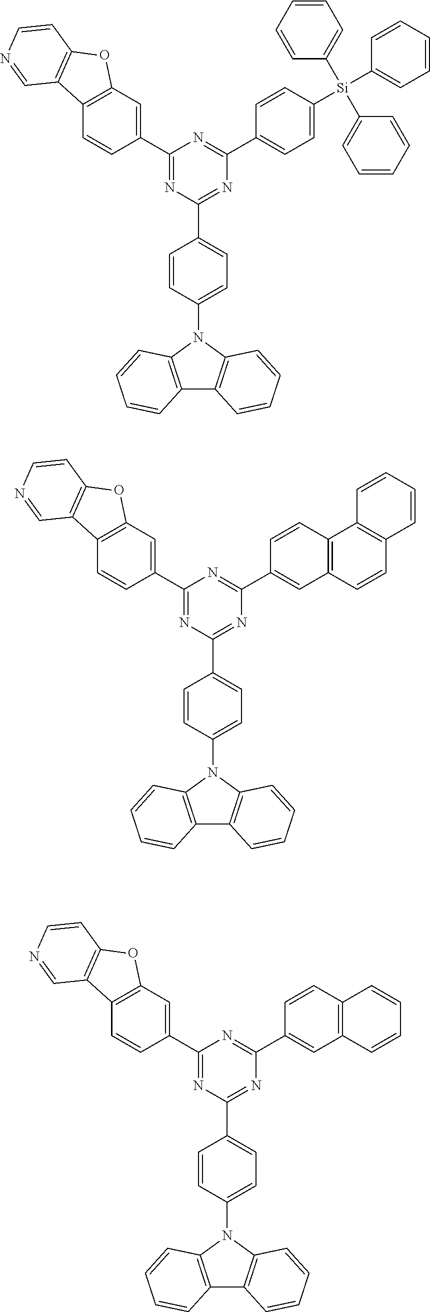 [SMILES: c1ccc([Si](c2ccccc2)(c2ccccc2)c2ccc(-c3nc(-c4ccc(-n5c6ccccc6c6ccccc65)cc4)nc(-c4ccc5c(c4)oc4ccncc45)n3)cc2)cc1 ; c1ccc2c(c1)ccc1cc(-c3nc(-c4ccc(-n5c6ccccc6c6ccccc65)cc4)nc(-c4ccc5c(c4)oc4ccncc45)n3)ccc12 ; c1ccc2cc(-c3nc(-c4ccc(-n5c6ccccc6c6ccccc65)cc4)nc(-c4ccc5c(c4)oc4ccncc45)n3)ccc2c1]

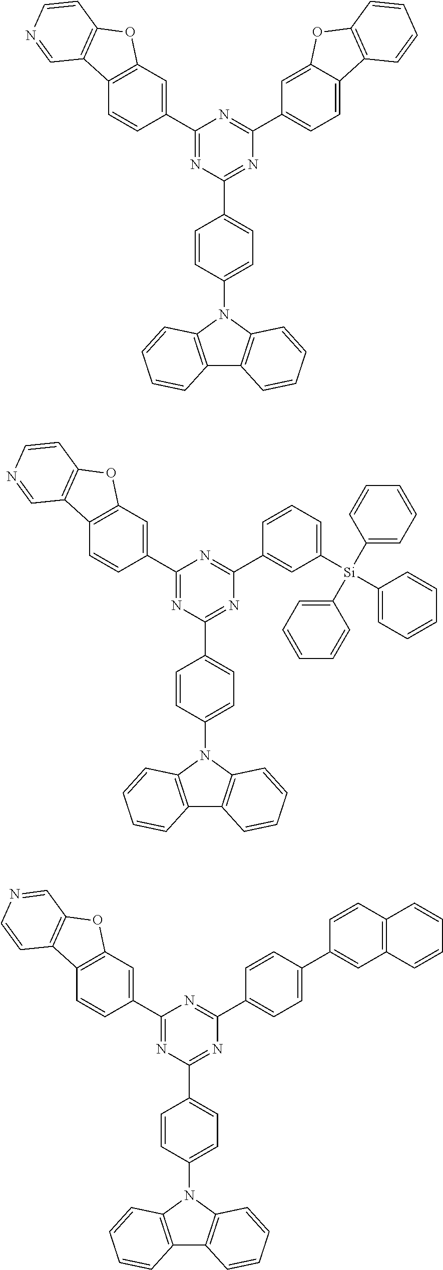 [SMILES: c1ccc2c(c1)oc1cc(-c3nc(-c4ccc(-n5c6ccccc6c6ccccc65)cc4)nc(-c4ccc5c(c4)oc4ccncc45)n3)ccc12 ; c1ccc([Si](c2ccccc2)(c2ccccc2)c2cccc(-c3nc(-c4ccc(-n5c6ccccc6c6ccccc65)cc4)nc(-c4ccc5c(c4)oc4ccncc45)n3)c2)cc1 ; c1ccc2cc(-c3ccc(-c4nc(-c5ccc(-n6c7ccccc7c7ccccc76)cc5)nc(-c5ccc6c(c5)oc5cnccc56)n4)cc3)ccc2c1]

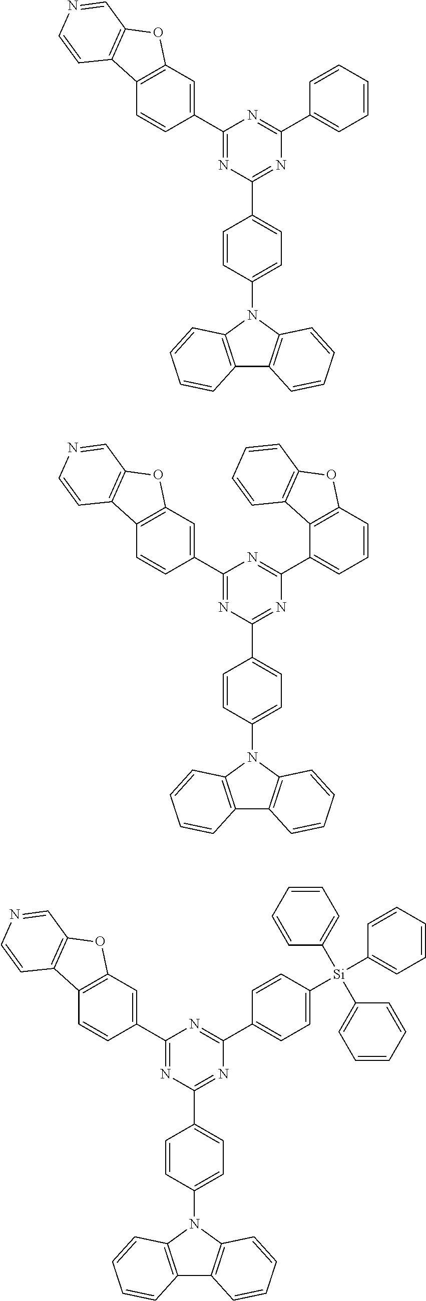 [SMILES: c1ccc(-c2nc(-c3ccc(-n4c5ccccc5c5ccccc54)cc3)nc(-c3ccc4c(c3)oc3cnccc34)n2)cc1 ; c1ccc2c(c1)oc1cccc(-c3nc(-c4ccc(-n5c6ccccc6c6ccccc65)cc4)nc(-c4ccc5c(c4)oc4cnccc45)n3)c12 ; c1ccc([Si](c2ccccc2)(c2ccccc2)c2ccc(-c3nc(-c4ccc(-n5c6ccccc6c6ccccc65)cc4)nc(-c4ccc5c(c4)oc4cnccc45)n3)cc2)cc1]

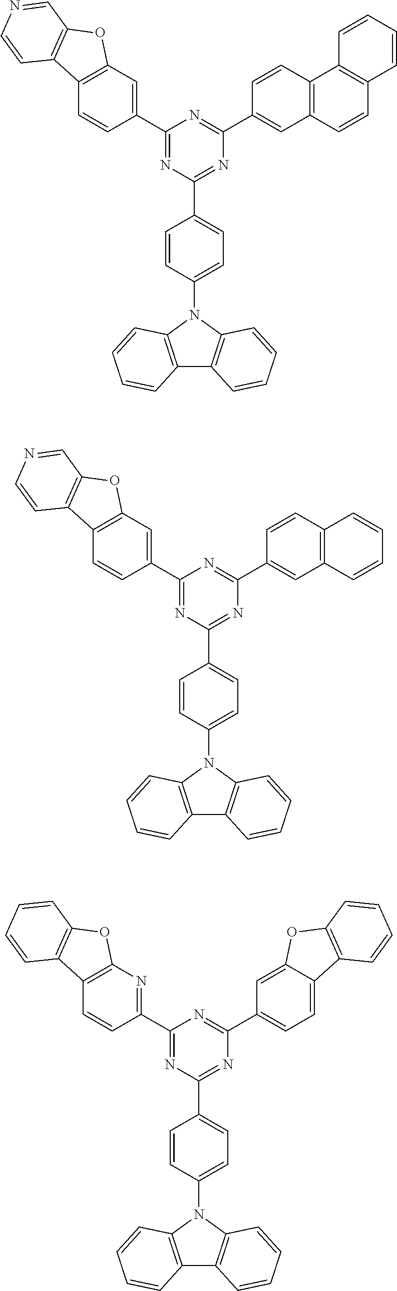 [SMILES: c1ccc2c(c1)ccc1cc(-c3nc(-c4ccc(-n5c6ccccc6c6ccccc65)cc4)nc(-c4ccc5c(c4)oc4cnccc45)n3)ccc12 ; c1ccc2cc(-c3nc(-c4ccc(-n5c6ccccc6c6ccccc65)cc4)nc(-c4ccc5c(c4)oc4cnccc45)n3)ccc2c1 ; c1ccc2c(c1)oc1cc(-c3nc(-c4ccc(-n5c6ccccc6c6ccccc65)cc4)nc(-c4ccc5c(n4)oc4ccccc45)n3)ccc12]

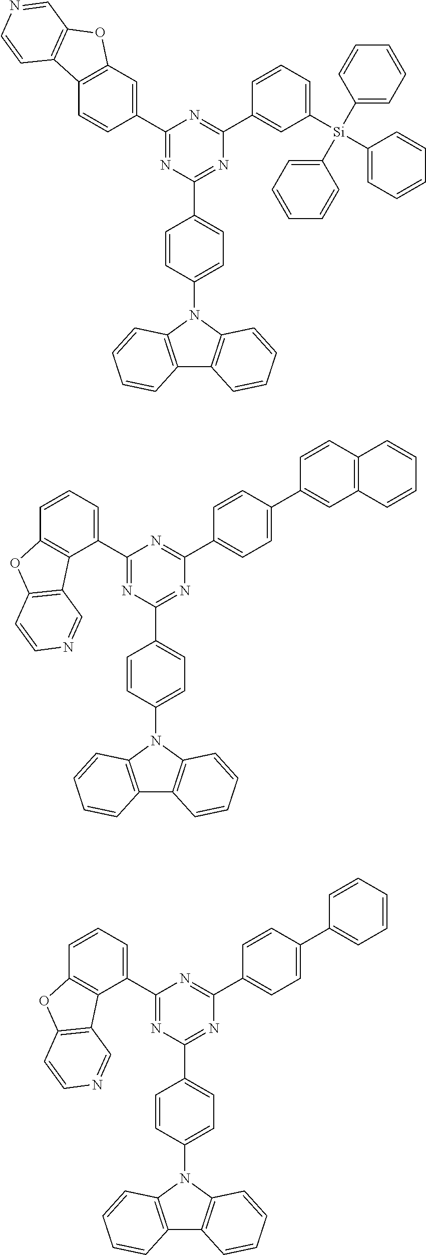 [SMILES: c1ccc([Si](c2ccccc2)(c2ccccc2)c2cccc(-c3nc(-c4ccc(-n5c6ccccc6c6ccccc65)cc4)nc(-c4ccc5c(c4)oc4cnccc45)n3)c2)cc1 ; c1ccc2cc(-c3ccc(-c4nc(-c5ccc(-n6c7ccccc7c7ccccc76)cc5)nc(-c5cccc6oc7ccncc7c56)n4)cc3)ccc2c1 ; c1ccc(-c2ccc(-c3nc(-c4ccc(-n5c6ccccc6c6ccccc65)cc4)nc(-c4cccc5oc6ccncc6c45)n3)cc2)cc1]

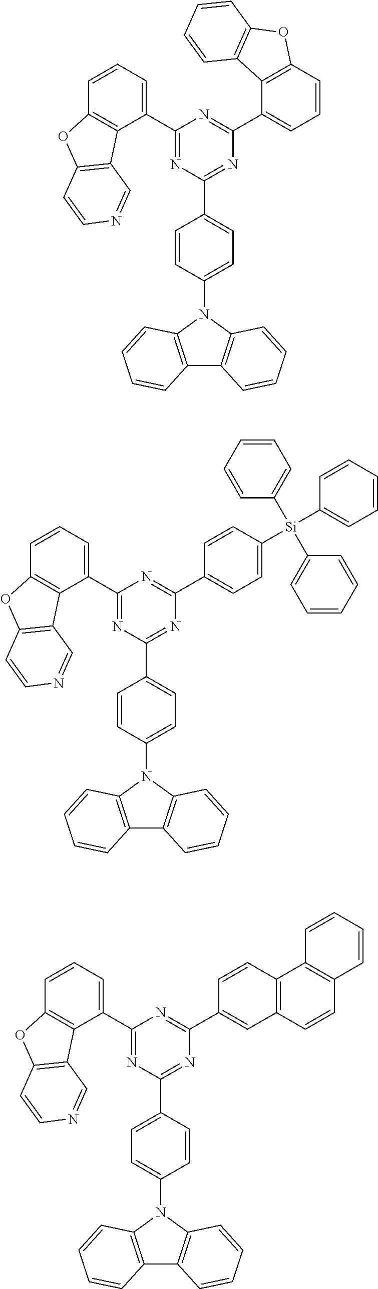 [SMILES: c1ccc2c(c1)oc1cccc(-c3nc(-c4ccc(-n5c6ccccc6c6ccccc65)cc4)nc(-c4cccc5oc6ccncc6c45)n3)c12 ; c1ccc([Si](c2ccccc2)(c2ccccc2)c2ccc(-c3nc(-c4ccc(-n5c6ccccc6c6ccccc65)cc4)nc(-c4cccc5oc6ccncc6c45)n3)cc2)cc1 ; c1ccc2c(c1)ccc1cc(-c3nc(-c4ccc(-n5c6ccccc6c6ccccc65)cc4)nc(-c4cccc5oc6ccncc6c45)n3)ccc12]

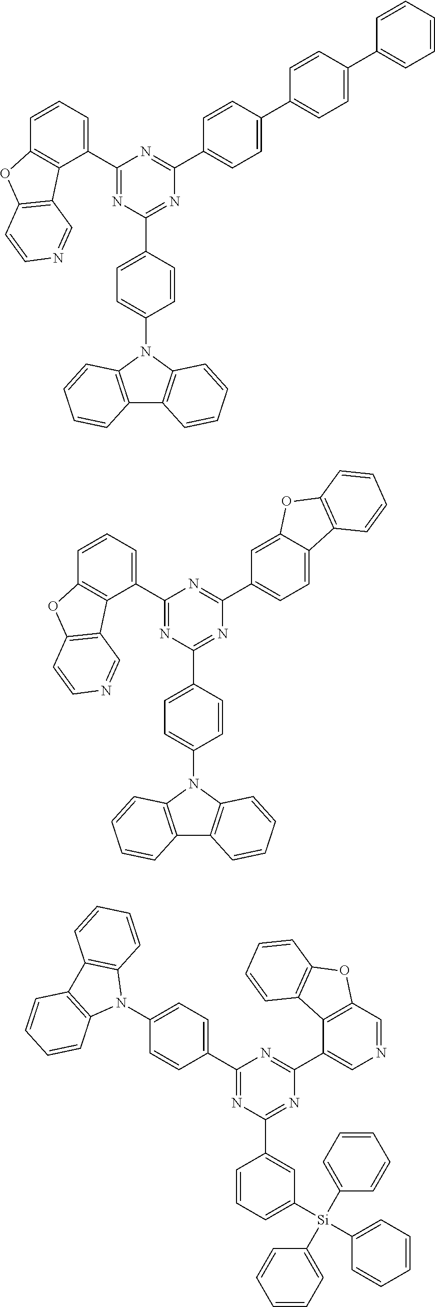 [SMILES: c1ccc(-c2ccc(-c3ccc(-c4nc(-c5ccc(-n6c7ccccc7c7ccccc76)cc5)nc(-c5cccc6oc7ccncc7c56)n4)cc3)cc2)cc1 ; c1ccc2c(c1)oc1cc(-c3nc(-c4ccc(-n5c6ccccc6c6ccccc65)cc4)nc(-c4cccc5oc6ccncc6c45)n3)ccc12 ; c1ccc([Si](c2ccccc2)(c2ccccc2)c2cccc(-c3nc(-c4ccc(-n5c6ccccc6c6ccccc65)cc4)nc(-c4cncc5oc6ccccc6c45)n3)c2)cc1]

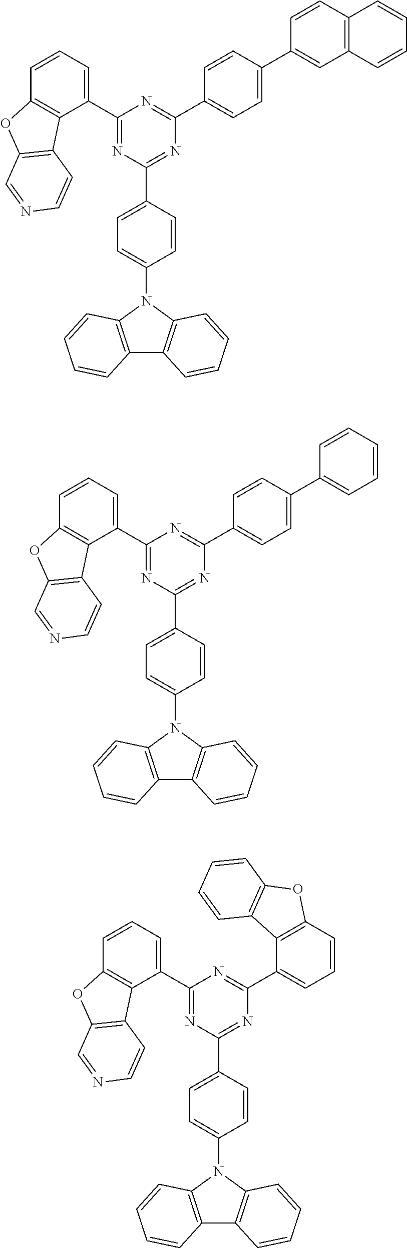 [SMILES: c1ccc2cc(-c3ccc(-c4nc(-c5ccc(-n6c7ccccc7c7ccccc76)cc5)nc(-c5cccc6oc7cnccc7c56)n4)cc3)ccc2c1 ; c1ccc(-c2ccc(-c3nc(-c4ccc(-n5c6ccccc6c6ccccc65)cc4)nc(-c4cccc5oc6cnccc6c45)n3)cc2)cc1 ; c1ccc2c(c1)oc1cccc(-c3nc(-c4ccc(-n5c6ccccc6c6ccccc65)cc4)nc(-c4cccc5oc6cnccc6c45)n3)c12]

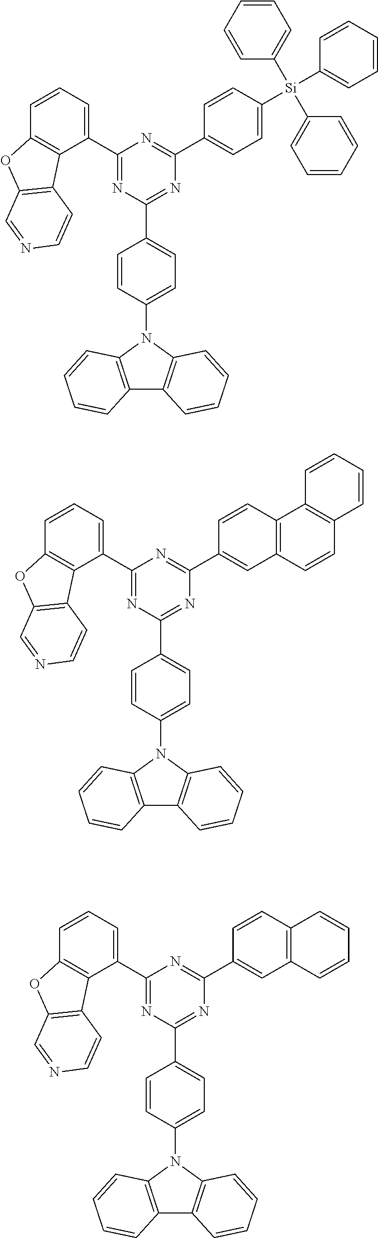 [SMILES: c1ccc([Si](c2ccccc2)(c2ccccc2)c2ccc(-c3nc(-c4ccc(-n5c6ccccc6c6ccccc65)cc4)nc(-c4cccc5oc6cnccc6c45)n3)cc2)cc1 ; c1ccc2c(c1)ccc1cc(-c3nc(-c4ccc(-n5c6ccccc6c6ccccc65)cc4)nc(-c4cccc5oc6cnccc6c45)n3)ccc12 ; c1ccc2cc(-c3nc(-c4ccc(-n5c6ccccc6c6ccccc65)cc4)nc(-c4cccc5oc6cnccc6c45)n3)ccc2c1]

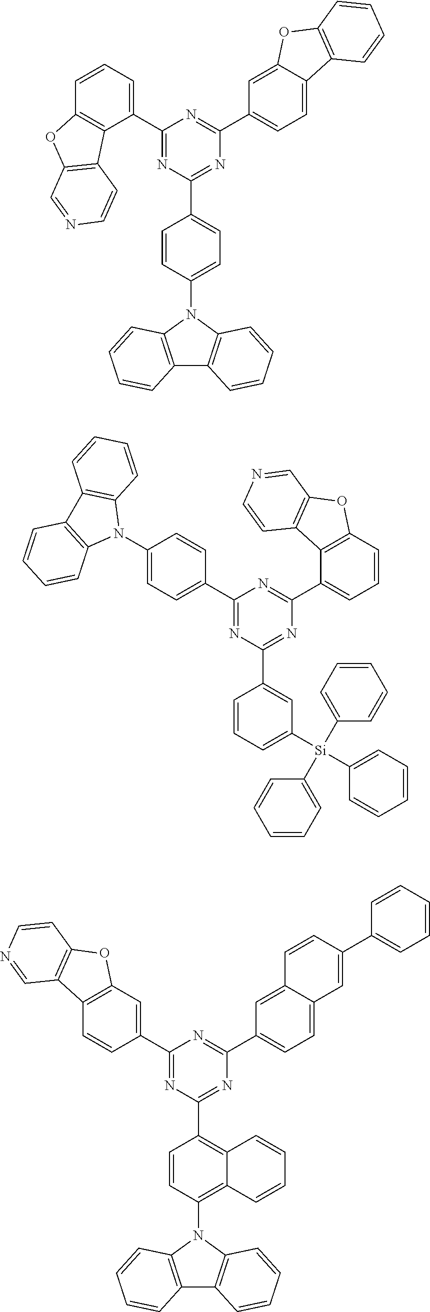 [SMILES: c1ccc2c(c1)oc1cc(-c3nc(-c4ccc(-n5c6ccccc6c6ccccc65)cc4)nc(-c4cccc5oc6cnccc6c45)n3)ccc12 ; c1ccc([Si](c2ccccc2)(c2ccccc2)c2cccc(-c3nc(-c4ccc(-n5c6ccccc6c6ccccc65)cc4)nc(-c4cccc5oc6cnccc6c45)n3)c2)cc1 ; c1ccc(-c2ccc3cc(-c4nc(-c5ccc6c(c5)oc5ccncc56)nc(-c5ccc(-n6c7ccccc7c7ccccc76)c6ccccc56)n4)ccc3c2)cc1]

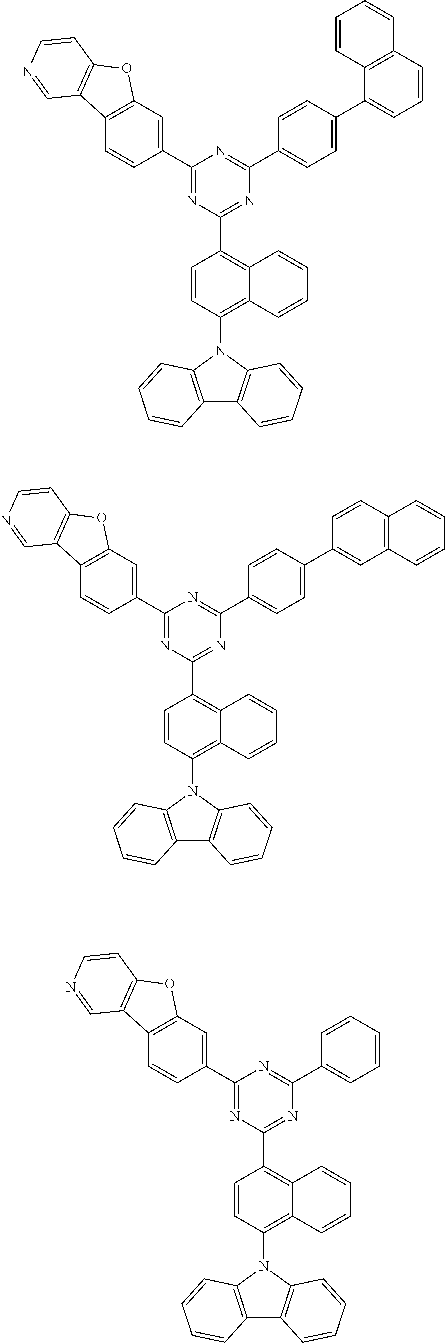 [SMILES: c1ccc2c(-c3ccc(-c4nc(-c5ccc6c(c5)oc5ccncc56)nc(-c5ccc(-n6c7ccccc7c7ccccc76)c6ccccc56)n4)cc3)cccc2c1 ; c1ccc2cc(-c3ccc(-c4nc(-c5ccc6c(c5)oc5ccncc56)nc(-c5ccc(-n6c7ccccc7c7ccccc76)c6ccccc56)n4)cc3)ccc2c1 ; c1ccc(-c2nc(-c3ccc4c(c3)oc3ccncc34)nc(-c3ccc(-n4c5ccccc5c5ccccc54)c4ccccc34)n2)cc1]

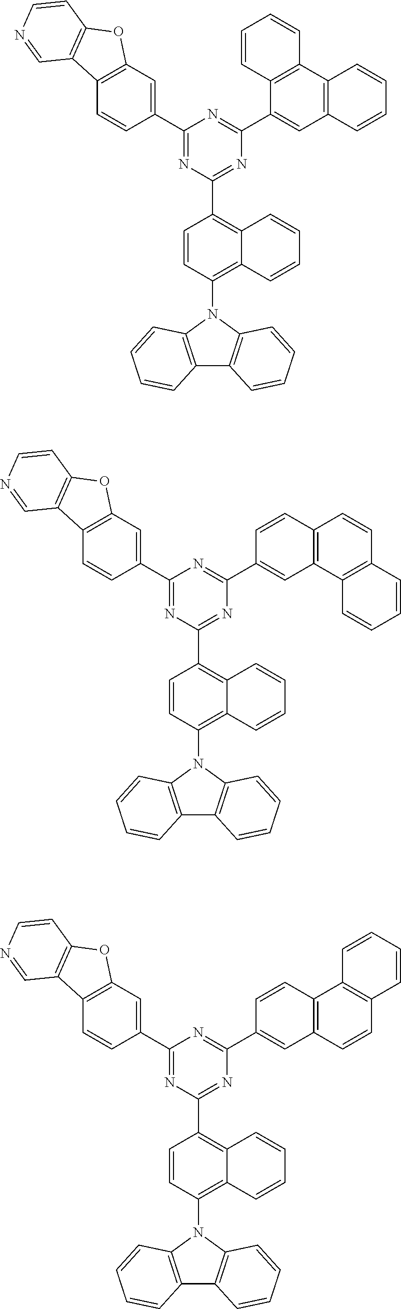 [SMILES: c1ccc2c(c1)cc(-c1nc(-c3ccc4c(c3)oc3ccncc34)nc(-c3ccc(-n4c5ccccc5c5ccccc54)c4ccccc34)n1)c1ccccc12 ; c1ccc2c(c1)ccc1ccc(-c3nc(-c4ccc5c(c4)oc4ccncc45)nc(-c4ccc(-n5c6ccccc6c6ccccc65)c5ccccc45)n3)cc12 ; c1ccc2c(c1)ccc1cc(-c3nc(-c4ccc5c(c4)oc4ccncc45)nc(-c4ccc(-n5c6ccccc6c6ccccc65)c5ccccc45)n3)ccc12]

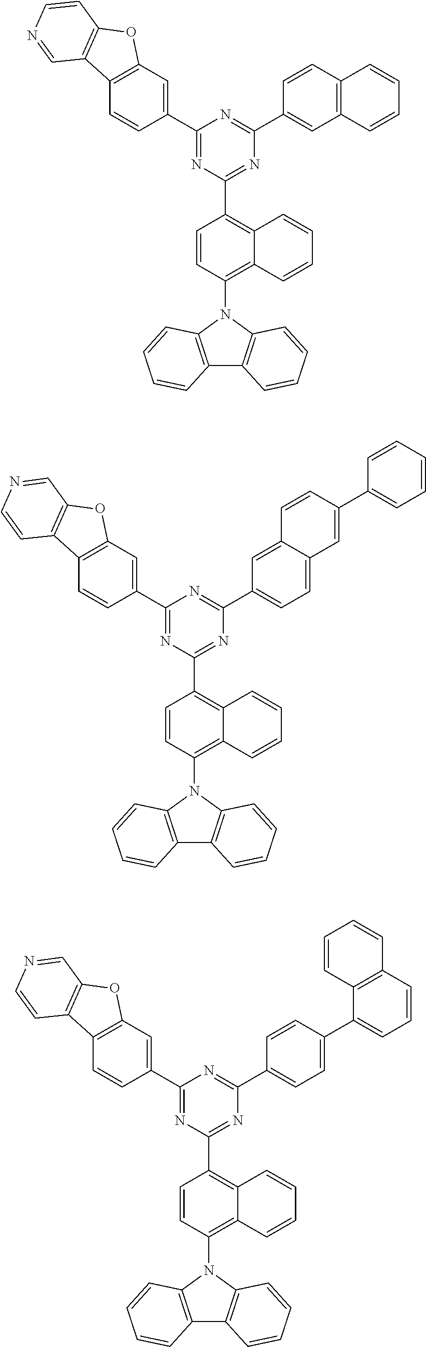 [SMILES: c1ccc2cc(-c3nc(-c4ccc5c(c4)oc4ccncc45)nc(-c4ccc(-n5c6ccccc6c6ccccc65)c5ccccc45)n3)ccc2c1 ; c1ccc(-c2ccc3cc(-c4nc(-c5ccc6c(c5)oc5cnccc56)nc(-c5ccc(-n6c7ccccc7c7ccccc76)c6ccccc56)n4)ccc3c2)cc1 ; c1ccc2c(-c3ccc(-c4nc(-c5ccc6c(c5)oc5cnccc56)nc(-c5ccc(-n6c7ccccc7c7ccccc76)c6ccccc56)n4)cc3)cccc2c1]

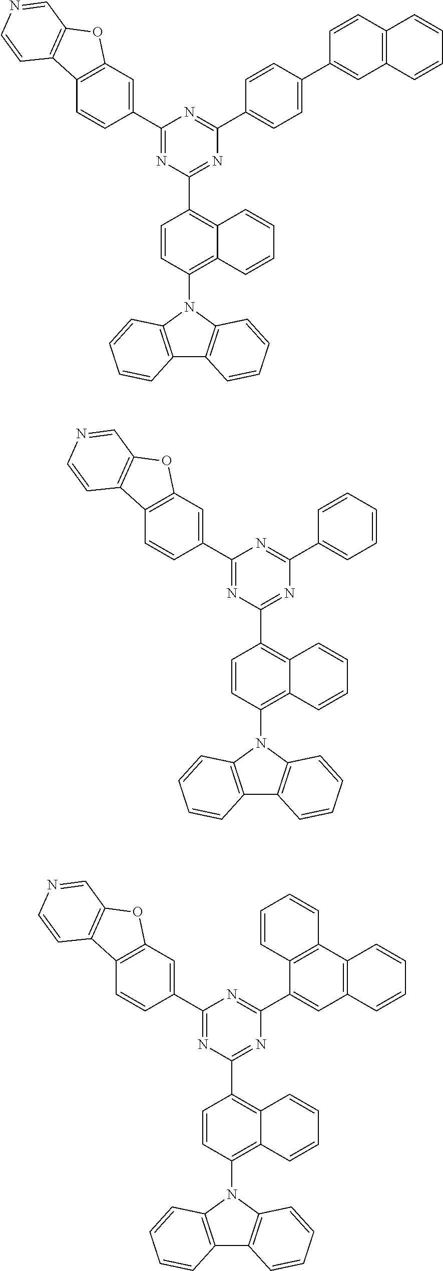 [SMILES: c1ccc2cc(-c3ccc(-c4nc(-c5ccc6c(c5)oc5cnccc56)nc(-c5ccc(-n6c7ccccc7c7ccccc76)c6ccccc56)n4)cc3)ccc2c1 ; c1ccc(-c2nc(-c3ccc4c(c3)oc3cnccc34)nc(-c3ccc(-n4c5ccccc5c5ccccc54)c4ccccc34)n2)cc1 ; c1ccc2c(c1)cc(-c1nc(-c3ccc4c(c3)oc3cnccc34)nc(-c3ccc(-n4c5ccccc5c5ccccc54)c4ccccc34)n1)c1ccccc12]

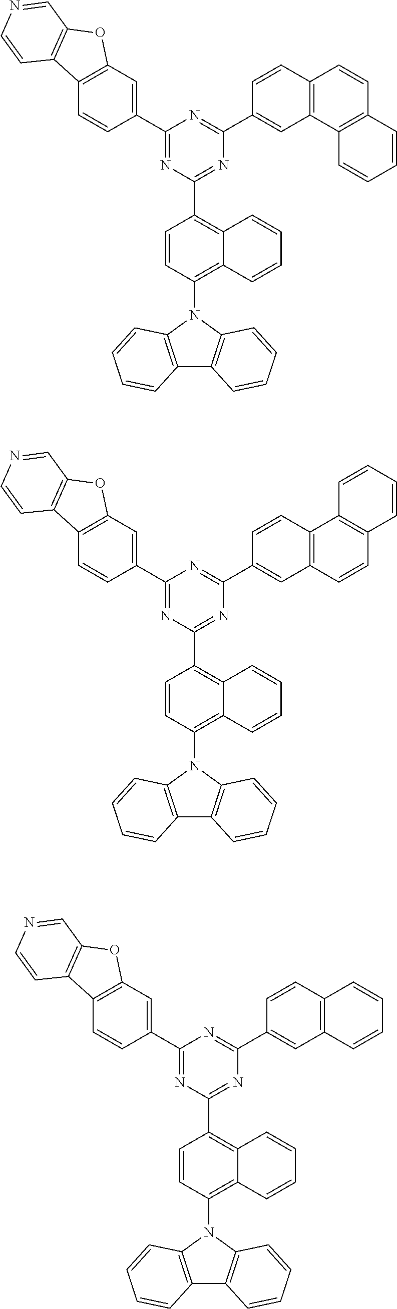 [SMILES: c1ccc2c(c1)ccc1ccc(-c3nc(-c4ccc5c(c4)oc4cnccc45)nc(-c4ccc(-n5c6ccccc6c6ccccc65)c5ccccc45)n3)cc12 ; c1ccc2c(c1)ccc1cc(-c3nc(-c4ccc5c(c4)oc4cnccc45)nc(-c4ccc(-n5c6ccccc6c6ccccc65)c5ccccc45)n3)ccc12 ; c1ccc2cc(-c3nc(-c4ccc5c(c4)oc4cnccc45)nc(-c4ccc(-n5c6ccccc6c6ccccc65)c5ccccc45)n3)ccc2c1]

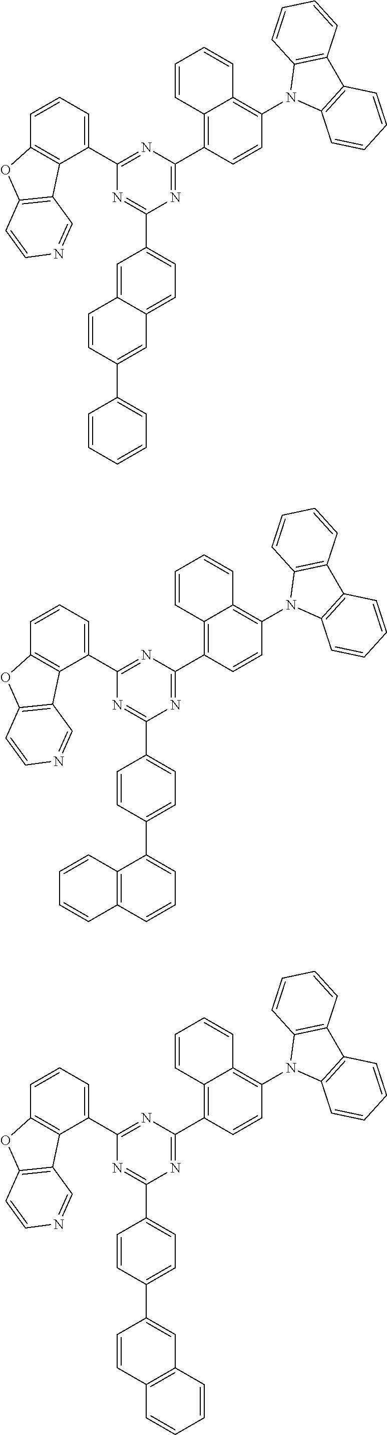 [SMILES: c1ccc(-c2ccc3cc(-c4nc(-c5ccc(-n6c7ccccc7c7ccccc76)c6ccccc56)nc(-c5cccc6oc7ccncc7c56)n4)ccc3c2)cc1 ; c1ccc2c(-c3ccc(-c4nc(-c5ccc(-n6c7ccccc7c7ccccc76)c6ccccc56)nc(-c5cccc6oc7ccncc7c56)n4)cc3)cccc2c1 ; c1ccc2cc(-c3ccc(-c4nc(-c5ccc(-n6c7ccccc7c7ccccc76)c6ccccc56)nc(-c5cccc6oc7ccncc7c56)n4)cc3)ccc2c1]

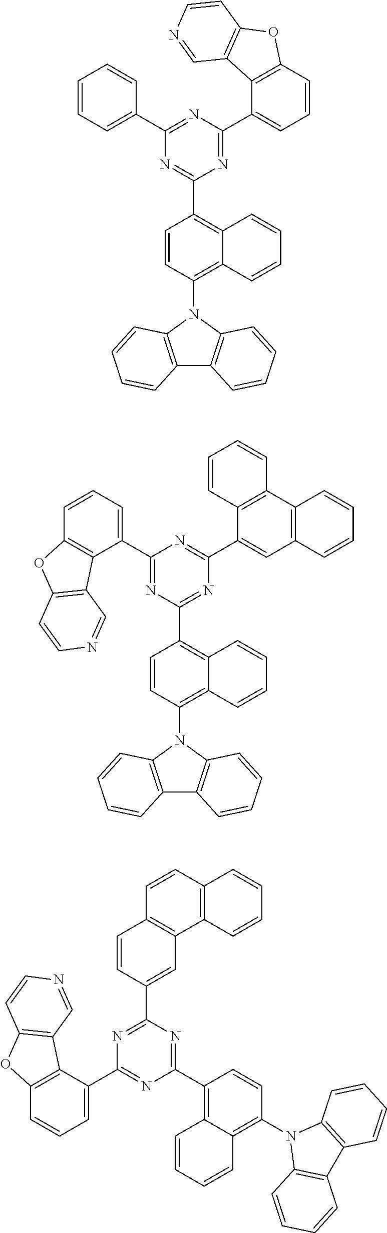 [SMILES: c1ccc(-c2nc(-c3ccc(-n4c5ccccc5c5ccccc54)c4ccccc34)nc(-c3cccc4oc5ccncc5c34)n2)cc1 ; c1ccc2c(c1)cc(-c1nc(-c3ccc(-n4c5ccccc5c5ccccc54)c4ccccc34)nc(-c3cccc4oc5ccncc5c34)n1)c1ccccc12 ; c1ccc2c(c1)ccc1ccc(-c3nc(-c4ccc(-n5c6ccccc6c6ccccc65)c5ccccc45)nc(-c4cccc5oc6ccncc6c45)n3)cc12]

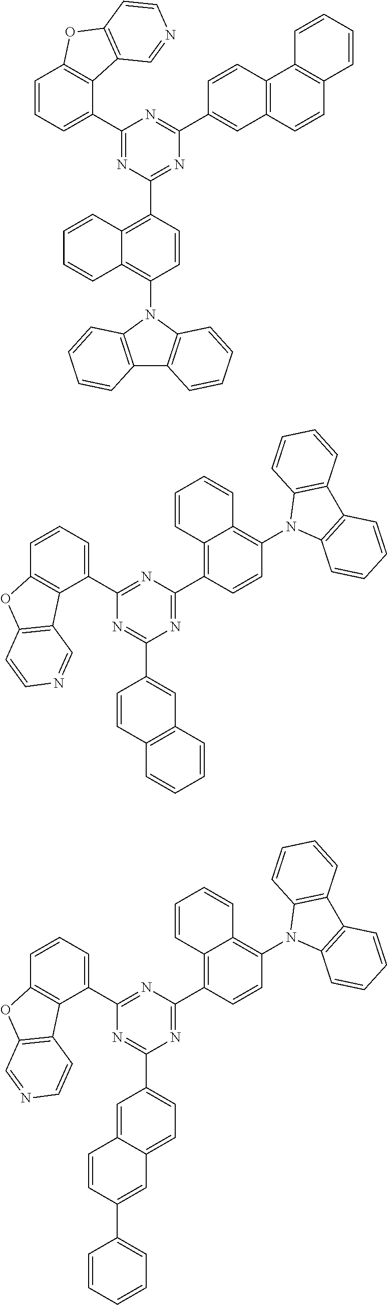 [SMILES: c1ccc2c(c1)ccc1cc(-c3nc(-c4ccc(-n5c6ccccc6c6ccccc65)c5ccccc45)nc(-c4cccc5oc6ccncc6c45)n3)ccc12 ; c1ccc2cc(-c3nc(-c4ccc(-n5c6ccccc6c6ccccc65)c5ccccc45)nc(-c4cccc5oc6ccncc6c45)n3)ccc2c1 ; c1ccc(-c2ccc3cc(-c4nc(-c5ccc(-n6c7ccccc7c7ccccc76)c6ccccc56)nc(-c5cccc6oc7cnccc7c56)n4)ccc3c2)cc1]

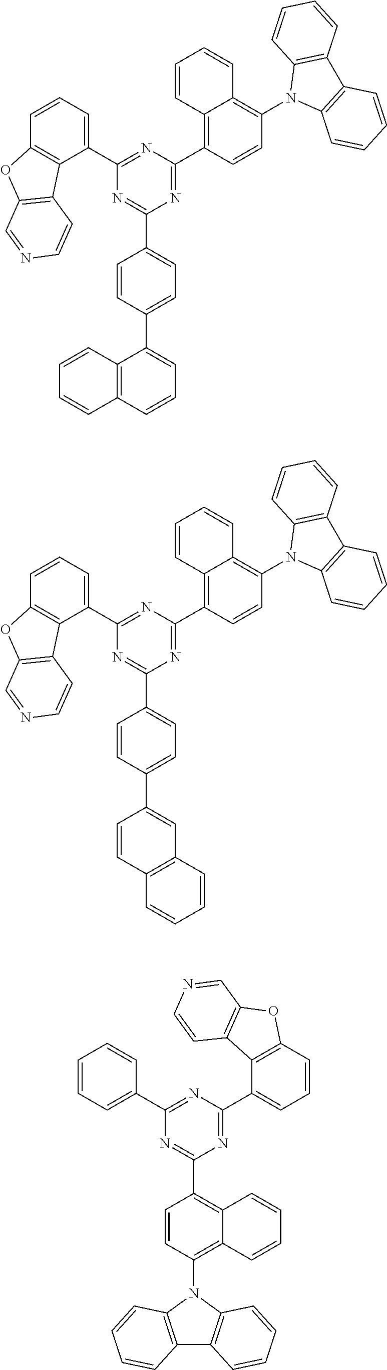 [SMILES: c1ccc2c(-c3ccc(-c4nc(-c5ccc(-n6c7ccccc7c7ccccc76)c6ccccc56)nc(-c5cccc6oc7cnccc7c56)n4)cc3)cccc2c1 ; c1ccc2cc(-c3ccc(-c4nc(-c5ccc(-n6c7ccccc7c7ccccc76)c6ccccc56)nc(-c5cccc6oc7cnccc7c56)n4)cc3)ccc2c1 ; c1ccc(-c2nc(-c3ccc(-n4c5ccccc5c5ccccc54)c4ccccc34)nc(-c3cccc4oc5cnccc5c34)n2)cc1]

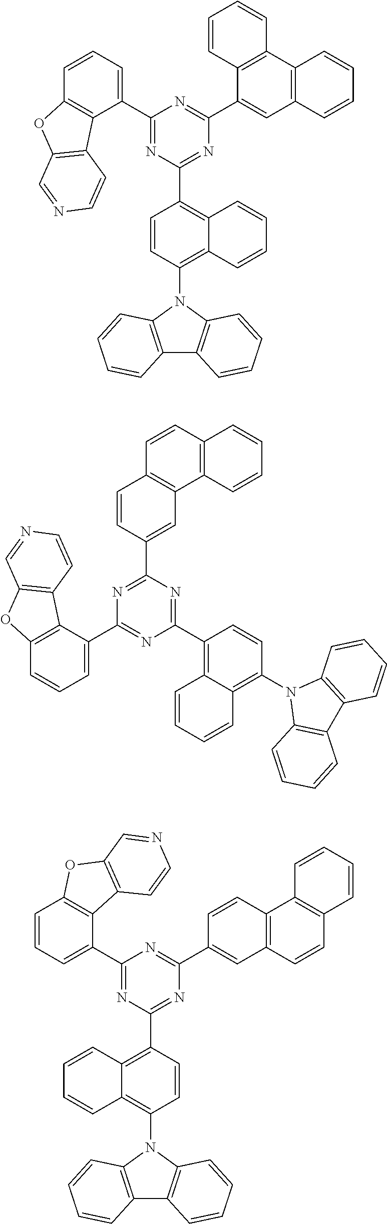 [SMILES: c1ccc2c(c1)cc(-c1nc(-c3ccc(-n4c5ccccc5c5ccccc54)c4ccccc34)nc(-c3cccc4oc5cnccc5c34)n1)c1ccccc12 ; c1ccc2c(c1)ccc1ccc(-c3nc(-c4ccc(-n5c6ccccc6c6ccccc65)c5ccccc45)nc(-c4cccc5oc6cnccc6c45)n3)cc12 ; c1ccc2c(c1)ccc1cc(-c3nc(-c4ccc(-n5c6ccccc6c6ccccc65)c5ccccc45)nc(-c4cccc5oc6cnccc6c45)n3)ccc12]

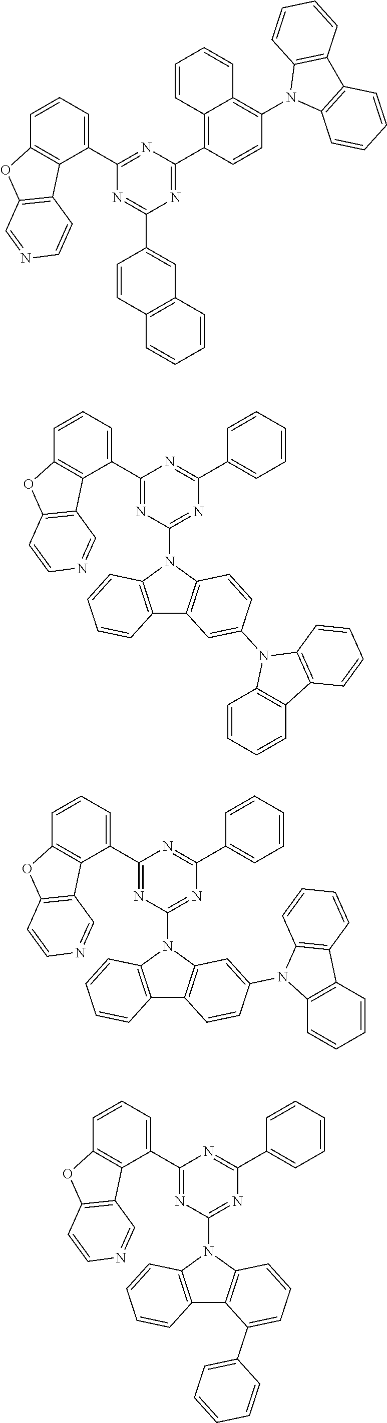

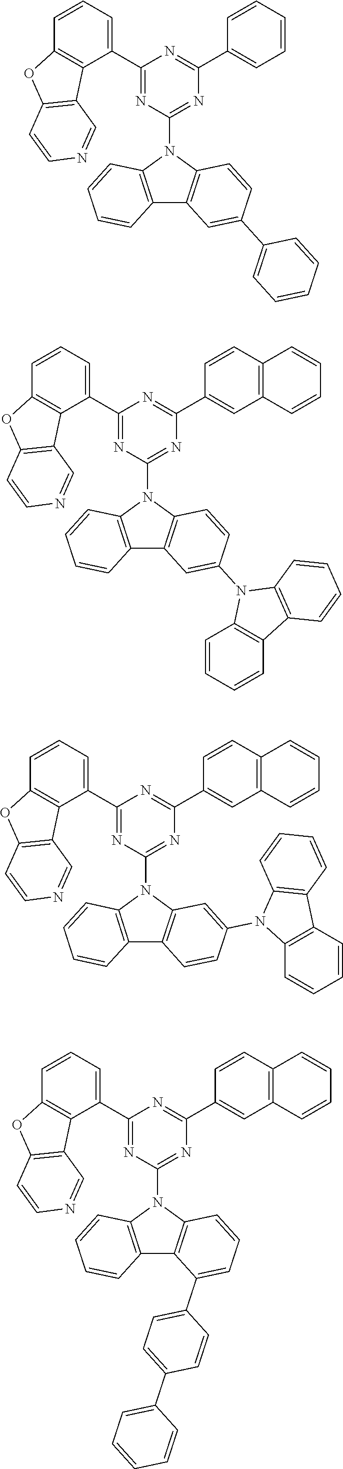 [SMILES: c1ccc(-c2ccc3c(c2)c2ccccc2n3-c2nc(-c3ccccc3)nc(-c3cccc4oc5ccncc5c34)n2)cc1 ; c1ccc2cc(-c3nc(-c4cccc5oc6ccncc6c45)nc(-n4c5ccccc5c5cc(-n6c7ccccc7c7ccccc76)ccc54)n3)ccc2c1 ; c1ccc2cc(-c3nc(-c4cccc5oc6ccncc6c45)nc(-n4c5ccccc5c5ccc(-n6c7ccccc7c7ccccc76)cc54)n3)ccc2c1 ; c1ccc(-c2ccc(-c3cccc4c3c3ccccc3n4-c3nc(-c4ccc5ccccc5c4)nc(-c4cccc5oc6ccncc6c45)n3)cc2)cc1]

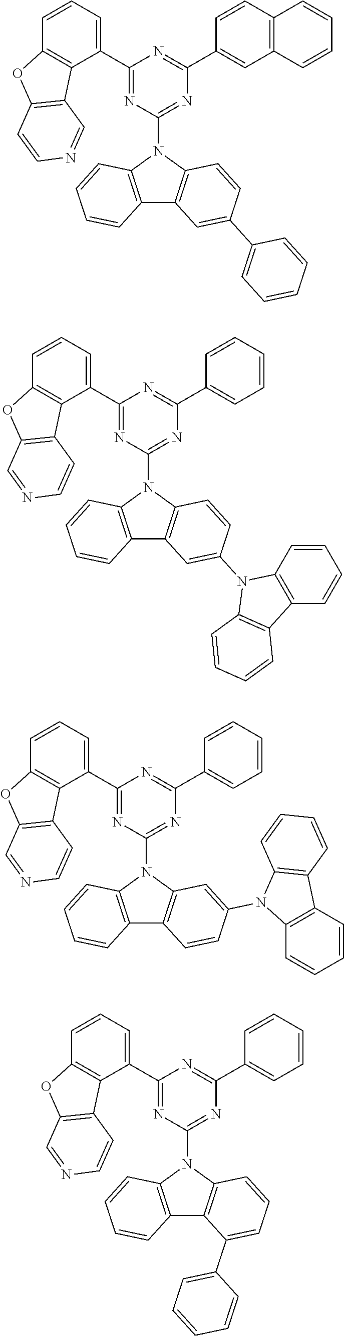

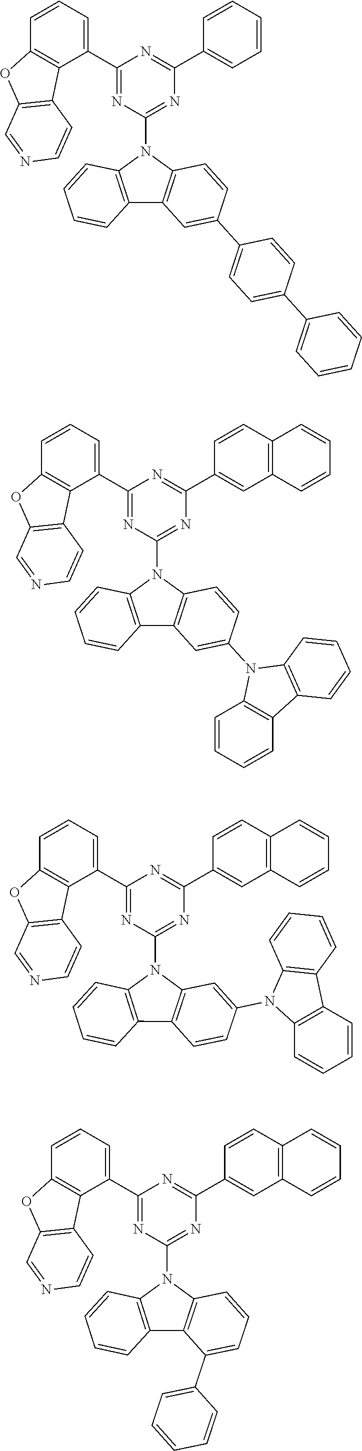 [SMILES: c1ccc(-c2ccc(-c3ccc4c(c3)c3ccccc3n4-c3nc(-c4ccccc4)nc(-c4cccc5oc6cnccc6c45)n3)cc2)cc1 ; c1ccc2cc(-c3nc(-c4cccc5oc6cnccc6c45)nc(-n4c5ccccc5c5cc(-n6c7ccccc7c7ccccc76)ccc54)n3)ccc2c1 ; c1ccc2cc(-c3nc(-c4cccc5oc6cnccc6c45)nc(-n4c5ccccc5c5ccc(-n6c7ccccc7c7ccccc76)cc54)n3)ccc2c1 ; c1ccc(-c2cccc3c2c2ccccc2n3-c2nc(-c3ccc4ccccc4c3)nc(-c3cccc4oc5cnccc5c34)n2)cc1]

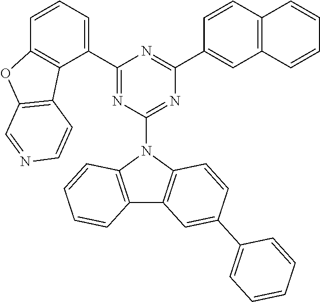 [SMILES: c1ccc(-c2ccc3c(c2)c2ccccc2n3-c2nc(-c3ccc4ccccc4c3)nc(-c3cccc4oc5cnccc5c34)n2)cc1]

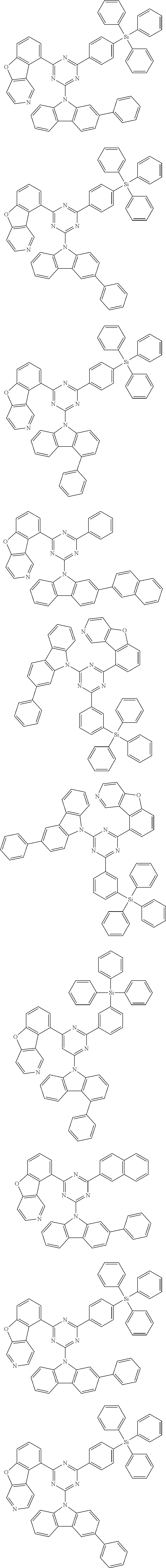

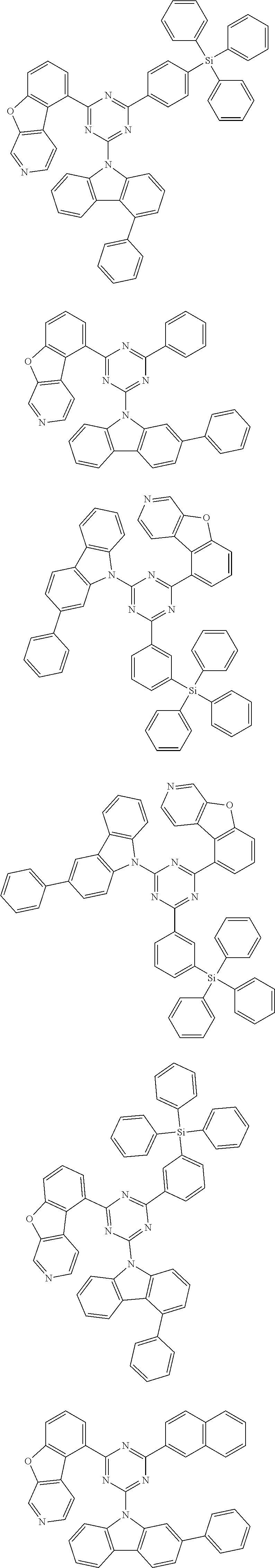

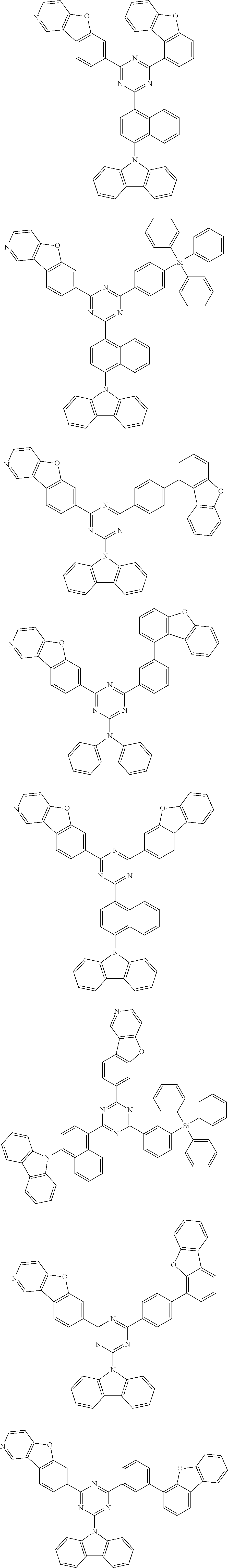

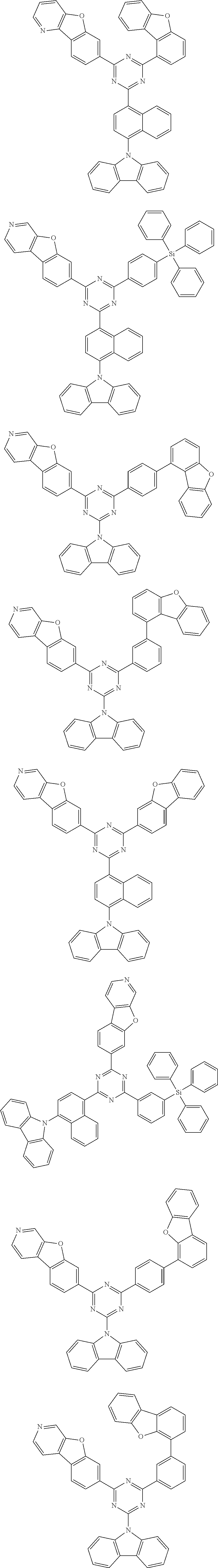

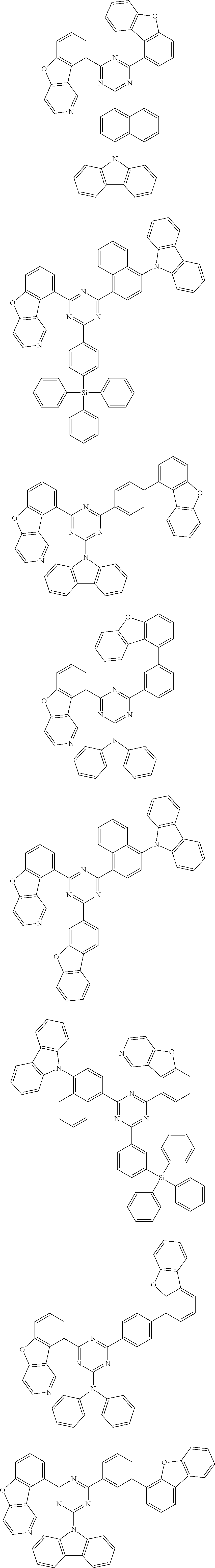

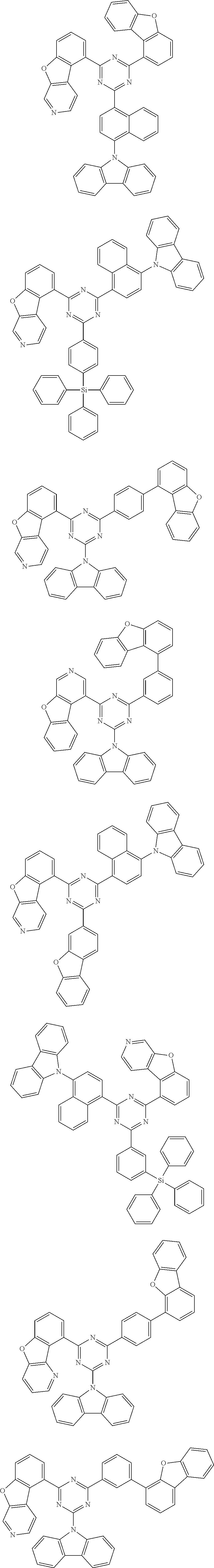

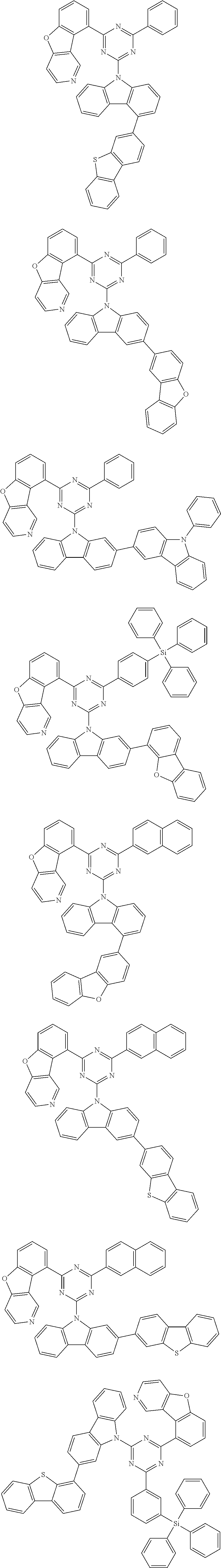

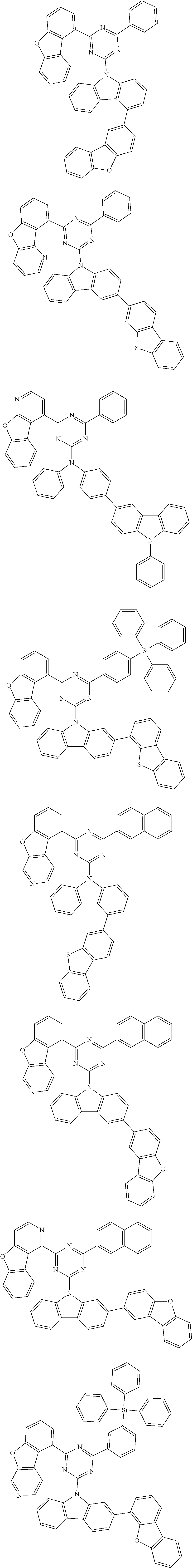

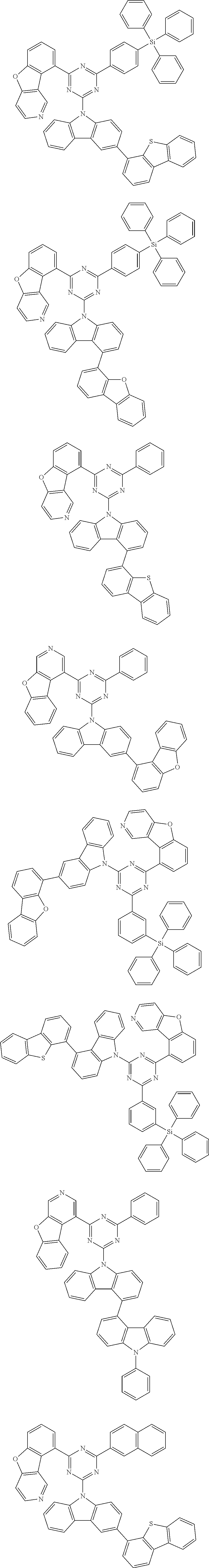

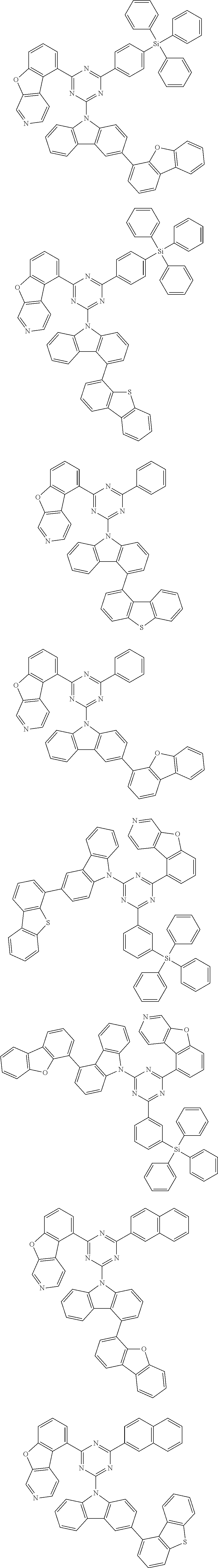

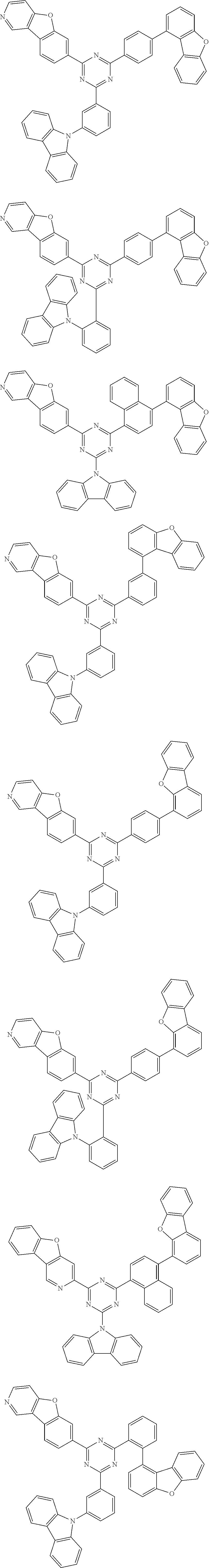

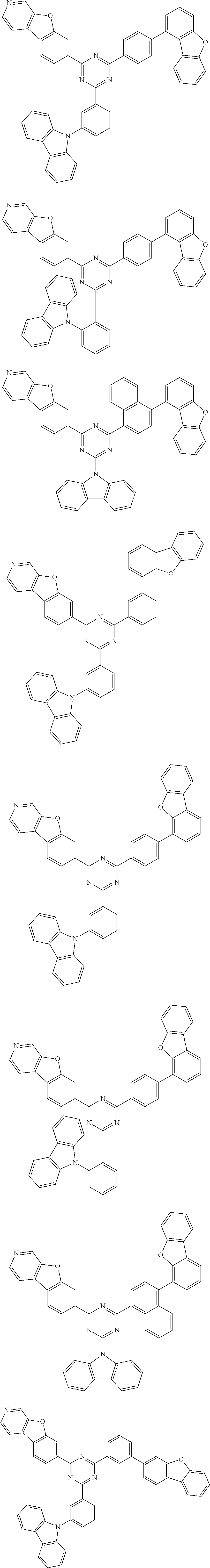

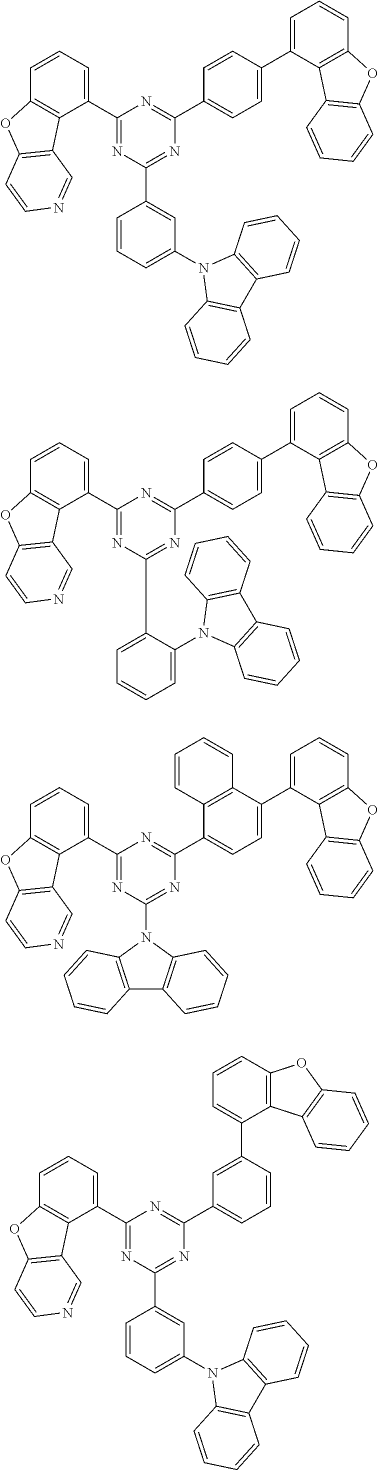 [SMILES: c1cc(-c2nc(-c3ccc(-c4cccc5oc6ccccc6c45)cc3)nc(-c3cccc4oc5ccncc5c34)n2)cc(-n2c3ccccc3c3ccccc32)c1 ; c1ccc(-n2c3ccccc3c3ccccc32)c(-c2nc(-c3ccc(-c4cccc5oc6ccccc6c45)cc3)nc(-c3cccc4oc5ccncc5c34)n2)c1 ; c1ccc2c(c1)oc1cccc(-c3ccc(-c4nc(-c5cccc6oc7ccncc7c56)nc(-n5c6ccccc6c6ccccc65)n4)c4ccccc34)c12 ; c1cc(-c2nc(-c3cccc(-n4c5ccccc5c5ccccc54)c3)nc(-c3cccc4oc5ccncc5c34)n2)cc(-c2cccc3oc4ccccc4c23)c1]

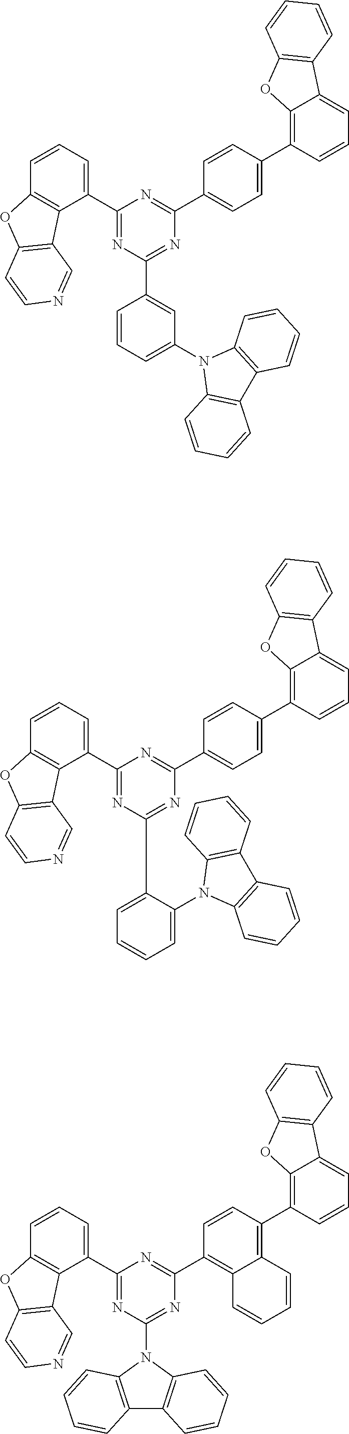 [SMILES: c1cc(-c2nc(-c3ccc(-c4cccc5c4oc4ccccc45)cc3)nc(-c3cccc4oc5ccncc5c34)n2)cc(-n2c3ccccc3c3ccccc32)c1 ; c1ccc(-n2c3ccccc3c3ccccc32)c(-c2nc(-c3ccc(-c4cccc5c4oc4ccccc45)cc3)nc(-c3cccc4oc5ccncc5c34)n2)c1 ; c1ccc2c(c1)oc1c(-c3ccc(-c4nc(-c5cccc6oc7ccncc7c56)nc(-n5c6ccccc6c6ccccc65)n4)c4ccccc34)cccc12]

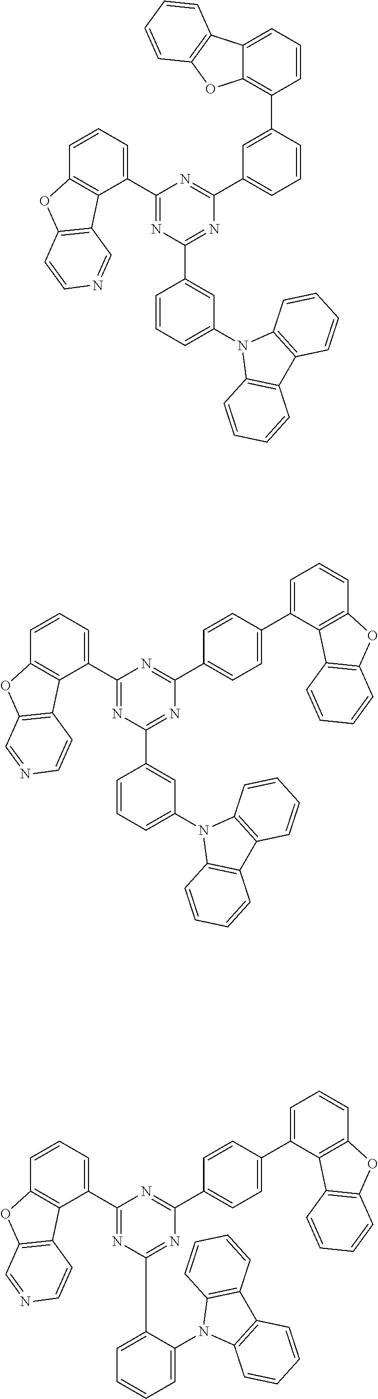 [SMILES: c1cc(-c2nc(-c3cccc(-n4c5ccccc5c5ccccc54)c3)nc(-c3cccc4oc5ccncc5c34)n2)cc(-c2cccc3c2oc2ccccc23)c1 ; c1cc(-c2nc(-c3ccc(-c4cccc5oc6ccccc6c45)cc3)nc(-c3cccc4oc5cnccc5c34)n2)cc(-n2c3ccccc3c3ccccc32)c1 ; c1ccc(-n2c3ccccc3c3ccccc32)c(-c2nc(-c3ccc(-c4cccc5oc6ccccc6c45)cc3)nc(-c3cccc4oc5cnccc5c34)n2)c1]

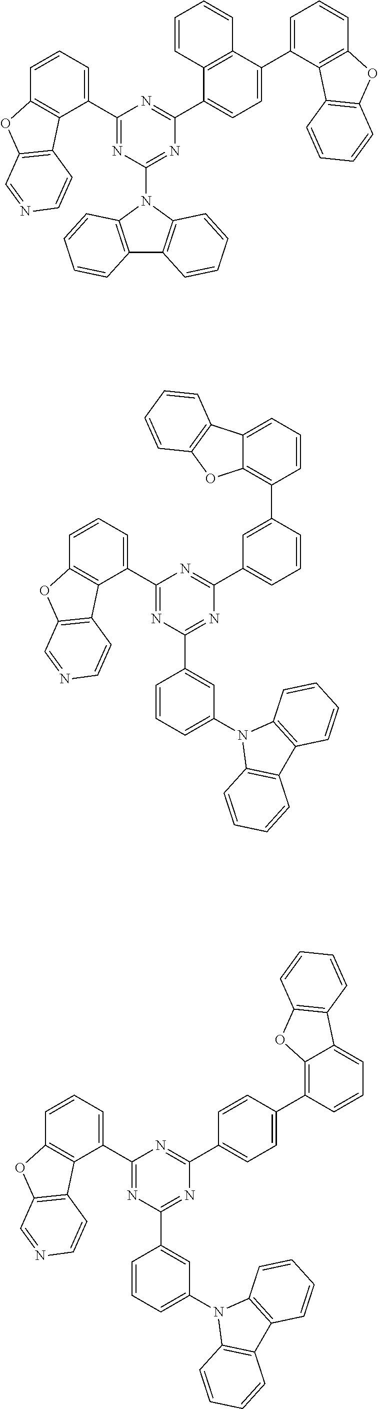 [SMILES: c1ccc2c(c1)oc1cccc(-c3ccc(-c4nc(-c5cccc6oc7cnccc7c56)nc(-n5c6ccccc6c6ccccc65)n4)c4ccccc34)c12 ; c1cc(-c2nc(-c3cccc(-n4c5ccccc5c5ccccc54)c3)nc(-c3cccc4oc5cnccc5c34)n2)cc(-c2cccc3c2oc2ccccc23)c1 ; c1cc(-c2nc(-c3ccc(-c4cccc5c4oc4ccccc45)cc3)nc(-c3cccc4oc5cnccc5c34)n2)cc(-n2c3ccccc3c3ccccc32)c1]

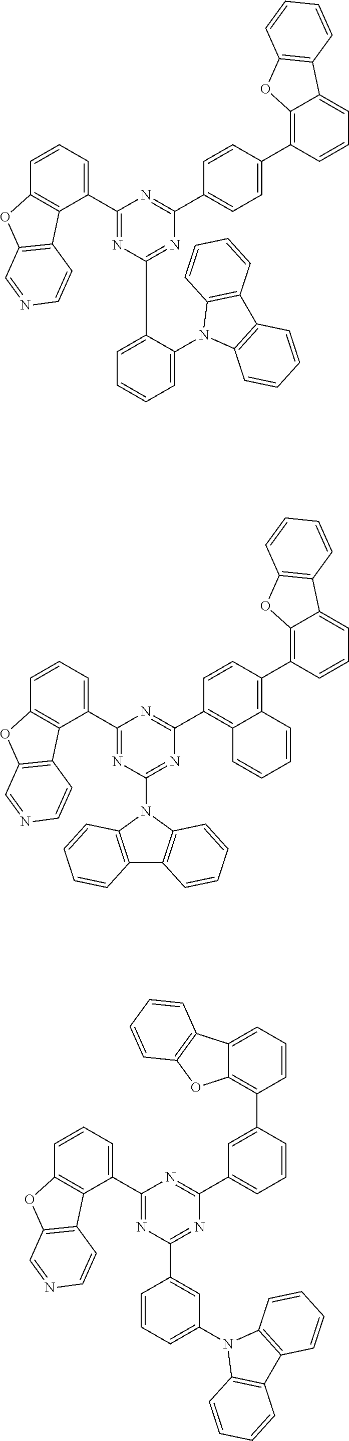 [SMILES: c1ccc(-n2c3ccccc3c3ccccc32)c(-c2nc(-c3ccc(-c4cccc5c4oc4ccccc45)cc3)nc(-c3cccc4oc5cnccc5c34)n2)c1 ; c1ccc2c(c1)oc1c(-c3ccc(-c4nc(-c5cccc6oc7cnccc7c56)nc(-n5c6ccccc6c6ccccc65)n4)c4ccccc34)cccc12 ; c1cc(-c2nc(-c3cccc(-n4c5ccccc5c5ccccc54)c3)nc(-c3cccc4oc5cnccc5c34)n2)cc(-c2cccc3c2oc2ccccc23)c1]

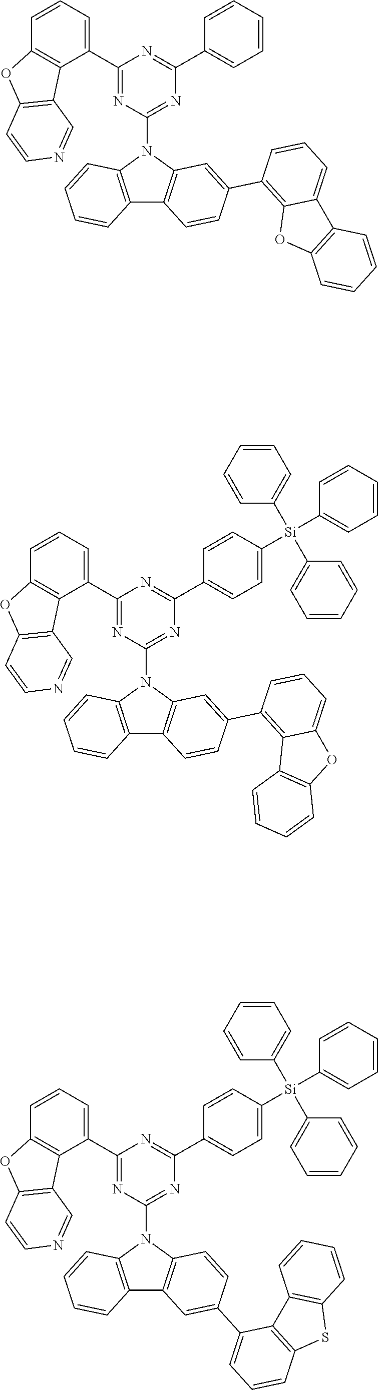 [SMILES: c1ccc(-c2nc(-c3cccc4oc5ccncc5c34)nc(-n3c4ccccc4c4ccc(-c5cccc6c5oc5ccccc56)cc43)n2)cc1 ; c1ccc([Si](c2ccccc2)(c2ccccc2)c2ccc(-c3nc(-c4cccc5oc6ccncc6c45)nc(-n4c5ccccc5c5ccc(-c6cccc7oc8ccccc8c67)cc54)n3)cc2)cc1 ; c1ccc([Si](c2ccccc2)(c2ccccc2)c2ccc(-c3nc(-c4cccc5oc6ccncc6c45)nc(-n4c5ccccc5c5cc(-c6cccc7sc8ccccc8c67)ccc54)n3)cc2)cc1]

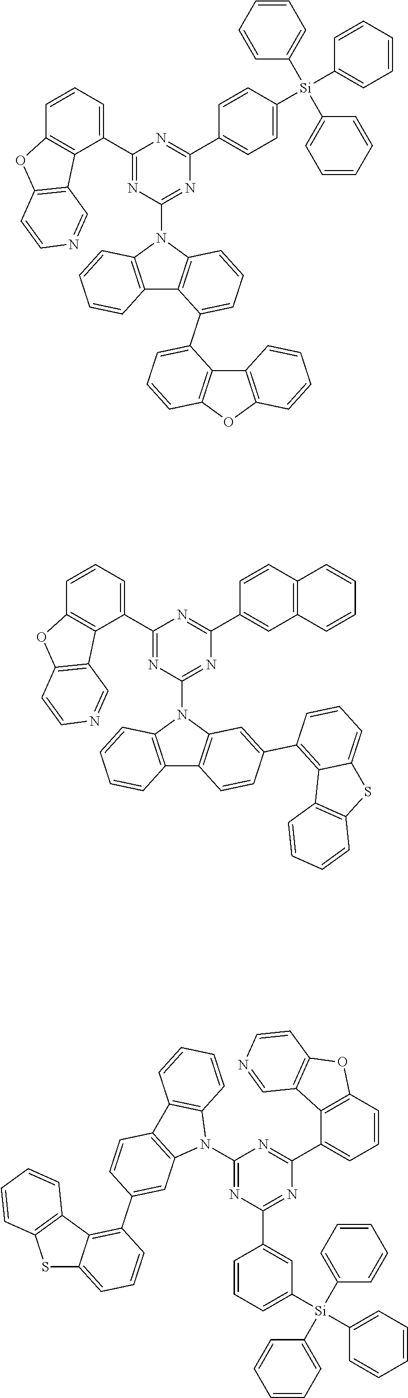 [SMILES: c1ccc([Si](c2ccccc2)(c2ccccc2)c2ccc(-c3nc(-c4cccc5oc6ccncc6c45)nc(-n4c5ccccc5c5c(-c6cccc7oc8ccccc8c67)cccc54)n3)cc2)cc1 ; c1ccc2cc(-c3nc(-c4cccc5oc6ccncc6c45)nc(-n4c5ccccc5c5ccc(-c6cccc7sc8ccccc8c67)cc54)n3)ccc2c1 ; c1ccc([Si](c2ccccc2)(c2ccccc2)c2cccc(-c3nc(-c4cccc5oc6ccncc6c45)nc(-n4c5ccccc5c5ccc(-c6cccc7sc8ccccc8c67)cc54)n3)c2)cc1]

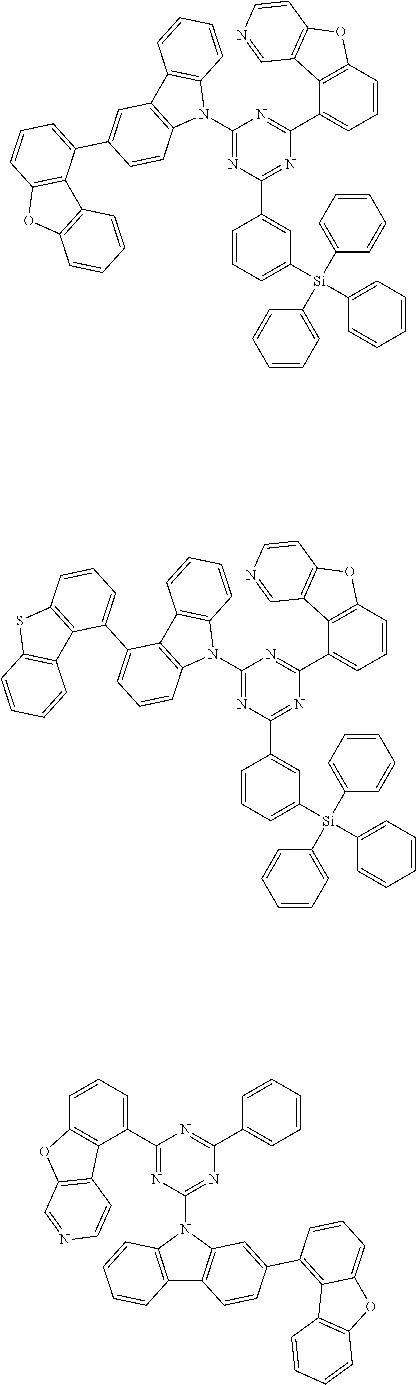 [SMILES: c1ccc([Si](c2ccccc2)(c2ccccc2)c2cccc(-c3nc(-c4cccc5oc6ccncc6c45)nc(-n4c5ccccc5c5cc(-c6cccc7oc8ccccc8c67)ccc54)n3)c2)cc1 ; c1ccc([Si](c2ccccc2)(c2ccccc2)c2cccc(-c3nc(-c4cccc5oc6ccncc6c45)nc(-n4c5ccccc5c5c(-c6cccc7sc8ccccc8c67)cccc54)n3)c2)cc1 ; c1ccc(-c2nc(-c3cccc4oc5cnccc5c34)nc(-n3c4ccccc4c4ccc(-c5cccc6oc7ccccc7c56)cc43)n2)cc1]

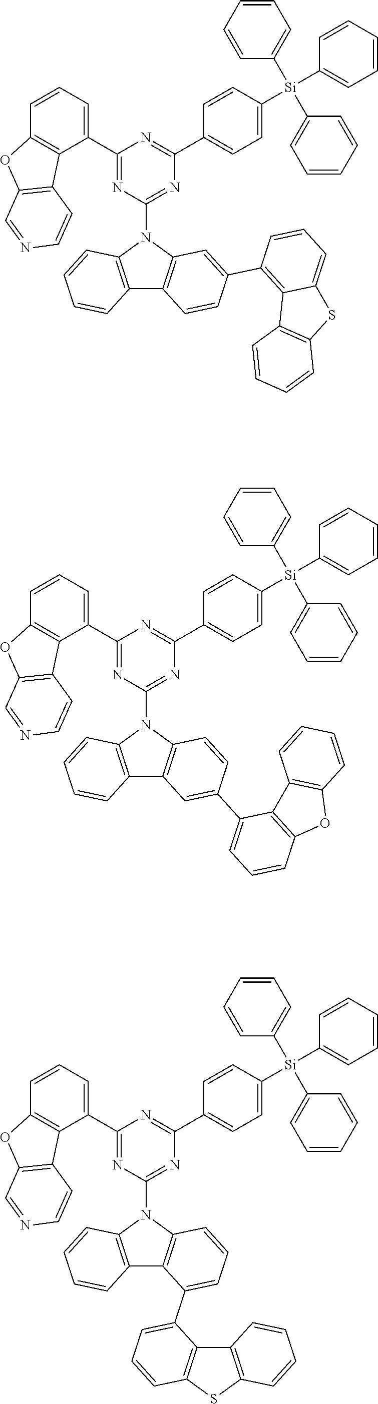 [SMILES: c1ccc([Si](c2ccccc2)(c2ccccc2)c2ccc(-c3nc(-c4cccc5oc6cnccc6c45)nc(-n4c5ccccc5c5ccc(-c6cccc7sc8ccccc8c67)cc54)n3)cc2)cc1 ; c1ccc([Si](c2ccccc2)(c2ccccc2)c2ccc(-c3nc(-c4cccc5oc6cnccc6c45)nc(-n4c5ccccc5c5cc(-c6cccc7oc8ccccc8c67)ccc54)n3)cc2)cc1 ; c1ccc([Si](c2ccccc2)(c2ccccc2)c2ccc(-c3nc(-c4cccc5oc6cnccc6c45)nc(-n4c5ccccc5c5c(-c6cccc7sc8ccccc8c67)cccc54)n3)cc2)cc1]

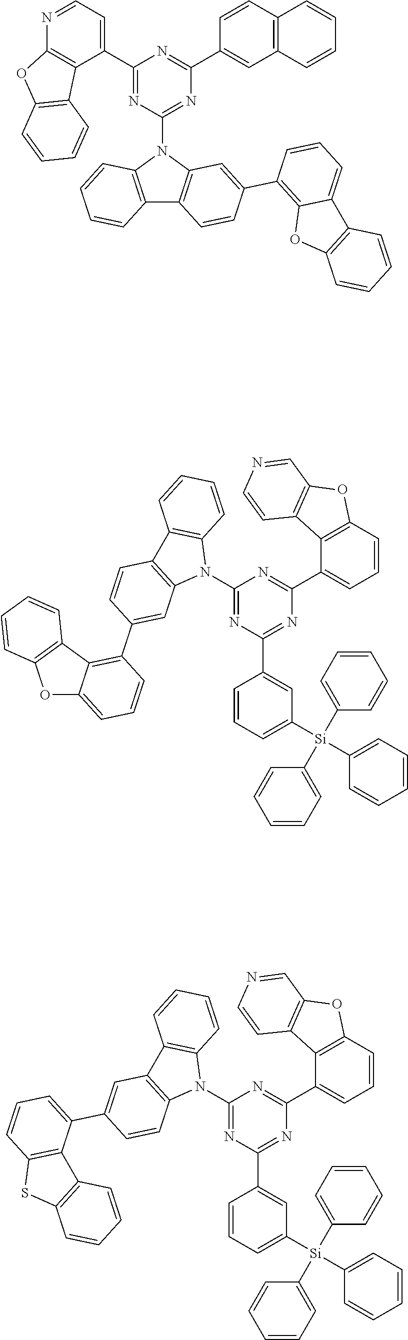 [SMILES: c1ccc2cc(-c3nc(-c4ccnc5oc6ccccc6c45)nc(-n4c5ccccc5c5ccc(-c6cccc7c6oc6ccccc67)cc54)n3)ccc2c1 ; c1ccc([Si](c2ccccc2)(c2ccccc2)c2cccc(-c3nc(-c4cccc5oc6cnccc6c45)nc(-n4c5ccccc5c5ccc(-c6cccc7oc8ccccc8c67)cc54)n3)c2)cc1 ; c1ccc([Si](c2ccccc2)(c2ccccc2)c2cccc(-c3nc(-c4cccc5oc6cnccc6c45)nc(-n4c5ccccc5c5cc(-c6cccc7sc8ccccc8c67)ccc54)n3)c2)cc1]

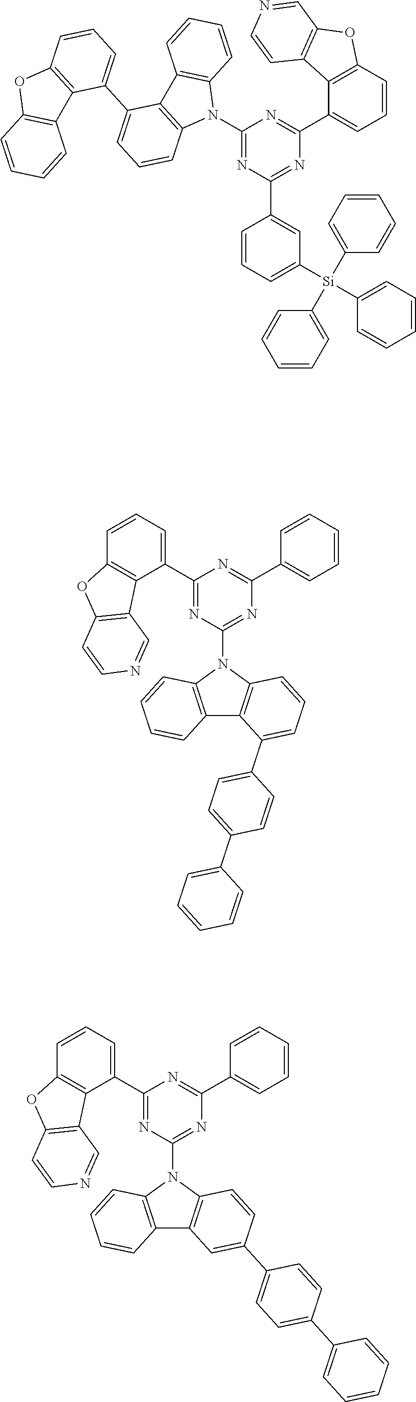 [SMILES: c1ccc([Si](c2ccccc2)(c2ccccc2)c2cccc(-c3nc(-c4cccc5oc6cnccc6c45)nc(-n4c5ccccc5c5c(-c6cccc7oc8ccccc8c67)cccc54)n3)c2)cc1 ; c1ccc(-c2ccc(-c3cccc4c3c3ccccc3n4-c3nc(-c4ccccc4)nc(-c4cccc5oc6ccncc6c45)n3)cc2)cc1 ; c1ccc(-c2ccc(-c3ccc4c(c3)c3ccccc3n4-c3nc(-c4ccccc4)nc(-c4cccc5oc6ccncc6c45)n3)cc2)cc1]

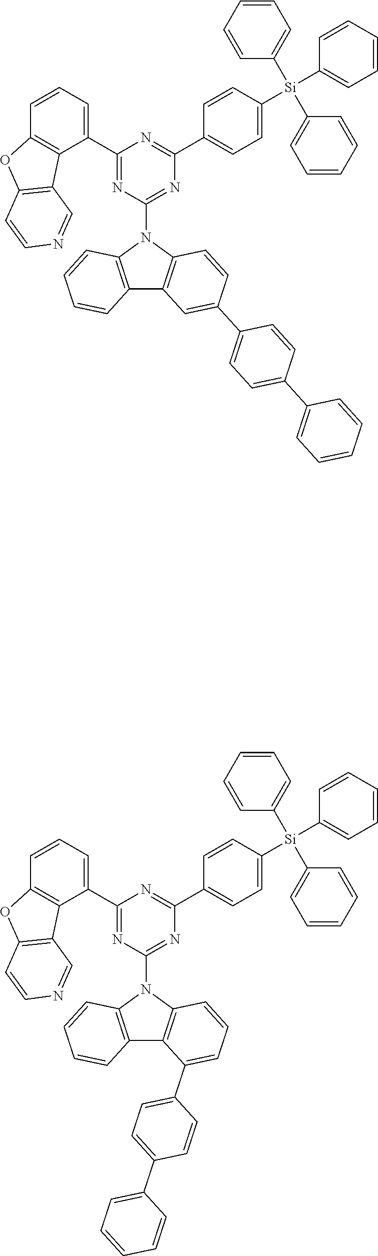 [SMILES: c1ccc(-c2ccc(-c3ccc4c(c3)c3ccccc3n4-c3nc(-c4ccc([Si](c5ccccc5)(c5ccccc5)c5ccccc5)cc4)nc(-c4cccc5oc6ccncc6c45)n3)cc2)cc1 ; c1ccc(-c2ccc(-c3cccc4c3c3ccccc3n4-c3nc(-c4ccc([Si](c5ccccc5)(c5ccccc5)c5ccccc5)cc4)nc(-c4cccc5oc6ccncc6c45)n3)cc2)cc1]

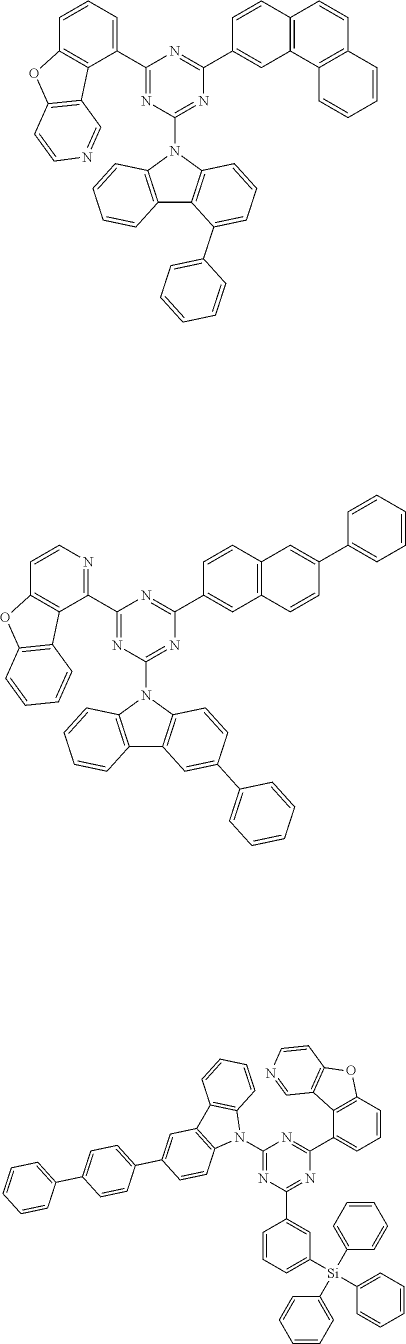 [SMILES: c1ccc(-c2cccc3c2c2ccccc2n3-c2nc(-c3ccc4ccc5ccccc5c4c3)nc(-c3cccc4oc5ccncc5c34)n2)cc1 ; c1ccc(-c2ccc3cc(-c4nc(-c5nccc6oc7ccccc7c56)nc(-n5c6ccccc6c6cc(-c7ccccc7)ccc65)n4)ccc3c2)cc1 ; c1ccc(-c2ccc(-c3ccc4c(c3)c3ccccc3n4-c3nc(-c4cccc([Si](c5ccccc5)(c5ccccc5)c5ccccc5)c4)nc(-c4cccc5oc6ccncc6c45)n3)cc2)cc1]

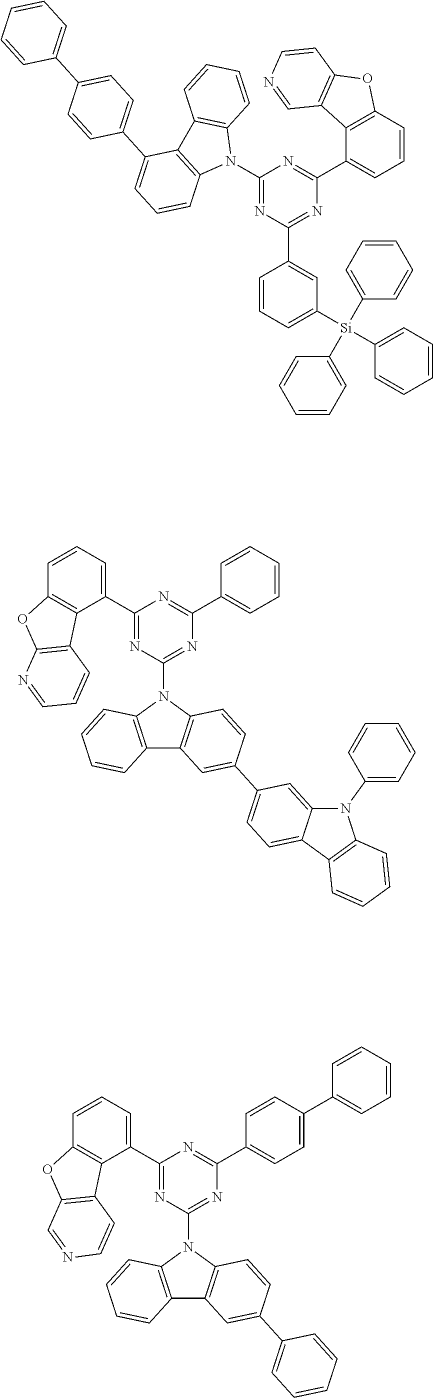 [SMILES: c1ccc(-c2ccc(-c3cccc4c3c3ccccc3n4-c3nc(-c4cccc([Si](c5ccccc5)(c5ccccc5)c5ccccc5)c4)nc(-c4cccc5oc6ccncc6c45)n3)cc2)cc1 ; c1ccc(-c2nc(-c3cccc4oc5ncccc5c34)nc(-n3c4ccccc4c4cc(-c5ccc6c7ccccc7n(-c7ccccc7)c6c5)ccc43)n2)cc1 ; c1ccc(-c2ccc(-c3nc(-c4cccc5oc6cnccc6c45)nc(-n4c5ccccc5c5cc(-c6ccccc6)ccc54)n3)cc2)cc1]

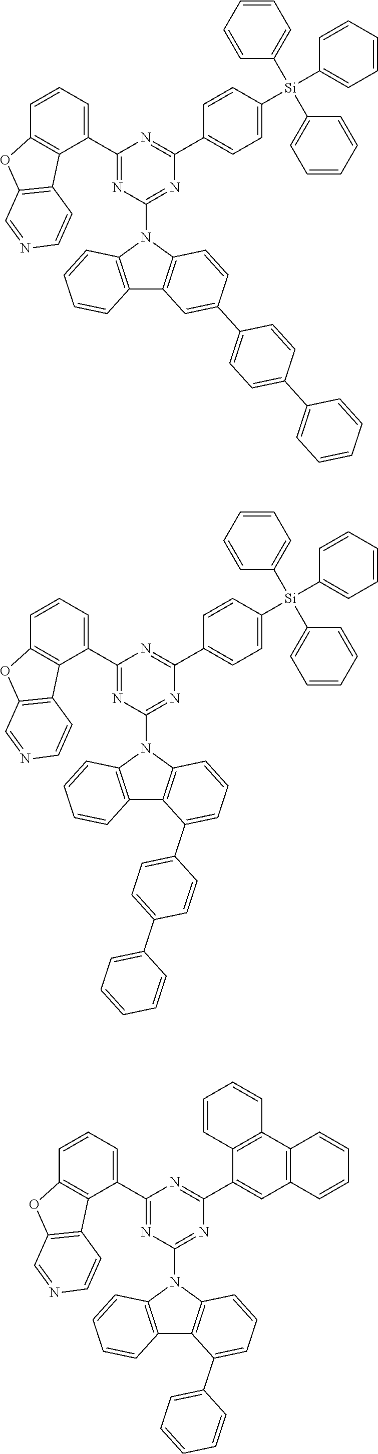 [SMILES: c1ccc(-c2ccc(-c3ccc4c(c3)c3ccccc3n4-c3nc(-c4ccc([Si](c5ccccc5)(c5ccccc5)c5ccccc5)cc4)nc(-c4cccc5oc6cnccc6c45)n3)cc2)cc1 ; c1ccc(-c2ccc(-c3cccc4c3c3ccccc3n4-c3nc(-c4ccc([Si](c5ccccc5)(c5ccccc5)c5ccccc5)cc4)nc(-c4cccc5oc6cnccc6c45)n3)cc2)cc1 ; c1ccc(-c2cccc3c2c2ccccc2n3-c2nc(-c3cc4ccccc4c4ccccc34)nc(-c3cccc4oc5cnccc5c34)n2)cc1]

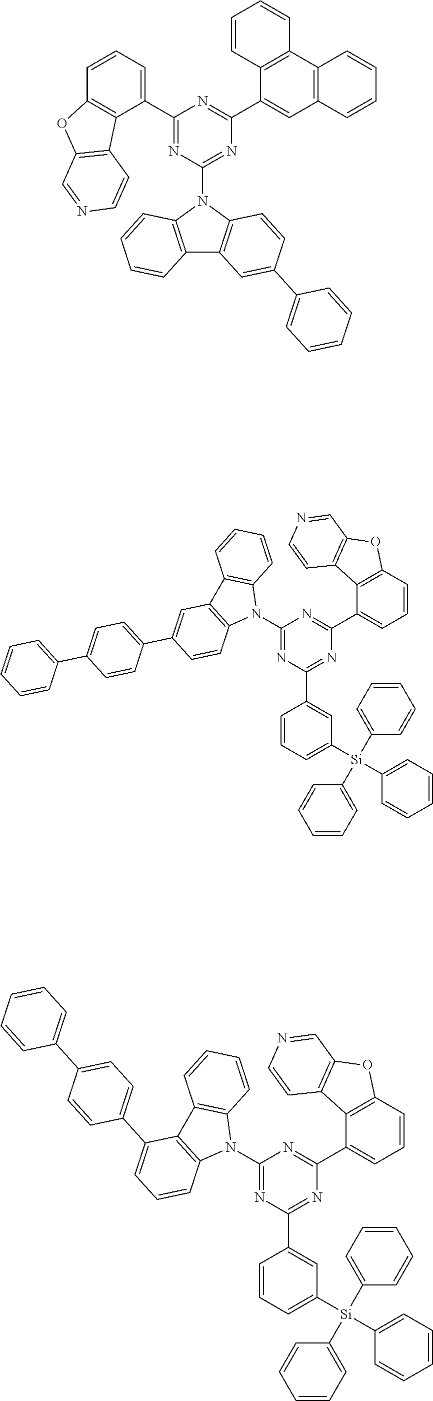 [SMILES: c1ccc(-c2ccc3c(c2)c2ccccc2n3-c2nc(-c3cc4ccccc4c4ccccc34)nc(-c3cccc4oc5cnccc5c34)n2)cc1 ; c1ccc(-c2ccc(-c3ccc4c(c3)c3ccccc3n4-c3nc(-c4cccc([Si](c5ccccc5)(c5ccccc5)c5ccccc5)c4)nc(-c4cccc5oc6cnccc6c45)n3)cc2)cc1 ; c1ccc(-c2ccc(-c3cccc4c3c3ccccc3n4-c3nc(-c4cccc([Si](c5ccccc5)(c5ccccc5)c5ccccc5)c4)nc(-c4cccc5oc6cnccc6c45)n3)cc2)cc1]

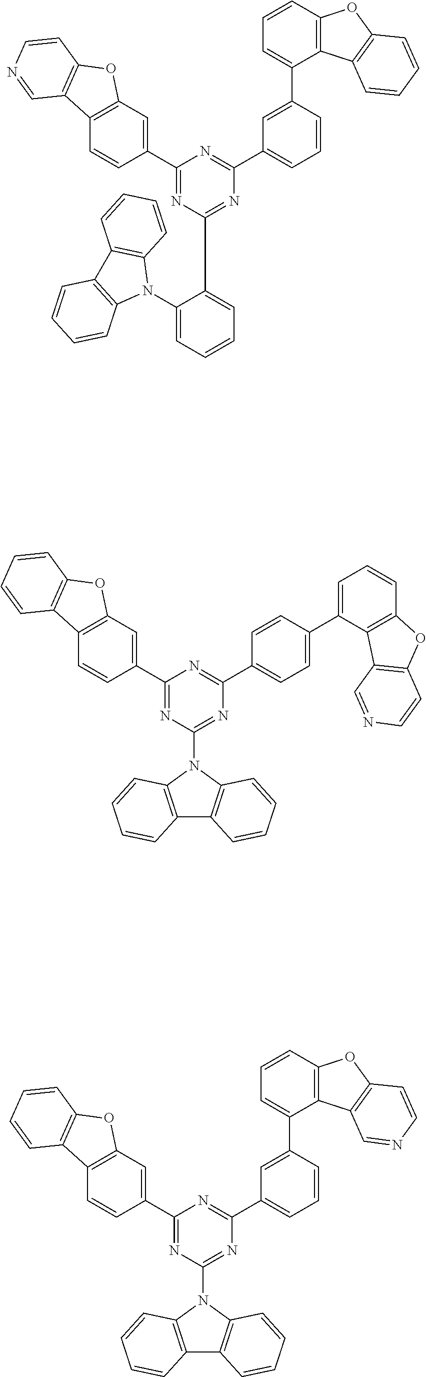 [SMILES: c1cc(-c2nc(-c3ccc4c(c3)oc3ccncc34)nc(-c3ccccc3-n3c4ccccc4c4ccccc43)n2)cc(-c2cccc3oc4ccccc4c23)c1 ; c1ccc2c(c1)oc1cc(-c3nc(-c4ccc(-c5cccc6oc7ccncc7c56)cc4)nc(-n4c5ccccc5c5ccccc54)n3)ccc12 ; c1cc(-c2nc(-c3ccc4c(c3)oc3ccccc34)nc(-n3c4ccccc4c4ccccc43)n2)cc(-c2cccc3oc4ccncc4c23)c1]

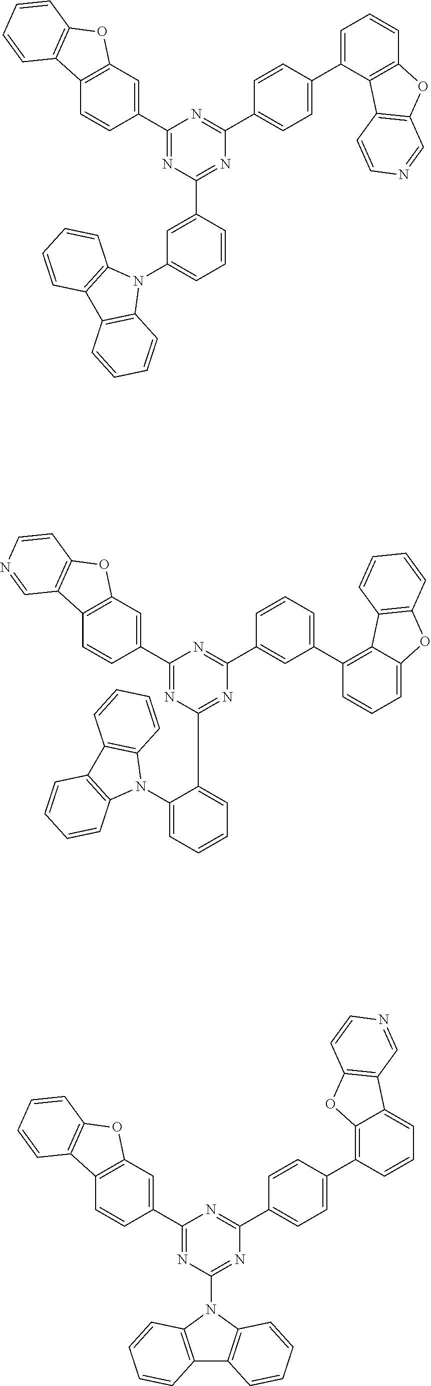 [SMILES: c1cc(-c2nc(-c3ccc(-c4cccc5oc6cnccc6c45)cc3)nc(-c3ccc4c(c3)oc3ccccc34)n2)cc(-n2c3ccccc3c3ccccc32)c1 ; c1cc(-c2nc(-c3ccc4c(c3)oc3ccncc34)nc(-c3ccccc3-n3c4ccccc4c4ccccc43)n2)cc(-c2cccc3oc4ccccc4c23)c1 ; c1ccc2c(c1)oc1cc(-c3nc(-c4ccc(-c5cccc6c5oc5ccncc56)cc4)nc(-n4c5ccccc5c5ccccc54)n3)ccc12]

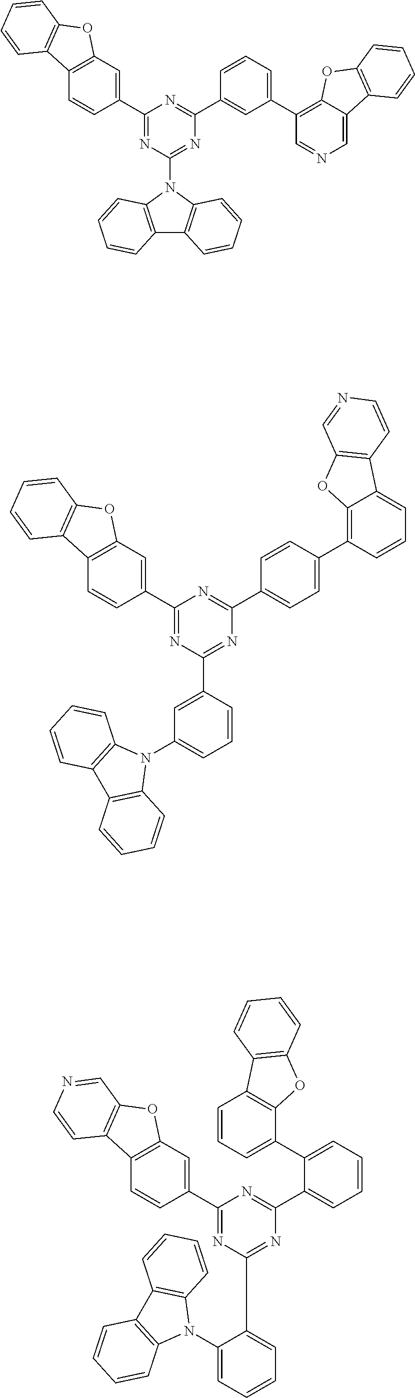 [SMILES: c1cc(-c2nc(-c3ccc4c(c3)oc3ccccc34)nc(-n3c4ccccc4c4ccccc43)n2)cc(-c2cncc3c2oc2ccccc23)c1 ; c1cc(-c2nc(-c3ccc(-c4cccc5c4oc4cnccc45)cc3)nc(-c3ccc4c(c3)oc3ccccc34)n2)cc(-n2c3ccccc3c3ccccc32)c1 ; c1ccc(-c2cccc3c2oc2ccccc23)c(-c2nc(-c3ccc4c(c3)oc3cnccc34)nc(-c3ccccc3-n3c4ccccc4c4ccccc43)n2)c1]

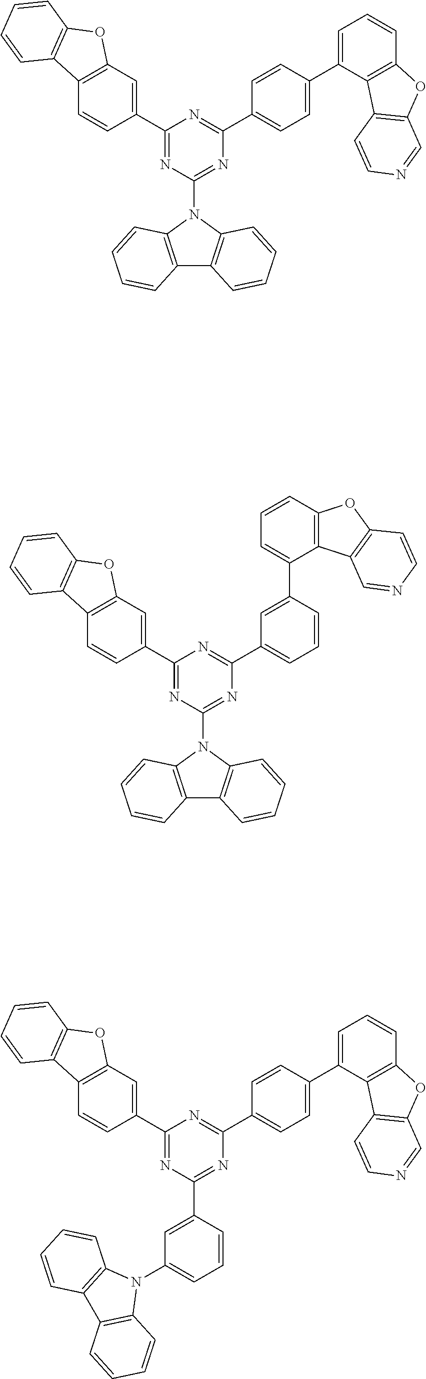 [SMILES: c1ccc2c(c1)oc1cc(-c3nc(-c4ccc(-c5cccc6oc7cnccc7c56)cc4)nc(-n4c5ccccc5c5ccccc54)n3)ccc12 ; c1cc(-c2nc(-c3ccc4c(c3)oc3ccccc34)nc(-n3c4ccccc4c4ccccc43)n2)cc(-c2cccc3oc4ccncc4c23)c1 ; c1cc(-c2nc(-c3ccc(-c4cccc5oc6cnccc6c45)cc3)nc(-c3ccc4c(c3)oc3ccccc34)n2)cc(-n2c3ccccc3c3ccccc32)c1]

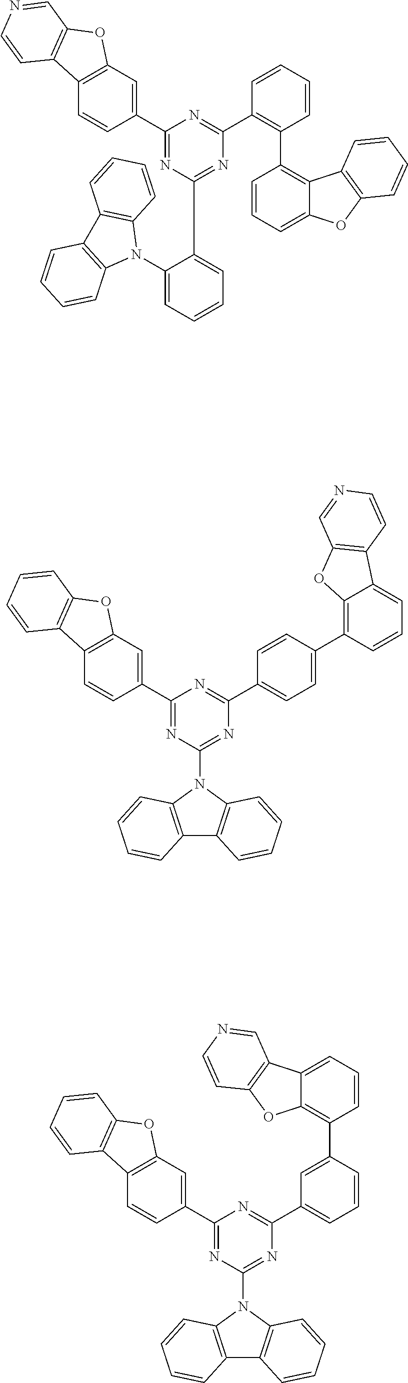 [SMILES: c1ccc(-c2cccc3oc4ccccc4c23)c(-c2nc(-c3ccc4c(c3)oc3cnccc34)nc(-c3ccccc3-n3c4ccccc4c4ccccc43)n2)c1 ; c1ccc2c(c1)oc1cc(-c3nc(-c4ccc(-c5cccc6c5oc5cnccc56)cc4)nc(-n4c5ccccc5c5ccccc54)n3)ccc12 ; c1cc(-c2nc(-c3ccc4c(c3)oc3ccccc34)nc(-n3c4ccccc4c4ccccc43)n2)cc(-c2cccc3c2oc2ccncc23)c1]

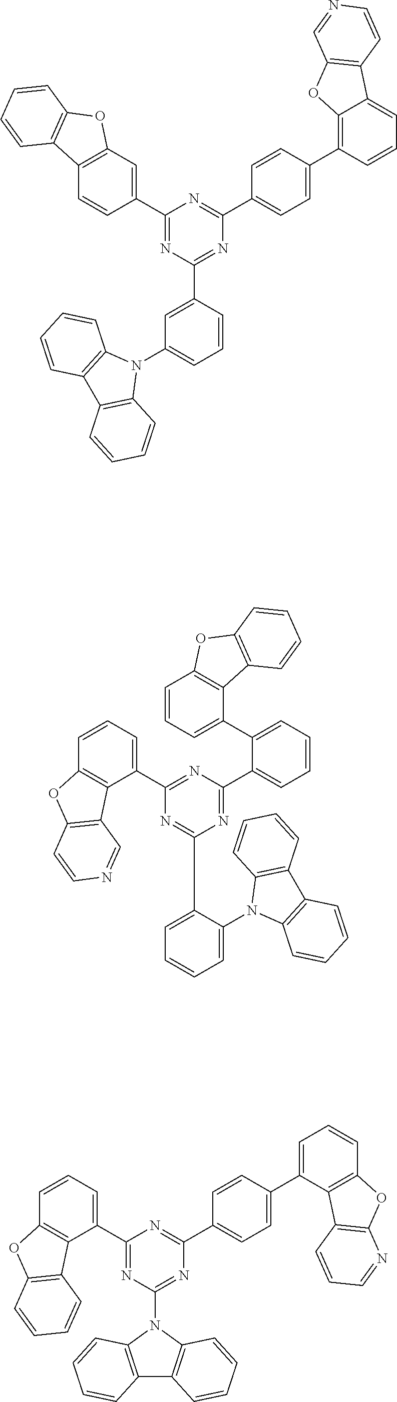 [SMILES: c1cc(-c2nc(-c3ccc(-c4cccc5c4oc4cnccc45)cc3)nc(-c3ccc4c(c3)oc3ccccc34)n2)cc(-n2c3ccccc3c3ccccc32)c1 ; c1ccc(-c2cccc3oc4ccccc4c23)c(-c2nc(-c3ccccc3-n3c4ccccc4c4ccccc43)nc(-c3cccc4oc5ccncc5c34)n2)c1 ; c1ccc2c(c1)oc1cccc(-c3nc(-c4ccc(-c5cccc6oc7ncccc7c56)cc4)nc(-n4c5ccccc5c5ccccc54)n3)c12]

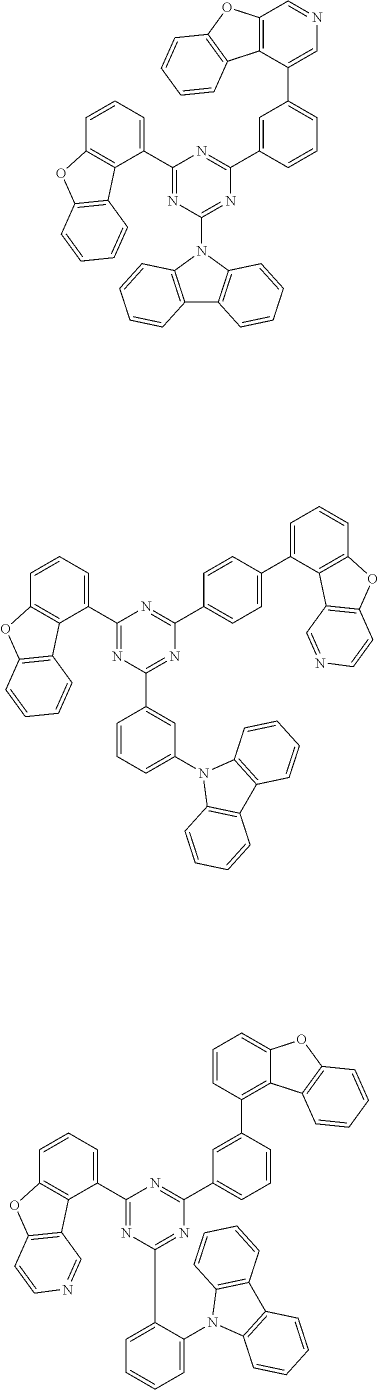 [SMILES: c1cc(-c2nc(-c3cccc4oc5ccccc5c34)nc(-n3c4ccccc4c4ccccc43)n2)cc(-c2cncc3oc4ccccc4c23)c1 ; c1cc(-c2nc(-c3ccc(-c4cccc5oc6ccncc6c45)cc3)nc(-c3cccc4oc5ccccc5c34)n2)cc(-n2c3ccccc3c3ccccc32)c1 ; c1cc(-c2nc(-c3ccccc3-n3c4ccccc4c4ccccc43)nc(-c3cccc4oc5ccncc5c34)n2)cc(-c2cccc3oc4ccccc4c23)c1]

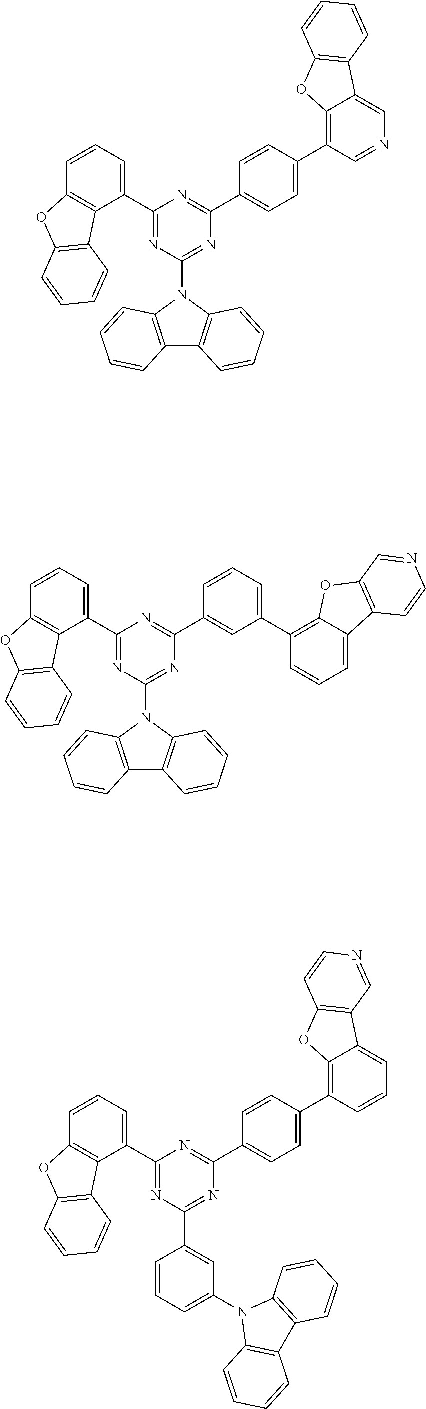 [SMILES: c1ccc2c(c1)oc1c(-c3ccc(-c4nc(-c5cccc6oc7ccccc7c56)nc(-n5c6ccccc6c6ccccc65)n4)cc3)cncc12 ; c1cc(-c2nc(-c3cccc4oc5ccccc5c34)nc(-n3c4ccccc4c4ccccc43)n2)cc(-c2cccc3c2oc2cnccc23)c1 ; c1cc(-c2nc(-c3ccc(-c4cccc5c4oc4ccncc45)cc3)nc(-c3cccc4oc5ccccc5c34)n2)cc(-n2c3ccccc3c3ccccc32)c1]

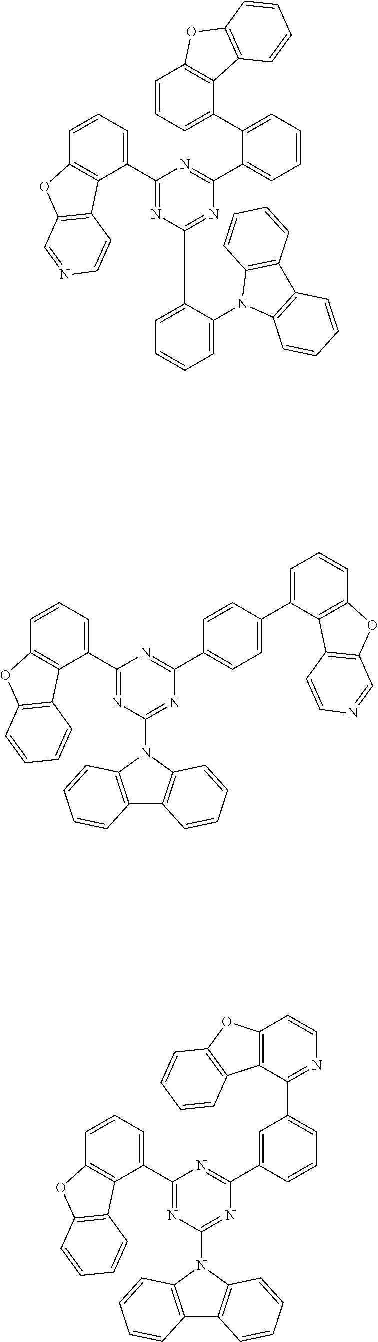 [SMILES: c1ccc(-c2cccc3oc4ccccc4c23)c(-c2nc(-c3ccccc3-n3c4ccccc4c4ccccc43)nc(-c3cccc4oc5cnccc5c34)n2)c1 ; c1ccc2c(c1)oc1cccc(-c3nc(-c4ccc(-c5cccc6oc7cnccc7c56)cc4)nc(-n4c5ccccc5c5ccccc54)n3)c12 ; c1cc(-c2nc(-c3cccc4oc5ccccc5c34)nc(-n3c4ccccc4c4ccccc43)n2)cc(-c2nccc3oc4ccccc4c23)c1]

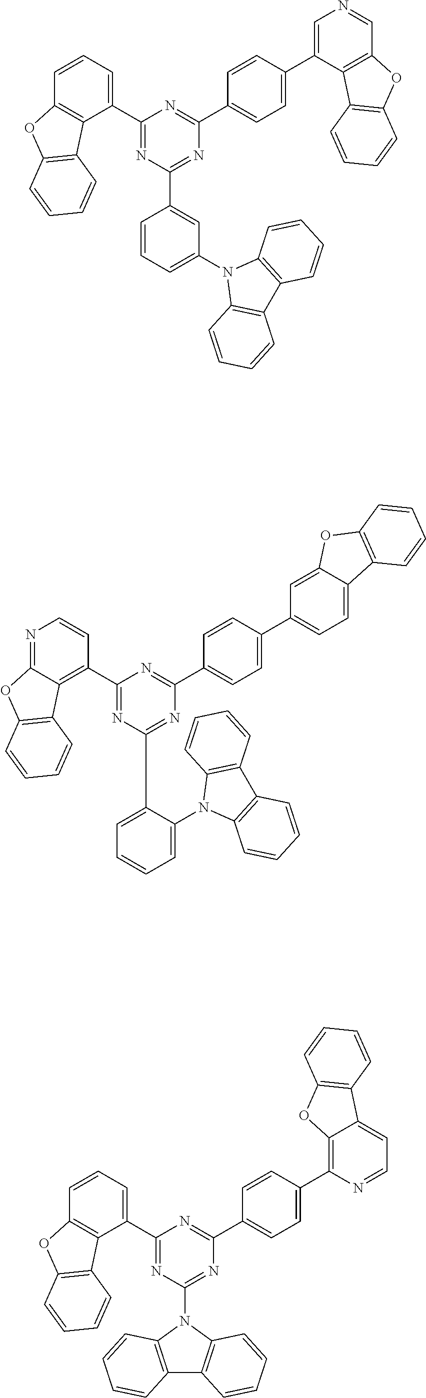 [SMILES: c1cc(-c2nc(-c3ccc(-c4cncc5oc6ccccc6c45)cc3)nc(-c3cccc4oc5ccccc5c34)n2)cc(-n2c3ccccc3c3ccccc32)c1 ; c1ccc(-n2c3ccccc3c3ccccc32)c(-c2nc(-c3ccc(-c4ccc5c(c4)oc4ccccc45)cc3)nc(-c3ccnc4oc5ccccc5c34)n2)c1 ; c1ccc2c(c1)oc1c(-c3ccc(-c4nc(-c5cccc6oc7ccccc7c56)nc(-n5c6ccccc6c6ccccc65)n4)cc3)nccc12]

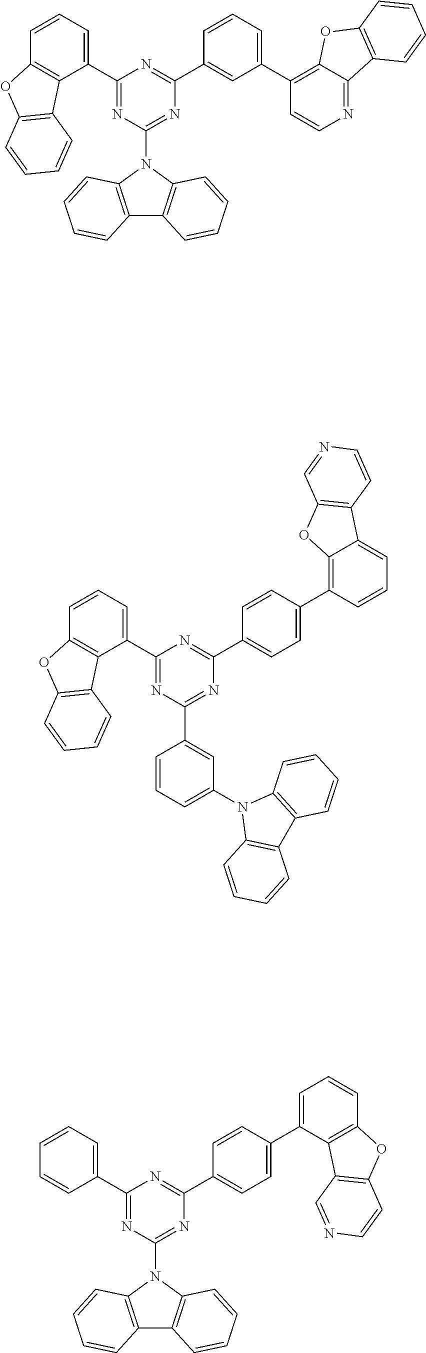 [SMILES: c1cc(-c2nc(-c3cccc4oc5ccccc5c34)nc(-n3c4ccccc4c4ccccc43)n2)cc(-c2ccnc3c2oc2ccccc23)c1 ; c1cc(-c2nc(-c3ccc(-c4cccc5c4oc4cnccc45)cc3)nc(-c3cccc4oc5ccccc5c34)n2)cc(-n2c3ccccc3c3ccccc32)c1 ; c1ccc(-c2nc(-c3ccc(-c4cccc5oc6ccncc6c45)cc3)nc(-n3c4ccccc4c4ccccc43)n2)cc1]

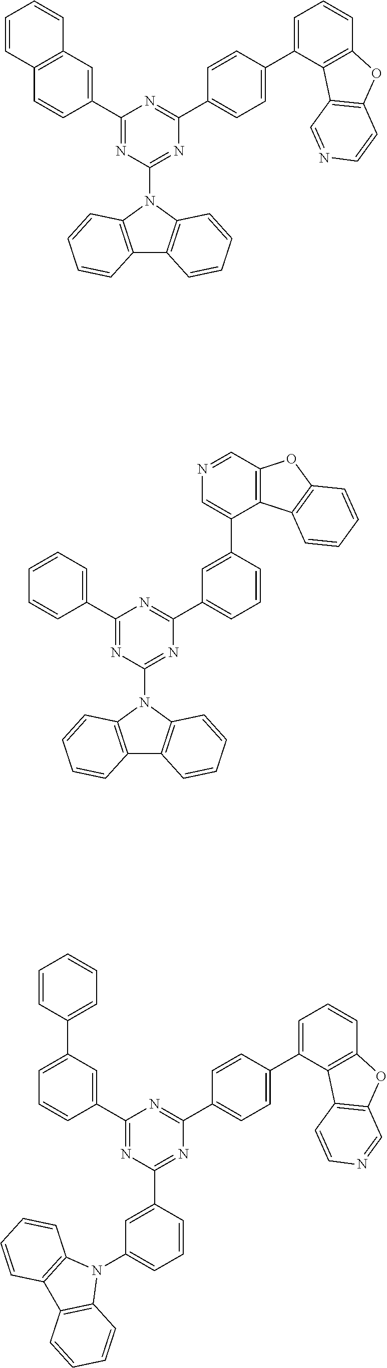 [SMILES: c1ccc2cc(-c3nc(-c4ccc(-c5cccc6oc7ccncc7c56)cc4)nc(-n4c5ccccc5c5ccccc54)n3)ccc2c1 ; c1ccc(-c2nc(-c3cccc(-c4cncc5oc6ccccc6c45)c3)nc(-n3c4ccccc4c4ccccc43)n2)cc1 ; c1ccc(-c2cccc(-c3nc(-c4ccc(-c5cccc6oc7cnccc7c56)cc4)nc(-c4cccc(-n5c6ccccc6c6ccccc65)c4)n3)c2)cc1]

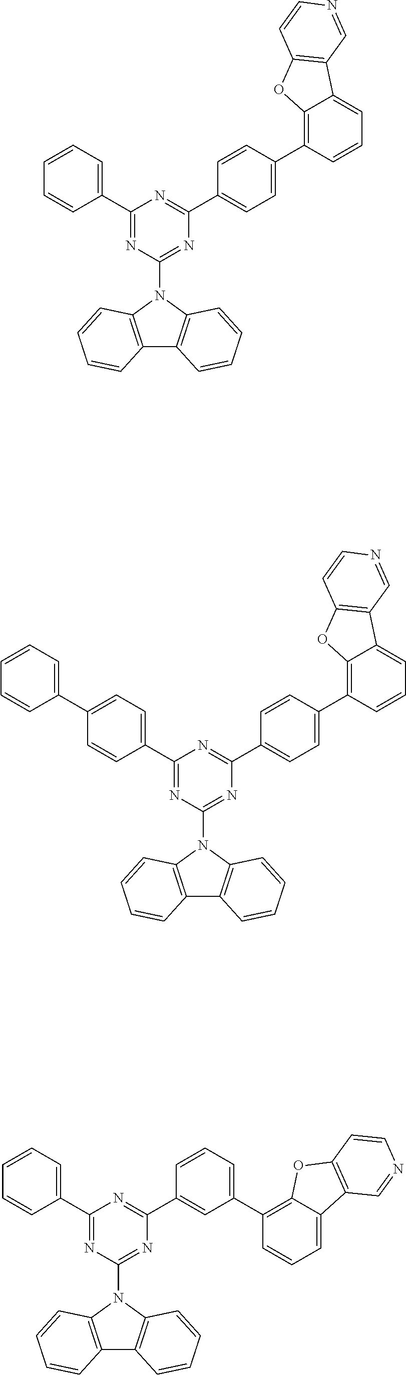 [SMILES: c1ccc(-c2nc(-c3ccc(-c4cccc5c4oc4ccncc45)cc3)nc(-n3c4ccccc4c4ccccc43)n2)cc1 ; c1ccc(-c2ccc(-c3nc(-c4ccc(-c5cccc6c5oc5ccncc56)cc4)nc(-n4c5ccccc5c5ccccc54)n3)cc2)cc1 ; c1ccc(-c2nc(-c3cccc(-c4cccc5c4oc4ccncc45)c3)nc(-n3c4ccccc4c4ccccc43)n2)cc1]

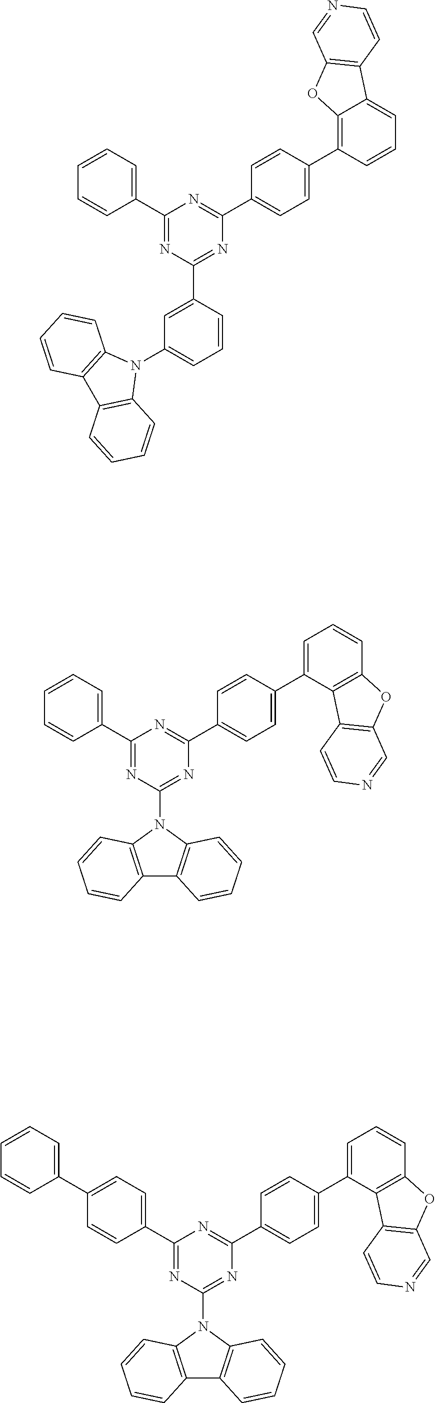 [SMILES: c1ccc(-c2nc(-c3ccc(-c4cccc5c4oc4cnccc45)cc3)nc(-c3cccc(-n4c5ccccc5c5ccccc54)c3)n2)cc1 ; c1ccc(-c2nc(-c3ccc(-c4cccc5oc6cnccc6c45)cc3)nc(-n3c4ccccc4c4ccccc43)n2)cc1 ; c1ccc(-c2ccc(-c3nc(-c4ccc(-c5cccc6oc7cnccc7c56)cc4)nc(-n4c5ccccc5c5ccccc54)n3)cc2)cc1]

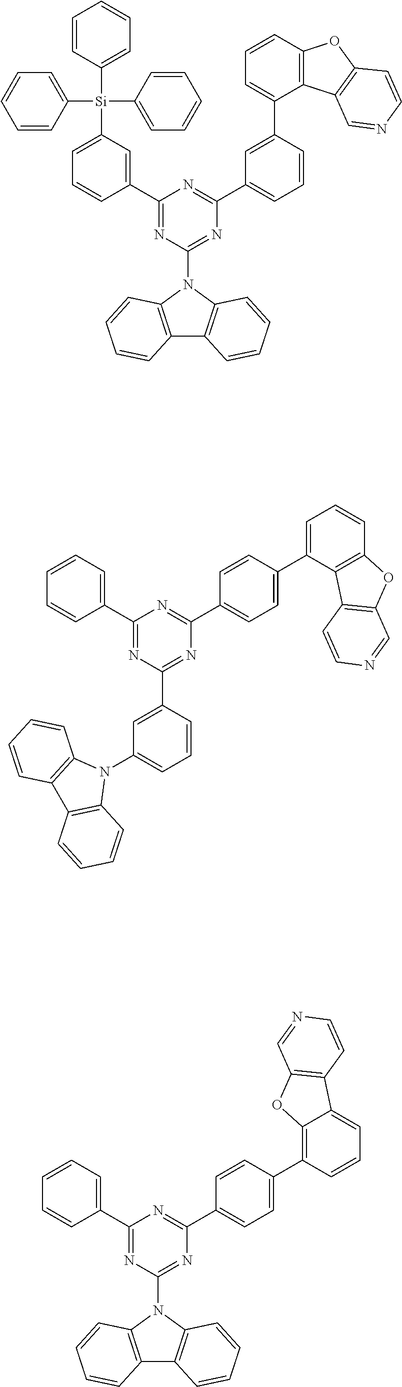 [SMILES: c1ccc([Si](c2ccccc2)(c2ccccc2)c2cccc(-c3nc(-c4cccc(-c5cccc6oc7ccncc7c56)c4)nc(-n4c5ccccc5c5ccccc54)n3)c2)cc1 ; c1ccc(-c2nc(-c3ccc(-c4cccc5oc6cnccc6c45)cc3)nc(-c3cccc(-n4c5ccccc5c5ccccc54)c3)n2)cc1 ; c1ccc(-c2nc(-c3ccc(-c4cccc5c4oc4cnccc45)cc3)nc(-n3c4ccccc4c4ccccc43)n2)cc1]

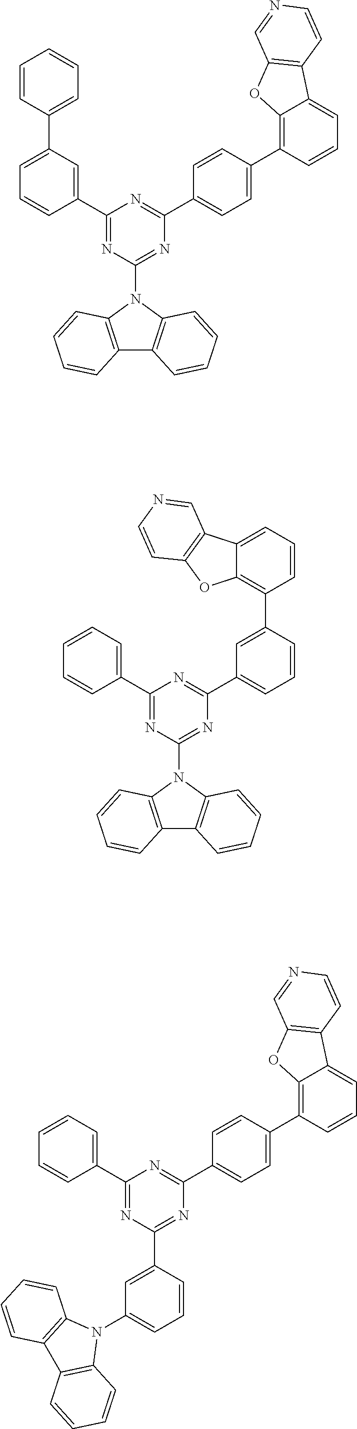 [SMILES: c1ccc(-c2cccc(-c3nc(-c4ccc(-c5cccc6c5oc5cnccc56)cc4)nc(-n4c5ccccc5c5ccccc54)n3)c2)cc1 ; c1ccc(-c2nc(-c3cccc(-c4cccc5c4oc4ccncc45)c3)nc(-n3c4ccccc4c4ccccc43)n2)cc1 ; c1ccc(-c2nc(-c3ccc(-c4cccc5c4oc4cnccc45)cc3)nc(-c3cccc(-n4c5ccccc5c5ccccc54)c3)n2)cc1]

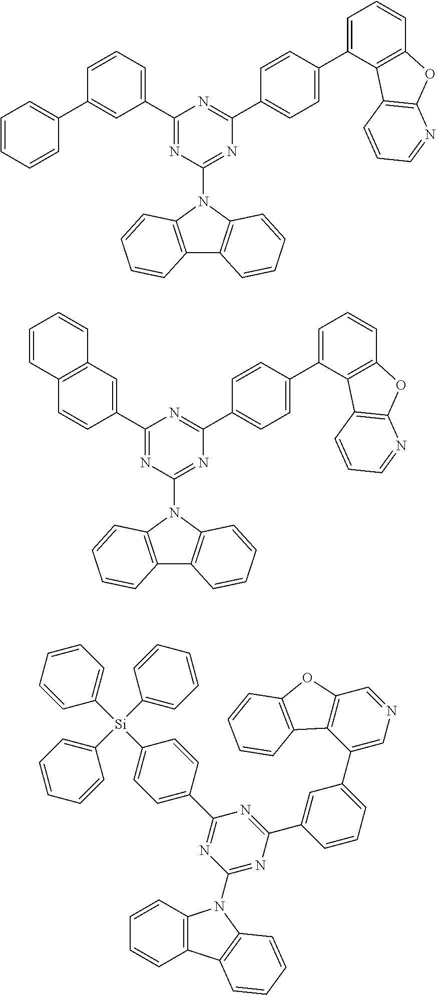 [SMILES: c1ccc(-c2cccc(-c3nc(-c4ccc(-c5cccc6oc7ncccc7c56)cc4)nc(-n4c5ccccc5c5ccccc54)n3)c2)cc1 ; c1ccc2cc(-c3nc(-c4ccc(-c5cccc6oc7ncccc7c56)cc4)nc(-n4c5ccccc5c5ccccc54)n3)ccc2c1 ; c1ccc([Si](c2ccccc2)(c2ccccc2)c2ccc(-c3nc(-c4cccc(-c5cncc6oc7ccccc7c56)c4)nc(-n4c5ccccc5c5ccccc54)n3)cc2)cc1]

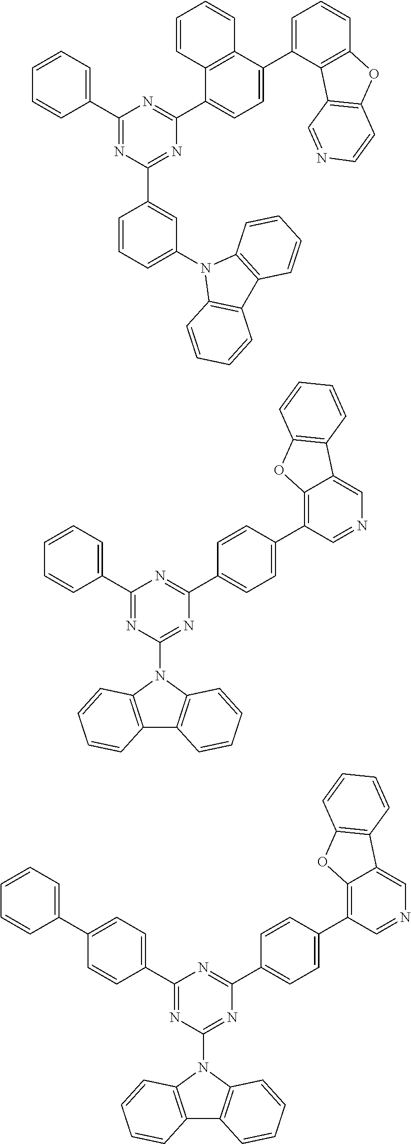 [SMILES: c1ccc(-c2nc(-c3cccc(-n4c5ccccc5c5ccccc54)c3)nc(-c3ccc(-c4cccc5oc6ccncc6c45)c4ccccc34)n2)cc1 ; c1ccc(-c2nc(-c3ccc(-c4cncc5c4oc4ccccc45)cc3)nc(-n3c4ccccc4c4ccccc43)n2)cc1 ; c1ccc(-c2ccc(-c3nc(-c4ccc(-c5cncc6c5oc5ccccc56)cc4)nc(-n4c5ccccc5c5ccccc54)n3)cc2)cc1]

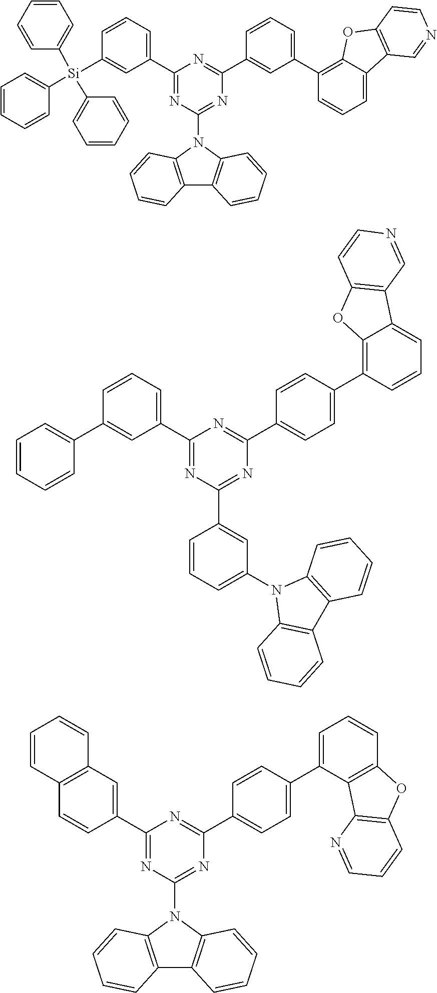 [SMILES: c1ccc([Si](c2ccccc2)(c2ccccc2)c2cccc(-c3nc(-c4cccc(-c5cccc6c5oc5ccncc56)c4)nc(-n4c5ccccc5c5ccccc54)n3)c2)cc1 ; c1ccc(-c2cccc(-c3nc(-c4ccc(-c5cccc6c5oc5ccncc56)cc4)nc(-c4cccc(-n5c6ccccc6c6ccccc65)c4)n3)c2)cc1 ; c1ccc2cc(-c3nc(-c4ccc(-c5cccc6oc7cccnc7c56)cc4)nc(-n4c5ccccc5c5ccccc54)n3)ccc2c1]

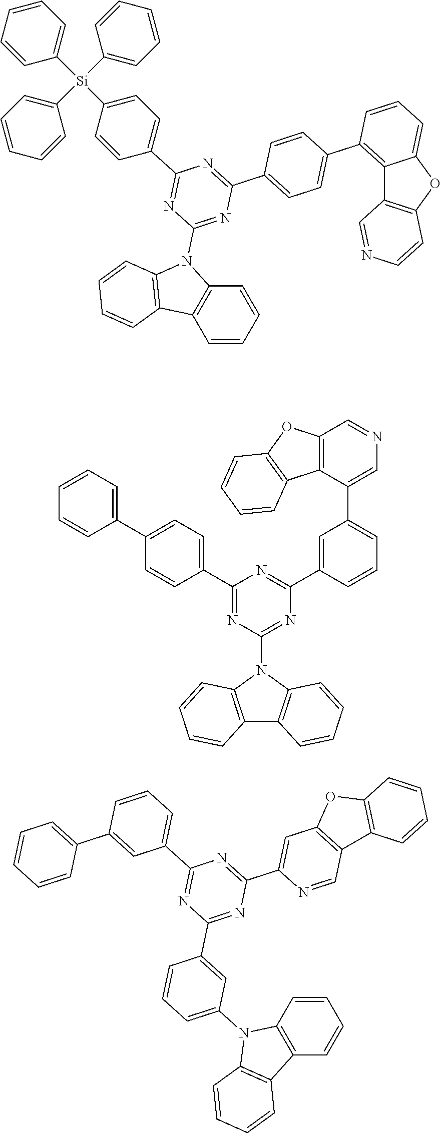 [SMILES: c1ccc([Si](c2ccccc2)(c2ccccc2)c2ccc(-c3nc(-c4ccc(-c5cccc6oc7ccncc7c56)cc4)nc(-n4c5ccccc5c5ccccc54)n3)cc2)cc1 ; c1ccc(-c2ccc(-c3nc(-c4cccc(-c5cncc6oc7ccccc7c56)c4)nc(-n4c5ccccc5c5ccccc54)n3)cc2)cc1 ; c1ccc(-c2cccc(-c3nc(-c4cccc(-n5c6ccccc6c6ccccc65)c4)nc(-c4cc5oc6ccccc6c5cn4)n3)c2)cc1]

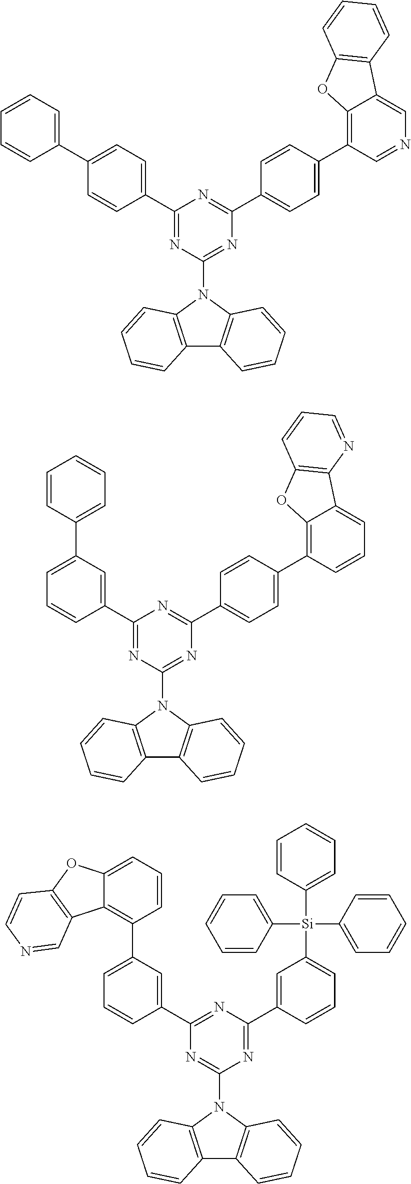 [SMILES: c1ccc(-c2ccc(-c3nc(-c4ccc(-c5cncc6c5oc5ccccc56)cc4)nc(-n4c5ccccc5c5ccccc54)n3)cc2)cc1 ; c1ccc(-c2cccc(-c3nc(-c4ccc(-c5cccc6c5oc5cccnc56)cc4)nc(-n4c5ccccc5c5ccccc54)n3)c2)cc1 ; c1ccc([Si](c2ccccc2)(c2ccccc2)c2cccc(-c3nc(-c4cccc(-c5cccc6oc7ccncc7c56)c4)nc(-n4c5ccccc5c5ccccc54)n3)c2)cc1]

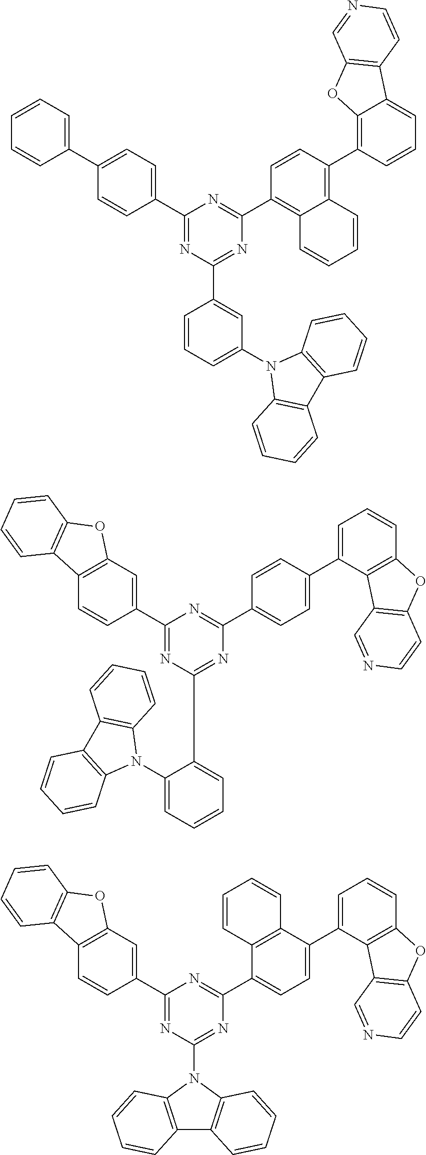 [SMILES: c1ccc(-c2ccc(-c3nc(-c4cccc(-n5c6ccccc6c6ccccc65)c4)nc(-c4ccc(-c5cccc6c5oc5cnccc56)c5ccccc45)n3)cc2)cc1 ; c1ccc(-n2c3ccccc3c3ccccc32)c(-c2nc(-c3ccc(-c4cccc5oc6ccncc6c45)cc3)nc(-c3ccc4c(c3)oc3ccccc34)n2)c1 ; c1ccc2c(c1)oc1cc(-c3nc(-c4ccc(-c5cccc6oc7ccncc7c56)c5ccccc45)nc(-n4c5ccccc5c5ccccc54)n3)ccc12]

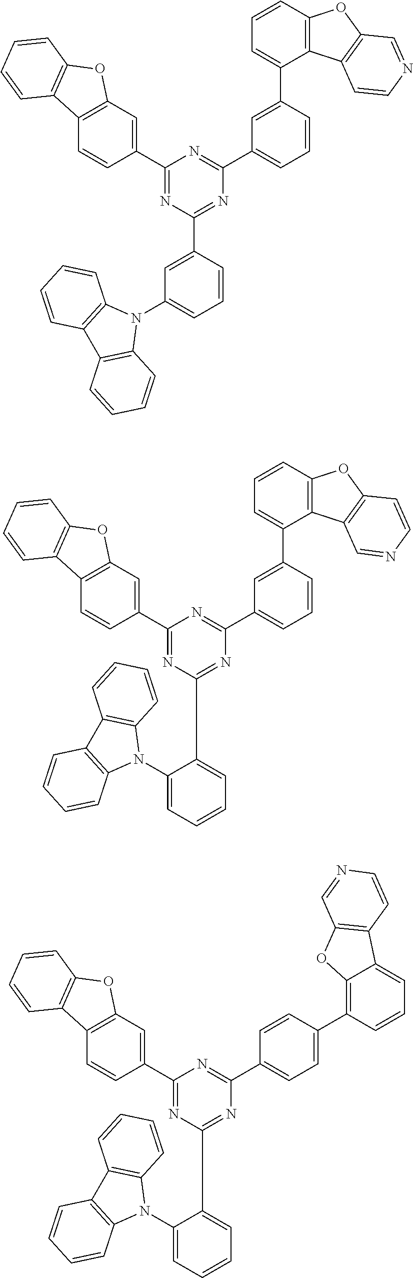 [SMILES: c1cc(-c2nc(-c3cccc(-n4c5ccccc5c5ccccc54)c3)nc(-c3ccc4c(c3)oc3ccccc34)n2)cc(-c2cccc3oc4cnccc4c23)c1 ; c1cc(-c2nc(-c3ccc4c(c3)oc3ccccc34)nc(-c3ccccc3-n3c4ccccc4c4ccccc43)n2)cc(-c2cccc3oc4ccncc4c23)c1 ; c1ccc(-n2c3ccccc3c3ccccc32)c(-c2nc(-c3ccc(-c4cccc5c4oc4cnccc45)cc3)nc(-c3ccc4c(c3)oc3ccccc34)n2)c1]

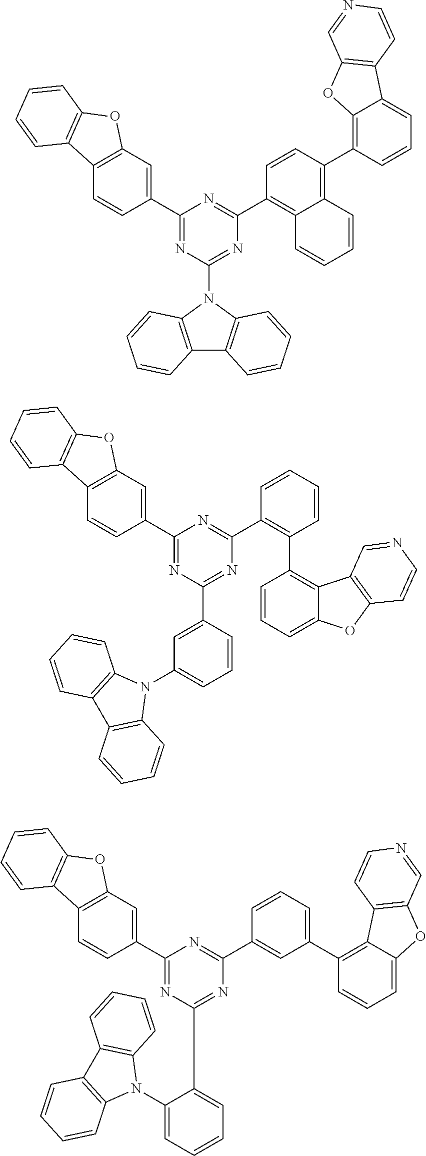 [SMILES: c1ccc2c(c1)oc1cc(-c3nc(-c4ccc(-c5cccc6c5oc5cnccc56)c5ccccc45)nc(-n4c5ccccc5c5ccccc54)n3)ccc12 ; c1cc(-c2nc(-c3ccc4c(c3)oc3ccccc34)nc(-c3ccccc3-c3cccc4oc5ccncc5c34)n2)cc(-n2c3ccccc3c3ccccc32)c1 ; c1cc(-c2nc(-c3ccc4c(c3)oc3ccccc34)nc(-c3ccccc3-n3c4ccccc4c4ccccc43)n2)cc(-c2cccc3oc4cnccc4c23)c1]

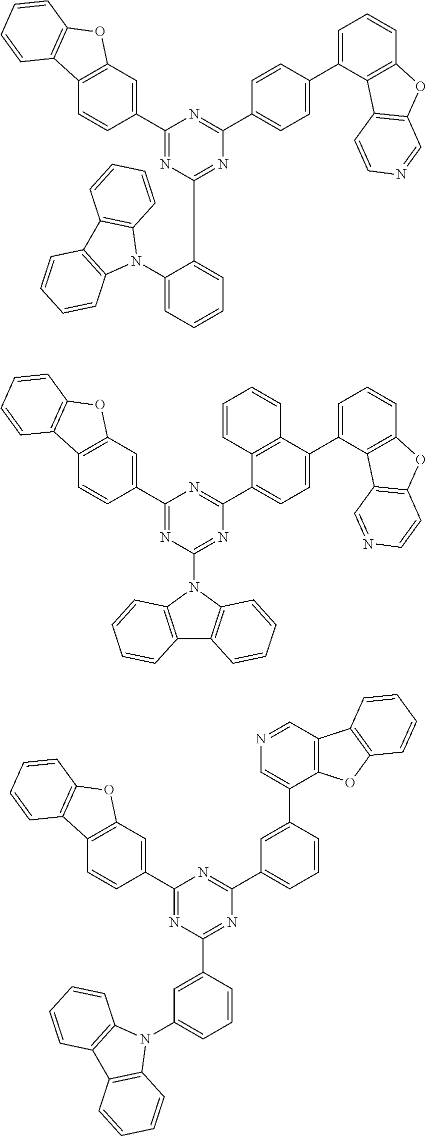 [SMILES: c1ccc(-n2c3ccccc3c3ccccc32)c(-c2nc(-c3ccc(-c4cccc5oc6cnccc6c45)cc3)nc(-c3ccc4c(c3)oc3ccccc34)n2)c1 ; c1ccc2c(c1)oc1cc(-c3nc(-c4ccc(-c5cccc6oc7ccncc7c56)c5ccccc45)nc(-n4c5ccccc5c5ccccc54)n3)ccc12 ; c1cc(-c2nc(-c3cccc(-n4c5ccccc5c5ccccc54)c3)nc(-c3ccc4c(c3)oc3ccccc34)n2)cc(-c2cncc3c2oc2ccccc23)c1]

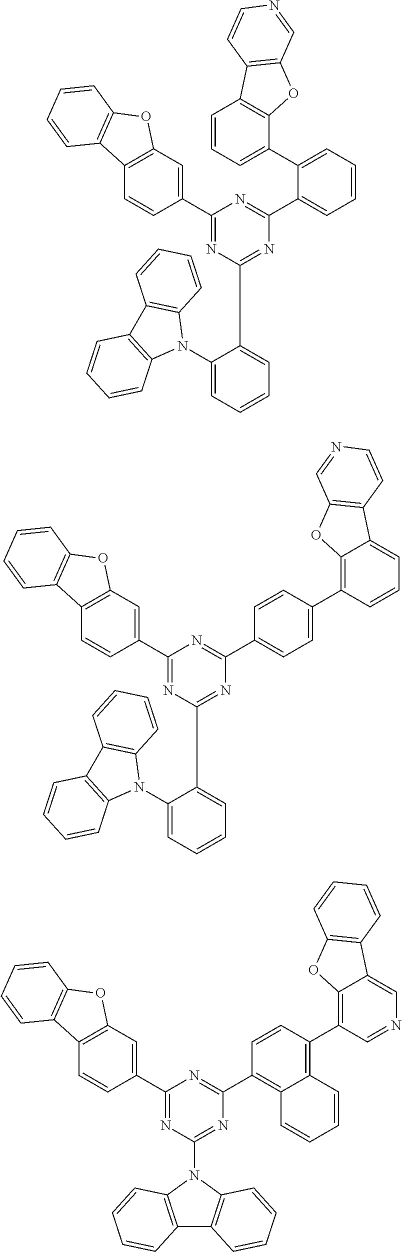 [SMILES: c1ccc(-c2cccc3c2oc2cnccc23)c(-c2nc(-c3ccc4c(c3)oc3ccccc34)nc(-c3ccccc3-n3c4ccccc4c4ccccc43)n2)c1 ; c1ccc(-n2c3ccccc3c3ccccc32)c(-c2nc(-c3ccc(-c4cccc5c4oc4cnccc45)cc3)nc(-c3ccc4c(c3)oc3ccccc34)n2)c1 ; c1ccc2c(c1)oc1cc(-c3nc(-c4ccc(-c5cncc6c5oc5ccccc56)c5ccccc45)nc(-n4c5ccccc5c5ccccc54)n3)ccc12]

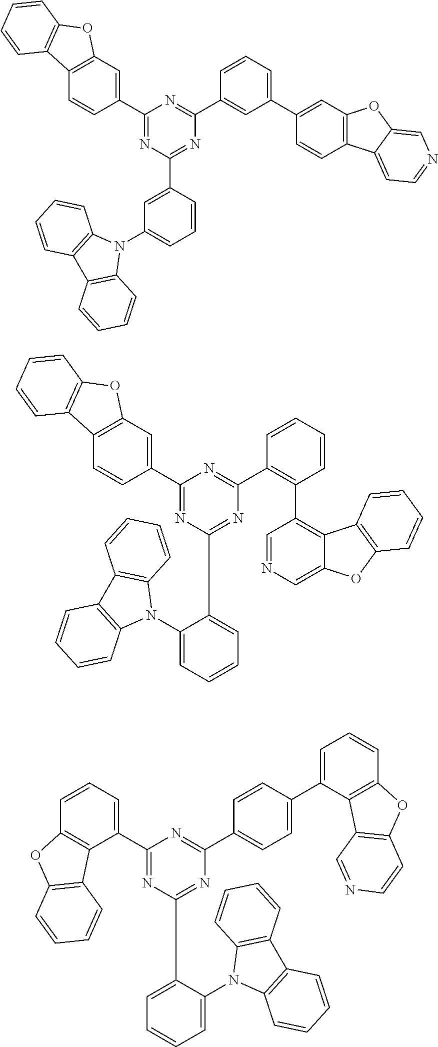

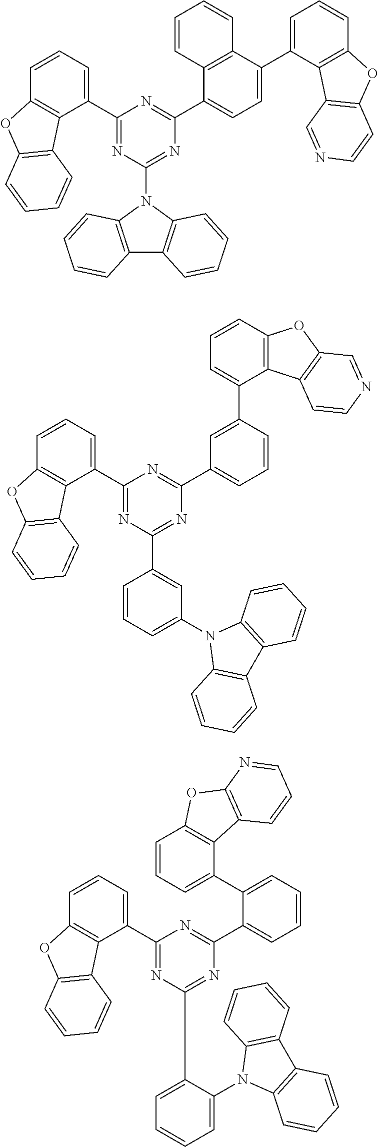

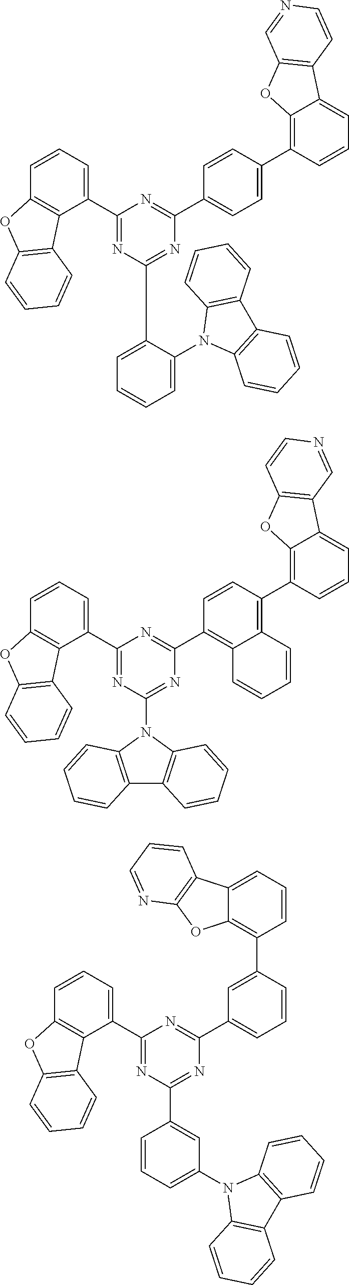

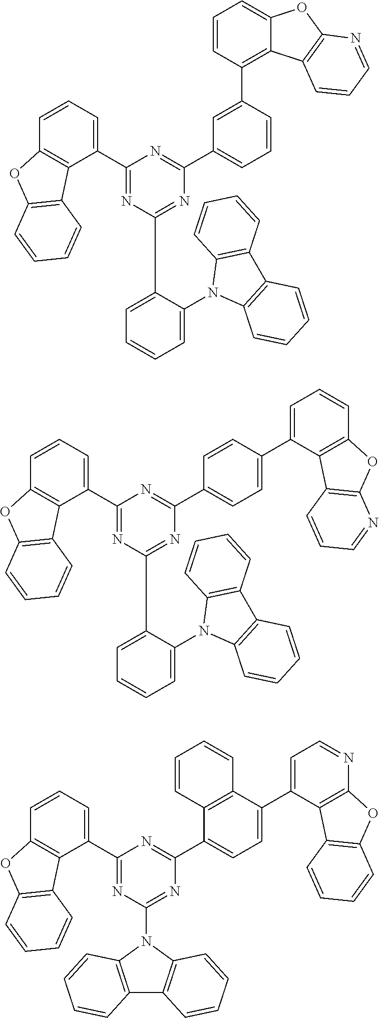 [SMILES: c1cc(-c2nc(-c3ccccc3-n3c4ccccc4c4ccccc43)nc(-c3cccc4oc5ccccc5c34)n2)cc(-c2cccc3oc4ncccc4c23)c1 ; c1ccc(-n2c3ccccc3c3ccccc32)c(-c2nc(-c3ccc(-c4cccc5oc6ncccc6c45)cc3)nc(-c3cccc4oc5ccccc5c34)n2)c1 ; c1ccc2c(c1)oc1cccc(-c3nc(-c4ccc(-c5ccnc6oc7ccccc7c56)c5ccccc45)nc(-n4c5ccccc5c5ccccc54)n3)c12]

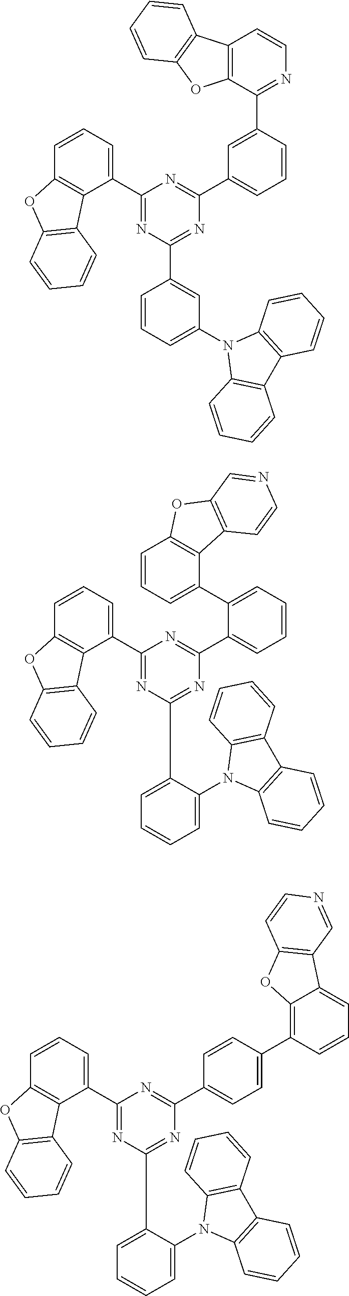

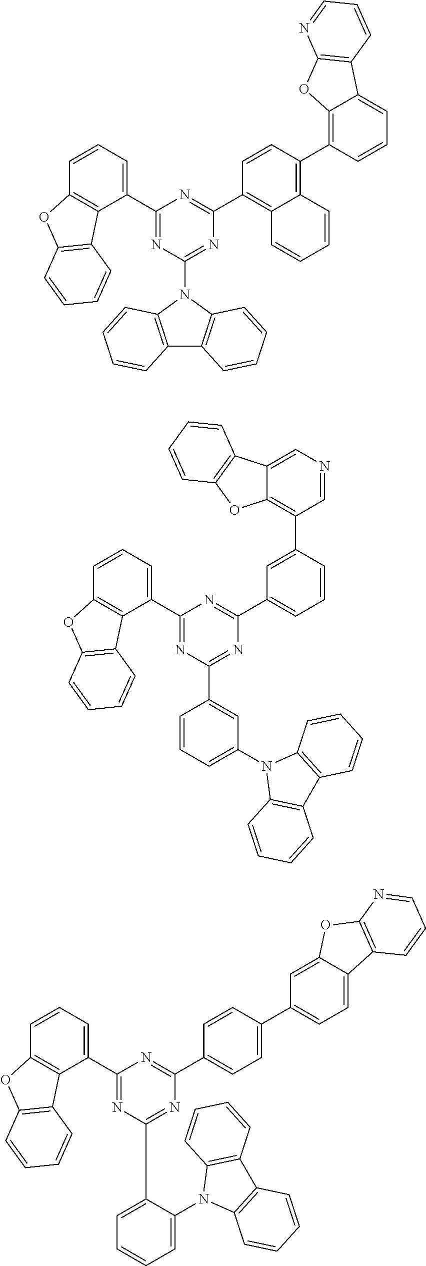 [SMILES: c1ccc2c(c1)oc1cccc(-c3nc(-c4ccc(-c5cccc6c5oc5ncccc56)c5ccccc45)nc(-n4c5ccccc5c5ccccc54)n3)c12 ; c1cc(-c2nc(-c3cccc(-n4c5ccccc5c5ccccc54)c3)nc(-c3cccc4oc5ccccc5c34)n2)cc(-c2cncc3c2oc2ccccc23)c1 ; c1ccc(-n2c3ccccc3c3ccccc32)c(-c2nc(-c3ccc(-c4ccc5c(c4)oc4ncccc45)cc3)nc(-c3cccc4oc5ccccc5c34)n2)c1]

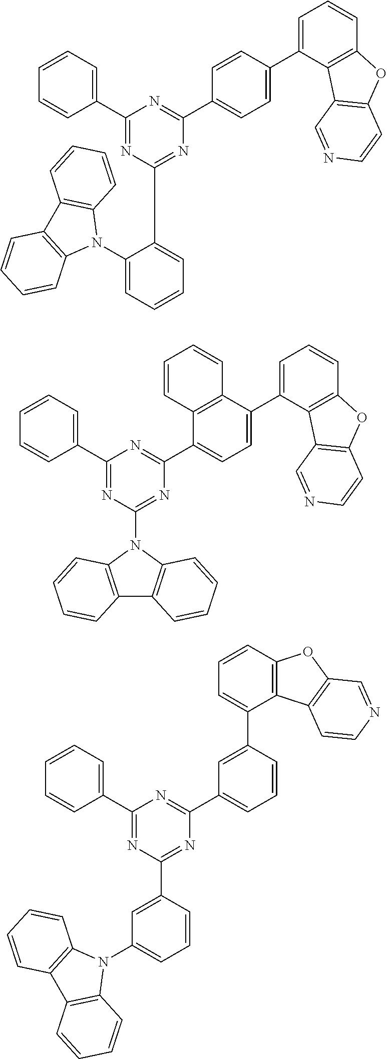 [SMILES: c1ccc(-c2nc(-c3ccc(-c4cccc5oc6ccncc6c45)cc3)nc(-c3ccccc3-n3c4ccccc4c4ccccc43)n2)cc1 ; c1ccc(-c2nc(-c3ccc(-c4cccc5oc6ccncc6c45)c4ccccc34)nc(-n3c4ccccc4c4ccccc43)n2)cc1 ; c1ccc(-c2nc(-c3cccc(-c4cccc5oc6cnccc6c45)c3)nc(-c3cccc(-n4c5ccccc5c5ccccc54)c3)n2)cc1]

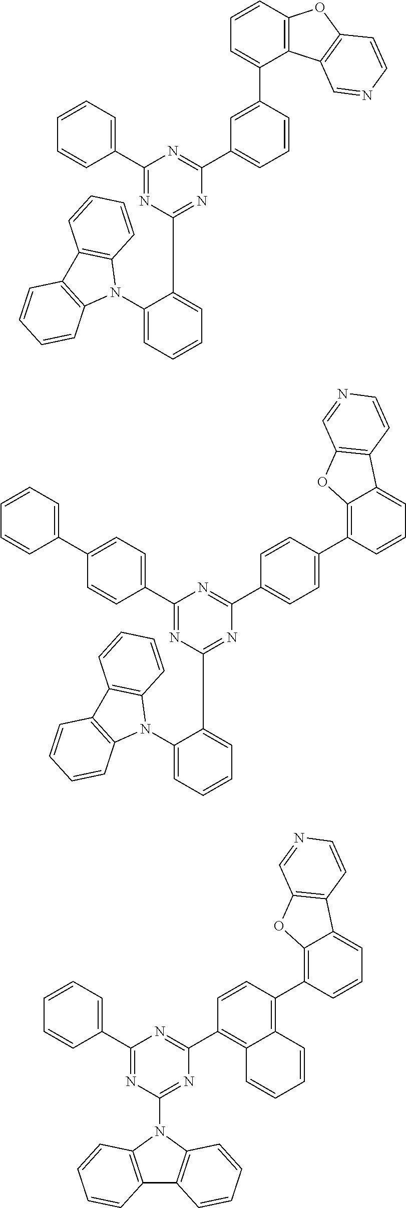 [SMILES: c1ccc(-c2nc(-c3cccc(-c4cccc5oc6ccncc6c45)c3)nc(-c3ccccc3-n3c4ccccc4c4ccccc43)n2)cc1 ; c1ccc(-c2ccc(-c3nc(-c4ccc(-c5cccc6c5oc5cnccc56)cc4)nc(-c4ccccc4-n4c5ccccc5c5ccccc54)n3)cc2)cc1 ; c1ccc(-c2nc(-c3ccc(-c4cccc5c4oc4cnccc45)c4ccccc34)nc(-n3c4ccccc4c4ccccc43)n2)cc1]

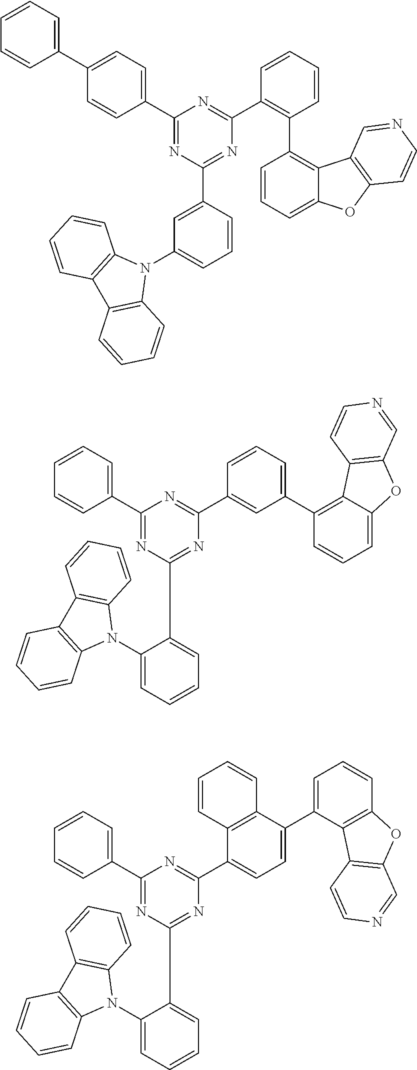 [SMILES: c1ccc(-c2ccc(-c3nc(-c4cccc(-n5c6ccccc6c6ccccc65)c4)nc(-c4ccccc4-c4cccc5oc6ccncc6c45)n3)cc2)cc1 ; c1ccc(-c2nc(-c3cccc(-c4cccc5oc6cnccc6c45)c3)nc(-c3ccccc3-n3c4ccccc4c4ccccc43)n2)cc1 ; c1ccc(-c2nc(-c3ccccc3-n3c4ccccc4c4ccccc43)nc(-c3ccc(-c4cccc5oc6cnccc6c45)c4ccccc34)n2)cc1]

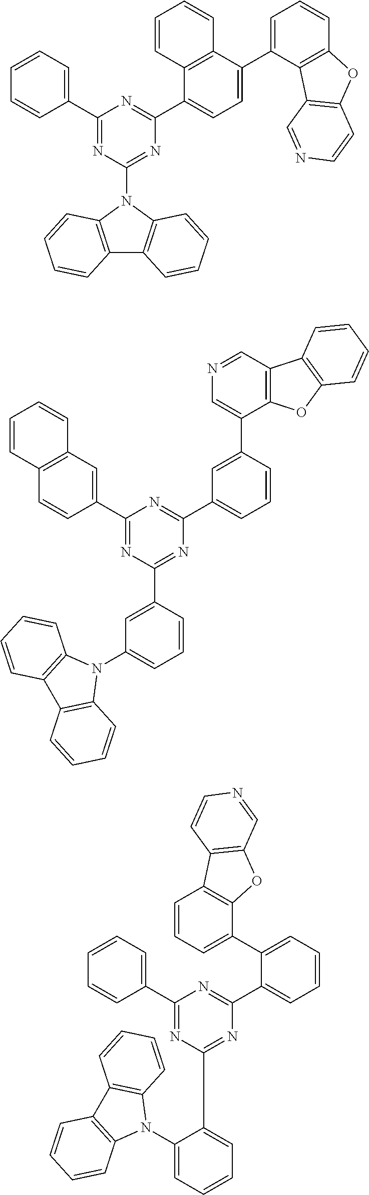 [SMILES: c1ccc(-c2nc(-c3ccc(-c4cccc5oc6ccncc6c45)c4ccccc34)nc(-n3c4ccccc4c4ccccc43)n2)cc1 ; c1cc(-c2nc(-c3cccc(-n4c5ccccc5c5ccccc54)c3)nc(-c3ccc4ccccc4c3)n2)cc(-c2cncc3c2oc2ccccc23)c1 ; c1ccc(-c2nc(-c3ccccc3-c3cccc4c3oc3cnccc34)nc(-c3ccccc3-n3c4ccccc4c4ccccc43)n2)cc1]

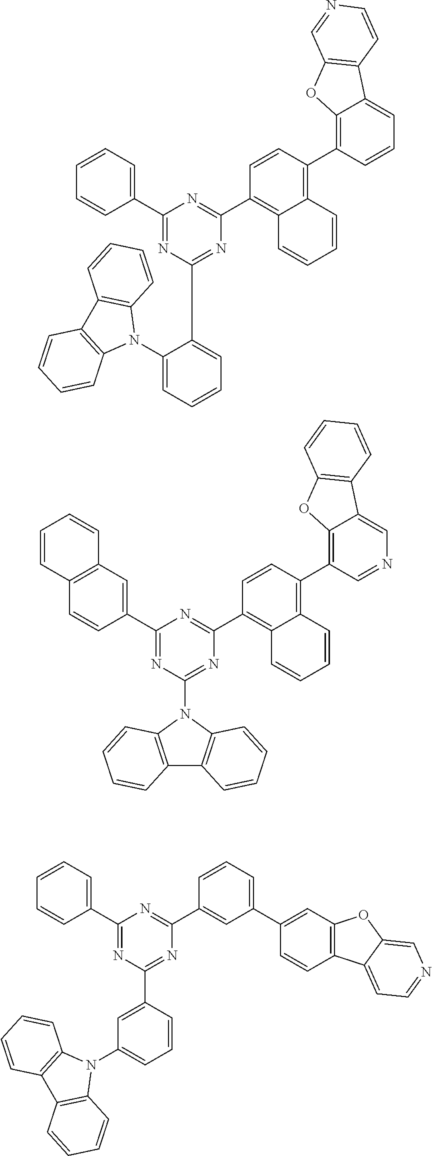 [SMILES: c1ccc(-c2nc(-c3ccccc3-n3c4ccccc4c4ccccc43)nc(-c3ccc(-c4cccc5c4oc4cnccc45)c4ccccc34)n2)cc1 ; c1ccc2cc(-c3nc(-c4ccc(-c5cncc6c5oc5ccccc56)c5ccccc45)nc(-n4c5ccccc5c5ccccc54)n3)ccc2c1 ; c1ccc(-c2nc(-c3cccc(-c4ccc5c(c4)oc4cnccc45)c3)nc(-c3cccc(-n4c5ccccc5c5ccccc54)c3)n2)cc1]

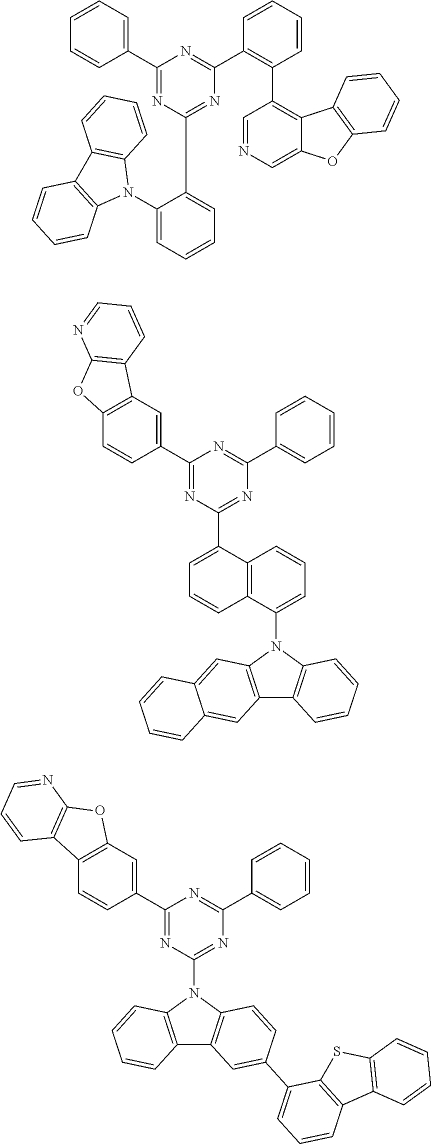 [SMILES: c1ccc(-c2nc(-c3ccccc3-c3cncc4oc5ccccc5c34)nc(-c3ccccc3-n3c4ccccc4c4ccccc43)n2)cc1 ; c1ccc(-c2nc(-c3ccc4oc5ncccc5c4c3)nc(-c3cccc4c(-n5c6ccccc6c6cc7ccccc7cc65)cccc34)n2)cc1 ; c1ccc(-c2nc(-c3ccc4c(c3)oc3ncccc34)nc(-n3c4ccccc4c4cc(-c5cccc6c5sc5ccccc56)ccc43)n2)cc1]

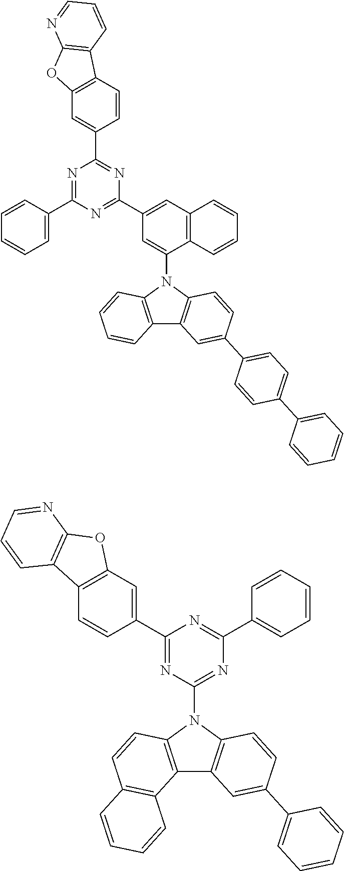 [SMILES: c1ccc(-c2ccc(-c3ccc4c(c3)c3ccccc3n4-c3cc(-c4nc(-c5ccccc5)nc(-c5ccc6c(c5)oc5ncccc56)n4)cc4ccccc34)cc2)cc1 ; c1ccc(-c2ccc3c(c2)c2c4ccccc4ccc2n3-c2nc(-c3ccccc3)nc(-c3ccc4c(c3)oc3ncccc34)n2)cc1]

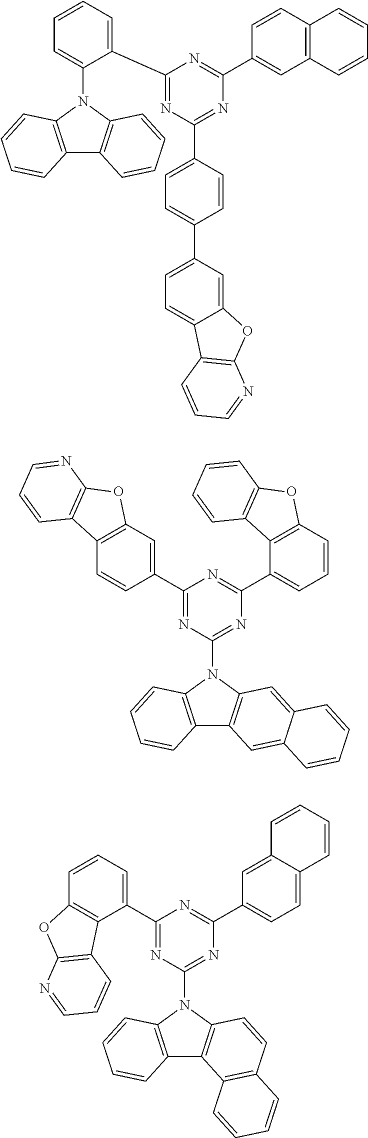

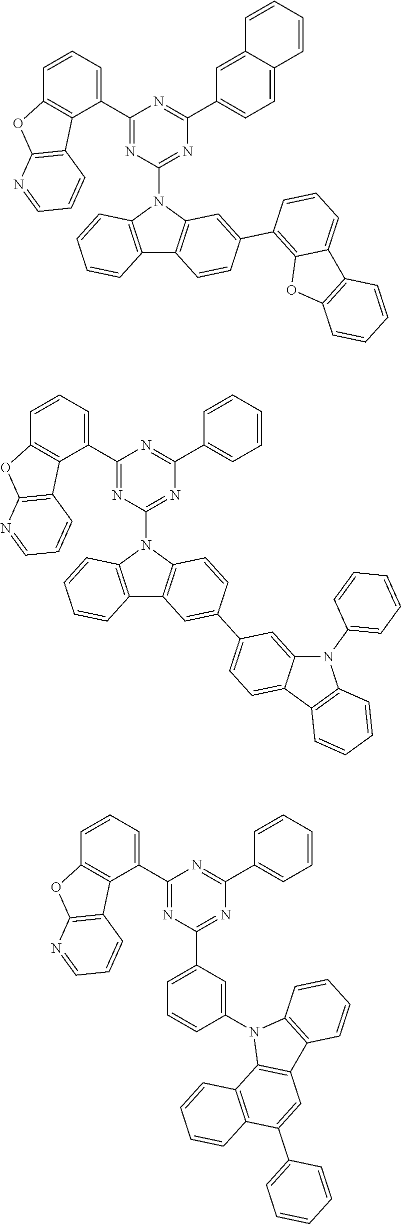 [SMILES: c1ccc2cc(-c3nc(-c4cccc5oc6ncccc6c45)nc(-n4c5ccccc5c5ccc(-c6cccc7c6oc6ccccc67)cc54)n3)ccc2c1 ; c1ccc(-c2nc(-c3cccc4oc5ncccc5c34)nc(-n3c4ccccc4c4cc(-c5ccc6c7ccccc7n(-c7ccccc7)c6c5)ccc43)n2)cc1 ; c1ccc(-c2nc(-c3cccc(-n4c5ccccc5c5cc(-c6ccccc6)c6ccccc6c54)c3)nc(-c3cccc4oc5ncccc5c34)n2)cc1]

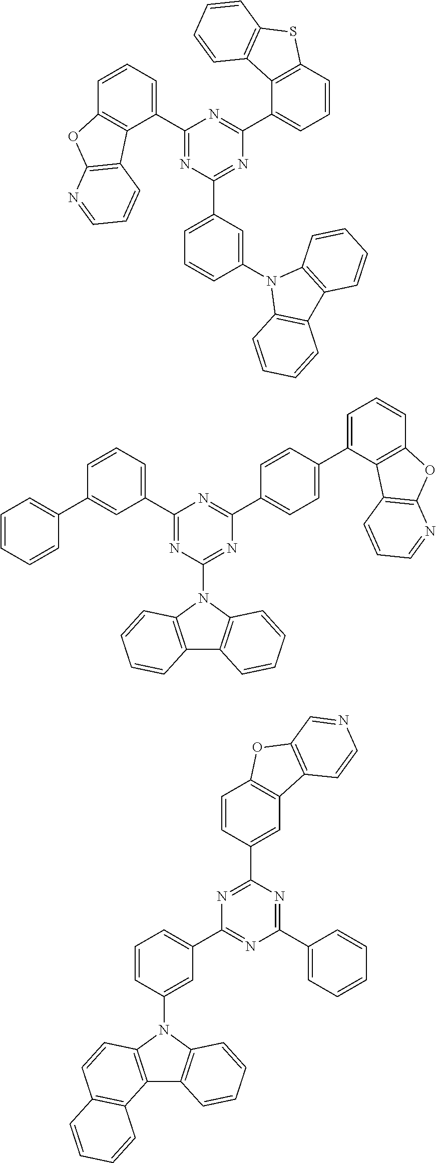 [SMILES: c1cc(-c2nc(-c3cccc4oc5ncccc5c34)nc(-c3cccc4sc5ccccc5c34)n2)cc(-n2c3ccccc3c3ccccc32)c1 ; c1ccc(-c2cccc(-c3nc(-c4ccc(-c5cccc6oc7ncccc7c56)cc4)nc(-n4c5ccccc5c5ccccc54)n3)c2)cc1 ; c1ccc(-c2nc(-c3cccc(-n4c5ccccc5c5c6ccccc6ccc54)c3)nc(-c3ccc4oc5cnccc5c4c3)n2)cc1]

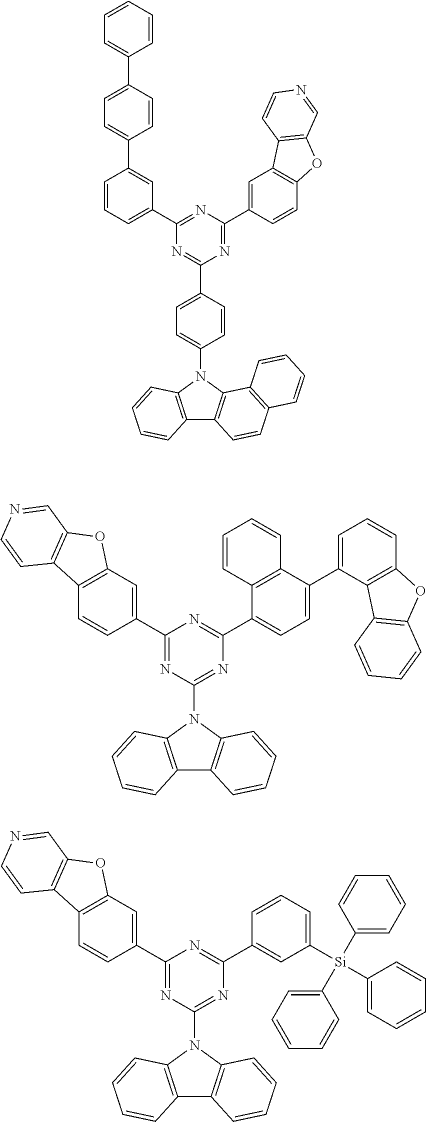 [SMILES: c1ccc(-c2ccc(-c3cccc(-c4nc(-c5ccc(-n6c7ccccc7c7ccc8ccccc8c76)cc5)nc(-c5ccc6oc7cnccc7c6c5)n4)c3)cc2)cc1 ; c1ccc2c(c1)oc1cccc(-c3ccc(-c4nc(-c5ccc6c(c5)oc5cnccc56)nc(-n5c6ccccc6c6ccccc65)n4)c4ccccc34)c12 ; c1ccc([Si](c2ccccc2)(c2ccccc2)c2cccc(-c3nc(-c4ccc5c(c4)oc4cnccc45)nc(-n4c5ccccc5c5ccccc54)n3)c2)cc1]

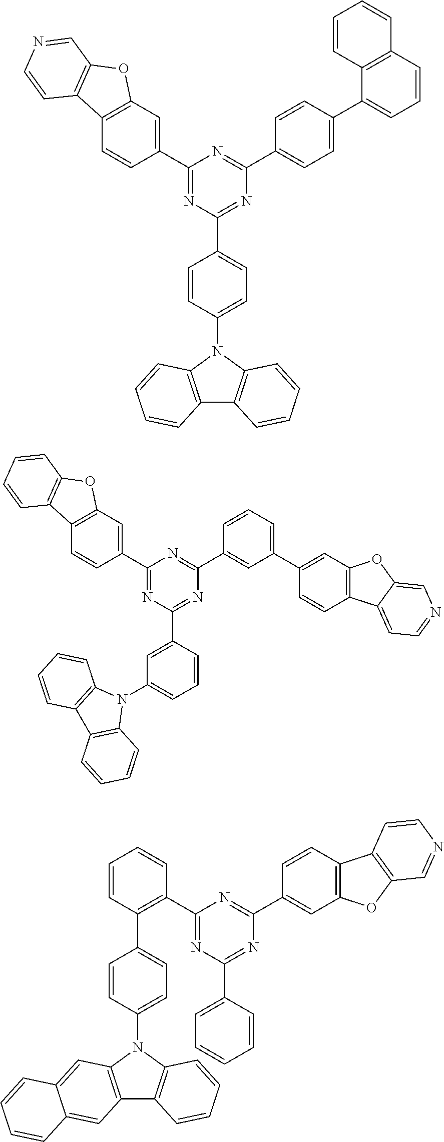 [SMILES: c1ccc2c(-c3ccc(-c4nc(-c5ccc(-n6c7ccccc7c7ccccc76)cc5)nc(-c5ccc6c(c5)oc5cnccc56)n4)cc3)cccc2c1 ; c1cc(-c2ccc3c(c2)oc2cnccc23)cc(-c2nc(-c3cccc(-n4c5ccccc5c5ccccc54)c3)nc(-c3ccc4c(c3)oc3ccccc34)n2)c1 ; c1ccc(-c2nc(-c3ccc4c(c3)oc3cnccc34)nc(-c3ccccc3-c3ccc(-n4c5ccccc5c5cc6ccccc6cc54)cc3)n2)cc1]

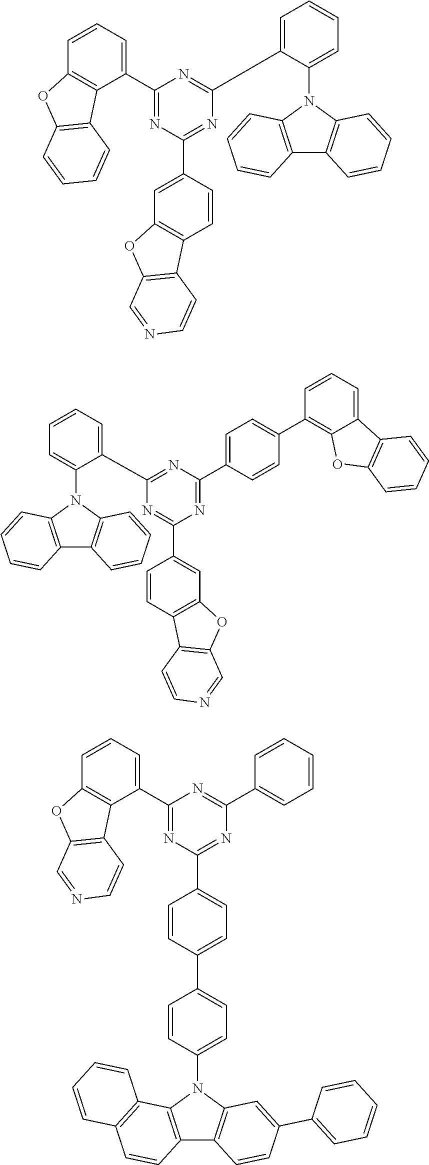 [SMILES: c1ccc(-n2c3ccccc3c3ccccc32)c(-c2nc(-c3ccc4c(c3)oc3cnccc34)nc(-c3cccc4oc5ccccc5c34)n2)c1 ; c1ccc(-n2c3ccccc3c3ccccc32)c(-c2nc(-c3ccc(-c4cccc5c4oc4ccccc45)cc3)nc(-c3ccc4c(c3)oc3cnccc34)n2)c1 ; c1ccc(-c2ccc3c4ccc5ccccc5c4n(-c4ccc(-c5ccc(-c6nc(-c7ccccc7)nc(-c7cccc8oc9cnccc9c78)n6)cc5)cc4)c3c2)cc1]

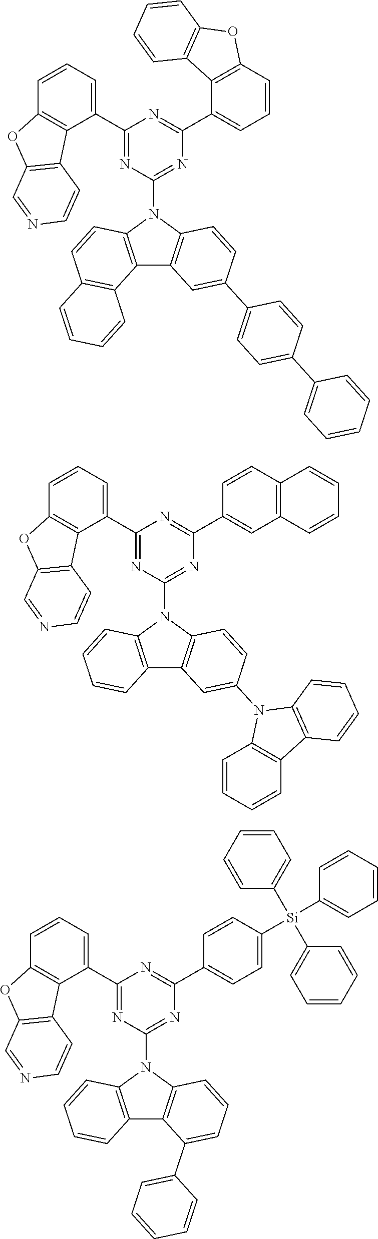

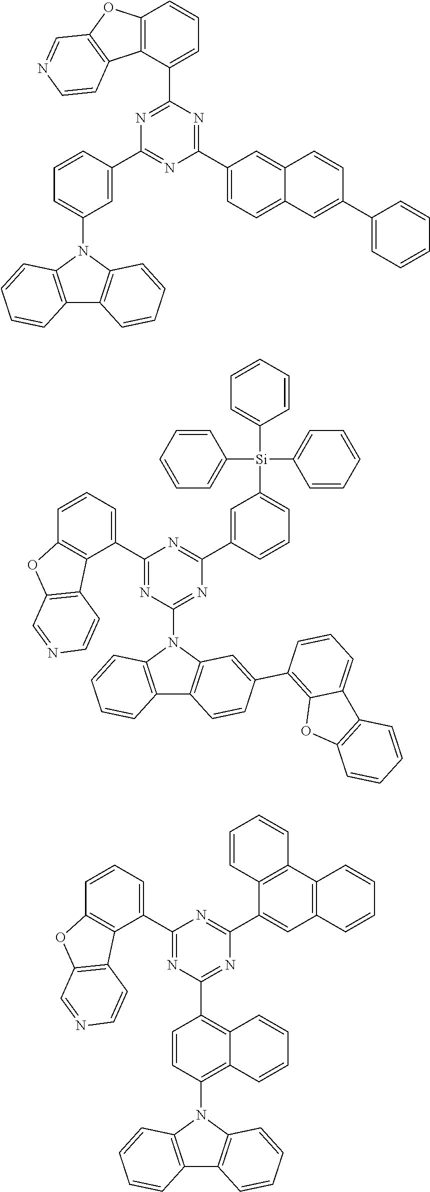 [SMILES: c1ccc(-c2ccc3cc(-c4nc(-c5cccc(-n6c7ccccc7c7ccccc76)c5)nc(-c5cccc6oc7cnccc7c56)n4)ccc3c2)cc1 ; c1ccc([Si](c2ccccc2)(c2ccccc2)c2cccc(-c3nc(-c4cccc5oc6cnccc6c45)nc(-n4c5ccccc5c5ccc(-c6cccc7c6oc6ccccc67)cc54)n3)c2)cc1 ; c1ccc2c(c1)cc(-c1nc(-c3ccc(-n4c5ccccc5c5ccccc54)c4ccccc34)nc(-c3cccc4oc5cnccc5c34)n1)c1ccccc12]

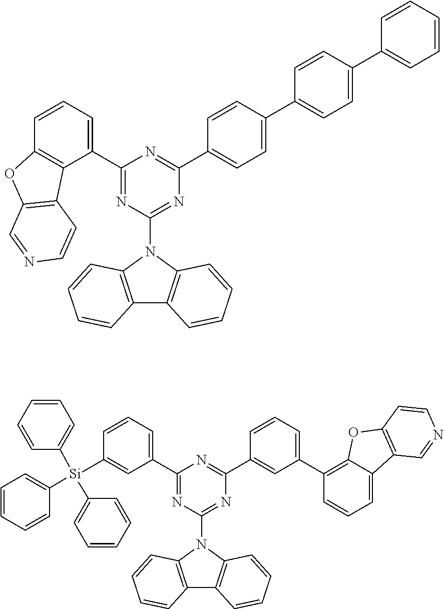 [SMILES: c1ccc(-c2ccc(-c3ccc(-c4nc(-c5cccc6oc7cnccc7c56)nc(-n5c6ccccc6c6ccccc65)n4)cc3)cc2)cc1 ; c1ccc([Si](c2ccccc2)(c2ccccc2)c2cccc(-c3nc(-c4cccc(-c5cccc6c5oc5ccncc56)c4)nc(-n4c5ccccc5c5ccccc54)n3)c2)cc1]

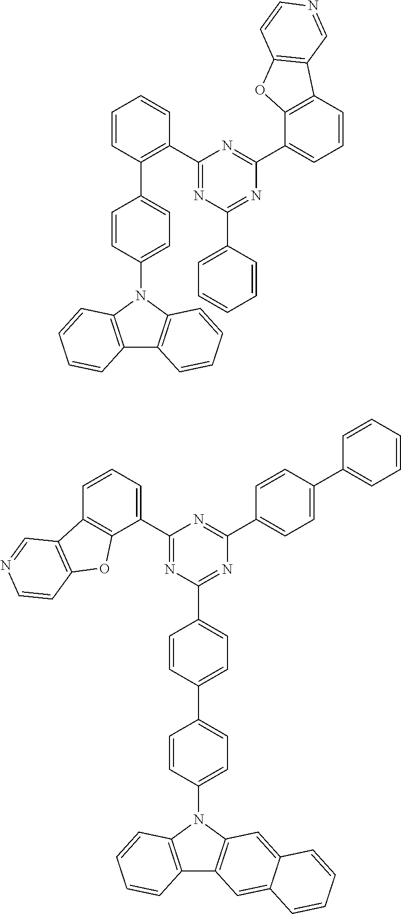 [SMILES: c1ccc(-c2nc(-c3ccccc3-c3ccc(-n4c5ccccc5c5ccccc54)cc3)nc(-c3cccc4c3oc3ccncc34)n2)cc1 ; c1ccc(-c2ccc(-c3nc(-c4ccc(-c5ccc(-n6c7ccccc7c7cc8ccccc8cc76)cc5)cc4)nc(-c4cccc5c4oc4ccncc45)n3)cc2)cc1]

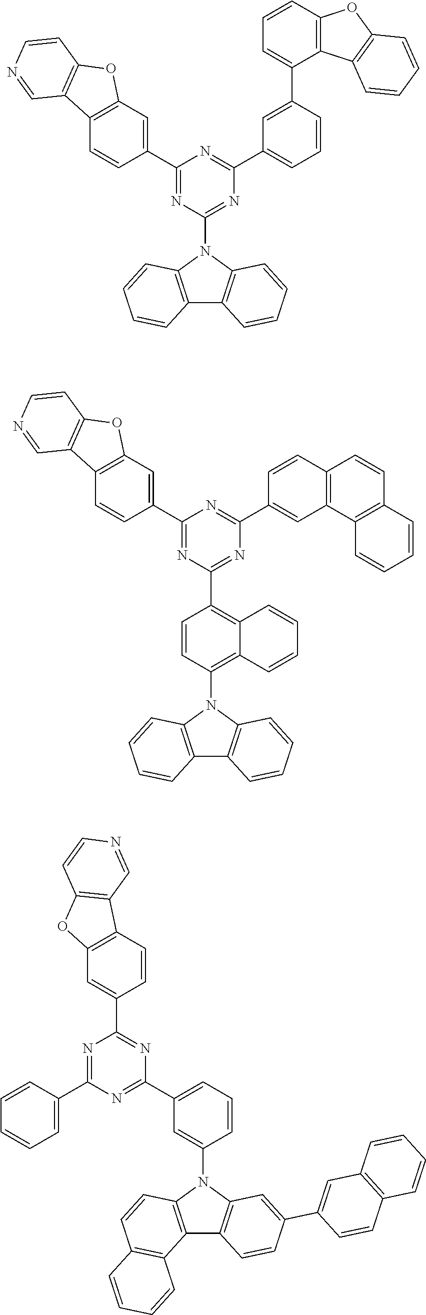 [SMILES: c1cc(-c2nc(-c3ccc4c(c3)oc3ccncc34)nc(-n3c4ccccc4c4ccccc43)n2)cc(-c2cccc3oc4ccccc4c23)c1 ; c1ccc2c(c1)ccc1ccc(-c3nc(-c4ccc5c(c4)oc4ccncc45)nc(-c4ccc(-n5c6ccccc6c6ccccc65)c5ccccc45)n3)cc12 ; c1ccc(-c2nc(-c3cccc(-n4c5cc(-c6ccc7ccccc7c6)ccc5c5c6ccccc6ccc54)c3)nc(-c3ccc4c(c3)oc3ccncc34)n2)cc1]

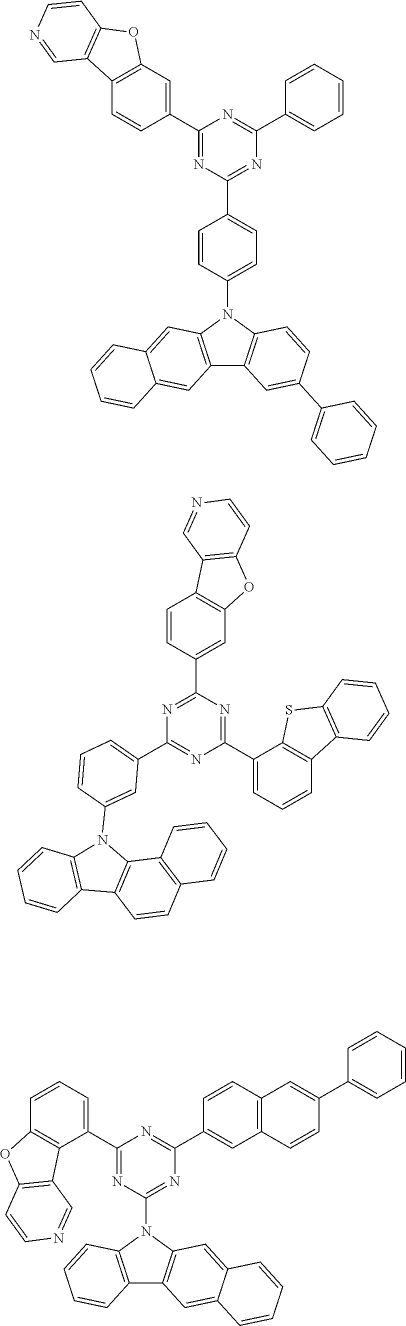 [SMILES: c1ccc(-c2ccc3c(c2)c2cc4ccccc4cc2n3-c2ccc(-c3nc(-c4ccccc4)nc(-c4ccc5c(c4)oc4ccncc45)n3)cc2)cc1 ; c1cc(-c2nc(-c3ccc4c(c3)oc3ccncc34)nc(-c3cccc4c3sc3ccccc34)n2)cc(-n2c3ccccc3c3ccc4ccccc4c32)c1 ; c1ccc(-c2ccc3cc(-c4nc(-c5cccc6oc7ccncc7c56)nc(-n5c6ccccc6c6cc7ccccc7cc65)n4)ccc3c2)cc1]

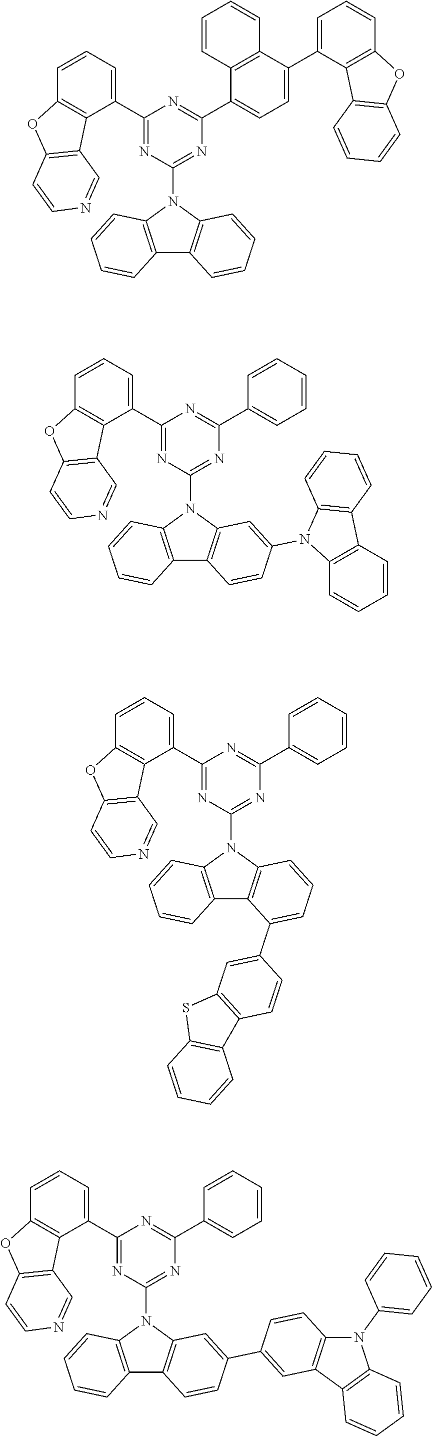 [SMILES: c1ccc2c(c1)oc1cccc(-c3ccc(-c4nc(-c5cccc6oc7ccncc7c56)nc(-n5c6ccccc6c6ccccc65)n4)c4ccccc34)c12 ; c1ccc(-c2nc(-c3cccc4oc5ccncc5c34)nc(-n3c4ccccc4c4ccc(-n5c6ccccc6c6ccccc65)cc43)n2)cc1 ; c1ccc(-c2nc(-c3cccc4oc5ccncc5c34)nc(-n3c4ccccc4c4c(-c5ccc6c(c5)sc5ccccc56)cccc43)n2)cc1 ; c1ccc(-c2nc(-c3cccc4oc5ccncc5c34)nc(-n3c4ccccc4c4ccc(-c5ccc6c(c5)c5ccccc5n6-c5ccccc5)cc43)n2)cc1]

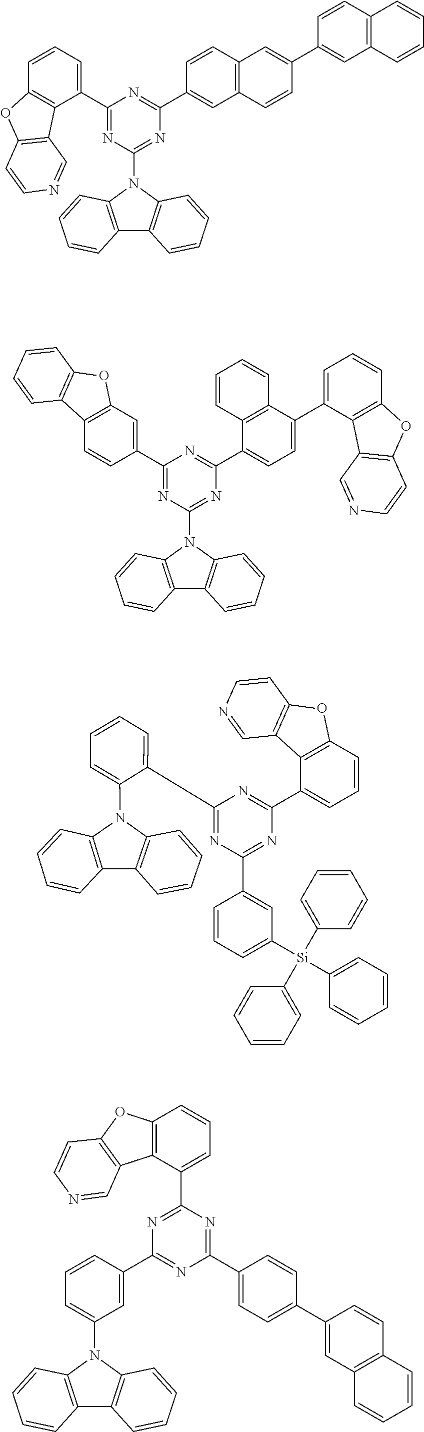 [SMILES: c1ccc2cc(-c3ccc4cc(-c5nc(-c6cccc7oc8ccncc8c67)nc(-n6c7ccccc7c7ccccc76)n5)ccc4c3)ccc2c1 ; c1ccc2c(c1)oc1cc(-c3nc(-c4ccc(-c5cccc6oc7ccncc7c56)c5ccccc45)nc(-n4c5ccccc5c5ccccc54)n3)ccc12 ; c1ccc([Si](c2ccccc2)(c2ccccc2)c2cccc(-c3nc(-c4ccccc4-n4c5ccccc5c5ccccc54)nc(-c4cccc5oc6ccncc6c45)n3)c2)cc1 ; c1cc(-c2nc(-c3ccc(-c4ccc5ccccc5c4)cc3)nc(-c3cccc4oc5ccncc5c34)n2)cc(-n2c3ccccc3c3ccccc32)c1]

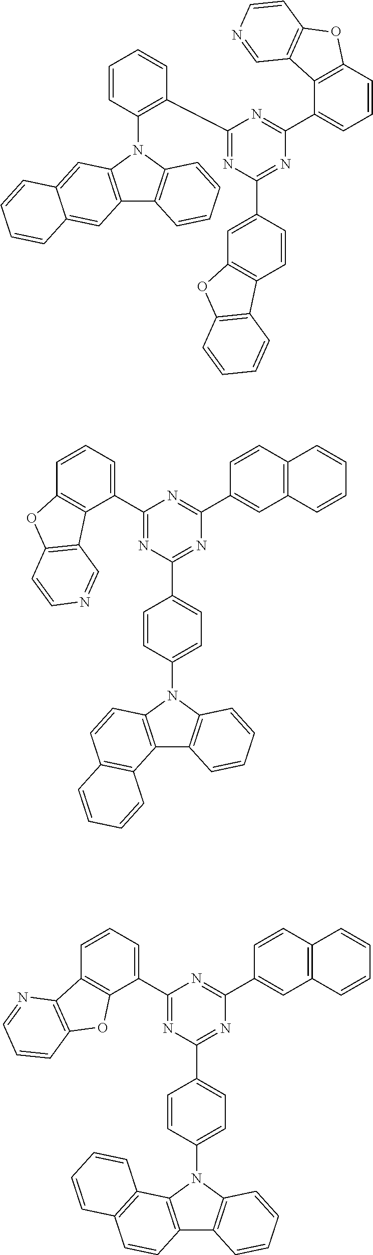 [SMILES: c1ccc(-n2c3ccccc3c3cc4ccccc4cc32)c(-c2nc(-c3ccc4c(c3)oc3ccccc34)nc(-c3cccc4oc5ccncc5c34)n2)c1 ; c1ccc2cc(-c3nc(-c4ccc(-n5c6ccccc6c6c7ccccc7ccc65)cc4)nc(-c4cccc5oc6ccncc6c45)n3)ccc2c1 ; c1ccc2cc(-c3nc(-c4ccc(-n5c6ccccc6c6ccc7ccccc7c65)cc4)nc(-c4cccc5c4oc4cccnc45)n3)ccc2c1]

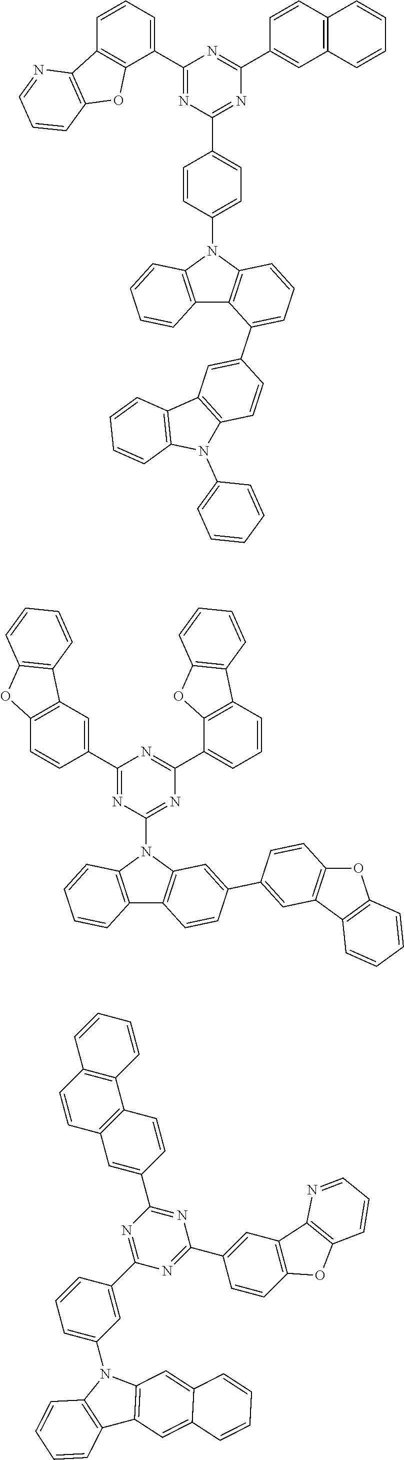 [SMILES: c1ccc(-n2c3ccccc3c3cc(-c4cccc5c4c4ccccc4n5-c4ccc(-c5nc(-c6ccc7ccccc7c6)nc(-c6cccc7c6oc6cccnc67)n5)cc4)ccc32)cc1 ; c1ccc2c(c1)oc1ccc(-c3ccc4c5ccccc5n(-c5nc(-c6ccc7oc8ccccc8c7c6)nc(-c6cccc7c6oc6ccccc67)n5)c4c3)cc12 ; c1cc(-c2nc(-c3ccc4c(ccc5ccccc54)c3)nc(-c3ccc4oc5cccnc5c4c3)n2)cc(-n2c3ccccc3c3cc4ccccc4cc32)c1]

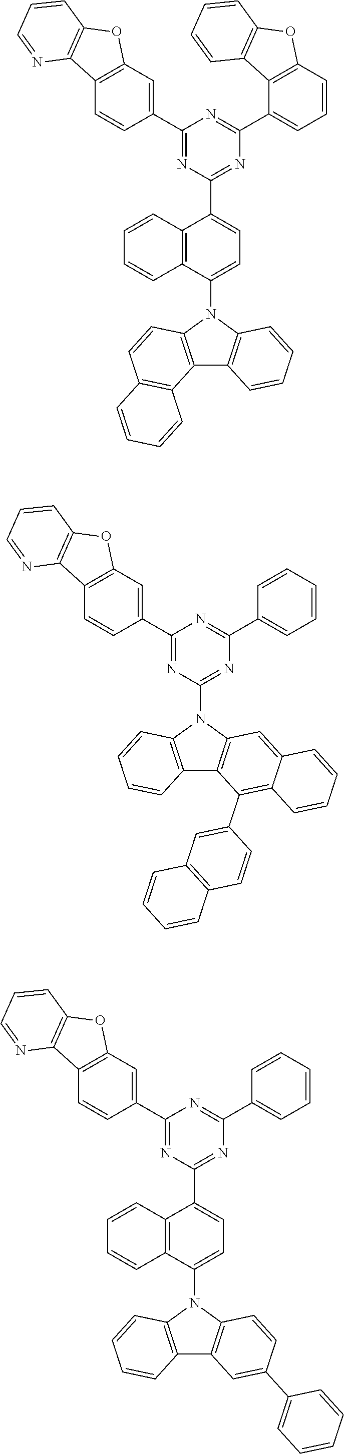 [SMILES: c1ccc2c(c1)ccc1c2c2ccccc2n1-c1ccc(-c2nc(-c3ccc4c(c3)oc3cccnc34)nc(-c3cccc4oc5ccccc5c34)n2)c2ccccc12 ; c1ccc(-c2nc(-c3ccc4c(c3)oc3cccnc34)nc(-n3c4ccccc4c4c(-c5ccc6ccccc6c5)c5ccccc5cc43)n2)cc1 ; c1ccc(-c2ccc3c(c2)c2ccccc2n3-c2ccc(-c3nc(-c4ccccc4)nc(-c4ccc5c(c4)oc4cccnc45)n3)c3ccccc23)cc1]

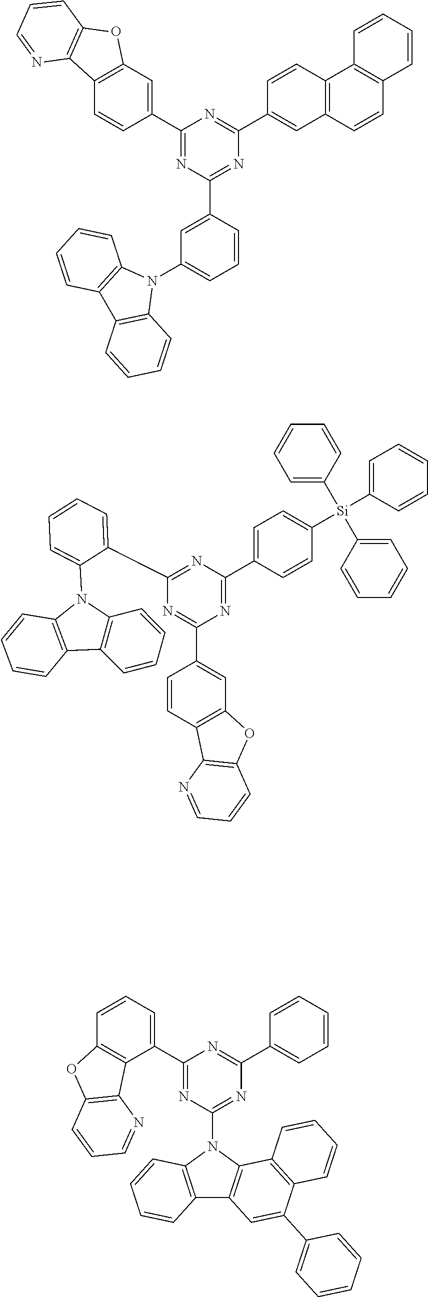 [SMILES: c1cc(-c2nc(-c3ccc4c(ccc5ccccc54)c3)nc(-c3ccc4c(c3)oc3cccnc34)n2)cc(-n2c3ccccc3c3ccccc32)c1 ; c1ccc([Si](c2ccccc2)(c2ccccc2)c2ccc(-c3nc(-c4ccc5c(c4)oc4cccnc45)nc(-c4ccccc4-n4c5ccccc5c5ccccc54)n3)cc2)cc1 ; c1ccc(-c2nc(-c3cccc4oc5cccnc5c34)nc(-n3c4ccccc4c4cc(-c5ccccc5)c5ccccc5c43)n2)cc1]

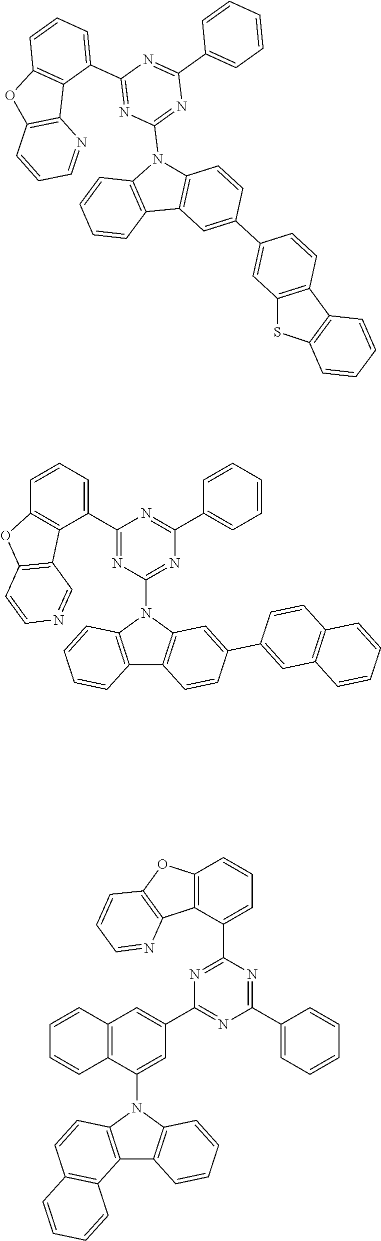 [SMILES: c1ccc(-c2nc(-c3cccc4oc5cccnc5c34)nc(-n3c4ccccc4c4cc(-c5ccc6c(c5)sc5ccccc56)ccc43)n2)cc1 ; c1ccc(-c2nc(-c3cccc4oc5ccncc5c34)nc(-n3c4ccccc4c4ccc(-c5ccc6ccccc6c5)cc43)n2)cc1 ; c1ccc(-c2nc(-c3cc(-n4c5ccccc5c5c6ccccc6ccc54)c4ccccc4c3)nc(-c3cccc4oc5cccnc5c34)n2)cc1]

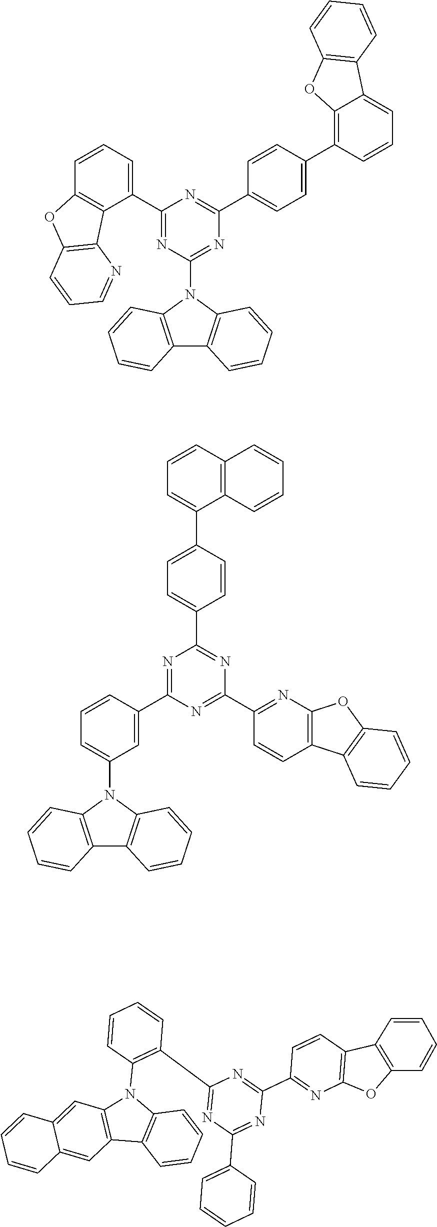 [SMILES: c1ccc2c(c1)oc1c(-c3ccc(-c4nc(-c5cccc6oc7cccnc7c56)nc(-n5c6ccccc6c6ccccc65)n4)cc3)cccc12 ; c1cc(-c2nc(-c3ccc(-c4cccc5ccccc45)cc3)nc(-c3ccc4c(n3)oc3ccccc34)n2)cc(-n2c3ccccc3c3ccccc32)c1 ; c1ccc(-c2nc(-c3ccc4c(n3)oc3ccccc34)nc(-c3ccccc3-n3c4ccccc4c4cc5ccccc5cc43)n2)cc1]

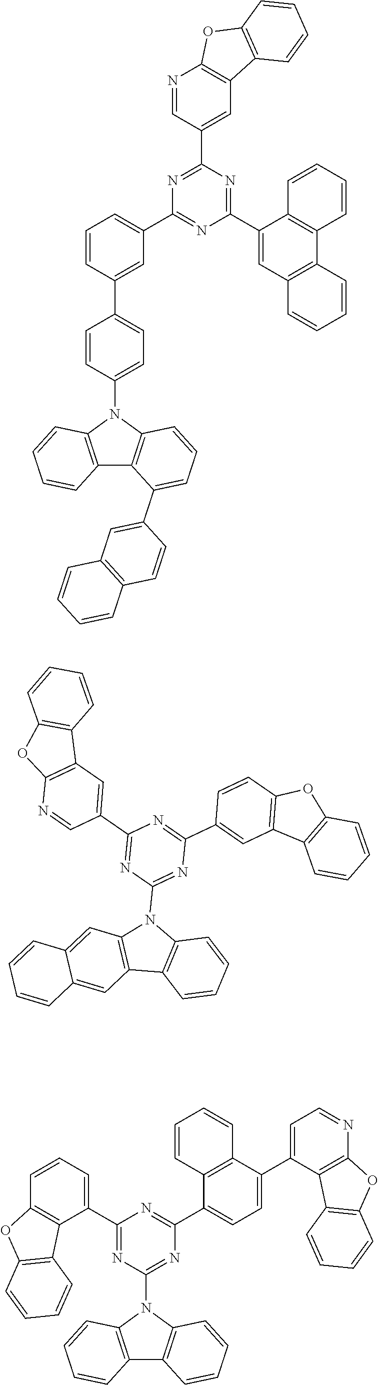 [SMILES: c1cc(-c2ccc(-n3c4ccccc4c4c(-c5ccc6ccccc6c5)cccc43)cc2)cc(-c2nc(-c3cnc4oc5ccccc5c4c3)nc(-c3cc4ccccc4c4ccccc34)n2)c1 ; c1ccc2cc3c(cc2c1)c1ccccc1n3-c1nc(-c2ccc3oc4ccccc4c3c2)nc(-c2cnc3oc4ccccc4c3c2)n1 ; c1ccc2c(c1)oc1cccc(-c3nc(-c4ccc(-c5ccnc6oc7ccccc7c56)c5ccccc45)nc(-n4c5ccccc5c5ccccc54)n3)c12]

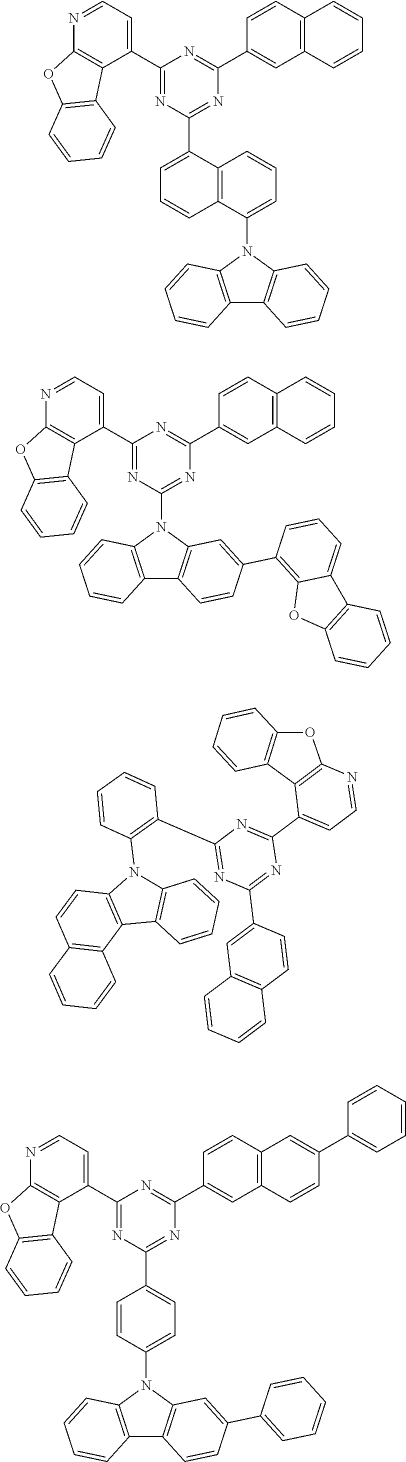 [SMILES: c1ccc2cc(-c3nc(-c4cccc5c(-n6c7ccccc7c7ccccc76)cccc45)nc(-c4ccnc5oc6ccccc6c45)n3)ccc2c1 ; c1ccc2cc(-c3nc(-c4ccnc5oc6ccccc6c45)nc(-n4c5ccccc5c5ccc(-c6cccc7c6oc6ccccc67)cc54)n3)ccc2c1 ; c1ccc(-n2c3ccccc3c3c4ccccc4ccc32)c(-c2nc(-c3ccc4ccccc4c3)nc(-c3ccnc4oc5ccccc5c34)n2)c1 ; c1ccc(-c2ccc3cc(-c4nc(-c5ccc(-n6c7ccccc7c7ccc(-c8ccccc8)cc76)cc5)nc(-c5ccnc6oc7ccccc7c56)n4)ccc3c2)cc1]

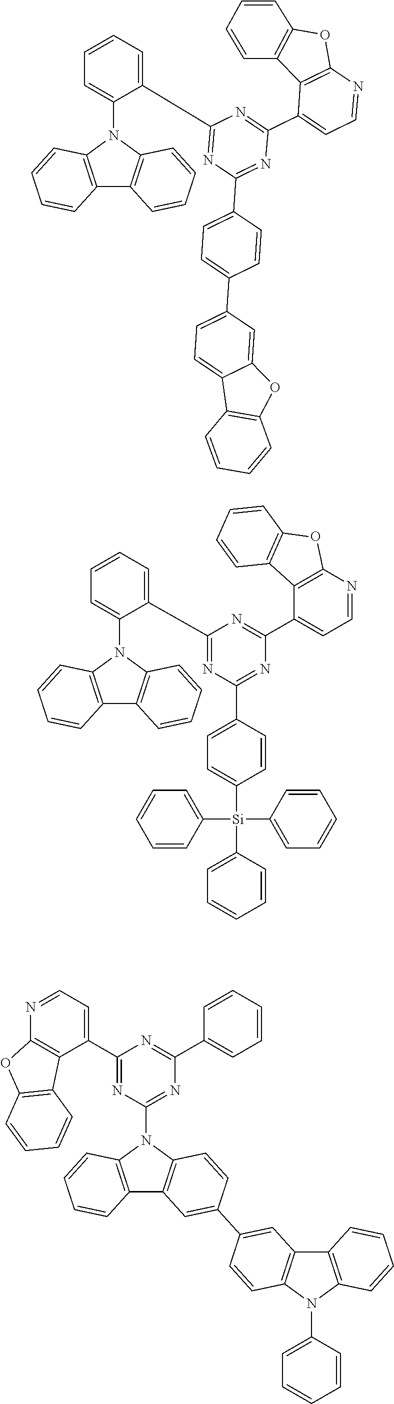

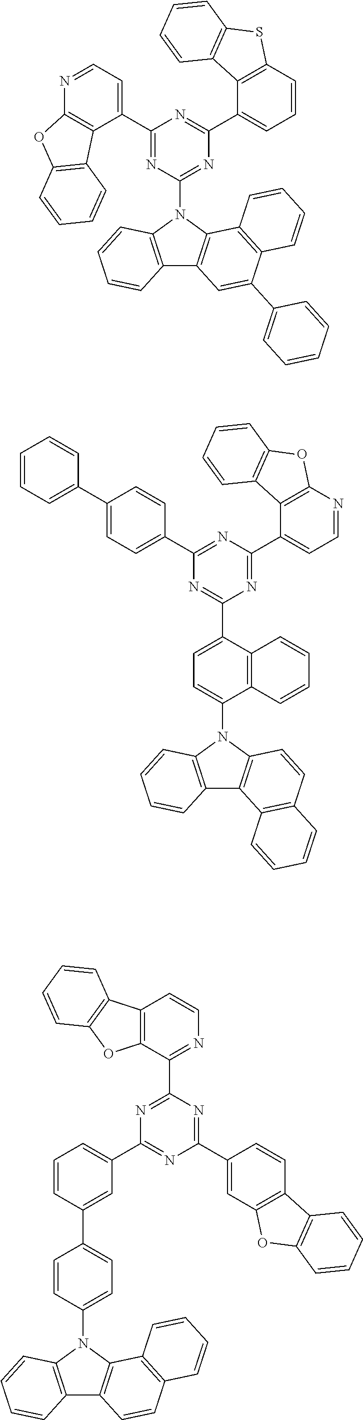 [SMILES: c1ccc(-c2cc3c4ccccc4n(-c4nc(-c5ccnc6oc7ccccc7c56)nc(-c5cccc6sc7ccccc7c56)n4)c3c3ccccc23)cc1 ; c1ccc(-c2ccc(-c3nc(-c4ccc(-n5c6ccccc6c6c7ccccc7ccc65)c5ccccc45)nc(-c4ccnc5oc6ccccc6c45)n3)cc2)cc1 ; c1cc(-c2ccc(-n3c4ccccc4c4ccc5ccccc5c43)cc2)cc(-c2nc(-c3ccc4c(c3)oc3ccccc34)nc(-c3nccc4c3oc3ccccc34)n2)c1]

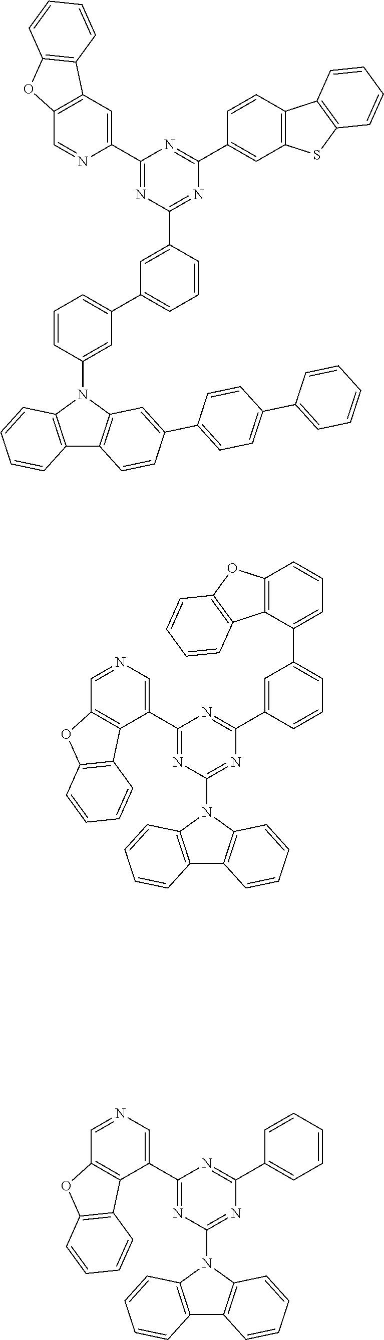 [SMILES: c1ccc(-c2ccc(-c3ccc4c5ccccc5n(-c5cccc(-c6cccc(-c7nc(-c8ccc9c(c8)sc8ccccc89)nc(-c8cc9c(cn8)oc8ccccc89)n7)c6)c5)c4c3)cc2)cc1 ; c1cc(-c2nc(-c3cncc4oc5ccccc5c34)nc(-n3c4ccccc4c4ccccc43)n2)cc(-c2cccc3oc4ccccc4c23)c1 ; c1ccc(-c2nc(-c3cncc4oc5ccccc5c34)nc(-n3c4ccccc4c4ccccc43)n2)cc1]

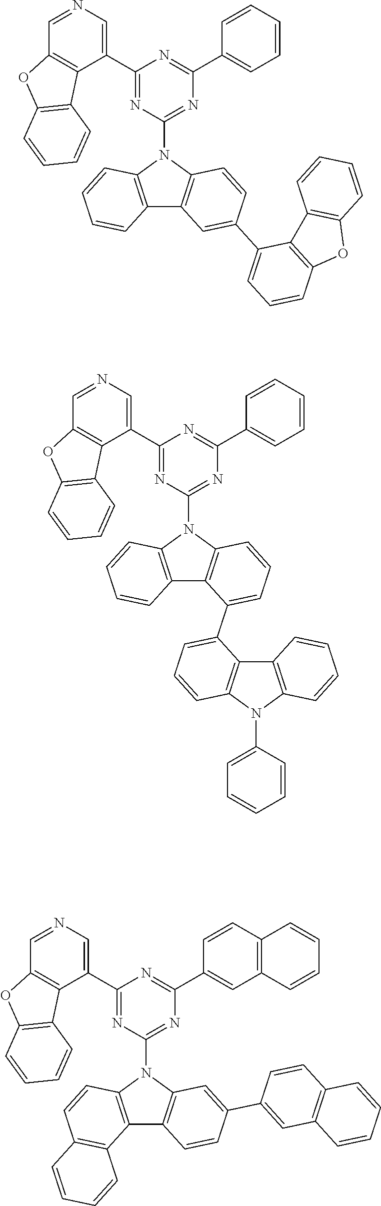 [SMILES: c1ccc(-c2nc(-c3cncc4oc5ccccc5c34)nc(-n3c4ccccc4c4cc(-c5cccc6oc7ccccc7c56)ccc43)n2)cc1 ; c1ccc(-c2nc(-c3cncc4oc5ccccc5c34)nc(-n3c4ccccc4c4c(-c5cccc6c5c5ccccc5n6-c5ccccc5)cccc43)n2)cc1 ; c1ccc2cc(-c3ccc4c5c6ccccc6ccc5n(-c5nc(-c6ccc7ccccc7c6)nc(-c6cncc7oc8ccccc8c67)n5)c4c3)ccc2c1]

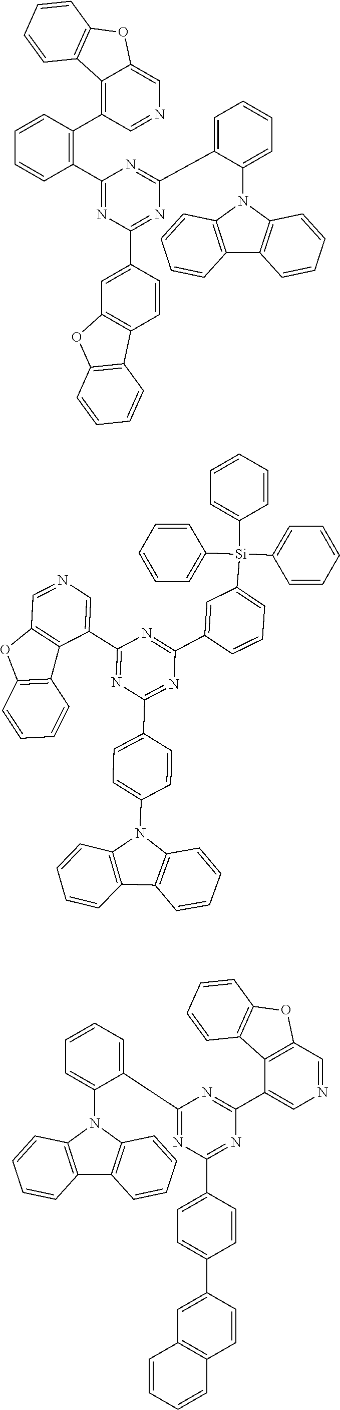 [SMILES: c1ccc(-c2cncc3oc4ccccc4c23)c(-c2nc(-c3ccc4c(c3)oc3ccccc34)nc(-c3ccccc3-n3c4ccccc4c4ccccc43)n2)c1 ; c1ccc([Si](c2ccccc2)(c2ccccc2)c2cccc(-c3nc(-c4ccc(-n5c6ccccc6c6ccccc65)cc4)nc(-c4cncc5oc6ccccc6c45)n3)c2)cc1 ; c1ccc(-n2c3ccccc3c3ccccc32)c(-c2nc(-c3ccc(-c4ccc5ccccc5c4)cc3)nc(-c3cncc4oc5ccccc5c34)n2)c1]

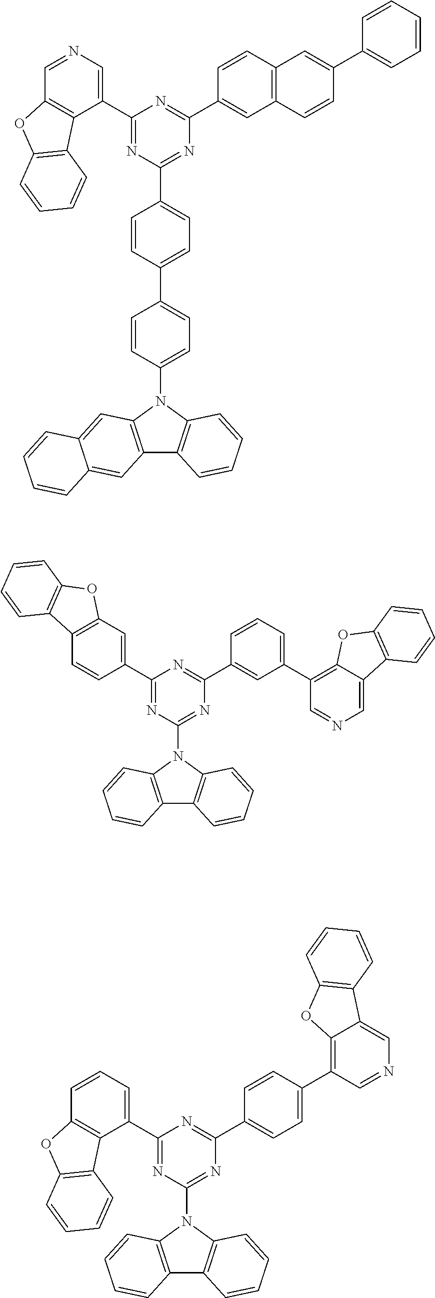 [SMILES: c1ccc(-c2ccc3cc(-c4nc(-c5ccc(-c6ccc(-n7c8ccccc8c8cc9ccccc9cc87)cc6)cc5)nc(-c5cncc6oc7ccccc7c56)n4)ccc3c2)cc1 ; c1cc(-c2nc(-c3ccc4c(c3)oc3ccccc34)nc(-n3c4ccccc4c4ccccc43)n2)cc(-c2cncc3c2oc2ccccc23)c1 ; c1ccc2c(c1)oc1c(-c3ccc(-c4nc(-c5cccc6oc7ccccc7c56)nc(-n5c6ccccc6c6ccccc65)n4)cc3)cncc12]

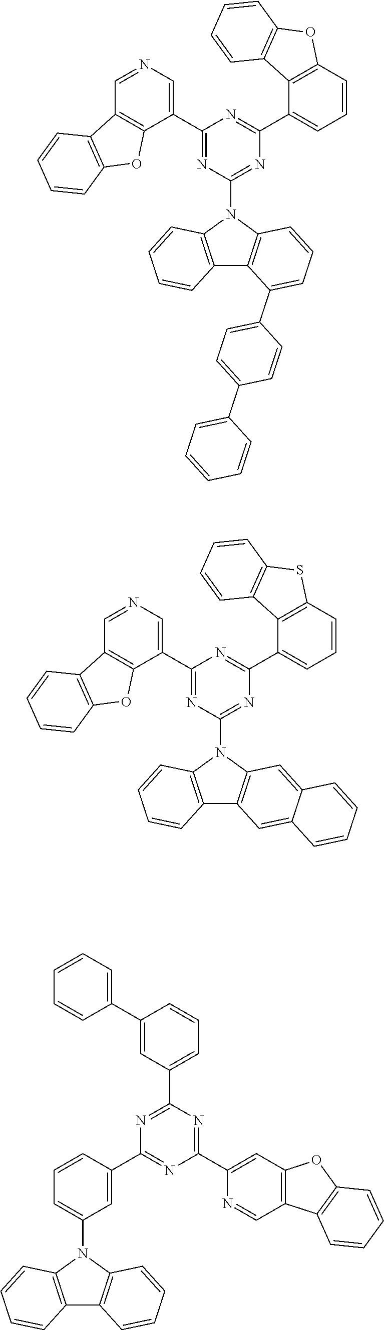 [SMILES: c1ccc(-c2ccc(-c3cccc4c3c3ccccc3n4-c3nc(-c4cncc5c4oc4ccccc45)nc(-c4cccc5oc6ccccc6c45)n3)cc2)cc1 ; c1ccc2cc3c(cc2c1)c1ccccc1n3-c1nc(-c2cncc3c2oc2ccccc23)nc(-c2cccc3sc4ccccc4c23)n1 ; c1ccc(-c2cccc(-c3nc(-c4cccc(-n5c6ccccc6c6ccccc65)c4)nc(-c4cc5oc6ccccc6c5cn4)n3)c2)cc1]

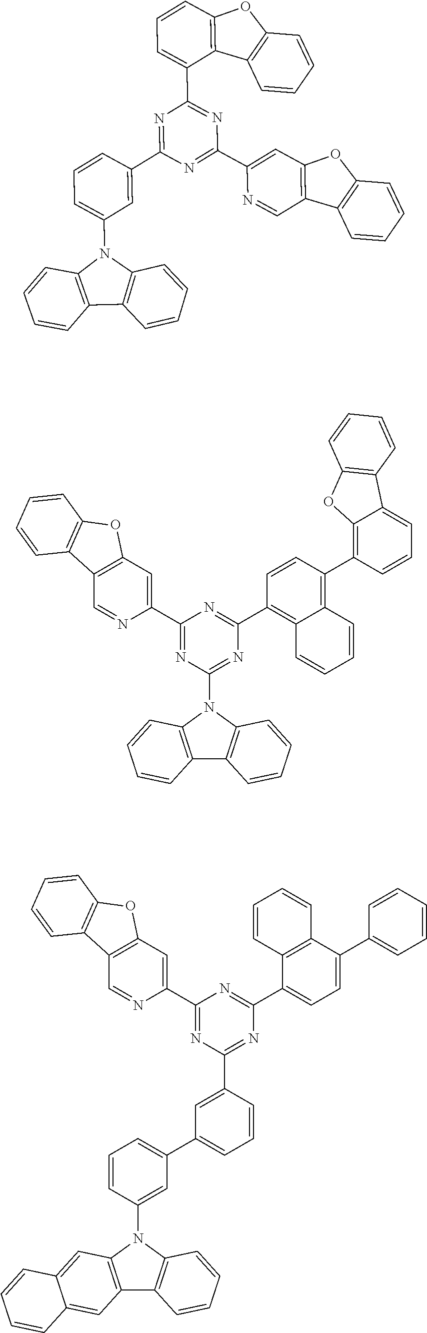 [SMILES: c1cc(-c2nc(-c3cc4oc5ccccc5c4cn3)nc(-c3cccc4oc5ccccc5c34)n2)cc(-n2c3ccccc3c3ccccc32)c1 ; c1ccc2c(c1)oc1cc(-c3nc(-c4ccc(-c5cccc6c5oc5ccccc56)c5ccccc45)nc(-n4c5ccccc5c5ccccc54)n3)ncc12 ; c1ccc(-c2ccc(-c3nc(-c4cccc(-c5cccc(-n6c7ccccc7c7cc8ccccc8cc76)c5)c4)nc(-c4cc5oc6ccccc6c5cn4)n3)c3ccccc23)cc1]

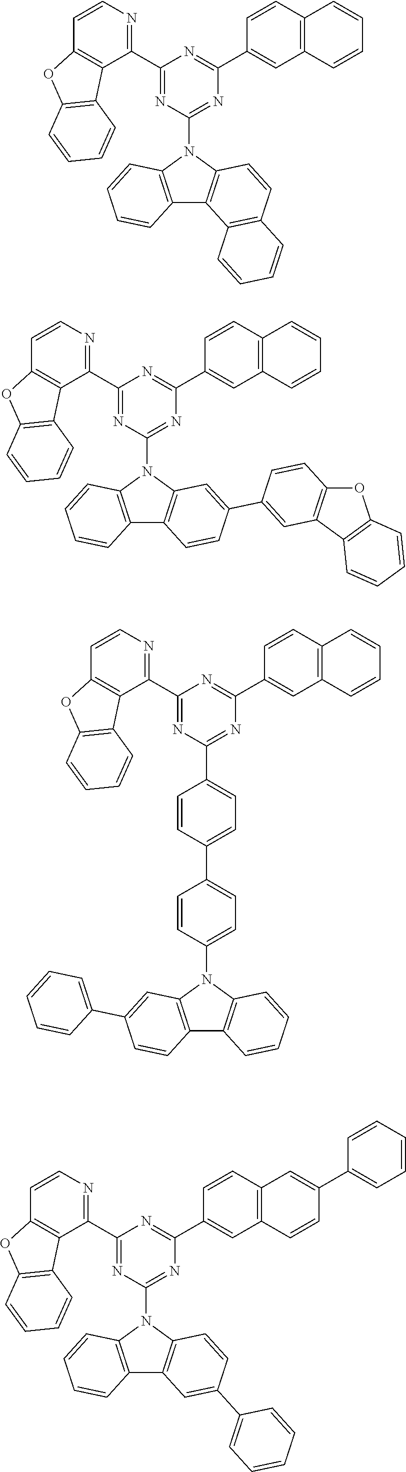

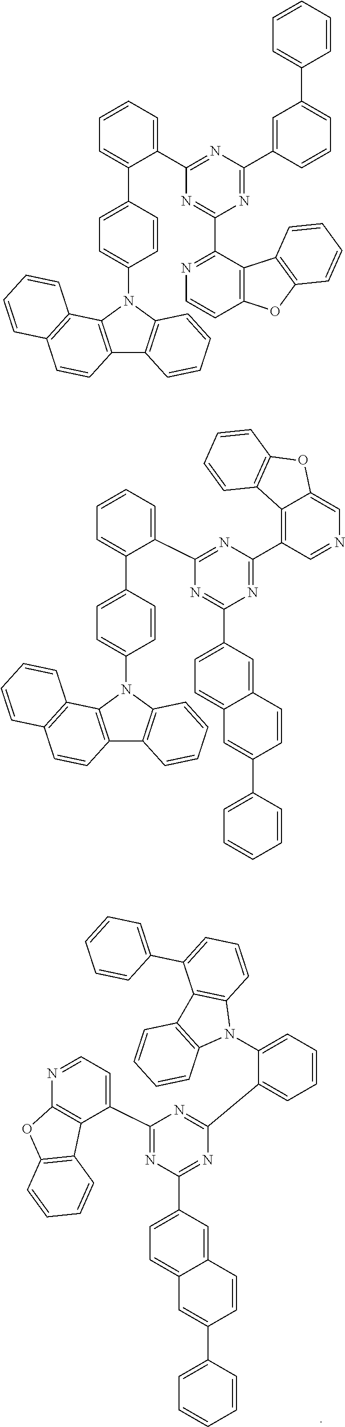 [SMILES: c1ccc(-c2cccc(-c3nc(-c4ccccc4-c4ccc(-n5c6ccccc6c6ccc7ccccc7c65)cc4)nc(-c4nccc5oc6ccccc6c45)n3)c2)cc1 ; c1ccc(-c2ccc3cc(-c4nc(-c5ccccc5-c5ccc(-n6c7ccccc7c7ccc8ccccc8c76)cc5)nc(-c5cncc6oc7ccccc7c56)n4)ccc3c2)cc1 ; c1ccc(-c2ccc3cc(-c4nc(-c5ccccc5-n5c6ccccc6c6c(-c7ccccc7)cccc65)nc(-c5ccnc6oc7ccccc7c56)n4)ccc3c2)cc1]

Further, the present disclosure provides a method for preparing the compound represented by Chemical Formula 1 as shown in the following Reaction Scheme 1.

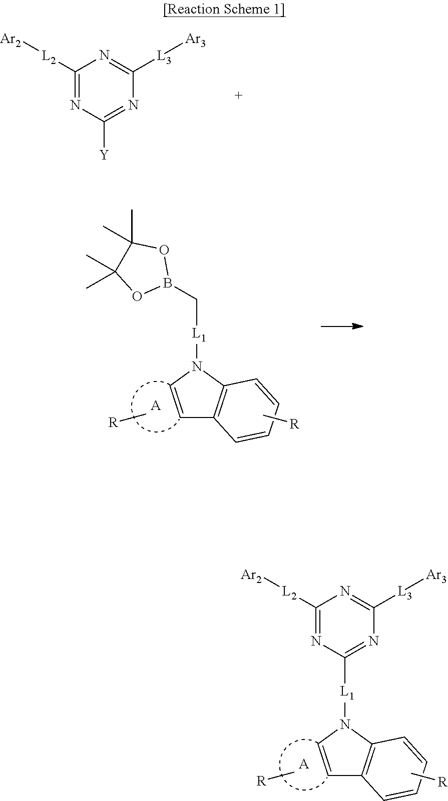

in Reaction Scheme 1, the remaining substituents except for Y are the same as defined above, and Y is halogen, more preferably bromo or chloro.

The above reaction is preferably carried out in the presence of a palladium catalyst and a base, and a reactive group for the reaction can be changed as known in the art. The above preparation method can be further embodied in Preparation Examples described hereinafter.

### Organic Light Emitting Device

Further, the present disclosure provides an organic light emitting device comprising a compound represented by Chemical Formula 1. In one example, the present disclosure provides an organic light emitting device comprising: a first electrode; a second electrode provided to face the first electrode; and an organic material layer including one or more layers provided between the first electrode and the second electrode, wherein one or more layers of the organic material layer include the compound represented by Chemical Formula 1.

The organic material layer of the organic light emitting device of the present disclosure may have a single-layer structure, or it may have a multilayered structure in which two or more organic material layers are stacked. For example, the organic light emitting device of the present disclosure may have a structure comprising a hole injection layer, a hole transport layer, a light emitting layer, an electron transport layer, an electron injection layer and the like as the organic material layer. However, the structure of the organic light emitting device is not limited thereto, and it may include a smaller number of organic layers.

Further, the organic material layer may include a light emitting layer, wherein the light emitting layer includes the compound represented by Chemical Formula 1. Particularly, the compound according to the present disclosure can be used as a dopant of the light emitting layer.

Further, the organic material layer may include an electron transport layer or an electron injection layer, wherein the electron transport layer or the electron injection layer includes the compound represented by Chemical Formula 1.

Further, the electron transport layer, the electron injection layer, or a layer for simultaneously performing electron transport and electron injection includes the compound represented by Chemical Formula 1.

Further, the organic material layer includes a light emitting layer or an electron transport layer, wherein the electron transport layer may include the compound represented by Chemical Formula 1.

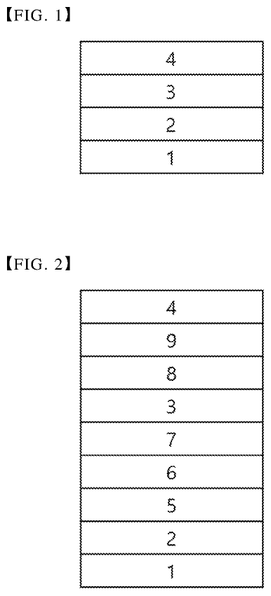

Further, the organic light emitting device according to the present disclosure may be a normal type organic light emitting device in which an anode, one or more organic material layers, and a cathode are sequentially stacked on a substrate. Further, the organic light emitting device according to the present disclosure may be an inverted type organic light emitting device in which a cathode, one or more organic material layers, and an anode are sequentially stacked on a substrate. For example, the structure of the organic light emitting device according to an embodiment of the present disclosure is illustrated in FIGS. 1 and 2.

FIG. 1 shows an example of an organic light emitting device comprising a substrate 1, an anode 2, a light emitting layer 3, and a cathode 4. In such a structure, the compound represented by Chemical Formula 1 may be included in the light emitting layer.

FIG. 2 shows an example of an organic light emitting device comprising a substrate 1, an anode 2, a hole injection layer 5, a hole transport layer 6, a second hole transport layer 7, a light emitting layer 3, a hole blocking layer 8, an electron injection and transport layer 9, and a cathode 4. In such a structure, the compound represented by Chemical Formula 1 may be included in the light emitting layer.

The organic light emitting device according to the present disclosure may be manufactured by materials and methods known in the art, except that at least one of the organic material layers includes the compound represented by Chemical Formula 1. Further, when the organic light emitting device includes a plurality of organic material layers, the organic material layers can be formed of the same material or different materials.

For example, the organic light emitting device according to the present disclosure can be manufactured by sequentially stacking a first electrode, an organic material layer and a second electrode on a substrate. In this case, the organic light emitting device may be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound represented by Chemical Formula 1 can be formed into an organic layer by a solution coating method as well as a vacuum deposition method at the time of manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device may be manufactured by sequentially depositing a cathode material, an organic material layer and an anode material on a substrate (International Publication WO2003/012890). However, the manufacturing method is not limited thereto.

As an example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof: metal oxides such as zinc oxides, indium oxides, indium tin oxides (ITO), and indium zinc oxides (IZO); a combination of metals and oxides, such as ZnO:Al or SnO₂:Sb: conductive compounds such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof: a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which may receive holes from the anode or the hole injection layer and transfer the holes to the light emitting layer. Specific examples thereof include an arylamine-based organic material, a conductive polymer, a block copolymer in which a conjugate portion and a non-conjugate portion are present together, and the like, but are not limited thereto.

The light emitting material is preferably a material which may receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include an 8-hydroxy-quinoline aluminum complex (Alq₃); a carbazole-based compound; a dimerized styryl compound; BAlq; a 10-hydroxybenzoquinoline-metal compound; a benzoxazole, benzthiazole and benzimidazole-based compound; a poly(p-phenylenevinylene)(PPV)-based polymer; a spiro compound; polyfluorene, lubrene, and the like, but are not limited thereto.

The light emitting layer may include a host material and a dopant material. The host material may be a fused aromatic ring derivative, a heterocycle-containing compound or the like. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound of an arylamine, which is unsubstituted or substituted with one or two or more substituent groups selected from the group consisting of an aryl group, a silyl group, an alkyl group, a cycloalkyl group, and an arylamino group, and which is substituted with at least one arylvinyl group. Specific examples thereof include styrylamine, styryldiamine, styryltriamine, styryltetramine, and the like, but are not limited thereto. Further, the metal complex includes an iridium complex, a platinum complex, and the like, but is not limited thereto.

The hole blocking layer refers to a layer which is formed on the light emitting layer, preferably provided in contact with the light emitting layer, and serves to adjust the electron mobility, prevent excessive movement of holes, and increase the probability of hole-electron coupling, thereby improving the efficiency of the organic light emitting device. The hole blocking layer includes a hole blocking material, and examples of such hole blocking material may include a compound having an electron withdrawing group introduced therein, such as azine derivatives including triazine; triazole derivatives; oxadiazole derivatives; phenanthroline derivatives; phosphine oxide derivatives, but is not limited thereto.

The electron injection and transport layer is a layer for simultaneously performing the roles of an electron transport layer and an electron injection layer that inject electrons from an electrode and transport the received electrons up to the light emitting layer, and is formed on the light emitting layer or the hole blocking layer. The electron injection and transport material is suitably a material which can receive electrons well from a cathode and transfer the electrons to a light emitting layer, and has a large mobility for electrons. Specific examples of the electron injection and transport material include: an Al complex of 8-hydroxyquinoline; a complex including Alq₃; an organic radical compound; a hydroxyflavone-metal complex, a triazine derivative, and the like, but are not limited thereto. Alternatively, it may be used together with fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

The electron injection and transport layer may also be formed as a separate layer such as an electron injection layer and an electron transport layer. In such a case, the electron transport layer is formed on the light emitting layer or the hole blocking layer, and the above-mentioned electron injection and transport material may be used as the electron transport material included in the electron transport layer. In addition, the electron injection layer is formed on the electron transport layer, and examples of the electron injection material included in the electron injection layer include LiF, NaCl, CsF, LizO, BaO, fluorenone, anthraquinodimethane, diphenoquinone, thiopyran dioxide, oxazole, oxadiazole, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like.

Examples of the metal complex compound include 8-hydroxyquinolinato lithium, bis(8-hydroxyquinolinato)zinc, bis(8-hydroxyquinolinato)copper, bis(8-hydroxyquinolinato) manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

The organic light emitting device according to the present disclosure may be a bottom emission type device, a top emission type device, or a double side emission type device, and in particular, it may be a bottom emission type light emitting device that requires relatively high luminous efficiency.

In addition, the compound represented by Chemical Formula 1 may be included in an organic solar cell or an organic transistor in addition to an organic light emitting device.

The preparation of the compound represented by Chemical Formula 1 and the organic light emitting device including the same will be described in detail in the following examples. However, these examples are presented for illustrative purposes only, and are not intended to limit the scope of the present disclosure.

### SYNTHESIS EXAMPLE

### Synthesis Example 1

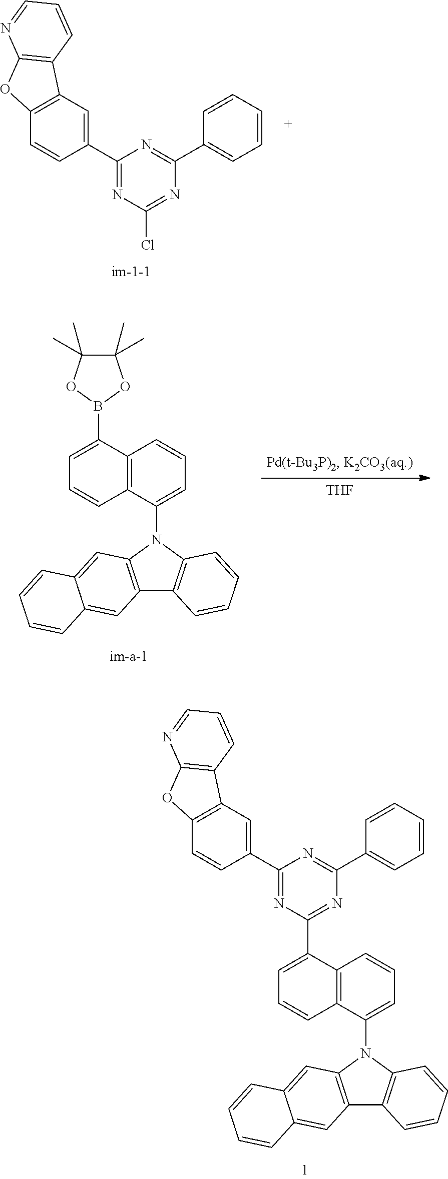 [SMILES: Clc1nc(-c2ccccc2)nc(-c2ccc3oc4ncccc4c3c2)n1 ; CC1(C)OB(c2cccc3c(-n4c5ccccc5c5cc6ccccc6cc54)cccc23)OC1(C)C ; "Pd(t-Bu3P)2, K2CO3(aq.)" ; "THF" ; c1ccc(-c2nc(-c3ccc4oc5ncccc5c4c3)nc(-c3cccc4c(-n5c6ccccc6c6cc7ccccc7cc65)cccc34)n2)cc1]

im-1-1 (15 g, 41.8 mmol) and im-a-1 (20.6 g, 43.9 mmol) were added to 300 ml of THF, and the mixture was stirred and refluxed. Then, potassium carbonate (17.3 g, 125.4 mmol) was dissolved in 100 ml of water and added thereto, and the mixture was sufficiently stirred and bis(tri-tert-butylphosphine)palladium(0) (0.2 g, 0.4 mmol) was added. After the reaction for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 19.7 g of Compound 1. (Yield: 71%, MS: [M+H]+=666)

### Synthesis Example 2

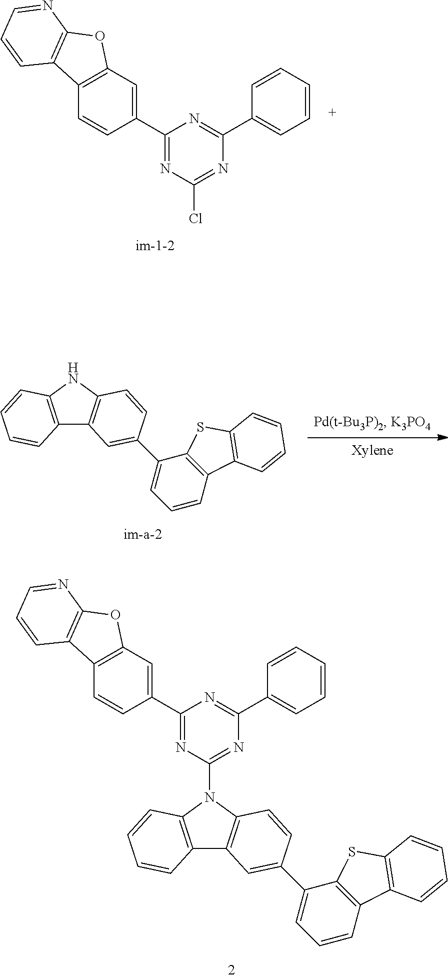

im-1-2 (15 g, 41.8 mmol), im-a-2 (15.3 g, 43.9 mmol) and potassium phosphate (26.6 g, 125.4 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 4 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 18.2 g of Compound 2. (Yield: 65%, MS: [M+H]+=672)

### Synthesis Example 3

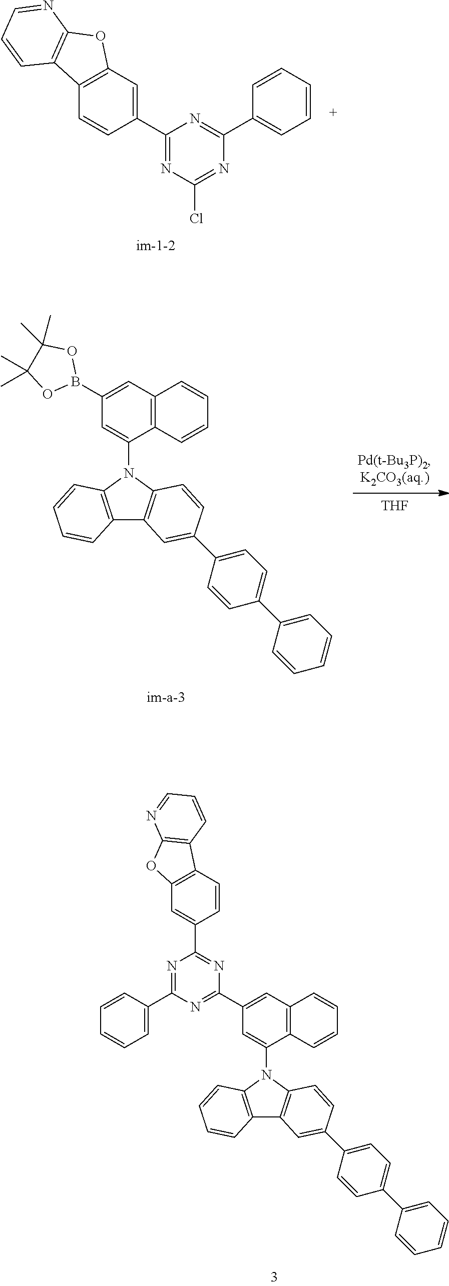 [SMILES: Clc1nc(-c2ccccc2)nc(-c2ccc3c(c2)oc2ncccc23)n1 ; CC1(C)OB(c2cc(-n3c4ccccc4c4cc(-c5ccc(-c6ccccc6)cc5)ccc43)c3ccccc3c2)OC1(C)C ; "Pd(t-Bu3P)2,K2CO3(aq.)" ; "THF" ; c1ccc(-c2ccc(-c3ccc4c(c3)c3ccccc3n4-c3cc(-c4nc(-c5ccccc5)nc(-c5ccc6c(c5)oc5ncccc56)n4)cc4ccccc34)cc2)cc1]

im-1-2 (15 g, 41.8 mmol) and im-a-3 (25.1 g, 43.9 mmol) were added to 300 ml of THE, and the mixture was stirred and refluxed. Then, potassium carbonate (17.3 g, 125.4 mmol) was dissolved in 100 ml of water and added thereto, and the mixture was sufficiently stirred and bis(tri-tert-butylphosphine)palladium(0) (0.2 g, 0.4 mmol) was added. After the reaction for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 23.4 g of Compound 3. (Yield: 73%, MS: [M+H]+=768)

### Synthesis Example 4

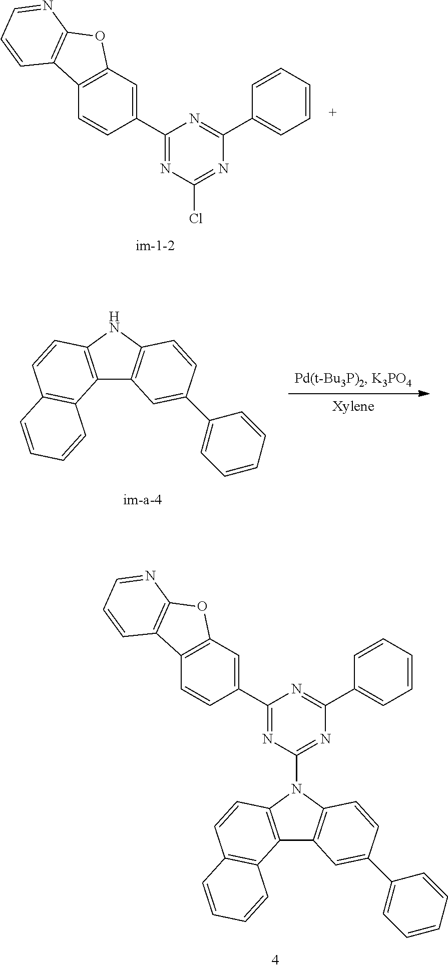

im-1-2 (15 g, 41.8 mmol), im-a-4 (12.9 g, 43.9 mmol) and potassium phosphate (26.6 g, 125.4 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 17.2 g of Compound 4. (Yield: 67%, MS: [M+H]+=616)

### Synthesis Example 5

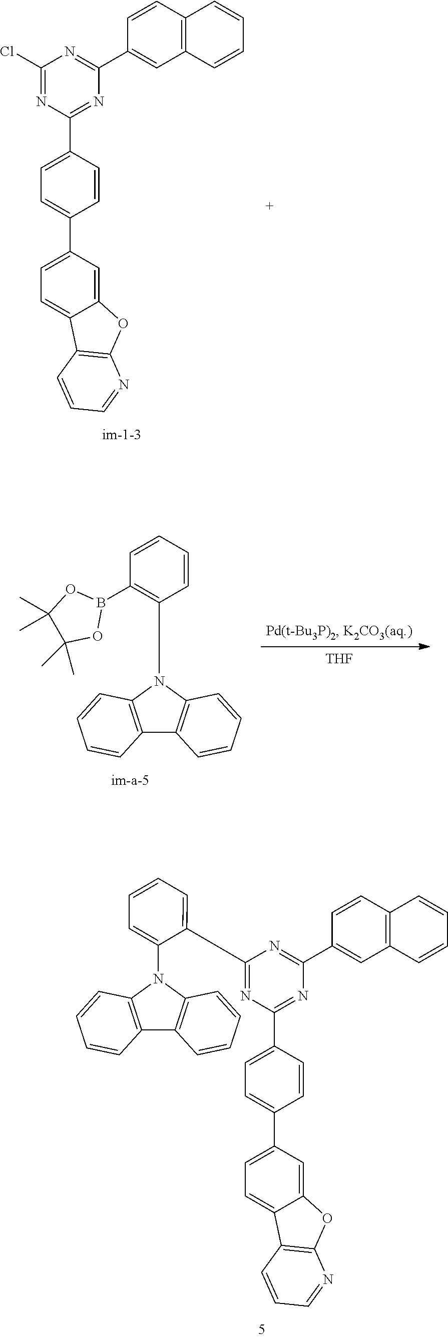 [SMILES: Clc1nc(-c2ccc(-c3ccc4c(c3)oc3ncccc34)cc2)nc(-c2ccc3ccccc3c2)n1 ; CC1(C)OB(c2ccccc2-n2c3ccccc3c3ccccc32)OC1(C)C ; "Pd(t-Bu3P)2, K2CO3(aq.)" ; "THF" ; c1ccc(-n2c3ccccc3c3ccccc32)c(-c2nc(-c3ccc(-c4ccc5c(c4)oc4ncccc45)cc3)nc(-c3ccc4ccccc4c3)n2)c1]

im-1-3 (15 g, 30.9 mmol) and im-a-5 (12 g, 32.5 mmol) were added to 300 ml of THF, and the mixture was stirred and refluxed. Then, potassium carbonate (12.8 g, 92.8 mmol) was dissolved in 100 ml of water and added thereto, and the mixture was sufficiently stirred and bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.7 g of Compound 5. (Yield: 64%, MS: [M+H]+=692)

### Synthesis Example 6

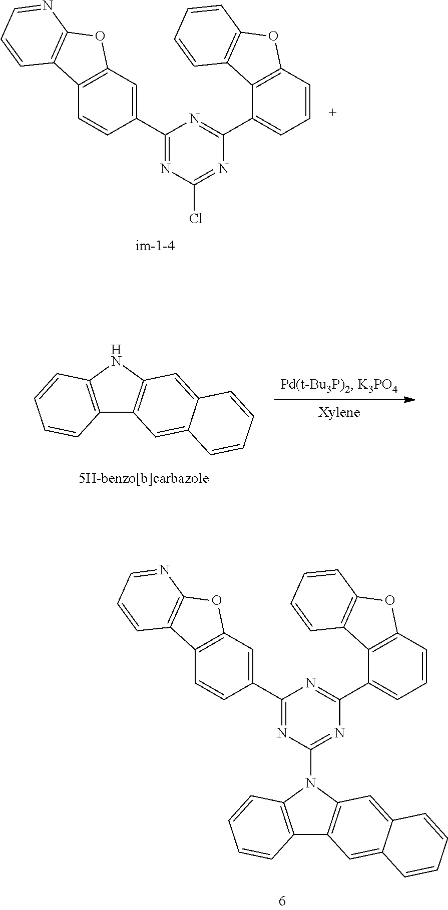

im-1-4 (15 g, 33.4 mmol), 5H-benzo[b]carbazole (7.6 g, 35.1 mmol), and potassium phosphate (21.3 g, 100.3 mmol) were added to 300 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 14.1 g of Compound 6. (Yield: 67%, MS: [M+H]+=630)

### Synthesis Example 7

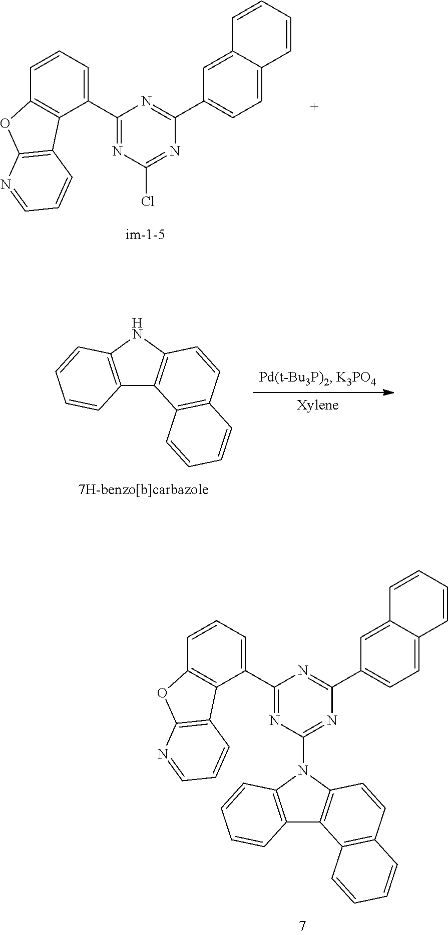

im-1-5 (15 g, 36.7 mmol), 7H-benzo[c]carbazole (8.4 g, 38.5 mmol) and potassium phosphate (23.4 g, 110.1 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 14.7 g of Compound 7. (Yield: 68%, MS: [M+H]+=590)

### Synthesis Example 8

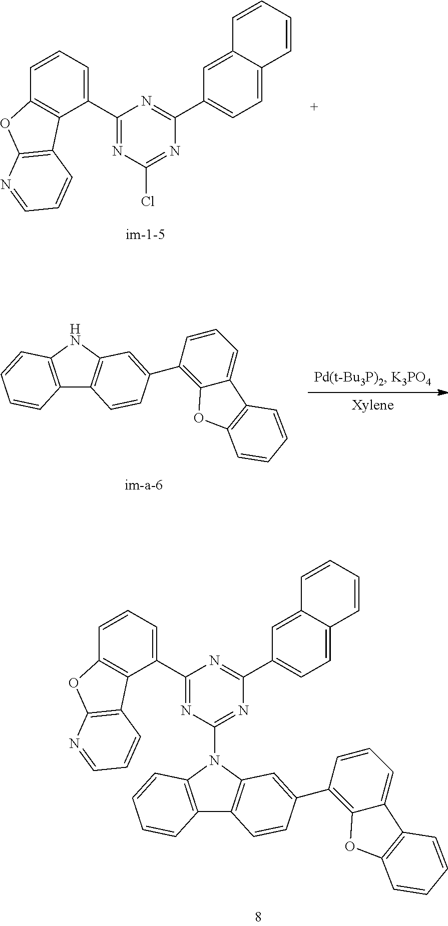

im-1-5 (15 g, 36.7 mmol), im-a-6 (12.8 g, 38.5 mmol) and potassium phosphate (23.4 g, 110.1 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 19.1 g of Compound 8. (Yield: 74%, MS: [M+H]+=706)

### Synthesis Example 9

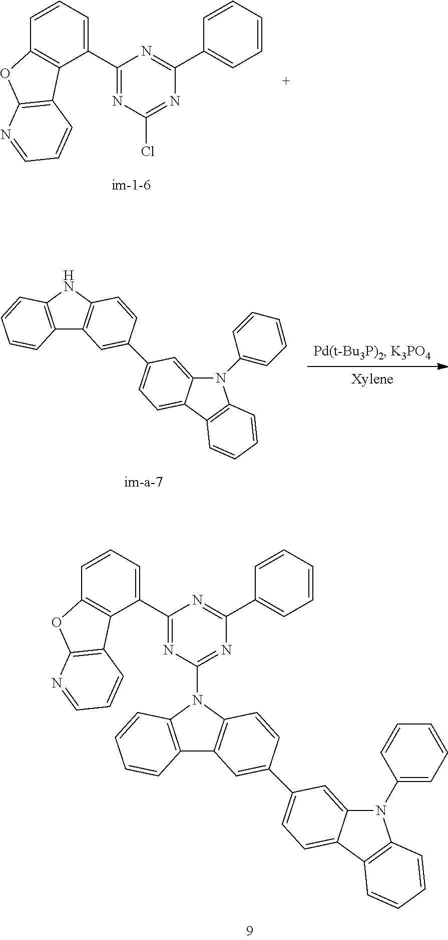

im-1-6 (15 g, 41.8 mmol), im-a-7 (17.9 g, 43.9 mmol) and potassium phosphate (26.6 g, 125.4 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 18.9 g of Compound 9. (Yield: 62%, MS: [M+H]+=731)

### Synthesis Example 10

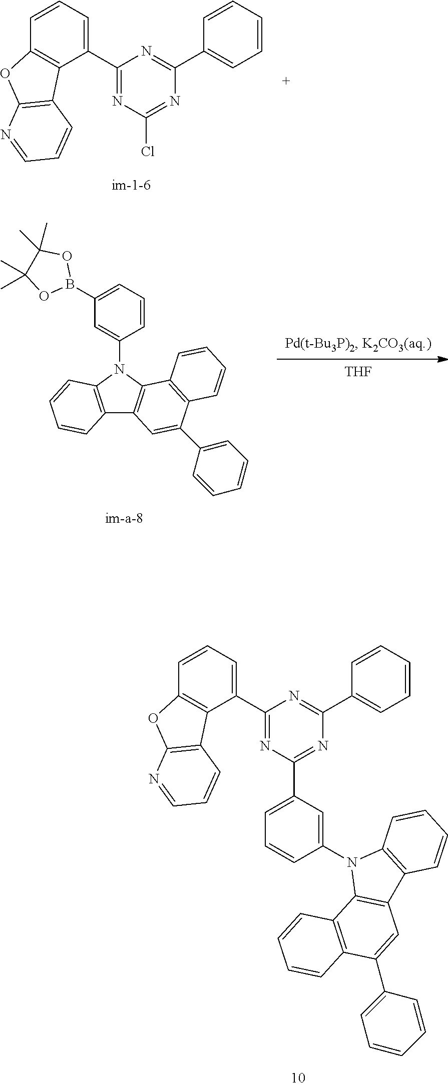 [SMILES: Clc1nc(-c2ccccc2)nc(-c2cccc3oc4ncccc4c23)n1 ; CC1(C)OB(c2cccc(-n3c4ccccc4c4cc(-c5ccccc5)c5ccccc5c43)c2)OC1(C)C ; "Pd(t-Bu3P)2, K2CO3(aq.)" ; "THF" ; c1ccc(-c2nc(-c3cccc(-n4c5ccccc5c5cc(-c6ccccc6)c6ccccc6c54)c3)nc(-c3cccc4oc5ncccc5c34)n2)cc1]

im-1-6 (15 g, 41.8 mmol) and im-a-8 (21.7 g, 43.9 mmol) were added to 300 ml of THF, and the mixture was stirred and refluxed. Then, potassium carbonate (17.3 g, 125.4 mmol) was dissolved in 100 ml of water and added thereto, and the mixture was sufficiently stirred and bis(tri-tert-butylphosphine)palladium(0) (0.2 g, 0.4 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 17.6 g of Compound 10. (Yield: 61%, MS: [M+H]+=692)

### Synthesis Example 11

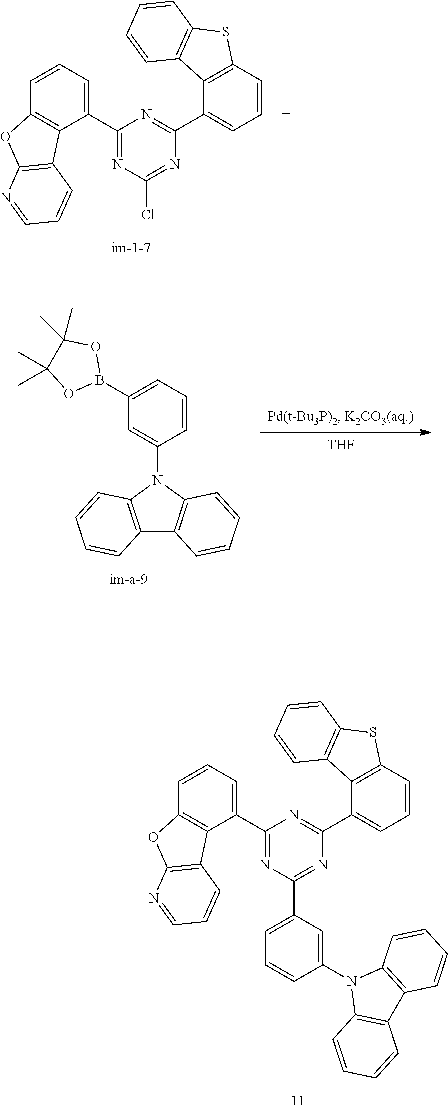 [SMILES: Clc1nc(-c2cccc3oc4ncccc4c23)nc(-c2cccc3sc4ccccc4c23)n1 ; CC1(C)OB(c2cccc(-n3c4ccccc4c4ccccc43)c2)OC1(C)C ; "Pd(t-Bu3P)2, K2CO3(aq.)" ; "THF" ; c1cc(-c2nc(-c3cccc4oc5ncccc5c34)nc(-c3cccc4sc5ccccc5c34)n2)cc(-n2c3ccccc3c3ccccc32)c1]

im-1-7 (15 g, 32.3 mmol) and im-a-9 (12.5 g, 33.9 mmol) were added to 300 ml of THF, and the mixture was stirred and refluxed. Then, potassium carbonate (13.4 g, 96.8 mmol) was dissolved in 100 ml of water and added thereto, and the mixture was sufficiently stirred and bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.6 g of Compound 11. (Yield: 72%, MS: [M+H]+=672)

### Synthesis Example 12

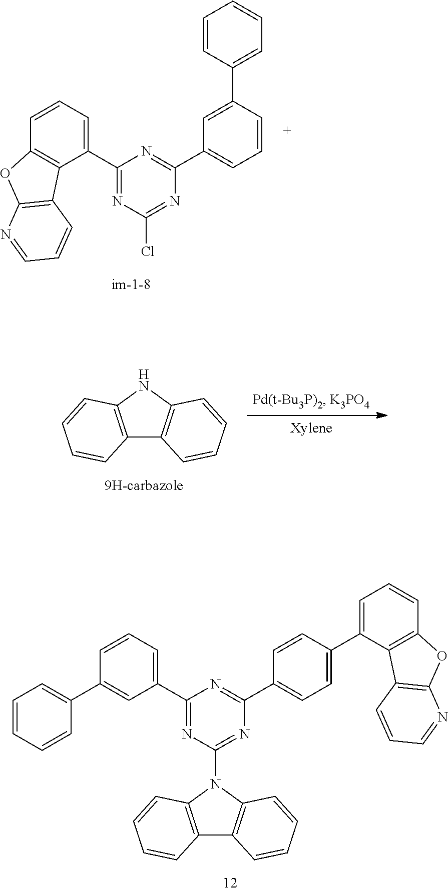

im-1-8 (15 g, 34.5 mmol), 9H-carbazole (6.1 g, 36.2 mmol) and potassium phosphate (22 g, 103.5 mmol) were added to 300 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 4 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 15.9 g of Compound 12. (Yield: 72%, MS: [M+H]+=642)

### Synthesis Example 13

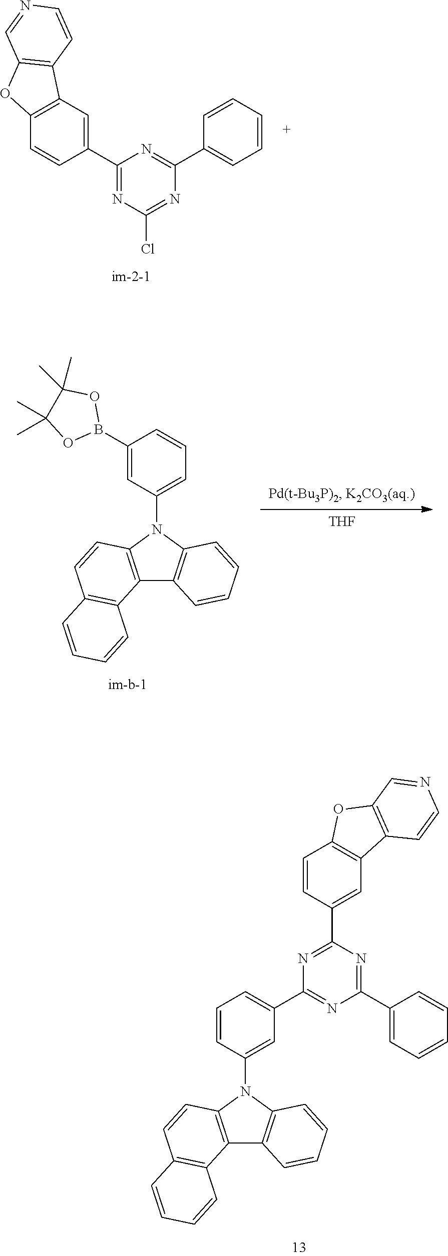 [SMILES: Clc1nc(-c2ccccc2)nc(-c2ccc3oc4cnccc4c3c2)n1 ; CC1(C)OB(c2cccc(-n3c4ccccc4c4c5ccccc5ccc43)c2)OC1(C)C ; "Pd(t-Bu3P)2, K2CO3(aq.)" ; "THF" ; c1ccc(-c2nc(-c3cccc(-n4c5ccccc5c5c6ccccc6ccc54)c3)nc(-c3ccc4oc5cnccc5c4c3)n2)cc1]

im-2-1 (15 g, 41.8 mmol) and im-b-1 (18.4 g, 43.9 mmol) were added to 300 ml of THE, and the mixture was stirred and refluxed. Then, potassium carbonate (17.3 g, 125.4 mmol) was dissolved in 100 ml of water and added thereto, and the mixture was sufficiently stirred and bis(tri-tert-butylphosphine)palladium(0) (0.2 g, 0.4 mmol) was added. After the reaction for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.2 g of Compound 13. (Yield: 63%, MS: [M+H]+=616)

### Synthesis Example 14

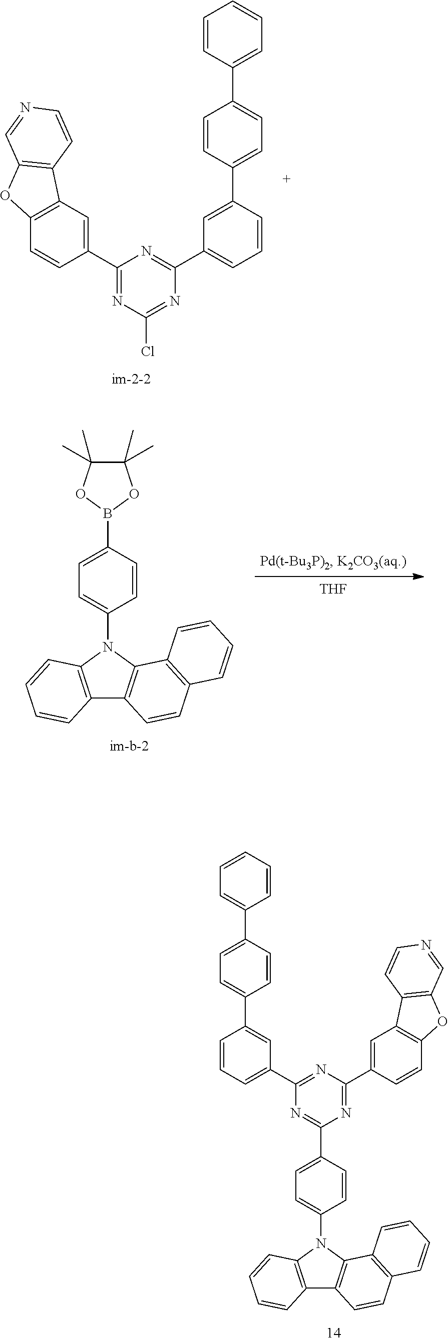 [SMILES: Clc1nc(-c2cccc(-c3ccc(-c4ccccc4)cc3)c2)nc(-c2ccc3oc4cnccc4c3c2)n1 ; CC1(C)OB(c2ccc(-n3c4ccccc4c4ccc5ccccc5c43)cc2)OC1(C)C ; "Pd(t-Bu3P)2, K2CO3(aq.)" ; "THF" ; c1ccc(-c2ccc(-c3cccc(-c4nc(-c5ccc(-n6c7ccccc7c7ccc8ccccc8c76)cc5)nc(-c5ccc6oc7cnccc7c6c5)n4)c3)cc2)cc1]

im-2-2 (15 g, 29.4 mmol) and im-b-2 (12.9 g, 30.8 mmol) were added to 300 ml of THF, and the mixture was stirred and refluxed. Then, potassium carbonate (12.2 g, 88.1 mmol) was dissolved in 100 ml of water and added thereto, and the mixture was sufficiently stirred and bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.9 g of Compound 14. (Yield: 75%, MS: [M+H]+=768)

### Synthesis Example 15

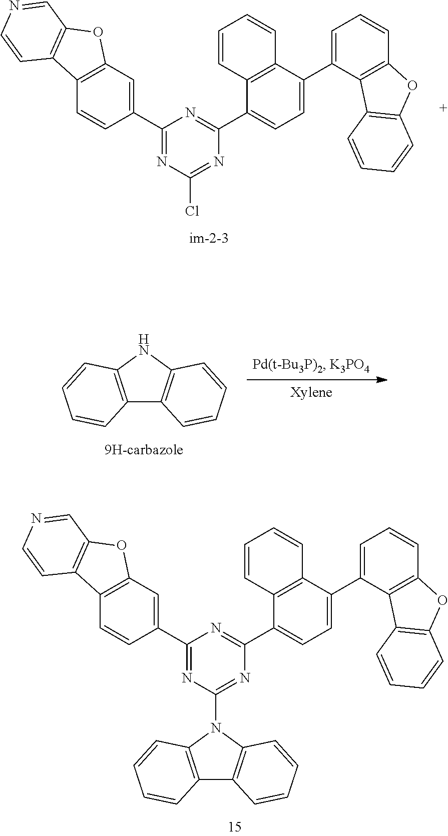

im-2-3 (15 g, 26.1 mmol), 9H-carbazole (4.6 g, 27.4 mmol) and potassium phosphate (16.6 g, 78.3 mmol) were added to 300 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.2 g of Compound 15. (Yield: 61%, MS: [M+H]+=706)

### Synthesis Example 16

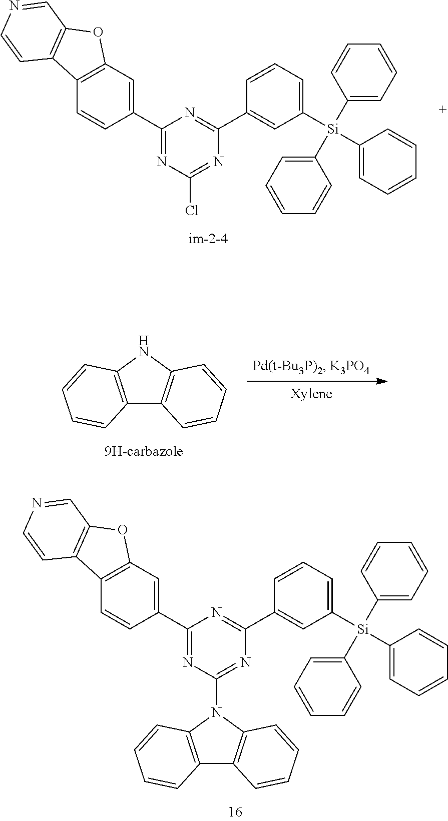

im-2-4 (15 g, 24.3 mmol), 9H-carbazole (4.3 g, 25.5 mmol) and potassium phosphate (15.5 g, 72.9 mmol) were added to 300 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.4 g of Compound 16. (Yield: 63%, MS: [M+H]+=748)

### Synthesis Example 17

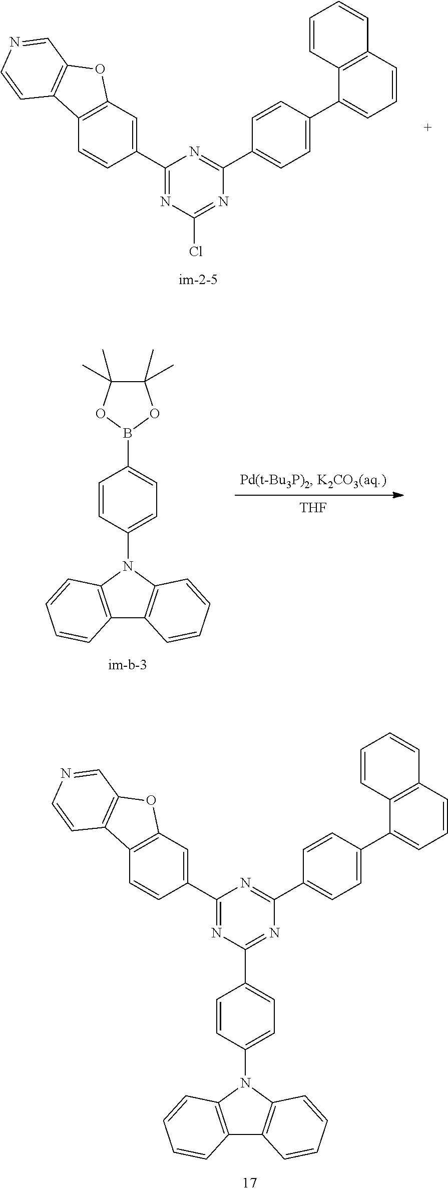 [SMILES: Clc1nc(-c2ccc(-c3cccc4ccccc34)cc2)nc(-c2ccc3c(c2)oc2cnccc23)n1 ; CC1(C)OB(c2ccc(-n3c4ccccc4c4ccccc43)cc2)OC1(C)C ; "Pd(t-Bu3P)2, K2CO3(aq.)" ; "THF" ; c1ccc2c(-c3ccc(-c4nc(-c5ccc(-n6c7ccccc7c7ccccc76)cc5)nc(-c5ccc6c(c5)oc5cnccc56)n4)cc3)cccc2c1]

im-2-5 (15 g, 30.9 mmol) and im-b-3 (12 g, 32.5 mmol) were added to 300 ml of THF, and the mixture was stirred and refluxed. Then, potassium carbonate (12.8 g, 92.8 mmol) was dissolved in 100 ml of water and added thereto, and the mixture was sufficiently stirred and bis(tri-tert-butylphosphine)palladium(0) (0.2 g, 0.3 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.6 g of Compound 17. (Yield: 73%, MS: [M+H]+=692)

### Synthesis Example 18

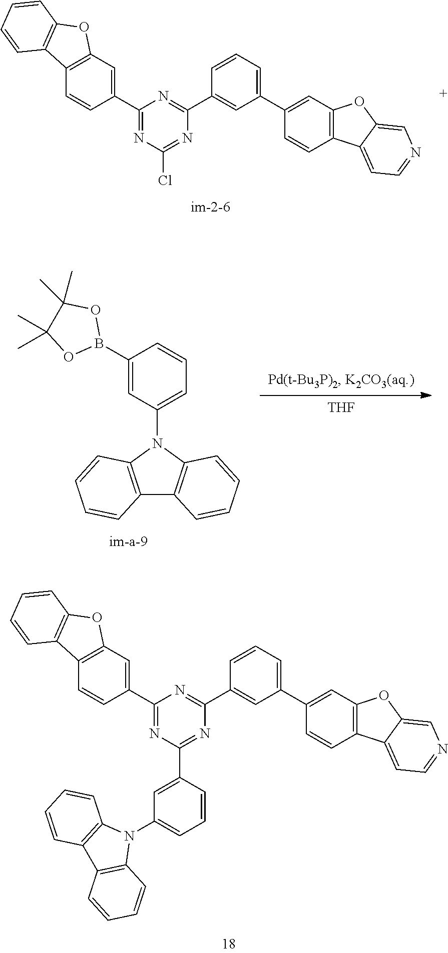 [SMILES: Clc1nc(-c2cccc(-c3ccc4c(c3)oc3cnccc34)c2)nc(-c2ccc3c(c2)oc2ccccc23)n1 ; CC1(C)OB(c2cccc(-n3c4ccccc4c4ccccc43)c2)OC1(C)C ; "Pd(t-Bu3P)2, K2CO3(aq.)" ; "THF" ; c1cc(-c2ccc3c(c2)oc2cnccc23)cc(-c2nc(-c3cccc(-n4c5ccccc5c5ccccc54)c3)nc(-c3ccc4c(c3)oc3ccccc34)n2)c1]

im-2-6 (15 g, 28.6 mmol) and im-a-9 (11.1 g, 30 mmol) were added to 300 ml of THF, and the mixture was stirred and refluxed. Then, potassium carbonate (11.8 g, 85.7 mmol) was dissolved in 100 ml of water and added thereto, and the mixture was sufficiently stirred and bis(tri-tert-butylphosphine)palladium(0) (0.1 g, 0.3 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.6 g of Compound 18. (Yield: 70%, MS: [M+H]+=732)

### Synthesis Example 19

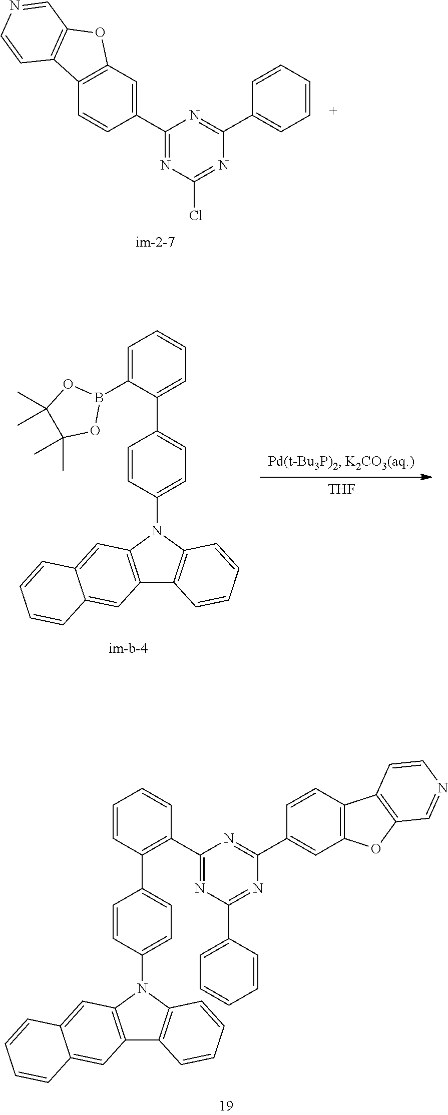 [SMILES: Clc1nc(-c2ccccc2)nc(-c2ccc3c(c2)oc2cnccc23)n1 ; CC1(C)OB(c2ccccc2-c2ccc(-n3c4ccccc4c4cc5ccccc5cc43)cc2)OC1(C)C ; "Pd(t-Bu3P)2, K2CO3(aq.)" ; "THF" ; c1ccc(-c2nc(-c3ccc4c(c3)oc3cnccc34)nc(-c3ccccc3-c3ccc(-n4c5ccccc5c5cc6ccccc6cc54)cc3)n2)cc1]

im-2-7 (15 g, 41.8 mmol) and im-b-4 (21.7 g, 43.9 mmol) were added to 300 ml of THE, and the mixture was stirred and refluxed. Then, potassium carbonate (17.3 g, 125.4 mmol) was dissolved in 100 ml of water and added thereto, and the mixture was sufficiently stirred and bis(tri-tert-butylphosphine)palladium(0) (0.2 g, 0.4 mmol) was added. After the reaction for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 17.6 g of Compound 19. (Yield: 61%, MS: [M+H]+=692)

### Synthesis Example 20

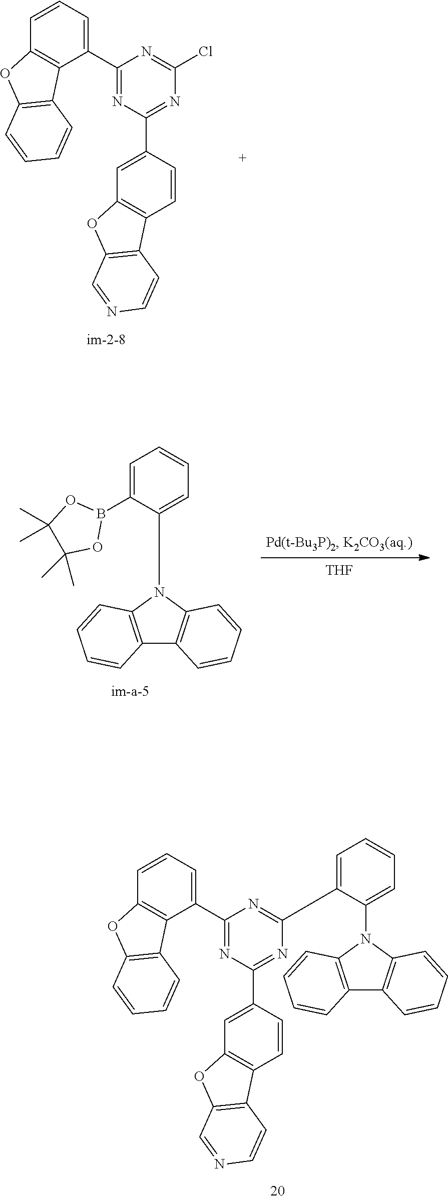 [SMILES: Clc1nc(-c2ccc3c(c2)oc2cnccc23)nc(-c2cccc3oc4ccccc4c23)n1 ; CC1(C)OB(c2ccccc2-n2c3ccccc3c3ccccc32)OC1(C)C ; "Pd(t-Bu3P)2, K2CO3(aq.)" ; "THF" ; c1ccc(-n2c3ccccc3c3ccccc32)c(-c2nc(-c3ccc4c(c3)oc3cnccc34)nc(-c3cccc4oc5ccccc5c34)n2)c1]

im-2-8 (15 g, 33.4 mmol) and im-a-5 (13 g, 35.1 mmol) were added to 300 ml of THF, and the mixture was stirred and refluxed. Then, potassium carbonate (13.9 g, 100.3 mmol) was dissolved in 100 ml of water and added thereto, and the mixture was sufficiently stirred and bis(tri-tert-butylphosphine)palladium(0) (0.2 g, 0.3 mmol) was added. After the reaction for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.7 g of Compound 20. (Yield: 67%, MS: [M+H]+=656)

### Synthesis Example 21

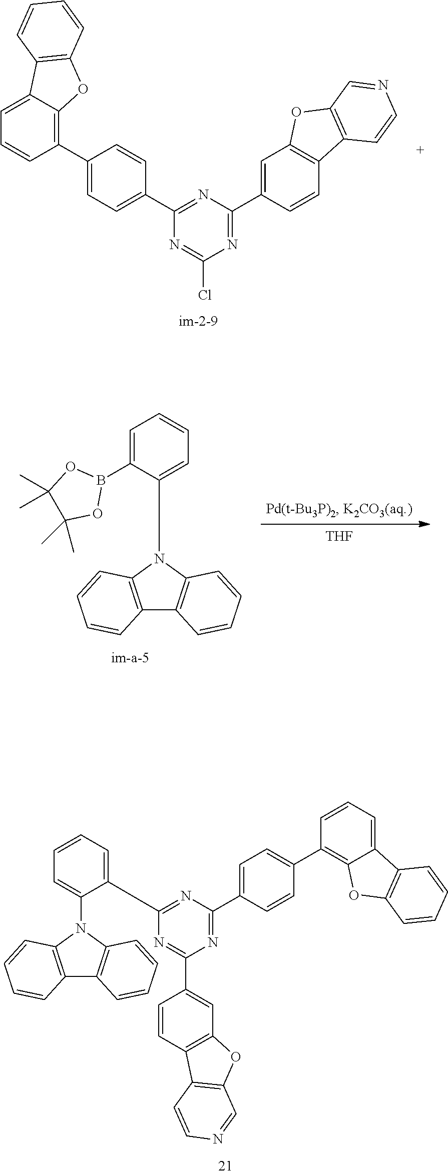 [SMILES: Clc1nc(-c2ccc(-c3cccc4c3oc3ccccc34)cc2)nc(-c2ccc3c(c2)oc2cnccc23)n1 ; CC1(C)OB(c2ccccc2-n2c3ccccc3c3ccccc32)OC1(C)C ; "Pd(t-Bu3P)2, K2CO3(aq.)" ; "THF" ; c1ccc(-n2c3ccccc3c3ccccc32)c(-c2nc(-c3ccc(-c4cccc5c4oc4ccccc45)cc3)nc(-c3ccc4c(c3)oc3cnccc34)n2)c1]

im-2-9 (15 g, 28.6 mmol) and im-a-5 (11.1 g, 30 mmol) were added to 300 ml of THF, and the mixture was stirred and refluxed. Then, potassium carbonate (11.8 g, 85.7 mmol) was dissolved in 100 ml of water and added thereto, and the mixture was sufficiently stirred and bis(tri-tert-butylphosphine)palladium(0) (0.1 g, 0.3 mmol) was added. After the reaction for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15 g of Compound 21. (Yield: 72%, MS: [M+H]+=732)

### Synthesis Example 22

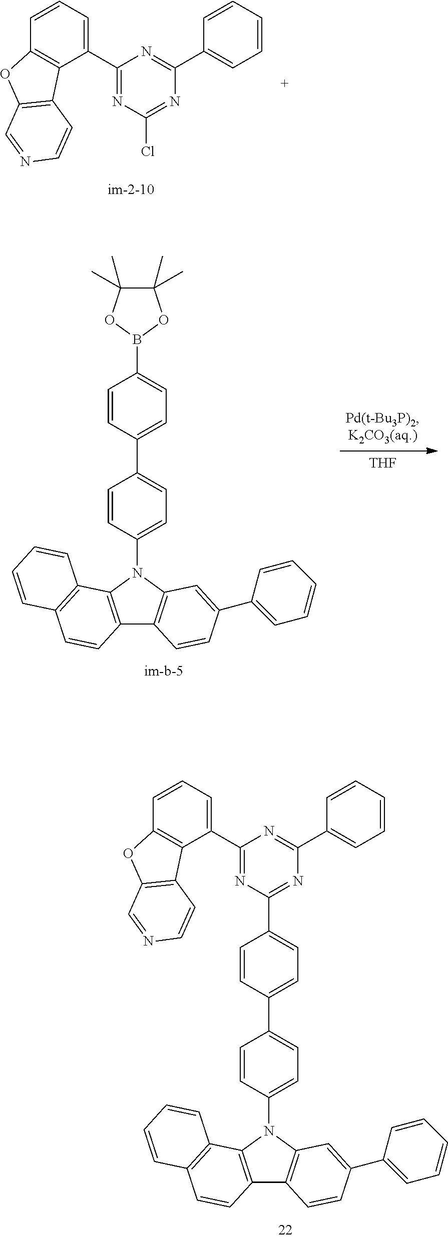 [SMILES: Clc1nc(-c2ccccc2)nc(-c2cccc3oc4cnccc4c23)n1 ; CC1(C)OB(c2ccc(-c3ccc(-n4c5cc(-c6ccccc6)ccc5c5ccc6ccccc6c54)cc3)cc2)OC1(C)C ; "Pd(t-Bu3P)2,K2CO3(aq.)" ; "THF" ; c1ccc(-c2ccc3c4ccc5ccccc5c4n(-c4ccc(-c5ccc(-c6nc(-c7ccccc7)nc(-c7cccc8oc9cnccc9c78)n6)cc5)cc4)c3c2)cc1]

im-2-10 (15 g, 41.8 mmol) and im-b-5 (25.1 g, 43.9 mmol) were added to 300 ml of THF, and the mixture was stirred and refluxed. Then, potassium carbonate (17.3 g, 125.4 mmol) was dissolved in 100 ml of water and added thereto, and the mixture was sufficiently stirred and bis(tri-tert-butylphosphine)palladium(0) (0.2 g, 0.4 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 24.1 g of Compound 22. (Yield: 75%, MS: [M+H]+=768)

### Synthesis Example 23

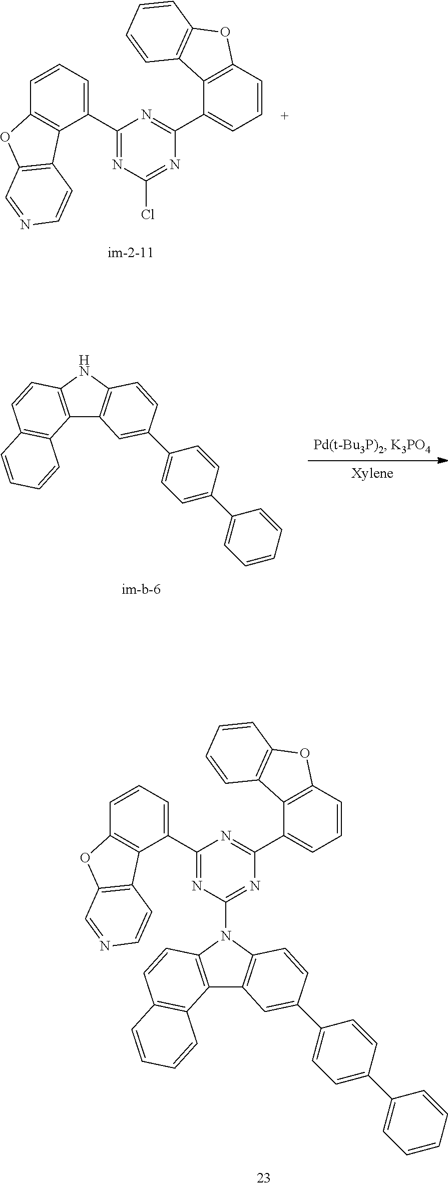

im-2-11 (15 g, 33.4 mmol), im-b-6 (13 g, 35.1 mmol) and potassium phosphate (21.3 g, 100.3 mmol) were added to 300 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 17.2 g of Compound 23. (Yield: 66%, MS: [M+H]+=782)

### Synthesis Example 24

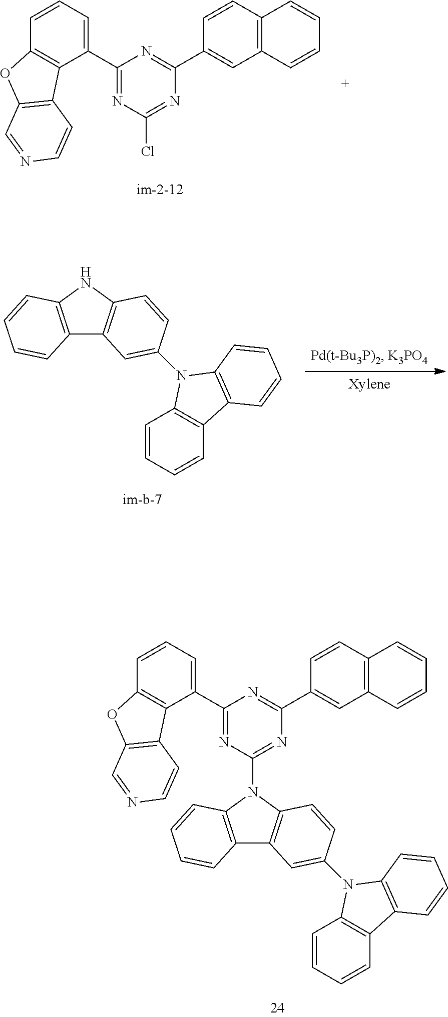

im-2-12 (15 g, 36.7 mmol), im-b-7 (12.8 g, 38.5 mmol) and potassium phosphate (23.4 g, 110.1 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 4 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 17.8 g of Compound 24. (Yield: 69%, MS: [M+H]+=705)

### Synthesis Example 25

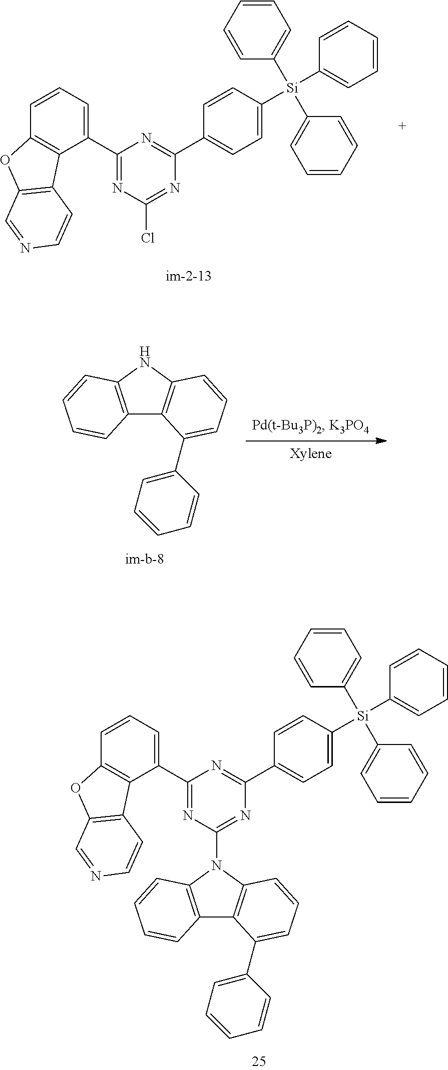

im-2-13 (15 g, 24.3 mmol), im-b-8 (6.2 g, 25.5 mmol) and potassium phosphate (15.5 g, 72.9 mmol) were added to 300 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13 g of Compound 25. (Yield: 65%, MS: [M+H]+=824)

### Synthesis Example 26

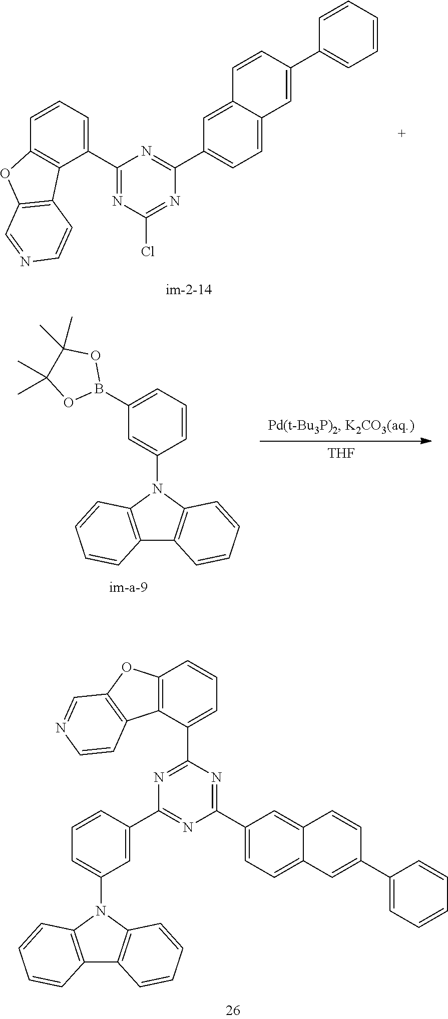 [SMILES: Clc1nc(-c2ccc3cc(-c4ccccc4)ccc3c2)nc(-c2cccc3oc4cnccc4c23)n1 ; CC1(C)OB(c2cccc(-n3c4ccccc4c4ccccc43)c2)OC1(C)C ; "Pd(t-Bu3P)2, K2CO3(aq.)" ; "THF" ; c1ccc(-c2ccc3cc(-c4nc(-c5cccc(-n6c7ccccc7c7ccccc76)c5)nc(-c5cccc6oc7cnccc7c56)n4)ccc3c2)cc1]

im-2-14 (15 g, 30.9 mmol) and im-a-9 (12 g, 32.5 mmol) were added to 300 ml of THE, and the mixture was stirred and refluxed. Then, potassium carbonate (12.8 g, 92.8 mmol) was dissolved in 100 ml of water and added thereto, and the mixture was sufficiently stirred and bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.5 g of Compound 26. (Yield: 63%, MS: [M+H]+=692)

### Synthesis Example 27

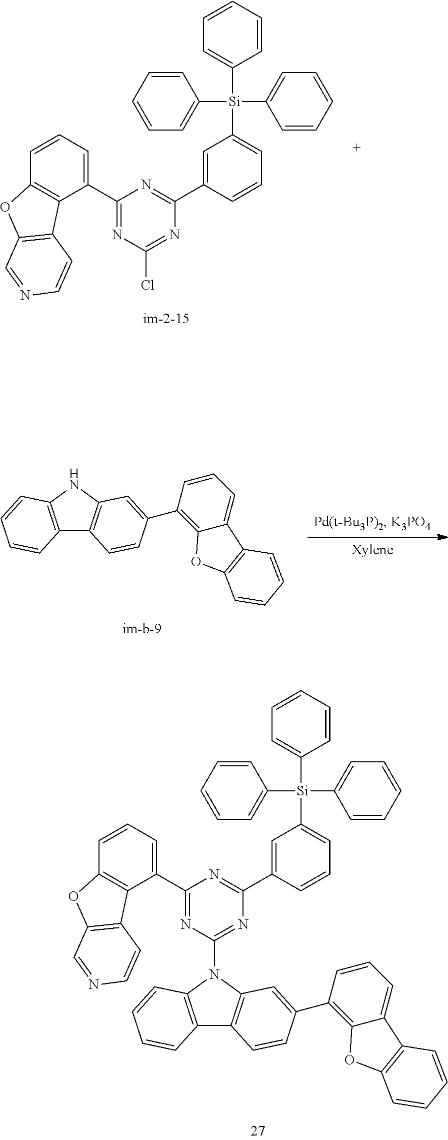

im-2-15 (15 g, 24.3 mmol), im-b-9 (8.5 g, 25.5 mmol) and potassium phosphate (15.5 g, 72.9 mmol) were added to 300 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 14.9 g of Compound 27. (Yield: 67%, MS: [M+H]+=914)

### Synthesis Example 28

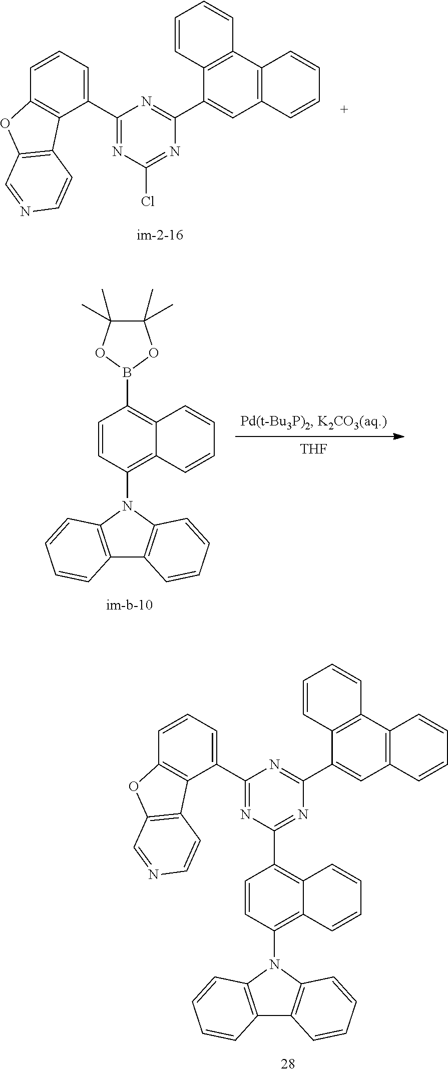 [SMILES: Clc1nc(-c2cc3ccccc3c3ccccc23)nc(-c2cccc3oc4cnccc4c23)n1 ; CC1(C)OB(c2ccc(-n3c4ccccc4c4ccccc43)c3ccccc23)OC1(C)C ; "Pd(t-Bu3P)2, K2CO3(aq.)" ; "THF" ; c1ccc2c(c1)cc(-c1nc(-c3ccc(-n4c5ccccc5c5ccccc54)c4ccccc34)nc(-c3cccc4oc5cnccc5c34)n1)c1ccccc12]

im-2-16 (15 g, 32.7 mmol) and im-b-10 (14.4 g, 34.3 mmol) were added to 300 ml of THF, and the mixture was stirred and refluxed. Then, potassium carbonate (13.6 g, 98.1 mmol) was dissolved in 100 ml of water and added thereto, and the mixture was sufficiently stirred and bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.3 g of Compound 28. (Yield: 61%, MS: [M+H]+=716)

### Synthesis Example 29

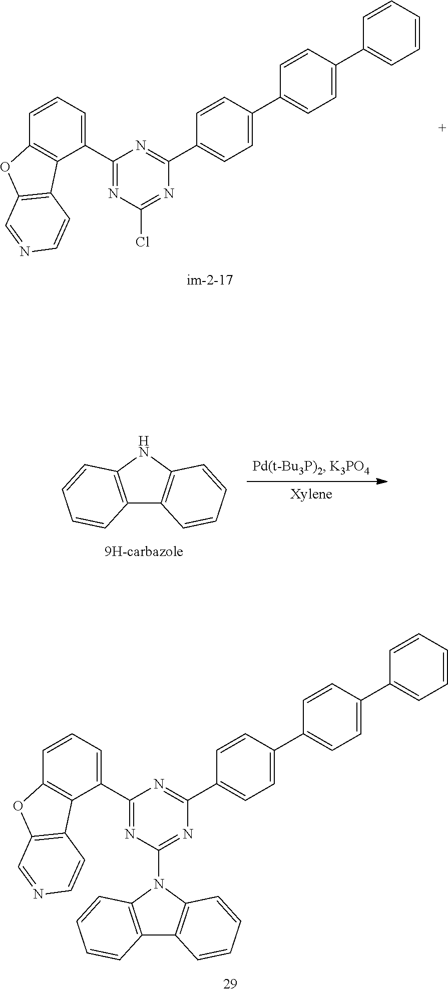

im-2-17 (15 g, 29.4 mmol), 9H-carbazole (5.2 g, 30.8 mmol) and potassium phosphate (18.7 g, 88.1 mmol) were added to 300 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.4 g of Compound 29. (Yield: 66%, MS: [M+H]+=642)

### Synthesis Example 30

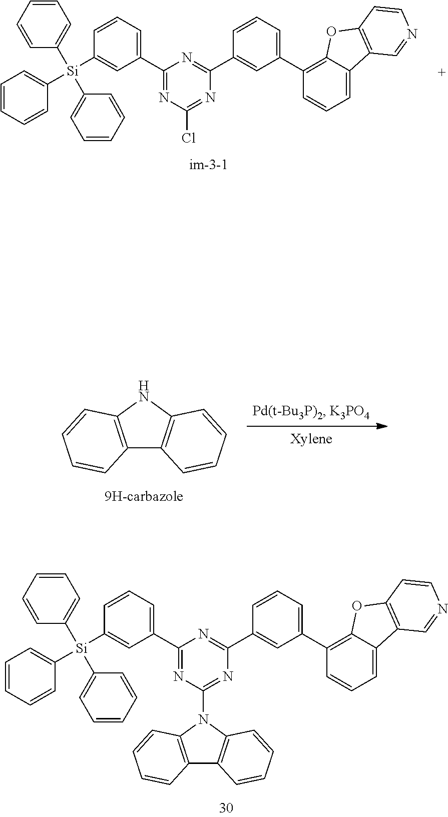

im-3-1 (15 g, 21.6 mmol), 9H-carbazole (3.8 g, 22.7 mmol) and potassium phosphate (13.8 g, 64.9 mmol) were added to 300 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13 g of Compound 30. (Yield: 73%, MS: [M+H]+=824)

### Synthesis Example 31

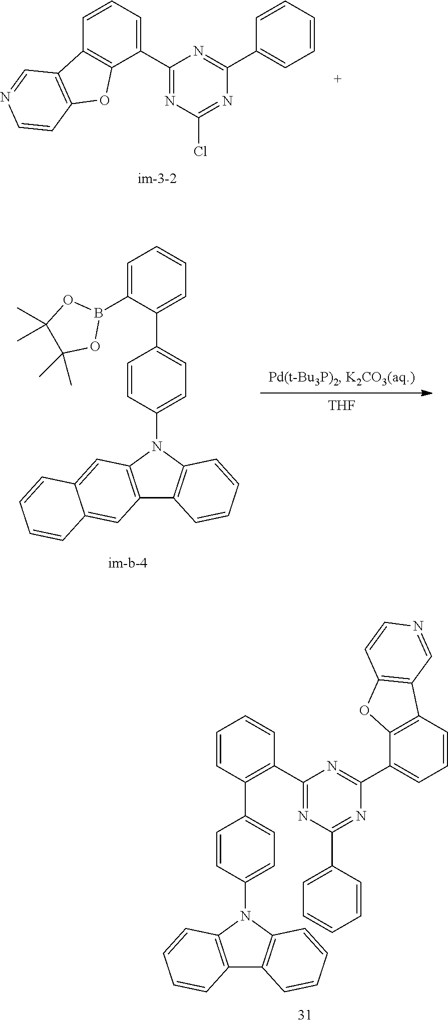 [SMILES: Clc1nc(-c2ccccc2)nc(-c2cccc3c2oc2ccncc23)n1 ; CC1(C)OB(c2ccccc2-c2ccc(-n3c4ccccc4c4cc5ccccc5cc43)cc2)OC1(C)C ; "Pd(t-Bu3P)2, K2CO3(aq.)" ; "THF" ; c1ccc(-c2nc(-c3ccccc3-c3ccc(-n4c5ccccc5c5ccccc54)cc3)nc(-c3cccc4c3oc3ccncc34)n2)cc1]

im-3-2 (15 g, 41.8 mmol) and im-b-4 (21.7 g, 43.9 mmol) were added to 300 ml of THF, and the mixture was stirred and refluxed. Then, potassium carbonate (17.3 g, 125.4 mmol) was dissolved in 100 ml of water and added thereto, and the mixture was sufficiently stirred and bis(tri-tert-butylphosphine)palladium(0) (0.2 g, 0.4 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 17.4 g of Compound 31. (Yield: 65%, MS: [M+H]+=642)

### Synthesis Example 32

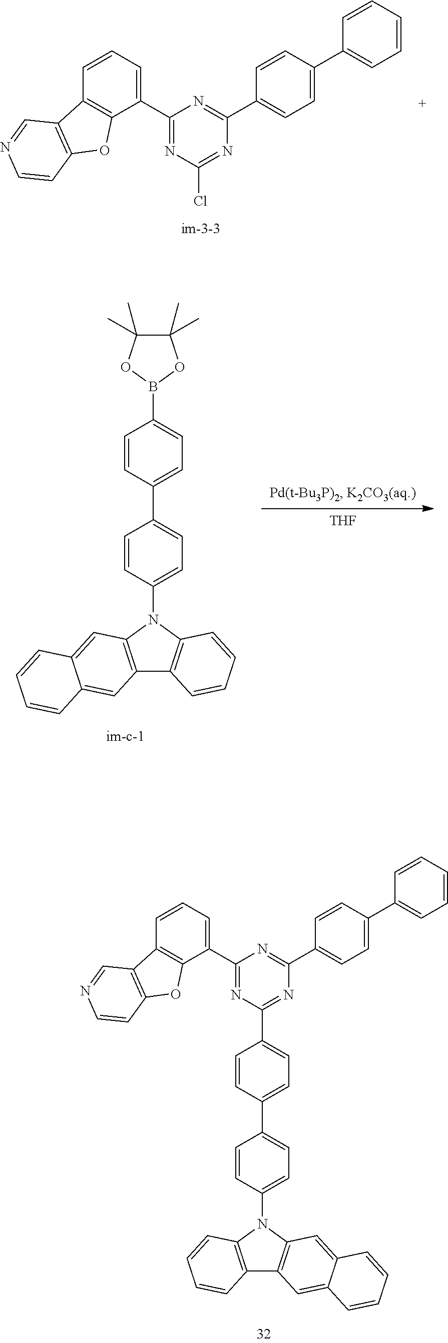 [SMILES: Clc1nc(-c2ccc(-c3ccccc3)cc2)nc(-c2cccc3c2oc2ccncc23)n1 ; CC1(C)OB(c2ccc(-c3ccc(-n4c5ccccc5c5cc6ccccc6cc54)cc3)cc2)OC1(C)C ; "Pd(t-Bu3P)2, K2CO3(aq.)" ; "THF" ; c1ccc(-c2ccc(-c3nc(-c4ccc(-c5ccc(-n6c7ccccc7c7cc8ccccc8cc76)cc5)cc4)nc(-c4cccc5c4oc4ccncc45)n3)cc2)cc1]

im-3-3 (15 g, 34.5 mmol) and im-c-1 (17.9 g, 36.2 mmol) were added to 300 ml of THF, and the mixture was stirred and refluxed. Then, potassium carbonate (14.3 g, 103.5 mmol) was dissolved in 100 ml of water and added thereto, and the mixture was sufficiently stirred and bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 19.3 g of Compound 32. (Yield: 73%, MS: [M+H]+=768)

### Synthesis Example 33

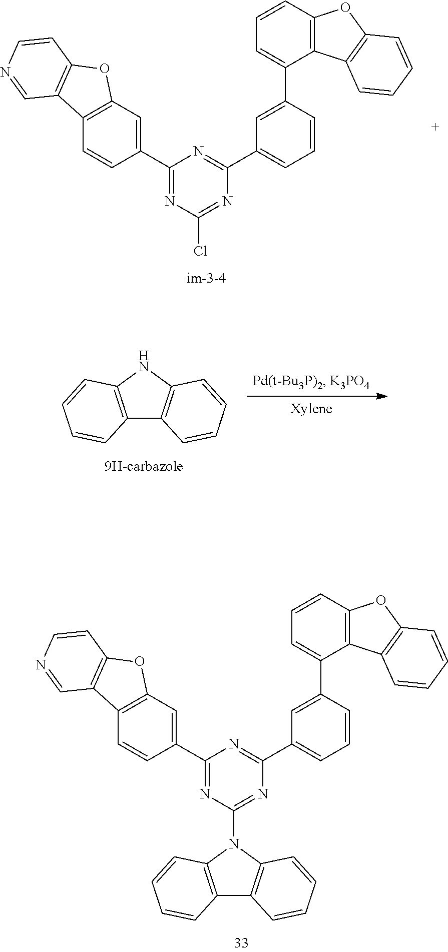

im-3-4 (15 g, 28.6 mmol), 9H-carbazole (5 g, 30 mmol) and potassium phosphate (18.2 g, 85.7 mmol) were added to 300 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.4 g of Compound 33. (Yield: 66%, MS: [M+H]+=656)

### Synthesis Example 34

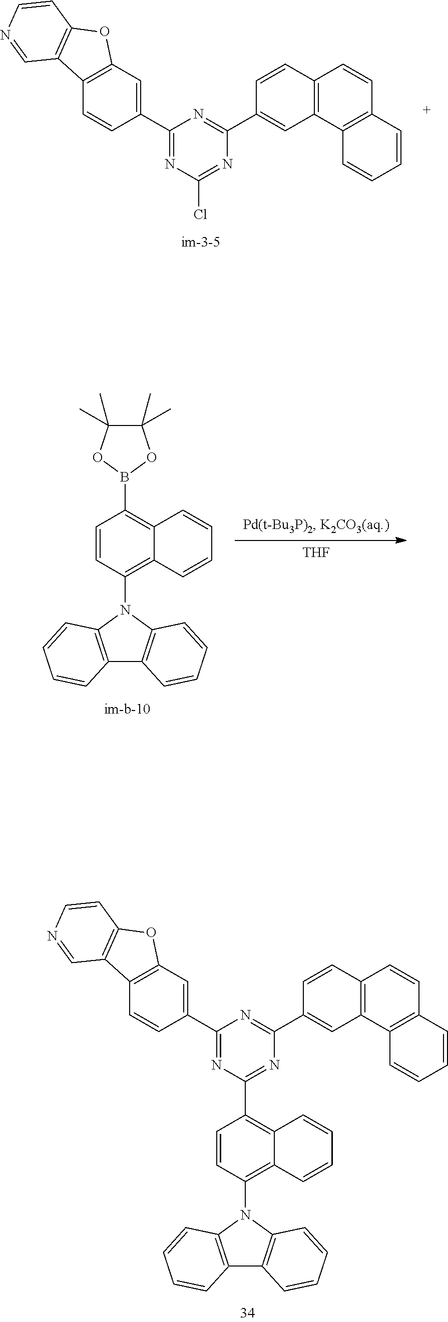 [SMILES: Clc1nc(-c2ccc3c(c2)oc2ccncc23)nc(-c2ccc3ccc4ccccc4c3c2)n1 ; CC1(C)OB(c2ccc(-n3c4ccccc4c4ccccc43)c3ccccc23)OC1(C)C ; "Pd(t-Bu3P)2, K2CO3(aq.)" ; "THF" ; c1ccc2c(c1)ccc1ccc(-c3nc(-c4ccc5c(c4)oc4ccncc45)nc(-c4ccc(-n5c6ccccc6c6ccccc65)c5ccccc45)n3)cc12]

im-3-5 (15 g, 32.7 mmol) and im-b-10 (14.4 g, 34.3 mmol) were added to 300 ml of THF, and the mixture was stirred and refluxed. Then, potassium carbonate (13.6 g, 98.1 mmol) was dissolved in 100 ml of water and added thereto, and the mixture was sufficiently stirred and bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.9 g of Compound 34. (Yield: 68%, MS: [M+H]+=716)

### Synthesis Example 35

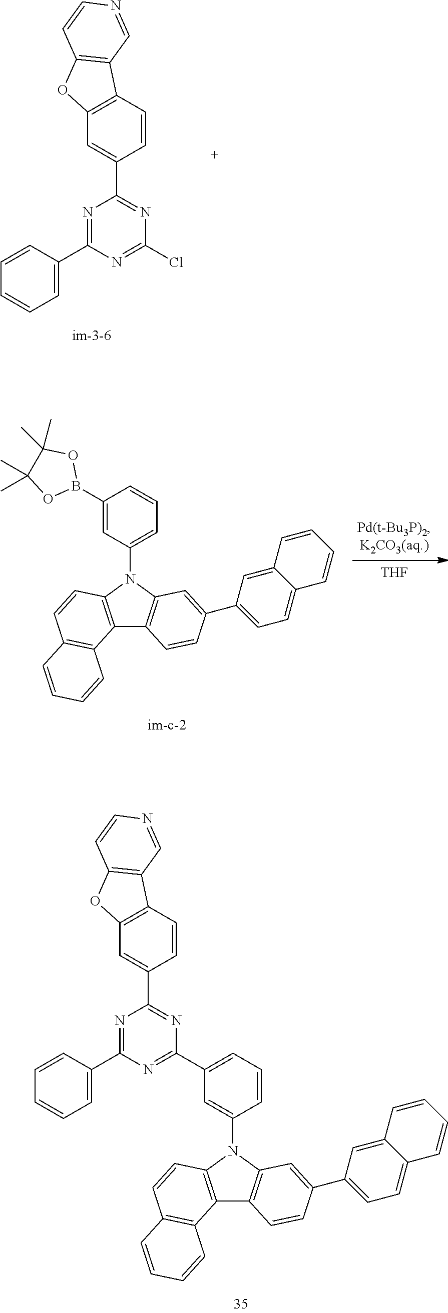 [SMILES: Clc1nc(-c2ccccc2)nc(-c2ccc3c(c2)oc2ccncc23)n1 ; CC1(C)OB(c2cccc(-n3c4cc(-c5ccc6ccccc6c5)ccc4c4c5ccccc5ccc43)c2)OC1(C)C ; "Pd(t-Bu3P)2,K2CO3(aq.)" ; "THF" ; c1ccc(-c2nc(-c3cccc(-n4c5cc(-c6ccc7ccccc7c6)ccc5c5c6ccccc6ccc54)c3)nc(-c3ccc4c(c3)oc3ccncc34)n2)cc1]

im-3-6 (15 g, 41.8 mmol) and im-c-2 (23.9 g, 43.9 mmol) were added to 300 ml of THE, and the mixture was stirred and refluxed. Then, potassium carbonate (17.3 g, 125.4 mmol) was dissolved in 100 ml of water and added thereto, and the mixture was sufficiently stirred and bis(tri-tert-butylphosphine)palladium(0) (0.2 g, 0.4 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 19.2 g of Compound 35. (Yield: 62%, MS: [M+H]+=742)

### Synthesis Example 36

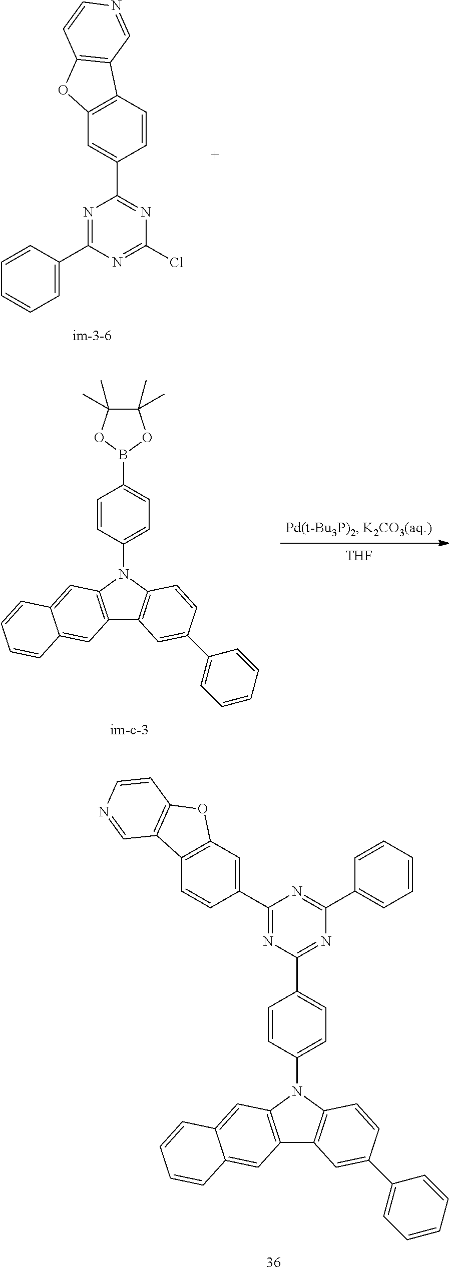 [SMILES: Clc1nc(-c2ccccc2)nc(-c2ccc3c(c2)oc2ccncc23)n1 ; CC1(C)OB(c2ccc(-n3c4ccc(-c5ccccc5)cc4c4cc5ccccc5cc43)cc2)OC1(C)C ; "Pd(t-Bu3P)2, K2CO3(aq.)" ; "THF" ; c1ccc(-c2ccc3c(c2)c2cc4ccccc4cc2n3-c2ccc(-c3nc(-c4ccccc4)nc(-c4ccc5c(c4)oc4ccncc45)n3)cc2)cc1]

im-3-6 (15 g, 41.8 mmol) and im-c-3 (21.7 g, 43.9 mmol) were added to 300 ml of THF, and the mixture was stirred and refluxed. Then, potassium carbonate (17.3 g, 125.4 mmol) was dissolved in 100 ml of water and added thereto, and the mixture was sufficiently stirred and bis(tri-tert-butylphosphine)palladium(0) (0.2 g, 0.4 mmol) was added. After the reaction for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 18.2 g of Compound 36. (Yield: 63%, MS: [M+H]+=692)

### Synthesis Example 37

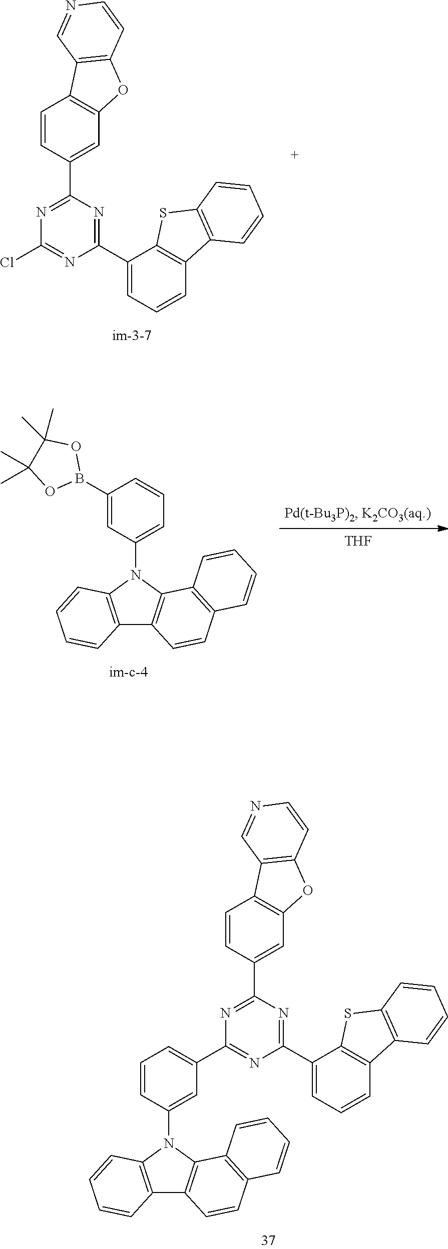 [SMILES: Clc1nc(-c2ccc3c(c2)oc2ccncc23)nc(-c2cccc3c2sc2ccccc23)n1 ; CC1(C)OB(c2cccc(-n3c4ccccc4c4ccc5ccccc5c43)c2)OC1(C)C ; "Pd(t-Bu3P)2, K2CO3(aq.)" ; "THF" ; c1cc(-c2nc(-c3ccc4c(c3)oc3ccncc34)nc(-c3cccc4c3sc3ccccc34)n2)cc(-n2c3ccccc3c3ccc4ccccc4c32)c1]

im-3-7 (15 g, 32.3 mmol) and im-c-4 (16.8 g, 33.9 mmol) were added to 300 ml of THF, and the mixture was stirred and refluxed. Then, potassium carbonate (13.4 g, 96.8 mmol) was dissolved in 100 ml of water and added thereto, and the mixture was sufficiently stirred and bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.9 g of Compound 37. (Yield: 64%, MS: [M+H]+=722)

### Synthesis Example 38

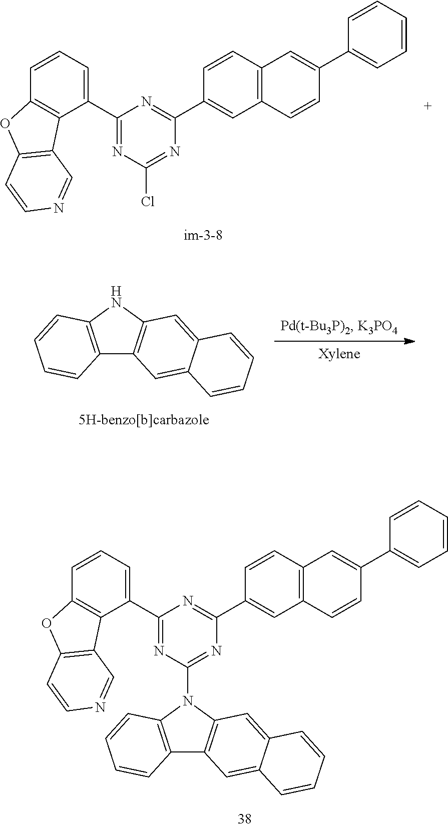

im-3-8 (15 g, 30.9 mmol), 5H-benzo[b]carbazole (7.1 g, 32.5 mmol) and potassium phosphate (19.7 g, 92.8 mmol) were added to 300 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 4 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 14 g of Compound 38. (Yield: 68%, MS: [M+H]+=666)

### Synthesis Example 39

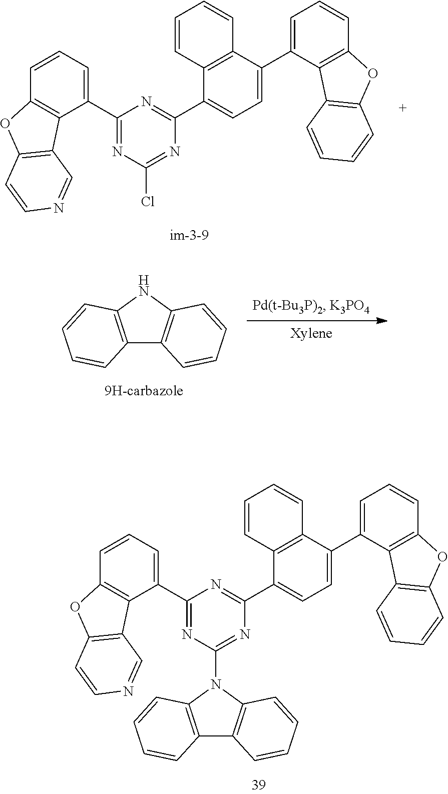

im-3-9 (15 g, 26.1 mmol), 9H-carbazole (4.6 g, 27.4 mmol) and potassium phosphate (16.6 g, 78.3 mmol) were added to 300 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.8 g of Compound 39. (Yield: 64%, MS: [M+H]+=706)

### Synthesis Example 40

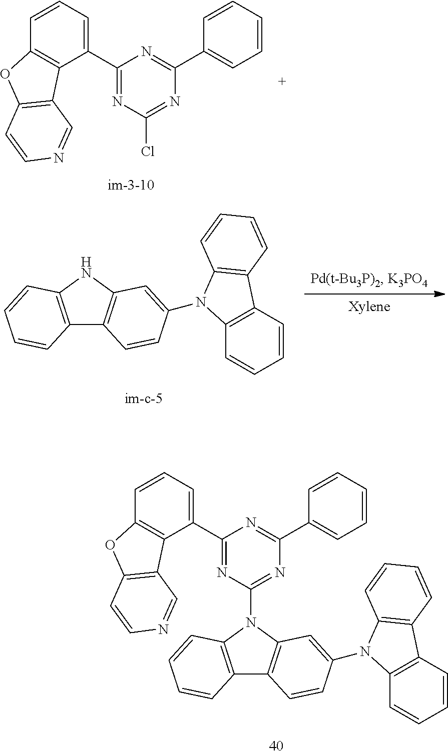

im-3-10 (15 g, 41.8 mmol), im-c-5 (14.6 g, 43.9 mmol) and potassium phosphate (26.6 g, 125.4 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 19.7 g of Compound 40. (Yield: 72%, MS: [M+H]+=655)

### Synthesis Example 41

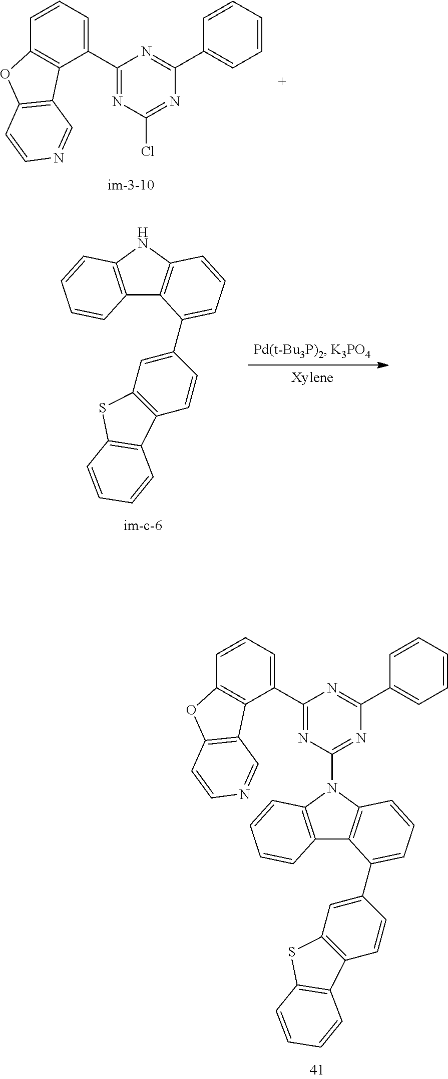

im-3-10 (15 g, 41.8 mmol), im-c-6 (15.3 g, 43.9 mmol) and potassium phosphate (26.6 g, 125.4 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 4 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 17.4 g of Compound 41. (Yield: 62%, MS: [M+H]+=672)

### Synthesis Example 42

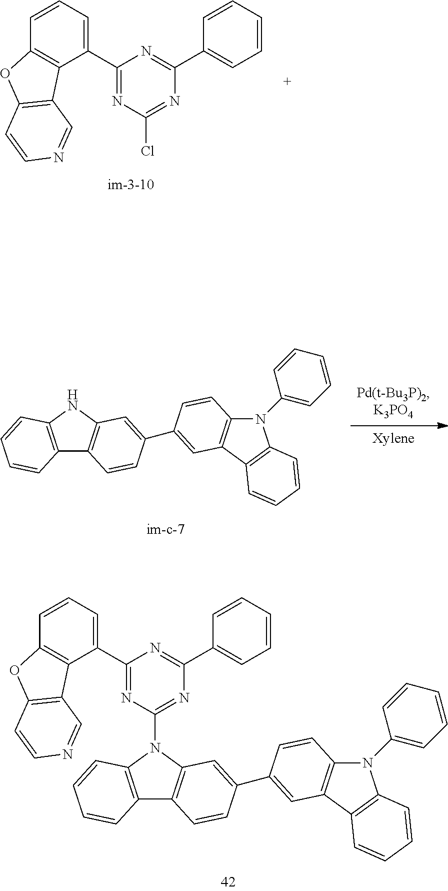

im-3-10 (15 g, 41.8 mmol), im-c-7 (17.9 g, 43.9 mmol) and potassium phosphate (26.6 g, 125.4 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 4 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 22.9 g of Compound 42. (Yield: 75%, MS: [M+H]+=731)

### Synthesis Example 43

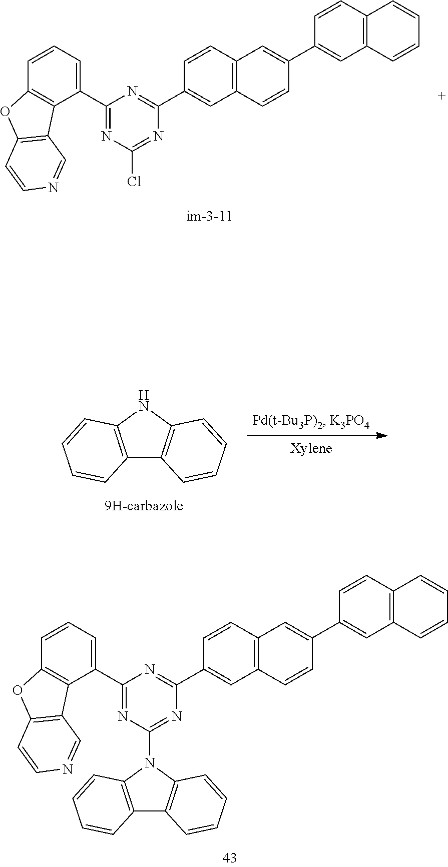

im-3-11 (15 g, 28 mmol), 9H-carbazole (4.9 g, 29.4 mmol) and potassium phosphate (17.9 g, 84.1 mmol) were added to 300 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 4 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13.2 g of Compound 43. (Yield: 71%, MS: [M+H]+=666)

### Synthesis Example 44

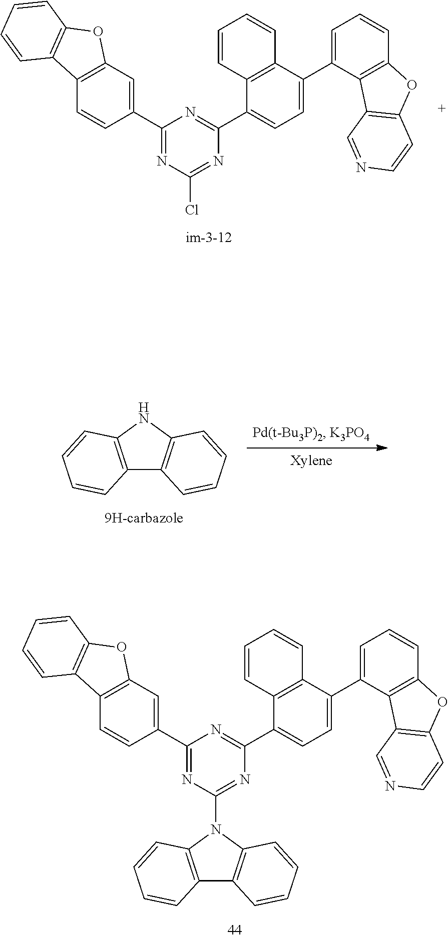

im-3-12 (15 g, 26.1 mmol), 9H-carbazole (4.6 g, 27.4 mmol) and potassium phosphate (16.6 g, 78.3 mmol) were added to 300 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12 g of Compound 44. (Yield: 65%, MS: [M+H]+=706)

### Synthesis Example 45

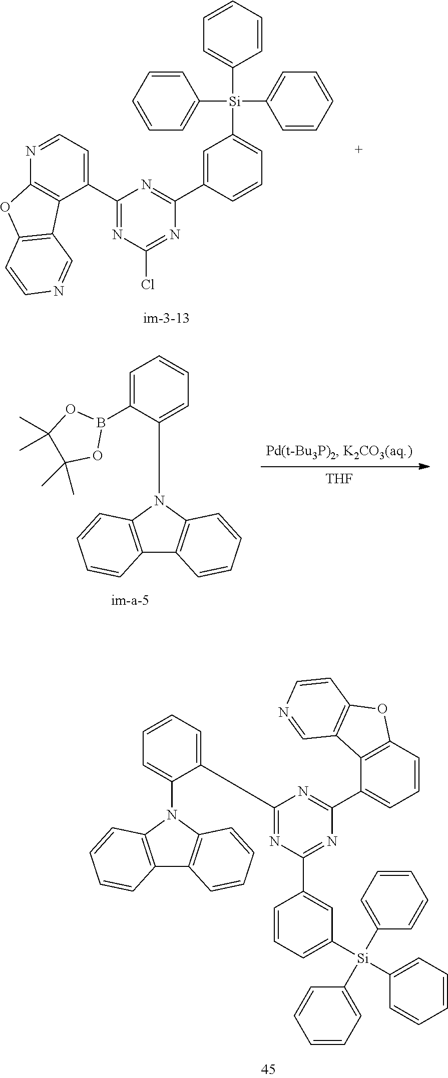 [SMILES: Clc1nc(-c2cccc([Si](c3ccccc3)(c3ccccc3)c3ccccc3)c2)nc(-c2ccnc3oc4ccncc4c23)n1 ; CC1(C)OB(c2ccccc2-n2c3ccccc3c3ccccc32)OC1(C)C ; "Pd(t-Bu3P)2, K2CO3(aq.)" ; "THF" ; c1ccc([Si](c2ccccc2)(c2ccccc2)c2cccc(-c3nc(-c4ccccc4-n4c5ccccc5c5ccccc54)nc(-c4cccc5oc6ccncc6c45)n3)c2)cc1]

im-3-13 (15 g, 24.3 mmol) and im-a-5 (9.4 g, 25.5 mmol) were added to 300 ml of THF, and the mixture was stirred and refluxed. Then, potassium carbonate (10.1 g, 72.9 mmol) was dissolved in 100 ml of water and added thereto, and the mixture was sufficiently stirred and bis(tri-tert-butylphosphine)palladium(0) (0.1 g, 0.2 mmol) was added. After the reaction for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.6 g of Compound 45. (Yield: 68%, MS: [M+H]+=824)

### Synthesis Example 46

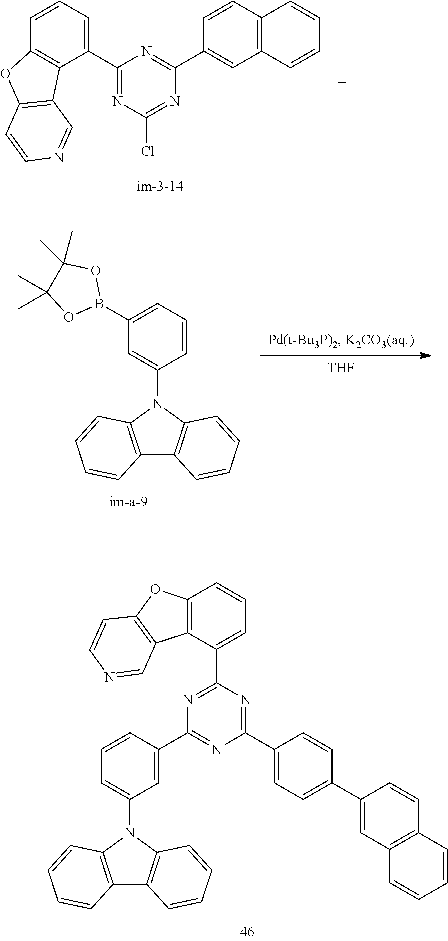 [SMILES: Clc1nc(-c2ccc3ccccc3c2)nc(-c2cccc3oc4ccncc4c23)n1 ; CC1(C)OB(c2cccc(-n3c4ccccc4c4ccccc43)c2)OC1(C)C ; "Pd(t-Bu3P)2, K2CO3(aq.)" ; "THF" ; c1cc(-c2nc(-c3ccc(-c4ccc5ccccc5c4)cc3)nc(-c3cccc4oc5ccncc5c34)n2)cc(-n2c3ccccc3c3ccccc32)c1]

im-3-14 (15 g, 30.9 mmol) and im-a-9 (12 g, 32.5 mmol) were added to 300 ml of THE, and the mixture was stirred and refluxed. Then, potassium carbonate (12.8 g, 92.8 mmol) was dissolved in 100 ml of water and added thereto, and the mixture was sufficiently stirred and bis(tri-tert-butylphosphine)palladium(0) (0.2 g, 0.3 mmol) was added. After the reaction for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.3 g of Compound 46. (Yield: 62%, MS: [M+H]+=692)

### Synthesis Example 47

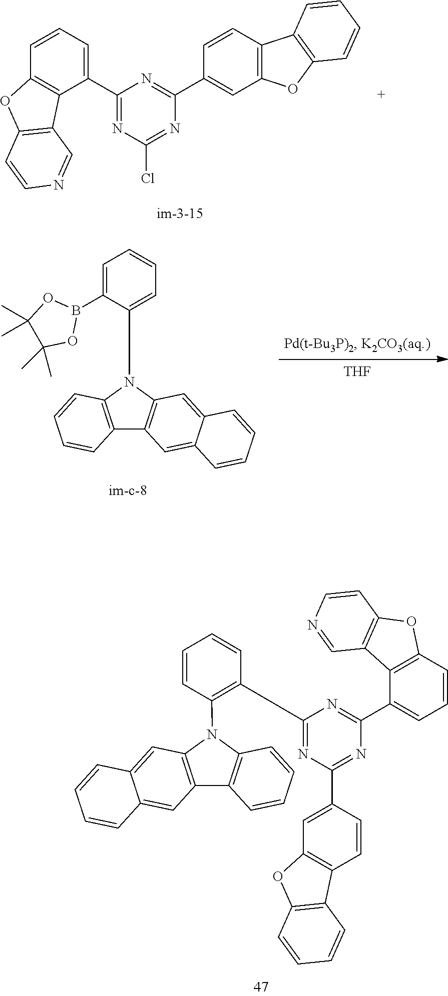 [SMILES: Clc1nc(-c2ccc3c(c2)oc2ccccc23)nc(-c2cccc3oc4ccncc4c23)n1 ; CC1(C)OB(c2ccccc2-n2c3ccccc3c3cc4ccccc4cc32)OC1(C)C ; "Pd(t-Bu3P)2, K2CO3(aq.)" ; "THF" ; c1ccc(-n2c3ccccc3c3cc4ccccc4cc32)c(-c2nc(-c3ccc4c(c3)oc3ccccc34)nc(-c3cccc4oc5ccncc5c34)n2)c1]

im-3-15 (15 g, 33.4 mmol) and im-c-8 (14.7 g, 35.1 mmol) were added to 300 ml of THF, and the mixture was stirred and refluxed. Then, potassium carbonate (13.9 g, 100.3 mmol) was dissolved in 100 ml of water and added thereto, and the mixture was sufficiently stirred and bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.1 g of Compound 47. (Yield: 64%, MS: [M+H]+=706)

### Synthesis Example 48

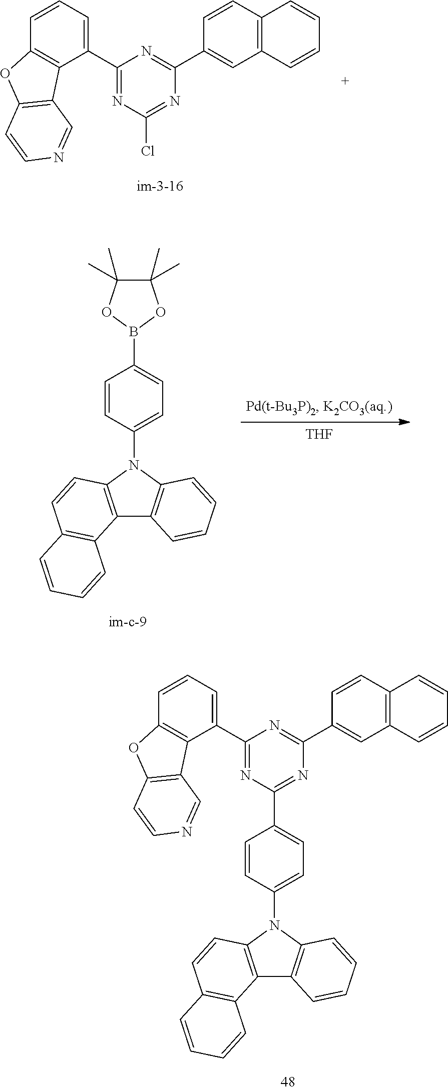 [SMILES: Clc1nc(-c2ccc3ccccc3c2)nc(-c2cccc3oc4ccncc4c23)n1 ; CC1(C)OB(c2ccc(-n3c4ccccc4c4c5ccccc5ccc43)cc2)OC1(C)C ; "Pd(t-Bu3P)2, K2CO3(aq.)" ; "THF" ; c1ccc2cc(-c3nc(-c4ccc(-n5c6ccccc6c6c7ccccc7ccc65)cc4)nc(-c4cccc5oc6ccncc6c45)n3)ccc2c1]

im-3-16 (15 g, 36.7 mmol) and im-c-9 (16.2 g, 38.5 mmol) were added to 300 ml of THF, and the mixture was stirred and refluxed. Then, potassium carbonate (15.2 g, 110.1 mmol) was dissolved in 100 ml of water and added thereto, and the mixture was sufficiently stirred and bis(tri-tert-butylphosphine)palladium(0) (0.2 g, 0.4 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 17.8 g of Compound 48. (Yield: 73%, MS: [M+H]+=666)

### Synthesis Example 49

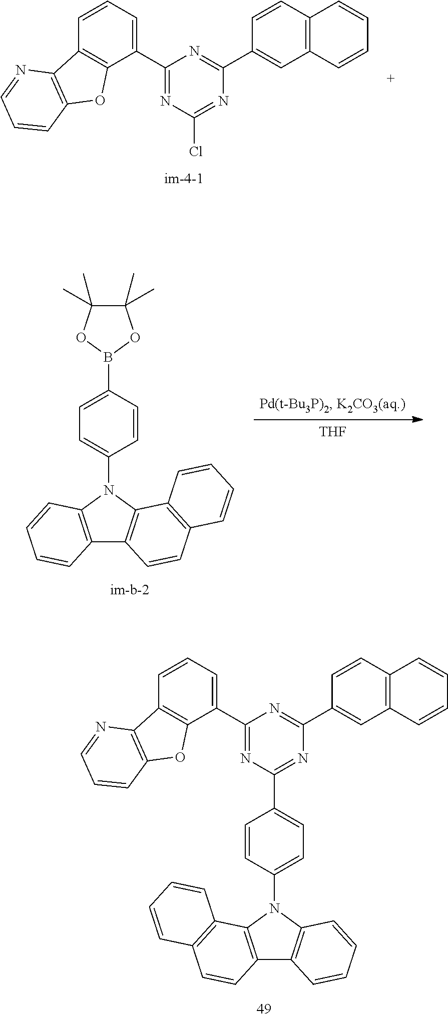 [SMILES: Clc1nc(-c2ccc3ccccc3c2)nc(-c2cccc3c2oc2cccnc23)n1 ; CC1(C)OB(c2ccc(-n3c4ccccc4c4ccc5ccccc5c43)cc2)OC1(C)C ; "Pd(t-Bu3P)2, K2CO3(aq.)" ; "THF" ; c1ccc2cc(-c3nc(-c4ccc(-n5c6ccccc6c6ccc7ccccc7c65)cc4)nc(-c4cccc5c4oc4cccnc45)n3)ccc2c1]

im-4-1 (15 g, 36.7 mmol) and im-b-2 (16.2 g, 38.5 mmol) were added to 300 ml of THF, and the mixture was stirred and refluxed. Then, potassium carbonate (15.2 g, 110.1 mmol) was dissolved in 100 ml of water and added thereto, and the mixture was sufficiently stirred and bis(tri-tert-butylphosphine)palladium(0) (0.2 g, 0.4 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 18.1 g of Compound 49. (Yield: 74%, MS: [M+H]+=666)

### Synthesis Example 50

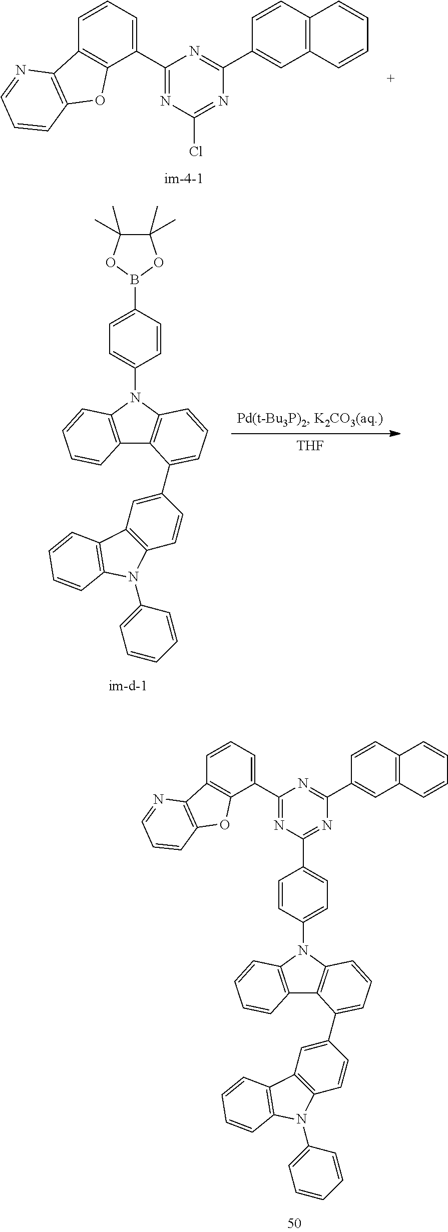 [SMILES: Clc1nc(-c2ccc3ccccc3c2)nc(-c2cccc3c2oc2cccnc23)n1 ; CC1(C)OB(c2ccc(-n3c4ccccc4c4c(-c5ccc6c(c5)c5ccccc5n6-c5ccccc5)cccc43)cc2)OC1(C)C ; "Pd(t-Bu3P)2, K2CO3(aq.)" ; "THF" ; c1ccc(-n2c3ccccc3c3cc(-c4cccc5c4c4ccccc4n5-c4ccc(-c5nc(-c6ccc7ccccc7c6)nc(-c6cccc7c6oc6cccnc67)n5)cc4)ccc32)cc1]

im-4-1 (15 g, 36.7 mmol) and im-d-1 (23.5 g, 38.5 mmol) were added to 300 ml of THF, and the mixture was stirred and refluxed. Then, potassium carbonate (15.2 g, 110.1 mmol) was dissolved in 100 ml of water and added thereto, and the mixture was sufficiently stirred and bis(tri-tert-butylphosphine)palladium(0) (0.2 g, 0.4 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 19.5 g of Compound 50. (Yield: 62%, MS: [M+H]+=857)

### Synthesis Example 51

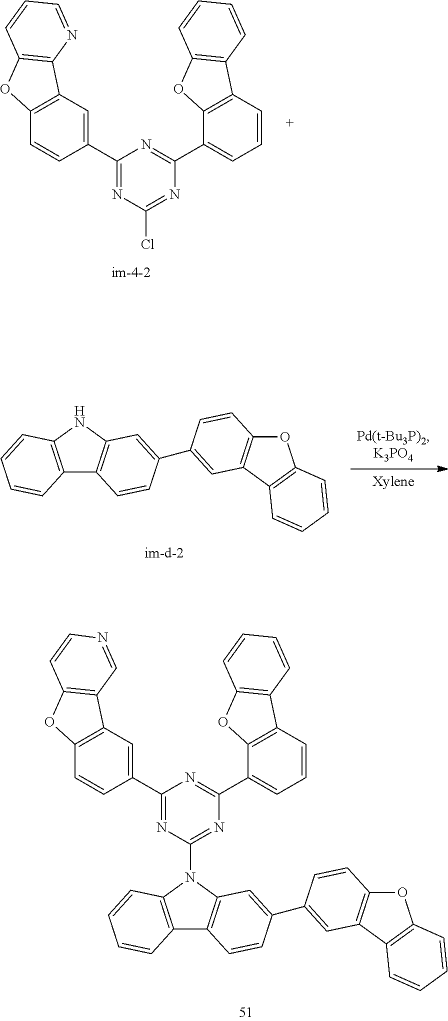

im-4-2 (15 g, 33.4 mmol), im-d-2 (11.7 g, 35.1 mmol) and potassium phosphate (21.3 g, 100.3 mmol) were added to 300 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 16.7 g of Compound 51. (Yield: 67%, MS: [M+H]+=746)

### Synthesis Example 52

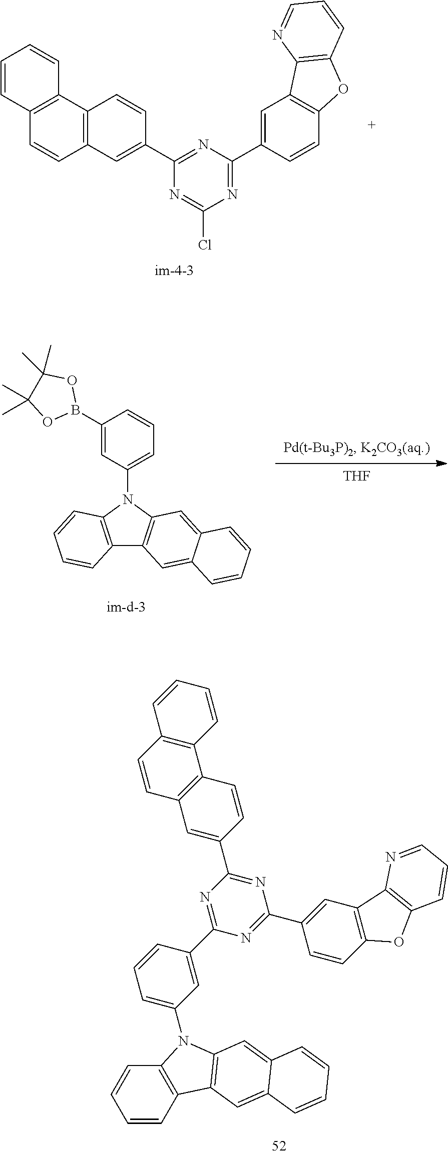 [SMILES: Clc1nc(-c2ccc3c(ccc4ccccc43)c2)nc(-c2ccc3oc4cccnc4c3c2)n1 ; CC1(C)OB(c2cccc(-n3c4ccccc4c4cc5ccccc5cc43)c2)OC1(C)C ; "Pd(t-Bu3P)2, K2CO3(aq.)" ; "THF" ; c1cc(-c2nc(-c3ccc4c(ccc5ccccc54)c3)nc(-c3ccc4oc5cccnc5c4c3)n2)cc(-n2c3ccccc3c3cc4ccccc4cc32)c1]

im-4-3 (15 g, 32.7 mmol) and im-d-3 (14.4 g, 34.3 mmol) were added to 300 ml of THF, and the mixture was stirred and refluxed. Then, potassium carbonate (13.6 g, 98.1 mmol) was dissolved in 100 ml of water and added thereto, and the mixture was sufficiently stirred and bis(tri-tert-butylphosphine)palladium(0) (0.2 g, 0.3 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.2 g of Compound 52. (Yield: 65%, MS: [M+H]+=716)

### Synthesis Example 53

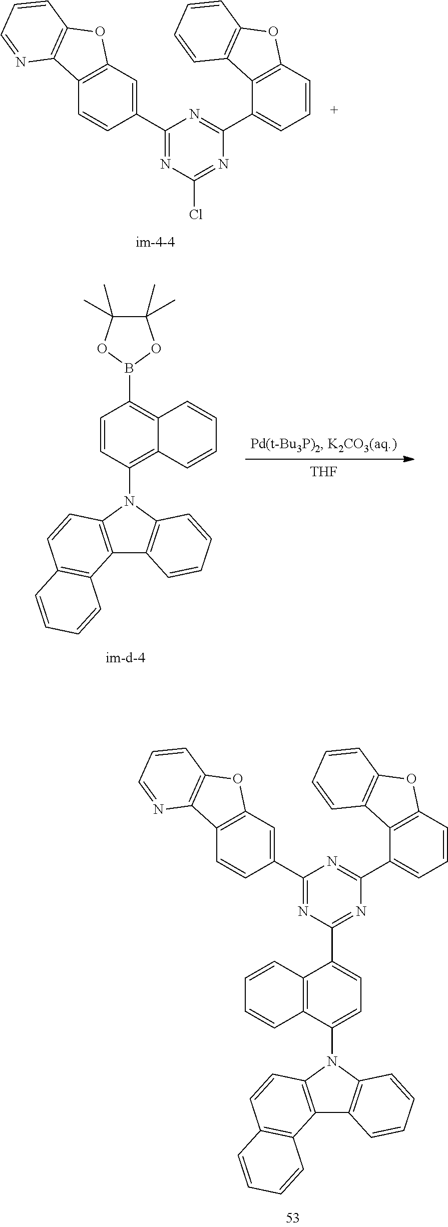 [SMILES: Clc1nc(-c2ccc3c(c2)oc2cccnc23)nc(-c2cccc3oc4ccccc4c23)n1 ; CC1(C)OB(c2ccc(-n3c4ccccc4c4c5ccccc5ccc43)c3ccccc23)OC1(C)C ; "Pd(t-Bu3P)2, K2CO3(aq.)" ; "THF" ; c1ccc2c(c1)ccc1c2c2ccccc2n1-c1ccc(-c2nc(-c3ccc4c(c3)oc3cccnc34)nc(-c3cccc4oc5ccccc5c34)n2)c2ccccc12]

im-4-4 (15 g, 33.4 mmol) and im-d-4 (16.5 g, 35.1 mmol) were added to 300 ml of THF, and the mixture was stirred and refluxed. Then, potassium carbonate (13.9 g, 100.3 mmol) was dissolved in 100 ml of water and added thereto, and the mixture was sufficiently stirred and bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 18.7 g of Compound 53 (Yield: 74%, MS: [M+H]+=756)

### Synthesis Example 54

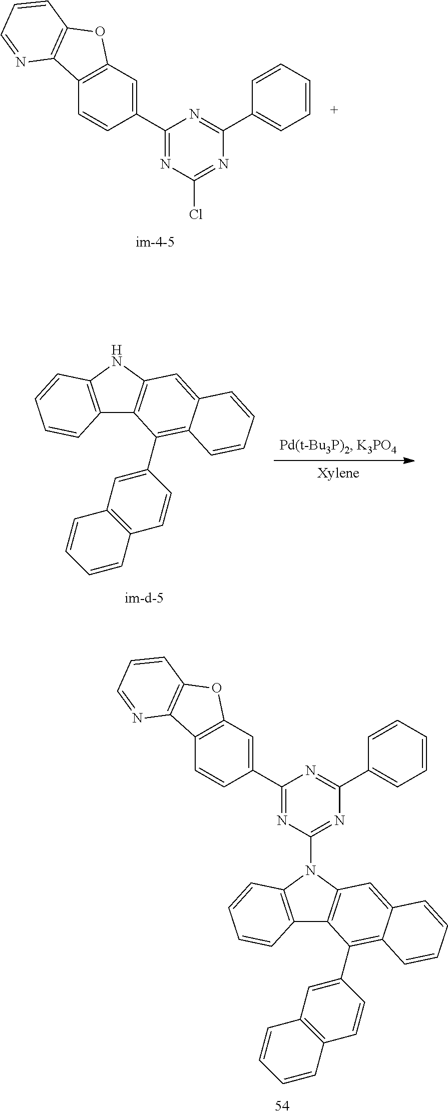

im-4-5 (15 g, 41.8 mmol), im-d-5 (15.1 g, 43.9 mmol) and potassium phosphate (26.6 g, 125.4 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 20.9 g of Compound 54. (Yield: 75%, MS: [M+H]+=666)

### Synthesis Example 55

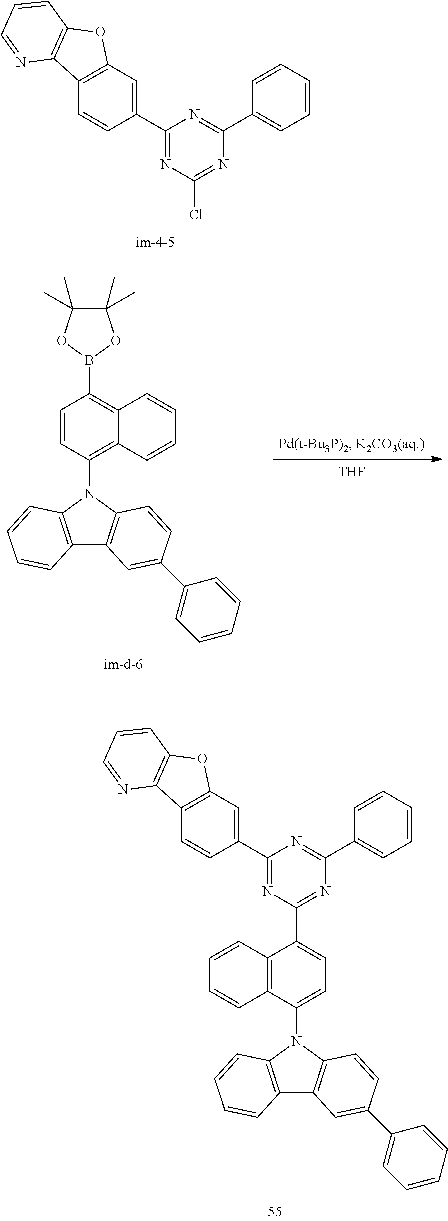 [SMILES: Clc1nc(-c2ccccc2)nc(-c2ccc3c(c2)oc2cccnc23)n1 ; CC1(C)OB(c2ccc(-n3c4ccccc4c4cc(-c5ccccc5)ccc43)c3ccccc23)OC1(C)C ; "Pd(t-Bu3P)2, K2CO3(aq.)" ; "THF" ; c1ccc(-c2ccc3c(c2)c2ccccc2n3-c2ccc(-c3nc(-c4ccccc4)nc(-c4ccc5c(c4)oc4cccnc45)n3)c3ccccc23)cc1]

im-4-5 (15 g, 41.8 mmol) and im-d-6 (21.7 g, 43.9 mmol) were added to 300 ml of THE, and the mixture was stirred and refluxed. Then, potassium carbonate (17.3 g, 125.4 mmol) was dissolved in 100 ml of water and added thereto, and the mixture was sufficiently stirred and bis(tri-tert-butylphosphine)palladium(0) (0.2 g, 0.4 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 21.4 g of Compound 55. (Yield: 74%, MS: [M+H]+=692)

### Synthesis Example 56

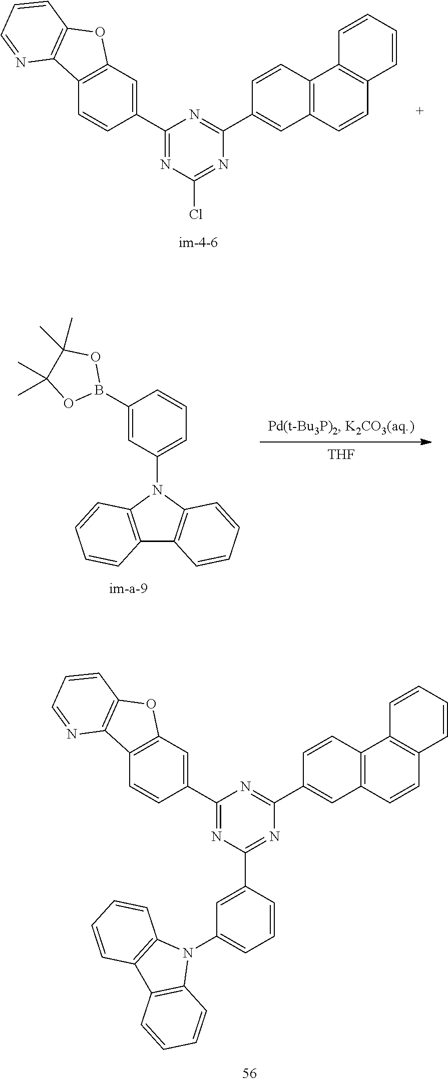 [SMILES: Clc1nc(-c2ccc3c(ccc4ccccc43)c2)nc(-c2ccc3c(c2)oc2cccnc23)n1 ; CC1(C)OB(c2cccc(-n3c4ccccc4c4ccccc43)c2)OC1(C)C ; "Pd(t-Bu3P)2, K2CO3(aq.)" ; "THF" ; c1cc(-c2nc(-c3ccc4c(ccc5ccccc54)c3)nc(-c3ccc4c(c3)oc3cccnc34)n2)cc(-n2c3ccccc3c3ccccc32)c1]

im-4-6 (15 g, 32.7 mmol) and im-a-9 (12.7 g, 34.3 mmol) were added to 300 ml of THF, and the mixture was stirred and refluxed. Then, potassium carbonate (13.6 g, 98.1 mmol) was dissolved in 100 ml of water and added thereto, and the mixture was sufficiently stirred and bis(tri-tert-butylphosphine)palladium(0) (0.2 g, 0.3 mmol) was added. After the reaction for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.5 g of Compound 56. (Yield: 62%, MS: [M+H]+=666)

### Synthesis Example 57

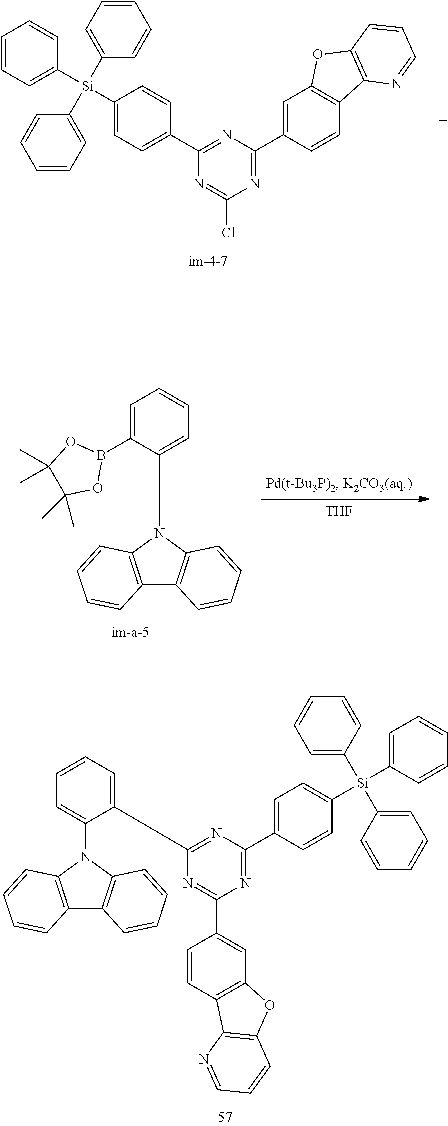 [SMILES: Clc1nc(-c2ccc([Si](c3ccccc3)(c3ccccc3)c3ccccc3)cc2)nc(-c2ccc3c(c2)oc2cccnc23)n1 ; CC1(C)OB(c2ccccc2-n2c3ccccc3c3ccccc32)OC1(C)C ; "Pd(t-Bu3P)2, K2CO3(aq.)" ; "THF" ; c1ccc([Si](c2ccccc2)(c2ccccc2)c2ccc(-c3nc(-c4ccc5c(c4)oc4cccnc45)nc(-c4ccccc4-n4c5ccccc5c5ccccc54)n3)cc2)cc1]

im-4-7 (15 g, 24.3 mmol) and im-a-5 (9.4 g, 25.5 mmol) were added to 300 ml of THF, and the mixture was stirred and refluxed. Then, potassium carbonate (10.1 g, 72.9 mmol) was dissolved in 100 ml of water and added thereto, and the mixture was sufficiently stirred and bis(tri-tert-butylphosphine)palladium(0) (0.1 g, 0.2 mmol) was added. After the reaction for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13 g of Compound 57. (Yield: 65%, MS: [M+H]+=824)

### Synthesis Example 58

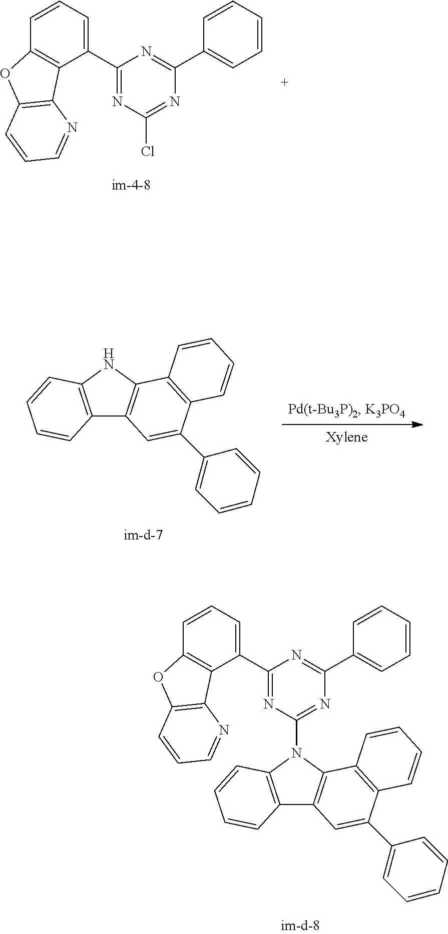

im-4-8 (15 g, 41.8 mmol), im-d-7 (12.9 g, 43.9 mmol) and potassium phosphate (26.6 g, 125.4 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 18.5 g of Compound 58. (Yield: 72%, MS: [M+H]+=616)

### Synthesis Example 59

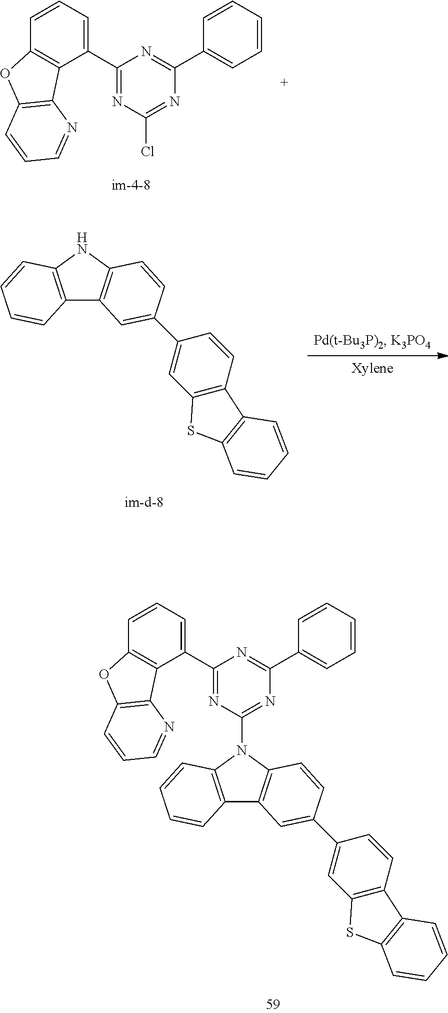

im-4-8 (15 g, 41.8 mmol), im-d-8 (15.3 g, 43.9 mmol) and potassium phosphate (26.6 g, 125.4 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 4 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 18.5 g of Compound 59. (Yield: 66%, MS: [M+H]+=672)

### Synthesis Example 60

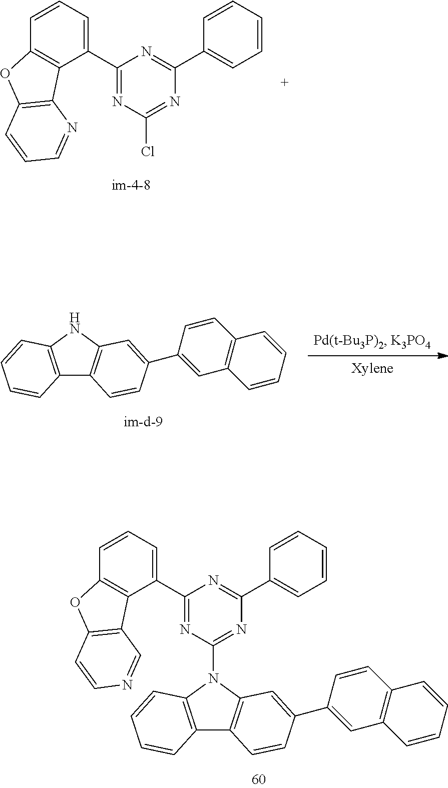

im-4-8 (15 g, 41.8 mmol), im-d-9 (12.9 g, 43.9 mmol) and potassium phosphate (26.6 g, 125.4 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 15.4 g of Compound 60. (Yield: 60%, MS: [M+H]+=616)

### Synthesis Example 61

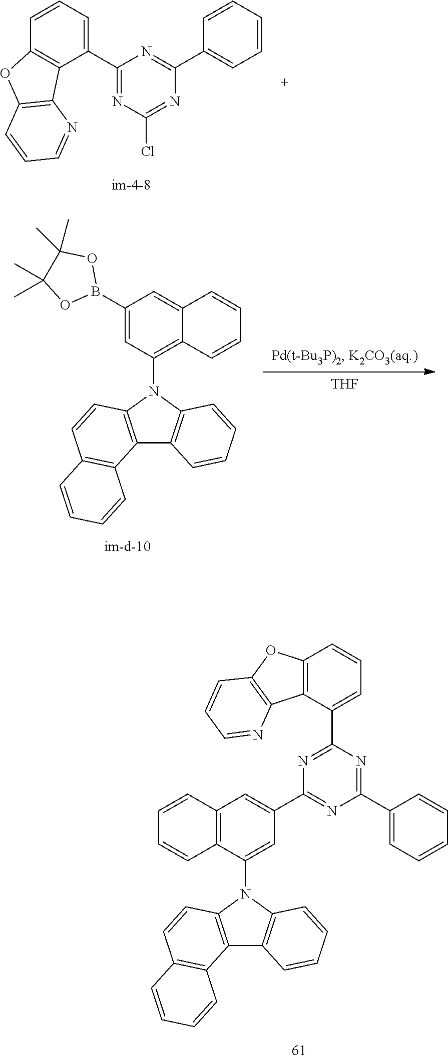 [SMILES: Clc1nc(-c2ccccc2)nc(-c2cccc3oc4cccnc4c23)n1 ; CC1(C)OB(c2cc(-n3c4ccccc4c4c5ccccc5ccc43)c3ccccc3c2)OC1(C)C ; "Pd(t-Bu3P)2, K2CO3(aq.)" ; "THF" ; c1ccc(-c2nc(-c3cc(-n4c5ccccc5c5c6ccccc6ccc54)c4ccccc4c3)nc(-c3cccc4oc5cccnc5c34)n2)cc1]

im-4-8 (15 g, 41.8 mmol) and im-d-10 (20.6 g, 43.9 mmol) were added to 300 ml of THE, and the mixture was stirred and refluxed. Then, potassium carbonate (17.3 g, 125.4 mmol) was dissolved in 100 ml of water and added thereto, and the mixture was sufficiently stirred and bis(tri-tert-butylphosphine)palladium(0) (0.2 g, 0.4 mmol) was added. After the reaction for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.7 g of Compound 61. (Yield: 60%, MS: [M+H]+=666)

### Synthesis Example 62

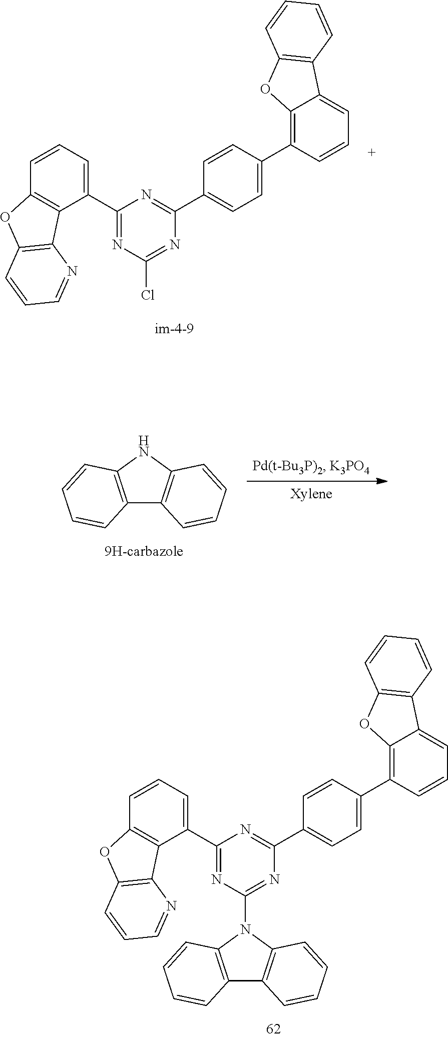

im-4-9 (15 g, 28.6 mmol), 9H-carbazole (5 g, 30 mmol) and potassium phosphate (18.2 g, 85.7 mmol) were added to 300 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13.7 g of Compound 62. (Yield: 73%, MS: [M+H]+=656)

### Synthesis Example 63

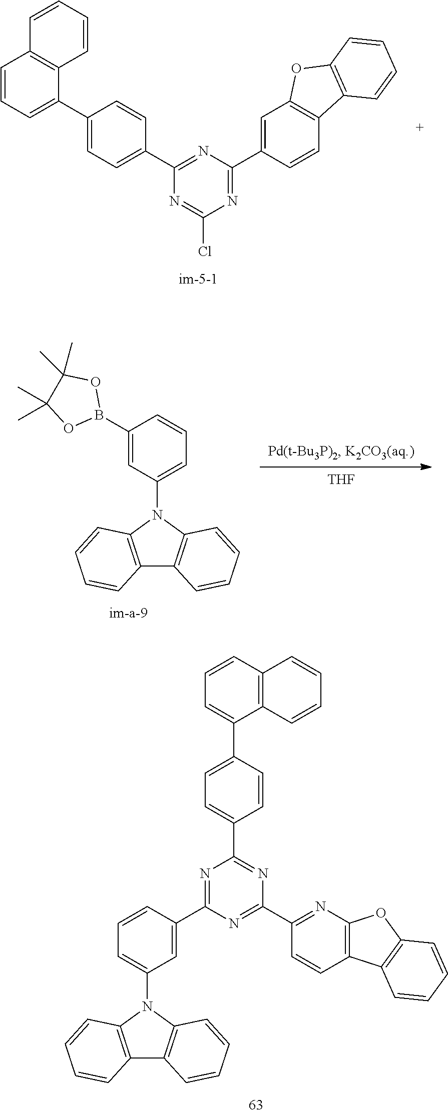 [SMILES: Clc1nc(-c2ccc(-c3cccc4ccccc34)cc2)nc(-c2ccc3c(c2)oc2ccccc23)n1 ; CC1(C)OB(c2cccc(-n3c4ccccc4c4ccccc43)c2)OC1(C)C ; "Pd(t-Bu3P)2, K2CO3(aq.)" ; "THF" ; c1cc(-c2nc(-c3ccc(-c4cccc5ccccc45)cc3)nc(-c3ccc4c(n3)oc3ccccc34)n2)cc(-n2c3ccccc3c3ccccc32)c1]

im-5-1 (15 g, 30.9 mmol) and im-a-9 (12 g, 32.5 mmol) were added to 300 ml of THF, and the mixture was stirred and refluxed. Then, potassium carbonate (12.8 g, 92.8 mmol) was dissolved in 100 ml of water and added thereto, and the mixture was sufficiently stirred and bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.9 g of Compound 63. (Yield: 65%, MS: [M+H]+=692)

### Synthesis Example 64

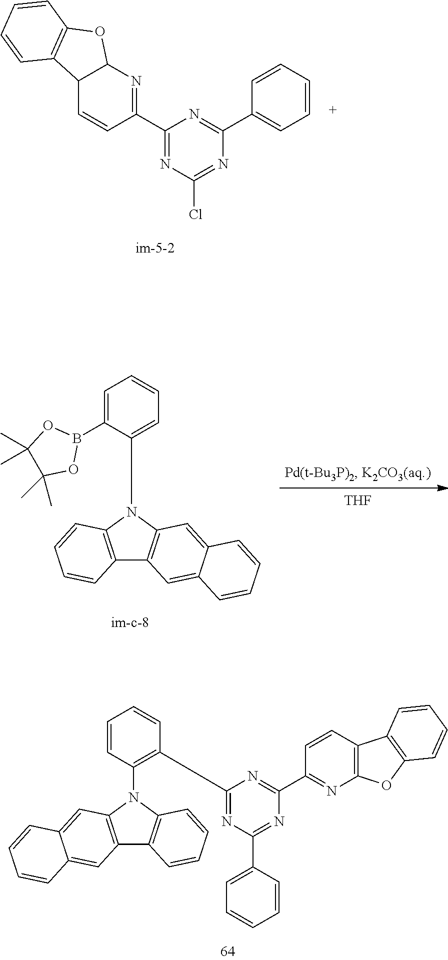 [SMILES: Clc1nc(C2=NC3Oc4ccccc4C3C=C2)nc(-c2ccccc2)n1 ; CC1(C)OB(c2ccccc2-n2c3ccccc3c3cc4ccccc4cc32)OC1(C)C ; "Pd(t-Bu3P)2, K2CO3(aq.)" ; "THF" ; c1ccc(-c2nc(-c3ccc4c(n3)oc3ccccc34)nc(-c3ccccc3-n3c4ccccc4c4cc5ccccc5cc43)n2)cc1]

im-5-2 (15 g, 41.6 mmol) and im-c-8 (18.3 g, 43.7 mmol) were added to 300 ml of THE, and the mixture was stirred and refluxed. Then, potassium carbonate (17.2 g, 124.7 mmol) was dissolved in 100 ml of water and added thereto, and the mixture was sufficiently stirred and bis(tri-tert-butylphosphine)palladium(0) (0.2 g, 0.4 mmol) was added. After the reaction for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 18.9 g of Compound 64. (Yield: 74%, MS: [M+H]+=616)

### Synthesis Example 65

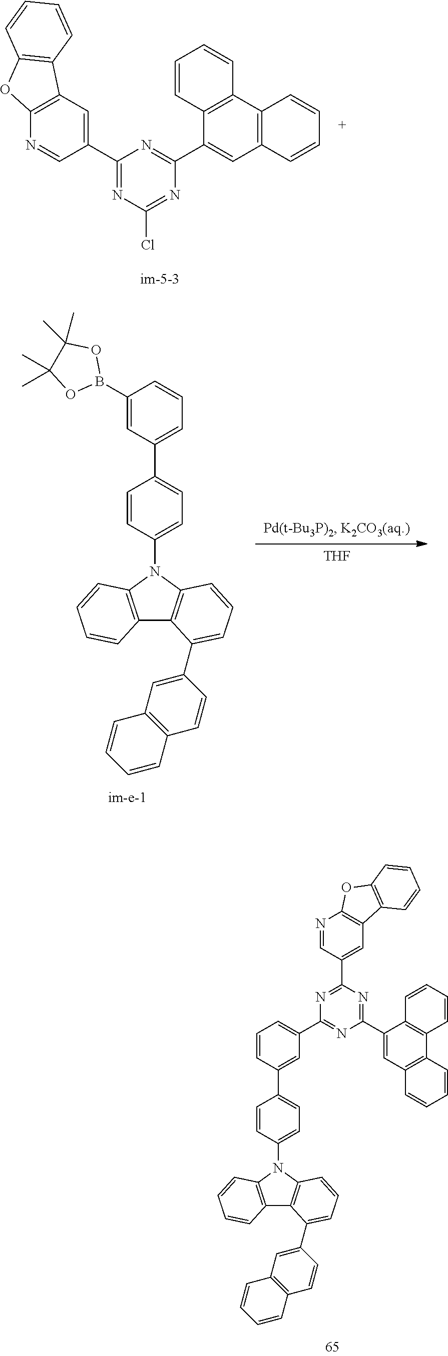 [SMILES: Clc1nc(-c2cnc3oc4ccccc4c3c2)nc(-c2cc3ccccc3c3ccccc23)n1 ; CC1(C)OB(c2cccc(-c3ccc(-n4c5ccccc5c5c(-c6ccc7ccccc7c6)cccc54)cc3)c2)OC1(C)C ; "Pd(t-Bu3P)2, K2CO3(aq.)" ; "THF" ; c1cc(-c2ccc(-n3c4ccccc4c4c(-c5ccc6ccccc6c5)cccc43)cc2)cc(-c2nc(-c3cnc4oc5ccccc5c4c3)nc(-c3cc4ccccc4c4ccccc34)n2)c1]

im-5-3 (15 g, 32.7 mmol) and im-e-1 (19.6 g, 34.3 mmol) were added to 300 ml of THF, and the mixture was stirred and refluxed. Then, potassium carbonate (13.6 g, 98.1 mmol) was dissolved in 100 ml of water and added thereto, and the mixture was sufficiently stirred and bis(tri-tert-butylphosphine)palladium(0) (0.2 g, 0.3 mmol) was added. After the reaction for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 21 g of Compound 65. (Yield: 74%, MS: [M+H]+=868)

### Synthesis Example 66

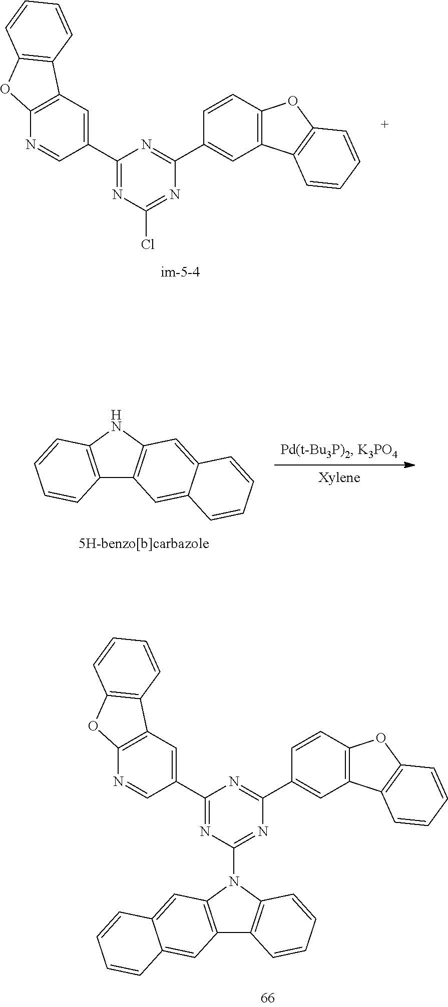

im-5-4 (15 g, 33.4 mmol), 5H-benzo[b]carbazole (7.6 g, 35.1 mmol) and potassium phosphate (21.3 g, 100.3 mmol) were added to 300 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 4 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13 g of Compound 66. (Yield: 62%, MS: [M+H]+=630)

### Synthesis Example 67

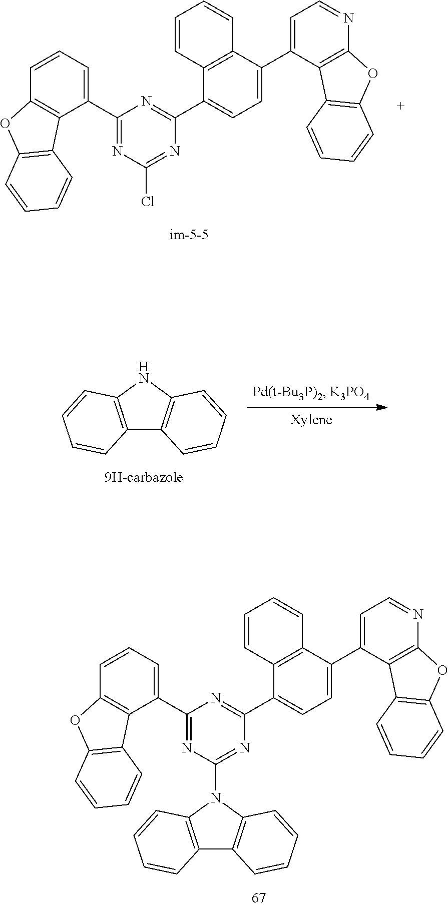

im-5-5 (15 g, 26.1 mmol), 9H-carbazole (4.6 g, 27.4 mmol) and potassium phosphate (16.6 g, 78.3 mmol) were added to 300 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.6 g of Compound 67. (Yield: 63%, MS: [M+H]+=706)

### Synthesis Example 68

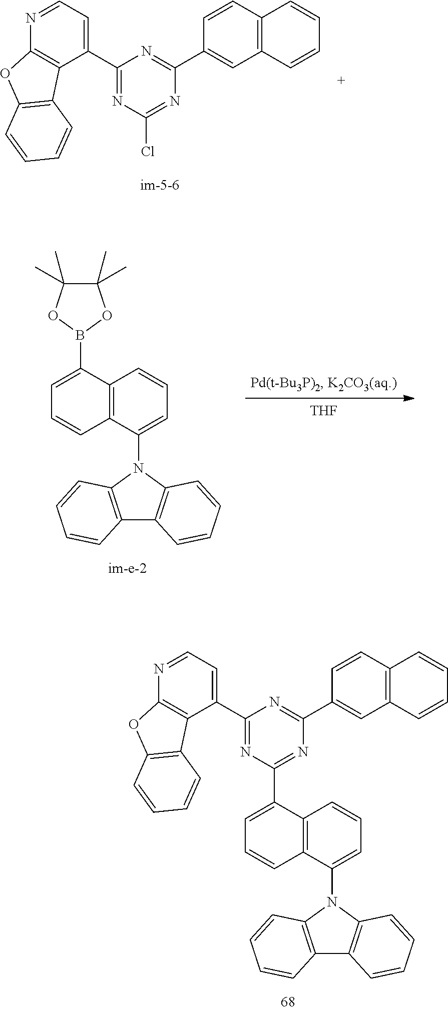 [SMILES: Clc1nc(-c2ccc3ccccc3c2)nc(-c2ccnc3oc4ccccc4c23)n1 ; CC1(C)OB(c2cccc3c(-n4c5ccccc5c5ccccc54)cccc23)OC1(C)C ; "Pd(t-Bu3P)2, K2CO3(aq.)" ; "THF" ; c1ccc2cc(-c3nc(-c4cccc5c(-n6c7ccccc7c7ccccc76)cccc45)nc(-c4ccnc5oc6ccccc6c45)n3)ccc2c1]

im-5-6 (15 g, 36.7 mmol) and im-e-2 (16.2 g, 38.5 mmol) were added to 300 ml of THF, and the mixture was stirred and refluxed. Then, potassium carbonate (15.2 g, 110.1 mmol) was dissolved in 100 ml of water and added thereto, and the mixture was sufficiently stirred and bis(tri-tert-butylphosphine)palladium(0) (0.2 g, 0.4 mmol) was added. After the reaction for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.6 g of Compound 68. (Yield: 68%, MS: [M+H]+=666)

### Synthesis Example 69

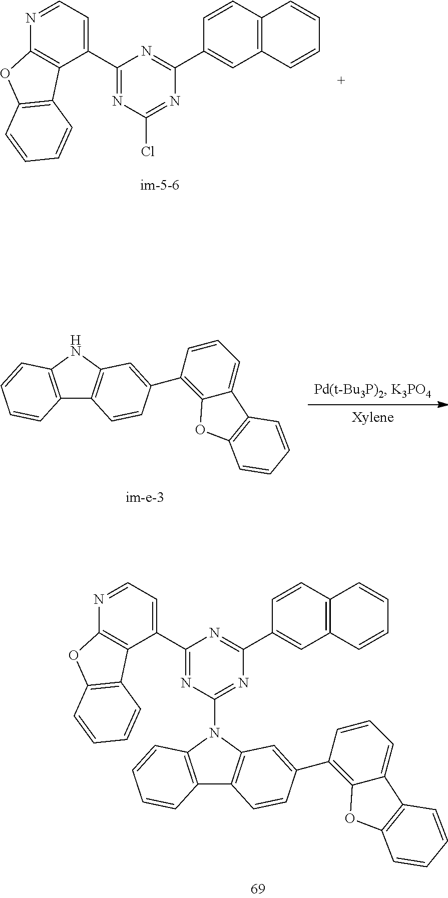

im-5-6 (15 g, 36.7 mmol), im-e-3 (12.8 g, 38.5 mmol) and potassium phosphate (23.4 g, 110.1 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 17.9 g of Compound 69. (Yield: 69%, MS: [M+H]+=706)

### Synthesis Example 70

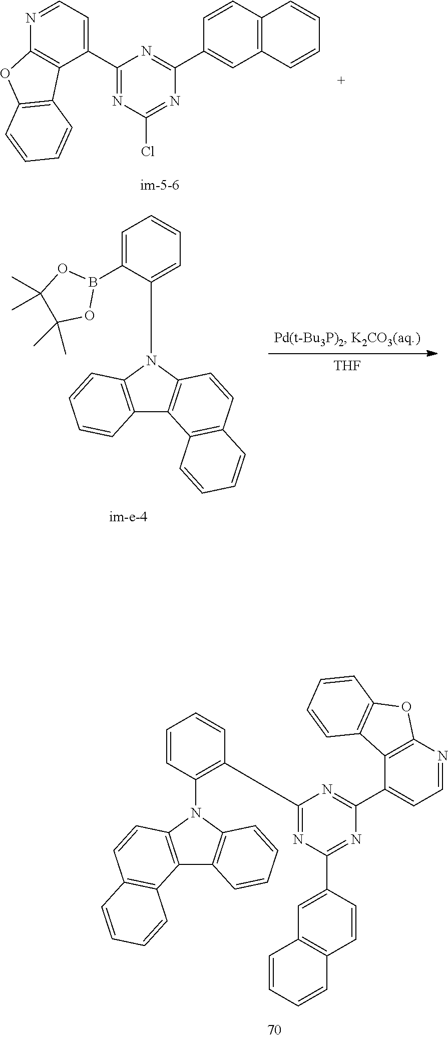 [SMILES: Clc1nc(-c2ccc3ccccc3c2)nc(-c2ccnc3oc4ccccc4c23)n1 ; CC1(C)OB(c2ccccc2-n2c3ccccc3c3c4ccccc4ccc32)OC1(C)C ; "Pd(t-Bu3P)2, K2CO3(aq.)" ; "THF" ; c1ccc(-n2c3ccccc3c3c4ccccc4ccc32)c(-c2nc(-c3ccc4ccccc4c3)nc(-c3ccnc4oc5ccccc5c34)n2)c1]

im-5-6 (15 g, 36.7 mmol) and im-e-4 (16.2 g, 38.5 mmol) were added to 300 ml of THF, and the mixture was stirred and refluxed. Then, potassium carbonate (15.2 g, 110.1 mmol) was dissolved in 100 ml of water and added thereto, and the mixture was sufficiently stirred and bis(tri-tert-butylphosphine)palladium(0) (0.2 g, 0.4 mmol) was added. After the reaction for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 18.1 g of Compound 70. (Yield: 74%, MS: [M+H]+=666)

### Synthesis Example 71

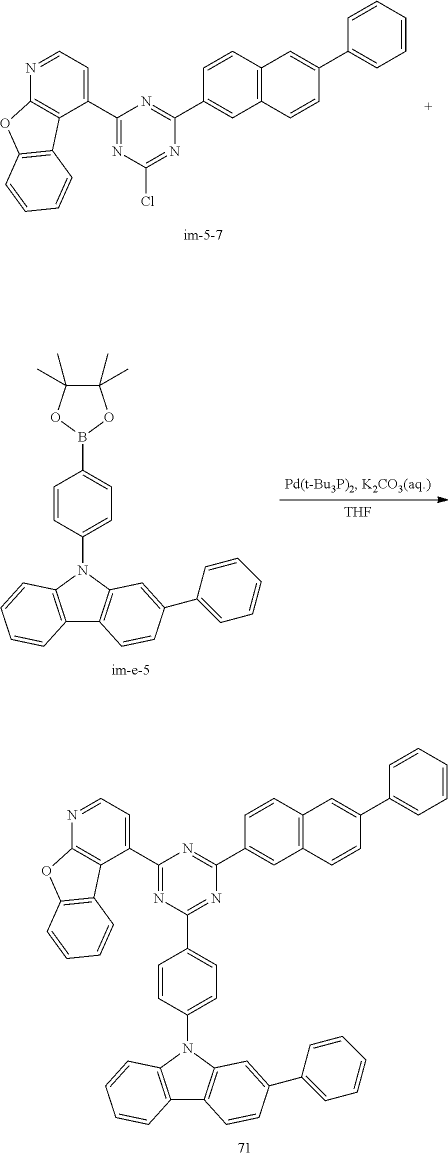 [SMILES: Clc1nc(-c2ccc3cc(-c4ccccc4)ccc3c2)nc(-c2ccnc3oc4ccccc4c23)n1 ; CC1(C)OB(c2ccc(-n3c4ccccc4c4ccc(-c5ccccc5)cc43)cc2)OC1(C)C ; "Pd(t-Bu3P)2, K2CO3(aq.)" ; "THF" ; c1ccc(-c2ccc3cc(-c4nc(-c5ccc(-n6c7ccccc7c7ccc(-c8ccccc8)cc76)cc5)nc(-c5ccnc6oc7ccccc7c56)n4)ccc3c2)cc1]

im-5-7 (15 g, 30.9 mmol) and im-e-5 (14.5 g, 32.5 mmol) were added to 300 ml of THF, and the mixture was stirred and refluxed. Then, potassium carbonate (12.8 g, 92.8 mmol) was dissolved in 100 ml of water and added thereto, and the mixture was sufficiently stirred and bis(tri-tert-butylphosphine)palladium(0) (0.2 g, 0.3 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.4 g of Compound 71. (Yield: 69%, MS: [M+H]+=768)

### Synthesis Example 72

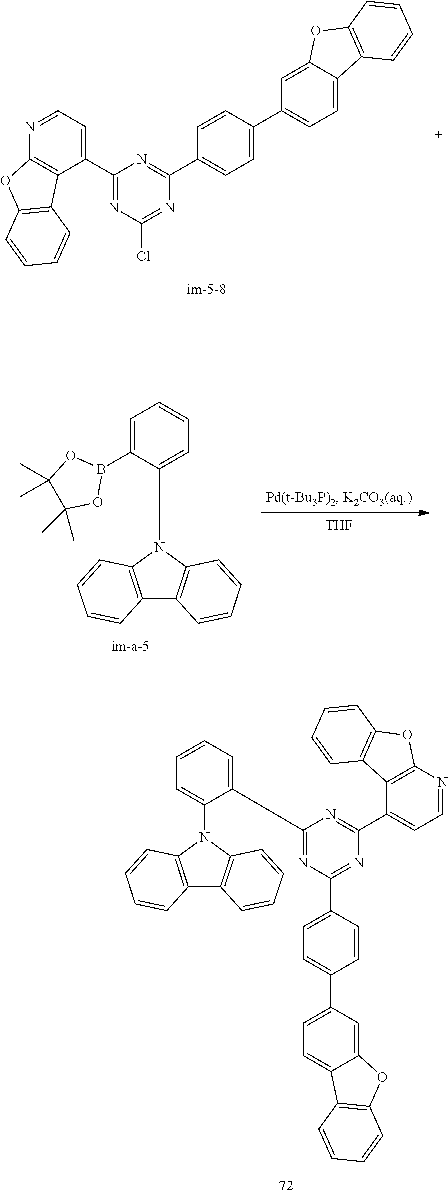 [SMILES: Clc1nc(-c2ccc(-c3ccc4c(c3)oc3ccccc34)cc2)nc(-c2ccnc3oc4ccccc4c23)n1 ; CC1(C)OB(c2ccccc2-n2c3ccccc3c3ccccc32)OC1(C)C ; "Pd(t-Bu3P)2, K2CO3(aq.)" ; "THF" ; c1ccc(-n2c3ccccc3c3ccccc32)c(-c2nc(-c3ccc(-c4ccc5c(c4)oc4ccccc45)cc3)nc(-c3ccnc4oc5ccccc5c34)n2)c1]

im-5-8 (15 g, 28.6 mmol) and im-a-5 (11.1 g, 30 mmol) were added to 300 ml of THF, and the mixture was stirred and refluxed. Then, potassium carbonate (11.8 g, 85.7 mmol) was dissolved in 100 ml of water and added thereto, and the mixture was sufficiently stirred and bis(tri-tert-butylphosphine)palladium(0) (0.1 g, 0.3 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.8 g of Compound 72. (Yield: 66%, MS: [M+H]+=732)

### Synthesis Example 73

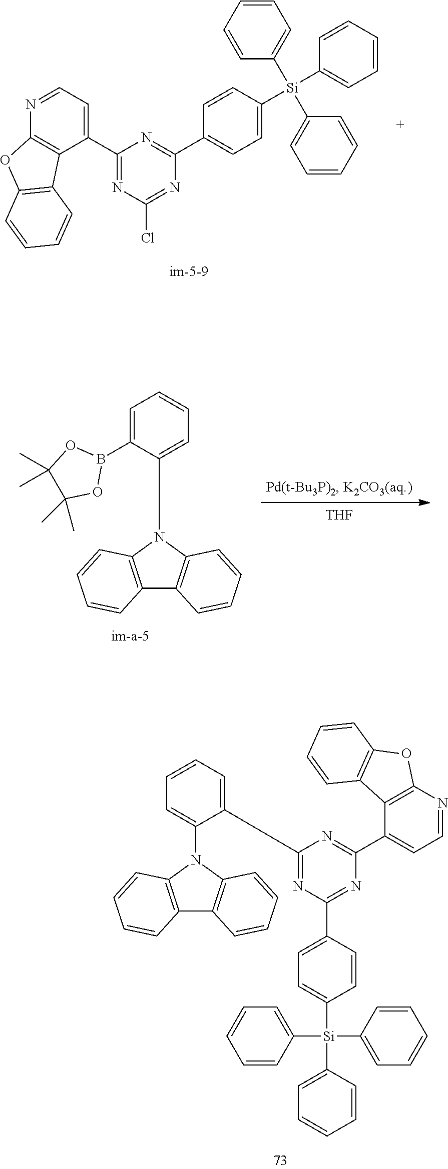 [SMILES: Clc1nc(-c2ccc([Si](c3ccccc3)(c3ccccc3)c3ccccc3)cc2)nc(-c2ccnc3oc4ccccc4c23)n1 ; CC1(C)OB(c2ccccc2-n2c3ccccc3c3ccccc32)OC1(C)C ; "Pd(t-Bu3P)2, K2CO3(aq.)" ; "THF" ; c1ccc([Si](c2ccccc2)(c2ccccc2)c2ccc(-c3nc(-c4ccccc4-n4c5ccccc5c5ccccc54)nc(-c4ccnc5oc6ccccc6c45)n3)cc2)cc1]

im-5-9 (15 g, 24.3 mmol) and im-a-5 (9.4 g, 25.5 mmol) were added to 300 ml of THF, and the mixture was stirred and refluxed. Then, potassium carbonate (10.1 g, 72.9 mmol) was dissolved in 100 ml of water and added thereto, and the mixture was sufficiently stirred and bis(tri-tert-butylphosphine)palladium(0) (0.1 g, 0.2 mmol) was added. After the reaction for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.8 g of Compound 73. (Yield: 64%, MS: [M+H]+=824)

### Synthesis Example 74

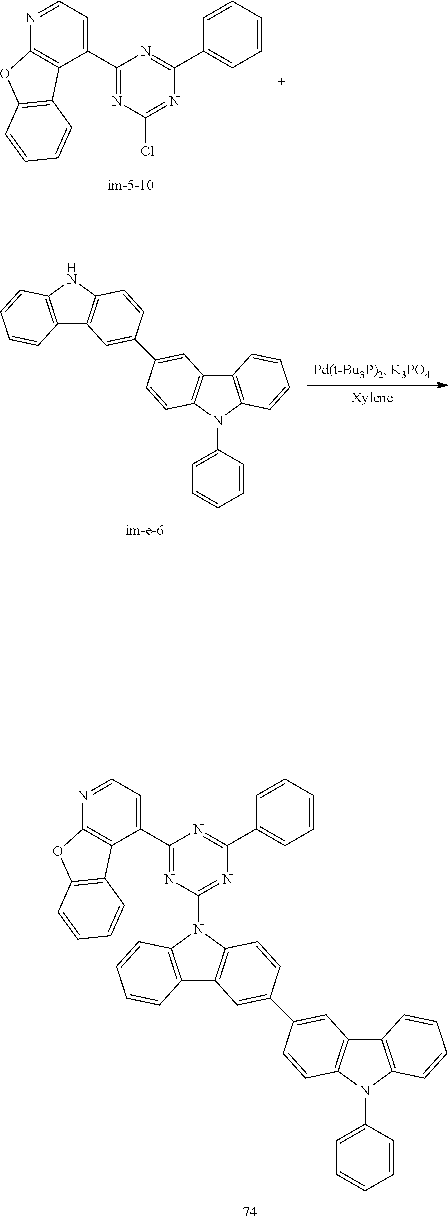

im-5-10 (15 g, 41.8 mmol), im-e-6 (17.9 g, 43.9 mmol) and potassium phosphate (26.6 g, 125.4 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 4 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 22.9 g of Compound 74. (Yield: 75%, MS: [M+H]+=731)

### Synthesis Example 75

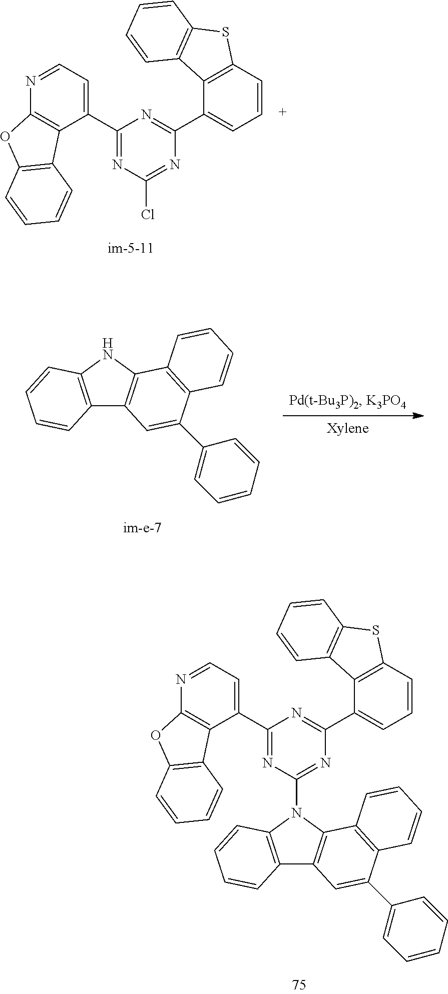

im-5-11 (15 g, 32.3 mmol), im-e-7 (9.9 g, 33.9 mmol) and potassium phosphate (20.5 g, 96.8 mmol) were added to 300 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 4 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 15.6 g of Compound 75. (Yield: 67%, MS: [M+H]+=722)

### Synthesis Example 76

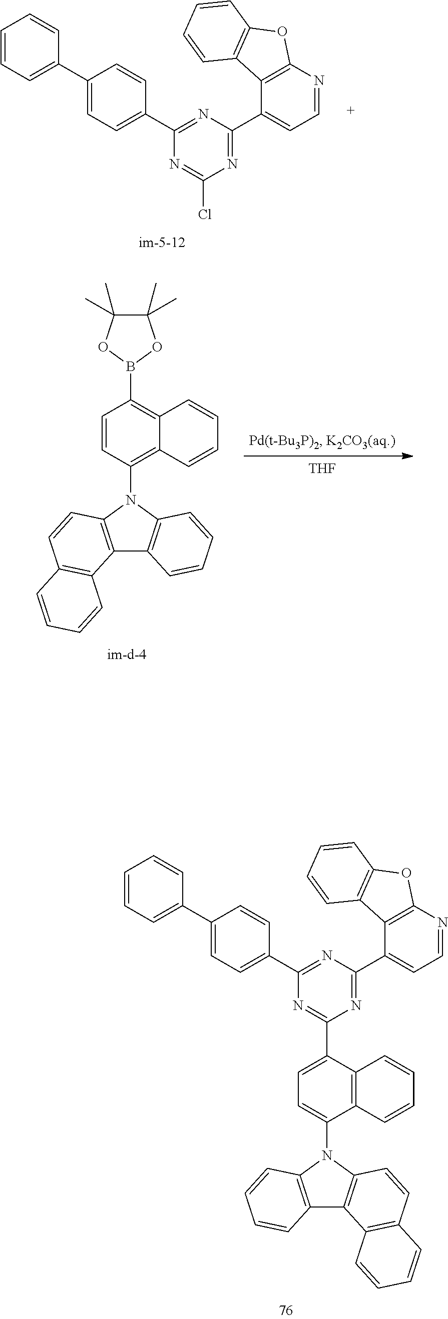 [SMILES: Clc1nc(-c2ccc(-c3ccccc3)cc2)nc(-c2ccnc3oc4ccccc4c23)n1 ; CC1(C)OB(c2ccc(-n3c4ccccc4c4c5ccccc5ccc43)c3ccccc23)OC1(C)C ; "Pd(t-Bu3P)2, K2CO3(aq.)" ; "THF" ; c1ccc(-c2ccc(-c3nc(-c4ccc(-n5c6ccccc6c6c7ccccc7ccc65)c5ccccc45)nc(-c4ccnc5oc6ccccc6c45)n3)cc2)cc1]

im-5-12 (15 g, 34.5 mmol) and im-d-4 (17 g, 36.2 mmol) were added to 300 ml of THF, and the mixture was stirred and refluxed. Then, potassium carbonate (14.3 g, 103.5 mmol) was dissolved in 100 ml of water and added thereto, and the mixture was sufficiently stirred and bis(tri-tert-butylphosphine)palladium(0) (0.2 g, 0.3 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 18.7 g of Compound 76. (Yield: 73%, MS: [M+H]+=742)

### Synthesis Example 77

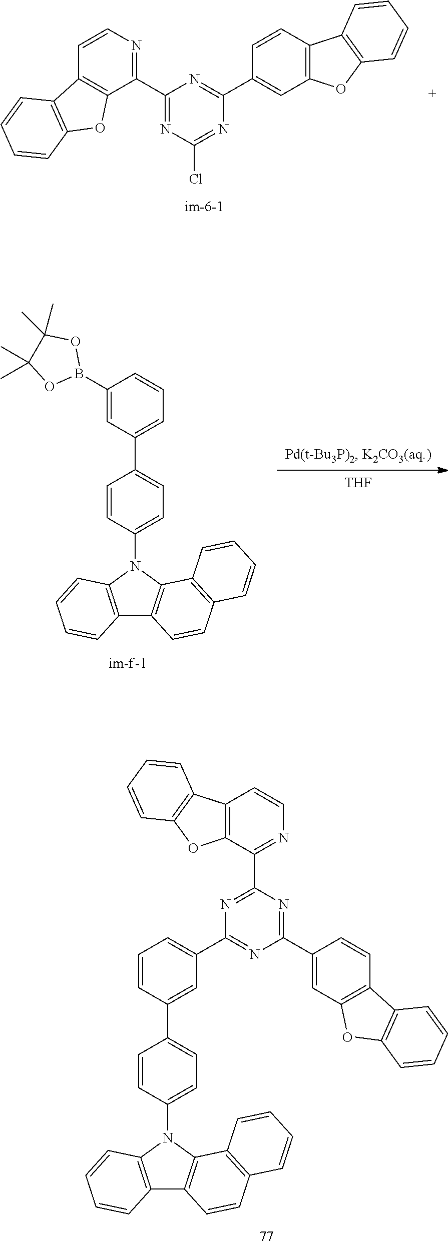 [SMILES: Clc1nc(-c2ccc3c(c2)oc2ccccc23)nc(-c2nccc3c2oc2ccccc23)n1 ; CC1(C)OB(c2cccc(-c3ccc(-n4c5ccccc5c5ccc6ccccc6c54)cc3)c2)OC1(C)C ; "Pd(t-Bu3P)2, K2CO3(aq.)" ; "THF" ; c1cc(-c2ccc(-n3c4ccccc4c4ccc5ccccc5c43)cc2)cc(-c2nc(-c3ccc4c(c3)oc3ccccc34)nc(-c3nccc4c3oc3ccccc34)n2)c1]

im-6-1 (15 g, 33.4 mmol) and im-f-1 (17.4 g, 35.1 mmol) were added to 300 ml of THF, and the mixture was stirred and refluxed. Then, potassium carbonate (13.9 g, 100.3 mmol) was dissolved in 100 ml of water and added thereto, and the mixture was sufficiently stirred and bis(tri-tert-butylphosphine)palladium(0) (0.2 g, 0.3 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 17.5 g of Compound 77. (Yield: 67%, MS: [M+H]+=782)

### Synthesis Example 78

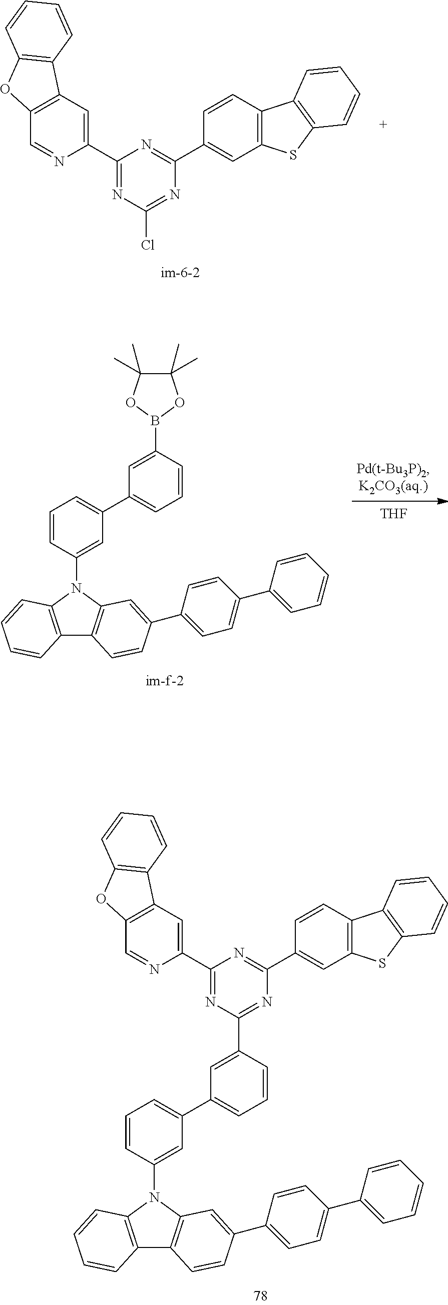 [SMILES: Clc1nc(-c2ccc3c(c2)sc2ccccc23)nc(-c2cc3c(cn2)oc2ccccc23)n1 ; CC1(C)OB(c2cccc(-c3cccc(-n4c5ccccc5c5ccc(-c6ccc(-c7ccccc7)cc6)cc54)c3)c2)OC1(C)C ; "Pd(t-Bu3P)2,K2CO3(aq.)" ; "THF" ; c1ccc(-c2ccc(-c3ccc4c5ccccc5n(-c5cccc(-c6cccc(-c7nc(-c8ccc9c(c8)sc8ccccc89)nc(-c8cc9c(cn8)oc8ccccc89)n7)c6)c5)c4c3)cc2)cc1]

im-6-2 (15 g, 32.3 mmol) and im-f-2 (20.2 g, 33.9 mmol) were added to 300 ml of THF, and the mixture was stirred and refluxed. Then, potassium carbonate (13.4 g, 96.8 mmol) was dissolved in 100 ml of water and added thereto, and the mixture was sufficiently stirred and bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 18.6 g of Compound 78. (Yield: 64%, MS: [M+H]+=900)

### Synthesis Example 79

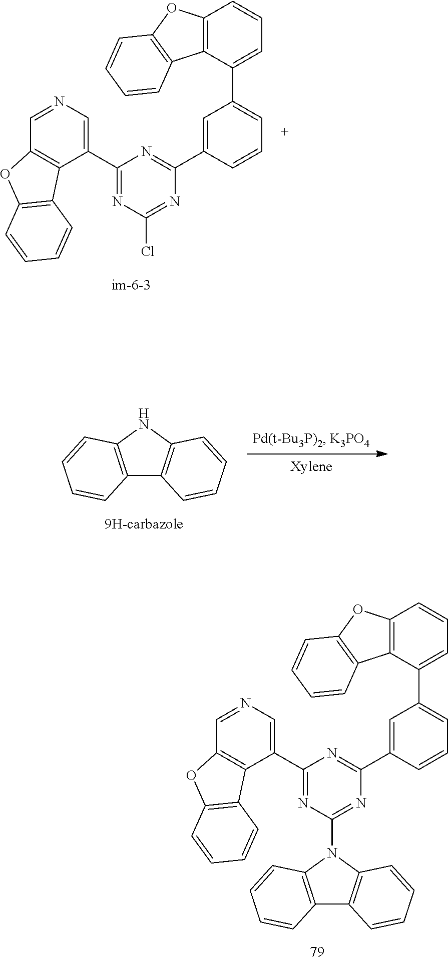

im-6-3 (15 g, 28.6 mmol), 9H-carbazole (5 g, 30 mmol) and potassium phosphate (18.2 g, 85.7 mmol) were added to 300 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.9 g of Compound 79. (Yield: 69%, MS: [M+H]+=656)

### Synthesis Example 80

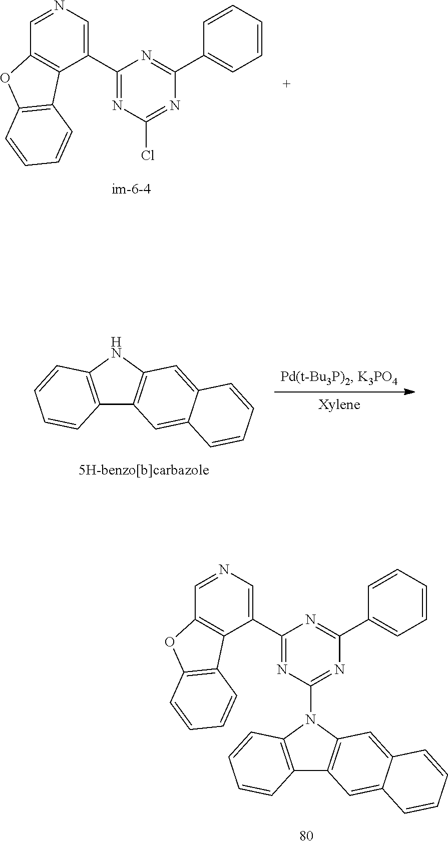

im-6-4 (15 g, 41.8 mmol), 5H-benzo[b]carbazole (9.5 g, 43.9 mmol) and potassium phosphate (26.6 g, 125.4 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 4 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 15.1 g of Compound 80. (Yield: 67%, MS: [M+H]+=540)

### Synthesis Example 81

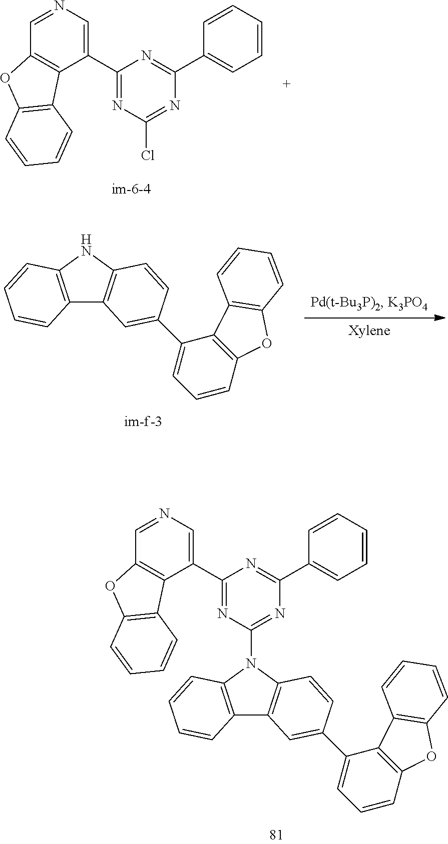

im-6-4 (15 g, 41.8 mmol), im-f-3 (14.6 g, 43.9 mmol) and potassium phosphate (26.6 g, 125.4 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 19.4 g of Compound 81. (Yield: 71%, MS: [M+H]+=656)

### Synthesis Example 82

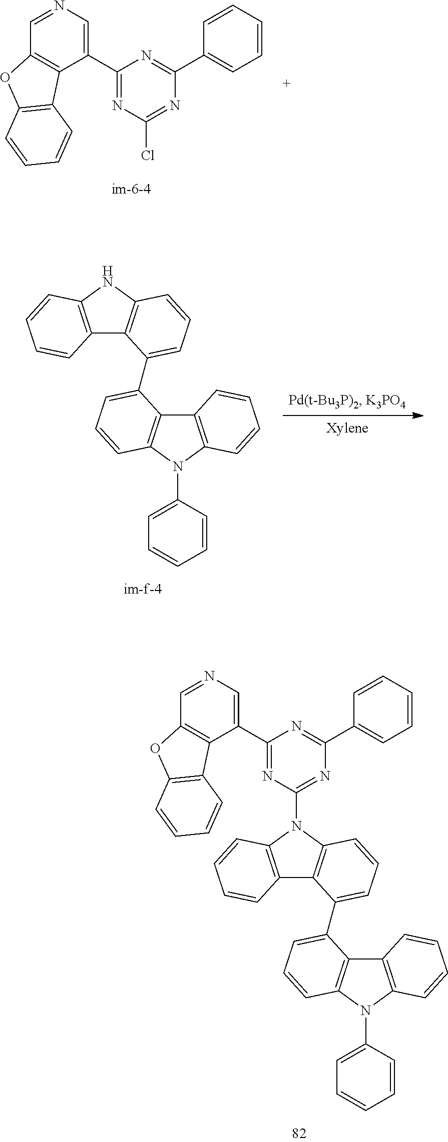

im-6-4 (15 g, 41.8 mmol), im-f-4 (17.9 g, 43.9 mmol) and potassium phosphate (26.6 g, 125.4 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 22.9 g of Compound 82. (Yield: 75%, MS: [M+H]+=731)

### Synthesis Example 83

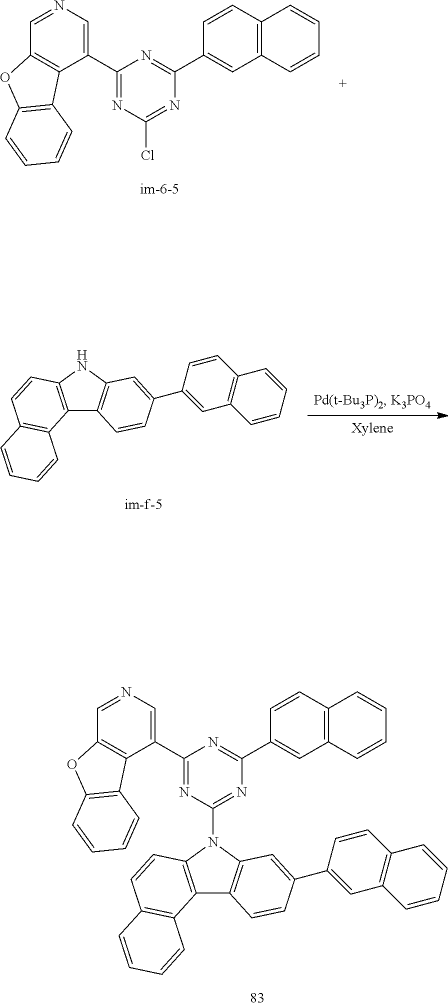

im-6-5 (15 g, 36.7 mmol), im-f-5 (13.2 g, 38.5 mmol) and potassium phosphate (23.4 g, 110.1 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 18.1 g of Compound 83. (Yield: 69%, MS: [M+H]+=716)

### Synthesis Example 84

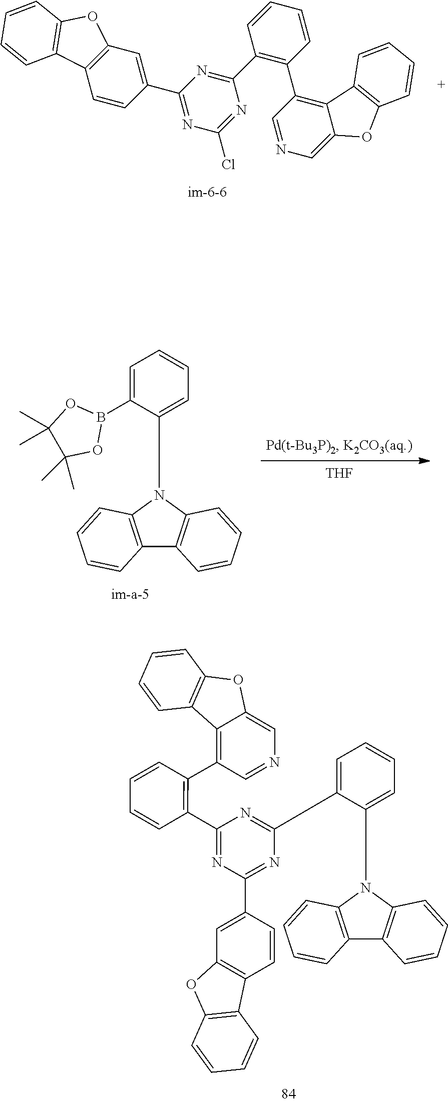 [SMILES: Clc1nc(-c2ccc3c(c2)oc2ccccc23)nc(-c2ccccc2-c2cncc3oc4ccccc4c23)n1 ; CC1(C)OB(c2ccccc2-n2c3ccccc3c3ccccc32)OC1(C)C ; "Pd(t-Bu3P)2, K2CO3(aq.)" ; "THF" ; c1ccc(-c2cncc3oc4ccccc4c23)c(-c2nc(-c3ccc4c(c3)oc3ccccc34)nc(-c3ccccc3-n3c4ccccc4c4ccccc43)n2)c1]

im-6-6 (15 g, 28.6 mmol) and im-a-5 (11.1 g, 30 mmol) were added to 300 ml of THF, and the mixture was stirred and refluxed. Then, potassium carbonate (11.8 g, 85.7 mmol) was dissolved in 100 ml of water and added thereto, and the mixture was sufficiently stirred and bis(tri-tert-butylphosphine)palladium(0) (0.1 g, 0.3 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.7 g of Compound 84. (Yield: 61%, MS: [M+H]+=732)

### Synthesis Example 85

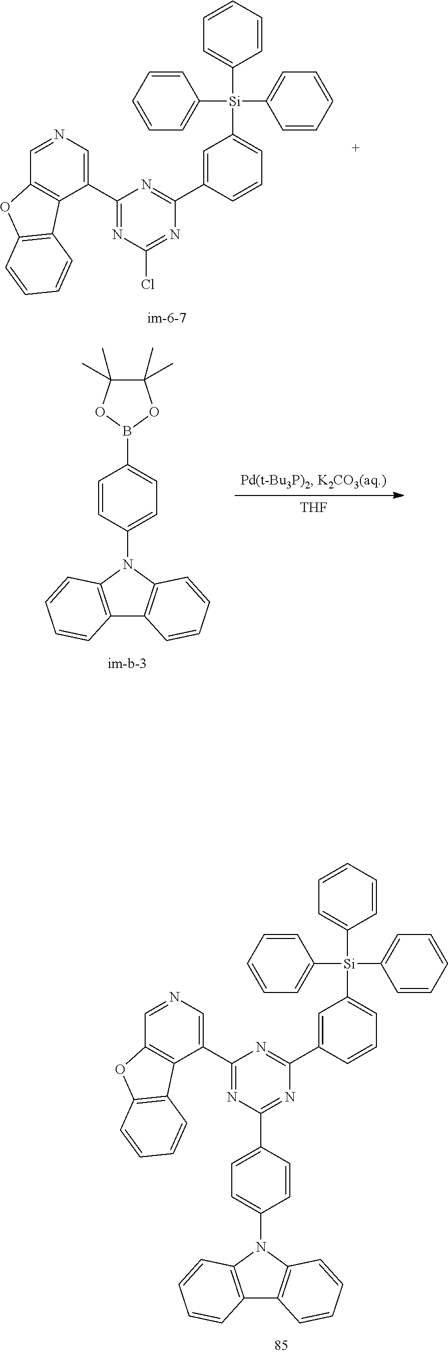 [SMILES: Clc1nc(-c2cccc([Si](c3ccccc3)(c3ccccc3)c3ccccc3)c2)nc(-c2cncc3oc4ccccc4c23)n1 ; CC1(C)OB(c2ccc(-n3c4ccccc4c4ccccc43)cc2)OC1(C)C ; "Pd(t-Bu3P)2, K2CO3(aq.)" ; "THF" ; c1ccc([Si](c2ccccc2)(c2ccccc2)c2cccc(-c3nc(-c4ccc(-n5c6ccccc6c6ccccc65)cc4)nc(-c4cncc5oc6ccccc6c45)n3)c2)cc1]

im-6-7 (15 g, 24.3 mmol) and im-b-3 (9.4 g, 25.5 mmol) were added to 300 ml of THF, and the mixture was stirred and refluxed. Then, potassium carbonate (10.1 g, 72.9 mmol) was dissolved in 100 ml of water and added thereto, and the mixture was sufficiently stirred and bis(tri-tert-butylphosphine)palladium(0) (0.1 g, 0.2 mmol) was added. After the reaction for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.8 g of Compound 84. (Yield: 74%, MS: [M+H]+=824)

### Synthesis Example 86

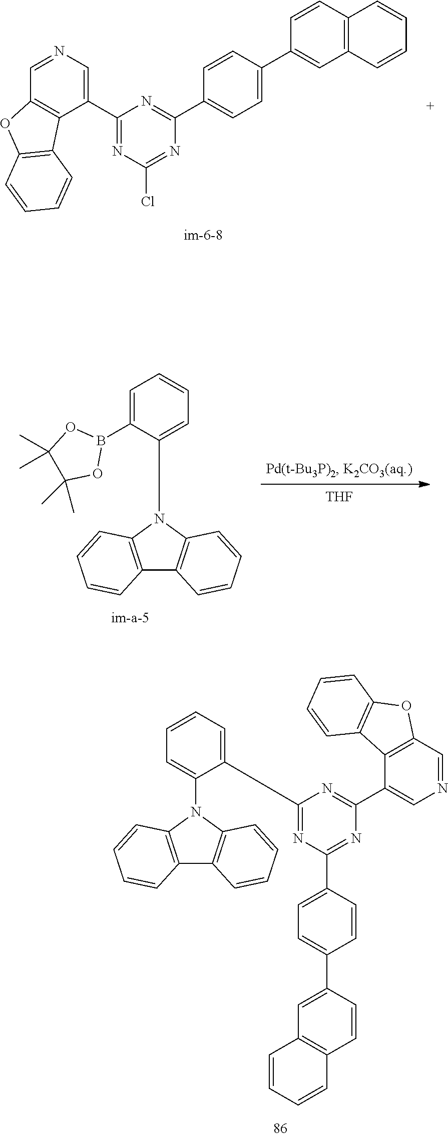 [SMILES: Clc1nc(-c2ccc(-c3ccc4ccccc4c3)cc2)nc(-c2cncc3oc4ccccc4c23)n1 ; CC1(C)OB(c2ccccc2-n2c3ccccc3c3ccccc32)OC1(C)C ; "Pd(t-Bu3P)2, K2CO3(aq.)" ; "THF" ; c1ccc(-n2c3ccccc3c3ccccc32)c(-c2nc(-c3ccc(-c4ccc5ccccc5c4)cc3)nc(-c3cncc4oc5ccccc5c34)n2)c1]

im-6-8 (15 g, 30.9 mmol) and im-a-5 (12 g, 32.5 mmol) were added to 300 ml of THF, and the mixture was stirred and refluxed. Then, potassium carbonate (12.8 g, 92.8 mmol) was dissolved in 100 ml of water and added thereto, and the mixture was sufficiently stirred and bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.8 g of Compound 86. (Yield: 69%, MS: [M+H]+=692)

### Synthesis Example 87

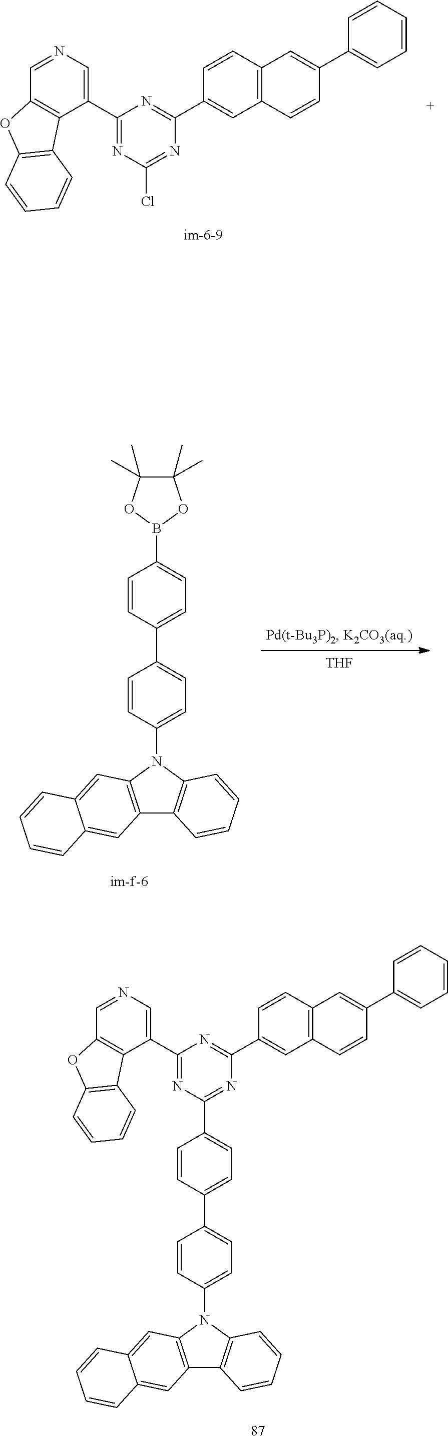 [SMILES: Clc1nc(-c2ccc3cc(-c4ccccc4)ccc3c2)nc(-c2cncc3oc4ccccc4c23)n1 ; CC1(C)OB(c2ccc(-c3ccc(-n4c5ccccc5c5cc6ccccc6cc54)cc3)cc2)OC1(C)C ; "Pd(t-Bu3P)2, K2CO3(aq.)" ; "THF" ; c1ccc(-c2ccc3cc(-c4nc(-c5ccc(-c6ccc(-n7c8ccccc8c8cc9ccccc9cc87)cc6)cc5)nc(-c5cncc6oc7ccccc7c56)n4)ccc3c2)cc1]

im-6-9 (15 g, 30.9 mmol) and im-f-6 (16.1 g, 32.5 mmol) were added to 300 ml of THF, and the mixture was stirred and refluxed. Then, potassium carbonate (12.8 g, 92.8 mmol) was dissolved in 100 ml of water and added thereto, and the mixture was sufficiently stirred and bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.4 g of Compound 87. (Yield: 61%, MS: [M+H]+=818)

### Synthesis Example 88

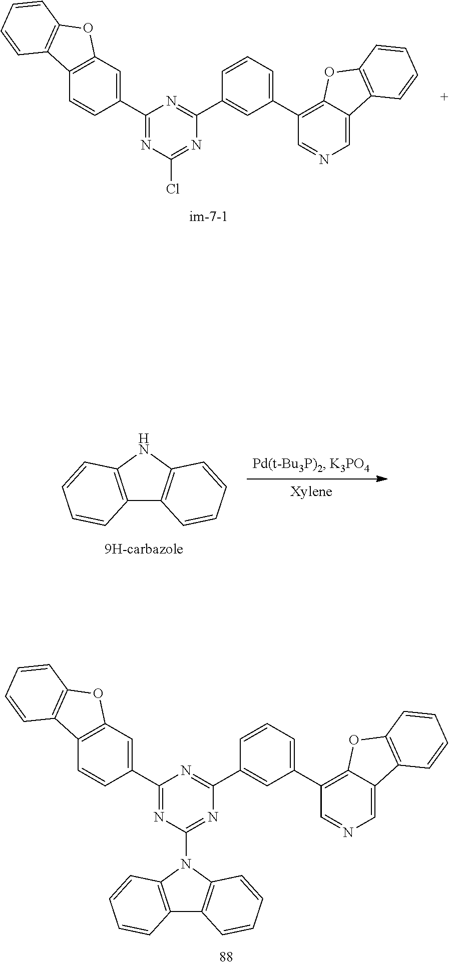

im-7-1 (15 g, 28.6 mmol), 9H-carbazole (5 g, 30 mmol) and potassium phosphate (18.2 g, 85.7 mmol) were added to 300 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13.9 g of Compound 88. (Yield: 74%, MS: [M+H]+=656)

### Synthesis Example 89

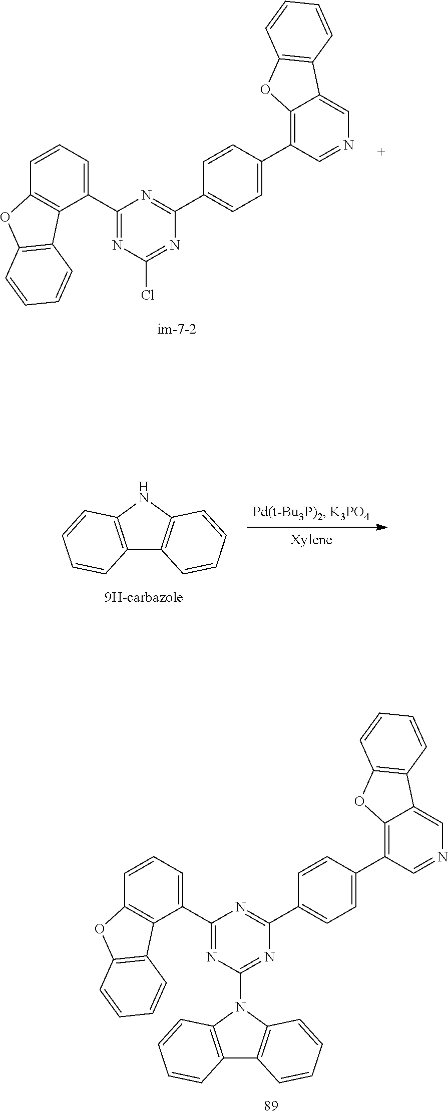

im-7-2 (15 g, 28.6 mmol), 9H-carbazole (5 g, 30 mmol) and potassium phosphate (18.2 g, 85.7 mmol) were added to 300 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13.5 g of Compound 89. (Yield: 72%, MS: [M+H]+=656)

### Synthesis Example 90

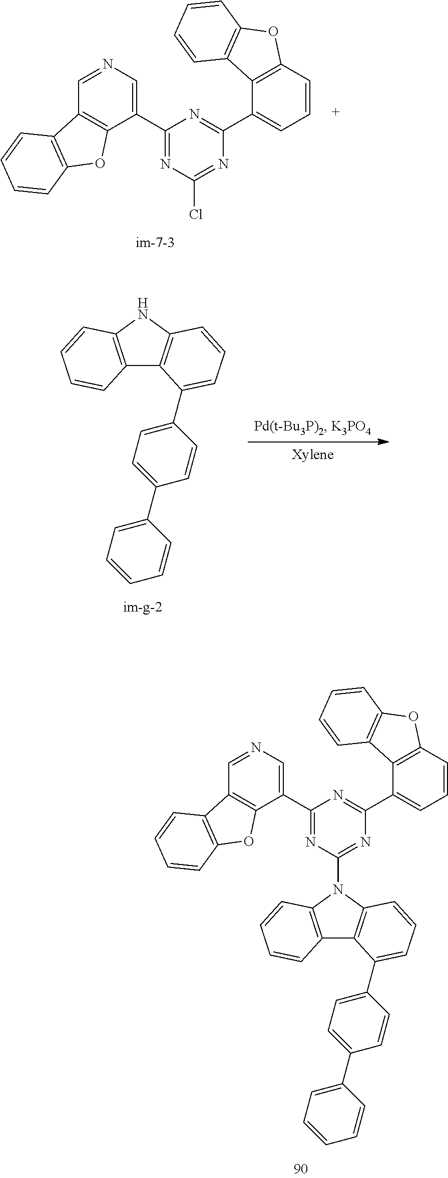

im-7-3 (15 g, 33.4 mmol), im-g-2 (11.2 g, 35.1 mmol) and potassium phosphate (21.3 g, 100.3 mmol) were added to 300 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 17.6 g of Compound 90. (Yield: 72%, MS: [M+H]+=732)

### Synthesis Example 91

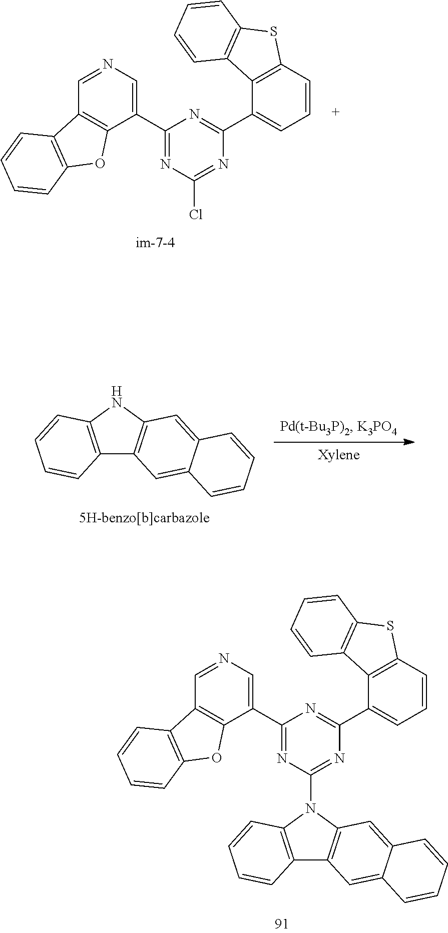

im-7-4 (15 g, 32.3 mmol), 5H-benzo[b]carbazole (7.4 g, 33.9 mmol) and potassium phosphate (20.5 g, 96.8 mmol) were added to 300 ml of xylene under a nitrogen atmosphere, and the mixture was stirred and refluxed. Then, bis(tri-tert-butylphosphine) palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.5 g of Compound 91. (Yield: 60%, MS: [M+H]+=646)

### Synthesis Example 92

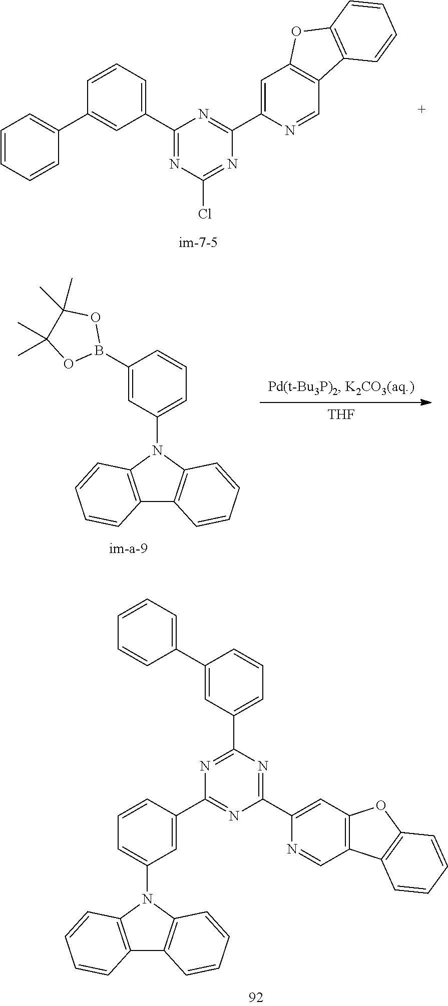 [SMILES: Clc1nc(-c2cccc(-c3ccccc3)c2)nc(-c2cc3oc4ccccc4c3cn2)n1 ; CC1(C)OB(c2cccc(-n3c4ccccc4c4ccccc43)c2)OC1(C)C ; "Pd(t-Bu3P)2, K2CO3(aq.)" ; "THF" ; c1ccc(-c2cccc(-c3nc(-c4cccc(-n5c6ccccc6c6ccccc65)c4)nc(-c4cc5oc6ccccc6c5cn4)n3)c2)cc1]

im-7-5 (15 g, 34.5 mmol) and im-a-9 (13.4 g, 36.2 mmol) were added to 300 ml of THF, and the mixture was stirred and refluxed. Then, potassium carbonate (14.3 g, 103.5 mmol) was dissolved in 100 ml of water and added thereto, and the mixture was sufficiently stirred and bis(tri-tert-butylphosphine)palladium(0) (0.2 g, 0.3 mmol) was added. After the reaction for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.2 g of Compound 92. (Yield: 64%, MS: [M+H]+=642)

### Synthesis Example 93

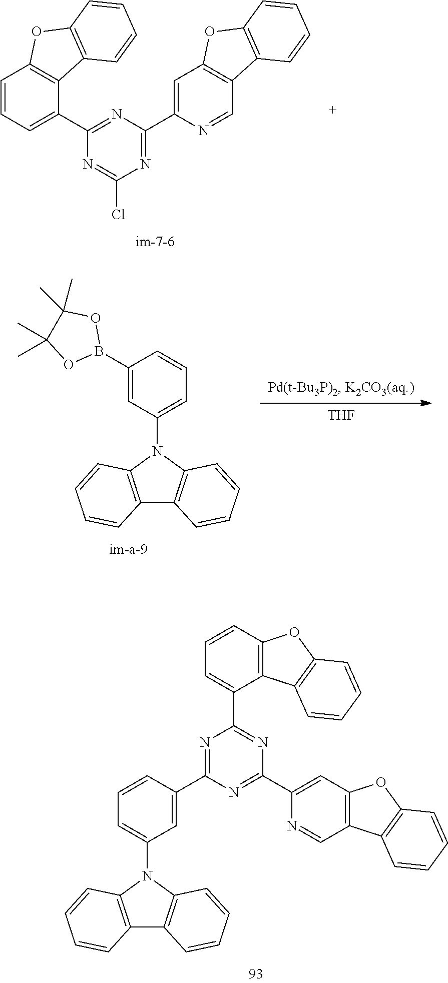 [SMILES: Clc1nc(-c2cc3oc4ccccc4c3cn2)nc(-c2cccc3oc4ccccc4c23)n1 ; CC1(C)OB(c2cccc(-n3c4ccccc4c4ccccc43)c2)OC1(C)C ; "Pd(t-Bu3P)2, K2CO3(aq.)" ; "THF" ; c1cc(-c2nc(-c3cc4oc5ccccc5c4cn3)nc(-c3cccc4oc5ccccc5c34)n2)cc(-n2c3ccccc3c3ccccc32)c1]

im-7-6 (15 g, 33.4 mmol) and im-a-9 (13 g, 35.1 mmol) were added to 300 ml of THF, and the mixture was stirred and refluxed. Then, potassium carbonate (13.9 g, 100.3 mmol) was dissolved in 100 ml of water and added thereto, and the mixture was sufficiently stirred and bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16 g of Compound 93. (Yield: 73%, MS: [M+H]+=656)

### Synthesis Example 94

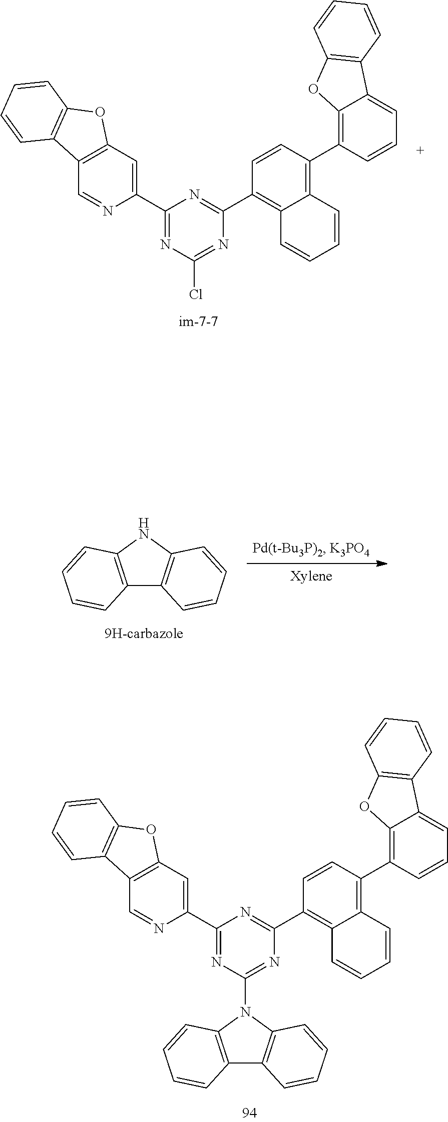

im-7-7 (15 g, 26.1 mmol), 9H-carbazole (4.6 g, 27.4 mmol) and potassium phosphate (16.6 g, 78.3 mmol) were added to 300 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13.2 g of Compound 94. (Yield: 72%, MS: [M+H]+=706)

### Synthesis Example 95

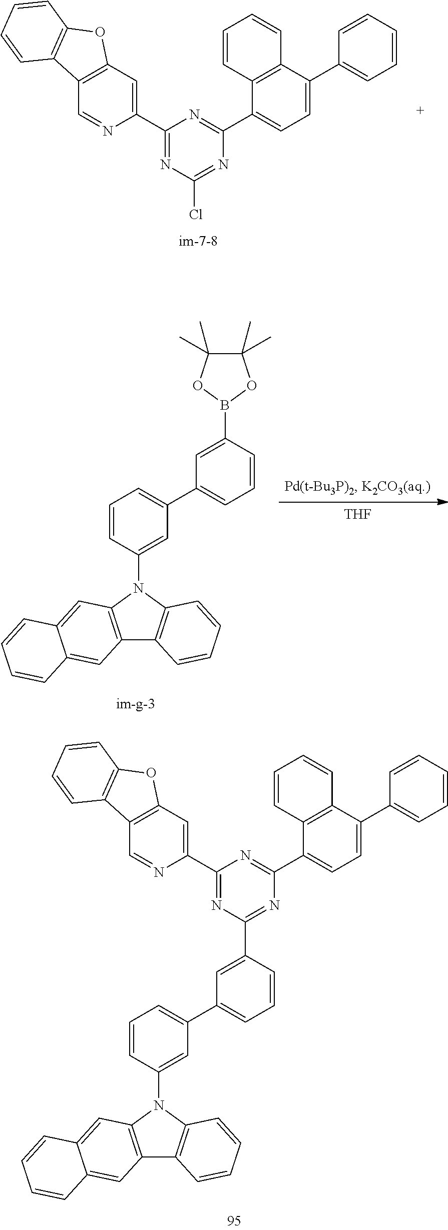 [SMILES: Clc1nc(-c2cc3oc4ccccc4c3cn2)nc(-c2ccc(-c3ccccc3)c3ccccc23)n1 ; CC1(C)OB(c2cccc(-c3cccc(-n4c5ccccc5c5cc6ccccc6cc54)c3)c2)OC1(C)C ; "Pd(t-Bu3P)2, K2CO3(aq.)" ; "THF" ; c1ccc(-c2ccc(-c3nc(-c4cccc(-c5cccc(-n6c7ccccc7c7cc8ccccc8cc76)c5)c4)nc(-c4cc5oc6ccccc6c5cn4)n3)c3ccccc23)cc1]

im-7-8 (15 g, 30.9 mmol) and im-g-3 (16.1 g, 32.5 mmol) were added to 300 ml of THF, and the mixture was stirred and refluxed. Then, potassium carbonate (12.8 g, 92.8 mmol) was dissolved in 100 ml of water and added thereto, and the mixture was stirred sufficiently and bis(tri-tert-butylphosphine)palladium(0) (0.2 g, 0.3 mmol) was added. After the reaction for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 17.9 g of Compound 95. (Yield: 71%, MS: [M+H]+=818)

### Synthesis Example 96

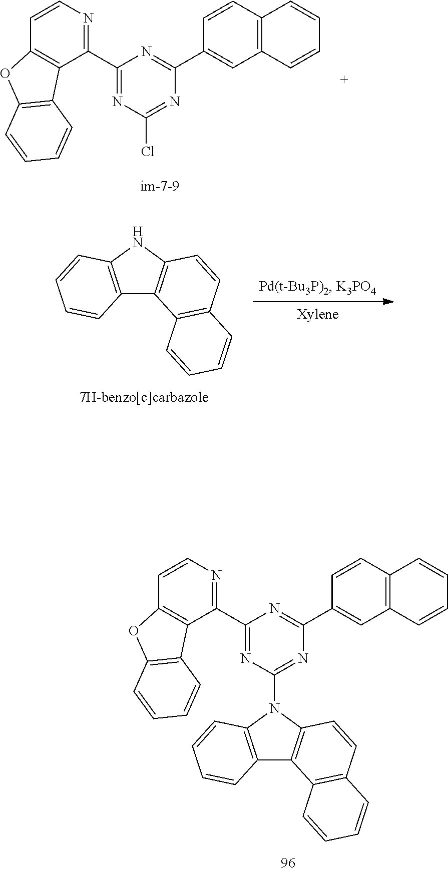

im-7-9 (15 g, 28.6 mmol), 7H-benzo[c]carbazole (6.5 g, 30 mmol) and potassium phosphate (18.2 g, 85.7 mmol) were added to 300 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 10.4 g of Compound 96. (Yield: 62%, MS: [M+H]+=590)

### Synthesis Example 97

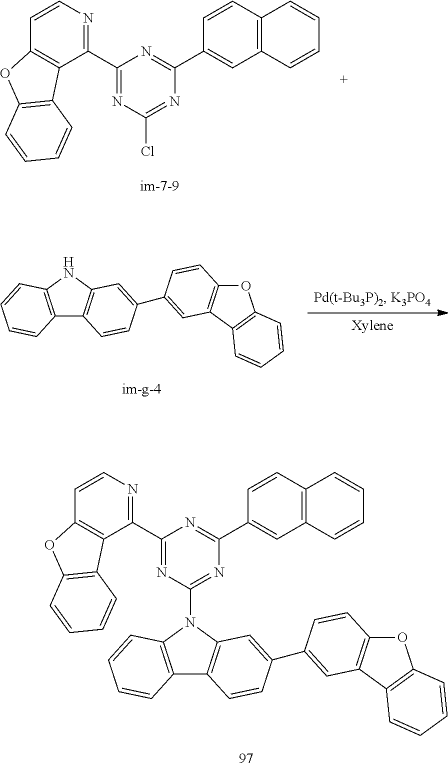

im-7-9 (15 g, 36.7 mmol), im-g-4 (12.8 g, 38.5 mmol) and potassium phosphate (23.4 g, 110.1 mmol) were added to 300 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 16.8 g of Compound 97. (Yield: 65%, MS: [M+H]+=706)

### Synthesis Example 98

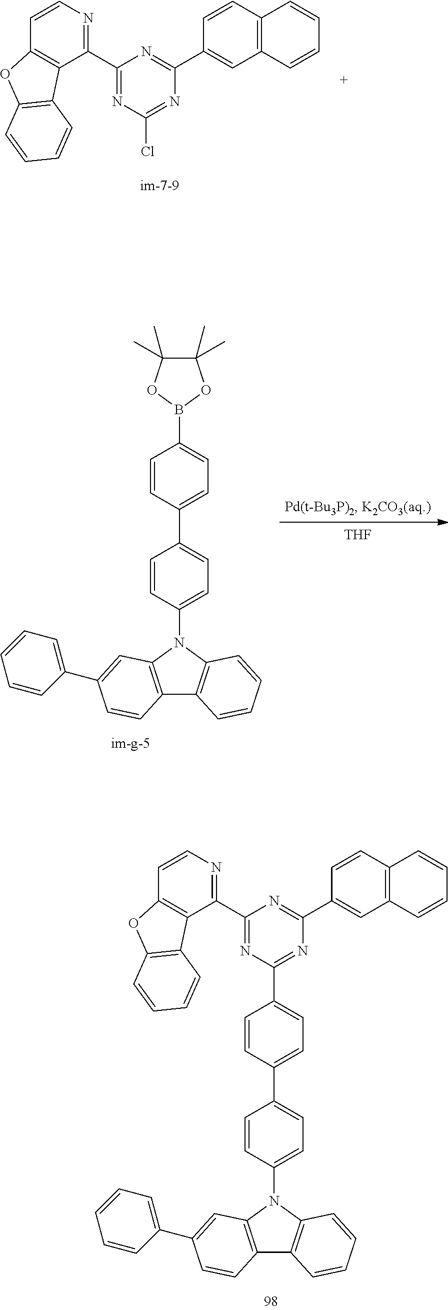 [SMILES: Clc1nc(-c2ccc3ccccc3c2)nc(-c2nccc3oc4ccccc4c23)n1 ; CC1(C)OB(c2ccc(-c3ccc(-n4c5ccccc5c5ccc(-c6ccccc6)cc54)cc3)cc2)OC1(C)C ; "Pd(t-Bu3P)2, K2CO3(aq.)" ; "THF" ; c1ccc(-c2ccc3c4ccccc4n(-c4ccc(-c5ccc(-c6nc(-c7ccc8ccccc8c7)nc(-c7nccc8oc9ccccc9c78)n6)cc5)cc4)c3c2)cc1]

im-7-9 (15 g, 36.7 mmol) and im-g-5 (20.1 g, 38.5 mmol) were added to 300 ml of THF, and the mixture was stirred and refluxed. Then, potassium carbonate (15.2 g, 110.1 mmol) was dissolved in 100 ml of water and added thereto, and the mixture was sufficiently stirred and bis(tri-tert-butylphosphine)palladium(0) (0.2 g, 0.4 mmol) was added. After the reaction for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 18 g of Compound 98. (Yield: 64%, MS: [M+H]+=768)

### Synthesis Example 99

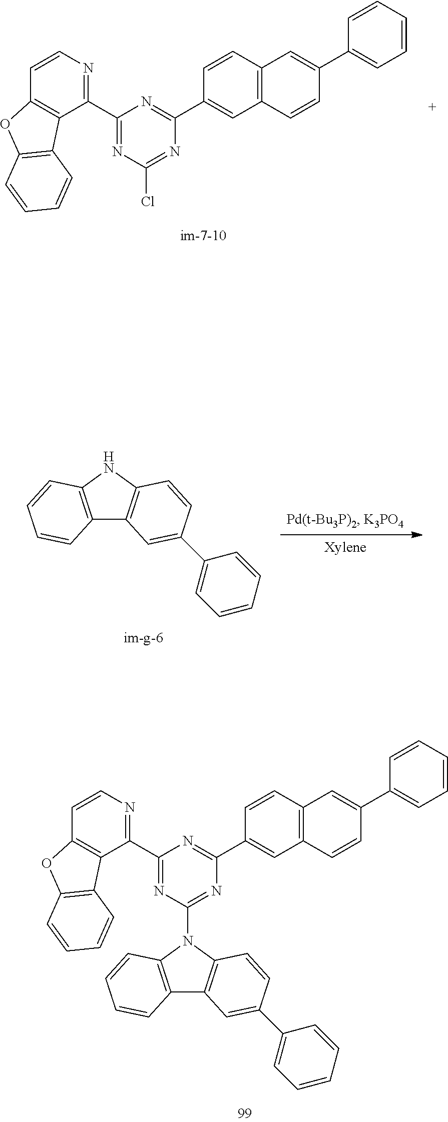

im-7-10 (15 g, 30.9 mmol), im-g-6 (7.9 g, 32.5 mmol) and potassium phosphate (19.7 g, 92.8 mmol) were added to 300 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 14.8 g of Compound 99. (Yield: 69%, MS: [M+H]+=692)

### Synthesis Example 100

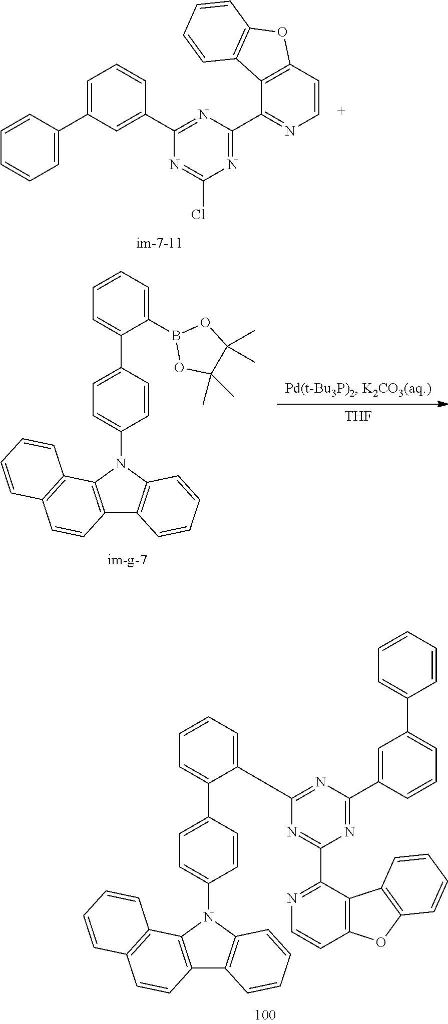 [SMILES: Clc1nc(-c2cccc(-c3ccccc3)c2)nc(-c2nccc3oc4ccccc4c23)n1 ; CC1(C)OB(c2ccccc2-c2ccc(-n3c4ccccc4c4ccc5ccccc5c43)cc2)OC1(C)C ; "Pd(t-Bu3P)2, K2CO3(aq.)" ; "THF" ; c1ccc(-c2cccc(-c3nc(-c4ccccc4-c4ccc(-n5c6ccccc6c6ccc7ccccc7c65)cc4)nc(-c4nccc5oc6ccccc6c45)n3)c2)cc1]

im-7-11 (15 g, 34.5 mmol) and im-g-7 (17.9 g, 36.2 mmol) were added to 300 ml of THF, and the mixture was stirred and refluxed. Then, potassium carbonate (14.3 g, 103.5 mmol) was dissolved in 100 ml of water and added thereto, and the mixture was sufficiently stirred and bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.7 g of Compound 100. (Yield: 63%, MS: [M+H]+=768)

### EXAMPLE

### Example 1

A glass substrate on which a thin film of ITO (indium tin oxide) was coated in a thickness of 1,000 Å was put into distilled water containing the detergent dissolved therein and washed by the ultrasonic wave. In this case, the used detergent was a product commercially available from Fisher Co. and the distilled water was one which had been twice filtered by using a filter commercially available from Millipore Co. The ITO was washed for 30 minutes, and ultrasonic washing was then repeated twice for 10 minutes by using distilled water. After the washing with distilled water was completed, the substrate was ultrasonically washed with isopropyl alcohol, acetone, and methanol solvent, and dried, after which it was transported to a plasma cleaner. Then, the substrate was cleaned with oxygen plasma for 5 minutes, and then transferred to a vacuum evaporator.

On the ITO transparent electrode thus prepared, the following Compound HI-1 was formed to a thickness of 1150 Å as a hole injection layer, and the following Compound A-1 was p-doped at a concentration of 1.5 wt. %. The following Compound HT-1 was vacuum deposited on the hole injection layer to form a hole transport layer with a film thickness of 800 Å. Then, the following Compound HT-2 was vacuum deposited on the hole transport layer to a film thickness of 150 Å to form a second hole transport layer. Then, Compound 1 prepared in Synthesis Example 1 as a host, and the following Compound Dp-7 as a dopant were vacuum deposited at a weight ratio of 98:2 on the second hole transport layer to form a light emitting layer with a thickness of 400 Å. The following Compound HB-1 was vacuum deposited on the light emitting layer to a film thickness of 30 Å to form a hole blocking layer. Then, the following Compound ET-1 and the following Compound LiQ were vacuum deposited at a weight ratio of 2:1 on the hole blocking layer to form an electron injection and transport layer with a film thickness of 300 Å. Lithium fluoride (LiF) and aluminum were sequentially deposited to have a thickness of 12 Å and 1,000 Å, respectively, on the electron injection and transport layer, thereby forming a cathode.

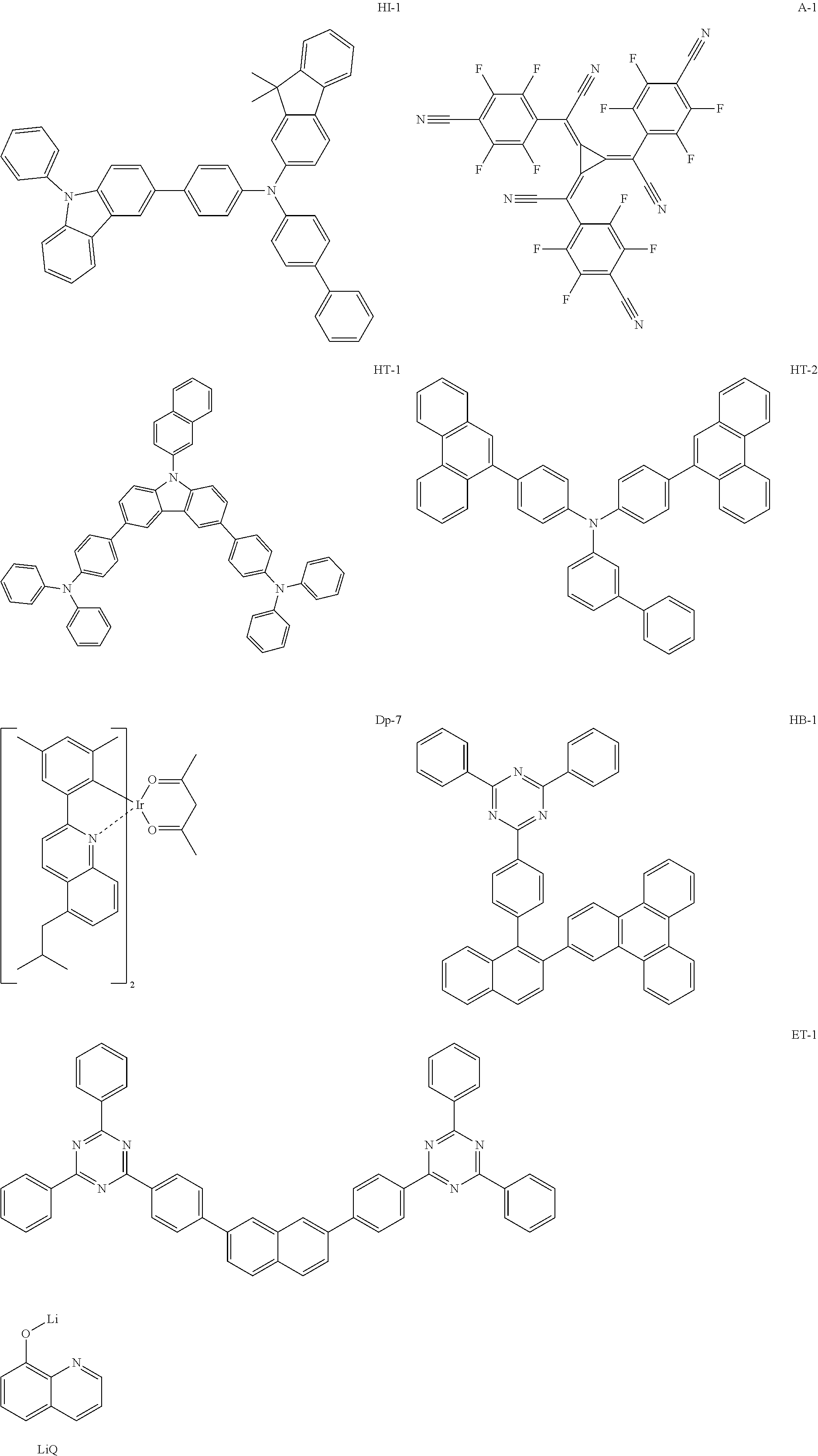

In the above-mentioned processes, the deposition rates of the organic materials were maintained at 0.4˜0.7 Å/sec, the deposition rates of lithium fluoride and the aluminum of the cathode were maintained at 0.3 Å/sec and 2 Å/sec, respectively, and the degree of vacuum during the deposition was maintained at 2×10⁻⁷˜5×10⁻⁶ torr, thereby manufacturing an organic light emitting device.

### Examples 2 to 100

The organic light emitting devices were manufactured in the same manner as in Example 1, except that in the organic light emitting device of Example 1, the compounds shown in Table 1 below were used instead of Compound 1.

### Comparative Examples 1 to 12

The organic light emitting devices were manufactured in the same manner as in Example 1, except that in the organic light emitting device of Example 1, the compounds shown in Table 1 below were used instead of Compound 1. In Table 1, the Compounds C-1 to C-12 are as follows:

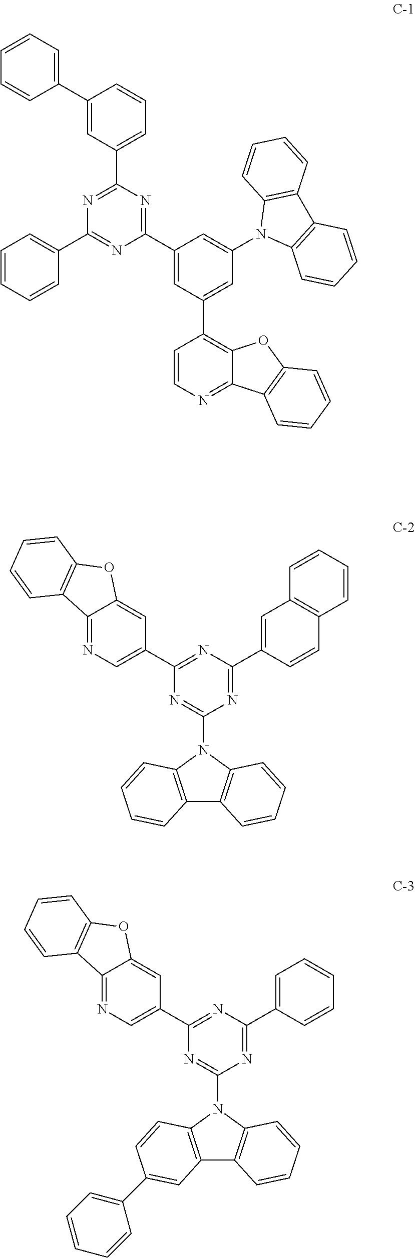 [SMILES: c1ccc(-c2cccc(-c3nc(-c4ccccc4)nc(-c4cc(-c5ccnc6c5oc5ccccc56)cc(-n5c6ccccc6c6ccccc65)c4)n3)c2)cc1 ; c1ccc2cc(-c3nc(-c4cnc5c(c4)oc4ccccc45)nc(-n4c5ccccc5c5ccccc54)n3)ccc2c1 ; c1ccc(-c2ccc3c(c2)c2ccccc2n3-c2nc(-c3ccccc3)nc(-c3cnc4c(c3)oc3ccccc34)n2)cc1]

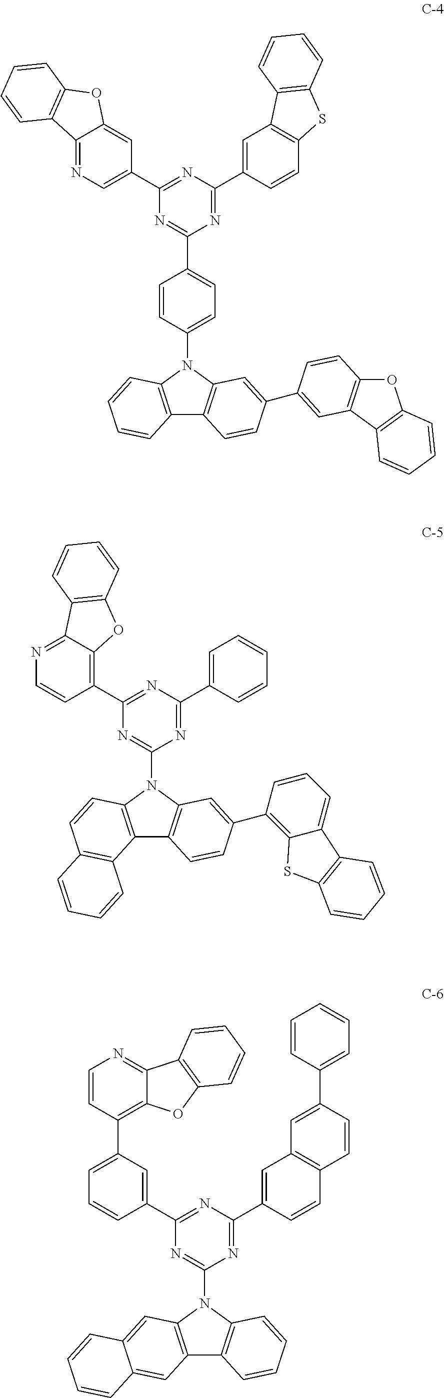 [SMILES: c1ccc2c(c1)oc1ccc(-c3ccc4c5ccccc5n(-c5ccc(-c6nc(-c7cnc8c(c7)oc7ccccc78)nc(-c7ccc8sc9ccccc9c8c7)n6)cc5)c4c3)cc12 ; c1ccc(-c2nc(-c3ccnc4c3oc3ccccc34)nc(-n3c4cc(-c5cccc6c5sc5ccccc56)ccc4c4c5ccccc5ccc43)n2)cc1 ; c1ccc(-c2ccc3ccc(-c4nc(-c5cccc(-c6ccnc7c6oc6ccccc67)c5)nc(-n5c6ccccc6c6cc7ccccc7cc65)n4)cc3c2)cc1]

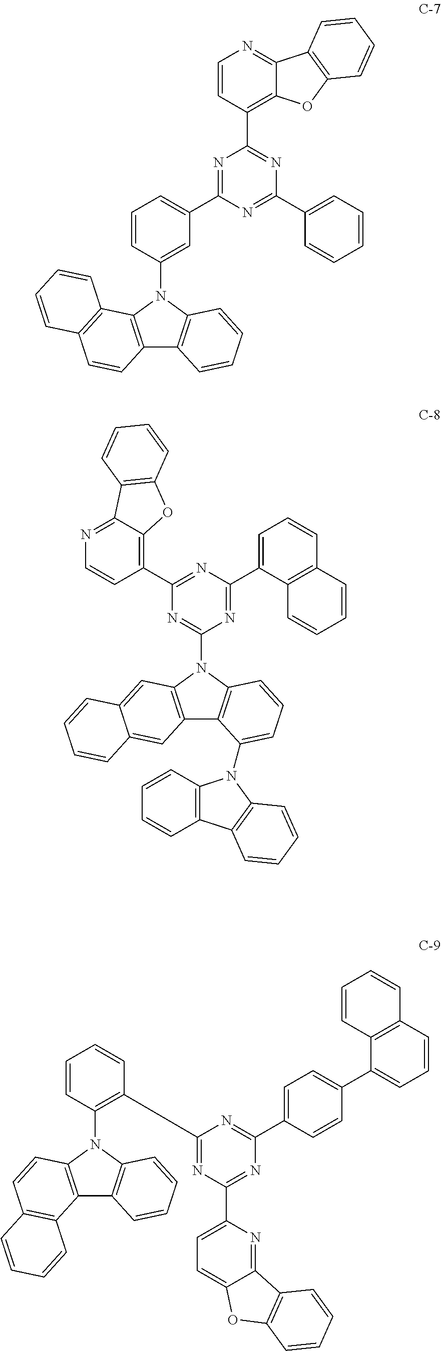 [SMILES: c1ccc(-c2nc(-c3cccc(-n4c5ccccc5c5ccc6ccccc6c54)c3)nc(-c3ccnc4c3oc3ccccc34)n2)cc1 ; c1ccc2cc3c(cc2c1)c1c(-n2c4ccccc4c4ccccc42)cccc1n3-c1nc(-c2cccc3ccccc23)nc(-c2ccnc3c2oc2ccccc23)n1 ; c1ccc(-n2c3ccccc3c3c4ccccc4ccc32)c(-c2nc(-c3ccc(-c4cccc5ccccc45)cc3)nc(-c3ccc4oc5ccccc5c4n3)n2)c1]

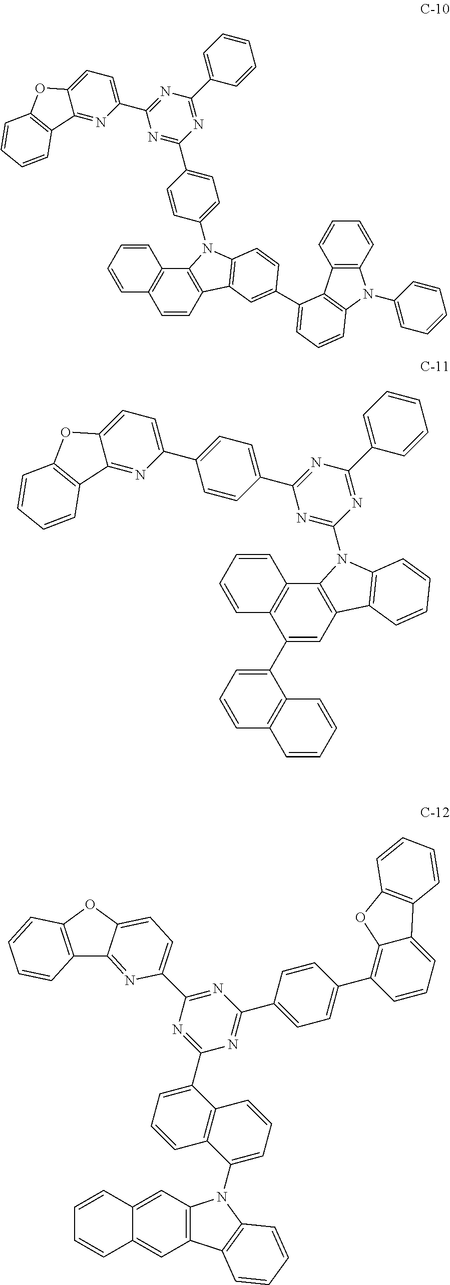 [SMILES: c1ccc(-c2nc(-c3ccc(-n4c5ccc(-c6cccc7c6c6ccccc6n7-c6ccccc6)cc5c5ccc6ccccc6c54)cc3)nc(-c3ccc4oc5ccccc5c4n3)n2)cc1 ; c1ccc(-c2nc(-c3ccc(-c4ccc5oc6ccccc6c5n4)cc3)nc(-n3c4ccccc4c4cc(-c5cccc6ccccc56)c5ccccc5c43)n2)cc1 ; c1ccc2cc3c(cc2c1)c1ccccc1n3-c1cccc2c(-c3nc(-c4ccc(-c5cccc6c5oc5ccccc56)cc4)nc(-c4ccc5oc6ccccc6c5n4)n3)cccc12]

The voltage and efficiency were measured (15 mA/cm²) by applying a current to the organic light emitting devices manufactured in Examples 1 to 100 and Comparative Examples 1 to 12, and the results are shown in Table 1 below. Lifetime T95 means the time required for the luminance to be reduced to 95% of the initial luminance (6000 nit).

TABLE 1

| | | Driving | | | |
|---|---|---|---|---|---|
| | | voltage | Efficiency | Lifetime | Luminous |
| Category | Material | (V) | (cd/A) | T95(hr) | color |
| Example 1 | Compound 1 | 3.73 | 19.21 | 142 | Red |
| Example 2 | Compound 2 | 3.60 | 19.68 | 152 | Red |
| Example 3 | Compound 3 | 3.61 | 20.43 | 150 | Red |
| Example 4 | Compound 4 | 3.56 | 19.80 | 148 | Red |
| Example 5 | Compound 5 | 3.62 | 20.02 | 146 | Red |
| Example 6 | Compound 6 | 3.65 | 20.46 | 148 | Red |
| Example 7 | Compound 7 | 3.53 | 20.77 | 163 | Red |
| Example 8 | Compound 8 | 3.49 | 21.02 | 172 | Red |
| Example 9 | Compound 9 | 3.56 | 21.33 | 168 | Red |
| Example 10 | Compound | 3.57 | 20.96 | 160 | Red |
| | 10 | | | | |
| Example 11 | Compound | 3.57 | 21.17 | 147 | Red |
| | 11 | | | | |
| Example 12 | Compound | 3.56 | 21.42 | 167 | Red |
| | 12 | | | | |
| Example 13 | Compound | 3.69 | 19.29 | 154 | Red |
| | 13 | | | | |
| Example 14 | Compound | 3.68 | 19.27 | 140 | Red |
| | 14 | | | | |
| Example 15 | Compound | 3.63 | 19.75 | 153 | Red |
| | 15 | | | | |
| Example 16 | Compound | 3.57 | 20.07 | 145 | Red |
| | 16 | | | | |
| Example 17 | Compound | 3.66 | 19.62 | 148 | Red |
| | 17 | | | | |
| Example 18 | Compound | 3.64 | 19.95 | 141 | Red |
| | 18 | | | | |
| Example 19 | Compound | 3.64 | 20.30 | 140 | Red |
| | 19 | | | | |
| Example 20 | Compound | 3.68 | 20.09 | 152 | Red |
| | 20 | | | | |
| Example 21 | Compound | 3.60 | 19.60 | 154 | Red |
| | 21 | | | | |
| Example 22 | Compound | 3.76 | 19.60 | 175 | Red |
| | 22 | | | | |
| Example 23 | Compound | 3.59 | 20.45 | 166 | Red |
| | 23 | | | | |
| Example 24 | Compound | 3.69 | 19.46 | 166 | Red |
| | 24 | | | | |
| Example 25 | Compound | 3.75 | 20.40 | 161 | Red |
| | 25 | | | | |
| Example 26 | Compound | 3.63 | 19.43 | 160 | Red |
| | 26 | | | | |
| Example 27 | Compound | 3.65 | 19.93 | 167 | Red |
| | 27 | | | | |
| Example 28 | Compound | 3.67 | 20.40 | 161 | Red |
| | 28 | | | | |
| Example 29 | Compound | 3.56 | 19.53 | 165 | Red |
| | 29 | | | | |
| Example 30 | Compound | 3.48 | 21.16 | 173 | Red |
| | 30 | | | | |
| Example 31 | Compound | 3.54 | 20.55 | 175 | Red |
| | 31 | | | | |
| Example 32 | Compound | 3.50 | 21.32 | 166 | Red |
| | 32 | | | | |
| Example 33 | Compound | 3.47 | 21.13 | 164 | Red |
| | 33 | | | | |
| Example 34 | Compound | 3.60 | 21.12 | 168 | Red |
| | 34 | | | | |
| Example 35 | Compound | 3.48 | 20.74 | 167 | Red |
| | 35 | | | | |
| Example 36 | Compound | 3.47 | 21.28 | 168 | Red |
| | 36 | | | | |
| Example 37 | Compound | 3.57 | 20.66 | 165 | Red |
| | 37 | | | | |
| Example 38 | Compound | 3.34 | 21.28 | 189 | Red |
| | 38 | | | | |
| Example 39 | Compound | 3.38 | 21.77 | 188 | Red |
| | 39 | | | | |
| Example 40 | Compound | 3.39 | 22.38 | 175 | Red |
| | 40 | | | | |
| Example 41 | Compound | 3.44 | 21.25 | 181 | Red |
| | 41 | | | | |
| Example 42 | Compound | 3.47 | 21.89 | 184 | Red |
| | 42 | | | | |
| Example 43 | Compound | 3.40 | 21.93 | 182 | Red |
| | 43 | | | | |
| Example 44 | Compound | 3.49 | 22.12 | 181 | Red |
| | 44 | | | | |
| Example 45 | Compound | 3.35 | 21.83 | 171 | Red |
| | 45 | | | | |
| Example 46 | Compound | 3.47 | 21.85 | 186 | Red |
| | 46 | | | | |
| Example 47 | Compound | 3.47 | 21.44 | 180 | Red |
| | 47 | | | | |
| Example 48 | Compound | 3.38 | 21.73 | 173 | Red |
| | 48 | | | | |
| Example 49 | Compound | 3.34 | 20.80 | 171 | Red |
| | 49 | | | | |
| Example 50 | Compound | 3.35 | 20.34 | 183 | Red |
| | 50 | | | | |
| Example 51 | Compound | 3.59 | 20.03 | 152 | Red |
| | 51 | | | | |
| Example 52 | Compound | 3.74 | 20.31 | 142 | Red |
| | 52 | | | | |
| Example 53 | Compound | 3.67 | 19.88 | 169 | Red |
| | 53 | | | | |
| Example 54 | Compound | 3.66 | 19.84 | 165 | Red |
| | 54 | | | | |
| Example 55 | Compound | 3.69 | 19.49 | 163 | Red |
| | 55 | | | | |
| Example 56 | Compound | 3.74 | 19.80 | 169 | Red |
| | 56 | | | | |
| Example 57 | Compound | 3.56 | 19.47 | 170 | Red |
| | 57 | | | | |
| Example 58 | Compound | 3.69 | 20.13 | 150 | Red |
| | 58 | | | | |
| Example 59 | Compound | 3.63 | 20.09 | 148 | Red |
| | 59 | | | | |
| Example 60 | Compound | 3.72 | 20.47 | 141 | Red |
| | 60 | | | | |
| Example 61 | Compound | 3.69 | 19.40 | 148 | Red |
| | 61 | | | | |
| Example 62 | Compound | 3.63 | 19.99 | 154 | Red |
| | 62 | | | | |
| Example 63 | Compound | 3.56 | 21.29 | 162 | Red |
| | 63 | | | | |
| Example 64 | Compound | 3.53 | 21.02 | 163 | Red |
| | 64 | | | | |
| Example 65 | Compound | 3.67 | 19.14 | 155 | Red |
| | 65 | | | | |
| Example 66 | Compound | 3.74 | 19.41 | 148 | Red |
| | 66 | | | | |
| Example 67 | Compound | 3.66 | 21.39 | 182 | Red |
| | 67 | | | | |
| Example 68 | Compound | 3.69 | 20.52 | 171 | Red |
| | 68 | | | | |
| Example 69 | Compound | 3.57 | 19.47 | 163 | Red |
| | 69 | | | | |
| Example 70 | Compound | 3.56 | 19.51 | 171 | Red |
| | 70 | | | | |
| Example 71 | Compound | 3.58 | 20.10 | 173 | Red |
| | 71 | | | | |
| Example 72 | Compound | 3.56 | 19.92 | 173 | Red |
| | 72 | | | | |
| Example 73 | Compound | 3.61 | 20.46 | 163 | Red |
| | 73 | | | | |
| Example 74 | Compound | 3.56 | 19.77 | 170 | Red |
| | 74 | | | | |
| Example 75 | Compound | 3.59 | 19.83 | 167 | Red |
| | 75 | | | | |
| Example 76 | Compound | 3.57 | 20.37 | 175 | Red |
| | 76 | | | | |
| Example 77 | Compound | 3.65 | 20.36 | 149 | Red |
| | 77 | | | | |
| Example 78 | Compound | 3.78 | 19.03 | 142 | Red |
| | 78 | | | | |
| Example 79 | Compound | 3.51 | 20.51 | 169 | Red |
| | 79 | | | | |
| Example 80 | Compound | 3.59 | 21.37 | 160 | Red |
| | 80 | | | | |
| Example 81 | Compound | 3.48 | 20.58 | 160 | Red |
| | 81 | | | | |
| Example 82 | Compound | 3.55 | 21.40 | 172 | Red |
| | 82 | | | | |
| Example 83 | Compound | 3.55 | 20.58 | 174 | Red |
| | 83 | | | | |
| Example 84 | Compound | 3.57 | 20.21 | 168 | Red |
| | 84 | | | | |
| Example 85 | Compound | 3.48 | 20.99 | 161 | Red |
| | 85 | | | | |
| Example 86 | Compound | 3.60 | 20.20 | 165 | Red |
| | 86 | | | | |
| Example 87 | Compound | 3.55 | 20.89 | 171 | Red |
| | 87 | | | | |
| Example 88 | Compound | 3.44 | 20.46 | 170 | Red |
| | 88 | | | | |
| Example 89 | Compound | 3.33 | 20.57 | 190 | Red |
| | 89 | | | | |
| Example 90 | Compound | 3.47 | 20.26 | 172 | Red |
| | 90 | | | | |
| Example 91 | Compound | 3.65 | 20.46 | 169 | Red |
| | 91 | | | | |
| Example 92 | Compound | 3.67 | 19.40 | 146 | Red |
| | 92 | | | | |
| Example 93 | Compound | 3.64 | 19.68 | 141 | Red |
| | 93 | | | | |
| Example 94 | Compound | 3.55 | 19.62 | 151 | Red |
| | 94 | | | | |
| Example 95 | Compound | 3.69 | 19.53 | 150 | Red |
| | 95 | | | | |
| Example 96 | Compound | 3.50 | 21.24 | 165 | Red |
| | 96 | | | | |
| Example 97 | Compound | 3.47 | 21.41 | 170 | Red |
| | 97 | | | | |
| Example 98 | Compound | 3.60 | 20.52 | 166 | Red |
| | 98 | | | | |
| Example 99 | Compound | 3.51 | 20.90 | 162 | Red |
| | 99 | | | | |
| Example | Compound | 3.56 | 21.10 | 171 | Red |
| 100 | 100 | | | | |
| Comparative | C-1 | 4.08 | 17.03 | 74 | Red |
| Example 1 | | | | | |
| Comparative | C-2 | 3.91 | 18.06 | 97 | Red |
| Example 2 | | | | | |
| Comparative | C-3 | 3.93 | 17.91 | 93 | Red |
| Example 3 | | | | | |
| Comparative | C-4 | 3.99 | 17.36 | 86 | Red |
| Example 4 | | | | | |
| Comparative | C-5 | 3.94 | 18.03 | 121 | Red |
| Example 5 | | | | | |
| Comparative | C-6 | 3.96 | 18.12 | 128 | Red |
| Example 6 | | | | | |
| Comparative | C-7 | 4.01 | 17.59 | 116 | Red |
| Example 7 | | | | | |
| Comparative | C-8 | 4.15 | 17.11 | 85 | Red |
| Example 8 | | | | | |
| Comparative | C-9 | 3.93 | 17.86 | 108 | Red |
| Example 9 | | | | | |
| Comparative | C-10 | 4.06 | 17.57 | 96 | Red |
| Example 10 | | | | | |
| Comparative | C-11 | 3.92 | 16.42 | 83 | Red |
| Example 11 | | | | | |
| Comparative | C-12 | 4.04 | 16.13 | 105 | Red |
| Example 12 | | | | | |

As shown in Table 1, when a current was applied to the organic light emitting devices manufactured in Examples 1 to 100 and Comparative Examples 1 to 12, the results shown in Table 1 were obtained.

The red organic light emitting device of Example 1 has a structure in which a conventionally widely used material was used, and Dp-7 was used as a dopant for the red light emitting layer. In Comparative Examples 1 to 14, organic light emitting devices were manufactured by using C-1 to C-12 instead of Compound 1. Looking at the results in Table 1, when the compound of the present disclosure was used as the host compound for the light emitting layer, the performance was generally improved as compared to the materials of Comparative Examples, and from these results, it was found that the energy transfer from the host to the red dopant was well achieved, particularly, the lifetime characteristic was improved. It can be judged that this is because the compounds of the present disclosure have higher stability to electrons and holes than the compounds of Comparative Examples.

In conclusion, it can be confirmed that when the compound of the present disclosure was used as a host for the red light emitting layer, the driving voltage, luminous efficiency, and lifetime characteristics of the organic light emitting devices could be improved.

### DESCRIPTION OF REFERENCE NUMERALS

1: substrate
2: anode
3: light emitting layer
4: cathode
5: hole injection layer
6: hole transport layer
7: second hole transport layer
8: hole blocking layer
9: electron injection and transport layer

1. A compound represented by the following Chemical Formula 1: 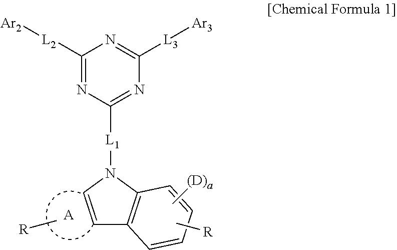
in Chemical Formula 1,
A is a substituted or unsubstituted benzene or naphthalene,
each R is independently hydrogen, deuterium, a substituted or unsubstituted C₆₋₆₀ aryl, or a substituted or unsubstituted C₂₋₆₀ heteroaryl containing one or more selected from the group consisting of N, O and S,
D is deuterium,
a is an integer of 0 to 3,
L₁ is a single bond, phenylene, biphenyldiyl, naphthalenediyl, (naphthalenediyl)(phenylene), (phenylene)(naphthalenediyl), or (naphthalenediyl)(naphthalenediyl),
wherein, when L₁ is phenylene, biphenyldiyl, naphthalenediyl, (naphthalenediyl)(phenylene), (phenylene)(naphthalenediyl), or (naphthalenediyl)(naphthalenediyl), L₁ is unsubstituted or substituted with at least one deuterium,
L₂ and L₃ are each independently a single bond, a substituted or unsubstituted C₆₋₆₀ arylene, or a substituted or unsubstituted C₂₋₆₀ heteroarylene containing one or more selected from the group consisting of N, O and S,
Ar₂ is a substituted or unsubstituted C₆₋₆₀ aryl, or a substituted or unsubstituted C₂₋₆₀ heteroaryl containing one or more selected from the group consisting of N, O and S, and
Ar₃ is a substituent represented by the following Chemical Formula 2, 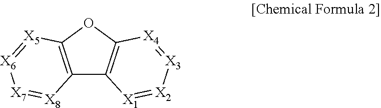
in Chemical Formula 2,
one of X₁ to X₈ is N, another one is C bonded to L₃, and the rest is CH or CD,
provided that when X₁ is N, one of X₅ to X₈ is C bonded to L₃, and when X₈ is N, one of X₁ to X₄ is C bonded to L₃.

2. The compound according to claim 1 wherein:
A is a benzene unsubstituted or substituted with 1 to 4 deuterium; or naphthalene unsubstituted or substituted with 1 to 6 deuterium.

3. The compound according to claim 1 wherein:
the compound of Chemical Formula 1 is represented by one of the following Chemical Formulas 1-1 to 1-4: 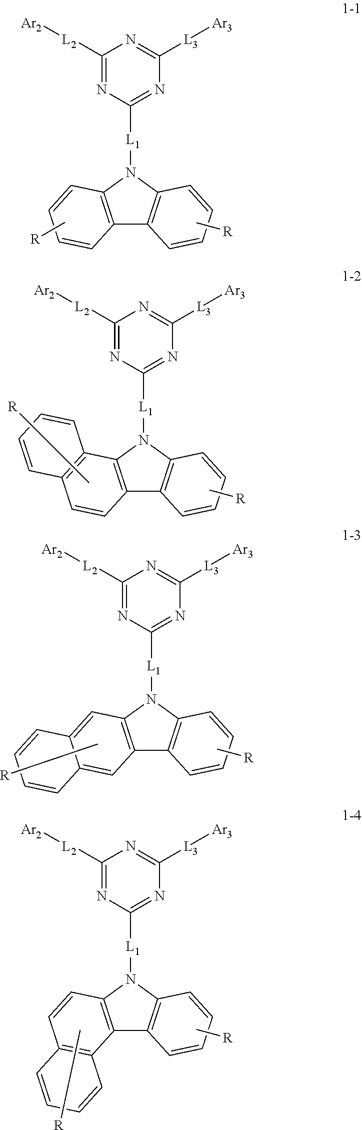
in Chemical Formulas 1-1 to 1-4,
R, L₁, L₂, L₃, Ar₂ and Ar₃ are the same as defined in claim 1.

4. The compound according to claim 1 wherein
each R is independently hydrogen, deuterium, phenyl, biphenylyl, naphthyl, dibenzofuranyl, dibenzothiophenyl, 9H-carbazol-9-yl, or 9-phenyl-9H-carbazolyl, each of which, except for hydrogen and deuterium,
is unsubstituted or substituted with at least one deuterium.

5. The compound according to claim 1 wherein:
each R is hydrogen; or
each R is deuterium; or
one R is hydrogen or deuterium, and the other R is phenyl, biphenylyl, naphthyl, dibenzofuranyl, dibenzothiophenyl, 9H-carbazol-9-yl, or 9-phenyl-9H-carbazolyl, each of which
is unsubstituted or substituted with at least one deuterium.

6. The compound according to claim 1 wherein:
L₁ is a single bond, phenylene, biphenyldiyl, or naphthalenediyl, each of which, except for a single bond,
is unsubstituted or substituted with at least one deuterium.

7. The compound according to claim 1 wherein:
L₂ and L₃ are each independently a single bond, phenylene, biphenyldiyl, or naphthylene, each of which, except for a single bond,
is unsubstituted or substituted with at least one deuterium.

8. The compound according to claim 1 wherein
L₃ is a single bond, phenylene, or naphthylene, each of which, except for a single bond,
is unsubstituted or substituted with at least one deuterium.

9. The compound according to claim 1 wherein
Ar₂ is phenyl, biphenylyl, terphenylyl, naphthyl, phenylnaphthyl, naphthylphenyl, phenanthrenyl, dimethylfluorenyl, diphenylfluorenyl, dibenzofuranyl, dibenzothiophenyl, 9H-carbazol-9-yl, or 9-phenyl-9H-carbazolyl, each of which
is unsubstituted or substituted with at least one deuterium or triphenylsilyl.

10. The compound according to claim 1 wherein
the substituent represented by Chemical Formula 2 is represented by one of the following Chemical Formulas 2a to 2d: 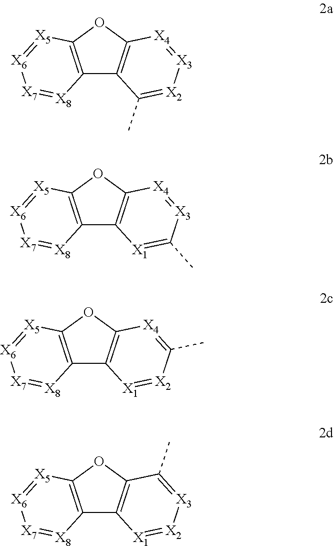
in Chemical Formula 2a, one of X₂ to X₄ and X₅ to X₈ is N, and the rest are each independently CH or CD,
in Chemical Formula 2b, one of X₁ and X₃ to X₈ is N, and the rest are each independently CH or CD,
in Chemical Formula 2c, one of X₁, X₂ and X₄ to X₈ is N, and the rest are each independently CH or CD, and
in Chemical Formula 2d, one of X₁ to X₃ and X₅ to X₈ is N, and the rest are each independently CH or CD.

11. The compound according to claim 1 wherein
the compound represented by Chemical Formula 1 is one selected from the following compounds:

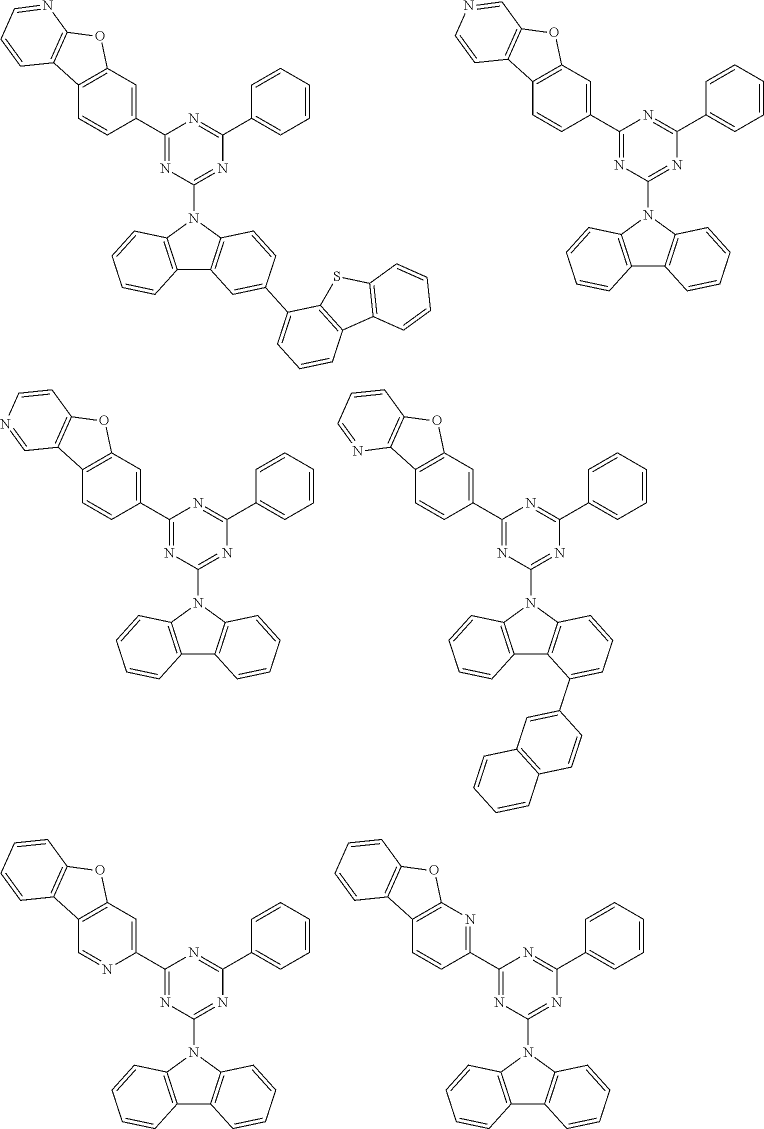

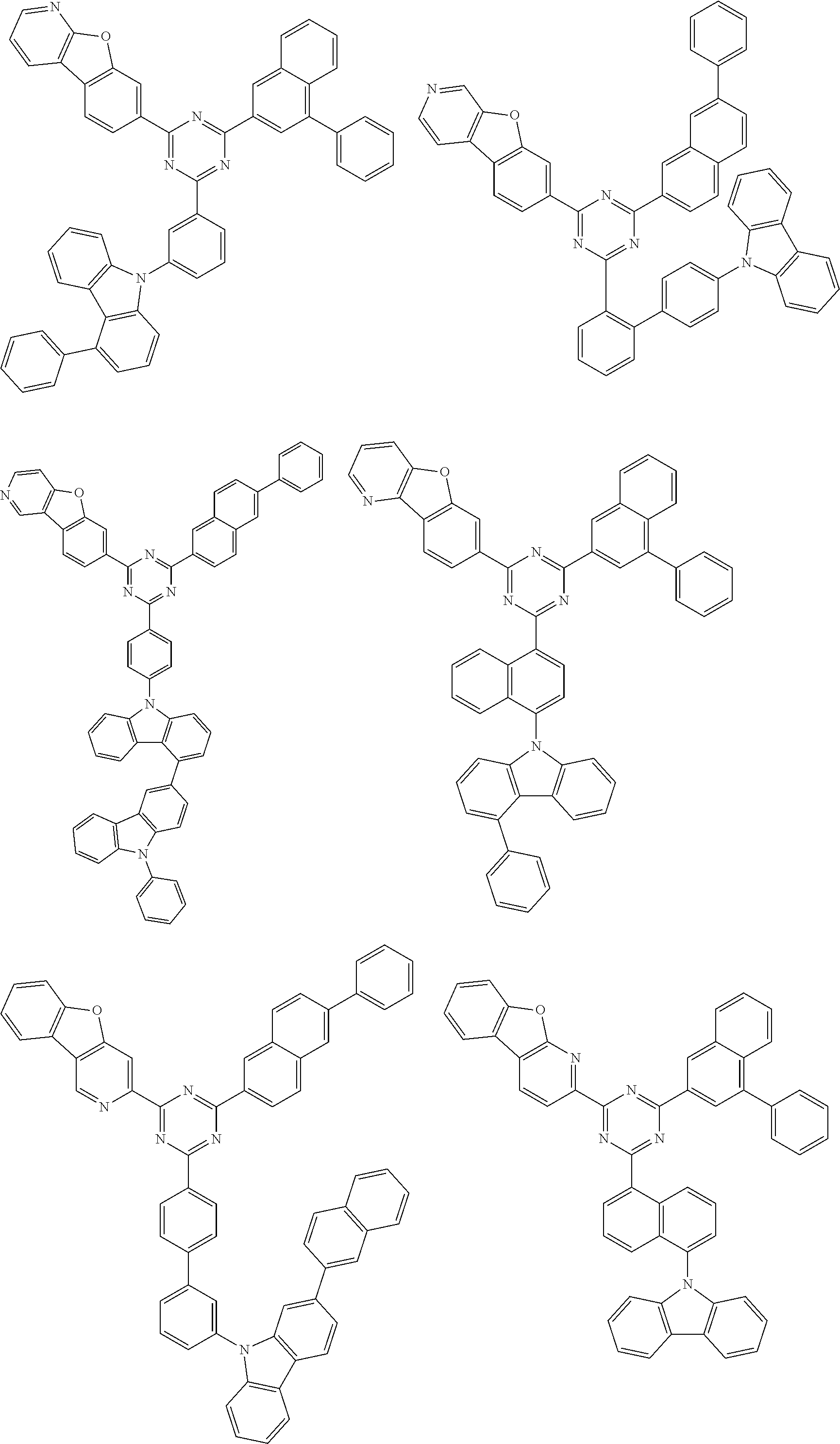

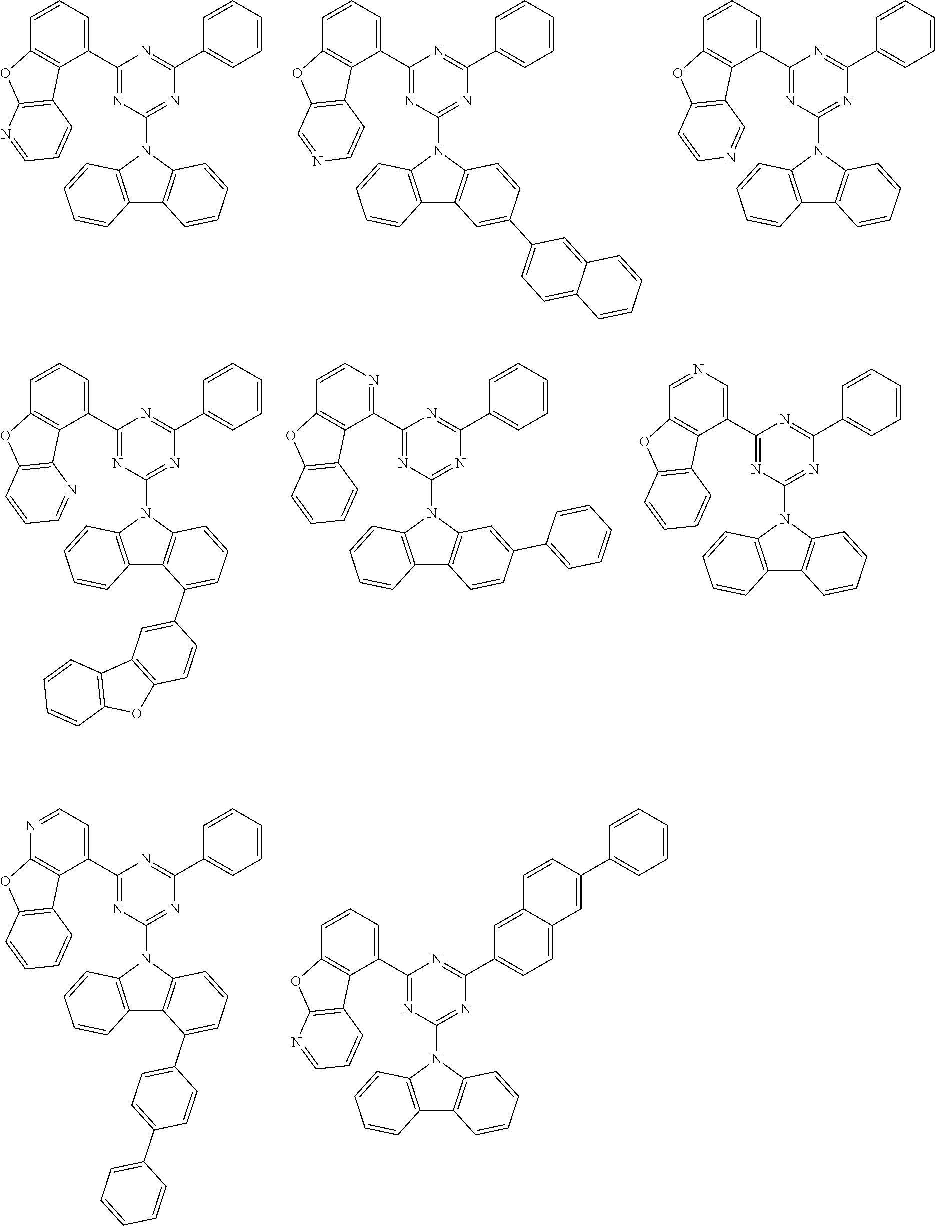

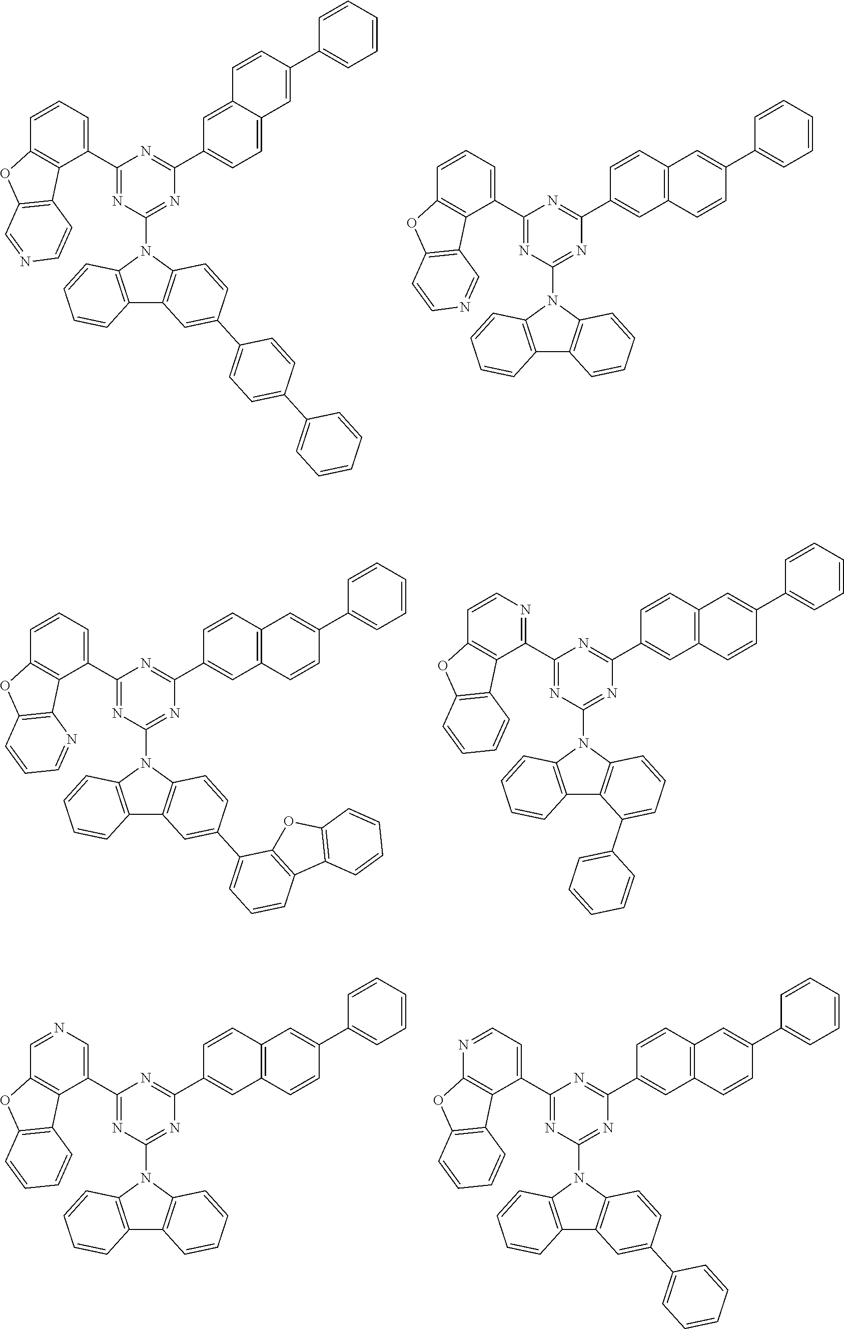

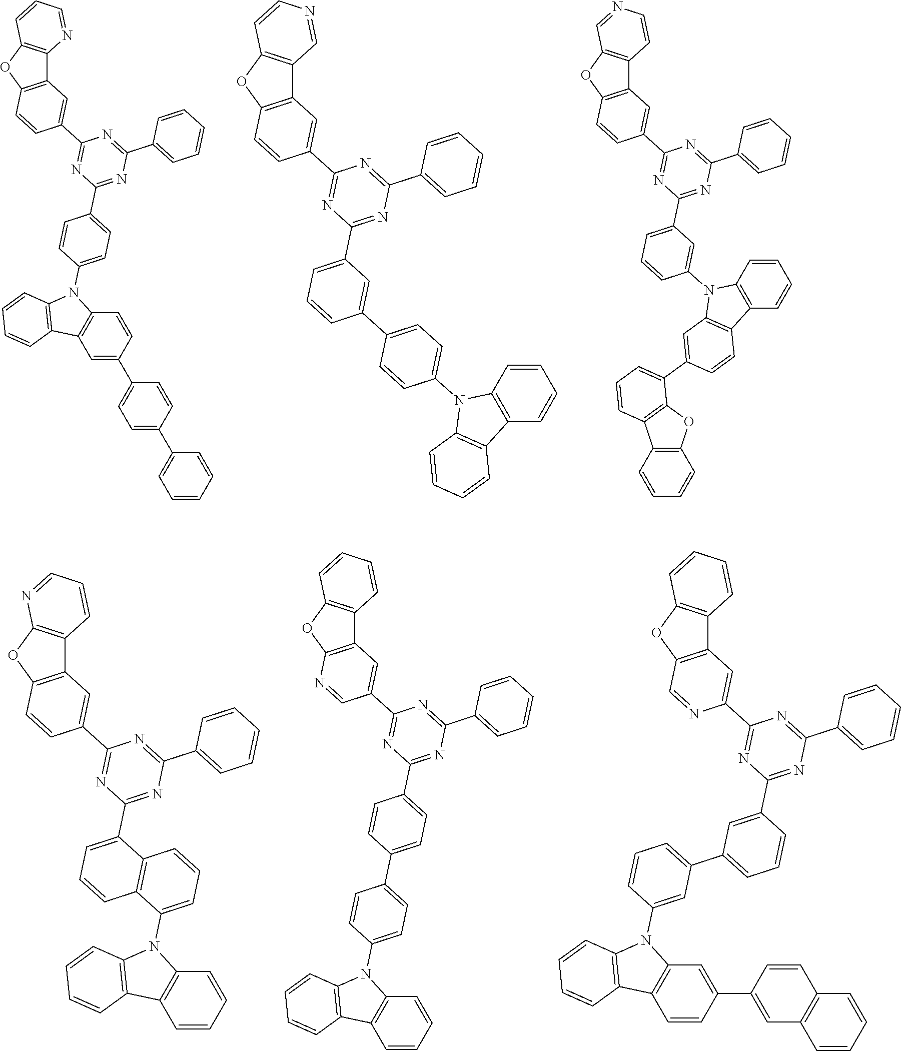

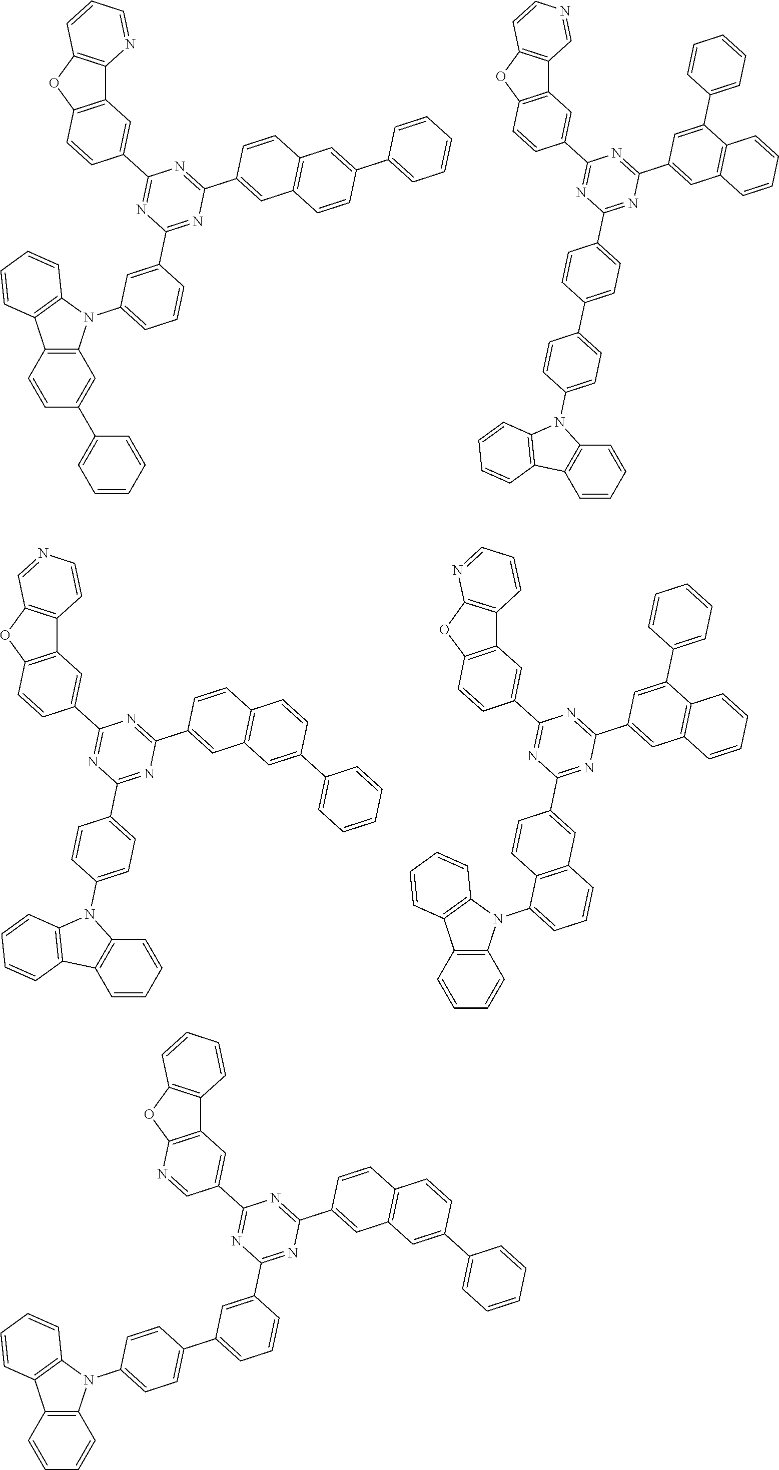

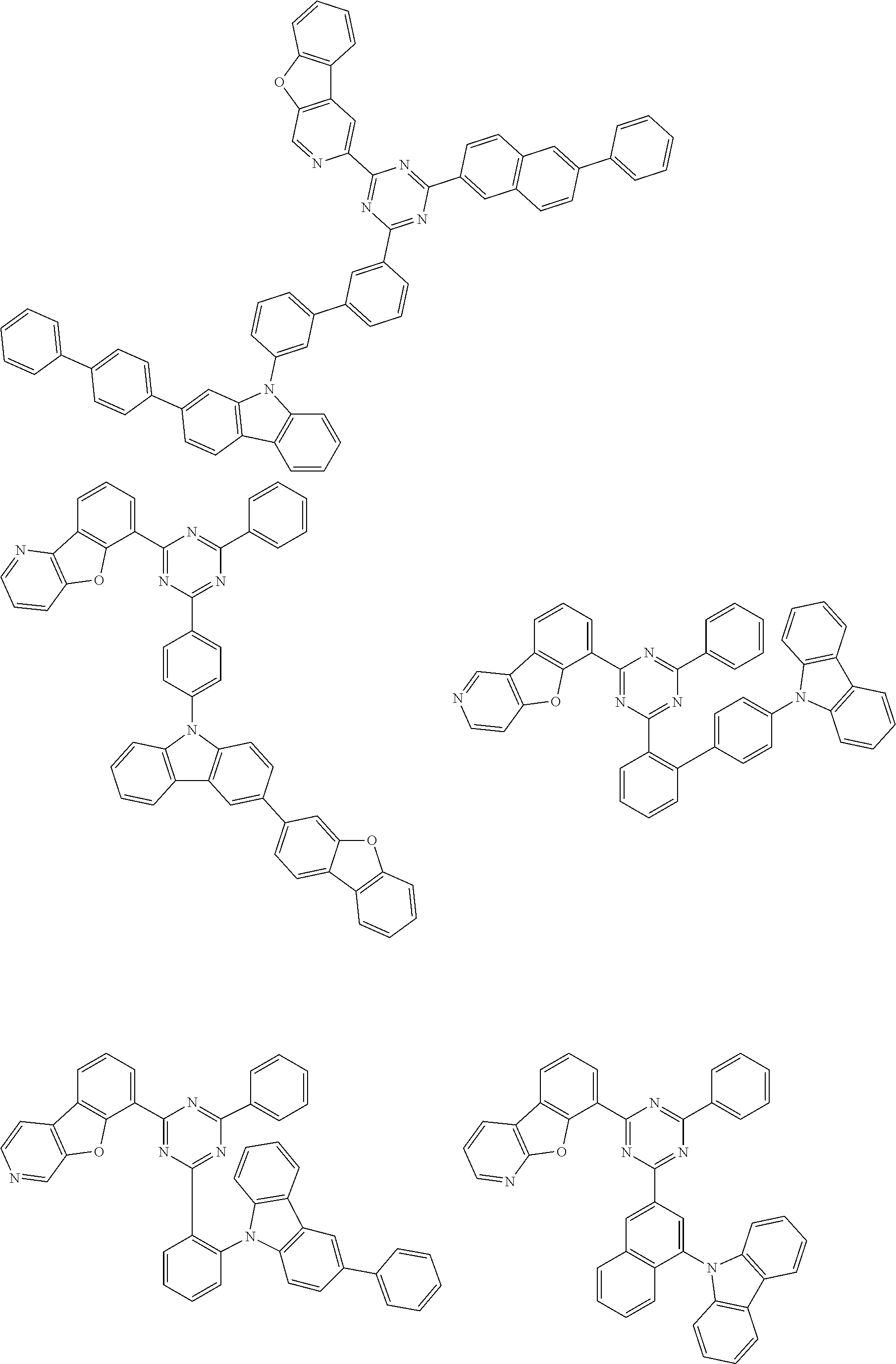

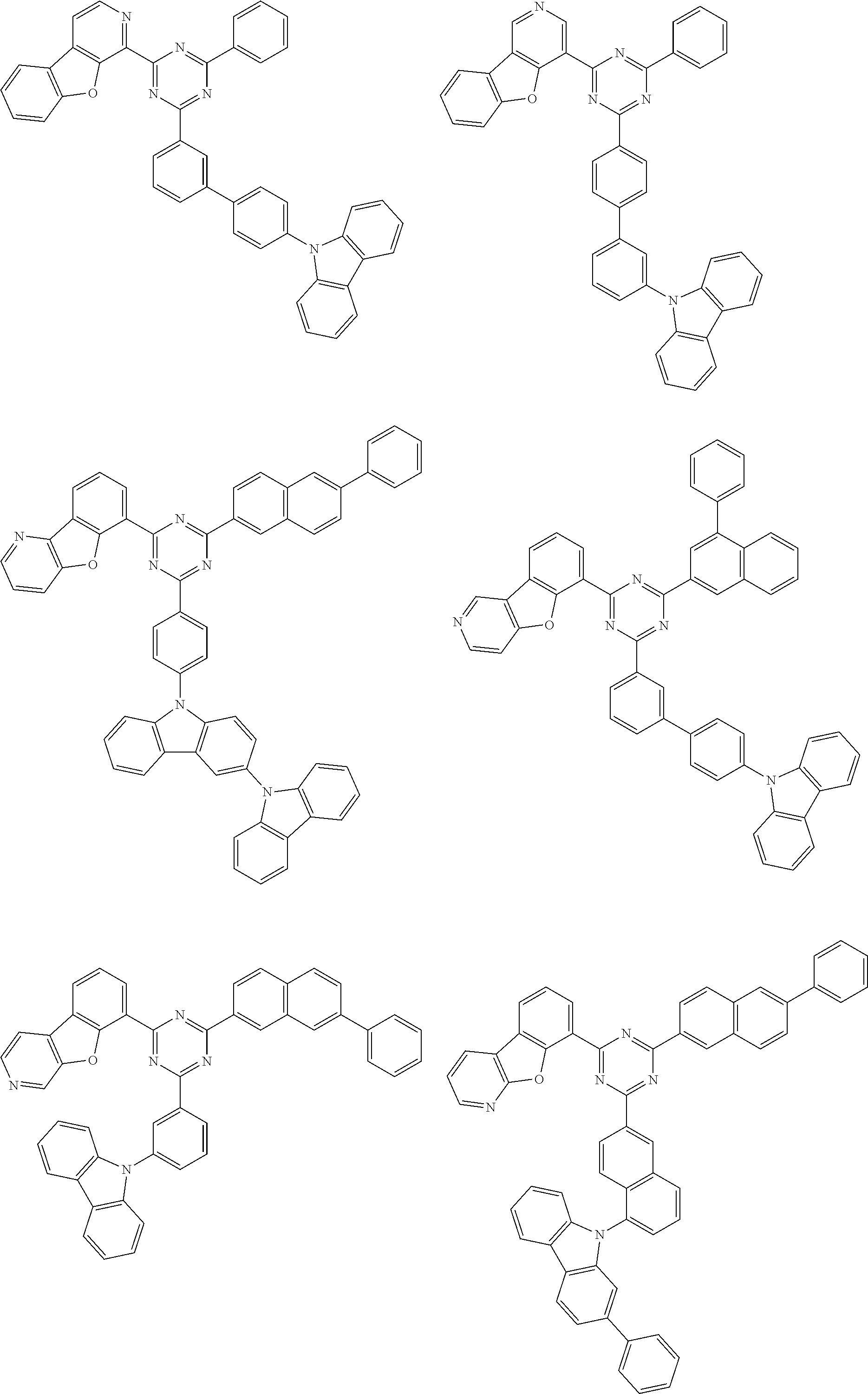

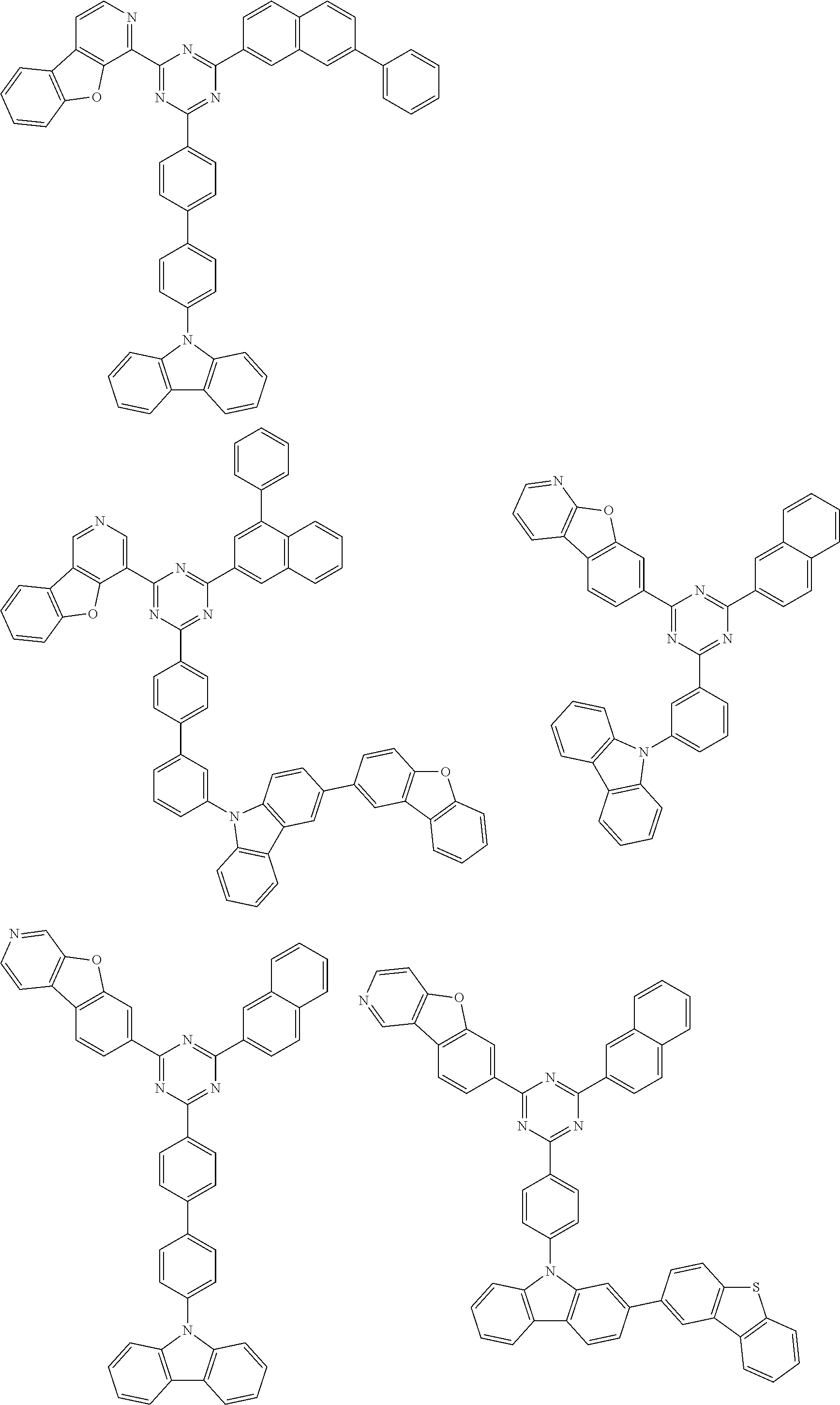

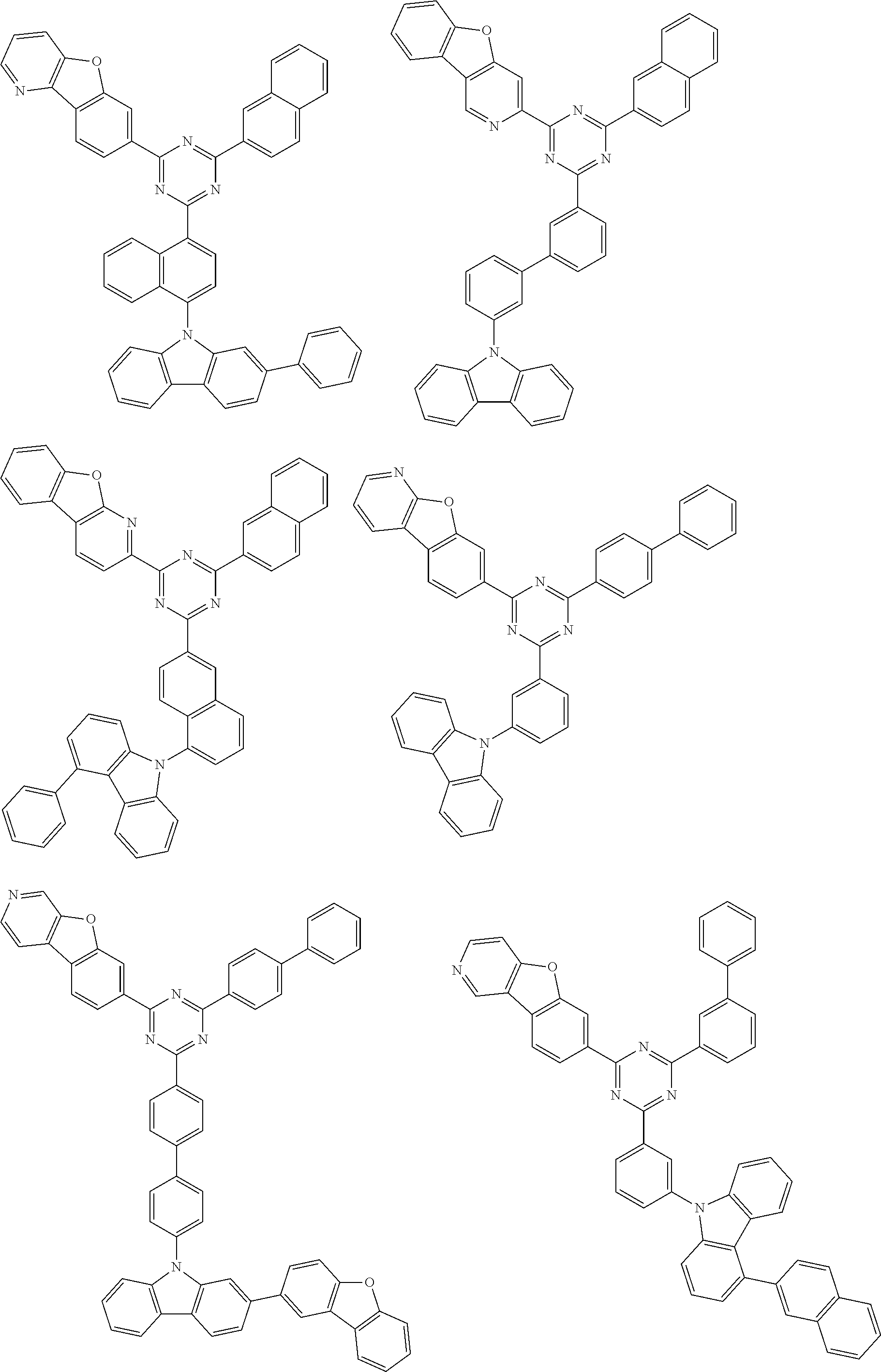

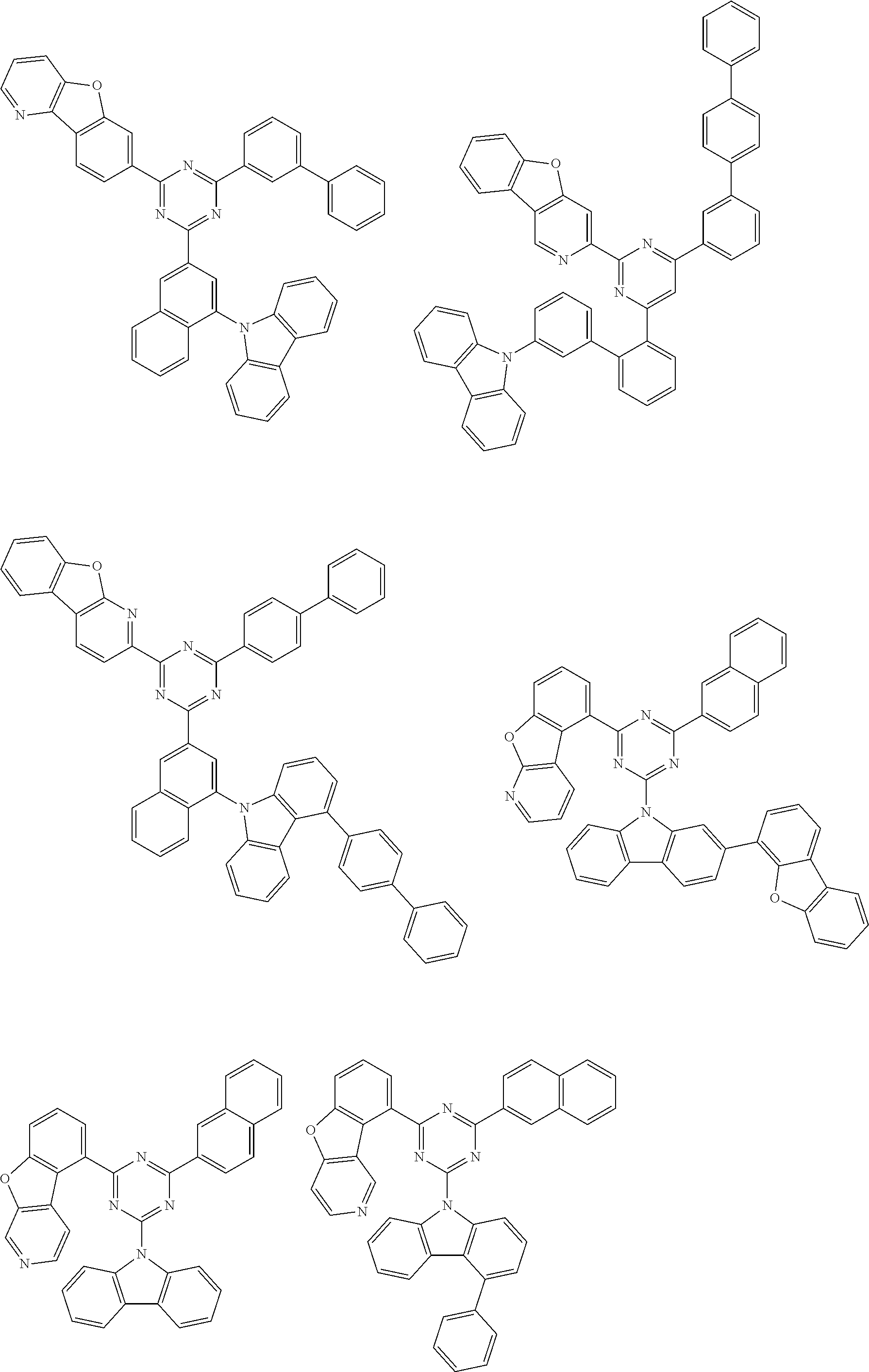

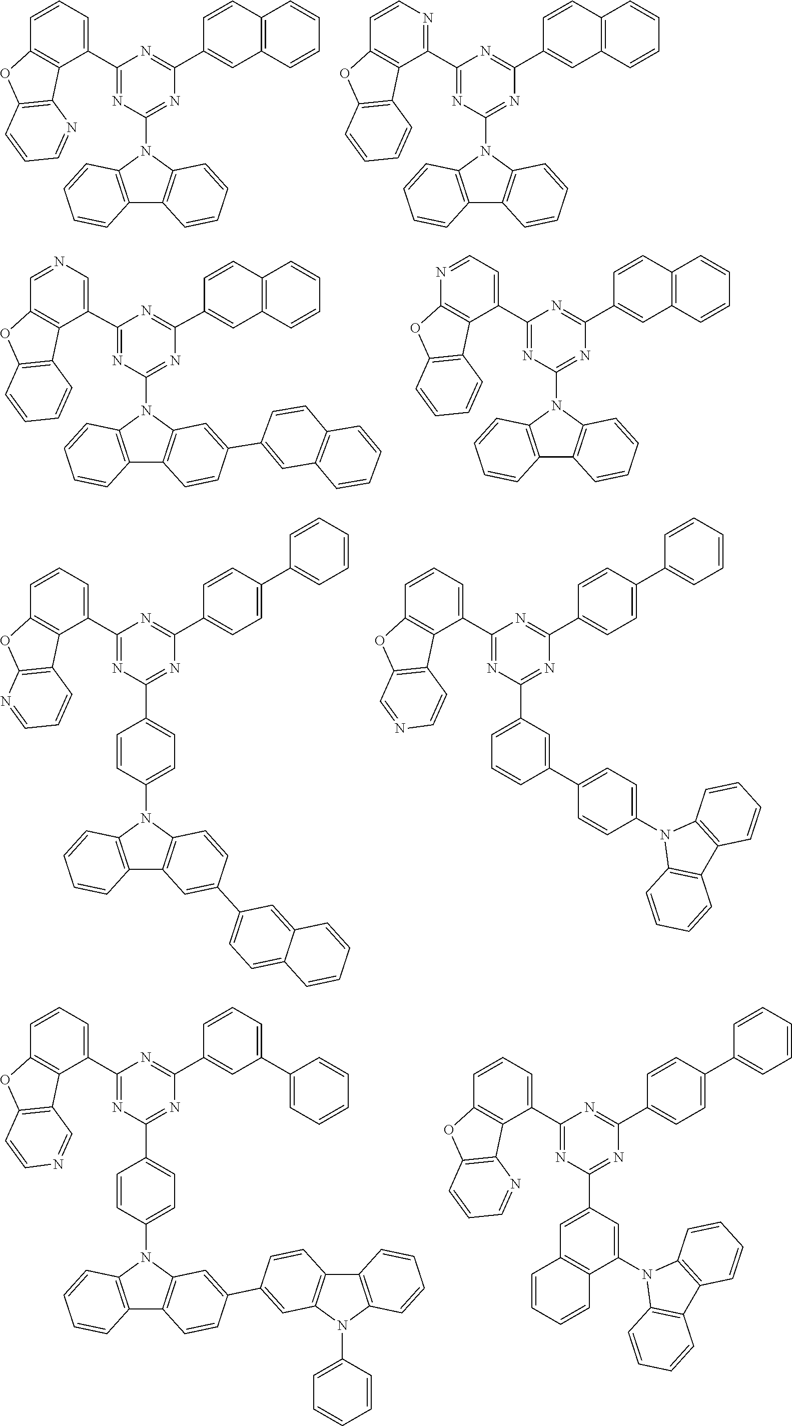

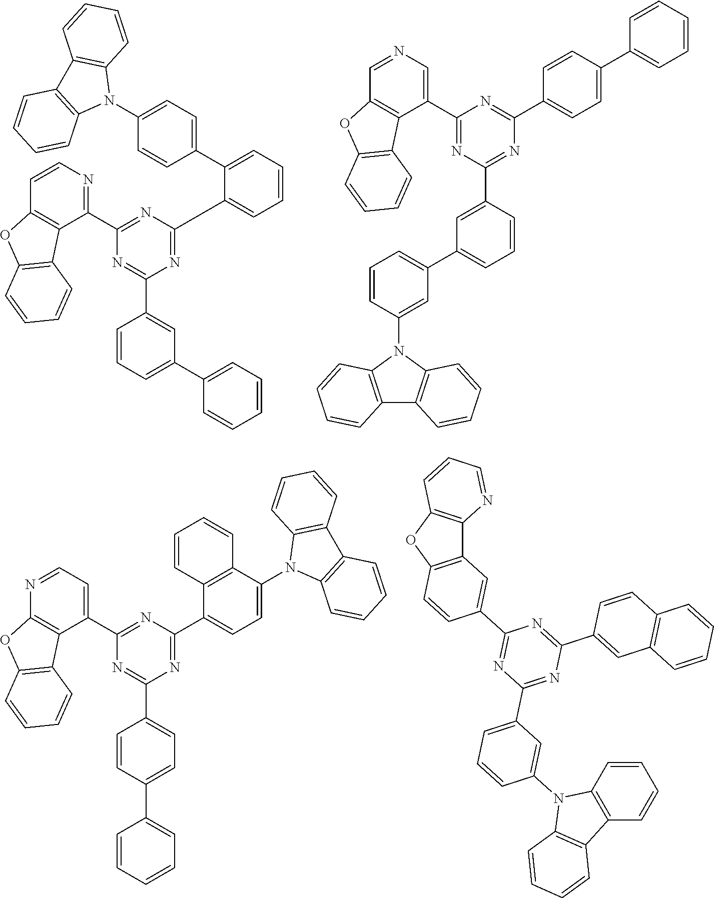

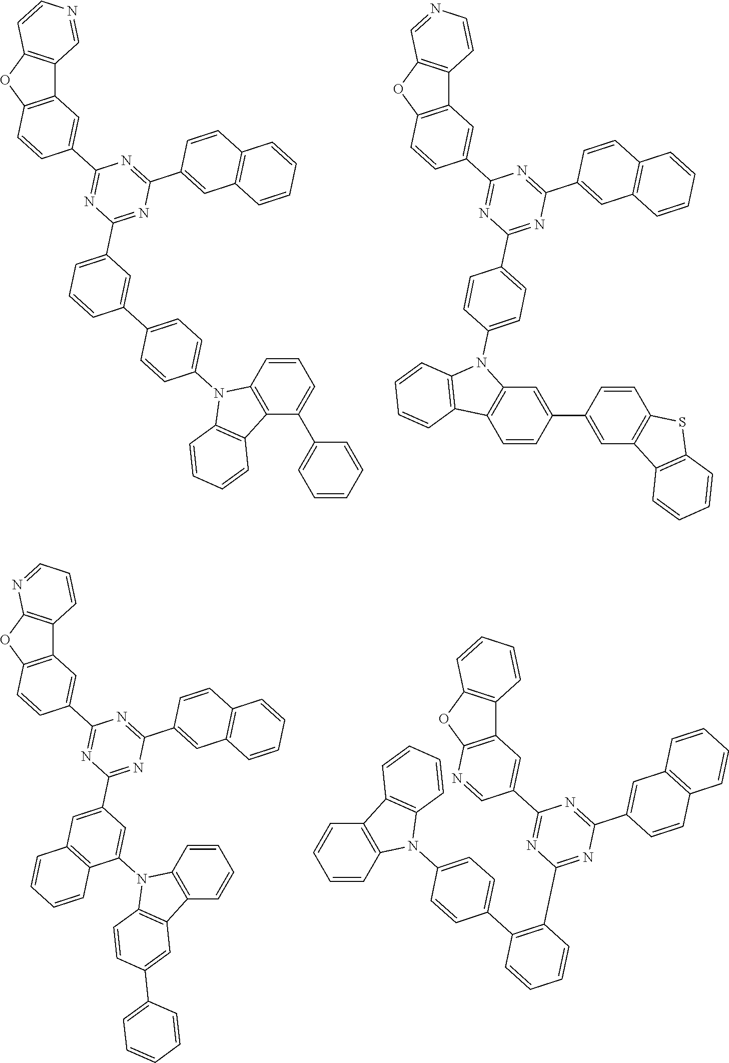 [SMILES: c1ccc(-c2cccc3c2c2ccccc2n3-c2ccc(-c3cccc(-c4nc(-c5ccc6ccccc6c5)nc(-c5ccc6oc7ccncc7c6c5)n4)c3)cc2)cc1 ; c1ccc2cc(-c3nc(-c4ccc(-n5c6ccccc6c6ccc(-c7ccc8sc9ccccc9c8c7)cc65)cc4)nc(-c4ccc5oc6cnccc6c5c4)n3)ccc2c1 ; c1ccc(-c2ccc3c(c2)c2ccccc2n3-c2cc(-c3nc(-c4ccc5ccccc5c4)nc(-c4ccc5oc6ncccc6c5c4)n3)cc3ccccc23)cc1 ; c1ccc(-c2nc(-c3ccc4ccccc4c3)nc(-c3cnc4oc5ccccc5c4c3)n2)c(-c2ccc(-n3c4ccccc4c4ccccc43)cc2)c1]

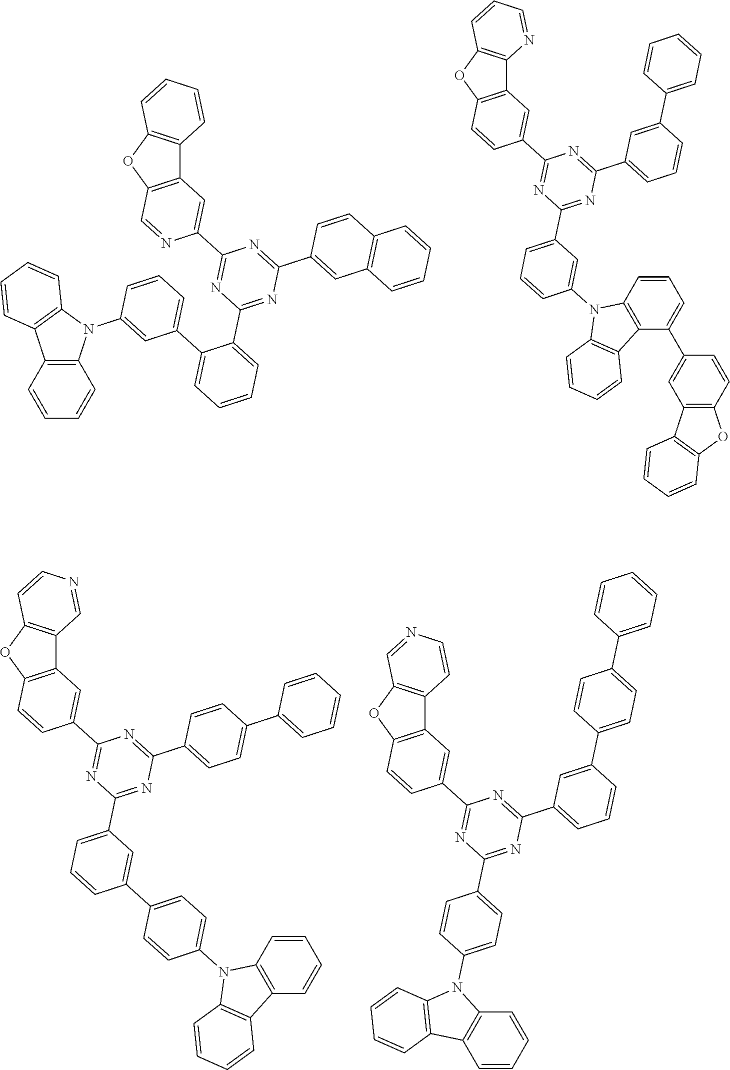

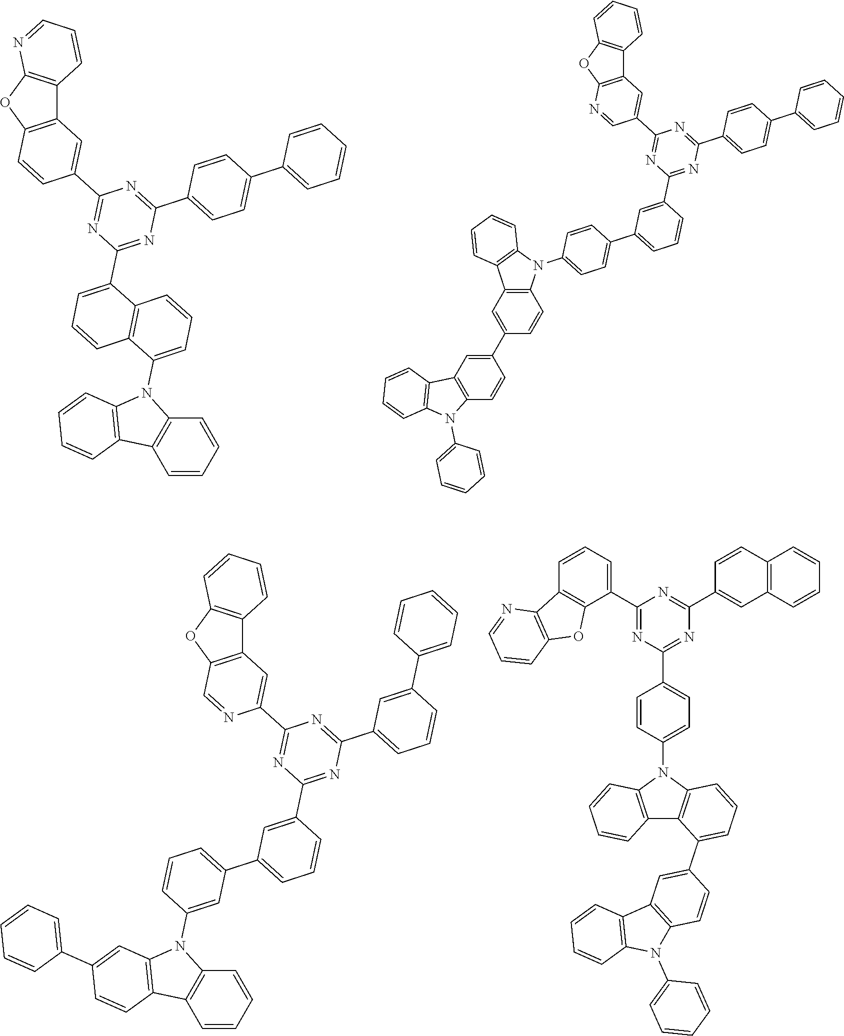

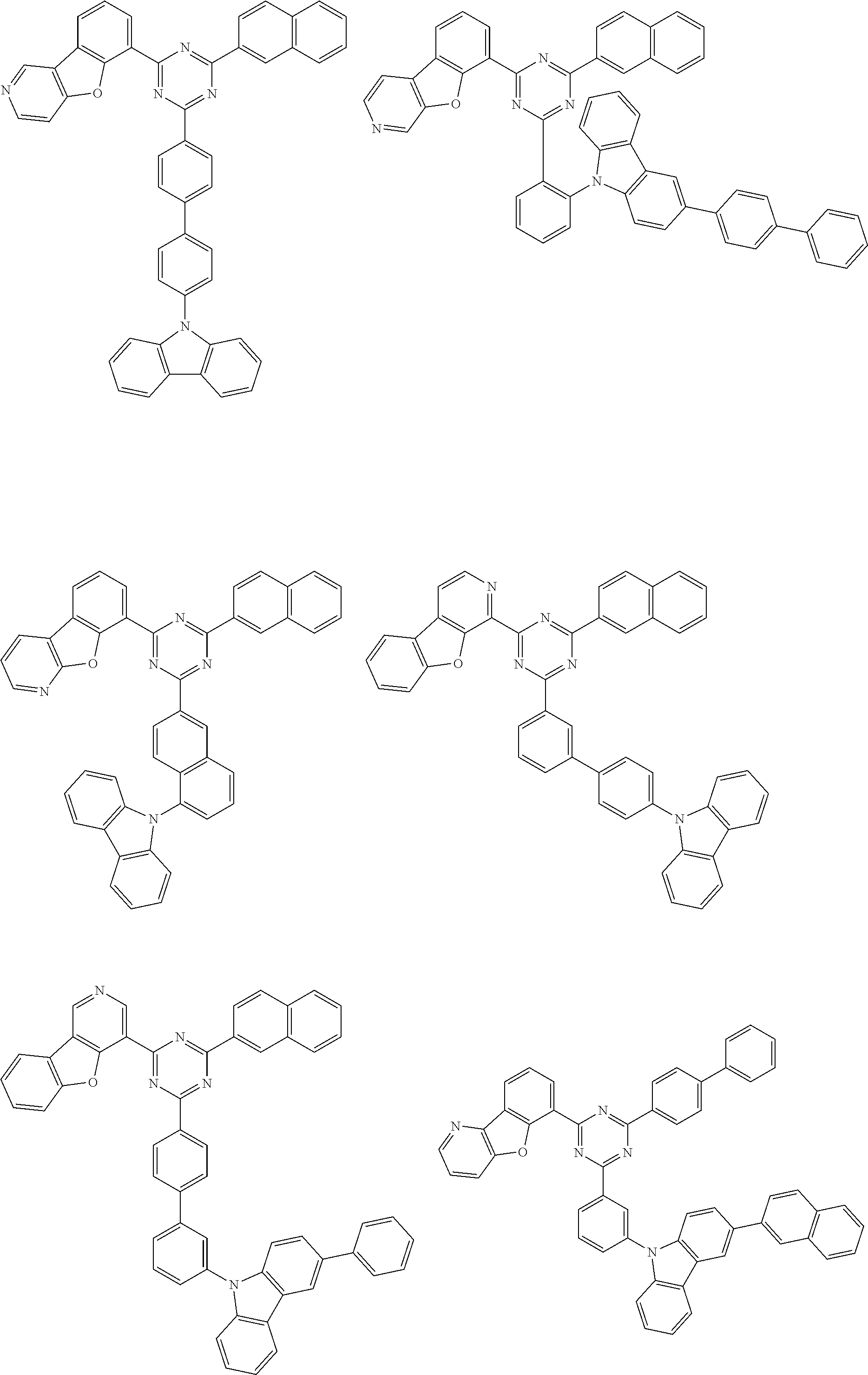

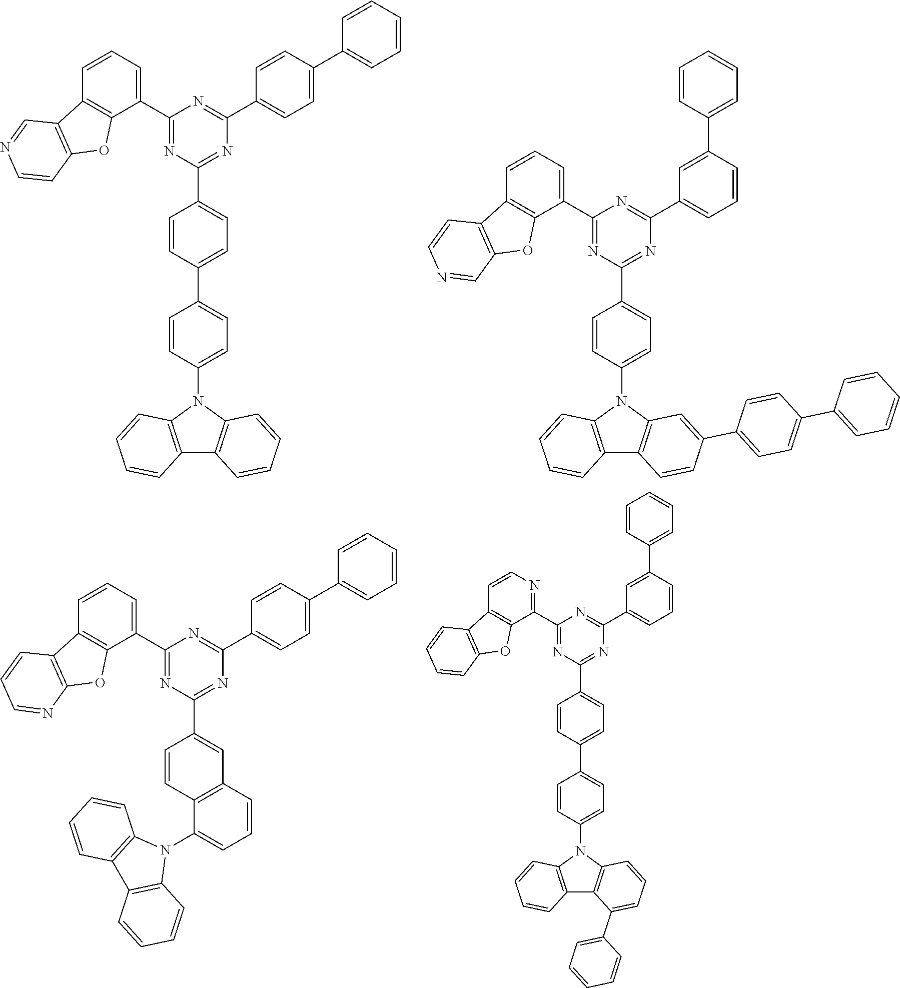

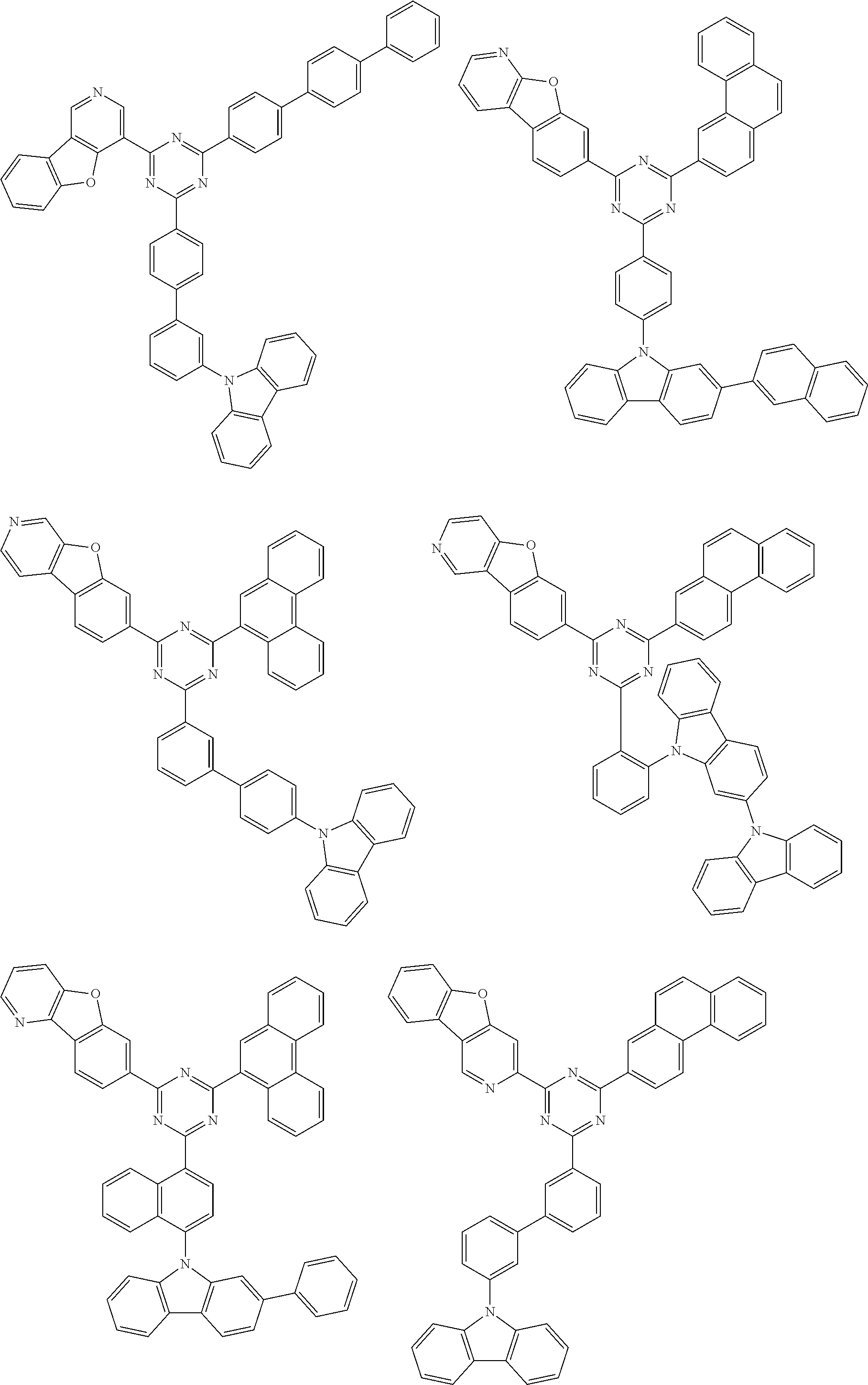

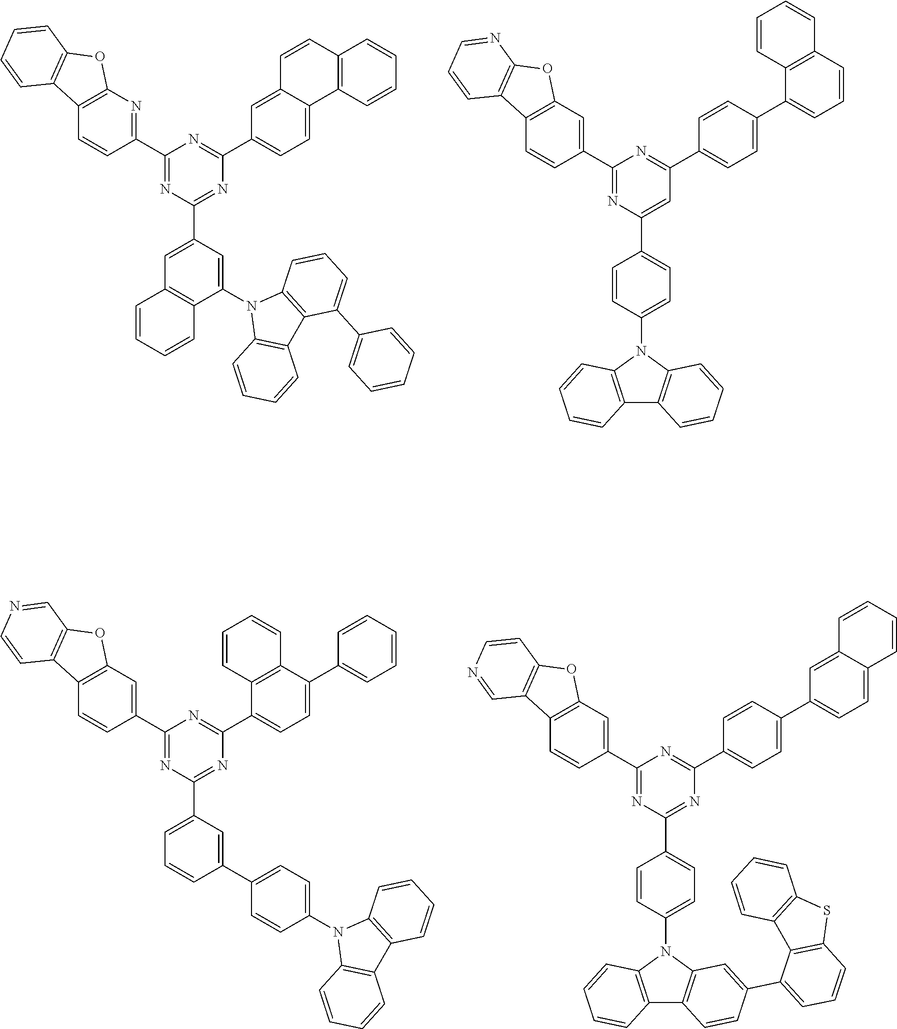 [SMILES: c1ccc(-c2cccc3c2c2ccccc2n3-c2cc(-c3nc(-c4ccc5c(ccc6ccccc65)c4)nc(-c4ccc5c(n4)oc4ccccc45)n3)cc3ccccc23)cc1 ; c1ccc2c(-c3ccc(-c4cc(-c5ccc(-n6c7ccccc7c7ccccc76)cc5)nc(-c5ccc6c(c5)oc5ncccc56)n4)cc3)cccc2c1 ; c1ccc(-c2ccc(-c3nc(-c4cccc(-c5ccc(-n6c7ccccc7c7ccccc76)cc5)c4)nc(-c4ccc5c(c4)oc4cnccc45)n3)c3ccccc23)cc1 ; c1ccc2cc(-c3ccc(-c4nc(-c5ccc(-n6c7ccccc7c7ccc(-c8cccc9sc%10ccccc%10c89)cc76)cc5)nc(-c5ccc6c(c5)oc5ccncc56)n4)cc3)ccc2c1]

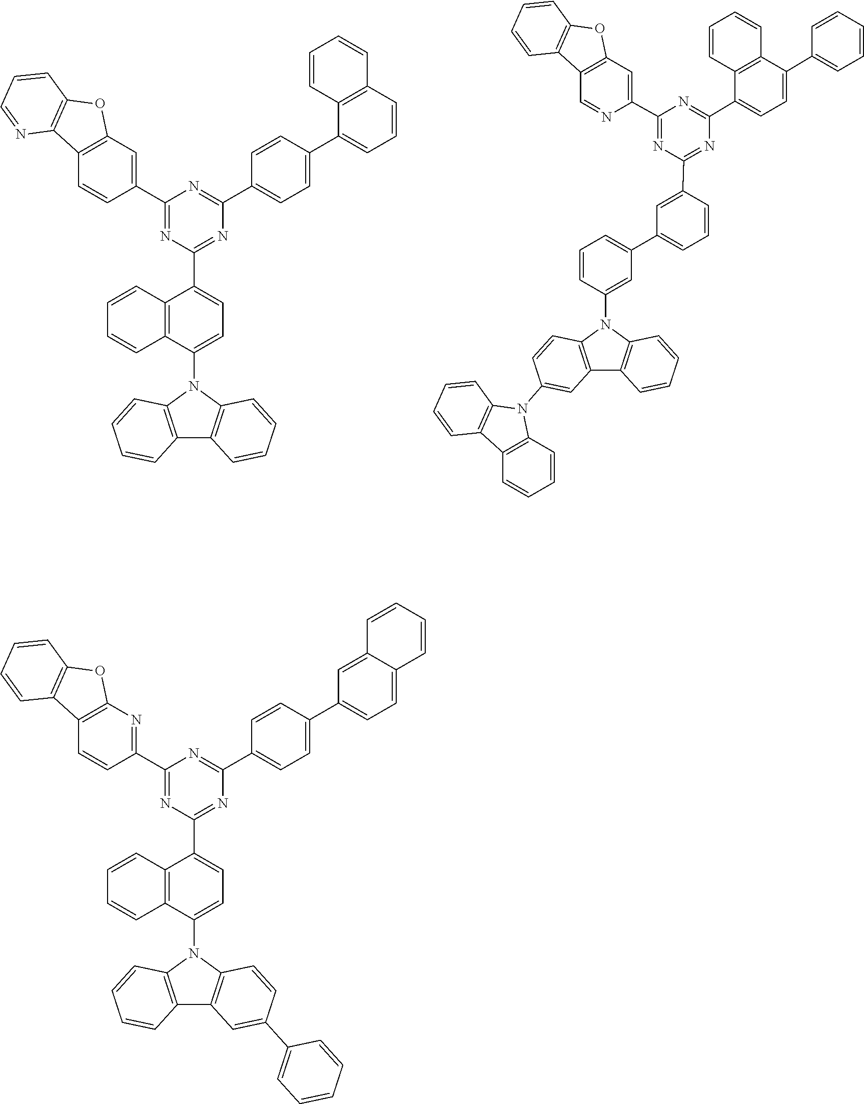 [SMILES: c1ccc2c(-c3ccc(-c4nc(-c5ccc6c(c5)oc5cccnc56)nc(-c5ccc(-n6c7ccccc7c7ccccc76)c6ccccc56)n4)cc3)cccc2c1 ; c1ccc(-c2ccc(-c3nc(-c4cccc(-c5cccc(-n6c7ccccc7c7cc(-n8c9ccccc9c9ccccc98)ccc76)c5)c4)nc(-c4cc5oc6ccccc6c5cn4)n3)c3ccccc23)cc1 ; c1ccc(-c2ccc3c(c2)c2ccccc2n3-c2ccc(-c3nc(-c4ccc(-c5ccc6ccccc6c5)cc4)nc(-c4ccc5c(n4)oc4ccccc45)n3)c3ccccc23)cc1]

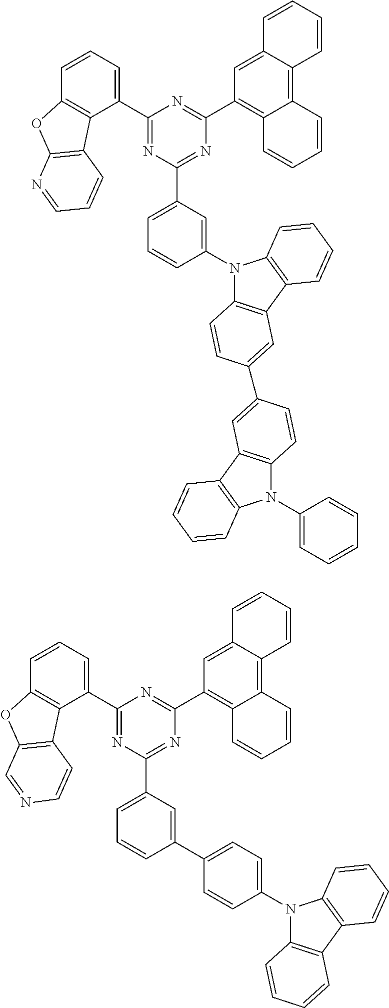 [SMILES: c1ccc(-n2c3ccccc3c3cc(-c4ccc5c(c4)c4ccccc4n5-c4cccc(-c5nc(-c6cc7ccccc7c7ccccc67)nc(-c6cccc7oc8ncccc8c67)n5)c4)ccc32)cc1 ; c1cc(-c2ccc(-n3c4ccccc4c4ccccc43)cc2)cc(-c2nc(-c3cc4ccccc4c4ccccc34)nc(-c3cccc4oc5cnccc5c34)n2)c1]

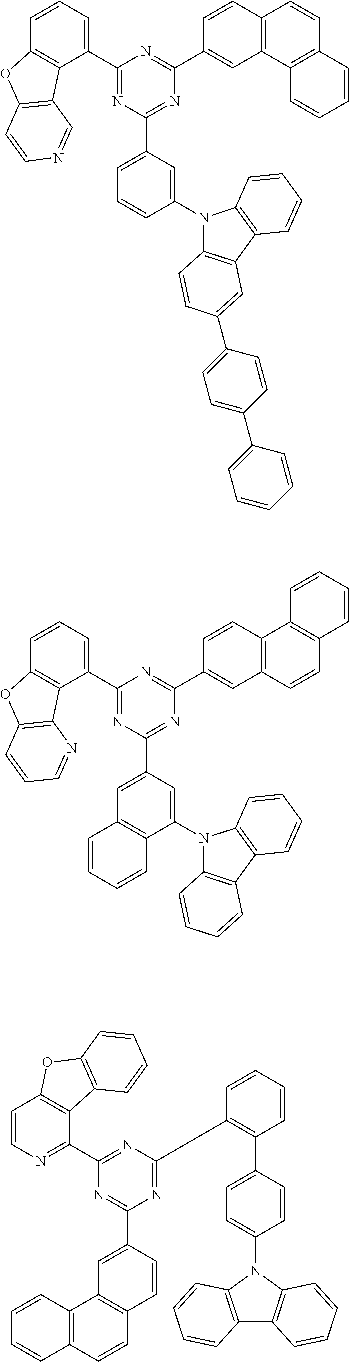 [SMILES: c1ccc(-c2ccc(-c3ccc4c(c3)c3ccccc3n4-c3cccc(-c4nc(-c5ccc6ccc7ccccc7c6c5)nc(-c5cccc6oc7ccncc7c56)n4)c3)cc2)cc1 ; c1ccc2c(-n3c4ccccc4c4ccccc43)cc(-c3nc(-c4ccc5c(ccc6ccccc65)c4)nc(-c4cccc5oc6cccnc6c45)n3)cc2c1 ; c1ccc(-c2nc(-c3ccc4ccc5ccccc5c4c3)nc(-c3nccc4oc5ccccc5c34)n2)c(-c2ccc(-n3c4ccccc4c4ccccc43)cc2)c1]

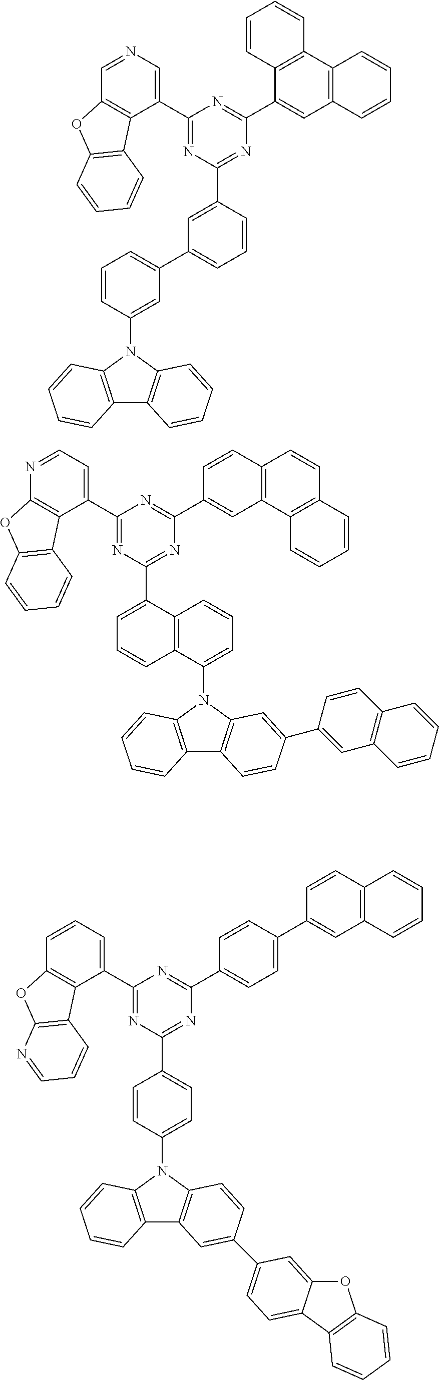

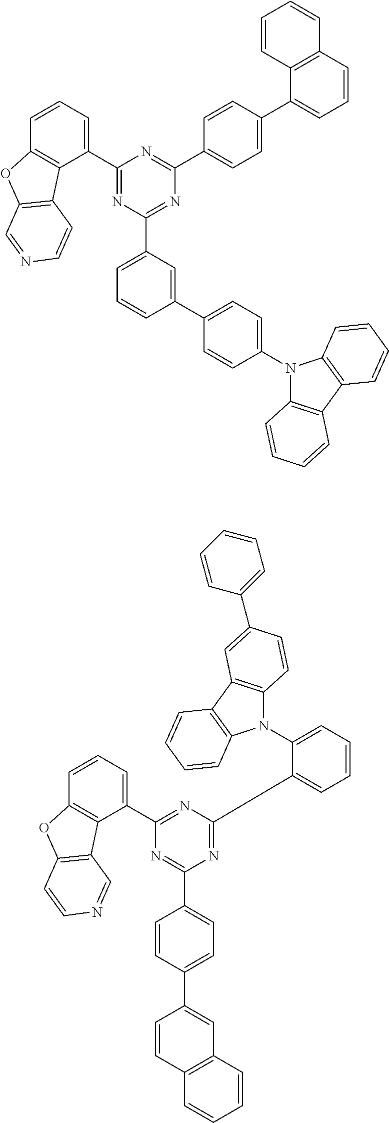 [SMILES: c1cc(-c2ccc(-n3c4ccccc4c4ccccc43)cc2)cc(-c2nc(-c3ccc(-c4cccc5ccccc45)cc3)nc(-c3cccc4oc5cnccc5c34)n2)c1 ; c1ccc(-c2ccc3c(c2)c2ccccc2n3-c2ccccc2-c2nc(-c3ccc(-c4ccc5ccccc5c4)cc3)nc(-c3cccc4oc5ccncc5c34)n2)cc1]

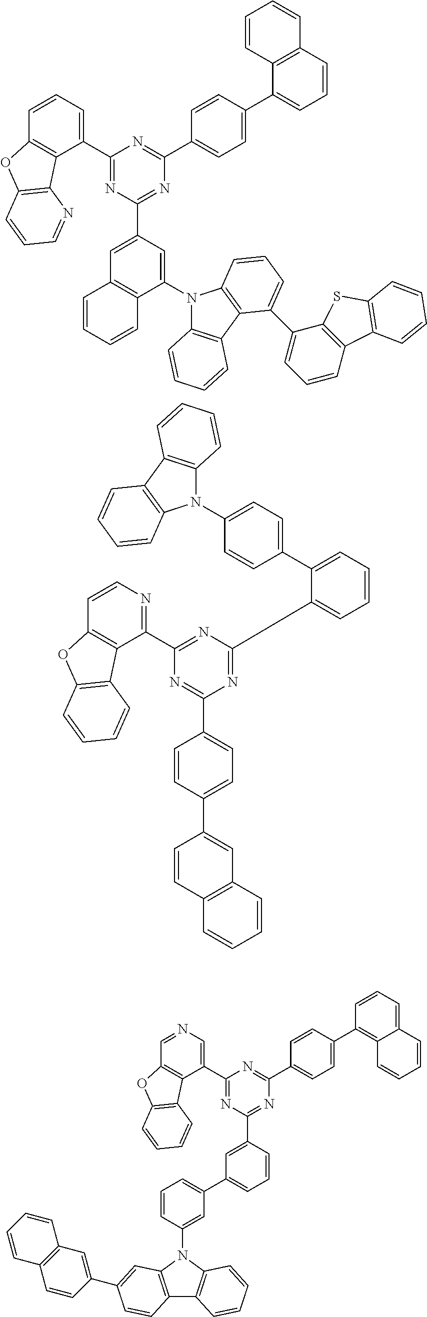

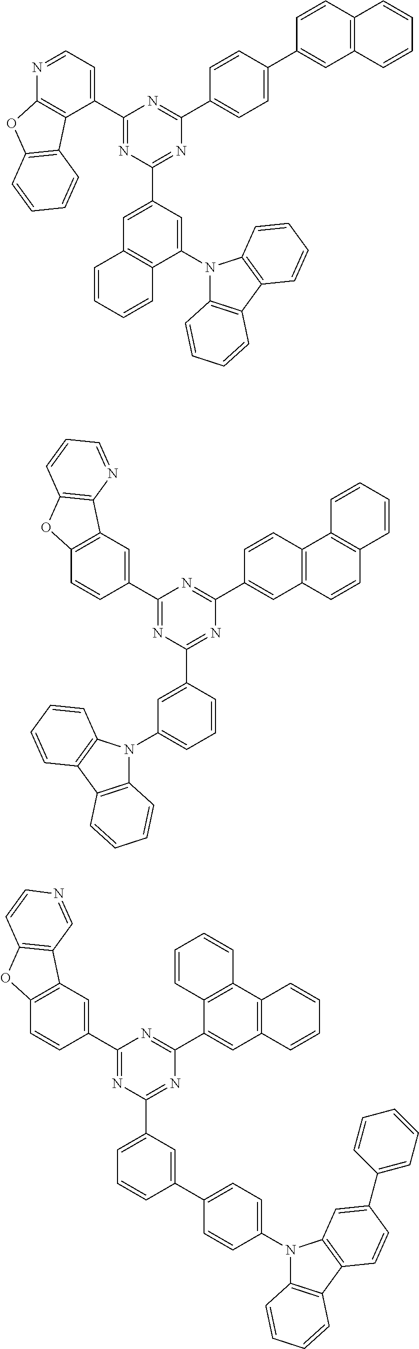 [SMILES: c1ccc2cc(-c3ccc(-c4nc(-c5cc(-n6c7ccccc7c7ccccc76)c6ccccc6c5)nc(-c5ccnc6oc7ccccc7c56)n4)cc3)ccc2c1 ; c1cc(-c2nc(-c3ccc4c(ccc5ccccc54)c3)nc(-c3ccc4oc5cccnc5c4c3)n2)cc(-n2c3ccccc3c3ccccc32)c1 ; c1ccc(-c2ccc3c4ccccc4n(-c4ccc(-c5cccc(-c6nc(-c7ccc8oc9ccncc9c8c7)nc(-c7cc8ccccc8c8ccccc78)n6)c5)cc4)c3c2)cc1]

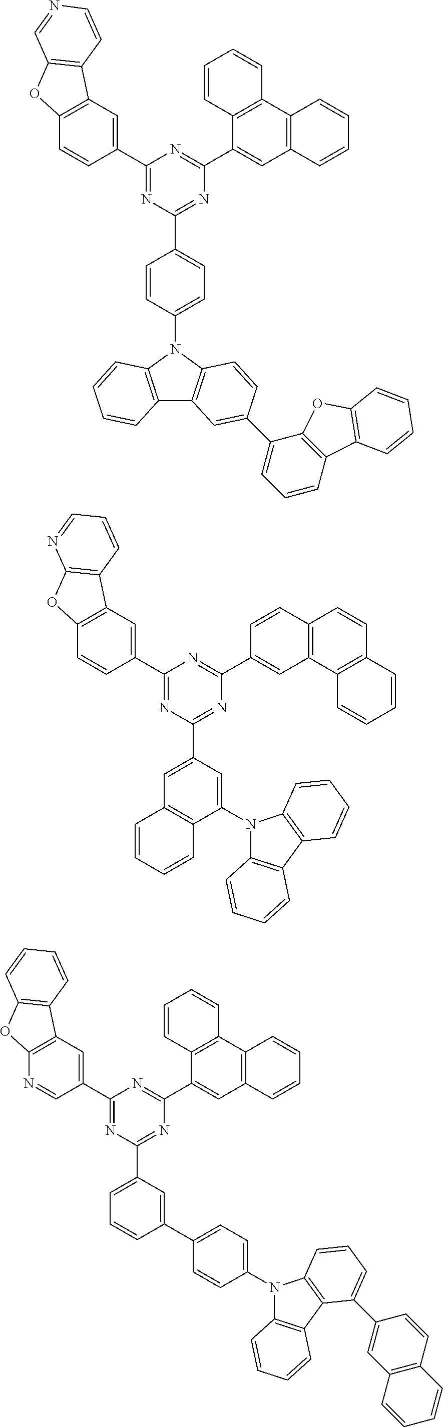 [SMILES: c1ccc2c(c1)cc(-c1nc(-c3ccc(-n4c5ccccc5c5cc(-c6cccc7c6oc6ccccc67)ccc54)cc3)nc(-c3ccc4oc5cnccc5c4c3)n1)c1ccccc12 ; c1ccc2c(-n3c4ccccc4c4ccccc43)cc(-c3nc(-c4ccc5ccc6ccccc6c5c4)nc(-c4ccc5oc6ncccc6c5c4)n3)cc2c1 ; c1cc(-c2ccc(-n3c4ccccc4c4c(-c5ccc6ccccc6c5)cccc43)cc2)cc(-c2nc(-c3cnc4oc5ccccc5c4c3)nc(-c3cc4ccccc4c4ccccc34)n2)c1]

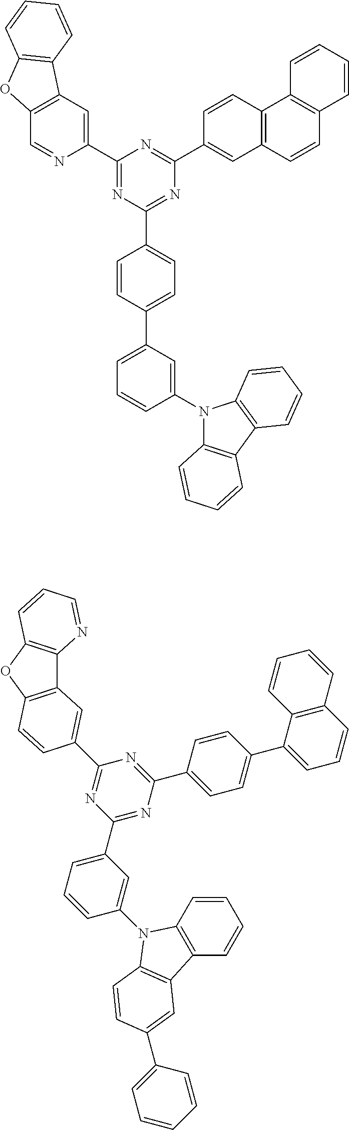 [SMILES: c1cc(-c2ccc(-c3nc(-c4ccc5c(ccc6ccccc65)c4)nc(-c4cc5c(cn4)oc4ccccc45)n3)cc2)cc(-n2c3ccccc3c3ccccc32)c1 ; c1ccc(-c2ccc3c(c2)c2ccccc2n3-c2cccc(-c3nc(-c4ccc(-c5cccc6ccccc56)cc4)nc(-c4ccc5oc6cccnc6c5c4)n3)c2)cc1]

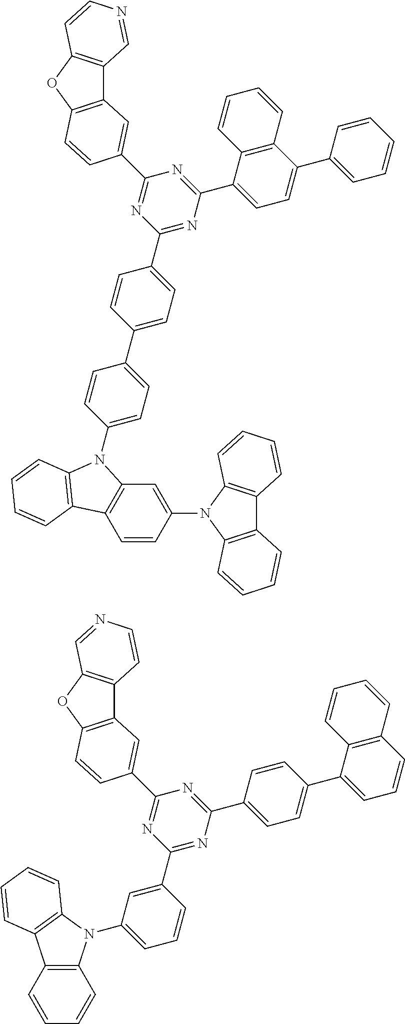 [SMILES: c1ccc(-c2ccc(-c3nc(-c4ccc(-c5ccc(-n6c7ccccc7c7ccc(-n8c9ccccc9c9ccccc98)cc76)cc5)cc4)nc(-c4ccc5oc6ccncc6c5c4)n3)c3ccccc23)cc1 ; c1cc(-c2nc(-c3ccc(-c4cccc5ccccc45)cc3)nc(-c3ccc4oc5cnccc5c4c3)n2)cc(-n2c3ccccc3c3ccccc32)c1]

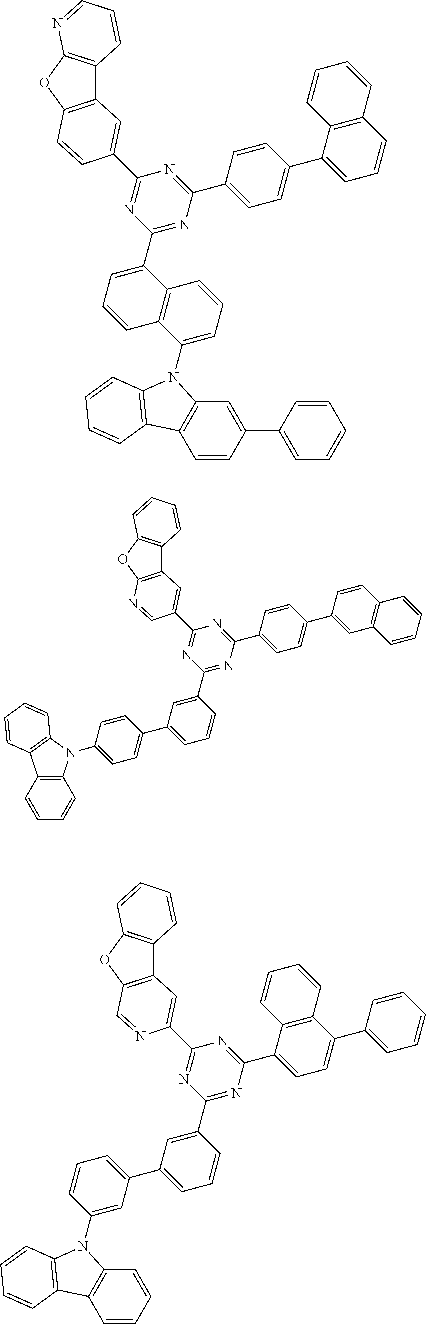 [SMILES: c1ccc(-c2ccc3c4ccccc4n(-c4cccc5c(-c6nc(-c7ccc(-c8cccc9ccccc89)cc7)nc(-c7ccc8oc9ncccc9c8c7)n6)cccc45)c3c2)cc1 ; c1cc(-c2ccc(-n3c4ccccc4c4ccccc43)cc2)cc(-c2nc(-c3ccc(-c4ccc5ccccc5c4)cc3)nc(-c3cnc4oc5ccccc5c4c3)n2)c1 ; c1ccc(-c2ccc(-c3nc(-c4cccc(-c5cccc(-n6c7ccccc7c7ccccc76)c5)c4)nc(-c4cc5c(cn4)oc4ccccc45)n3)c3ccccc23)cc1]

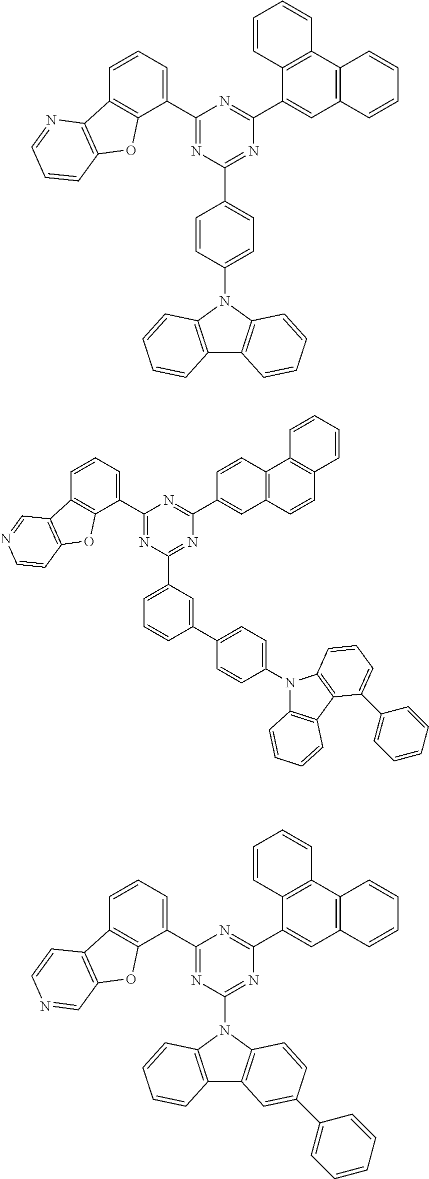 [SMILES: c1ccc2c(c1)cc(-c1nc(-c3ccc(-n4c5ccccc5c5ccccc54)cc3)nc(-c3cccc4c3oc3cccnc34)n1)c1ccccc12 ; c1ccc(-c2cccc3c2c2ccccc2n3-c2ccc(-c3cccc(-c4nc(-c5ccc6c(ccc7ccccc76)c5)nc(-c5cccc6c5oc5ccncc56)n4)c3)cc2)cc1 ; c1ccc(-c2ccc3c(c2)c2ccccc2n3-c2nc(-c3cc4ccccc4c4ccccc34)nc(-c3cccc4c3oc3cnccc34)n2)cc1]

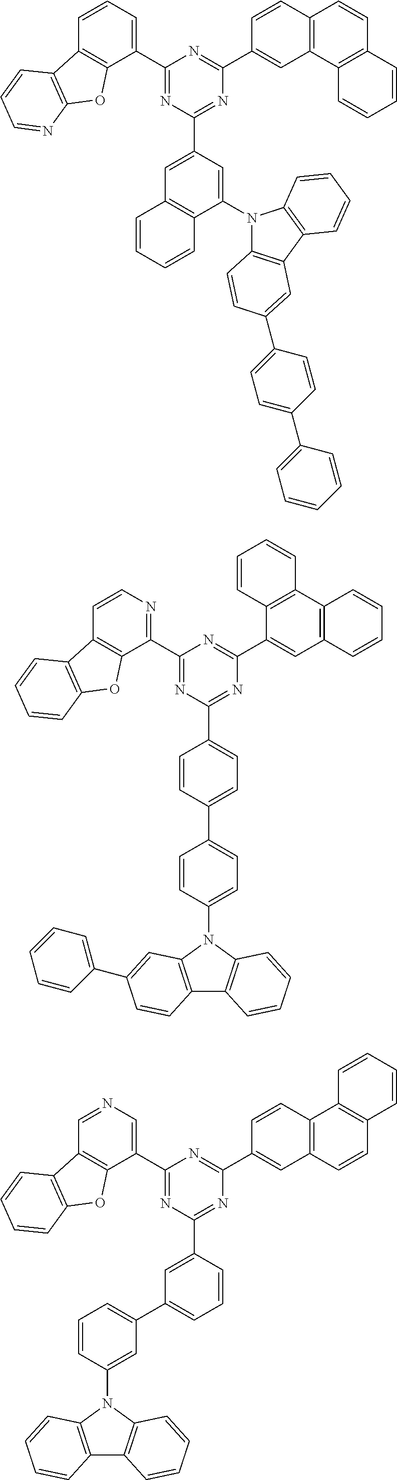

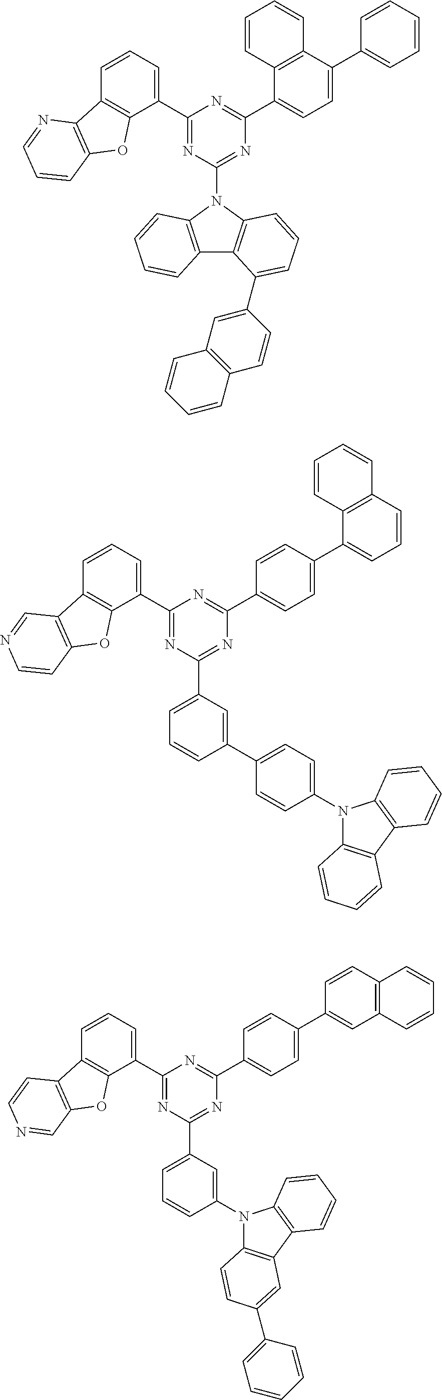 [SMILES: c1ccc(-c2ccc(-c3nc(-c4cccc5c4oc4cccnc45)nc(-n4c5ccccc5c5c(-c6ccc7ccccc7c6)cccc54)n3)c3ccccc23)cc1 ; c1cc(-c2ccc(-n3c4ccccc4c4ccccc43)cc2)cc(-c2nc(-c3ccc(-c4cccc5ccccc45)cc3)nc(-c3cccc4c3oc3ccncc34)n2)c1 ; c1ccc(-c2ccc3c(c2)c2ccccc2n3-c2cccc(-c3nc(-c4ccc(-c5ccc6ccccc6c5)cc4)nc(-c4cccc5c4oc4cnccc45)n3)c2)cc1]

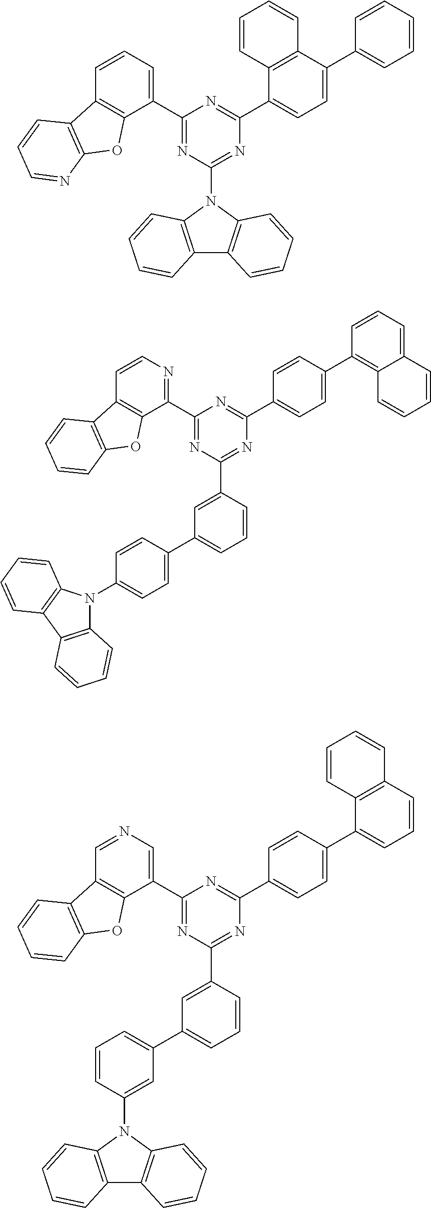 [SMILES: c1ccc(-c2ccc(-c3nc(-c4cccc5c4oc4ncccc45)nc(-n4c5ccccc5c5ccccc54)n3)c3ccccc23)cc1 ; c1cc(-c2ccc(-n3c4ccccc4c4ccccc43)cc2)cc(-c2nc(-c3ccc(-c4cccc5ccccc45)cc3)nc(-c3nccc4c3oc3ccccc34)n2)c1 ; c1cc(-c2cccc(-n3c4ccccc4c4ccccc43)c2)cc(-c2nc(-c3ccc(-c4cccc5ccccc45)cc3)nc(-c3cncc4c3oc3ccccc34)n2)c1]

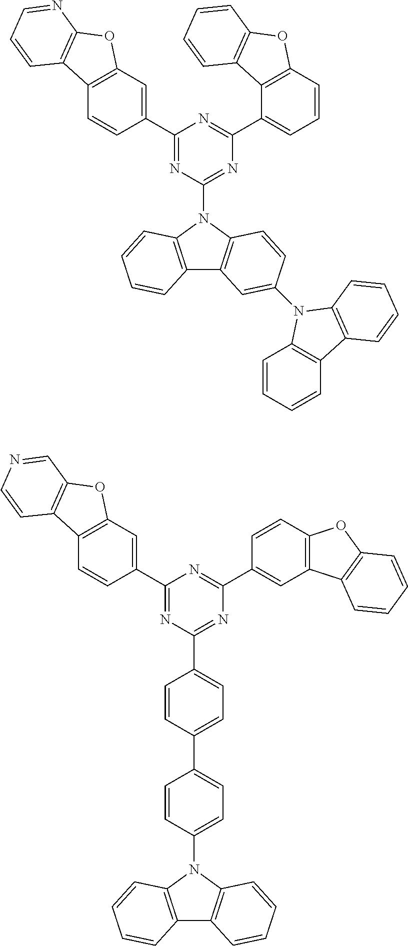 [SMILES: c1ccc2c(c1)oc1cccc(-c3nc(-c4ccc5c(c4)oc4ncccc45)nc(-n4c5ccccc5c5cc(-n6c7ccccc7c7ccccc76)ccc54)n3)c12 ; c1ccc2c(c1)oc1ccc(-c3nc(-c4ccc(-c5ccc(-n6c7ccccc7c7ccccc76)cc5)cc4)nc(-c4ccc5c(c4)oc4cnccc45)n3)cc12]

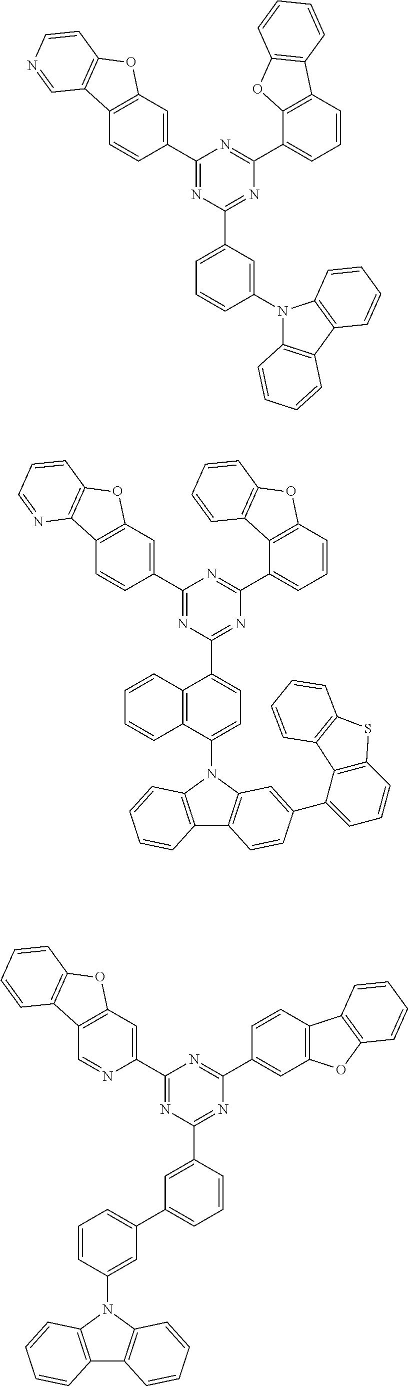 [SMILES: c1cc(-c2nc(-c3ccc4c(c3)oc3ccncc34)nc(-c3cccc4c3oc3ccccc34)n2)cc(-n2c3ccccc3c3ccccc32)c1 ; c1ccc2c(c1)oc1cccc(-c3nc(-c4ccc5c(c4)oc4cccnc45)nc(-c4ccc(-n5c6ccccc6c6ccc(-c7cccc8sc9ccccc9c78)cc65)c5ccccc45)n3)c12 ; c1cc(-c2cccc(-n3c4ccccc4c4ccccc43)c2)cc(-c2nc(-c3ccc4c(c3)oc3ccccc34)nc(-c3cc4oc5ccccc5c4cn3)n2)c1]

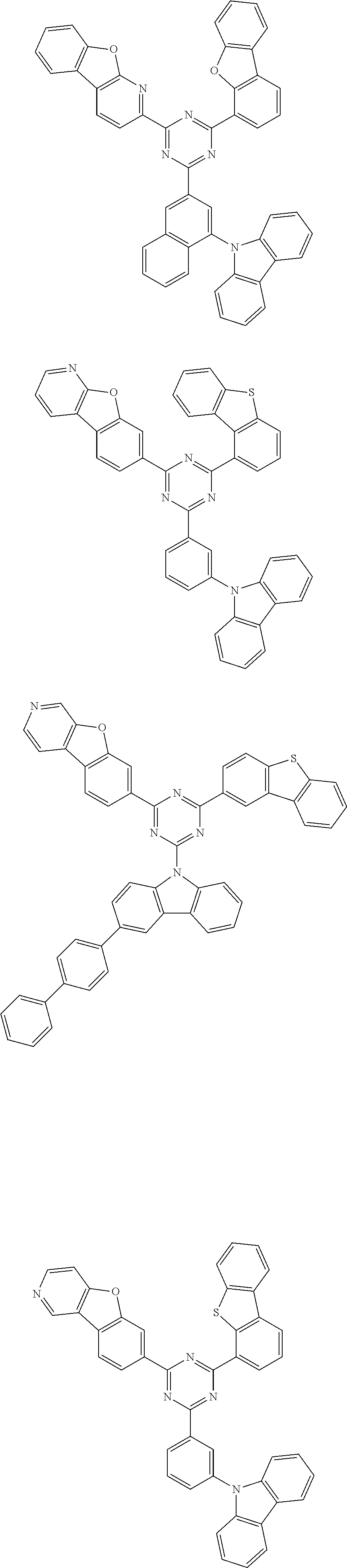 [SMILES: c1ccc2c(-n3c4ccccc4c4ccccc43)cc(-c3nc(-c4ccc5c(n4)oc4ccccc45)nc(-c4cccc5c4oc4ccccc45)n3)cc2c1 ; c1cc(-c2nc(-c3ccc4c(c3)oc3ncccc34)nc(-c3cccc4sc5ccccc5c34)n2)cc(-n2c3ccccc3c3ccccc32)c1 ; c1ccc(-c2ccc(-c3ccc4c(c3)c3ccccc3n4-c3nc(-c4ccc5c(c4)oc4cnccc45)nc(-c4ccc5sc6ccccc6c5c4)n3)cc2)cc1 ; c1cc(-c2nc(-c3ccc4c(c3)oc3ccncc34)nc(-c3cccc4c3sc3ccccc34)n2)cc(-n2c3ccccc3c3ccccc32)c1]

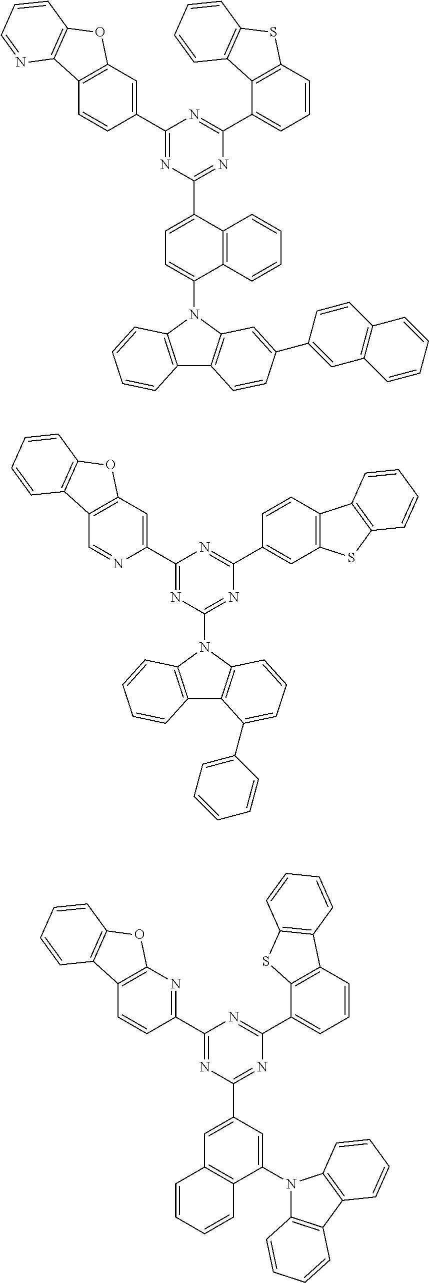 [SMILES: c1ccc2cc(-c3ccc4c5ccccc5n(-c5ccc(-c6nc(-c7ccc8c(c7)oc7cccnc78)nc(-c7cccc8sc9ccccc9c78)n6)c6ccccc56)c4c3)ccc2c1 ; c1ccc(-c2cccc3c2c2ccccc2n3-c2nc(-c3ccc4c(c3)sc3ccccc34)nc(-c3cc4oc5ccccc5c4cn3)n2)cc1 ; c1ccc2c(-n3c4ccccc4c4ccccc43)cc(-c3nc(-c4ccc5c(n4)oc4ccccc45)nc(-c4cccc5c4sc4ccccc45)n3)cc2c1]

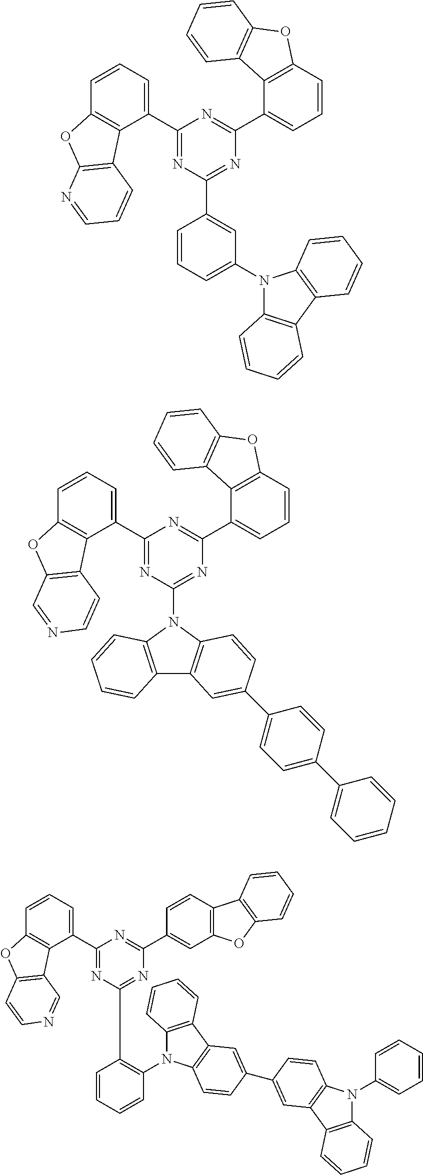 [SMILES: c1cc(-c2nc(-c3cccc4oc5ccccc5c34)nc(-c3cccc4oc5ncccc5c34)n2)cc(-n2c3ccccc3c3ccccc32)c1 ; c1ccc(-c2ccc(-c3ccc4c(c3)c3ccccc3n4-c3nc(-c4cccc5oc6ccccc6c45)nc(-c4cccc5oc6cnccc6c45)n3)cc2)cc1 ; c1ccc(-n2c3ccccc3c3cc(-c4ccc5c(c4)c4ccccc4n5-c4ccccc4-c4nc(-c5ccc6c(c5)oc5ccccc56)nc(-c5cccc6oc7ccncc7c56)n4)ccc32)cc1]

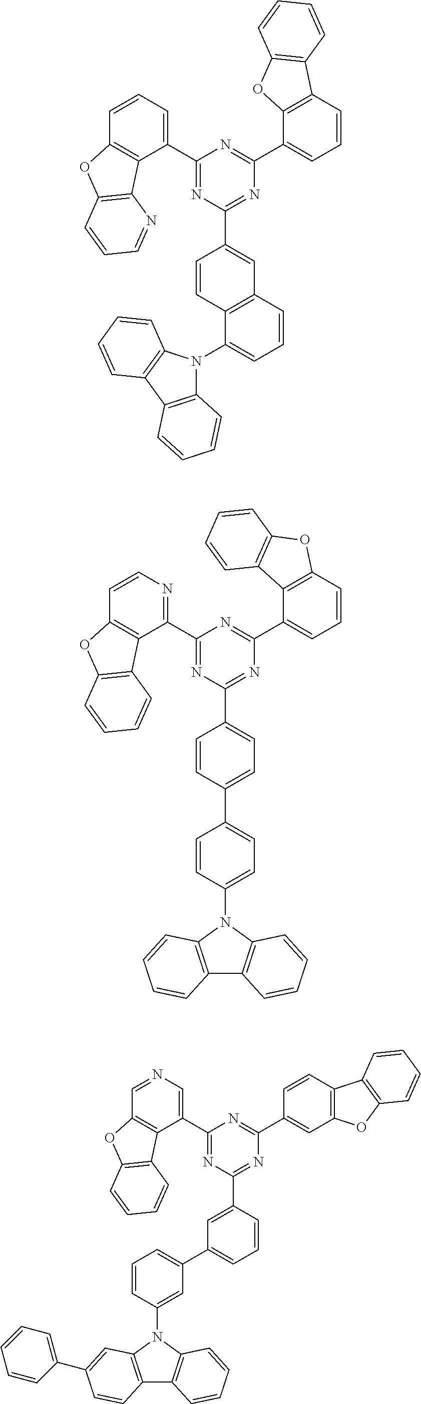 [SMILES: c1cc(-n2c3ccccc3c3ccccc32)c2ccc(-c3nc(-c4cccc5c4oc4ccccc45)nc(-c4cccc5oc6cccnc6c45)n3)cc2c1 ; c1ccc2c(c1)oc1cccc(-c3nc(-c4ccc(-c5ccc(-n6c7ccccc7c7ccccc76)cc5)cc4)nc(-c4nccc5oc6ccccc6c45)n3)c12 ; c1ccc(-c2ccc3c4ccccc4n(-c4cccc(-c5cccc(-c6nc(-c7ccc8c(c7)oc7ccccc78)nc(-c7cncc8oc9ccccc9c78)n6)c5)c4)c3c2)cc1]

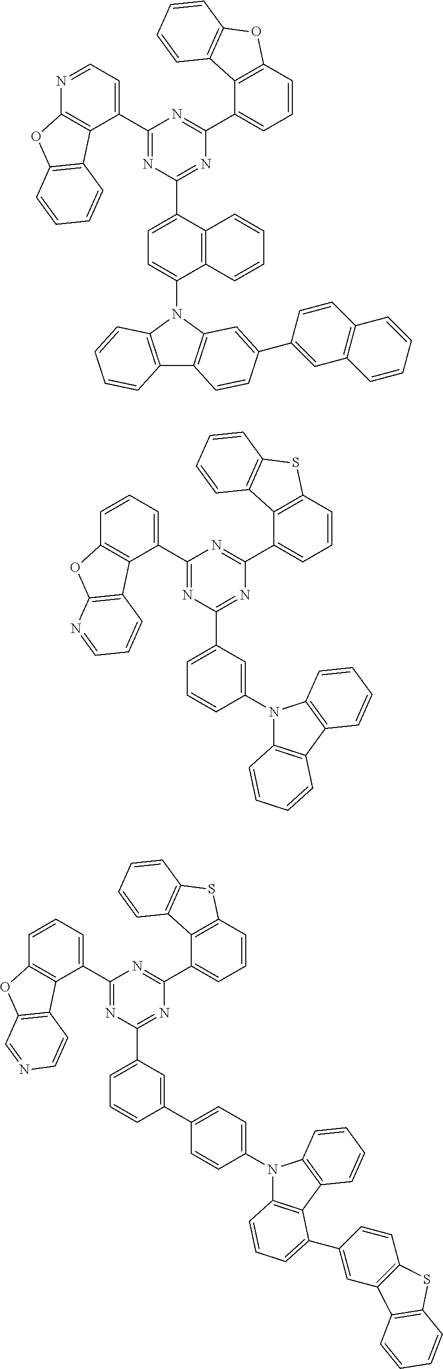

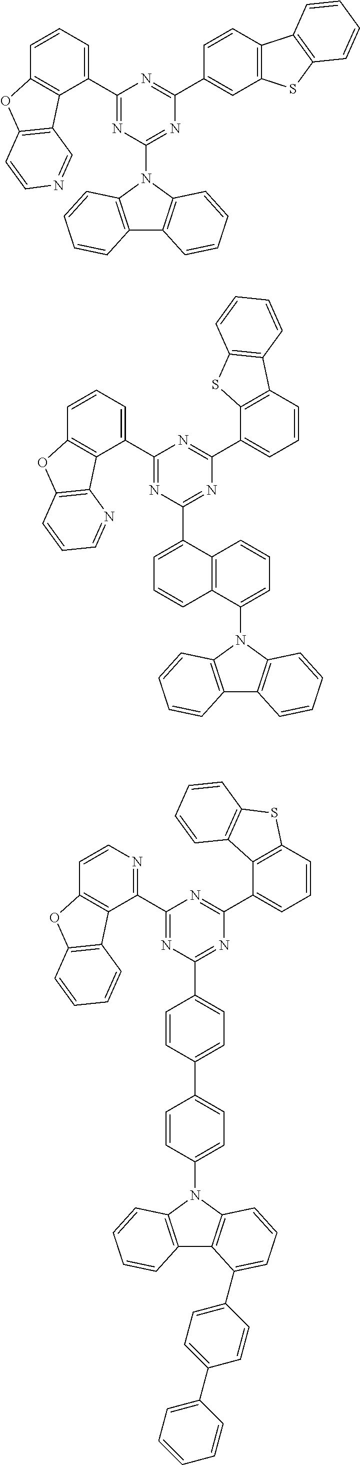 [SMILES: c1cc(-c2nc(-c3ccc4c(c3)sc3ccccc34)nc(-n3c4ccccc4c4ccccc43)n2)c2c(c1)oc1ccncc12 ; c1cnc2c(c1)oc1cccc(-c3nc(-c4cccc5c(-n6c7ccccc7c7ccccc76)cccc45)nc(-c4cccc5c4sc4ccccc45)n3)c12 ; c1ccc(-c2ccc(-c3cccc4c3c3ccccc3n4-c3ccc(-c4ccc(-c5nc(-c6nccc7oc8ccccc8c67)nc(-c6cccc7sc8ccccc8c67)n5)cc4)cc3)cc2)cc1]

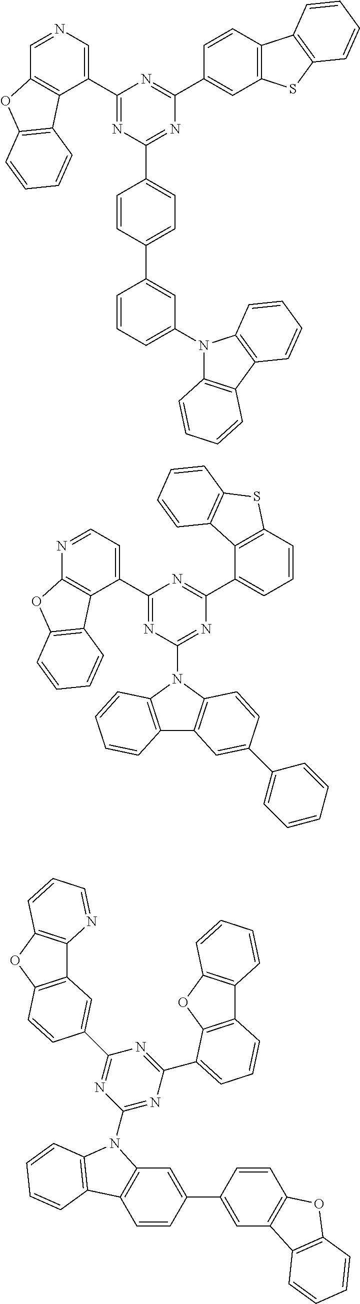

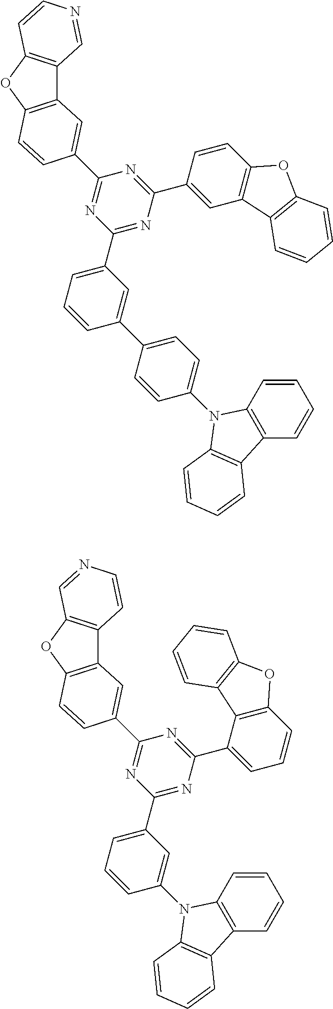 [SMILES: c1cc(-c2ccc(-n3c4ccccc4c4ccccc43)cc2)cc(-c2nc(-c3ccc4oc5ccccc5c4c3)nc(-c3ccc4oc5ccncc5c4c3)n2)c1 ; c1cc(-c2nc(-c3ccc4oc5cnccc5c4c3)nc(-c3cccc4oc5ccccc5c34)n2)cc(-n2c3ccccc3c3ccccc32)c1]

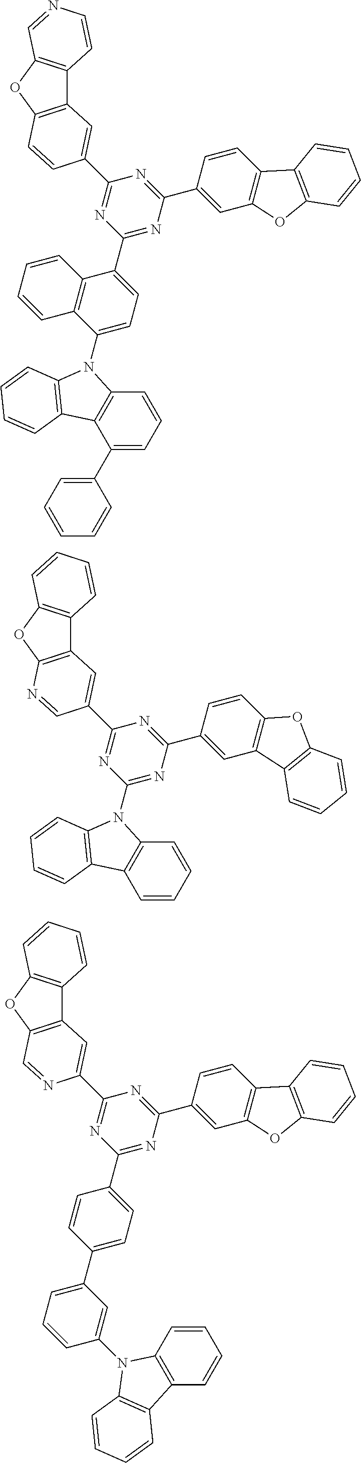

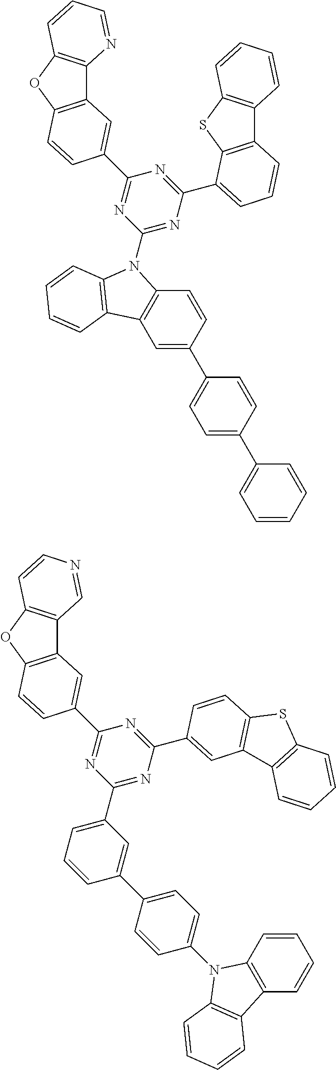 [SMILES: c1ccc(-c2ccc(-c3ccc4c(c3)c3ccccc3n4-c3nc(-c4ccc5oc6cccnc6c5c4)nc(-c4cccc5c4sc4ccccc45)n3)cc2)cc1 ; c1cc(-c2ccc(-n3c4ccccc4c4ccccc43)cc2)cc(-c2nc(-c3ccc4oc5ccncc5c4c3)nc(-c3ccc4sc5ccccc5c4c3)n2)c1]

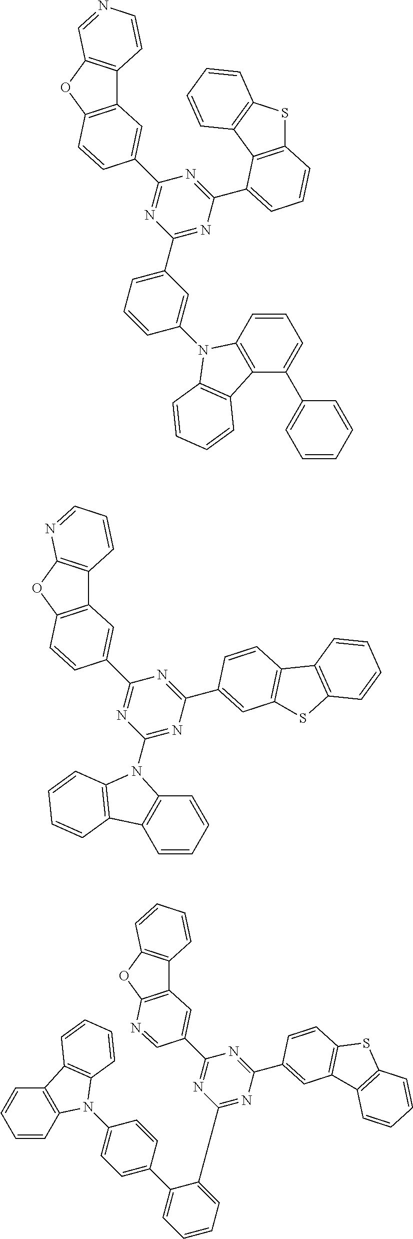 [SMILES: c1ccc(-c2cccc3c2c2ccccc2n3-c2cccc(-c3nc(-c4ccc5oc6cnccc6c5c4)nc(-c4cccc5sc6ccccc6c45)n3)c2)cc1 ; c1ccc2c(c1)sc1cc(-c3nc(-c4ccc5oc6ncccc6c5c4)nc(-n4c5ccccc5c5ccccc54)n3)ccc12 ; c1ccc(-c2nc(-c3cnc4oc5ccccc5c4c3)nc(-c3ccc4sc5ccccc5c4c3)n2)c(-c2ccc(-n3c4ccccc4c4ccccc43)cc2)c1]

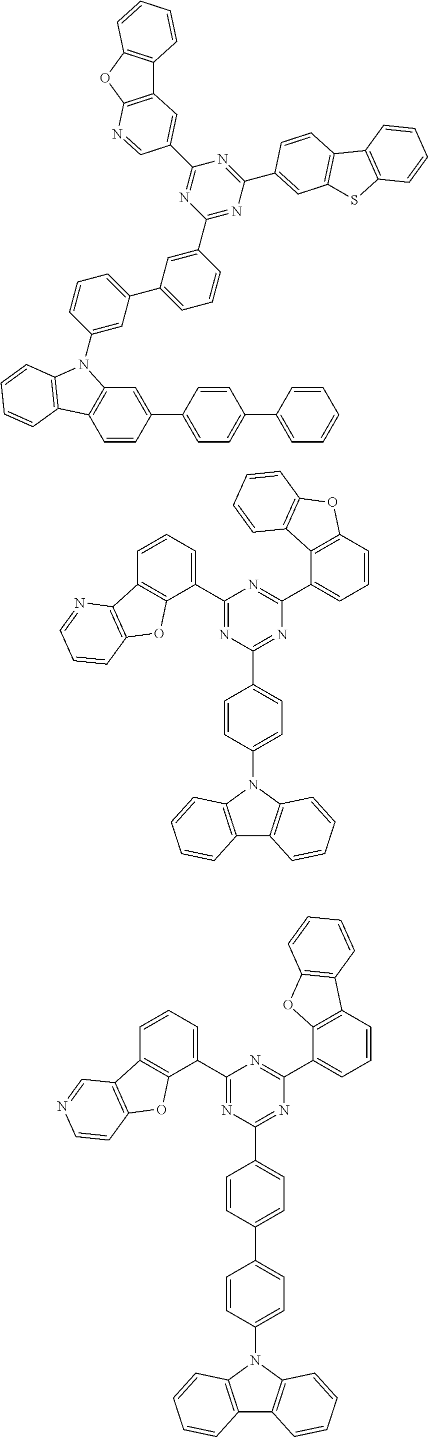

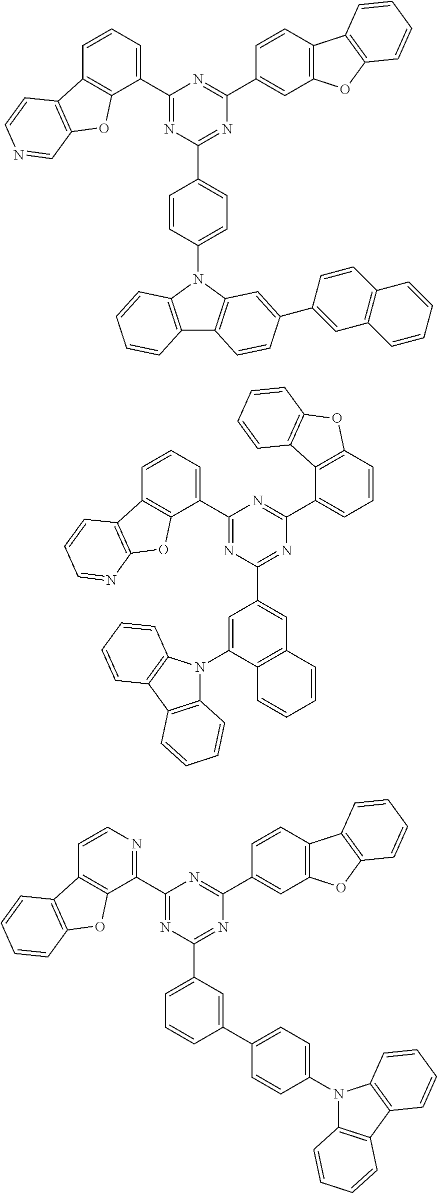

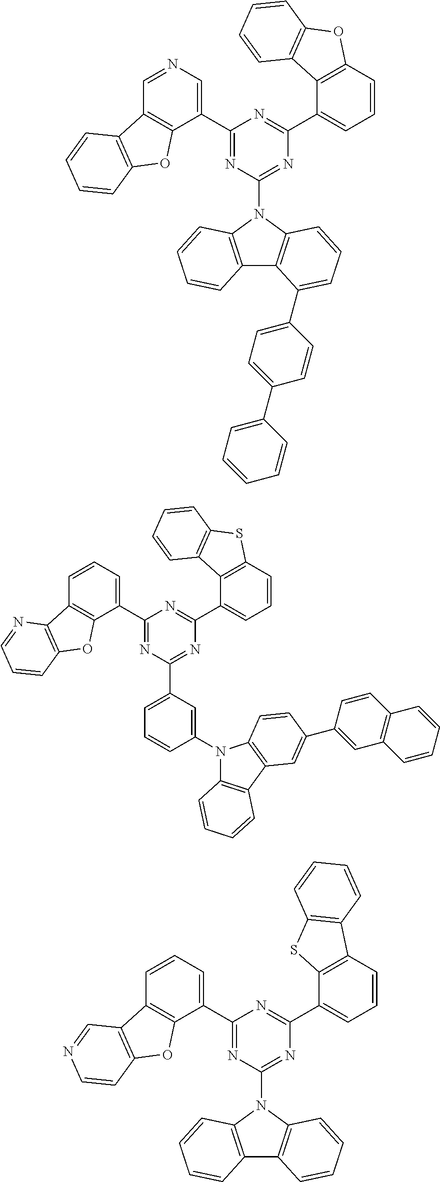 [SMILES: c1ccc(-c2ccc(-c3cccc4c3c3ccccc3n4-c3nc(-c4cncc5c4oc4ccccc45)nc(-c4cccc5oc6ccccc6c45)n3)cc2)cc1 ; c1cc(-c2nc(-c3cccc4c3oc3cccnc34)nc(-c3cccc4sc5ccccc5c34)n2)cc(-n2c3ccccc3c3cc(-c4ccc5ccccc5c4)ccc32)c1 ; c1ccc2c(c1)sc1c(-c3nc(-c4cccc5c4oc4ccncc45)nc(-n4c5ccccc5c5ccccc54)n3)cccc12]

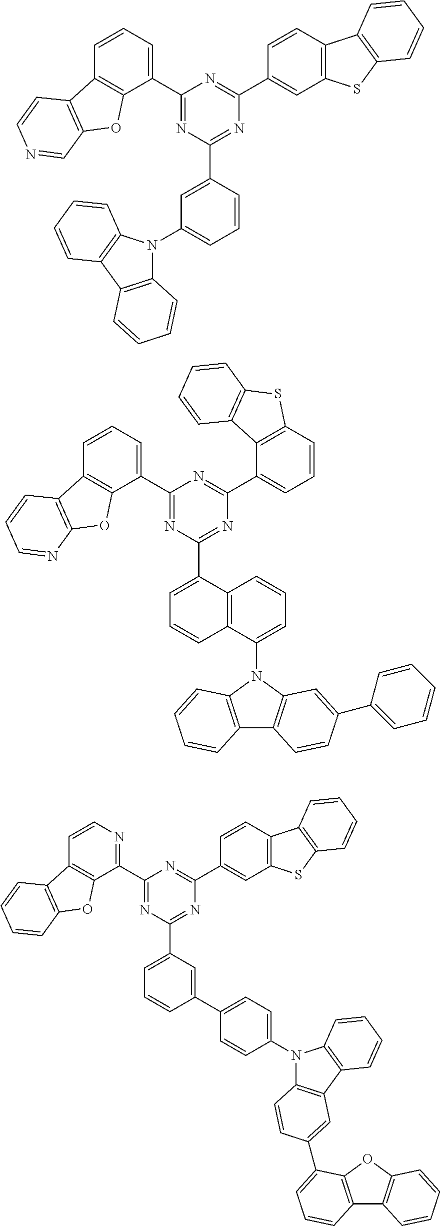

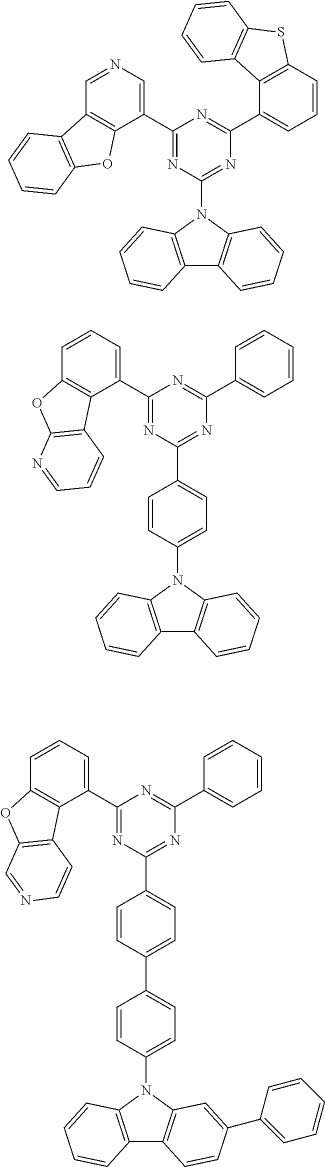

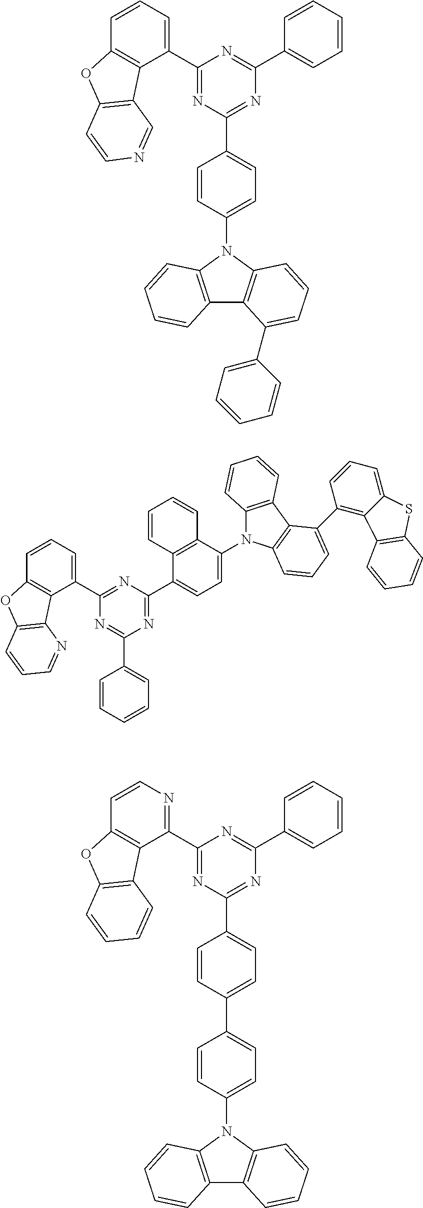

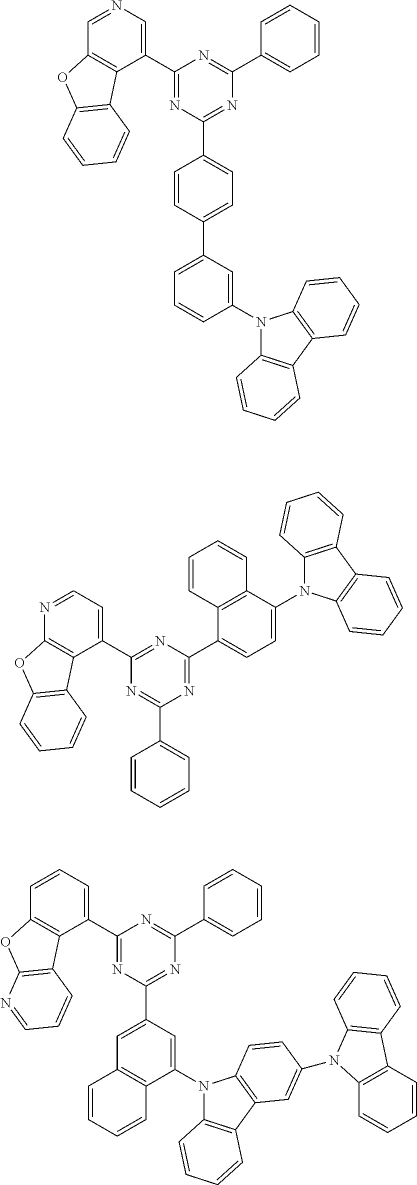 [SMILES: c1ccc(-c2nc(-c3ccc(-c4cccc(-n5c6ccccc6c6ccccc65)c4)cc3)nc(-c3cncc4oc5ccccc5c34)n2)cc1 ; c1ccc(-c2nc(-c3ccc(-n4c5ccccc5c5ccccc54)c4ccccc34)nc(-c3ccnc4oc5ccccc5c34)n2)cc1 ; c1ccc(-c2nc(-c3cc(-n4c5ccccc5c5cc(-n6c7ccccc7c7ccccc76)ccc54)c4ccccc4c3)nc(-c3cccc4oc5ncccc5c34)n2)cc1]

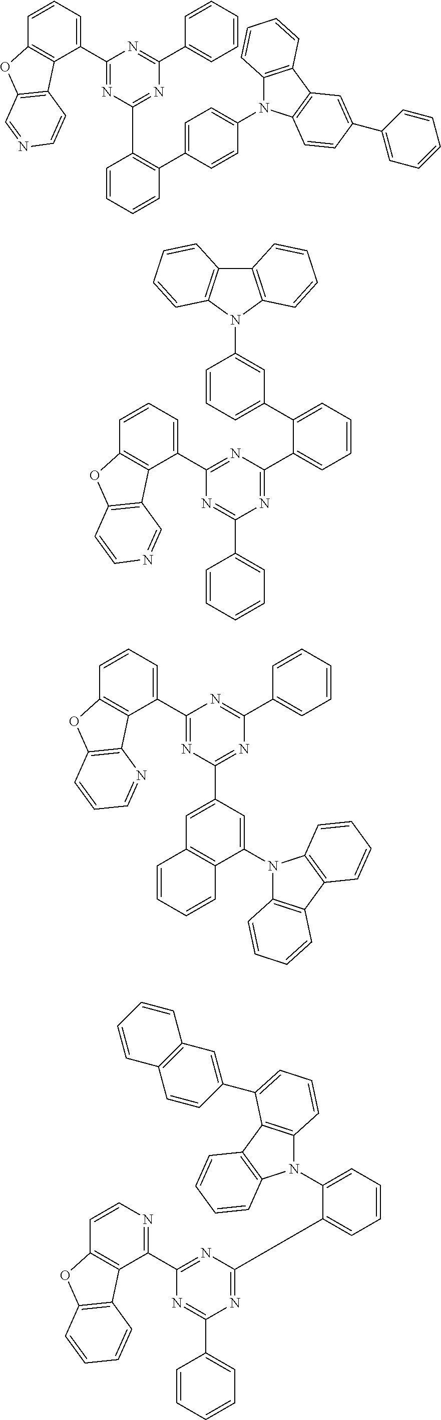 [SMILES: c1ccc(-c2ccc3c(c2)c2ccccc2n3-c2ccc(-c3ccccc3-c3nc(-c4ccccc4)nc(-c4cccc5oc6cnccc6c45)n3)cc2)cc1 ; c1ccc(-c2nc(-c3ccccc3-c3cccc(-n4c5ccccc5c5ccccc54)c3)nc(-c3cccc4oc5ccncc5c34)n2)cc1 ; c1ccc(-c2nc(-c3cc(-n4c5ccccc5c5ccccc54)c4ccccc4c3)nc(-c3cccc4oc5cccnc5c34)n2)cc1 ; c1ccc(-c2nc(-c3ccccc3-n3c4ccccc4c4c(-c5ccc6ccccc6c5)cccc43)nc(-c3nccc4oc5ccccc5c34)n2)cc1]

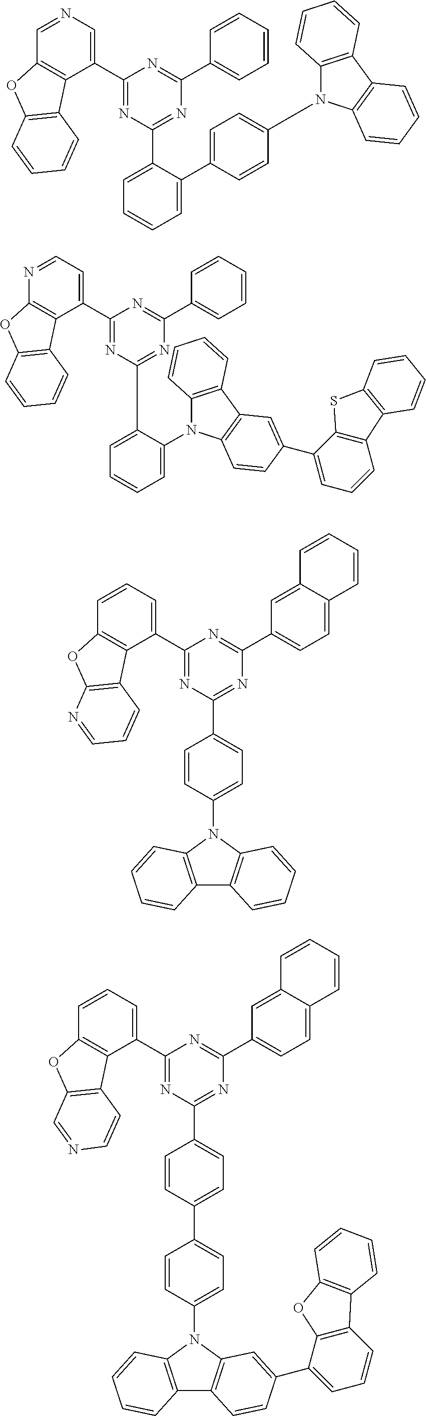

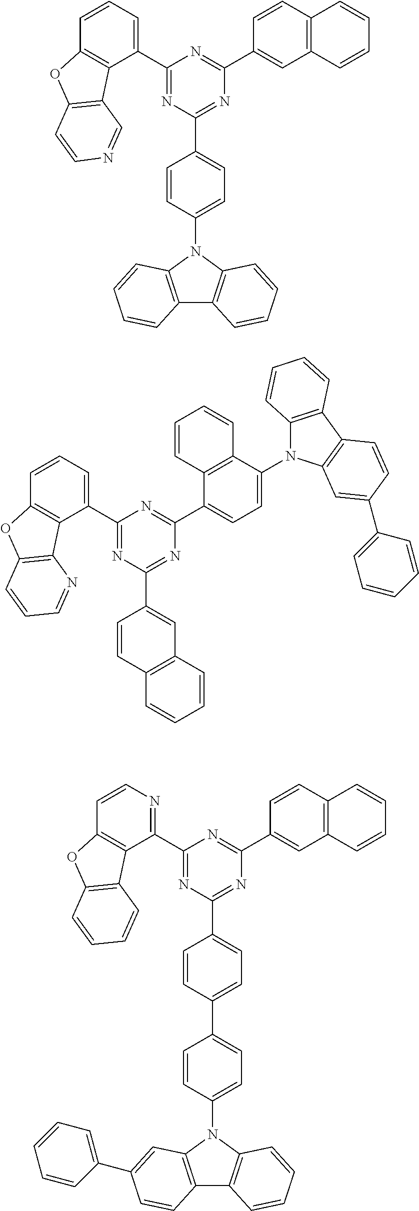 [SMILES: c1ccc2cc(-c3nc(-c4ccc(-n5c6ccccc6c6ccccc65)cc4)nc(-c4cccc5oc6ccncc6c45)n3)ccc2c1 ; c1ccc(-c2ccc3c4ccccc4n(-c4ccc(-c5nc(-c6ccc7ccccc7c6)nc(-c6cccc7oc8cccnc8c67)n5)c5ccccc45)c3c2)cc1 ; c1ccc(-c2ccc3c4ccccc4n(-c4ccc(-c5ccc(-c6nc(-c7ccc8ccccc8c7)nc(-c7nccc8oc9ccccc9c78)n6)cc5)cc4)c3c2)cc1]

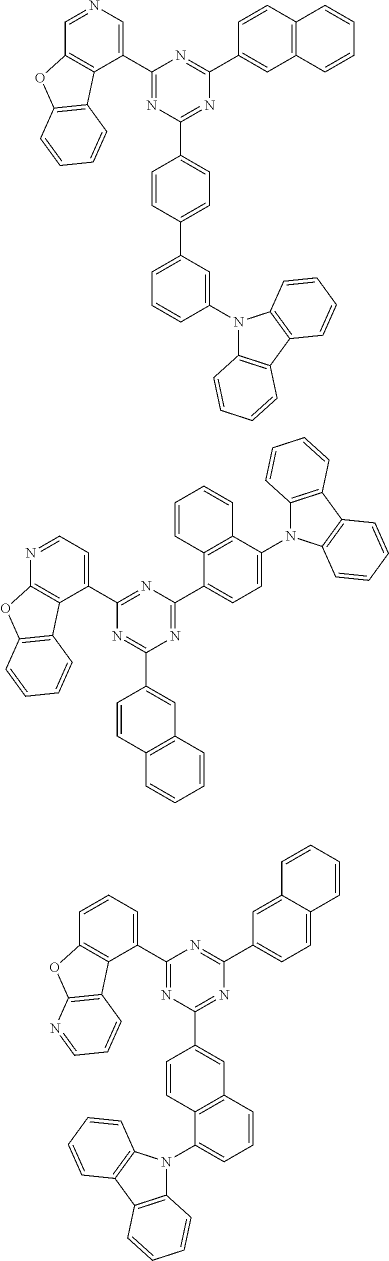

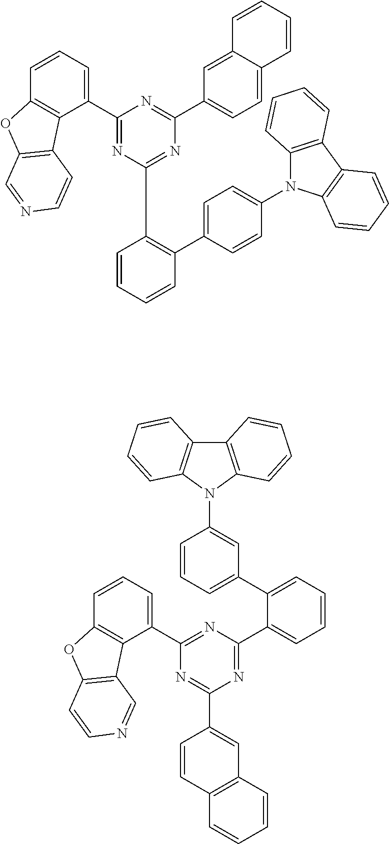 [SMILES: c1ccc(-c2nc(-c3ccc4ccccc4c3)nc(-c3cccc4oc5cnccc5c34)n2)c(-c2ccc(-n3c4ccccc4c4ccccc43)cc2)c1 ; c1cc(-c2ccccc2-c2nc(-c3ccc4ccccc4c3)nc(-c3cccc4oc5ccncc5c34)n2)cc(-n2c3ccccc3c3ccccc32)c1]

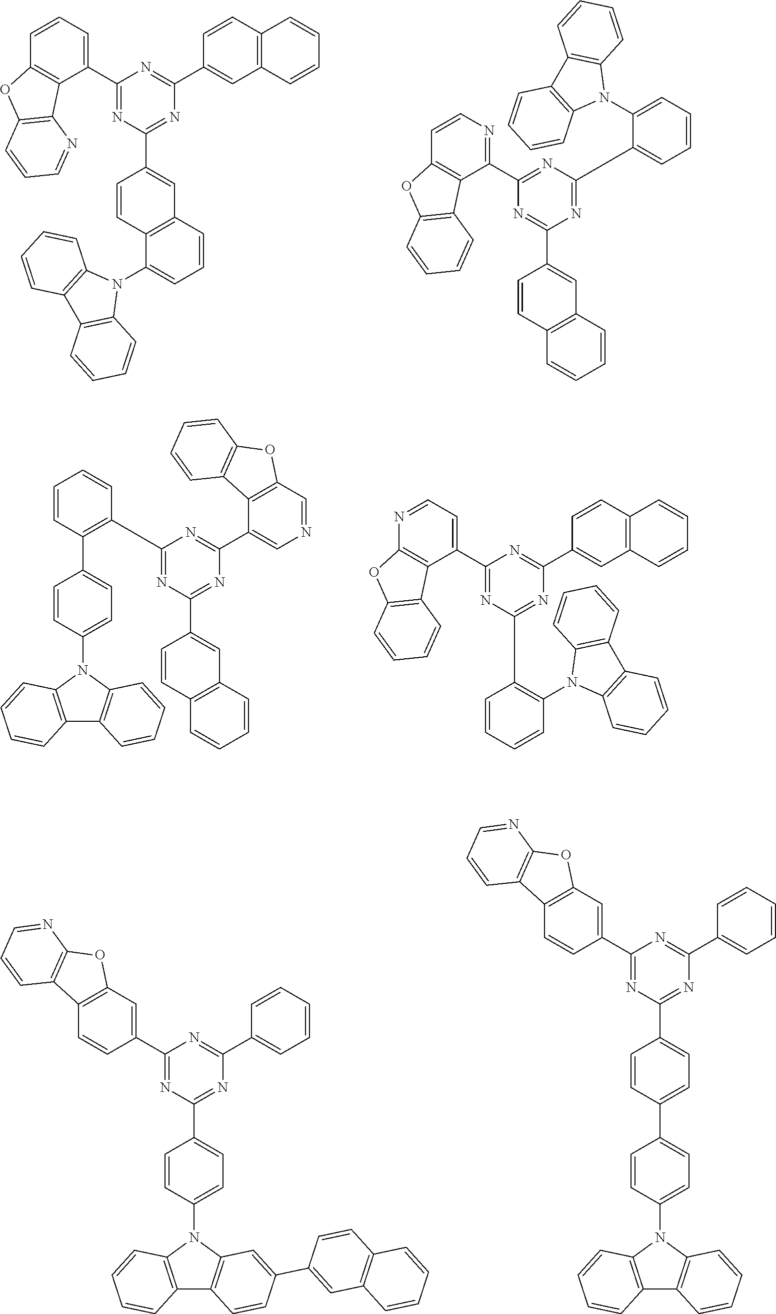

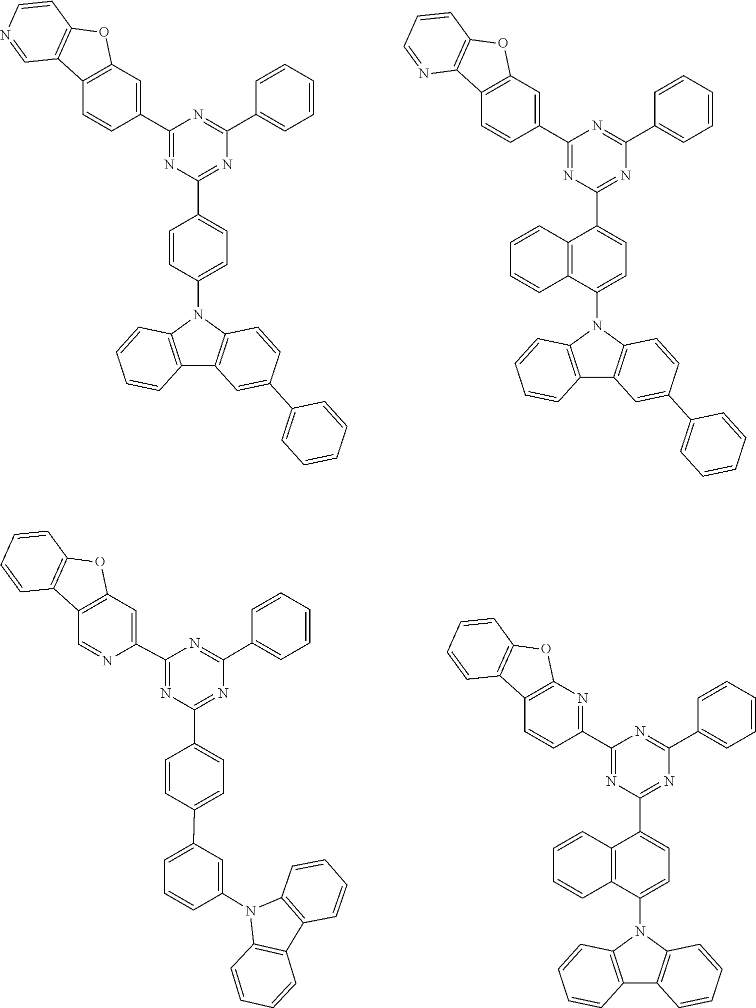 [SMILES: c1ccc(-c2ccc3c(c2)c2ccccc2n3-c2ccc(-c3nc(-c4ccccc4)nc(-c4ccc5c(c4)oc4ccncc45)n3)cc2)cc1 ; c1ccc(-c2ccc3c(c2)c2ccccc2n3-c2ccc(-c3nc(-c4ccccc4)nc(-c4ccc5c(c4)oc4cccnc45)n3)c3ccccc23)cc1 ; c1ccc(-c2nc(-c3ccc(-c4cccc(-n5c6ccccc6c6ccccc65)c4)cc3)nc(-c3cc4oc5ccccc5c4cn3)n2)cc1 ; c1ccc(-c2nc(-c3ccc4c(n3)oc3ccccc34)nc(-c3ccc(-n4c5ccccc5c5ccccc54)c4ccccc34)n2)cc1]

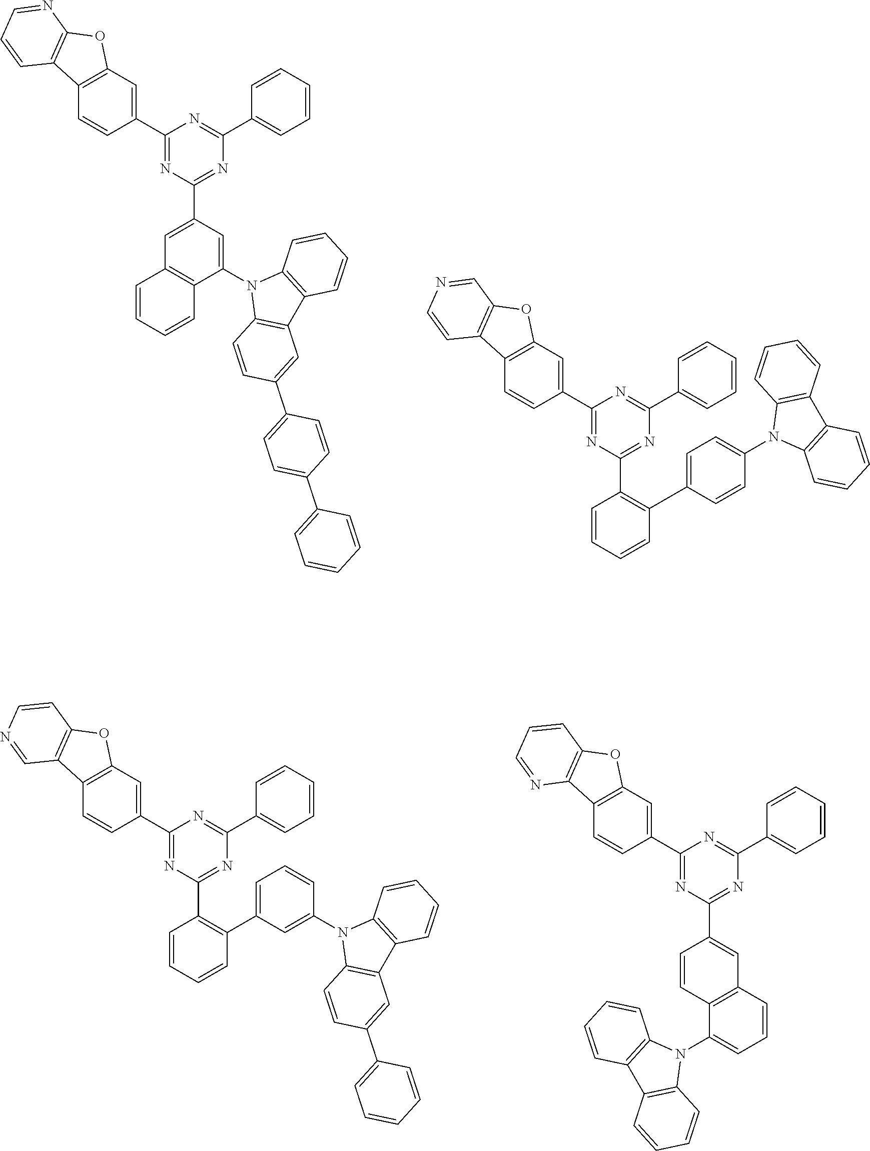 [SMILES: c1ccc(-c2ccc(-c3ccc4c(c3)c3ccccc3n4-c3cc(-c4nc(-c5ccccc5)nc(-c5ccc6c(c5)oc5ncccc56)n4)cc4ccccc34)cc2)cc1 ; c1ccc(-c2nc(-c3ccc4c(c3)oc3cnccc34)nc(-c3ccccc3-c3ccc(-n4c5ccccc5c5ccccc54)cc3)n2)cc1 ; c1ccc(-c2ccc3c(c2)c2ccccc2n3-c2cccc(-c3ccccc3-c3nc(-c4ccccc4)nc(-c4ccc5c(c4)oc4ccncc45)n3)c2)cc1 ; c1ccc(-c2nc(-c3ccc4c(-n5c6ccccc6c6ccccc65)cccc4c3)nc(-c3ccc4c(c3)oc3cccnc34)n2)cc1]

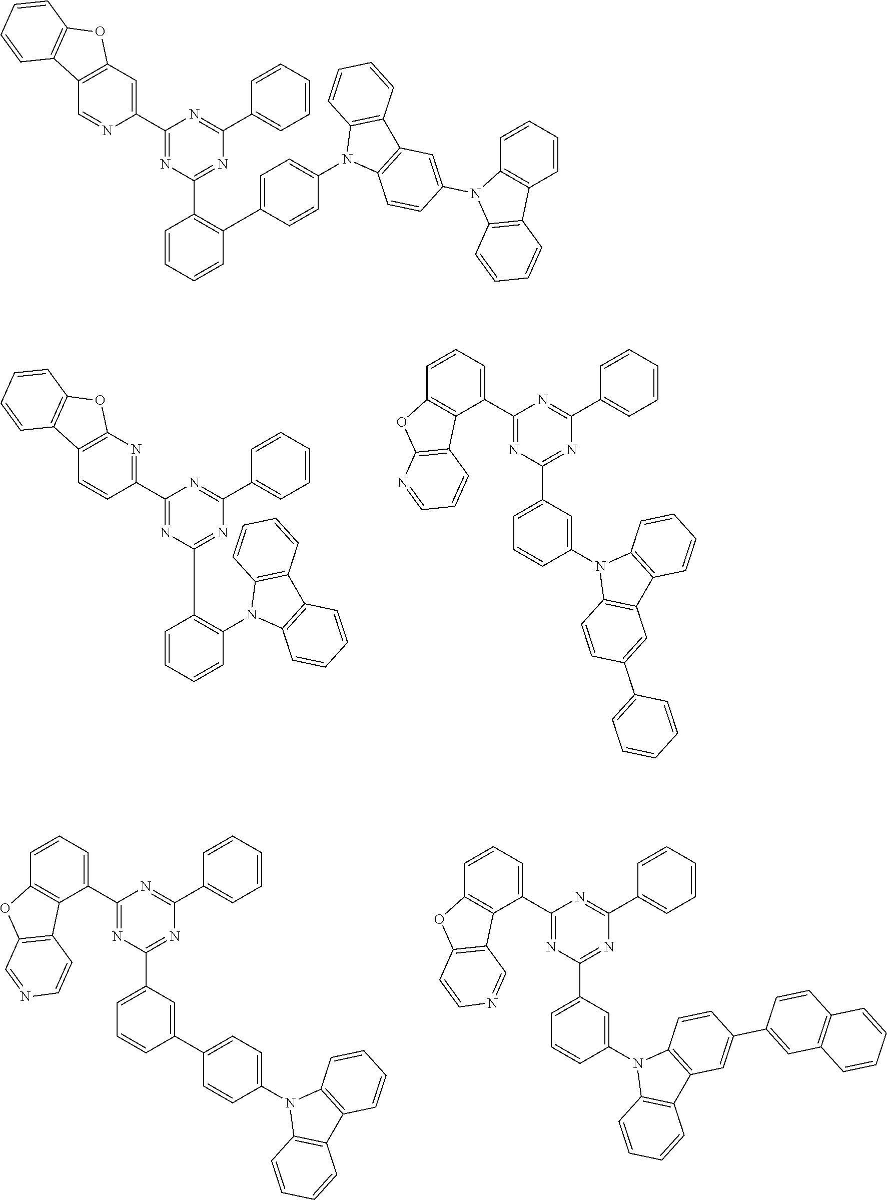 [SMILES: c1ccc(-c2nc(-c3cc4oc5ccccc5c4cn3)nc(-c3ccccc3-c3ccc(-n4c5ccccc5c5cc(-n6c7ccccc7c7ccccc76)ccc54)cc3)n2)cc1 ; c1ccc(-c2nc(-c3ccc4c(n3)oc3ccccc34)nc(-c3ccccc3-n3c4ccccc4c4ccccc43)n2)cc1 ; c1ccc(-c2ccc3c(c2)c2ccccc2n3-c2cccc(-c3nc(-c4ccccc4)nc(-c4cccc5oc6ncccc6c45)n3)c2)cc1 ; c1ccc(-c2nc(-c3cccc(-c4ccc(-n5c6ccccc6c6ccccc65)cc4)c3)nc(-c3cccc4oc5cnccc5c34)n2)cc1 ; c1ccc(-c2nc(-c3cccc(-n4c5ccccc5c5cc(-c6ccc7ccccc7c6)ccc54)c3)nc(-c3cccc4oc5ccncc5c34)n2)cc1]

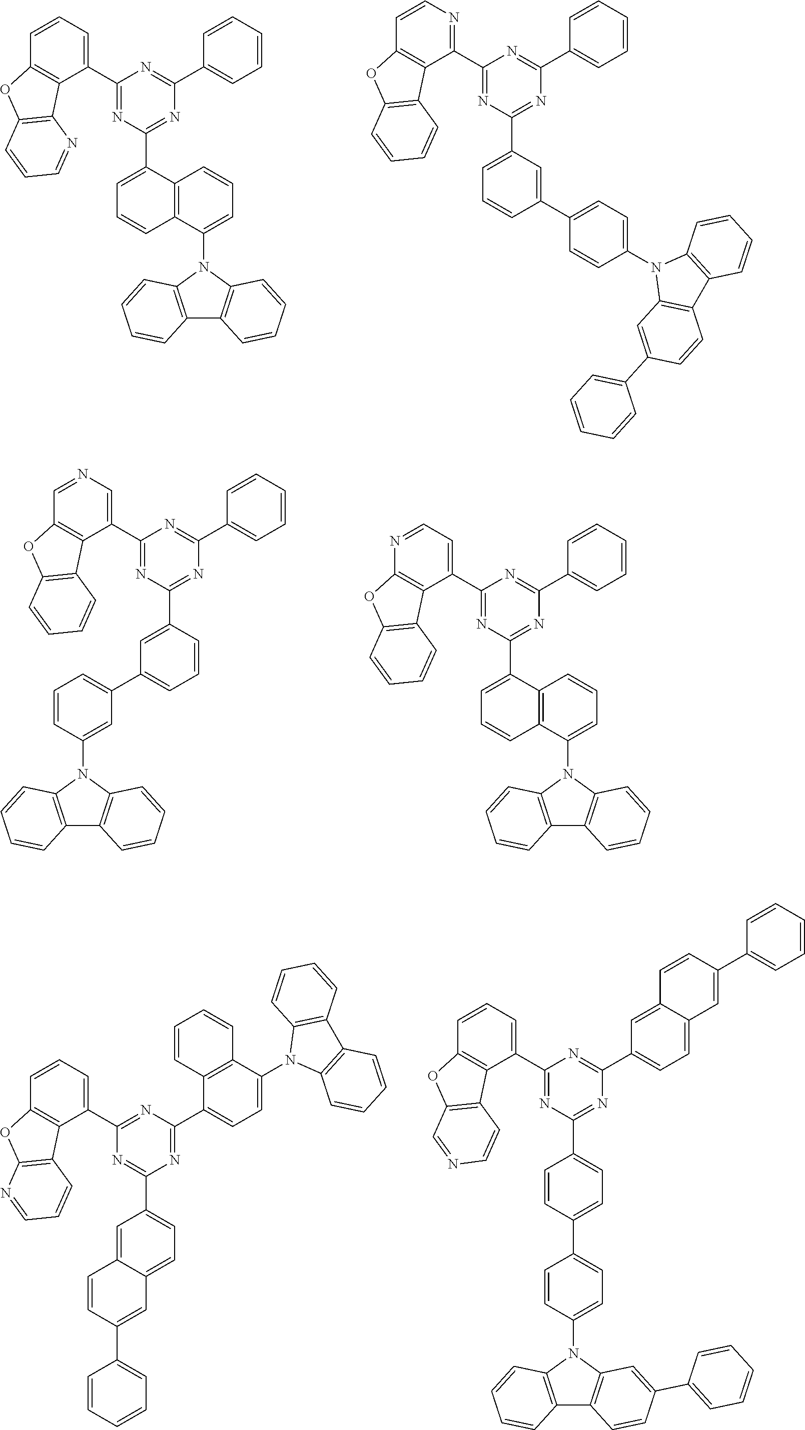

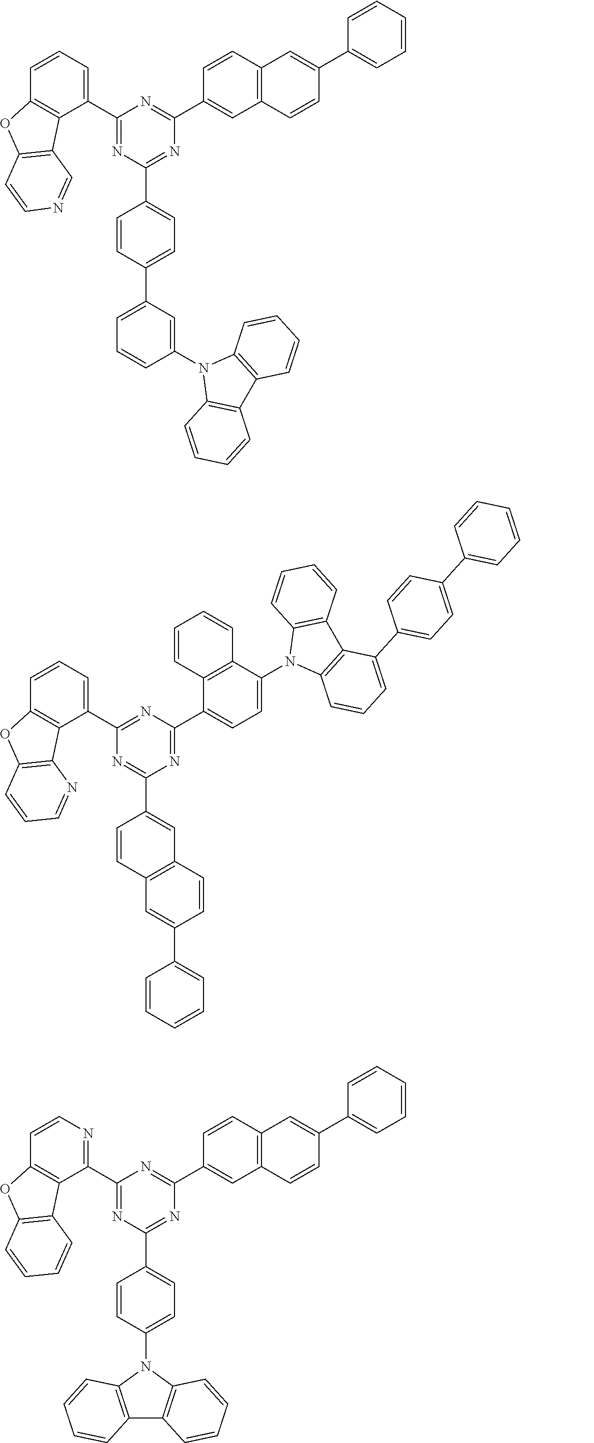 [SMILES: c1ccc(-c2ccc3cc(-c4nc(-c5ccc(-c6cccc(-n7c8ccccc8c8ccccc87)c6)cc5)nc(-c5cccc6oc7ccncc7c56)n4)ccc3c2)cc1 ; c1ccc(-c2ccc(-c3cccc4c3c3ccccc3n4-c3ccc(-c4nc(-c5ccc6cc(-c7ccccc7)ccc6c5)nc(-c5cccc6oc7cccnc7c56)n4)c4ccccc34)cc2)cc1 ; c1ccc(-c2ccc3cc(-c4nc(-c5ccc(-n6c7ccccc7c7ccccc76)cc5)nc(-c5nccc6oc7ccccc7c56)n4)ccc3c2)cc1]

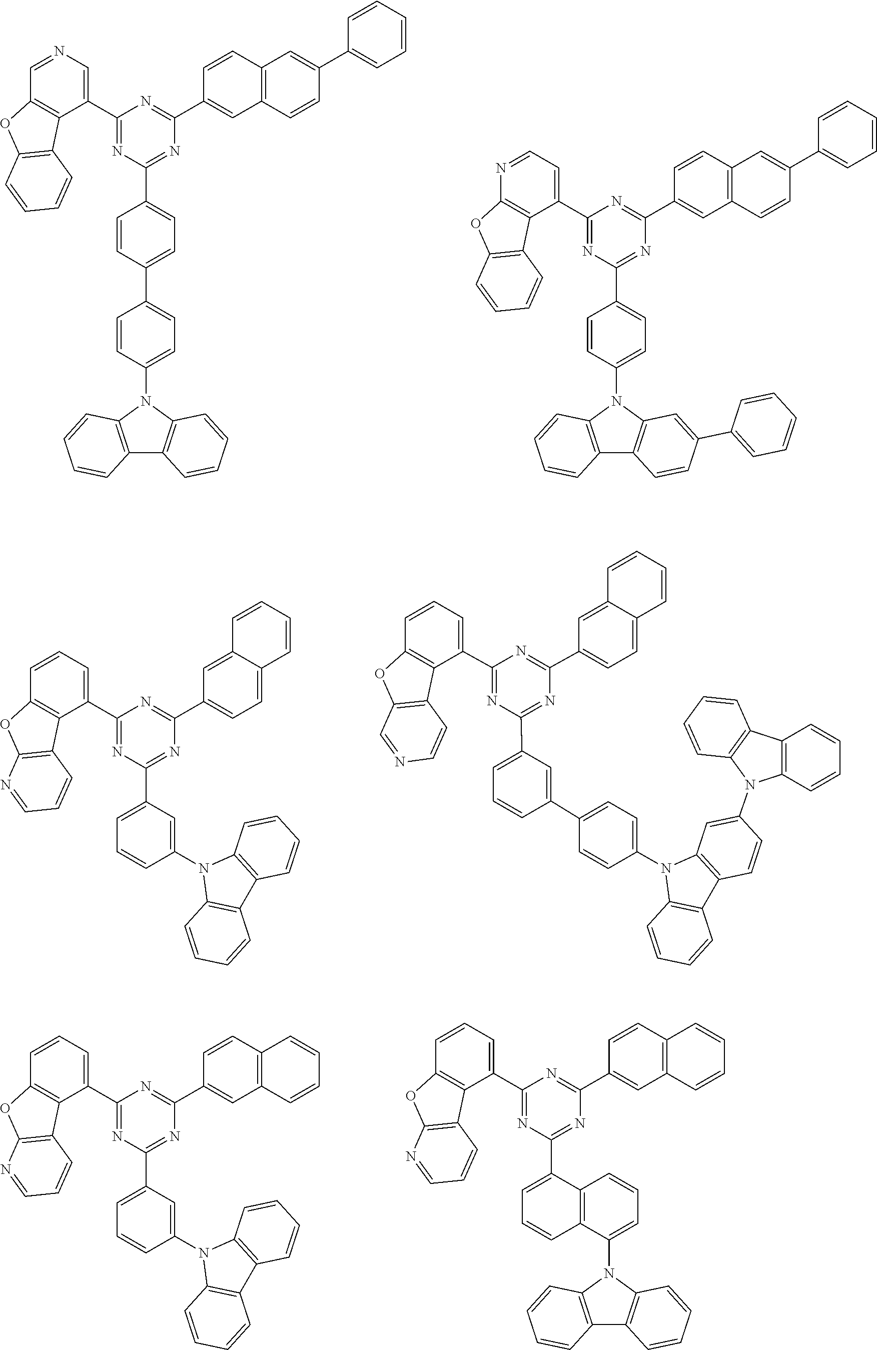

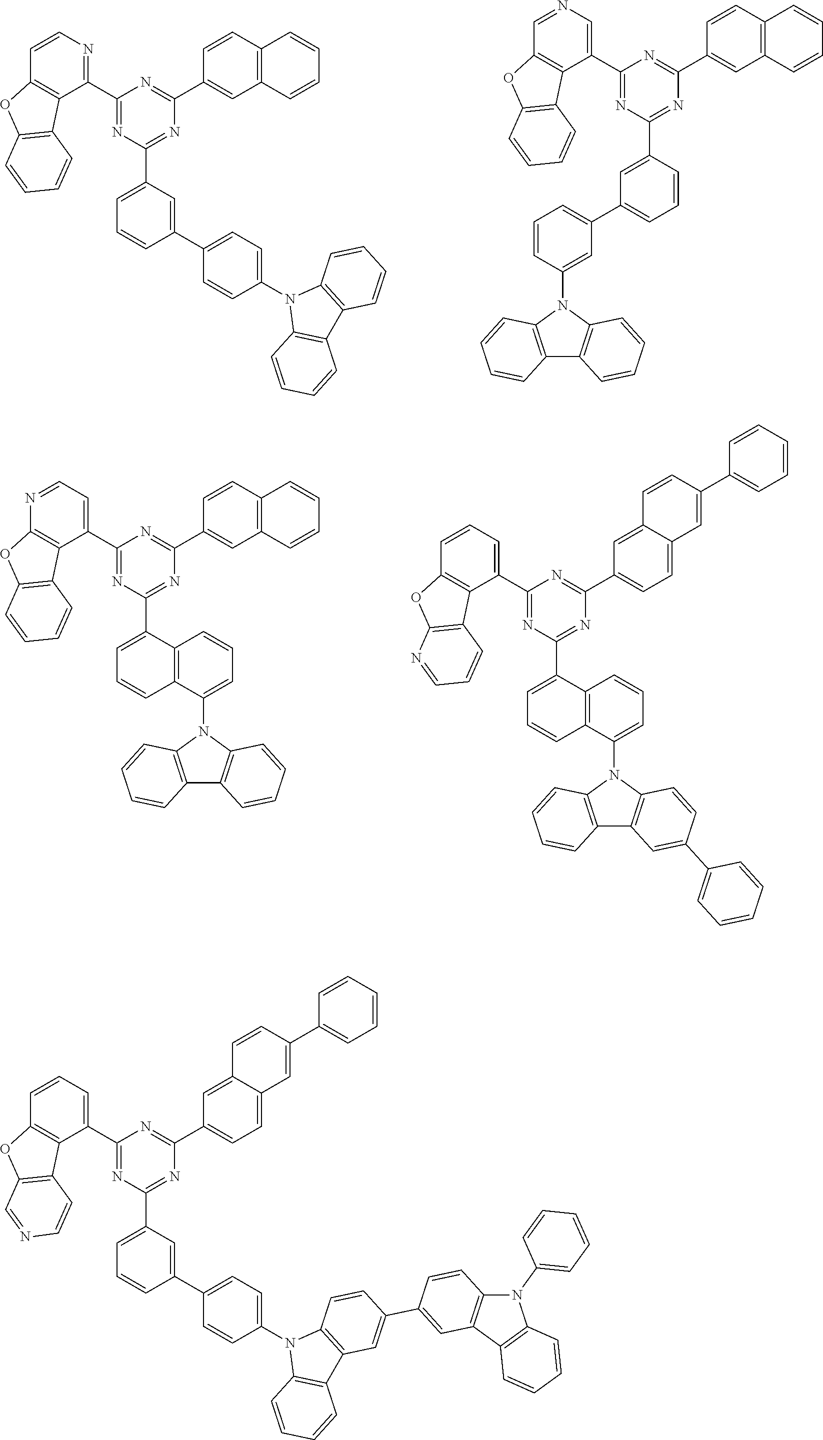

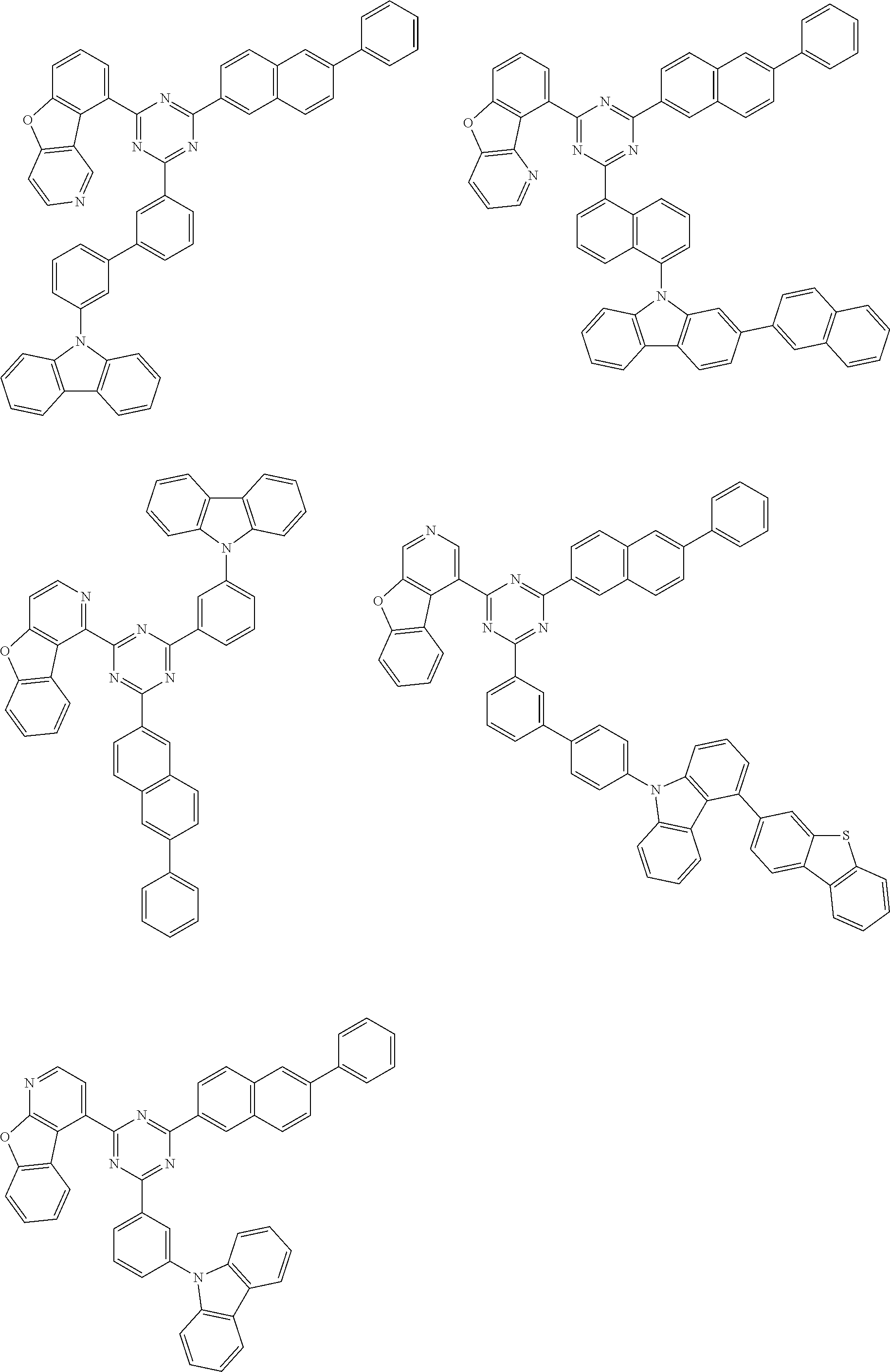

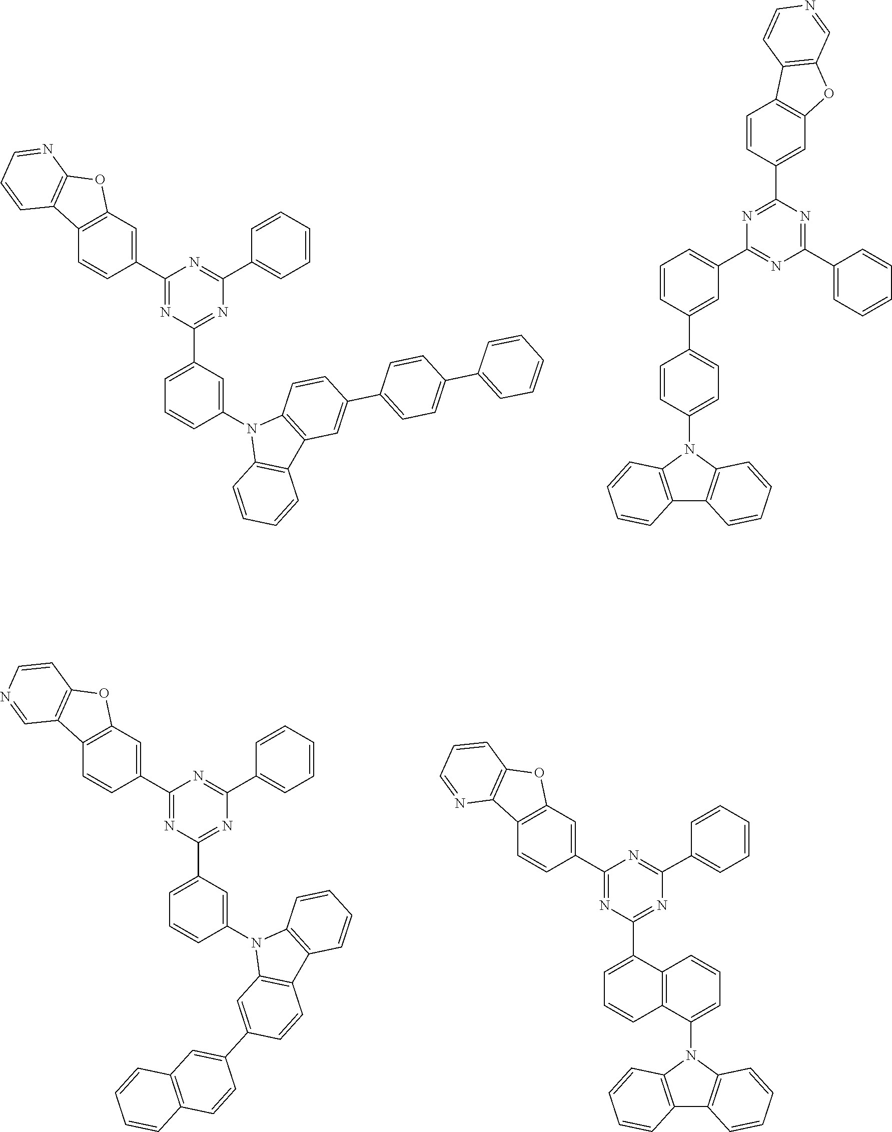 [SMILES: c1ccc(-c2ccc(-c3ccc4c(c3)c3ccccc3n4-c3cccc(-c4nc(-c5ccccc5)nc(-c5ccc6c(c5)oc5ncccc56)n4)c3)cc2)cc1 ; c1ccc(-c2nc(-c3cccc(-c4ccc(-n5c6ccccc6c6ccccc65)cc4)c3)nc(-c3ccc4c(c3)oc3cnccc34)n2)cc1 ; c1ccc(-c2nc(-c3cccc(-n4c5ccccc5c5ccc(-c6ccc7ccccc7c6)cc54)c3)nc(-c3ccc4c(c3)oc3ccncc34)n2)cc1 ; c1ccc(-c2nc(-c3ccc4c(c3)oc3cccnc34)nc(-c3cccc4c(-n5c6ccccc6c6ccccc65)cccc34)n2)cc1]

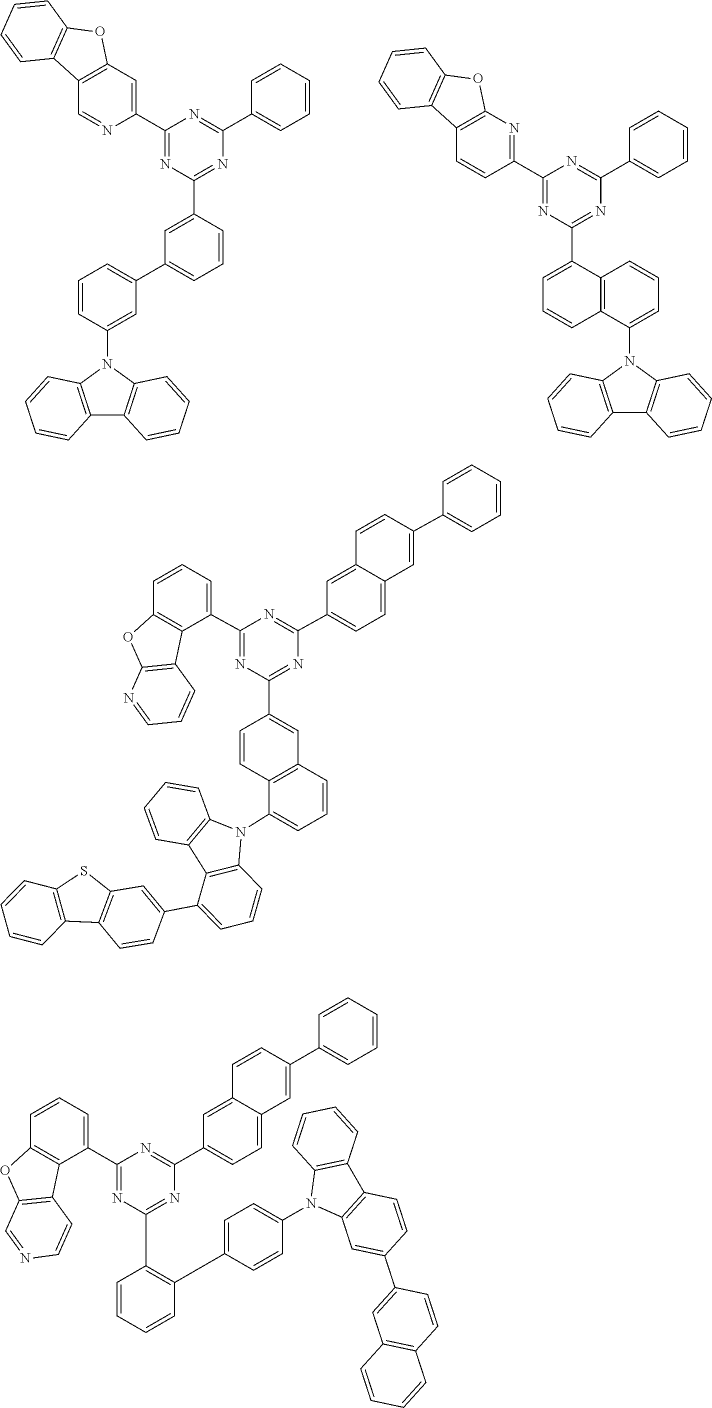 [SMILES: c1ccc(-c2nc(-c3cccc(-c4cccc(-n5c6ccccc6c6ccccc65)c4)c3)nc(-c3cc4oc5ccccc5c4cn3)n2)cc1 ; c1ccc(-c2nc(-c3ccc4c(n3)oc3ccccc34)nc(-c3cccc4c(-n5c6ccccc6c6ccccc65)cccc34)n2)cc1 ; c1ccc(-c2ccc3cc(-c4nc(-c5ccc6c(-n7c8ccccc8c8c(-c9ccc%10c(c9)sc9ccccc9%10)cccc87)cccc6c5)nc(-c5cccc6oc7ncccc7c56)n4)ccc3c2)cc1 ; c1ccc(-c2ccc3cc(-c4nc(-c5ccccc5-c5ccc(-n6c7ccccc7c7ccc(-c8ccc9ccccc9c8)cc76)cc5)nc(-c5cccc6oc7cnccc7c56)n4)ccc3c2)cc1]

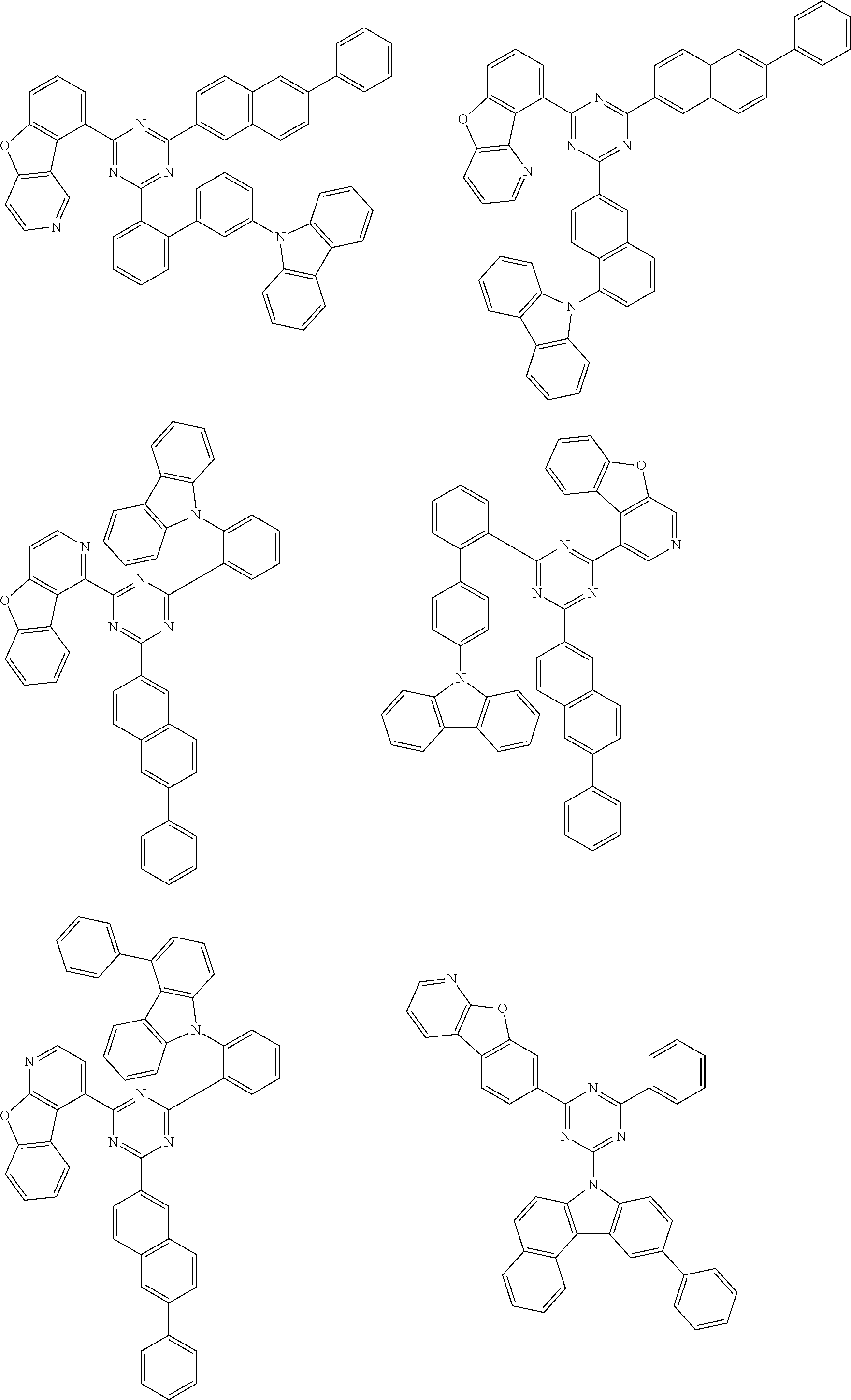

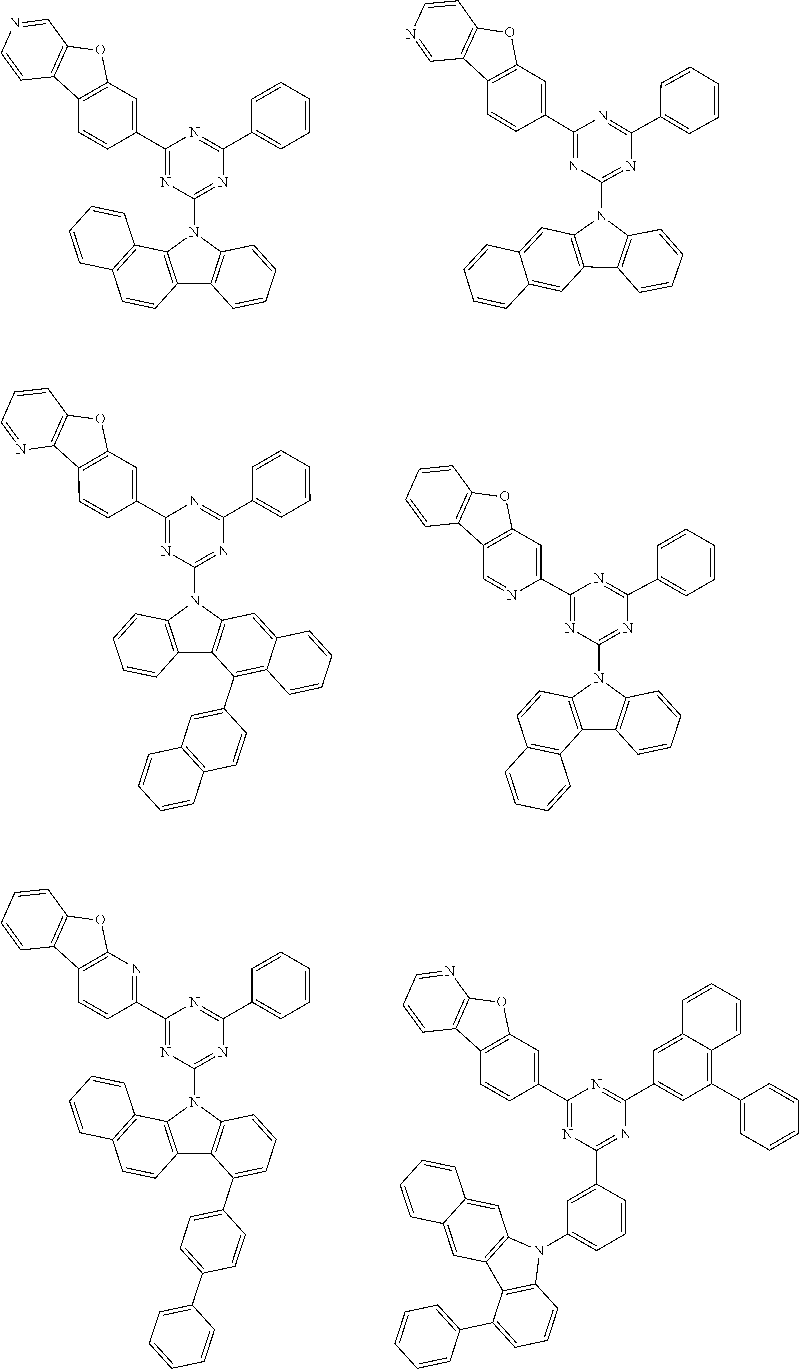

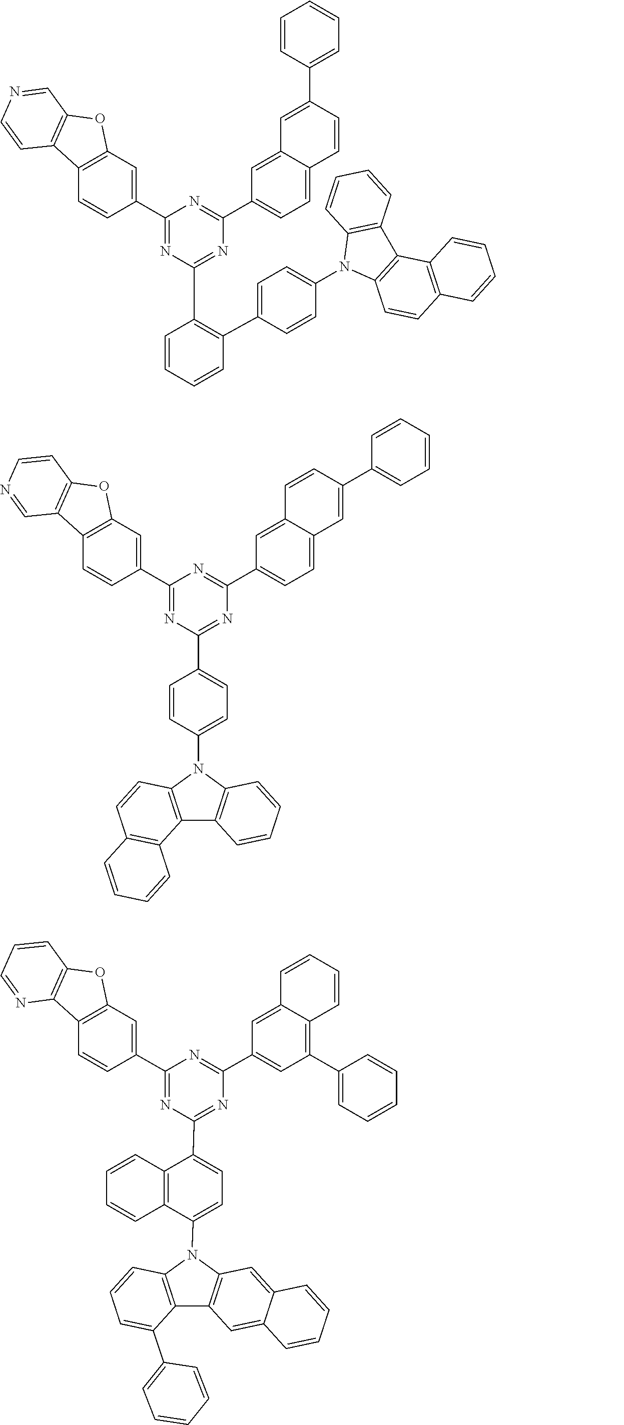 [SMILES: c1ccc(-c2ccc3ccc(-c4nc(-c5ccc6c(c5)oc5cnccc56)nc(-c5ccccc5-c5ccc(-n6c7ccccc7c7c8ccccc8ccc76)cc5)n4)cc3c2)cc1 ; c1ccc(-c2ccc3cc(-c4nc(-c5ccc(-n6c7ccccc7c7c8ccccc8ccc76)cc5)nc(-c5ccc6c(c5)oc5ccncc56)n4)ccc3c2)cc1 ; c1ccc(-c2cc(-c3nc(-c4ccc5c(c4)oc4cccnc45)nc(-c4ccc(-n5c6cc7ccccc7cc6c6c(-c7ccccc7)cccc65)c5ccccc45)n3)cc3ccccc23)cc1]

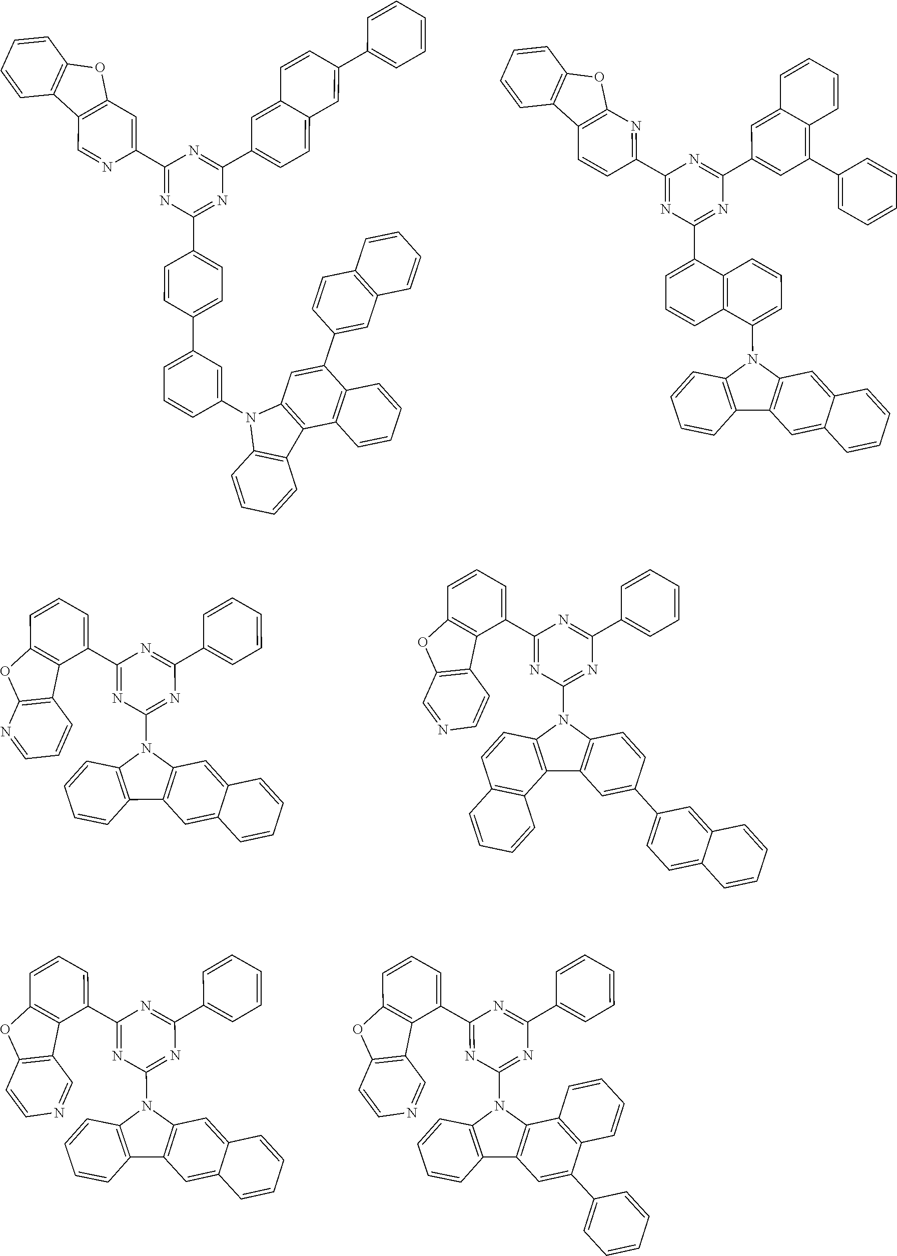

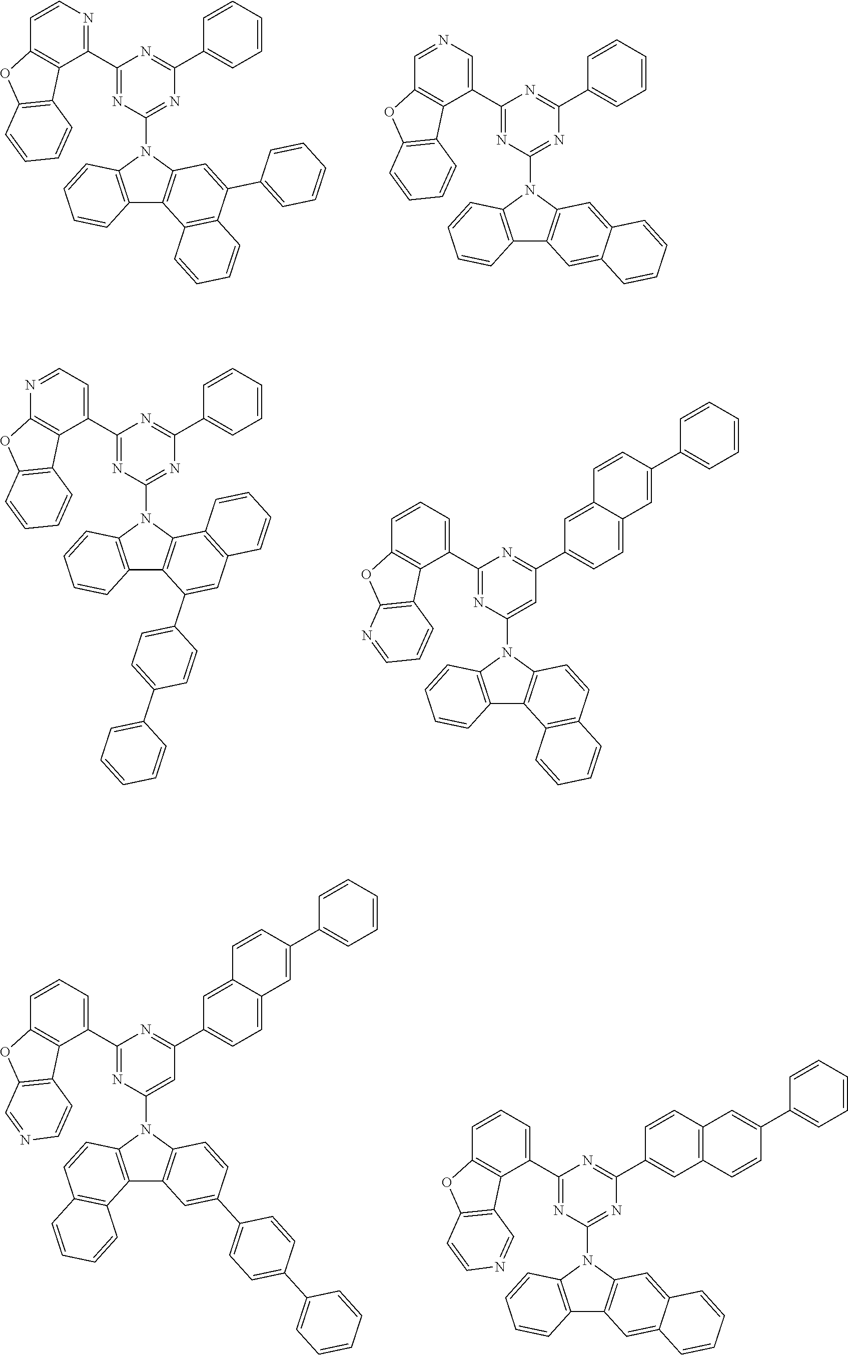

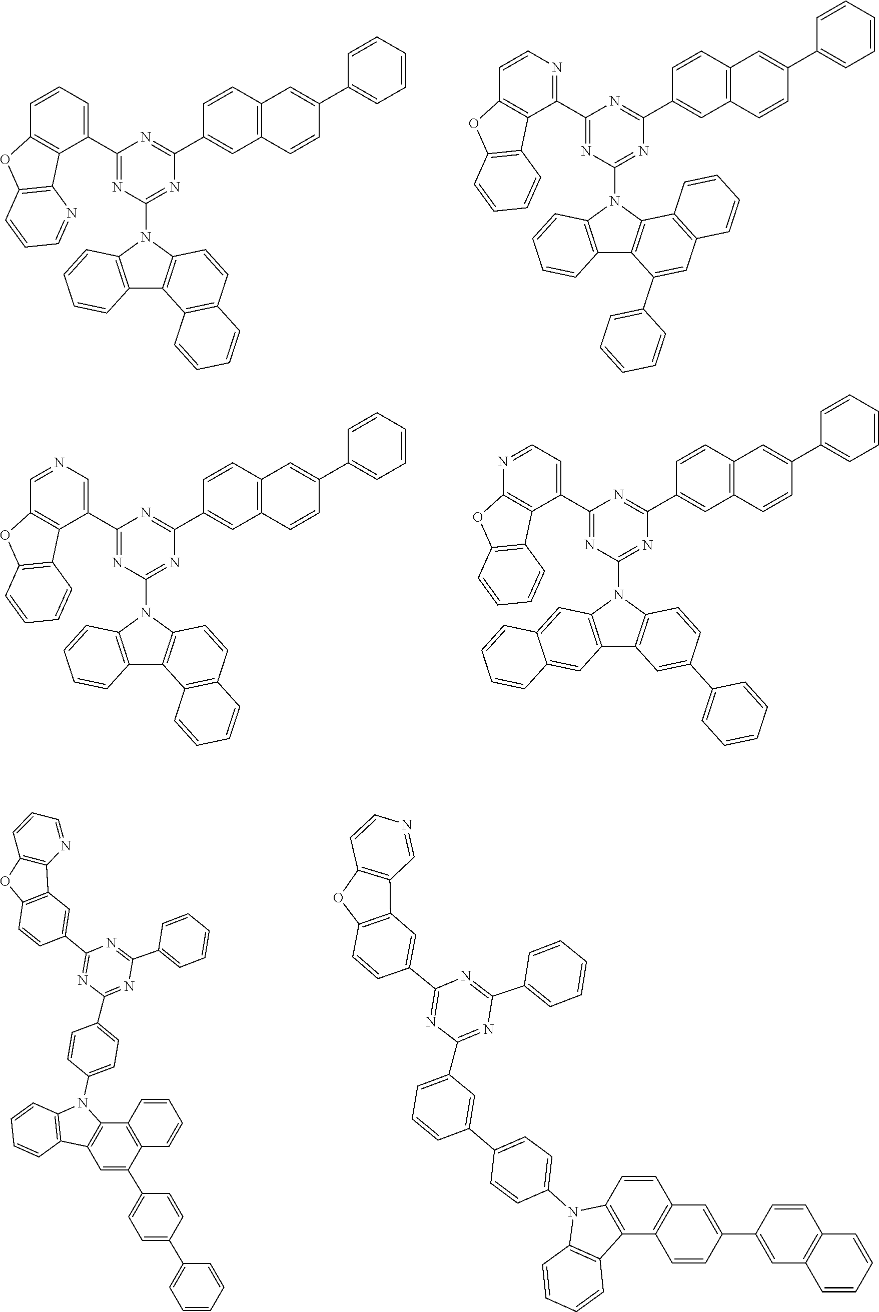

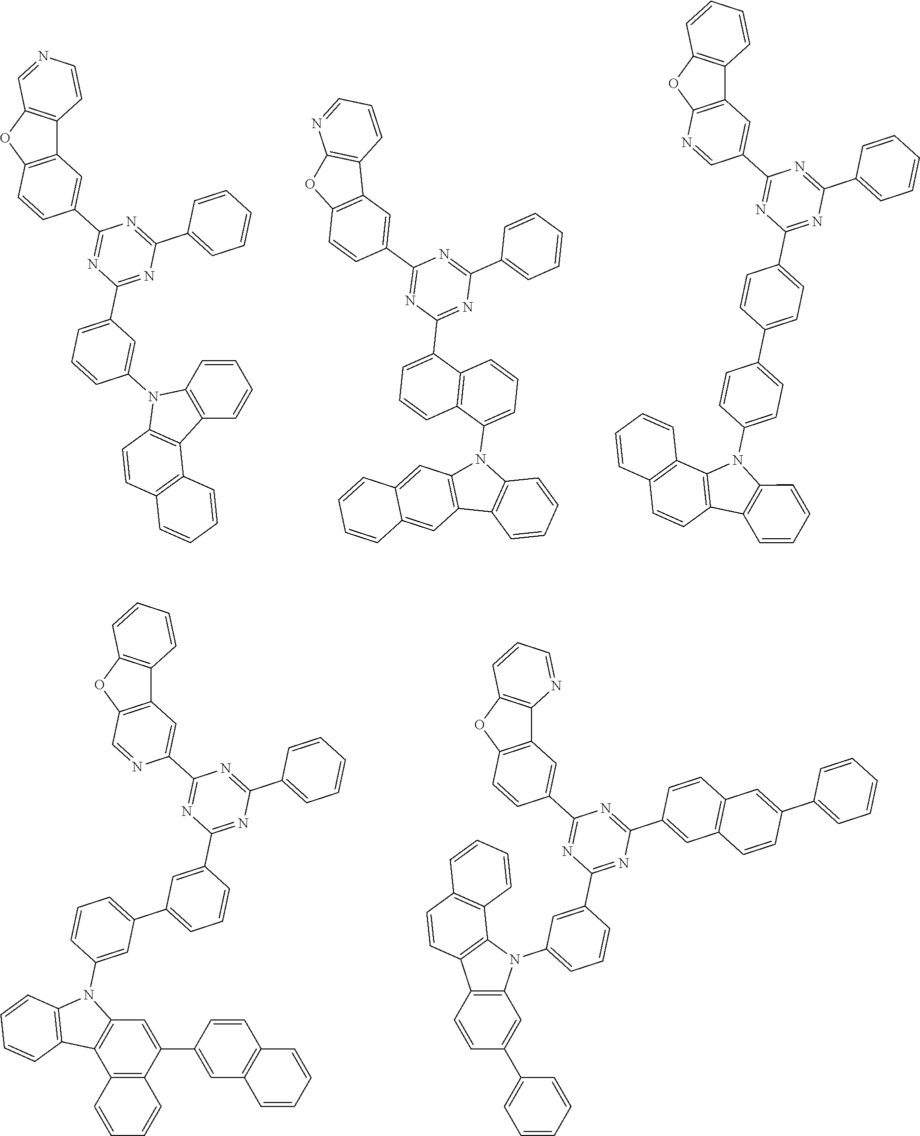 [SMILES: c1ccc(-c2nc(-c3cccc(-n4c5ccccc5c5c6ccccc6ccc54)c3)nc(-c3ccc4oc5cnccc5c4c3)n2)cc1 ; c1ccc(-c2nc(-c3ccc4oc5ncccc5c4c3)nc(-c3cccc4c(-n5c6ccccc6c6cc7ccccc7cc65)cccc34)n2)cc1 ; c1ccc(-c2nc(-c3ccc(-c4ccc(-n5c6ccccc6c6ccc7ccccc7c65)cc4)cc3)nc(-c3cnc4oc5ccccc5c4c3)n2)cc1 ; c1ccc(-c2nc(-c3cccc(-c4cccc(-n5c6ccccc6c6c7ccccc7c(-c7ccc8ccccc8c7)cc65)c4)c3)nc(-c3cc4c(cn3)oc3ccccc34)n2)cc1 ; c1ccc(-c2ccc3cc(-c4nc(-c5cccc(-n6c7cc(-c8ccccc8)ccc7c7ccc8ccccc8c76)c5)nc(-c5ccc6oc7cccnc7c6c5)n4)ccc3c2)cc1]

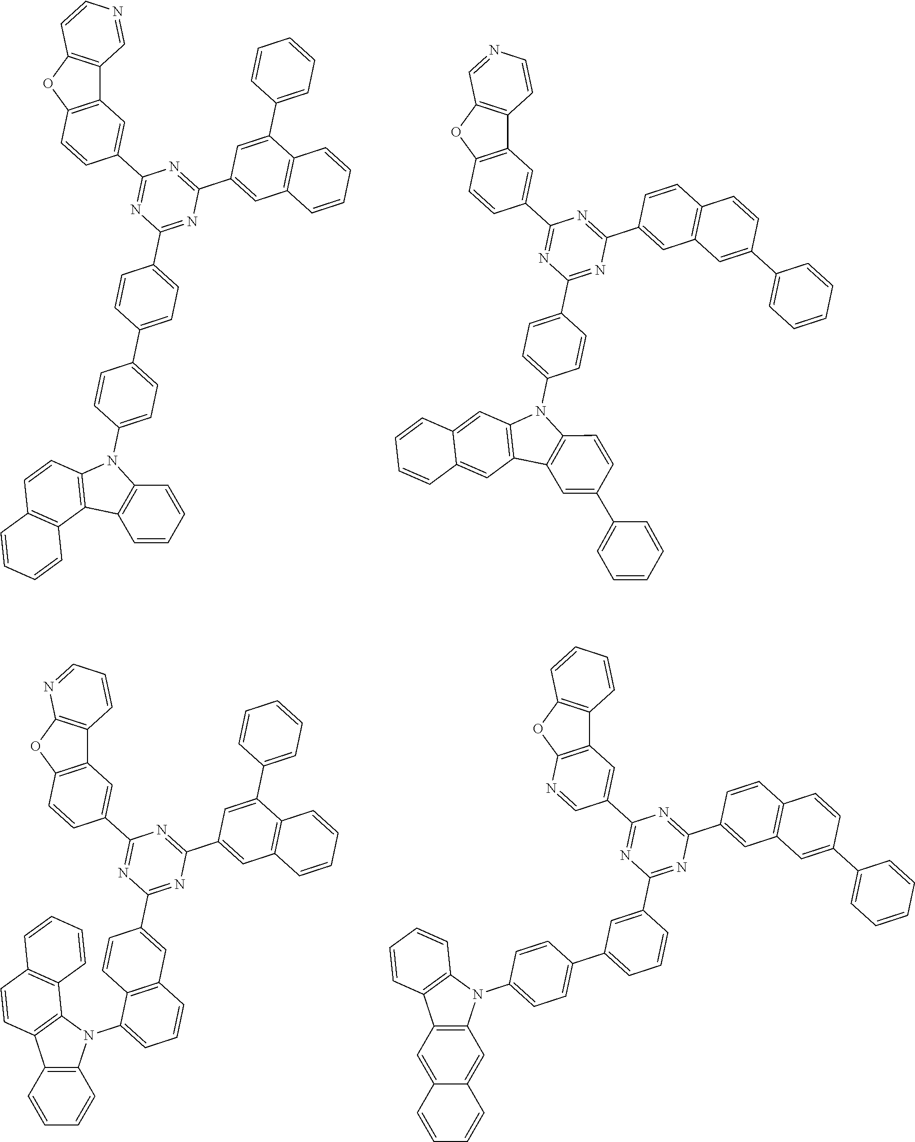 [SMILES: c1ccc(-c2cc(-c3nc(-c4ccc(-c5ccc(-n6c7ccccc7c7c8ccccc8ccc76)cc5)cc4)nc(-c4ccc5oc6ccncc6c5c4)n3)cc3ccccc23)cc1 ; c1ccc(-c2ccc3ccc(-c4nc(-c5ccc(-n6c7ccc(-c8ccccc8)cc7c7cc8ccccc8cc76)cc5)nc(-c5ccc6oc7cnccc7c6c5)n4)cc3c2)cc1 ; c1ccc(-c2cc(-c3nc(-c4ccc5c(-n6c7ccccc7c7ccc8ccccc8c76)cccc5c4)nc(-c4ccc5oc6ncccc6c5c4)n3)cc3ccccc23)cc1 ; c1ccc(-c2ccc3ccc(-c4nc(-c5cccc(-c6ccc(-n7c8ccccc8c8cc9ccccc9cc87)cc6)c5)nc(-c5cnc6oc7ccccc7c6c5)n4)cc3c2)cc1]

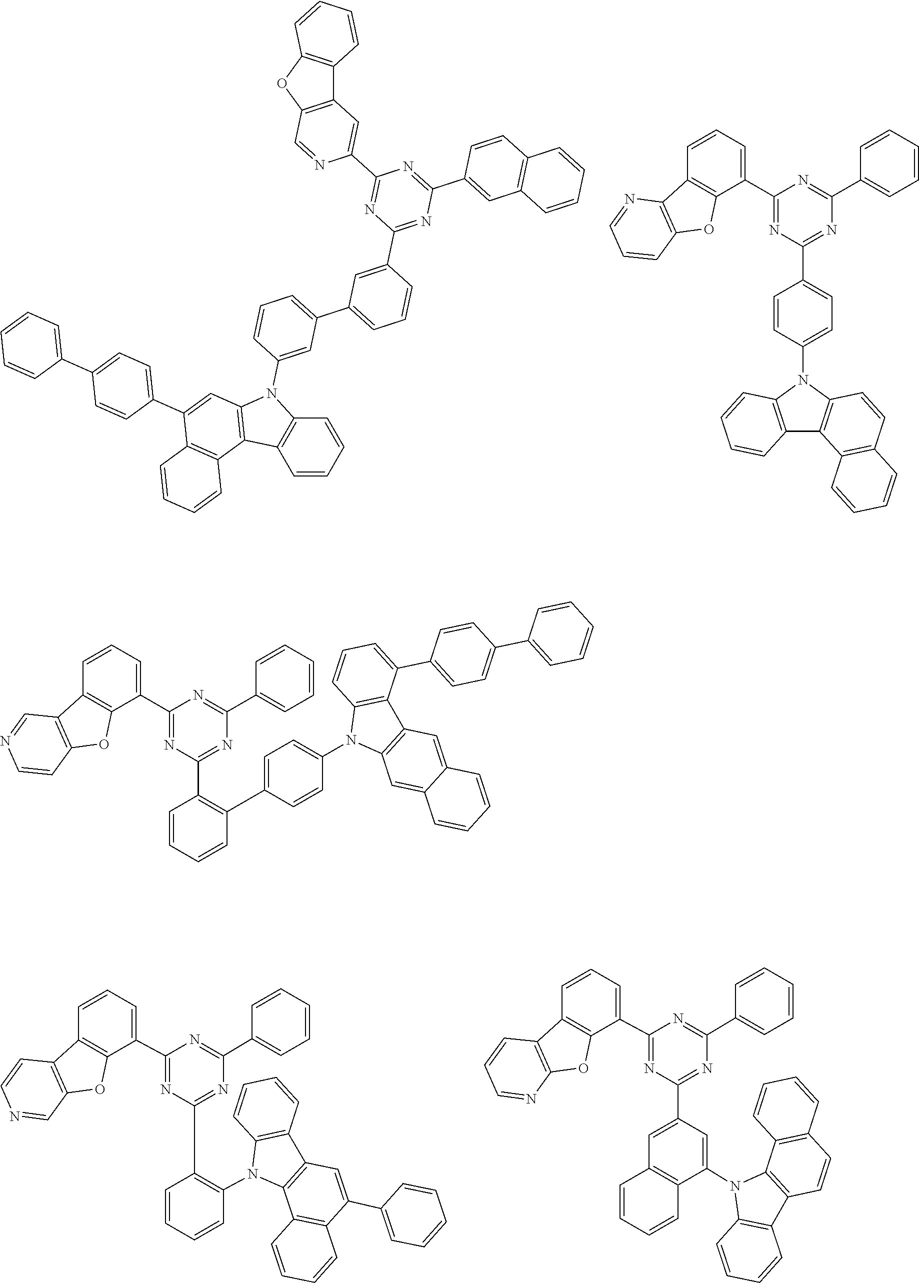

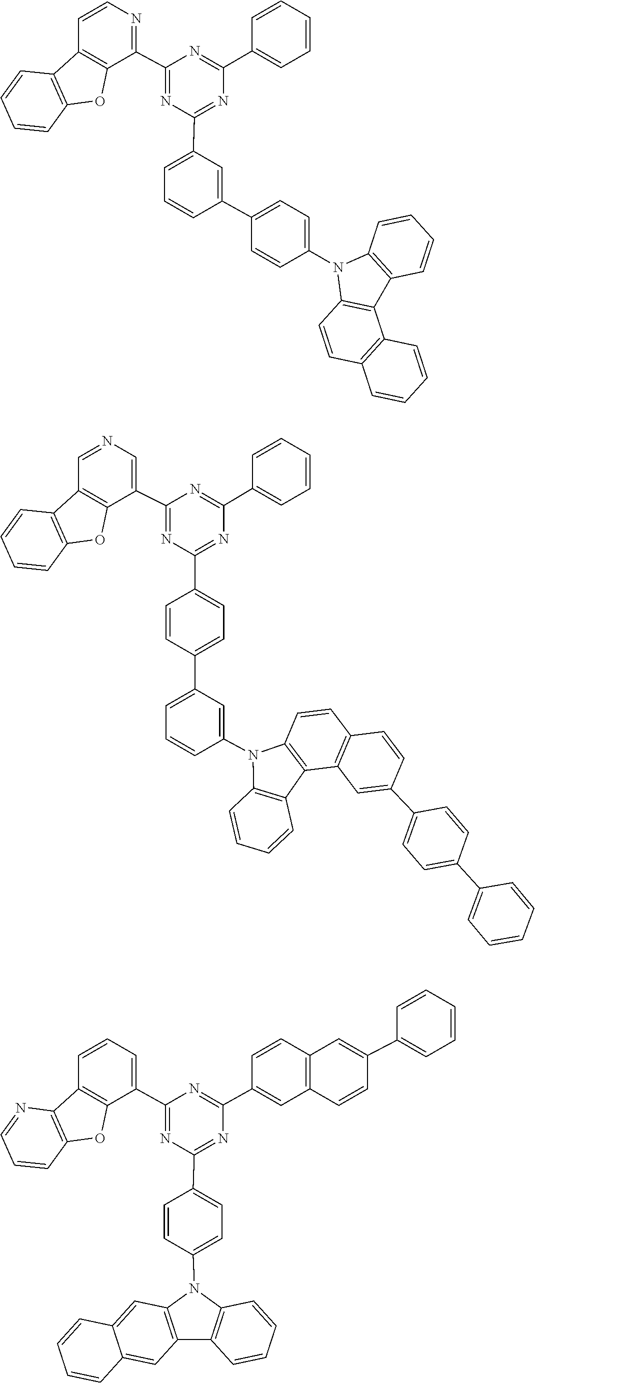 [SMILES: c1ccc(-c2nc(-c3cccc(-c4ccc(-n5c6ccccc6c6c7ccccc7ccc65)cc4)c3)nc(-c3nccc4c3oc3ccccc34)n2)cc1 ; c1ccc(-c2ccc(-c3ccc4ccc5c(c4c3)c3ccccc3n5-c3cccc(-c4ccc(-c5nc(-c6ccccc6)nc(-c6cncc7c6oc6ccccc67)n5)cc4)c3)cc2)cc1 ; c1ccc(-c2ccc3cc(-c4nc(-c5ccc(-n6c7ccccc7c7cc8ccccc8cc76)cc5)nc(-c5cccc6c5oc5cccnc56)n4)ccc3c2)cc1]

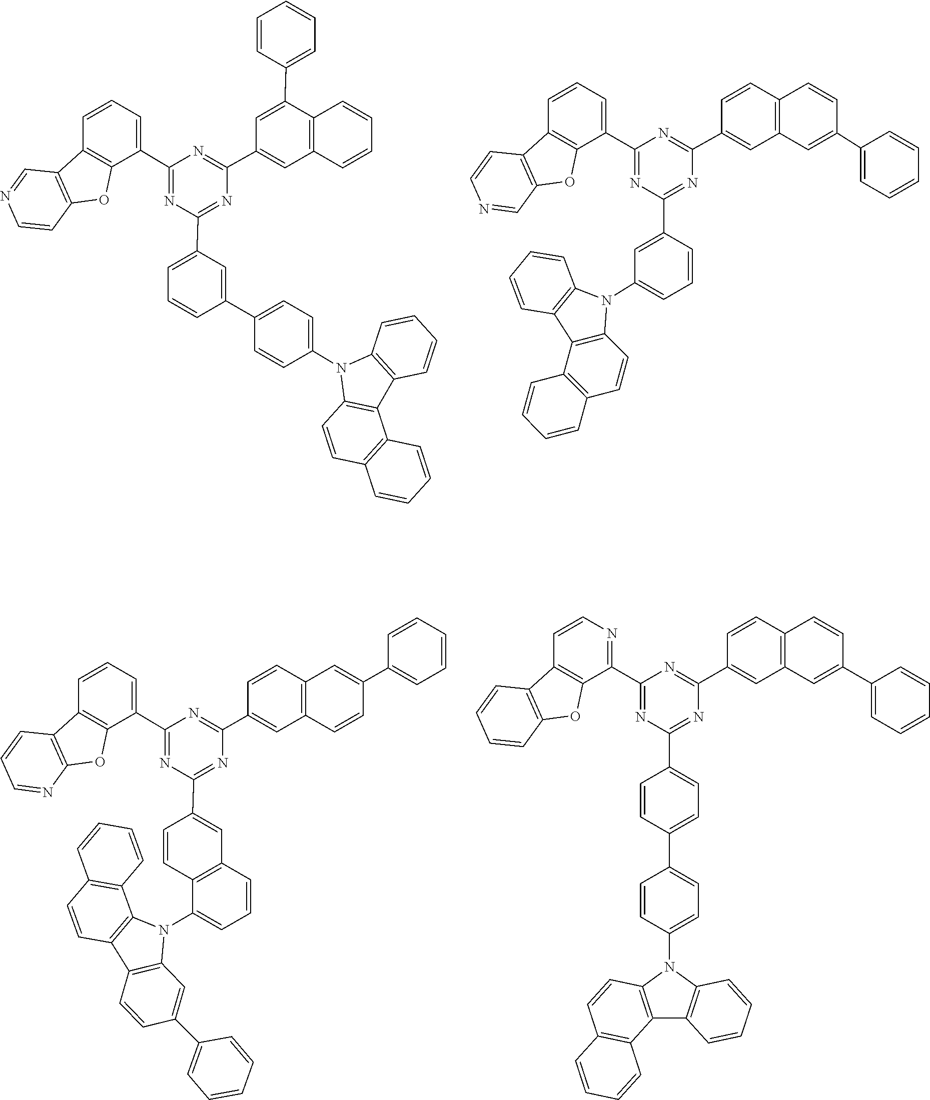 [SMILES: c1ccc(-c2cc(-c3nc(-c4cccc(-c5ccc(-n6c7ccccc7c7c8ccccc8ccc76)cc5)c4)nc(-c4cccc5c4oc4ccncc45)n3)cc3ccccc23)cc1 ; c1ccc(-c2ccc3ccc(-c4nc(-c5cccc(-n6c7ccccc7c7c8ccccc8ccc76)c5)nc(-c5cccc6c5oc5cnccc56)n4)cc3c2)cc1 ; c1ccc(-c2ccc3cc(-c4nc(-c5ccc6c(-n7c8cc(-c9ccccc9)ccc8c8ccc9ccccc9c87)cccc6c5)nc(-c5cccc6c5oc5ncccc56)n4)ccc3c2)cc1 ; c1ccc(-c2ccc3ccc(-c4nc(-c5ccc(-c6ccc(-n7c8ccccc8c8c9ccccc9ccc87)cc6)cc5)nc(-c5nccc6c5oc5ccccc56)n4)cc3c2)cc1]

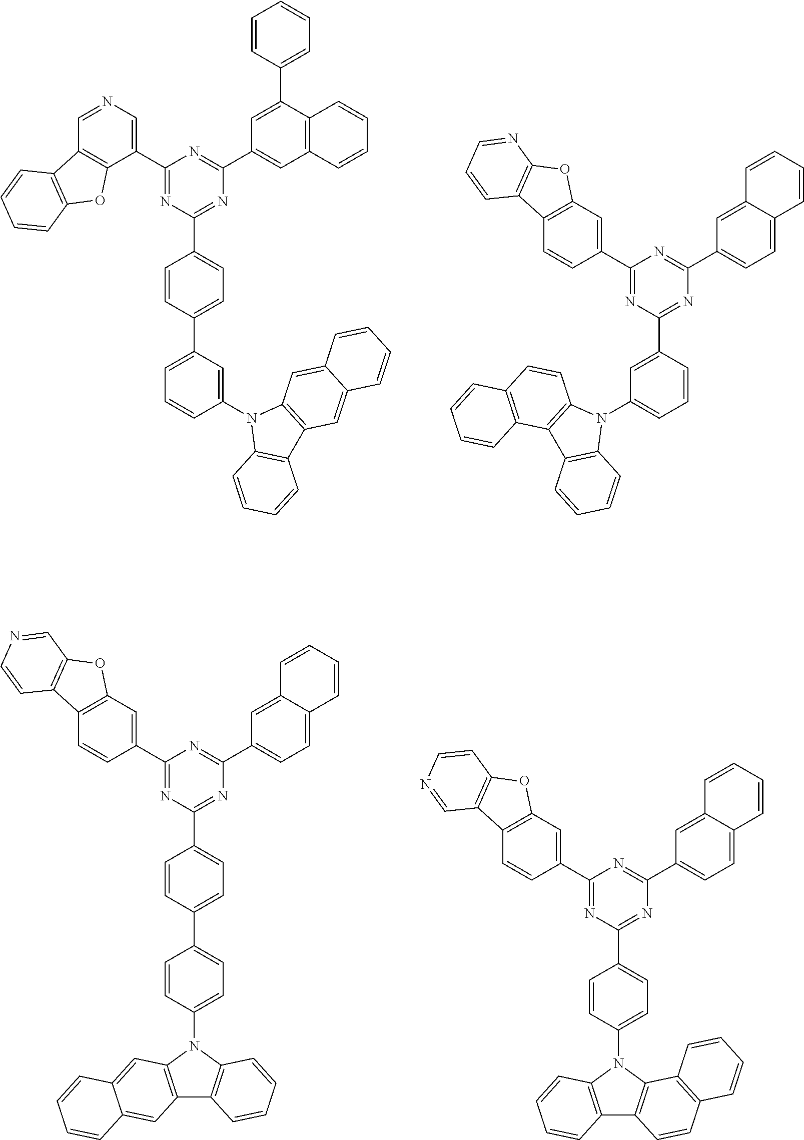 [SMILES: c1ccc(-c2cc(-c3nc(-c4ccc(-c5cccc(-n6c7ccccc7c7cc8ccccc8cc76)c5)cc4)nc(-c4cncc5c4oc4ccccc45)n3)cc3ccccc23)cc1 ; c1cc(-c2nc(-c3ccc4ccccc4c3)nc(-c3ccc4c(c3)oc3ncccc34)n2)cc(-n2c3ccccc3c3c4ccccc4ccc32)c1 ; c1ccc2cc(-c3nc(-c4ccc(-c5ccc(-n6c7ccccc7c7cc8ccccc8cc76)cc5)cc4)nc(-c4ccc5c(c4)oc4cnccc45)n3)ccc2c1 ; c1ccc2cc(-c3nc(-c4ccc(-n5c6ccccc6c6ccc7ccccc7c65)cc4)nc(-c4ccc5c(c4)oc4ccncc45)n3)ccc2c1]

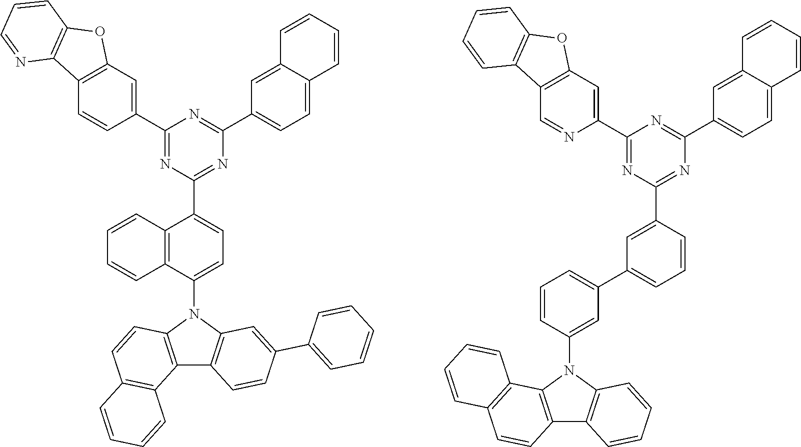 [SMILES: c1ccc(-c2ccc3c4c5ccccc5ccc4n(-c4ccc(-c5nc(-c6ccc7ccccc7c6)nc(-c6ccc7c(c6)oc6cccnc67)n5)c5ccccc45)c3c2)cc1 ; c1cc(-c2cccc(-n3c4ccccc4c4ccc5ccccc5c43)c2)cc(-c2nc(-c3ccc4ccccc4c3)nc(-c3cc4oc5ccccc5c4cn3)n2)c1]

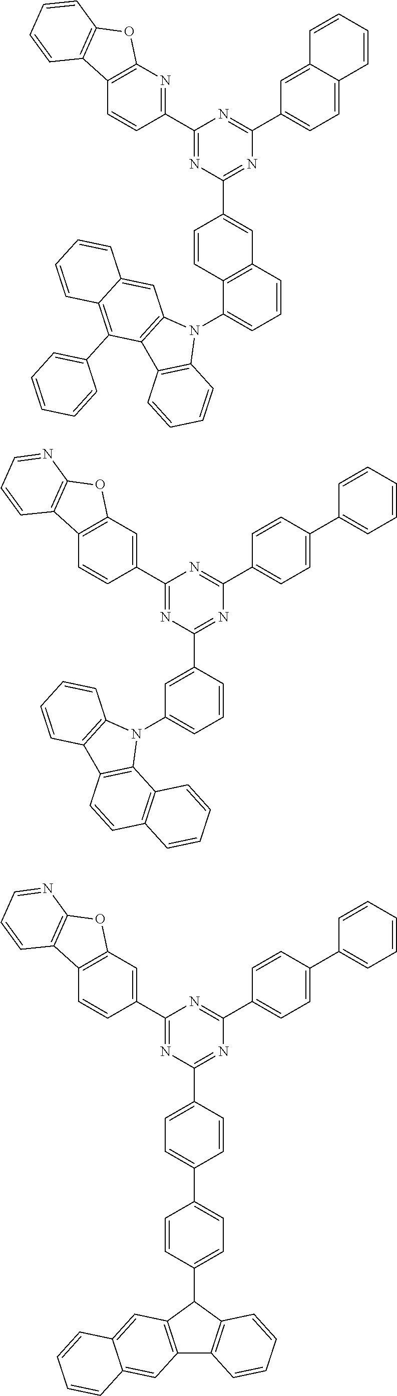

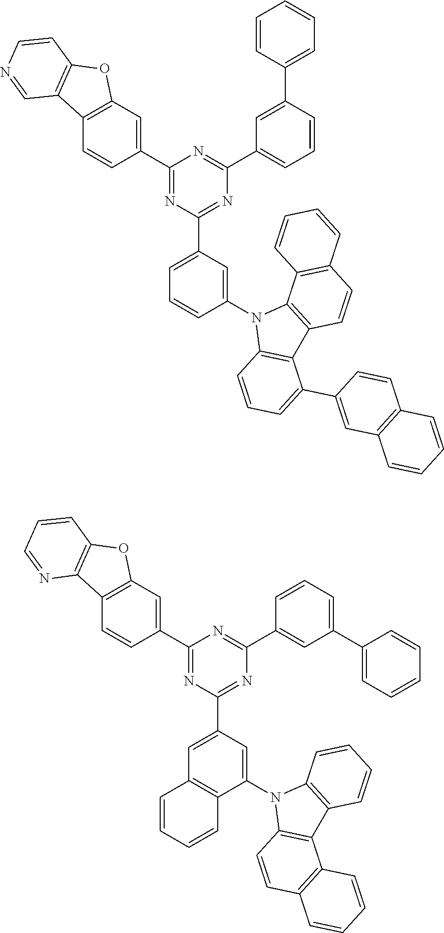 [SMILES: c1ccc(-c2cccc(-c3nc(-c4cccc(-n5c6cccc(-c7ccc8ccccc8c7)c6c6ccc7ccccc7c65)c4)nc(-c4ccc5c(c4)oc4ccncc45)n3)c2)cc1 ; c1ccc(-c2cccc(-c3nc(-c4cc(-n5c6ccccc6c6c7ccccc7ccc65)c5ccccc5c4)nc(-c4ccc5c(c4)oc4cccnc45)n3)c2)cc1]

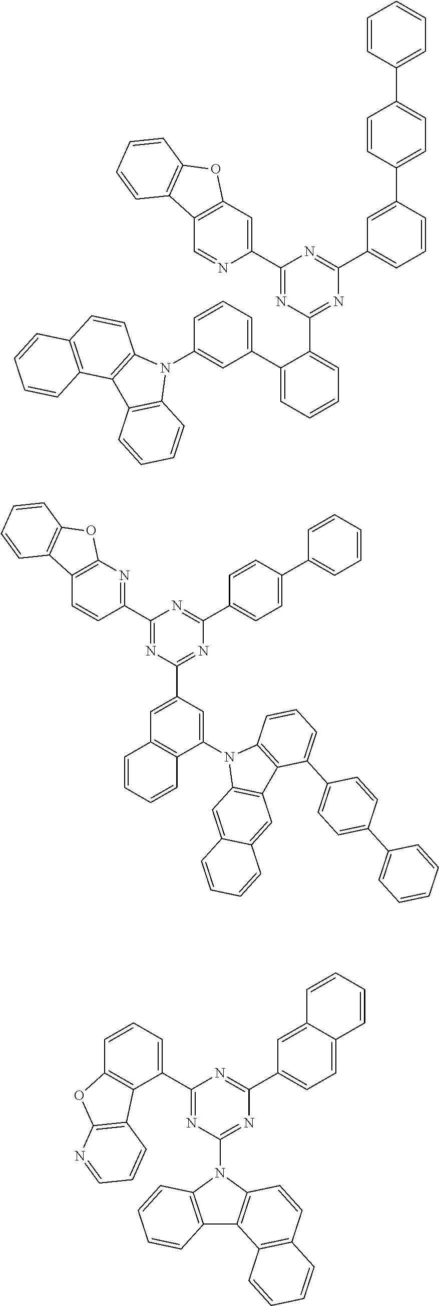 [SMILES: c1ccc(-c2ccc(-c3cccc(-c4nc(-c5cc6oc7ccccc7c6cn5)nc(-c5ccccc5-c5cccc(-n6c7ccccc7c7c8ccccc8ccc76)c5)n4)c3)cc2)cc1 ; c1ccc(-c2ccc(-c3nc(-c4cc(-n5c6cc7ccccc7cc6c6c(-c7ccc(-c8ccccc8)cc7)cccc65)c5ccccc5c4)nc(-c4ccc5c(n4)oc4ccccc45)n3)cc2)cc1 ; c1ccc2cc(-c3nc(-c4cccc5oc6ncccc6c45)nc(-n4c5ccccc5c5c6ccccc6ccc54)n3)ccc2c1]

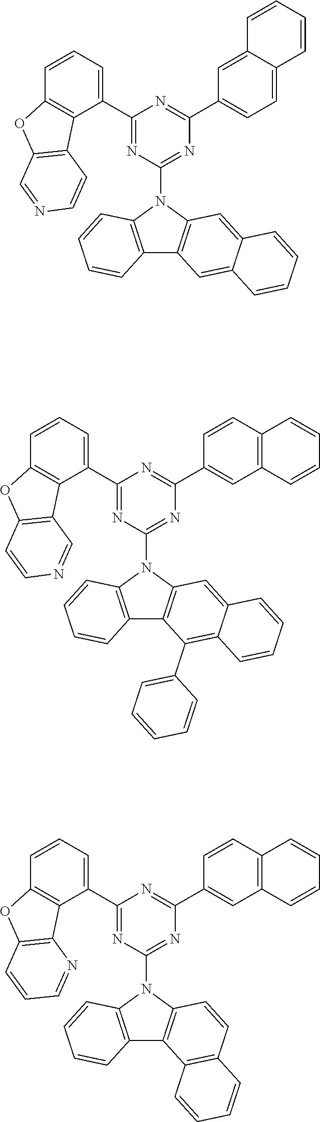 [SMILES: c1ccc2cc(-c3nc(-c4cccc5oc6cnccc6c45)nc(-n4c5ccccc5c5cc6ccccc6cc54)n3)ccc2c1 ; c1ccc(-c2c3ccccc3cc3c2c2ccccc2n3-c2nc(-c3ccc4ccccc4c3)nc(-c3cccc4oc5ccncc5c34)n2)cc1 ; c1ccc2cc(-c3nc(-c4cccc5oc6cccnc6c45)nc(-n4c5ccccc5c5c6ccccc6ccc54)n3)ccc2c1]

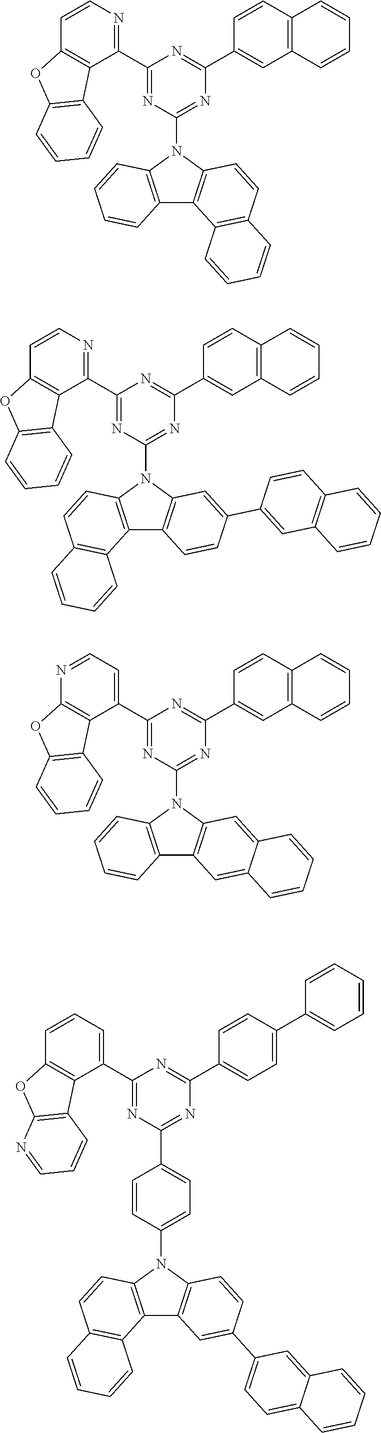

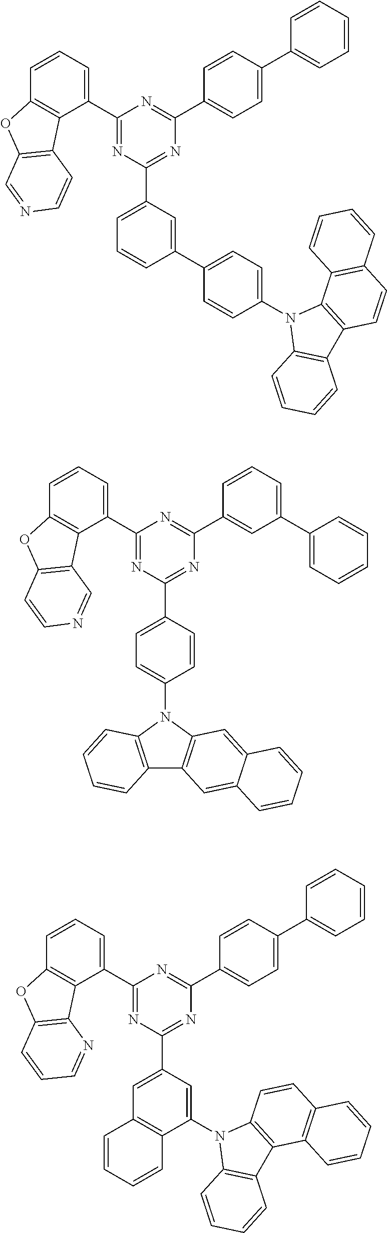 [SMILES: c1ccc(-c2ccc(-c3nc(-c4cccc(-c5ccc(-n6c7ccccc7c7ccc8ccccc8c76)cc5)c4)nc(-c4cccc5oc6cnccc6c45)n3)cc2)cc1 ; c1ccc(-c2cccc(-c3nc(-c4ccc(-n5c6ccccc6c6cc7ccccc7cc65)cc4)nc(-c4cccc5oc6ccncc6c45)n3)c2)cc1 ; c1ccc(-c2ccc(-c3nc(-c4cc(-n5c6ccccc6c6c7ccccc7ccc65)c5ccccc5c4)nc(-c4cccc5oc6cccnc6c45)n3)cc2)cc1]

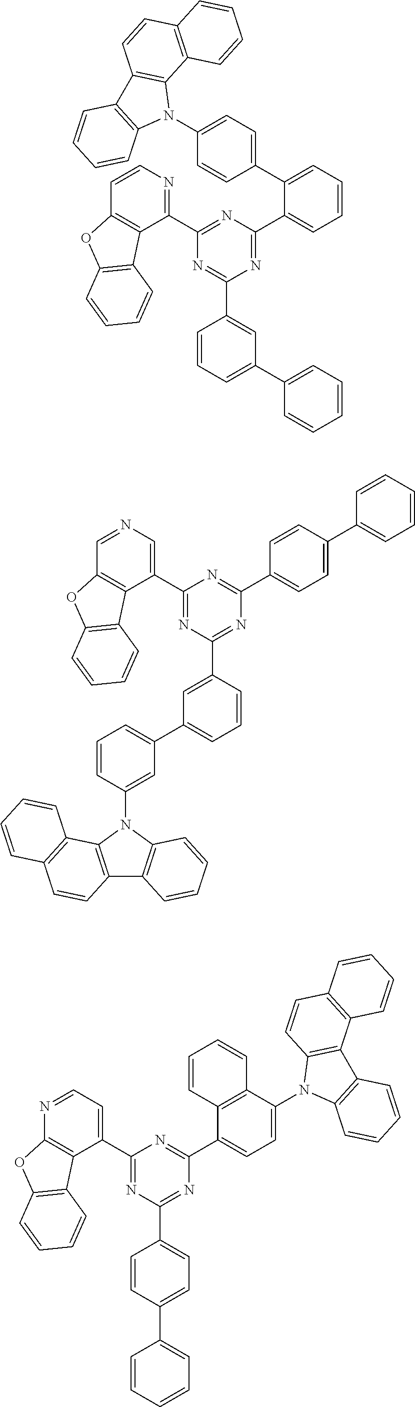 [SMILES: c1ccc(-c2cccc(-c3nc(-c4ccccc4-c4ccc(-n5c6ccccc6c6ccc7ccccc7c65)cc4)nc(-c4nccc5oc6ccccc6c45)n3)c2)cc1 ; c1ccc(-c2ccc(-c3nc(-c4cccc(-c5cccc(-n6c7ccccc7c7ccc8ccccc8c76)c5)c4)nc(-c4cncc5oc6ccccc6c45)n3)cc2)cc1 ; c1ccc(-c2ccc(-c3nc(-c4ccc(-n5c6ccccc6c6c7ccccc7ccc65)c5ccccc45)nc(-c4ccnc5oc6ccccc6c45)n3)cc2)cc1]

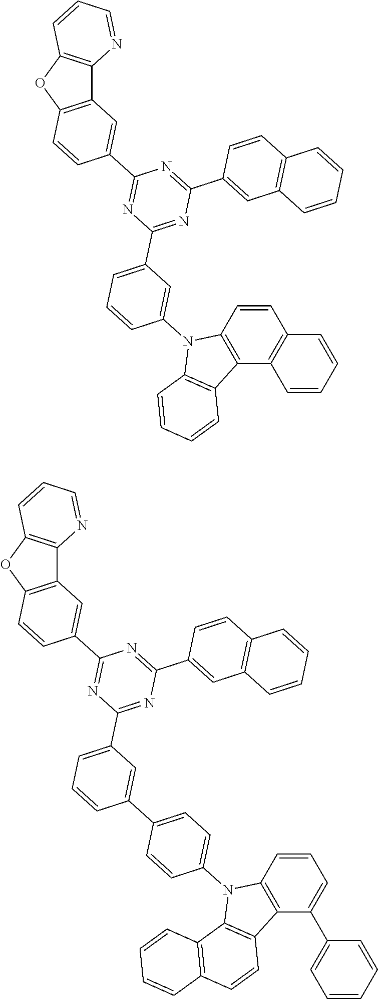 [SMILES: c1cc(-c2nc(-c3ccc4ccccc4c3)nc(-c3ccc4oc5cccnc5c4c3)n2)cc(-n2c3ccccc3c3c4ccccc4ccc32)c1 ; c1ccc(-c2cccc3c2c2ccc4ccccc4c2n3-c2ccc(-c3cccc(-c4nc(-c5ccc6ccccc6c5)nc(-c5ccc6oc7cccnc7c6c5)n4)c3)cc2)cc1]

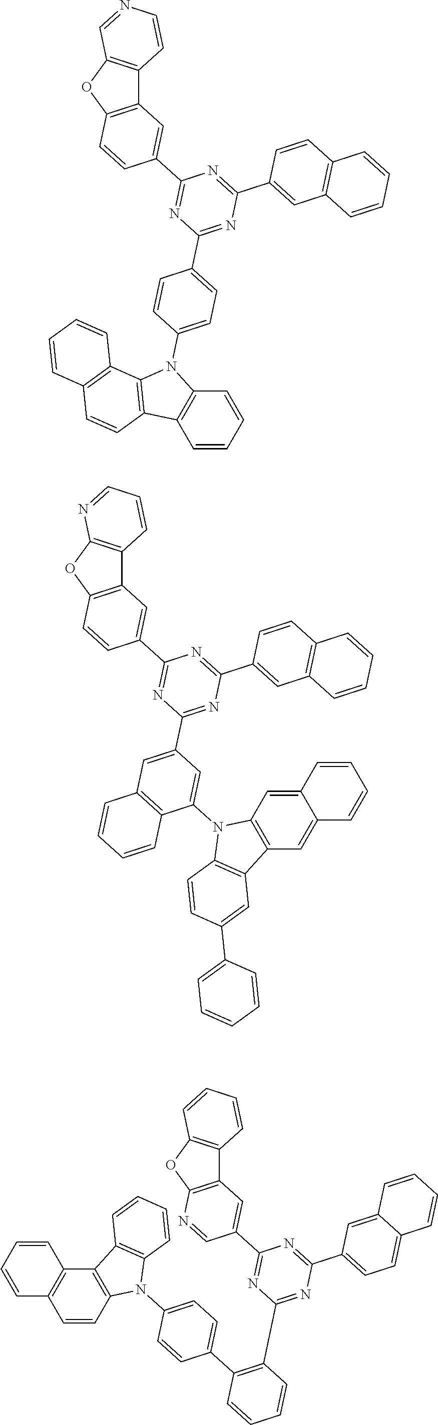 [SMILES: c1ccc2cc(-c3nc(-c4ccc(-n5c6ccccc6c6ccc7ccccc7c65)cc4)nc(-c4ccc5oc6cnccc6c5c4)n3)ccc2c1 ; c1ccc(-c2ccc3c(c2)c2cc4ccccc4cc2n3-c2cc(-c3nc(-c4ccc5ccccc5c4)nc(-c4ccc5oc6ncccc6c5c4)n3)cc3ccccc23)cc1 ; c1ccc(-c2nc(-c3ccc4ccccc4c3)nc(-c3cnc4oc5ccccc5c4c3)n2)c(-c2ccc(-n3c4ccccc4c4c5ccccc5ccc43)cc2)c1]

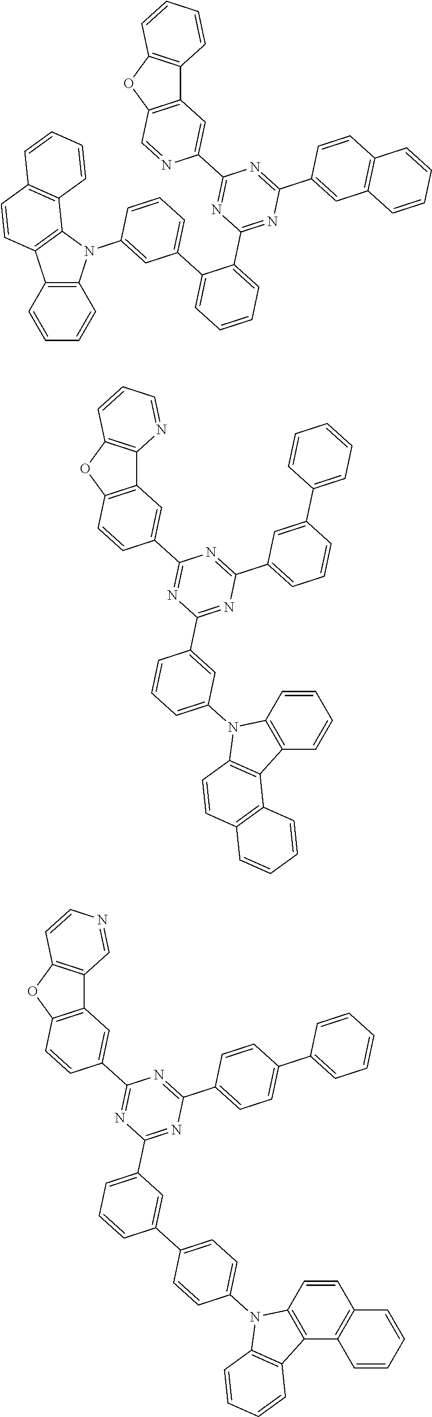 [SMILES: c1cc(-c2ccccc2-c2nc(-c3ccc4ccccc4c3)nc(-c3cc4c(cn3)oc3ccccc34)n2)cc(-n2c3ccccc3c3ccc4ccccc4c32)c1 ; c1ccc(-c2cccc(-c3nc(-c4cccc(-n5c6ccccc6c6c7ccccc7ccc65)c4)nc(-c4ccc5oc6cccnc6c5c4)n3)c2)cc1 ; c1ccc(-c2ccc(-c3nc(-c4cccc(-c5ccc(-n6c7ccccc7c7c8ccccc8ccc76)cc5)c4)nc(-c4ccc5oc6ccncc6c5c4)n3)cc2)cc1]

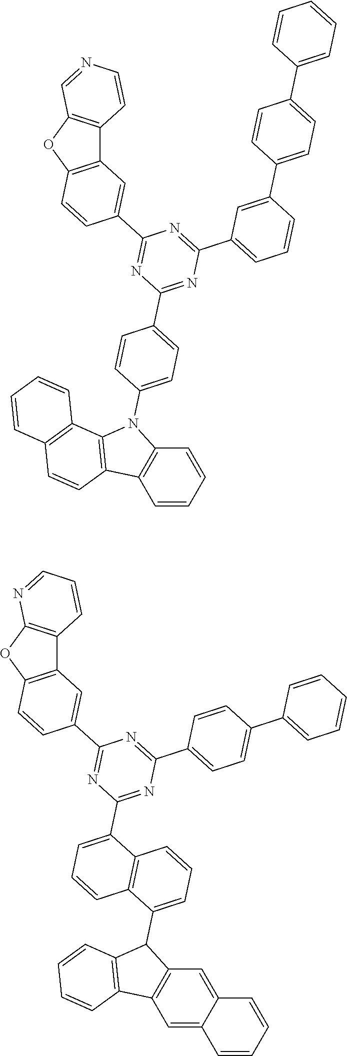 [SMILES: c1ccc(-c2ccc(-c3cccc(-c4nc(-c5ccc(-n6c7ccccc7c7ccc8ccccc8c76)cc5)nc(-c5ccc6oc7cnccc7c6c5)n4)c3)cc2)cc1 ; c1ccc(-c2ccc(-c3nc(-c4ccc5oc6ncccc6c5c4)nc(-c4cccc5c(C6c7ccccc7-c7cc8ccccc8cc76)cccc45)n3)cc2)cc1]

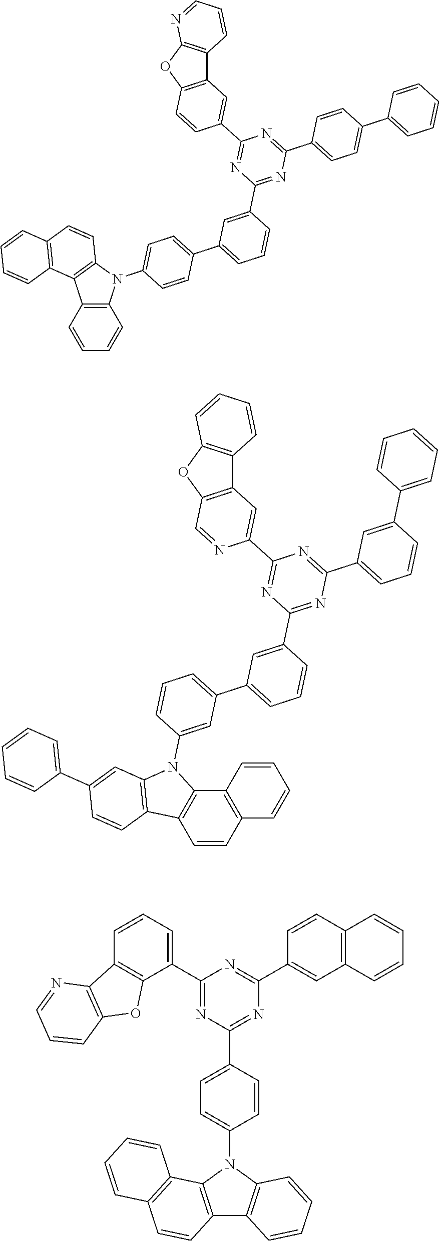 [SMILES: c1ccc(-c2ccc(-c3nc(-c4cccc(-c5ccc(-n6c7ccccc7c7c8ccccc8ccc76)cc5)c4)nc(-c4ccc5oc6ncccc6c5c4)n3)cc2)cc1 ; c1ccc(-c2cccc(-c3nc(-c4cccc(-c5cccc(-n6c7cc(-c8ccccc8)ccc7c7ccc8ccccc8c76)c5)c4)nc(-c4cc5c(cn4)oc4ccccc45)n3)c2)cc1 ; c1ccc2cc(-c3nc(-c4ccc(-n5c6ccccc6c6ccc7ccccc7c65)cc4)nc(-c4cccc5c4oc4cccnc45)n3)ccc2c1]

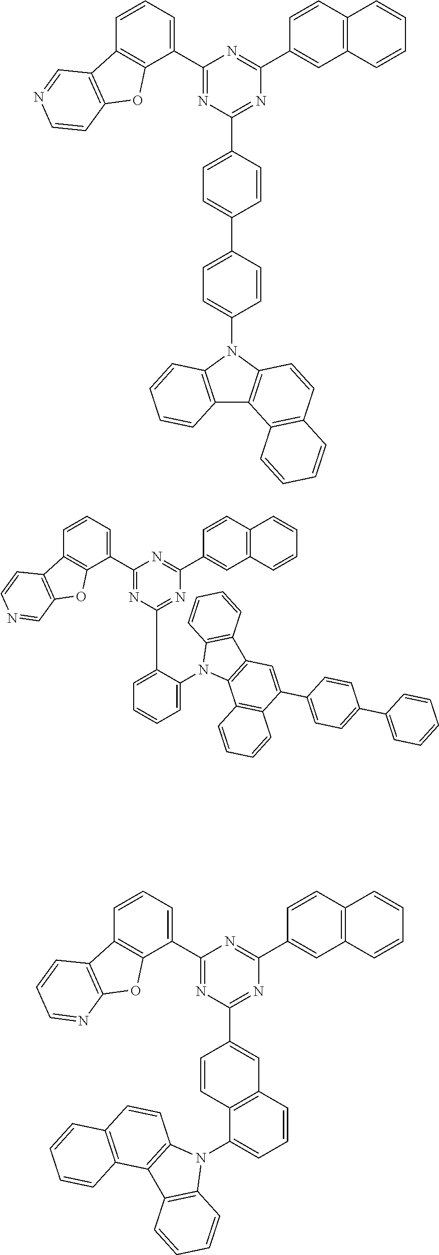

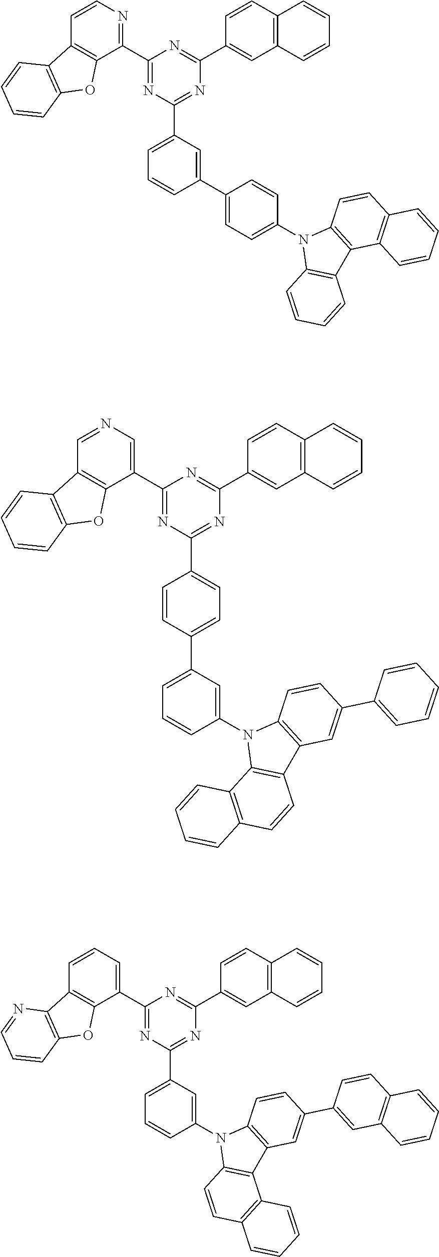 [SMILES: c1cc(-c2ccc(-n3c4ccccc4c4c5ccccc5ccc43)cc2)cc(-c2nc(-c3ccc4ccccc4c3)nc(-c3nccc4c3oc3ccccc34)n2)c1 ; c1ccc(-c2ccc3c(c2)c2ccc4ccccc4c2n3-c2cccc(-c3ccc(-c4nc(-c5ccc6ccccc6c5)nc(-c5cncc6c5oc5ccccc56)n4)cc3)c2)cc1 ; c1cc(-c2nc(-c3ccc4ccccc4c3)nc(-c3cccc4c3oc3cccnc34)n2)cc(-n2c3ccc(-c4ccc5ccccc5c4)cc3c3c4ccccc4ccc32)c1]

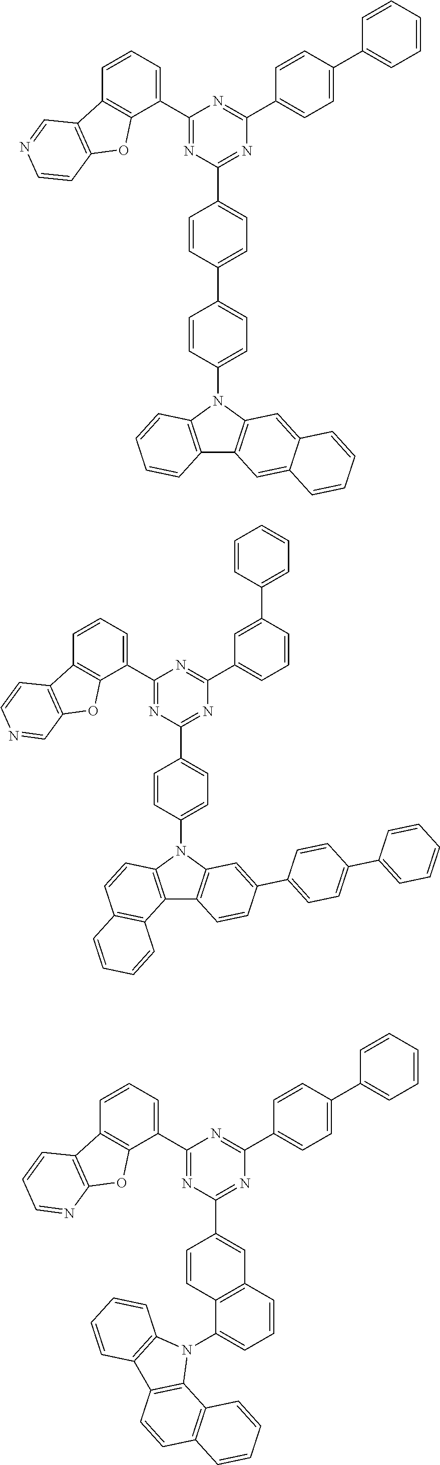 [SMILES: c1ccc(-c2ccc(-c3nc(-c4ccc(-c5ccc(-n6c7ccccc7c7cc8ccccc8cc76)cc5)cc4)nc(-c4cccc5c4oc4ccncc45)n3)cc2)cc1 ; c1ccc(-c2ccc(-c3ccc4c5c6ccccc6ccc5n(-c5ccc(-c6nc(-c7cccc(-c8ccccc8)c7)nc(-c7cccc8c7oc7cnccc78)n6)cc5)c4c3)cc2)cc1 ; c1ccc(-c2ccc(-c3nc(-c4ccc5c(-n6c7ccccc7c7ccc8ccccc8c76)cccc5c4)nc(-c4cccc5c4oc4ncccc45)n3)cc2)cc1]

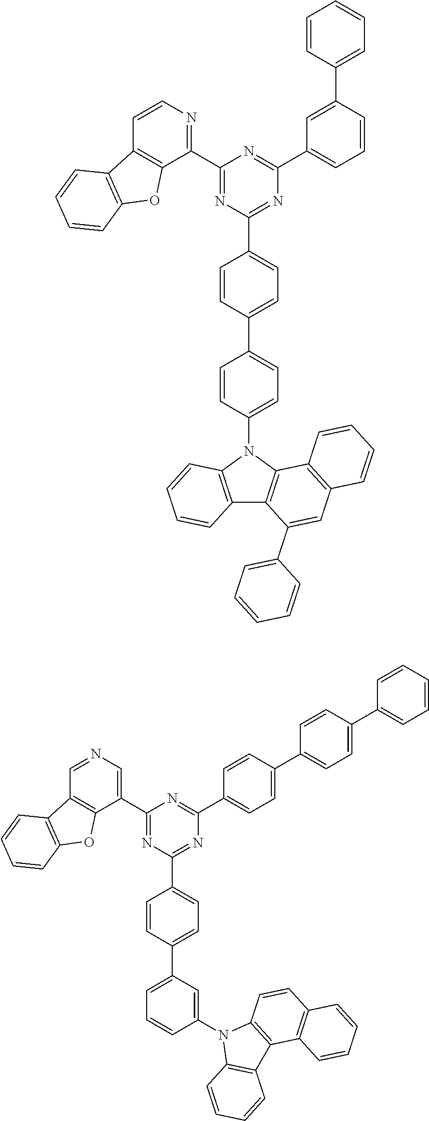 [SMILES: c1ccc(-c2cccc(-c3nc(-c4ccc(-c5ccc(-n6c7ccccc7c7c(-c8ccccc8)cc8ccccc8c76)cc5)cc4)nc(-c4nccc5c4oc4ccccc45)n3)c2)cc1 ; c1ccc(-c2ccc(-c3ccc(-c4nc(-c5ccc(-c6cccc(-n7c8ccccc8c8c9ccccc9ccc87)c6)cc5)nc(-c5cncc6c5oc5ccccc56)n4)cc3)cc2)cc1]

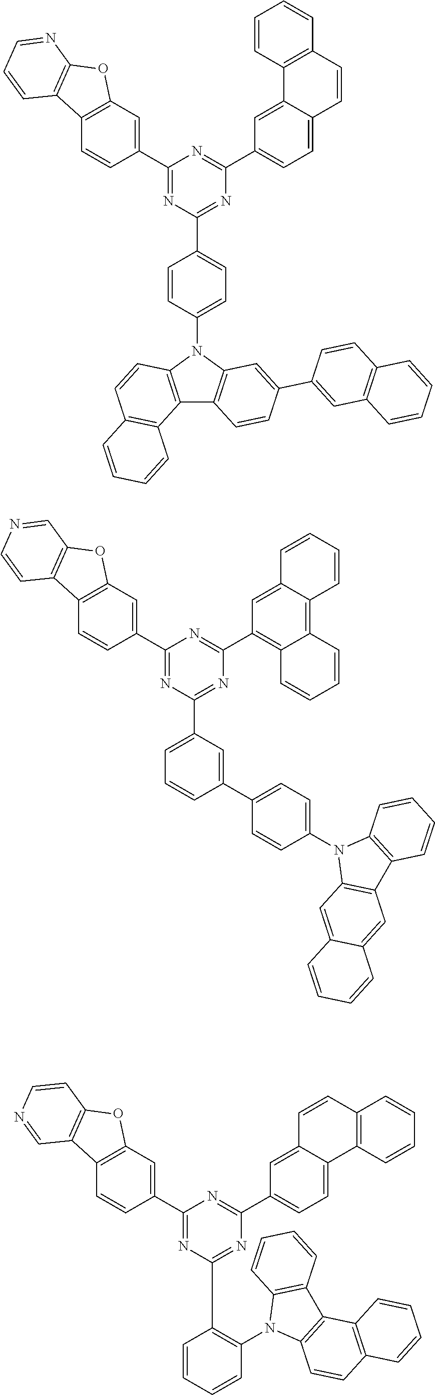 [SMILES: c1ccc2cc(-c3ccc4c5c6ccccc6ccc5n(-c5ccc(-c6nc(-c7ccc8c(c7)oc7ncccc78)nc(-c7ccc8ccc9ccccc9c8c7)n6)cc5)c4c3)ccc2c1 ; c1cc(-c2ccc(-n3c4ccccc4c4cc5ccccc5cc43)cc2)cc(-c2nc(-c3ccc4c(c3)oc3cnccc34)nc(-c3cc4ccccc4c4ccccc34)n2)c1 ; c1ccc(-n2c3ccccc3c3c4ccccc4ccc32)c(-c2nc(-c3ccc4c(ccc5ccccc54)c3)nc(-c3ccc4c(c3)oc3ccncc34)n2)c1]

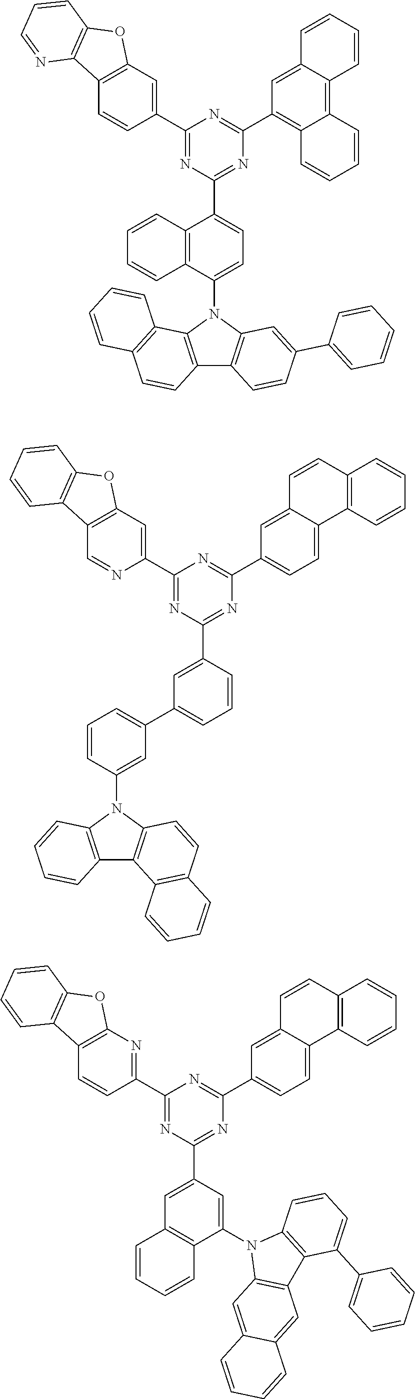 [SMILES: c1ccc(-c2ccc3c4ccc5ccccc5c4n(-c4ccc(-c5nc(-c6ccc7c(c6)oc6cccnc67)nc(-c6cc7ccccc7c7ccccc67)n5)c5ccccc45)c3c2)cc1 ; c1cc(-c2cccc(-n3c4ccccc4c4c5ccccc5ccc43)c2)cc(-c2nc(-c3ccc4c(ccc5ccccc54)c3)nc(-c3cc4oc5ccccc5c4cn3)n2)c1 ; c1ccc(-c2cccc3c2c2cc4ccccc4cc2n3-c2cc(-c3nc(-c4ccc5c(ccc6ccccc65)c4)nc(-c4ccc5c(n4)oc4ccccc45)n3)cc3ccccc23)cc1]

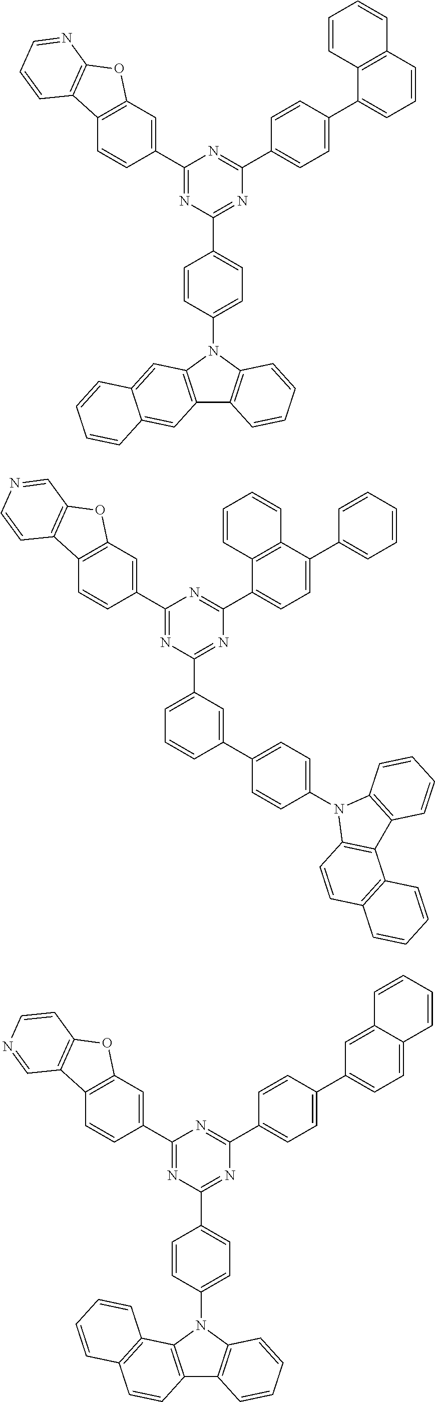

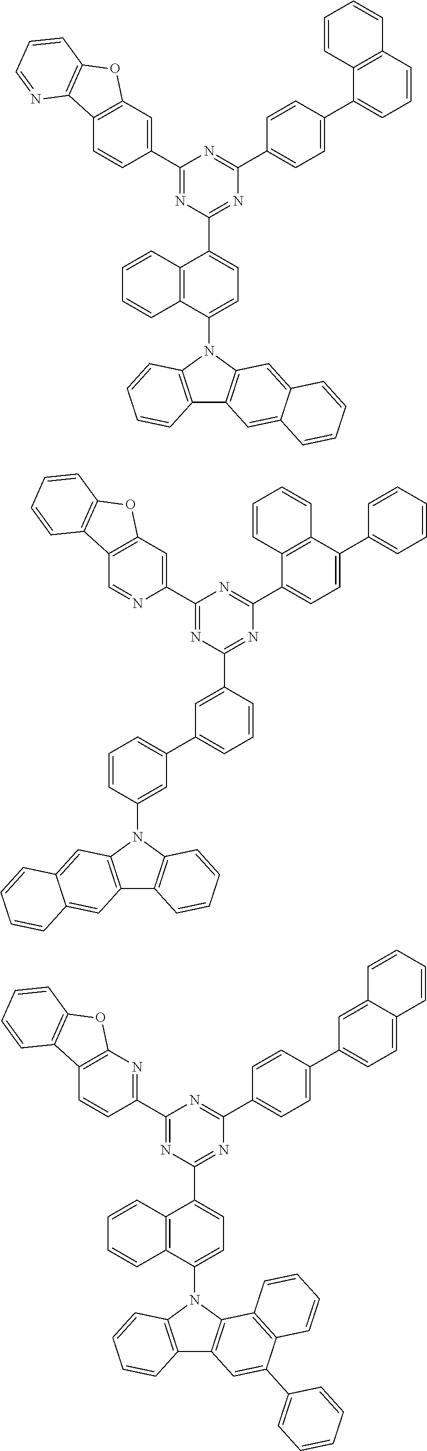 [SMILES: c1ccc2cc3c(cc2c1)c1ccccc1n3-c1ccc(-c2nc(-c3ccc(-c4cccc5ccccc45)cc3)nc(-c3ccc4c(c3)oc3cccnc34)n2)c2ccccc12 ; c1ccc(-c2ccc(-c3nc(-c4cccc(-c5cccc(-n6c7ccccc7c7cc8ccccc8cc76)c5)c4)nc(-c4cc5oc6ccccc6c5cn4)n3)c3ccccc23)cc1 ; c1ccc(-c2cc3c4ccccc4n(-c4ccc(-c5nc(-c6ccc(-c7ccc8ccccc8c7)cc6)nc(-c6ccc7c(n6)oc6ccccc67)n5)c5ccccc45)c3c3ccccc23)cc1]

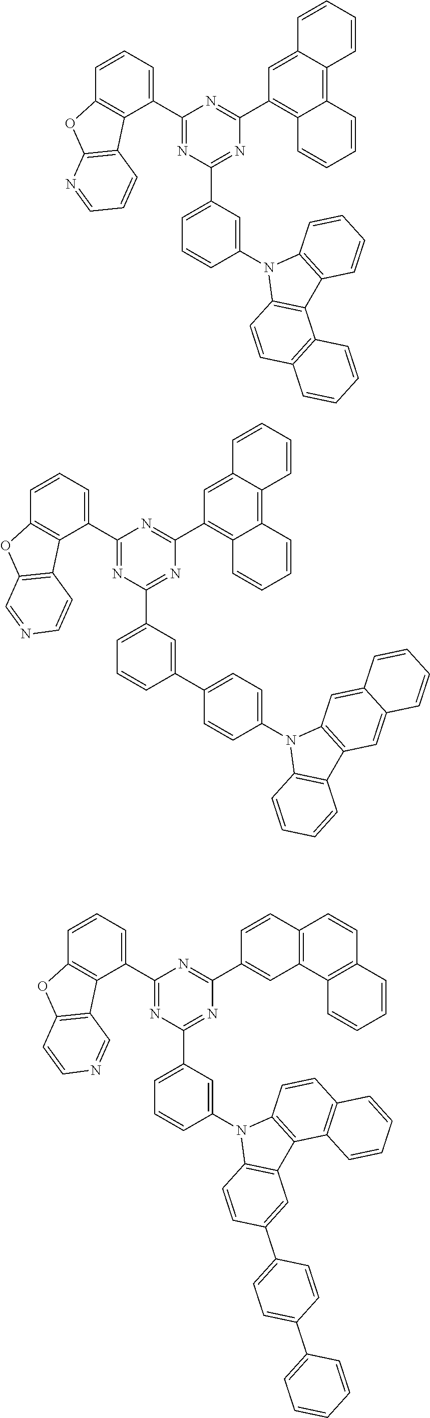

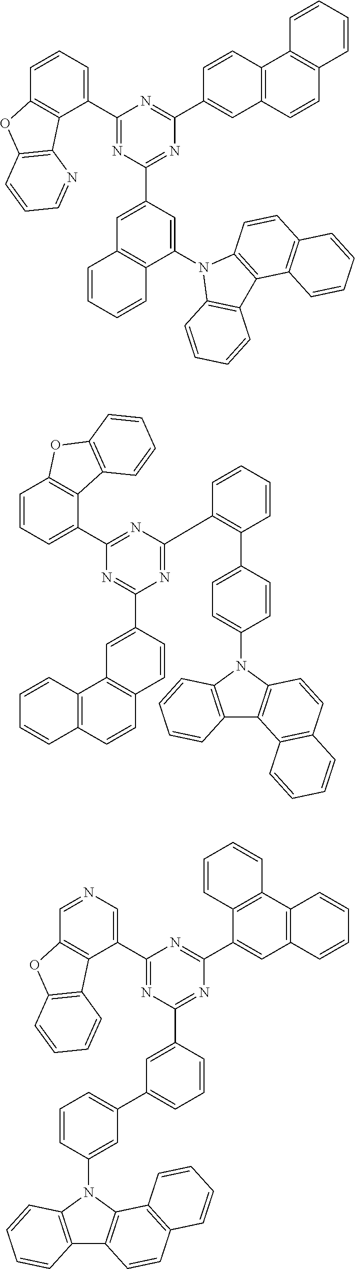 [SMILES: c1ccc2c(-n3c4ccccc4c4c5ccccc5ccc43)cc(-c3nc(-c4ccc5c(ccc6ccccc65)c4)nc(-c4cccc5oc6cccnc6c45)n3)cc2c1 ; c1ccc(-c2nc(-c3ccc4ccc5ccccc5c4c3)nc(-c3cccc4oc5ccccc5c34)n2)c(-c2ccc(-n3c4ccccc4c4c5ccccc5ccc43)cc2)c1 ; c1cc(-c2cccc(-n3c4ccccc4c4ccc5ccccc5c43)c2)cc(-c2nc(-c3cc4ccccc4c4ccccc34)nc(-c3cncc4oc5ccccc5c34)n2)c1]

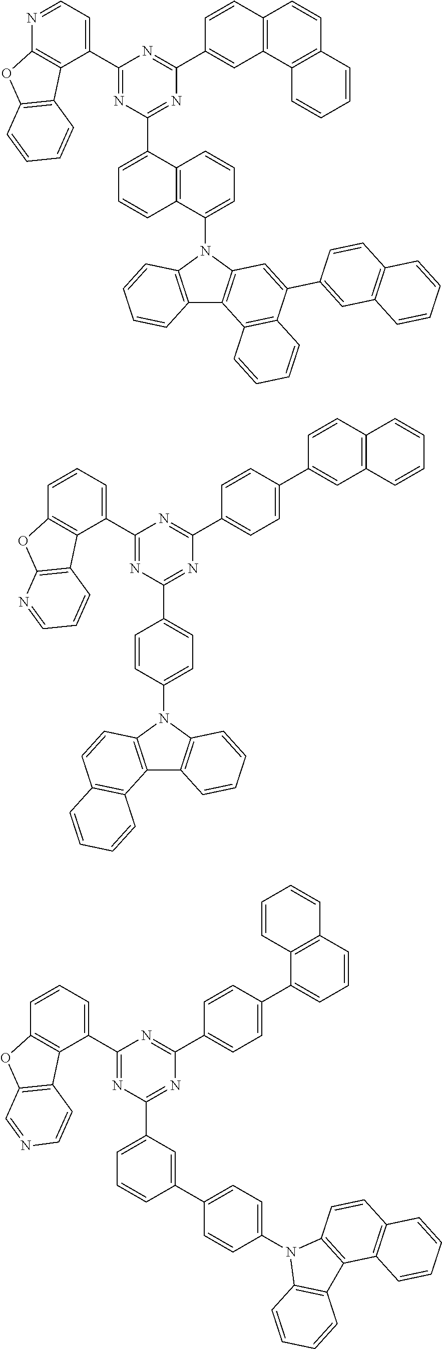 [SMILES: c1ccc2cc(-c3cc4c(c5ccccc35)c3ccccc3n4-c3cccc4c(-c5nc(-c6ccc7ccc8ccccc8c7c6)nc(-c6ccnc7oc8ccccc8c67)n5)cccc34)ccc2c1 ; c1ccc2cc(-c3ccc(-c4nc(-c5ccc(-n6c7ccccc7c7c8ccccc8ccc76)cc5)nc(-c5cccc6oc7ncccc7c56)n4)cc3)ccc2c1 ; c1cc(-c2ccc(-n3c4ccccc4c4c5ccccc5ccc43)cc2)cc(-c2nc(-c3ccc(-c4cccc5ccccc45)cc3)nc(-c3cccc4oc5cnccc5c34)n2)c1]

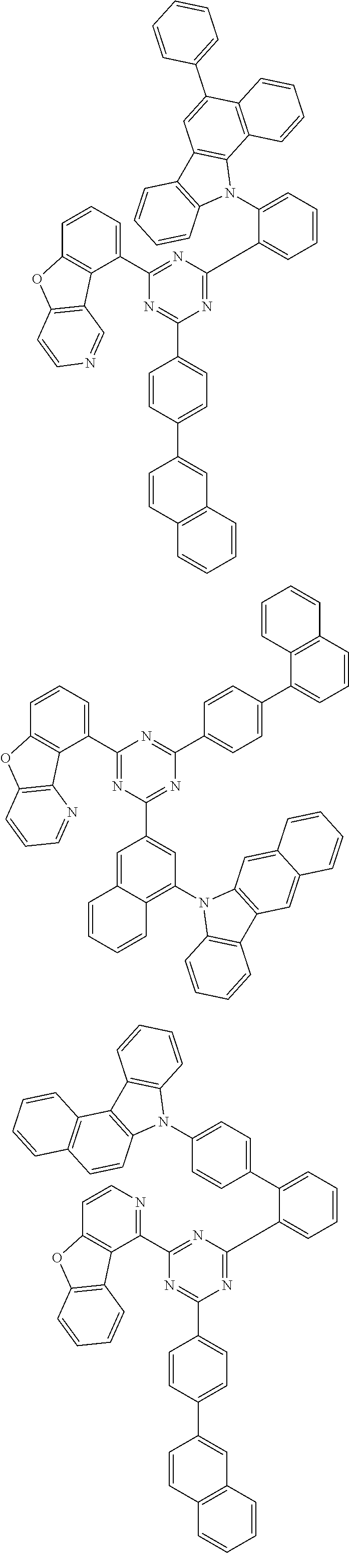

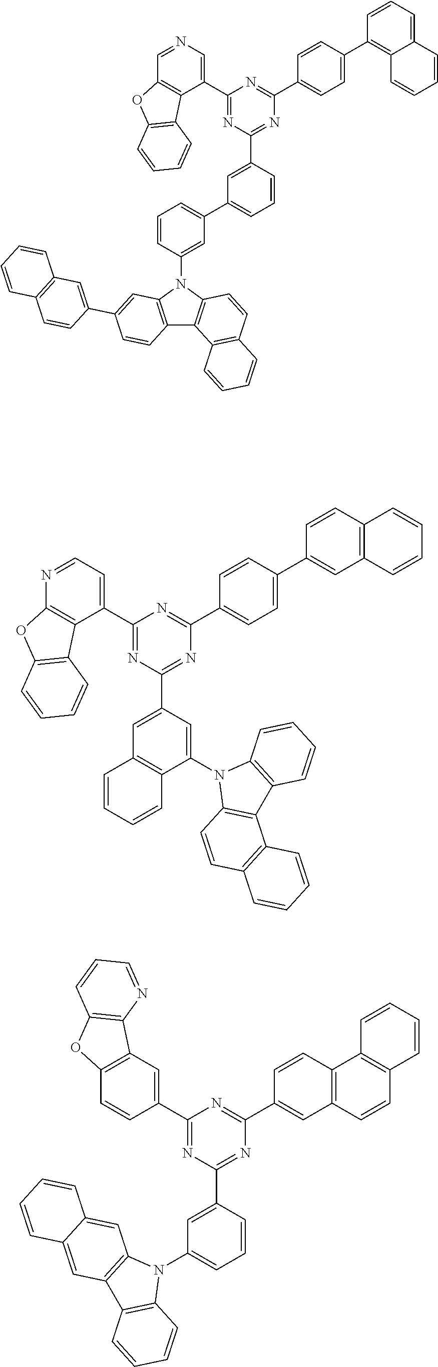 [SMILES: c1cc(-c2cccc(-n3c4cc(-c5ccc6ccccc6c5)ccc4c4c5ccccc5ccc43)c2)cc(-c2nc(-c3ccc(-c4cccc5ccccc45)cc3)nc(-c3cncc4oc5ccccc5c34)n2)c1 ; c1ccc2cc(-c3ccc(-c4nc(-c5cc(-n6c7ccccc7c7c8ccccc8ccc76)c6ccccc6c5)nc(-c5ccnc6oc7ccccc7c56)n4)cc3)ccc2c1 ; c1cc(-c2nc(-c3ccc4c(ccc5ccccc54)c3)nc(-c3ccc4oc5cccnc5c4c3)n2)cc(-n2c3ccccc3c3cc4ccccc4cc32)c1]

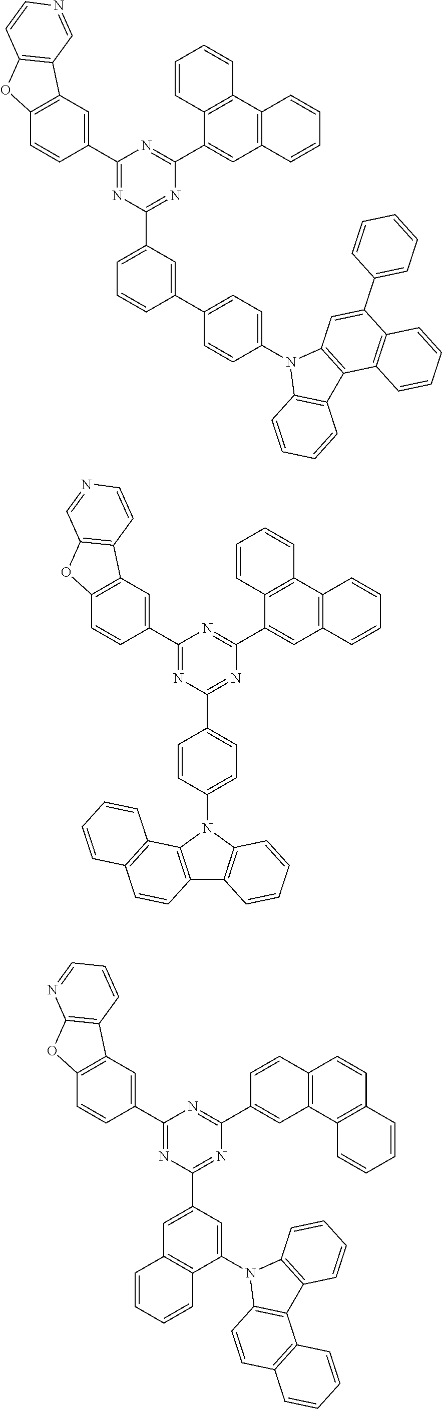 [SMILES: c1ccc(-c2cc3c(c4ccccc24)c2ccccc2n3-c2ccc(-c3cccc(-c4nc(-c5ccc6oc7ccncc7c6c5)nc(-c5cc6ccccc6c6ccccc56)n4)c3)cc2)cc1 ; c1ccc2c(c1)cc(-c1nc(-c3ccc(-n4c5ccccc5c5ccc6ccccc6c54)cc3)nc(-c3ccc4oc5cnccc5c4c3)n1)c1ccccc12 ; c1ccc2c(-n3c4ccccc4c4c5ccccc5ccc43)cc(-c3nc(-c4ccc5ccc6ccccc6c5c4)nc(-c4ccc5oc6ncccc6c5c4)n3)cc2c1]

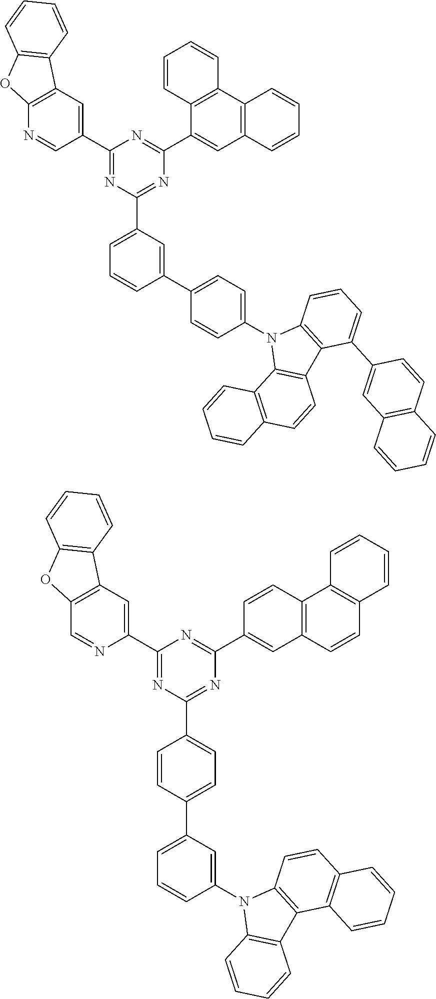 [SMILES: c1cc(-c2ccc(-n3c4cccc(-c5ccc6ccccc6c5)c4c4ccc5ccccc5c43)cc2)cc(-c2nc(-c3cnc4oc5ccccc5c4c3)nc(-c3cc4ccccc4c4ccccc34)n2)c1 ; c1cc(-c2ccc(-c3nc(-c4ccc5c(ccc6ccccc65)c4)nc(-c4cc5c(cn4)oc4ccccc45)n3)cc2)cc(-n2c3ccccc3c3c4ccccc4ccc32)c1]

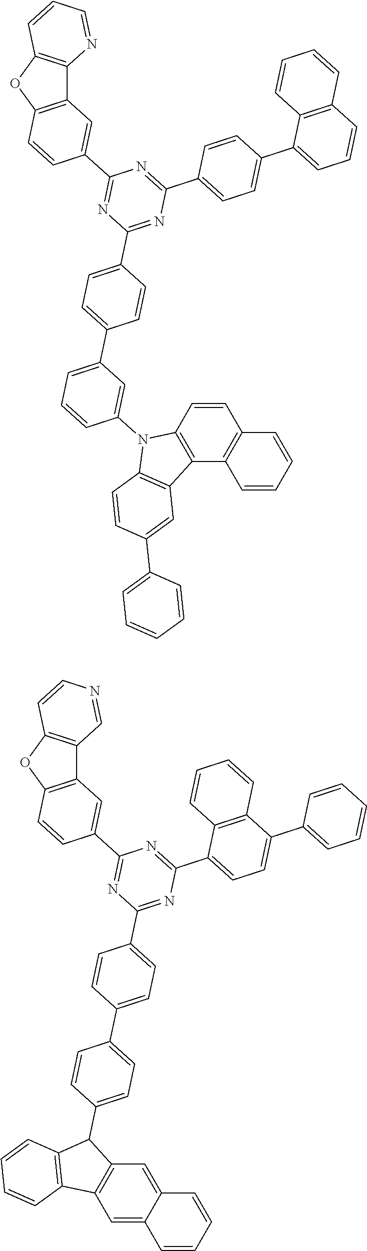 [SMILES: c1ccc(-c2ccc3c(c2)c2c4ccccc4ccc2n3-c2cccc(-c3ccc(-c4nc(-c5ccc(-c6cccc7ccccc67)cc5)nc(-c5ccc6oc7cccnc7c6c5)n4)cc3)c2)cc1 ; c1ccc(-c2ccc(-c3nc(-c4ccc(-c5ccc(C6c7ccccc7-c7cc8ccccc8cc76)cc5)cc4)nc(-c4ccc5oc6ccncc6c5c4)n3)c3ccccc23)cc1]

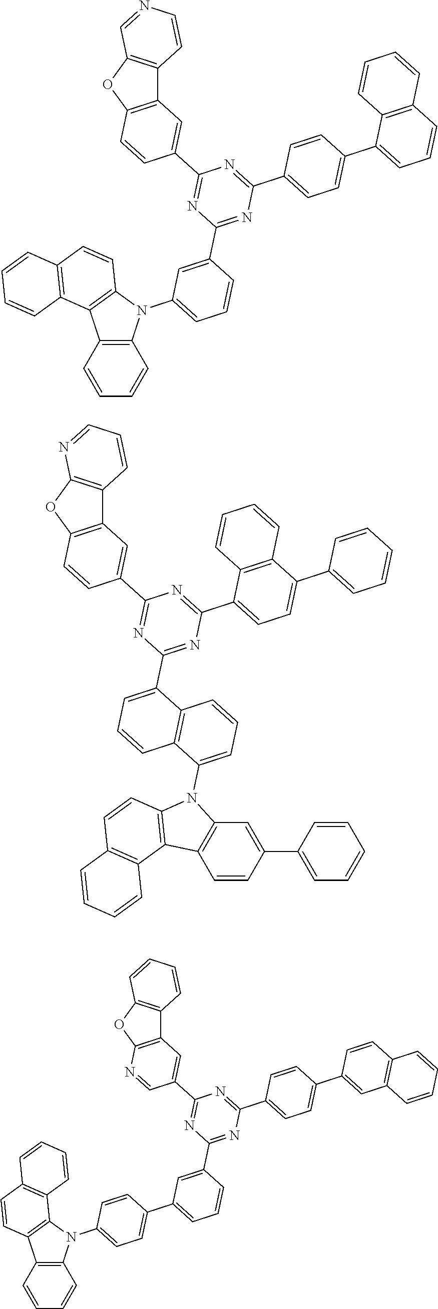

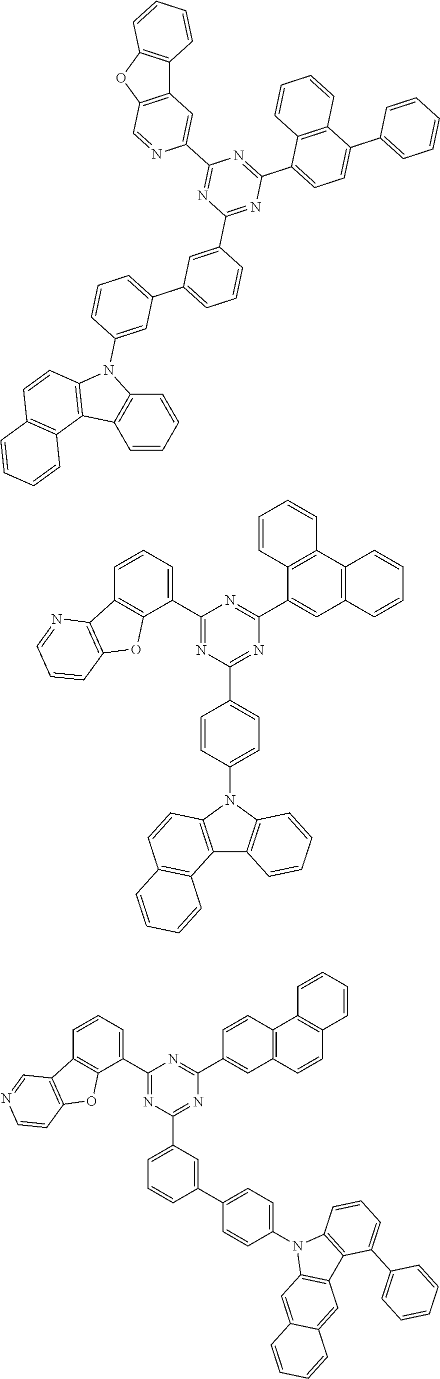 [SMILES: c1ccc(-c2ccc(-c3nc(-c4cccc(-c5cccc(-n6c7ccccc7c7c8ccccc8ccc76)c5)c4)nc(-c4cc5c(cn4)oc4ccccc45)n3)c3ccccc23)cc1 ; c1ccc2c(c1)cc(-c1nc(-c3ccc(-n4c5ccccc5c5c6ccccc6ccc54)cc3)nc(-c3cccc4c3oc3cccnc34)n1)c1ccccc12 ; c1ccc(-c2cccc3c2c2cc4ccccc4cc2n3-c2ccc(-c3cccc(-c4nc(-c5ccc6c(ccc7ccccc76)c5)nc(-c5cccc6c5oc5ccncc56)n4)c3)cc2)cc1]

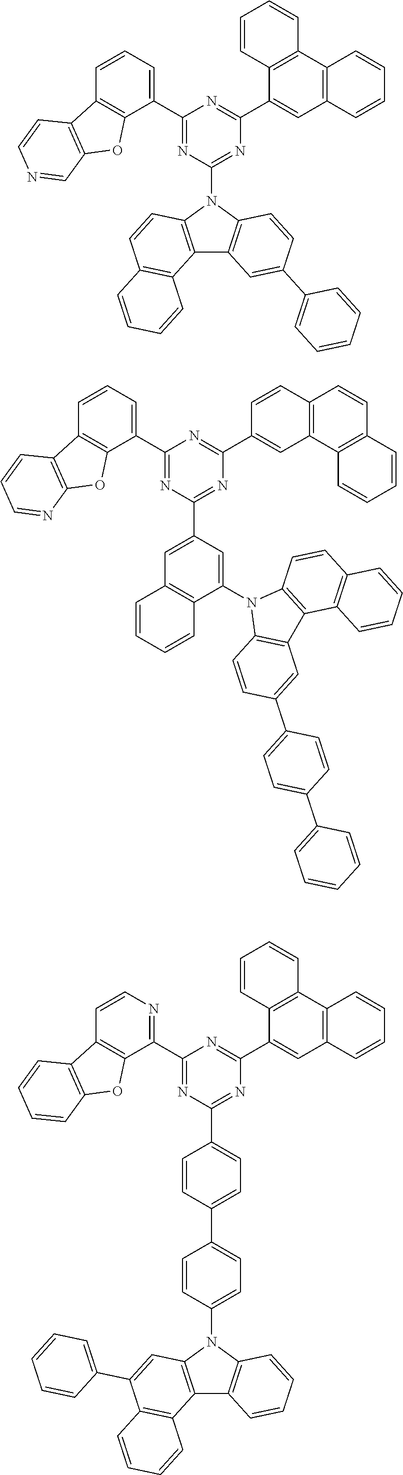

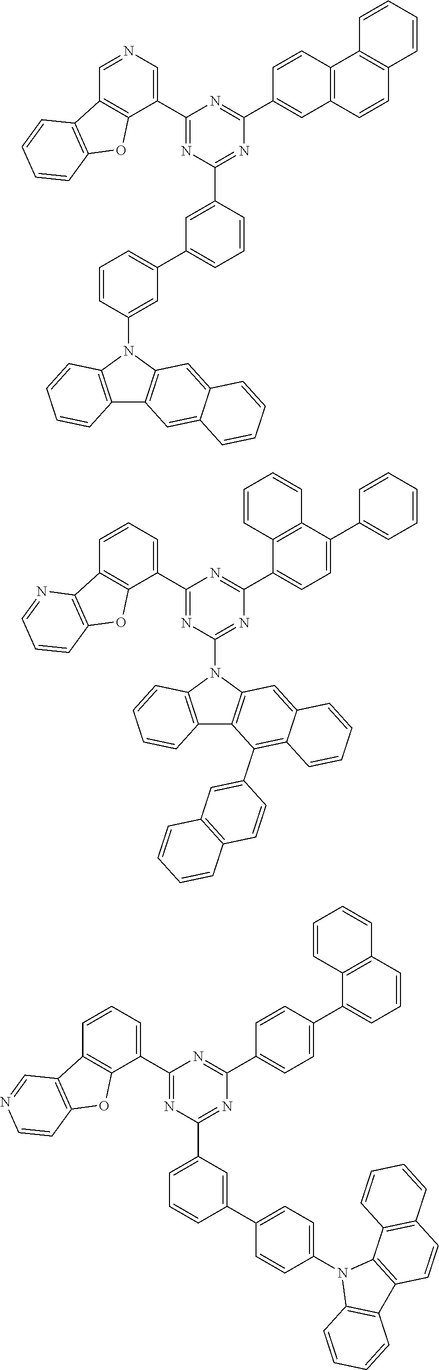

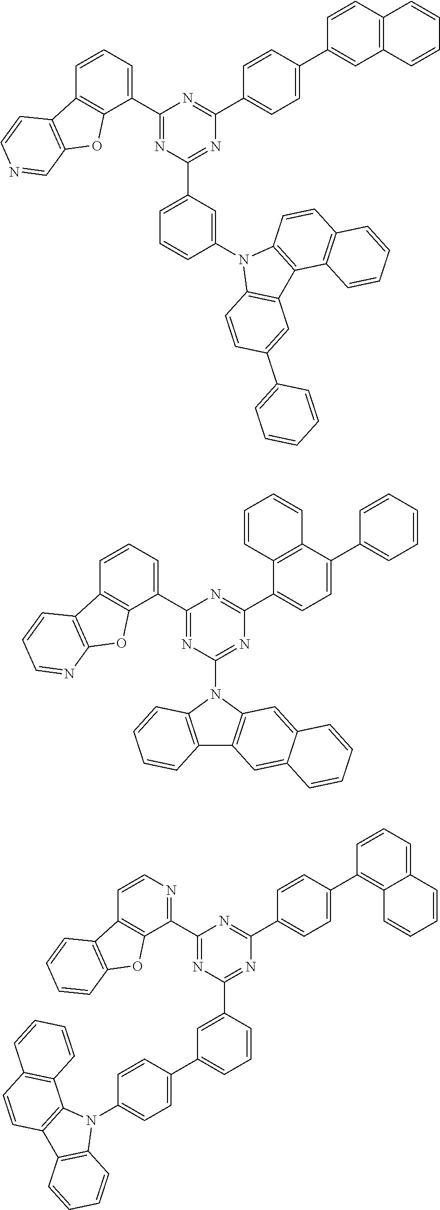 [SMILES: c1ccc(-c2ccc3c(c2)c2c4ccccc4ccc2n3-c2cccc(-c3nc(-c4ccc(-c5ccc6ccccc6c5)cc4)nc(-c4cccc5c4oc4cnccc45)n3)c2)cc1 ; c1ccc(-c2ccc(-c3nc(-c4cccc5c4oc4ncccc45)nc(-n4c5ccccc5c5cc6ccccc6cc54)n3)c3ccccc23)cc1 ; c1cc(-c2ccc(-n3c4ccccc4c4ccc5ccccc5c43)cc2)cc(-c2nc(-c3ccc(-c4cccc5ccccc45)cc3)nc(-c3nccc4c3oc3ccccc34)n2)c1]

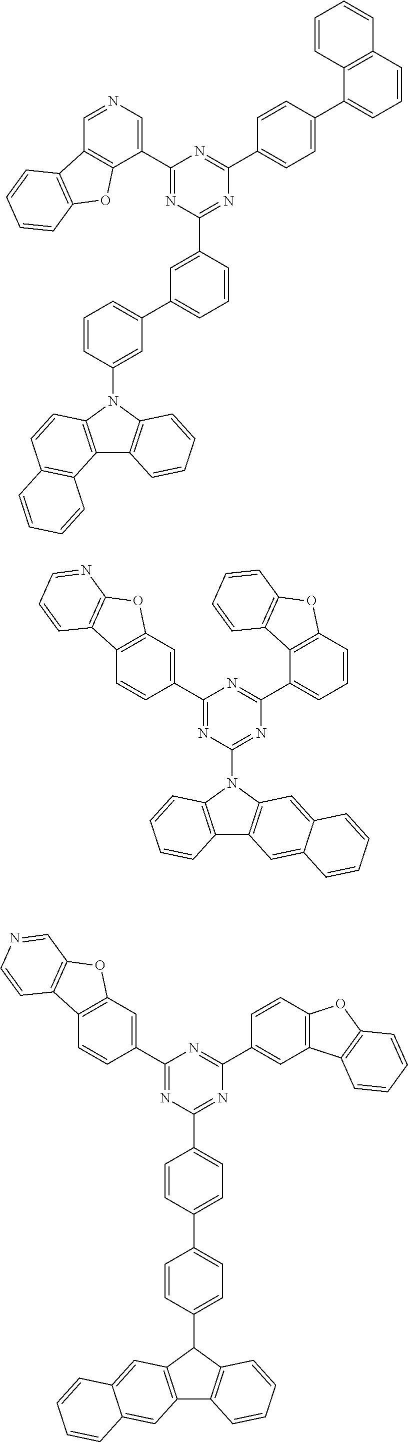

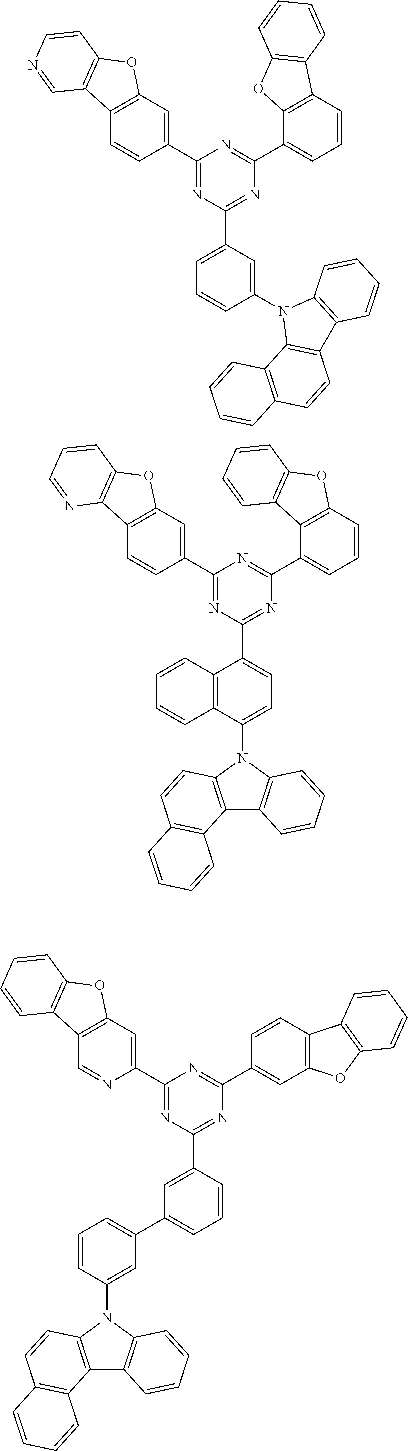

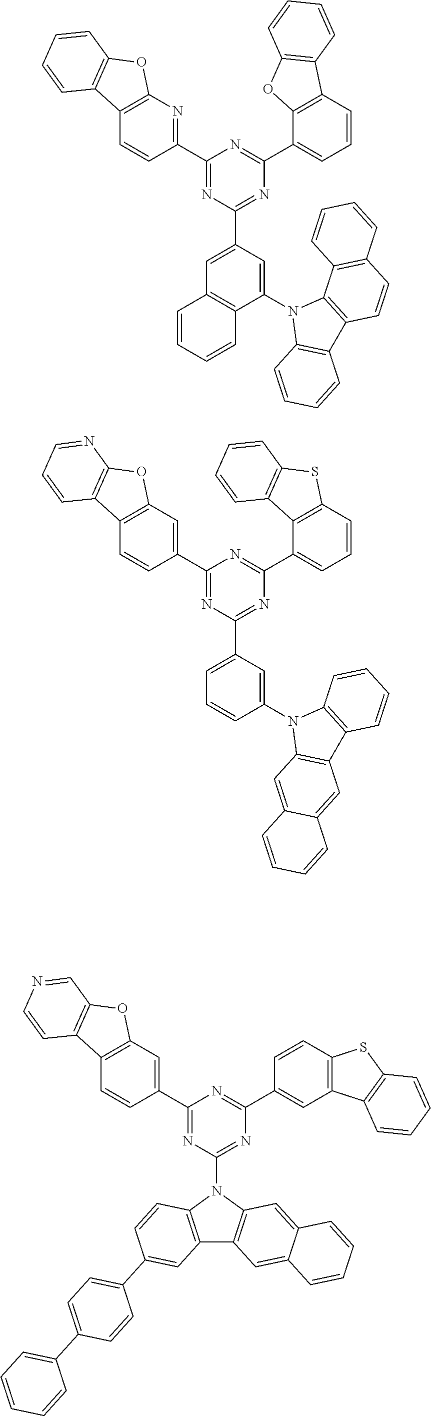

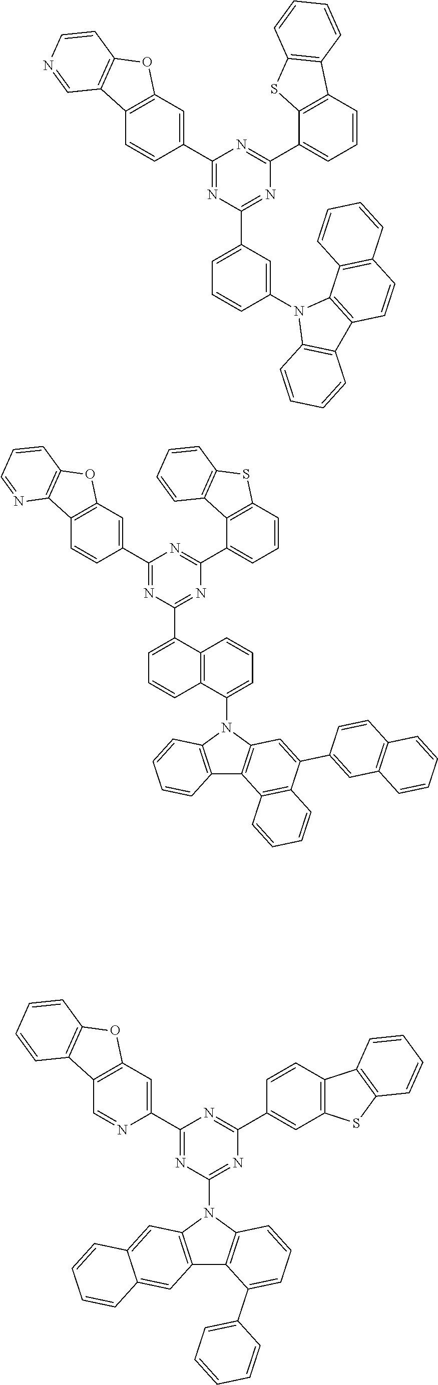 [SMILES: c1cc(-c2nc(-c3ccc4c(c3)oc3ccncc34)nc(-c3cccc4c3sc3ccccc34)n2)cc(-n2c3ccccc3c3ccc4ccccc4c32)c1 ; c1ccc2cc(-c3cc4c(c5ccccc35)c3ccccc3n4-c3cccc4c(-c5nc(-c6ccc7c(c6)oc6cccnc67)nc(-c6cccc7sc8ccccc8c67)n5)cccc34)ccc2c1 ; c1ccc(-c2cccc3c2c2cc4ccccc4cc2n3-c2nc(-c3ccc4c(c3)sc3ccccc34)nc(-c3cc4oc5ccccc5c4cn3)n2)cc1]

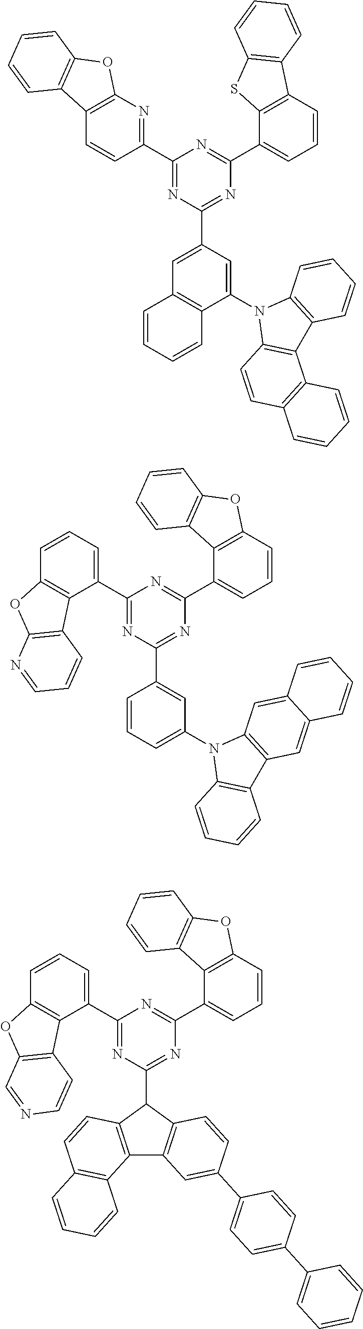 [SMILES: c1ccc2c(-n3c4ccccc4c4c5ccccc5ccc43)cc(-c3nc(-c4ccc5c(n4)oc4ccccc45)nc(-c4cccc5c4sc4ccccc45)n3)cc2c1 ; c1cc(-c2nc(-c3cccc4oc5ccccc5c34)nc(-c3cccc4oc5ncccc5c34)n2)cc(-n2c3ccccc3c3cc4ccccc4cc32)c1 ; c1ccc(-c2ccc(-c3ccc4c(c3)-c3c(ccc5ccccc35)C4c3nc(-c4cccc5oc6ccccc6c45)nc(-c4cccc5oc6cnccc6c45)n3)cc2)cc1]

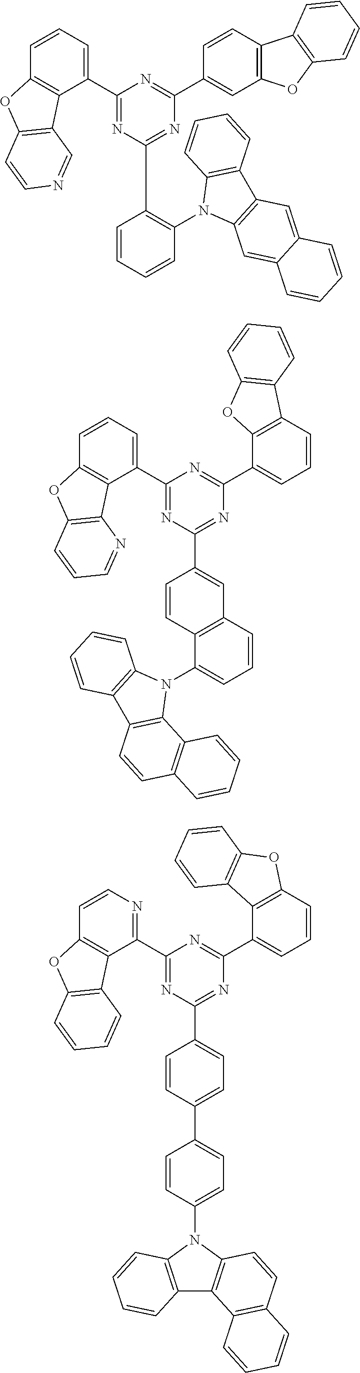

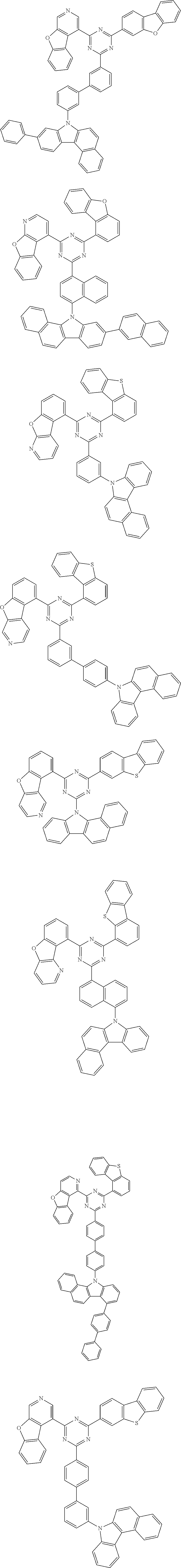

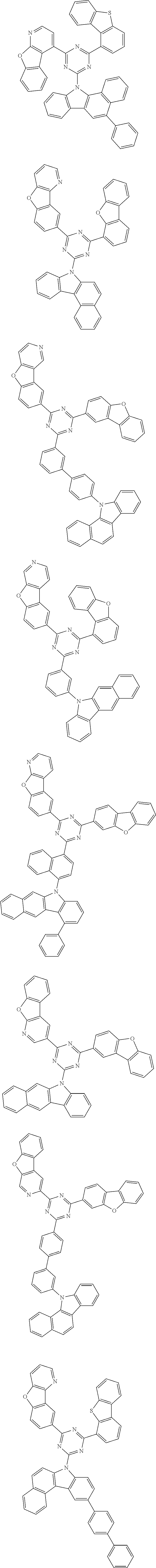

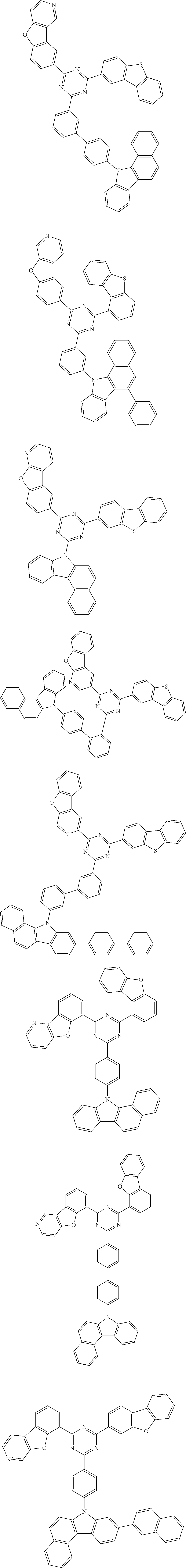

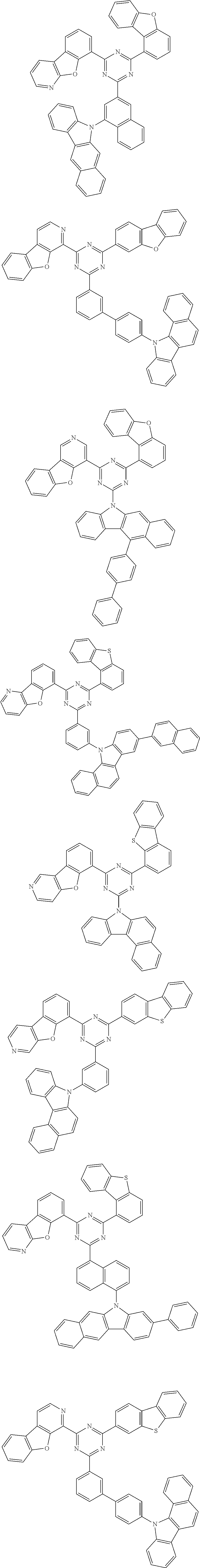

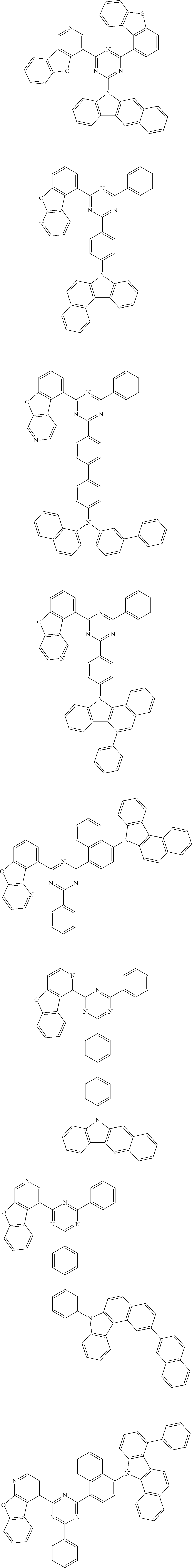

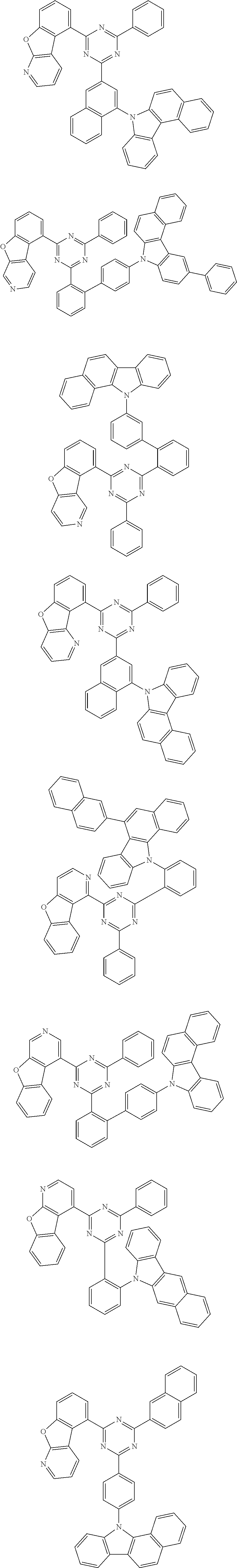

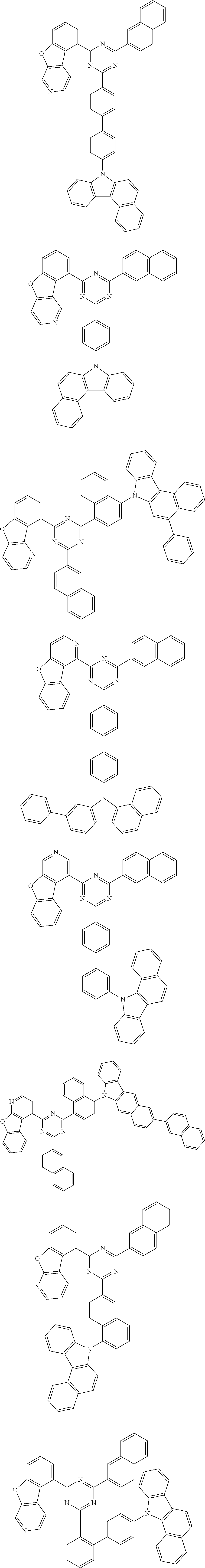

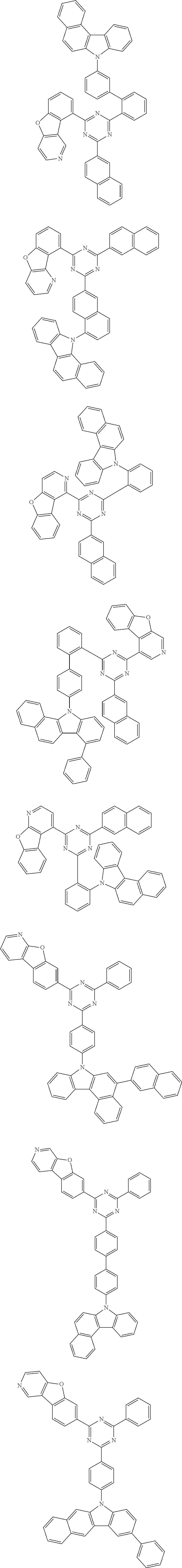

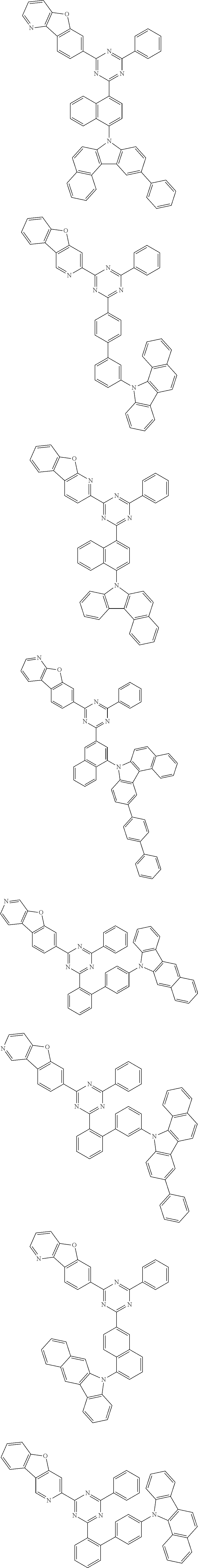

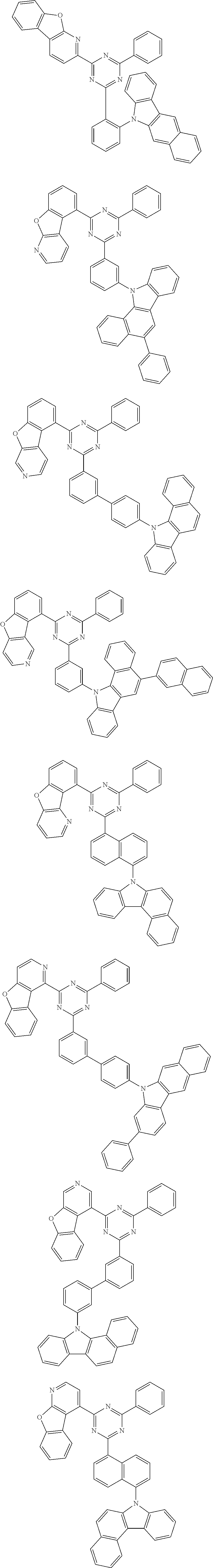

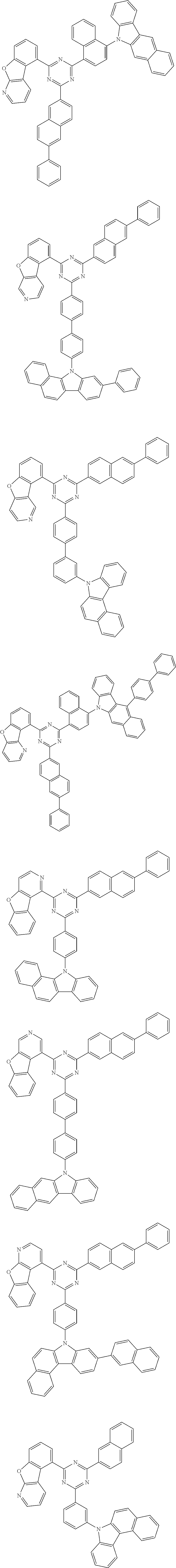

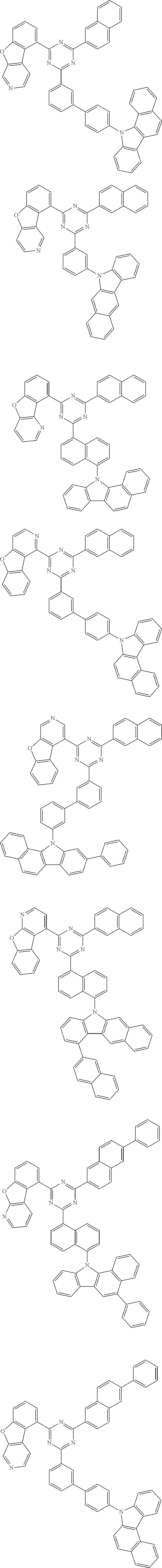

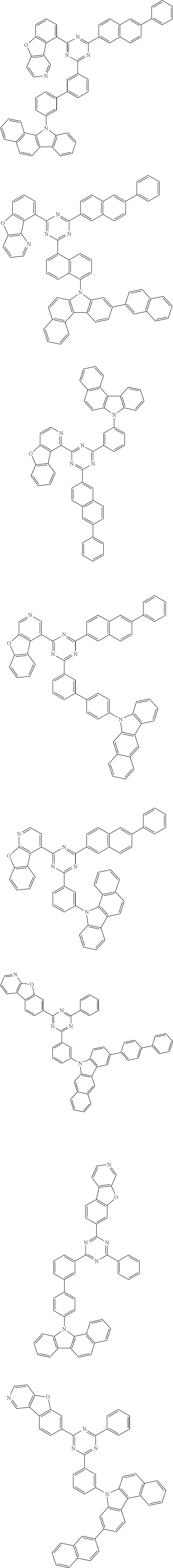

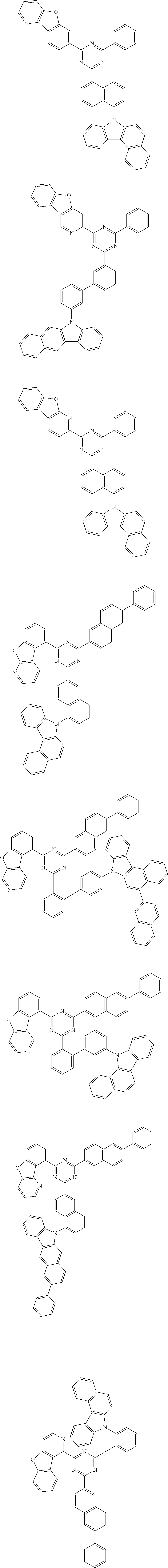

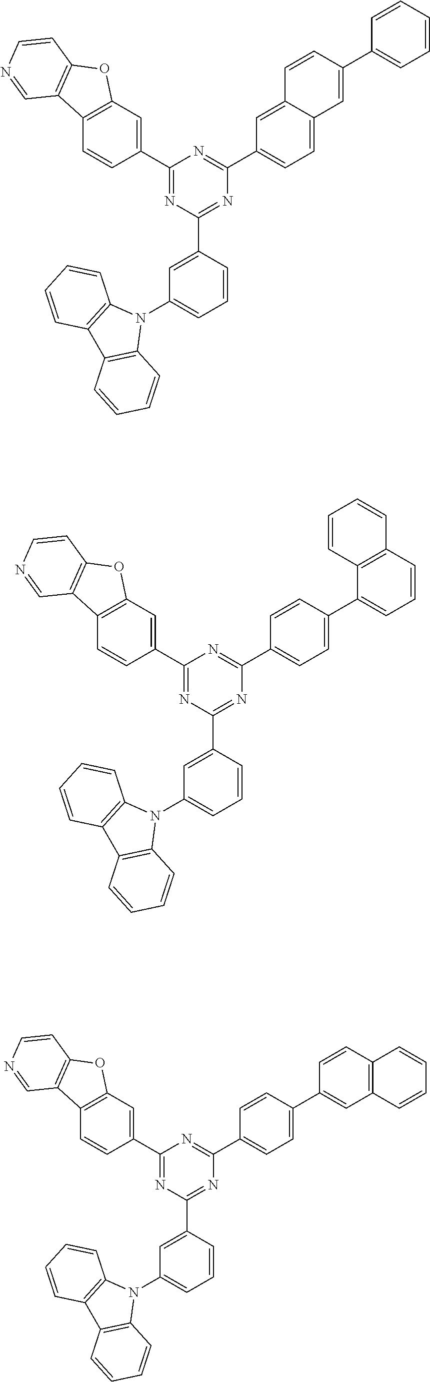 [SMILES: c1ccc(-c2ccc3cc(-c4nc(-c5cccc(-n6c7ccccc7c7ccccc76)c5)nc(-c5ccc6c(c5)oc5ccncc56)n4)ccc3c2)cc1 ; c1cc(-c2nc(-c3ccc(-c4cccc5ccccc45)cc3)nc(-c3ccc4c(c3)oc3ccncc34)n2)cc(-n2c3ccccc3c3ccccc32)c1 ; c1cc(-c2nc(-c3ccc(-c4ccc5ccccc5c4)cc3)nc(-c3ccc4c(c3)oc3ccncc34)n2)cc(-n2c3ccccc3c3ccccc32)c1]

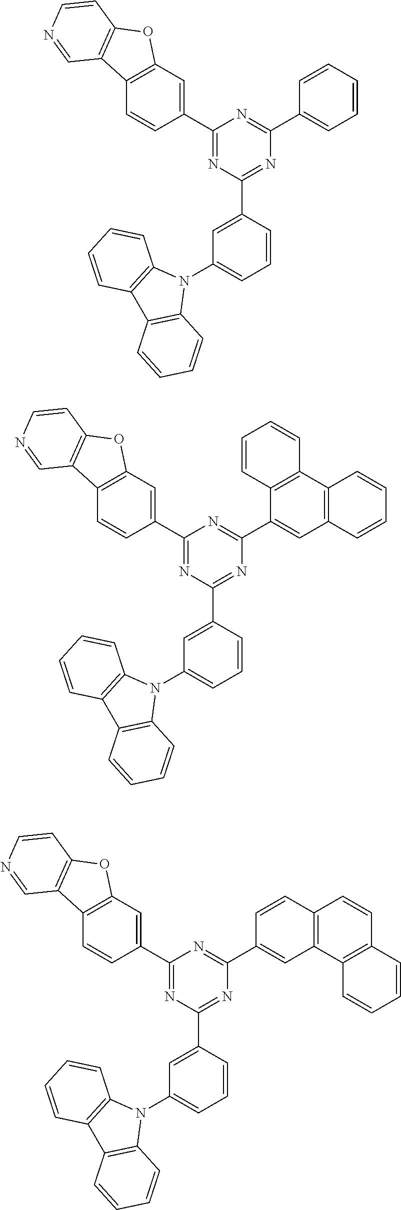 [SMILES: c1ccc(-c2nc(-c3cccc(-n4c5ccccc5c5ccccc54)c3)nc(-c3ccc4c(c3)oc3ccncc34)n2)cc1 ; c1cc(-c2nc(-c3ccc4c(c3)oc3ccncc34)nc(-c3cc4ccccc4c4ccccc34)n2)cc(-n2c3ccccc3c3ccccc32)c1 ; c1cc(-c2nc(-c3ccc4c(c3)oc3ccncc34)nc(-c3ccc4ccc5ccccc5c4c3)n2)cc(-n2c3ccccc3c3ccccc32)c1]

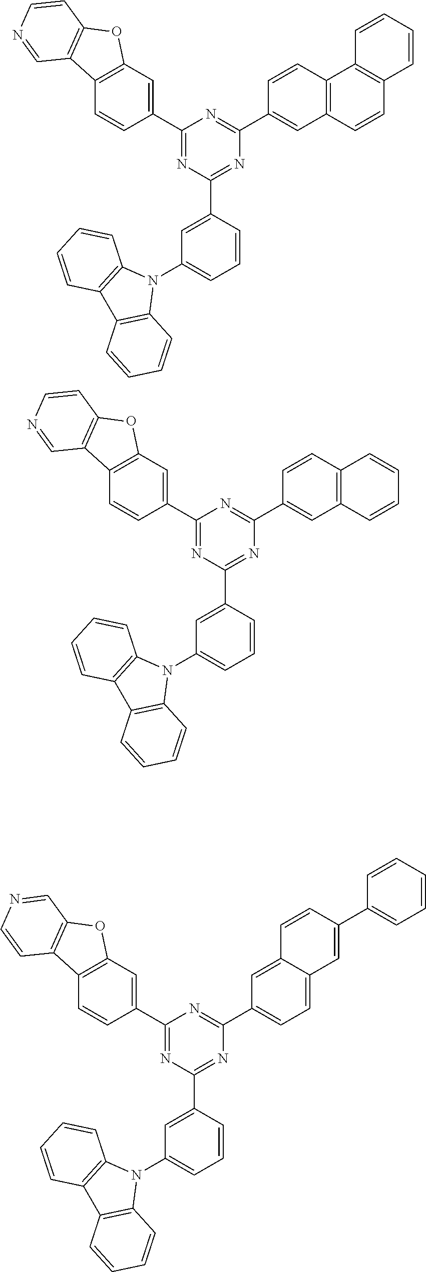

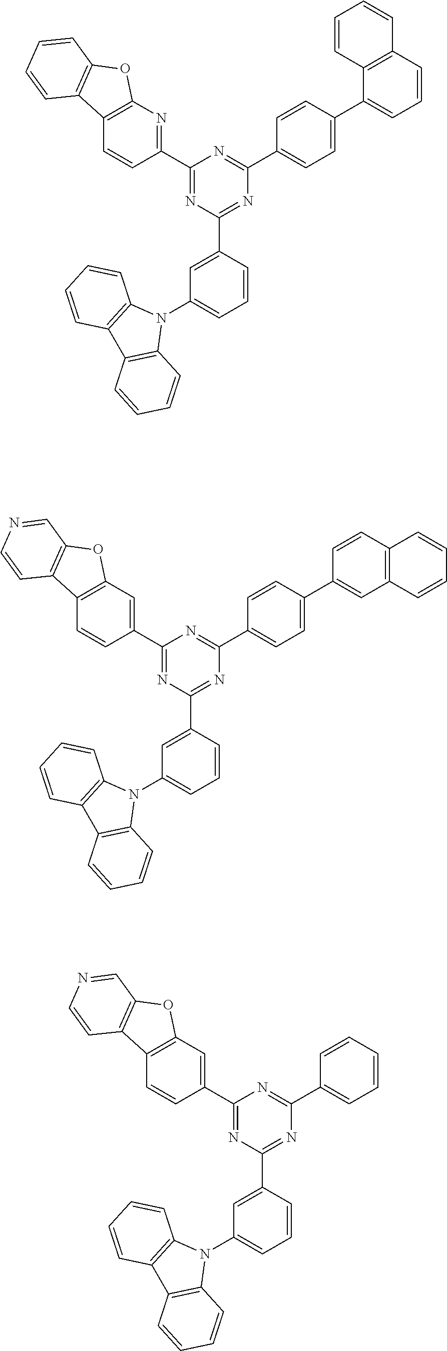 [SMILES: c1cc(-c2nc(-c3ccc(-c4cccc5ccccc45)cc3)nc(-c3ccc4c(n3)oc3ccccc34)n2)cc(-n2c3ccccc3c3ccccc32)c1 ; c1cc(-c2nc(-c3ccc(-c4ccc5ccccc5c4)cc3)nc(-c3ccc4c(c3)oc3cnccc34)n2)cc(-n2c3ccccc3c3ccccc32)c1 ; c1ccc(-c2nc(-c3cccc(-n4c5ccccc5c5ccccc54)c3)nc(-c3ccc4c(c3)oc3cnccc34)n2)cc1]

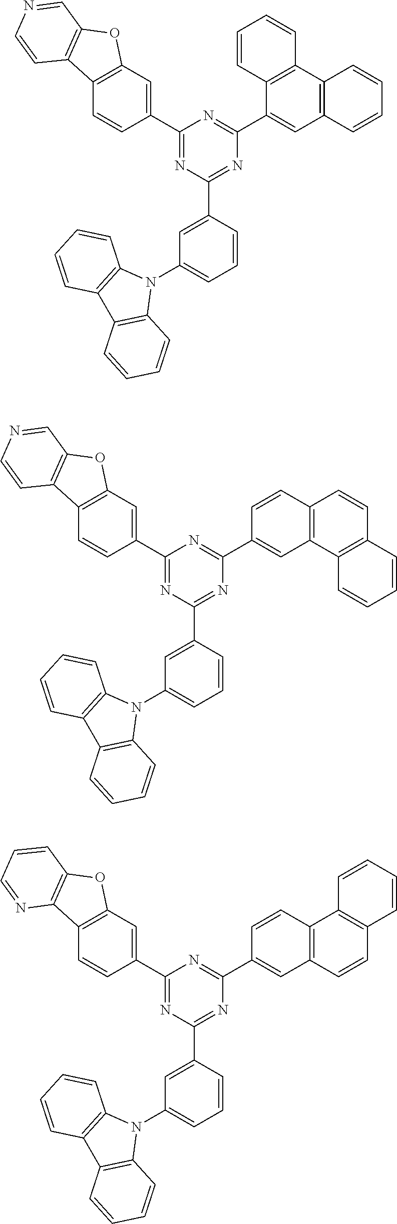 [SMILES: c1cc(-c2nc(-c3ccc4c(c3)oc3cnccc34)nc(-c3cc4ccccc4c4ccccc34)n2)cc(-n2c3ccccc3c3ccccc32)c1 ; c1cc(-c2nc(-c3ccc4c(c3)oc3cnccc34)nc(-c3ccc4ccc5ccccc5c4c3)n2)cc(-n2c3ccccc3c3ccccc32)c1 ; c1cc(-c2nc(-c3ccc4c(ccc5ccccc54)c3)nc(-c3ccc4c(c3)oc3cccnc34)n2)cc(-n2c3ccccc3c3ccccc32)c1]

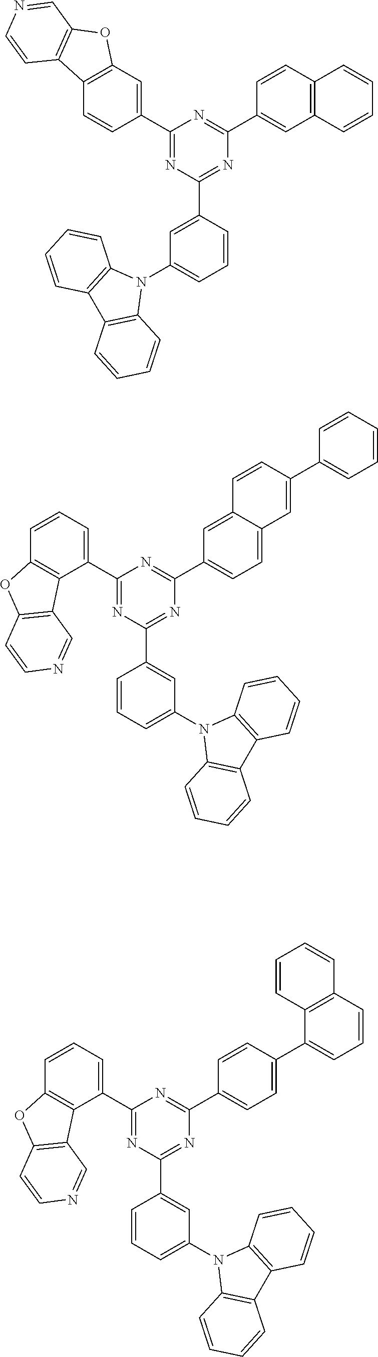 [SMILES: c1cc(-c2nc(-c3ccc4ccccc4c3)nc(-c3ccc4c(c3)oc3cnccc34)n2)cc(-n2c3ccccc3c3ccccc32)c1 ; c1ccc(-c2ccc3cc(-c4nc(-c5cccc(-n6c7ccccc7c7ccccc76)c5)nc(-c5cccc6oc7ccncc7c56)n4)ccc3c2)cc1 ; c1cc(-c2nc(-c3ccc(-c4cccc5ccccc45)cc3)nc(-c3cccc4oc5ccncc5c34)n2)cc(-n2c3ccccc3c3ccccc32)c1]

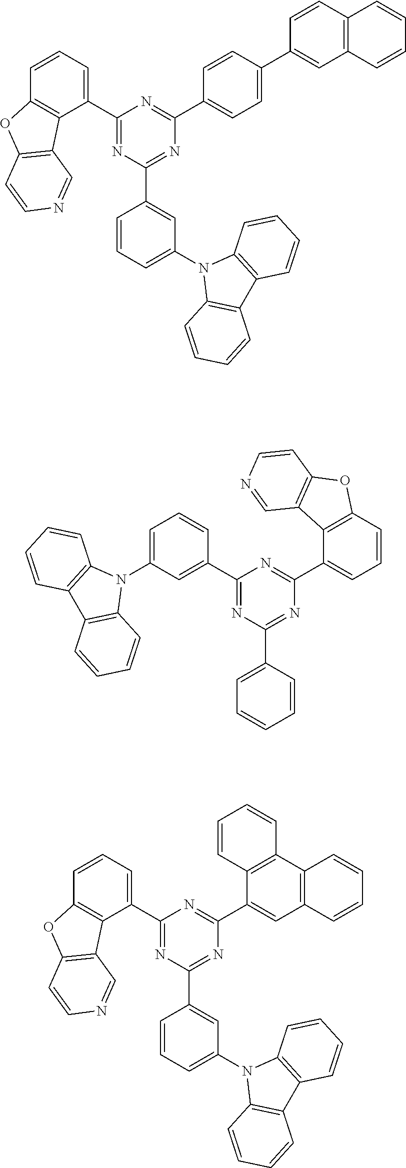 [SMILES: c1cc(-c2nc(-c3ccc(-c4ccc5ccccc5c4)cc3)nc(-c3cccc4oc5ccncc5c34)n2)cc(-n2c3ccccc3c3ccccc32)c1 ; c1ccc(-c2nc(-c3cccc(-n4c5ccccc5c5ccccc54)c3)nc(-c3cccc4oc5ccncc5c34)n2)cc1 ; c1cc(-c2nc(-c3cc4ccccc4c4ccccc34)nc(-c3cccc4oc5ccncc5c34)n2)cc(-n2c3ccccc3c3ccccc32)c1]

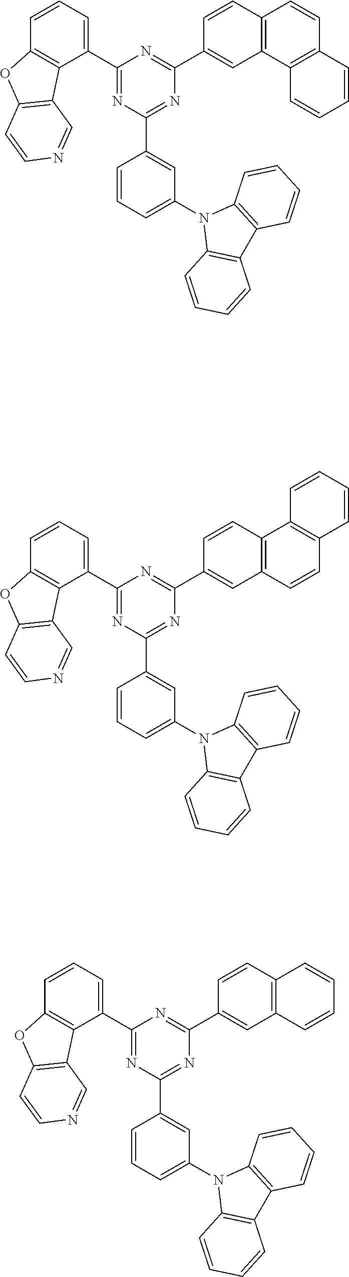 [SMILES: c1cc(-c2nc(-c3ccc4ccc5ccccc5c4c3)nc(-c3cccc4oc5ccncc5c34)n2)cc(-n2c3ccccc3c3ccccc32)c1 ; c1cc(-c2nc(-c3ccc4c(ccc5ccccc54)c3)nc(-c3cccc4oc5ccncc5c34)n2)cc(-n2c3ccccc3c3ccccc32)c1 ; c1cc(-c2nc(-c3ccc4ccccc4c3)nc(-c3cccc4oc5ccncc5c34)n2)cc(-n2c3ccccc3c3ccccc32)c1]

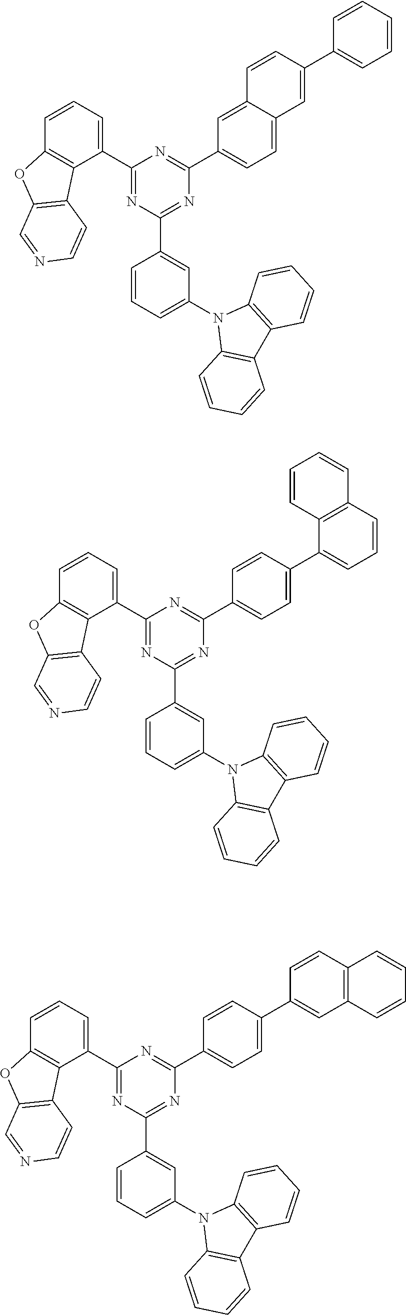 [SMILES: c1ccc(-c2ccc3cc(-c4nc(-c5cccc(-n6c7ccccc7c7ccccc76)c5)nc(-c5cccc6oc7cnccc7c56)n4)ccc3c2)cc1 ; c1cc(-c2nc(-c3ccc(-c4cccc5ccccc45)cc3)nc(-c3cccc4oc5cnccc5c34)n2)cc(-n2c3ccccc3c3ccccc32)c1 ; c1cc(-c2nc(-c3ccc(-c4ccc5ccccc5c4)cc3)nc(-c3cccc4oc5cnccc5c34)n2)cc(-n2c3ccccc3c3ccccc32)c1]

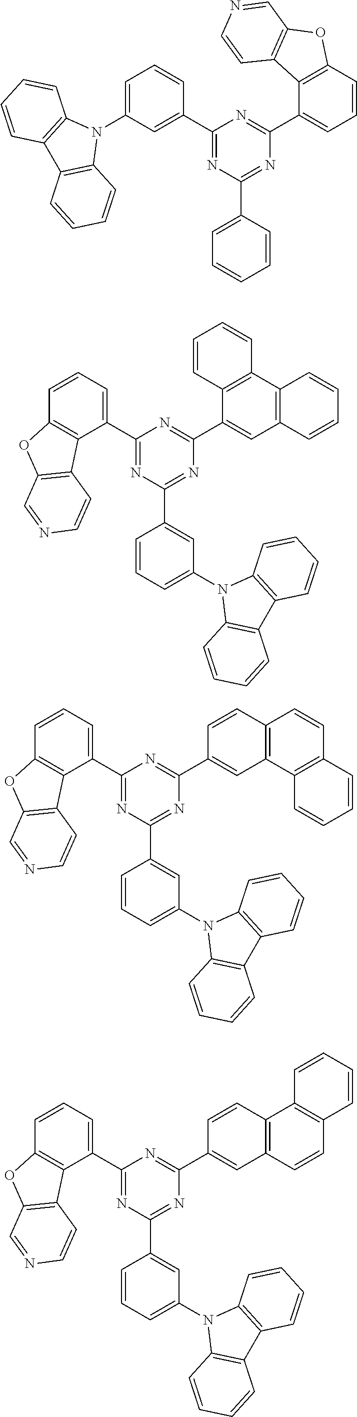

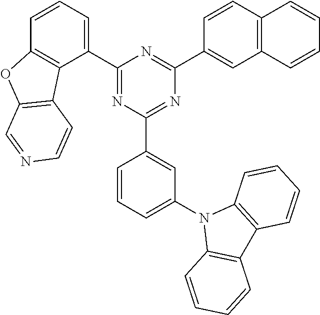 [SMILES: c1cc(-c2nc(-c3ccc4ccccc4c3)nc(-c3cccc4oc5cnccc5c34)n2)cc(-n2c3ccccc3c3ccccc32)c1]

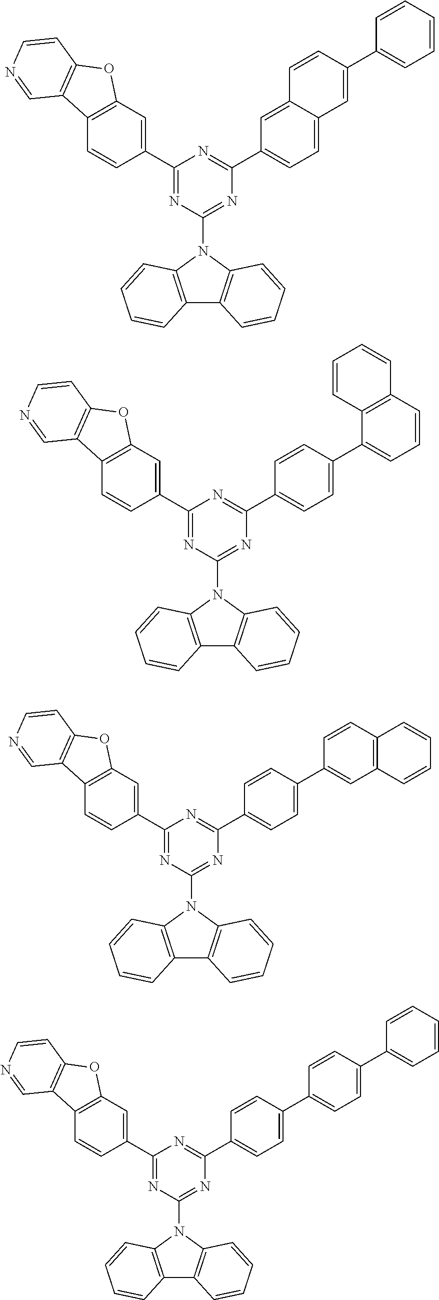 [SMILES: c1ccc(-c2ccc3cc(-c4nc(-c5ccc6c(c5)oc5ccncc56)nc(-n5c6ccccc6c6ccccc65)n4)ccc3c2)cc1 ; c1ccc2c(-c3ccc(-c4nc(-c5ccc6c(c5)oc5ccncc56)nc(-n5c6ccccc6c6ccccc65)n4)cc3)cccc2c1 ; c1ccc2cc(-c3ccc(-c4nc(-c5ccc6c(c5)oc5ccncc56)nc(-n5c6ccccc6c6ccccc65)n4)cc3)ccc2c1 ; c1ccc(-c2ccc(-c3ccc(-c4nc(-c5ccc6c(c5)oc5ccncc56)nc(-n5c6ccccc6c6ccccc65)n4)cc3)cc2)cc1]

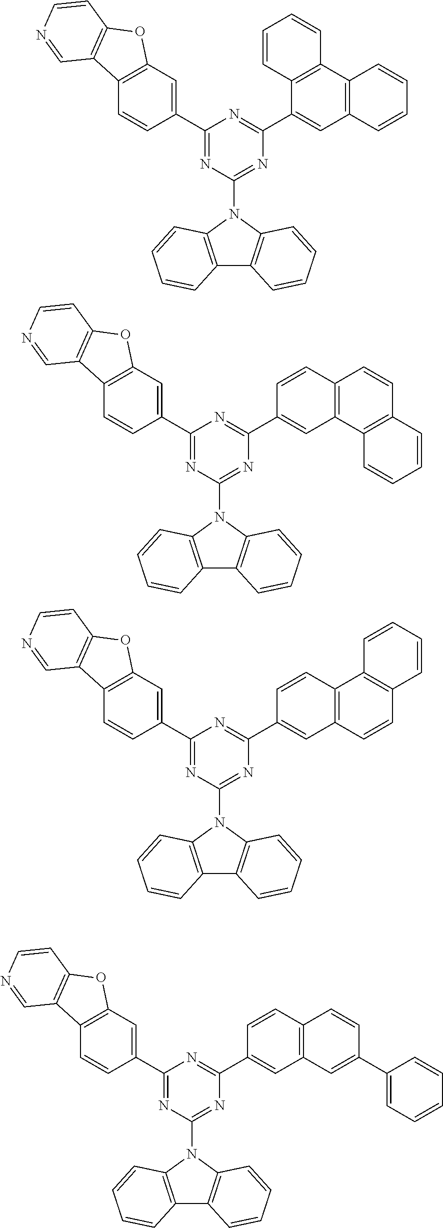 [SMILES: c1ccc2c(c1)cc(-c1nc(-c3ccc4c(c3)oc3ccncc34)nc(-n3c4ccccc4c4ccccc43)n1)c1ccccc12 ; c1ccc2c(c1)ccc1ccc(-c3nc(-c4ccc5c(c4)oc4ccncc45)nc(-n4c5ccccc5c5ccccc54)n3)cc12 ; c1ccc2c(c1)ccc1cc(-c3nc(-c4ccc5c(c4)oc4ccncc45)nc(-n4c5ccccc5c5ccccc54)n3)ccc12 ; c1ccc(-c2ccc3ccc(-c4nc(-c5ccc6c(c5)oc5ccncc56)nc(-n5c6ccccc6c6ccccc65)n4)cc3c2)cc1]

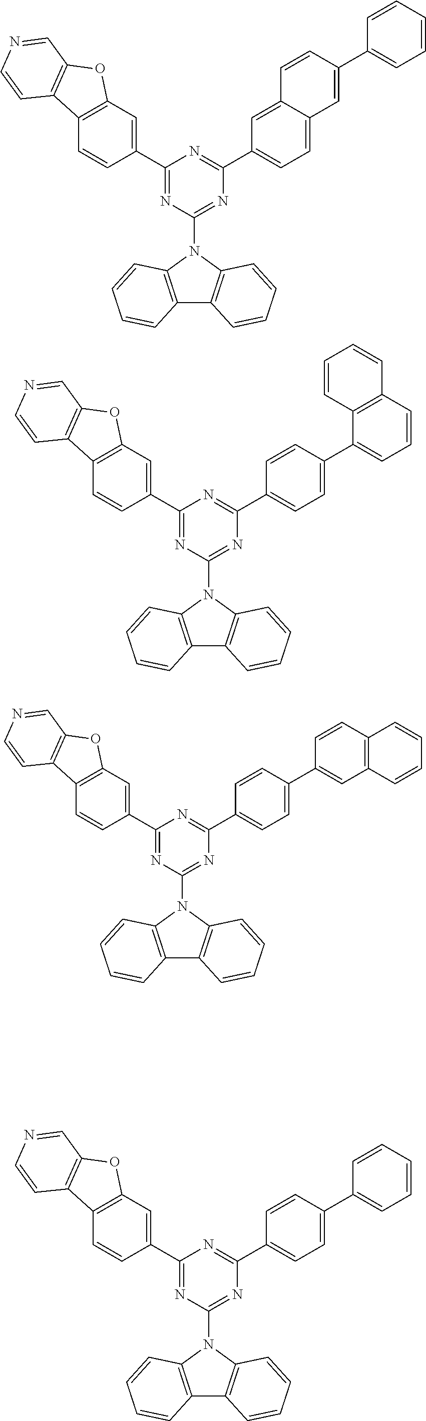 [SMILES: c1ccc(-c2ccc3cc(-c4nc(-c5ccc6c(c5)oc5cnccc56)nc(-n5c6ccccc6c6ccccc65)n4)ccc3c2)cc1 ; c1ccc2c(-c3ccc(-c4nc(-c5ccc6c(c5)oc5cnccc56)nc(-n5c6ccccc6c6ccccc65)n4)cc3)cccc2c1 ; c1ccc2cc(-c3ccc(-c4nc(-c5ccc6c(c5)oc5cnccc56)nc(-n5c6ccccc6c6ccccc65)n4)cc3)ccc2c1 ; c1ccc(-c2ccc(-c3nc(-c4ccc5c(c4)oc4cnccc45)nc(-n4c5ccccc5c5ccccc54)n3)cc2)cc1]

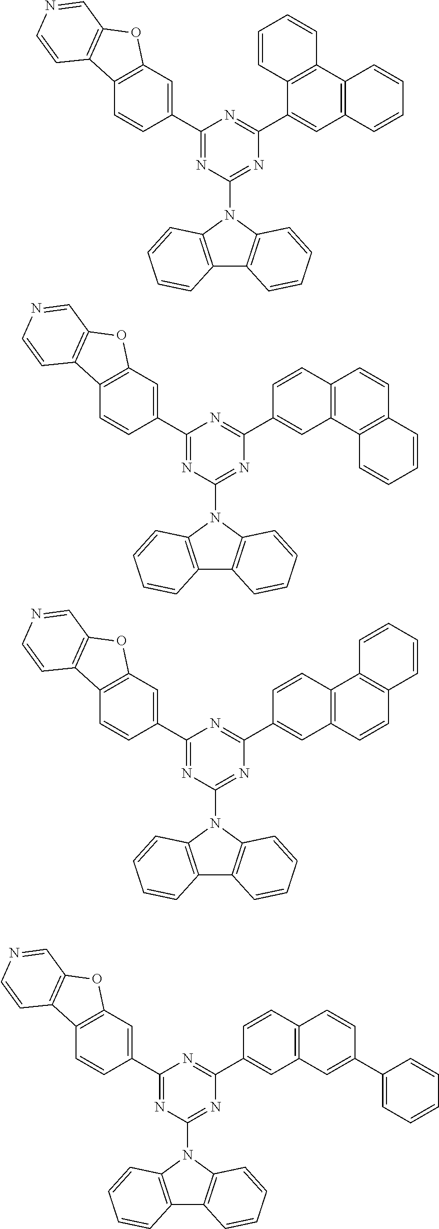 [SMILES: c1ccc2c(c1)cc(-c1nc(-c3ccc4c(c3)oc3cnccc34)nc(-n3c4ccccc4c4ccccc43)n1)c1ccccc12 ; c1ccc2c(c1)ccc1ccc(-c3nc(-c4ccc5c(c4)oc4cnccc45)nc(-n4c5ccccc5c5ccccc54)n3)cc12 ; c1ccc2c(c1)ccc1cc(-c3nc(-c4ccc5c(c4)oc4cnccc45)nc(-n4c5ccccc5c5ccccc54)n3)ccc12 ; c1ccc(-c2ccc3ccc(-c4nc(-c5ccc6c(c5)oc5cnccc56)nc(-n5c6ccccc6c6ccccc65)n4)cc3c2)cc1]

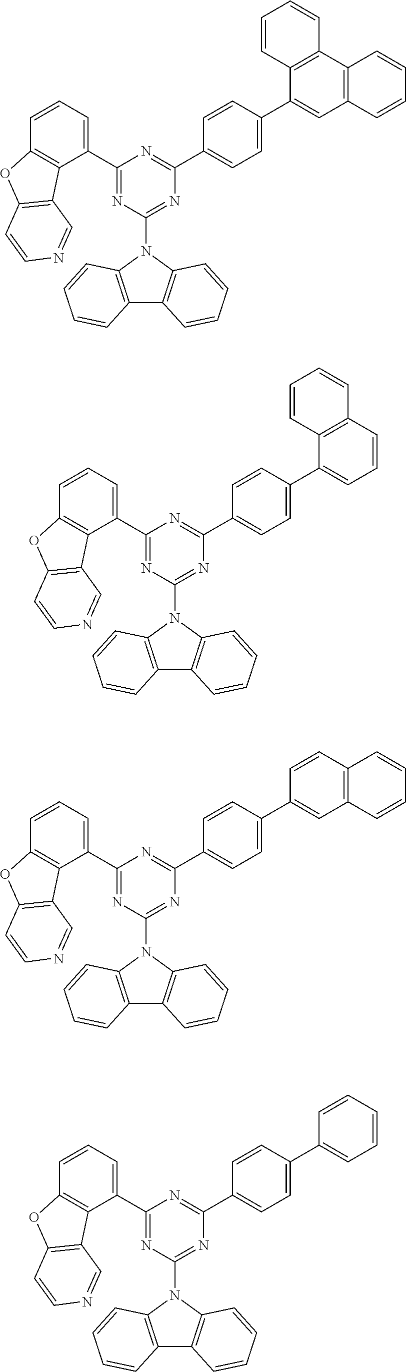 [SMILES: c1ccc2c(c1)cc(-c1ccc(-c3nc(-c4cccc5oc6ccncc6c45)nc(-n4c5ccccc5c5ccccc54)n3)cc1)c1ccccc12 ; c1ccc2c(-c3ccc(-c4nc(-c5cccc6oc7ccncc7c56)nc(-n5c6ccccc6c6ccccc65)n4)cc3)cccc2c1 ; c1ccc2cc(-c3ccc(-c4nc(-c5cccc6oc7ccncc7c56)nc(-n5c6ccccc6c6ccccc65)n4)cc3)ccc2c1 ; c1ccc(-c2ccc(-c3nc(-c4cccc5oc6ccncc6c45)nc(-n4c5ccccc5c5ccccc54)n3)cc2)cc1]

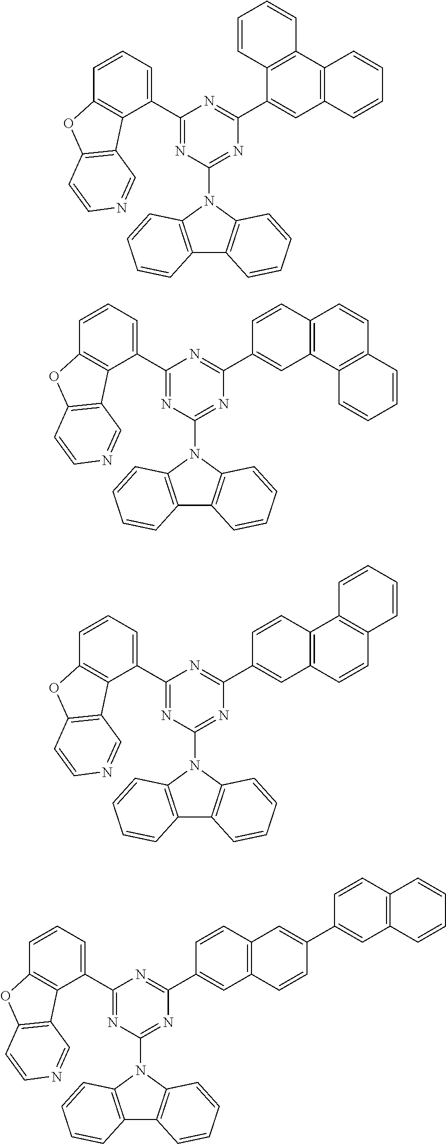

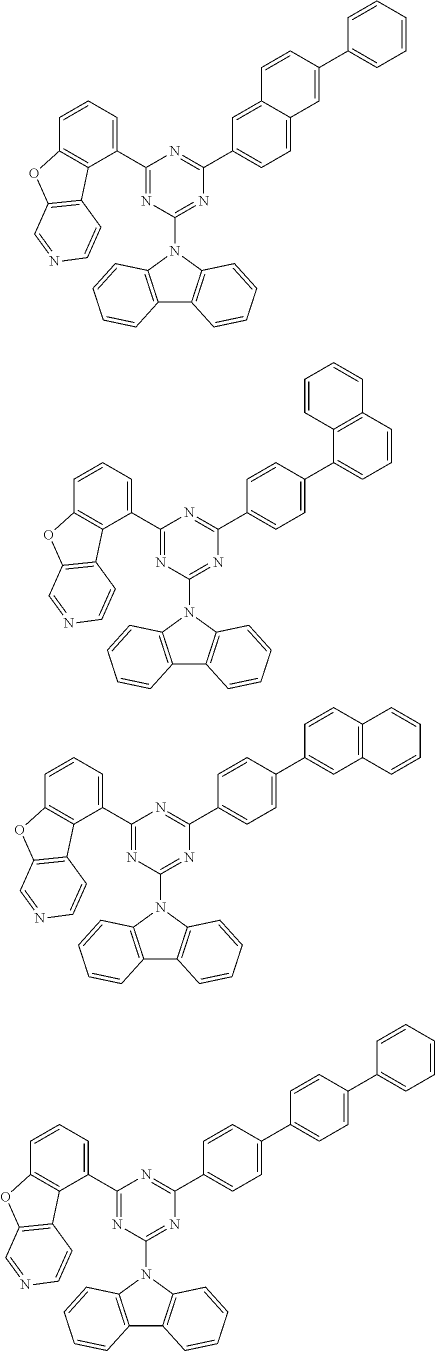 [SMILES: c1ccc(-c2ccc3cc(-c4nc(-c5cccc6oc7cnccc7c56)nc(-n5c6ccccc6c6ccccc65)n4)ccc3c2)cc1 ; c1ccc2c(-c3ccc(-c4nc(-c5cccc6oc7cnccc7c56)nc(-n5c6ccccc6c6ccccc65)n4)cc3)cccc2c1 ; c1ccc2cc(-c3ccc(-c4nc(-c5cccc6oc7cnccc7c56)nc(-n5c6ccccc6c6ccccc65)n4)cc3)ccc2c1 ; c1ccc(-c2ccc(-c3ccc(-c4nc(-c5cccc6oc7cnccc7c56)nc(-n5c6ccccc6c6ccccc65)n4)cc3)cc2)cc1]

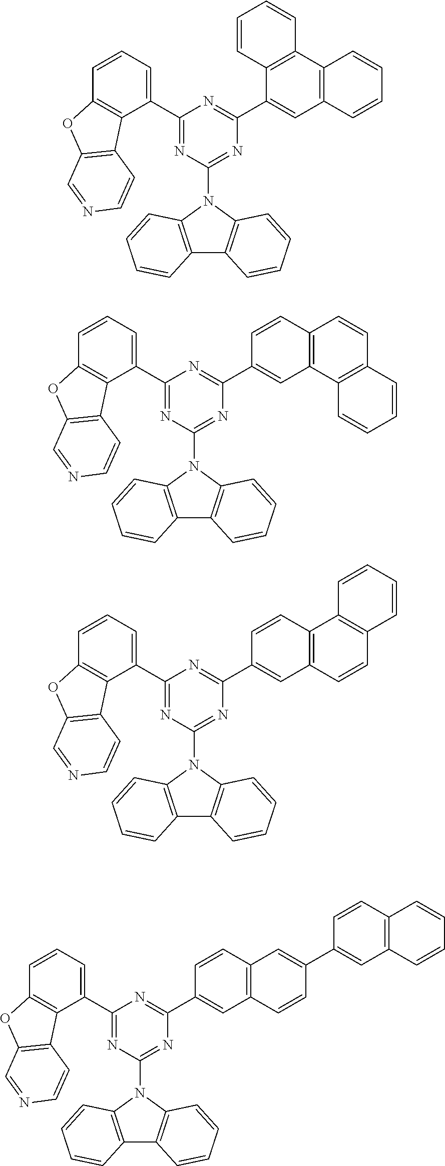

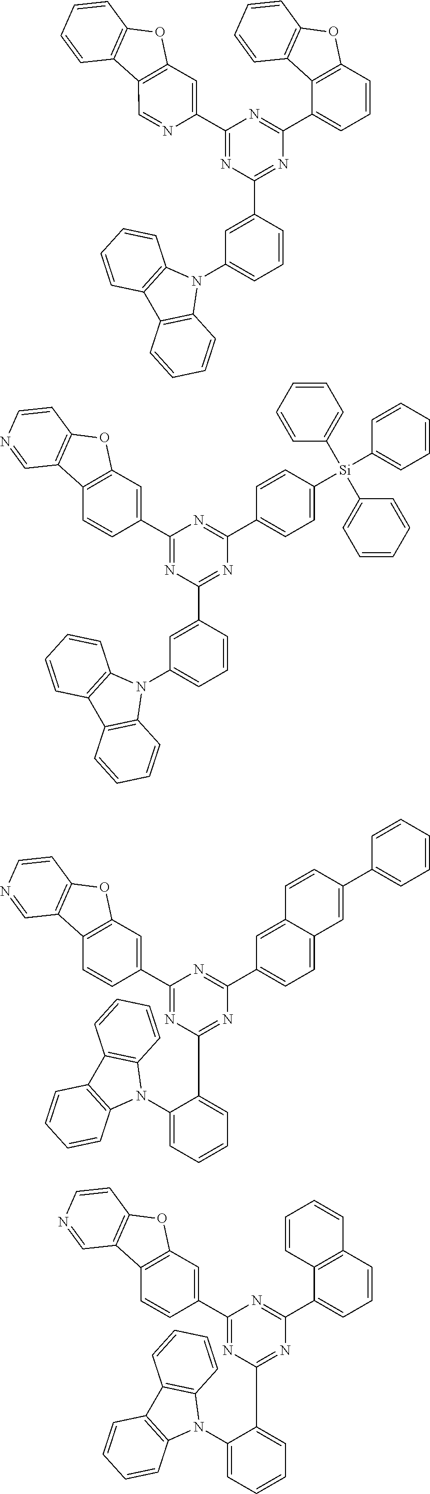

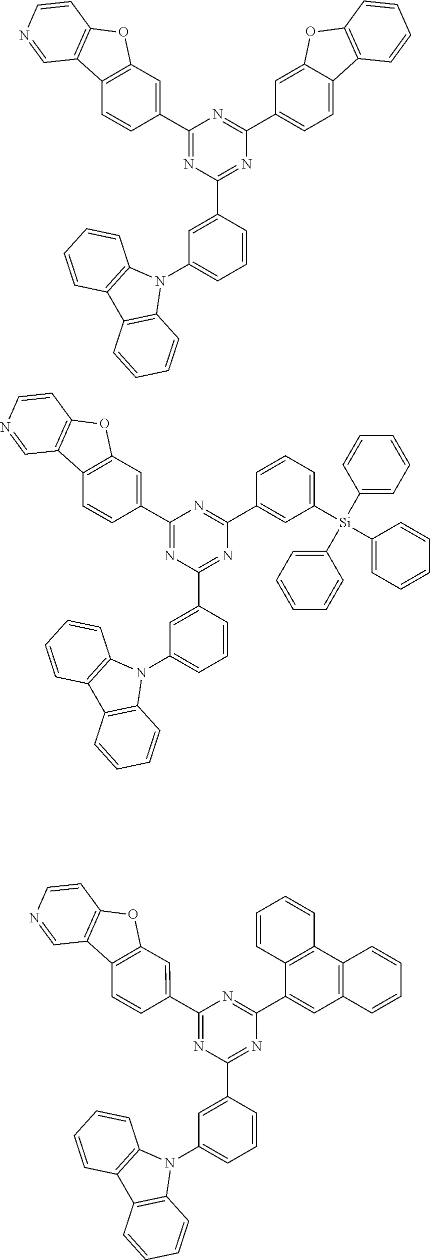 [SMILES: c1cc(-c2nc(-c3ccc4c(c3)oc3ccccc34)nc(-c3ccc4c(c3)oc3ccncc34)n2)cc(-n2c3ccccc3c3ccccc32)c1 ; c1ccc([Si](c2ccccc2)(c2ccccc2)c2cccc(-c3nc(-c4cccc(-n5c6ccccc6c6ccccc65)c4)nc(-c4ccc5c(c4)oc4ccncc45)n3)c2)cc1 ; c1cc(-c2nc(-c3ccc4c(c3)oc3ccncc34)nc(-c3cc4ccccc4c4ccccc34)n2)cc(-n2c3ccccc3c3ccccc32)c1]

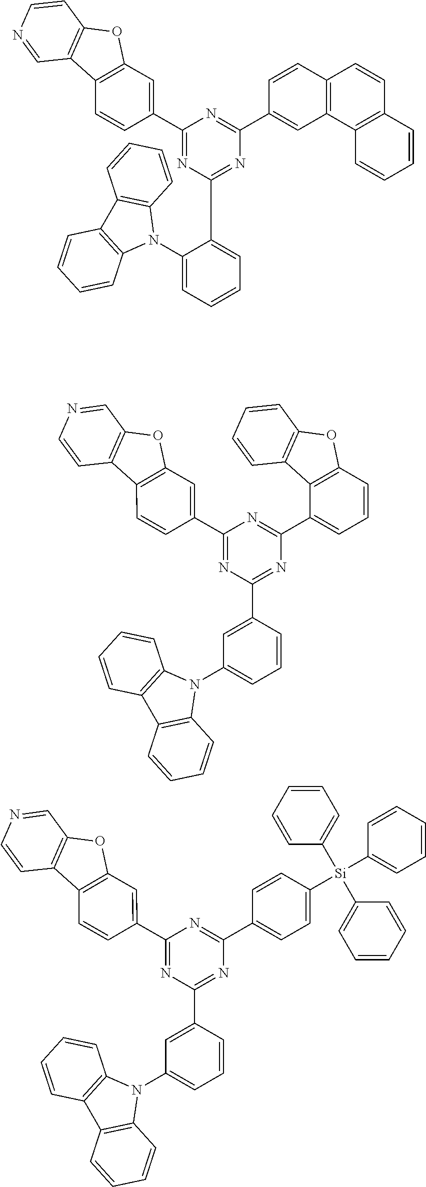 [SMILES: c1ccc(-n2c3ccccc3c3ccccc32)c(-c2nc(-c3ccc4c(c3)oc3ccncc34)nc(-c3ccc4ccc5ccccc5c4c3)n2)c1 ; c1cc(-c2nc(-c3ccc4c(c3)oc3cnccc34)nc(-c3cccc4oc5ccccc5c34)n2)cc(-n2c3ccccc3c3ccccc32)c1 ; c1ccc([Si](c2ccccc2)(c2ccccc2)c2ccc(-c3nc(-c4cccc(-n5c6ccccc6c6ccccc65)c4)nc(-c4ccc5c(c4)oc4cnccc45)n3)cc2)cc1]

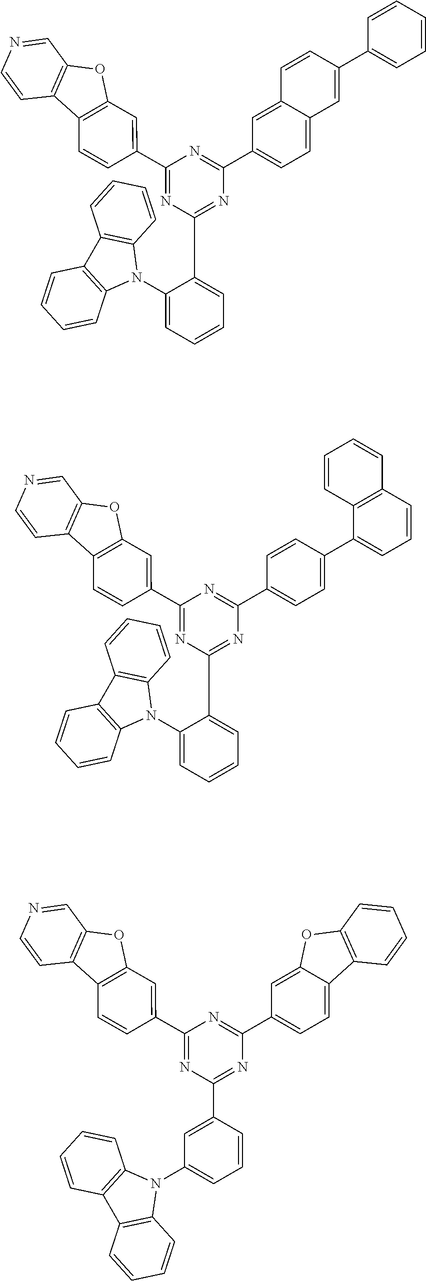 [SMILES: c1ccc(-c2ccc3cc(-c4nc(-c5ccc6c(c5)oc5cnccc56)nc(-c5ccccc5-n5c6ccccc6c6ccccc65)n4)ccc3c2)cc1 ; c1ccc(-n2c3ccccc3c3ccccc32)c(-c2nc(-c3ccc(-c4cccc5ccccc45)cc3)nc(-c3ccc4c(c3)oc3cnccc34)n2)c1 ; c1cc(-c2nc(-c3ccc4c(c3)oc3ccccc34)nc(-c3ccc4c(c3)oc3cnccc34)n2)cc(-n2c3ccccc3c3ccccc32)c1]

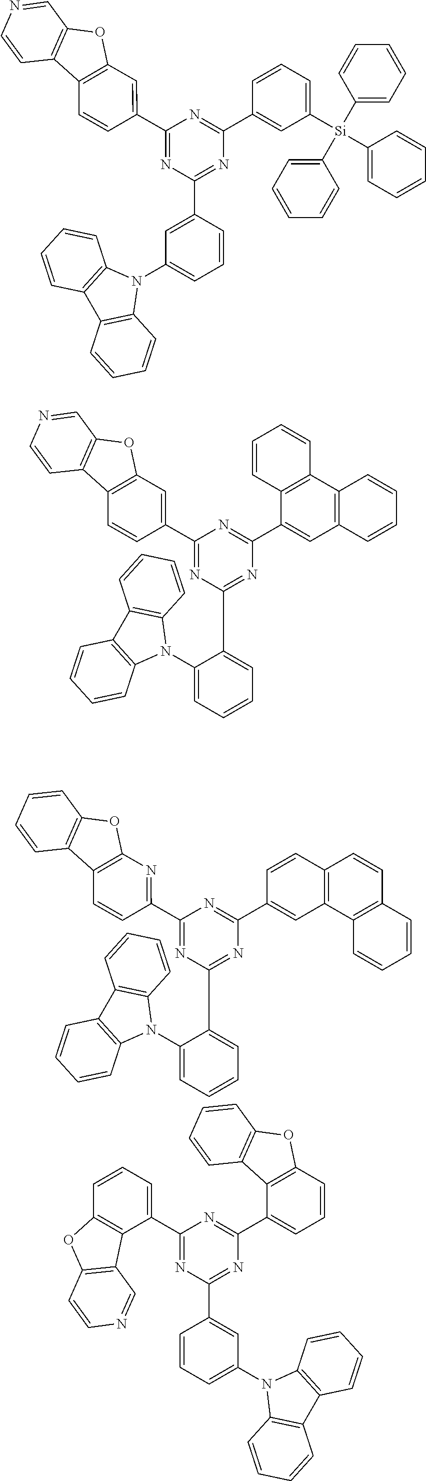

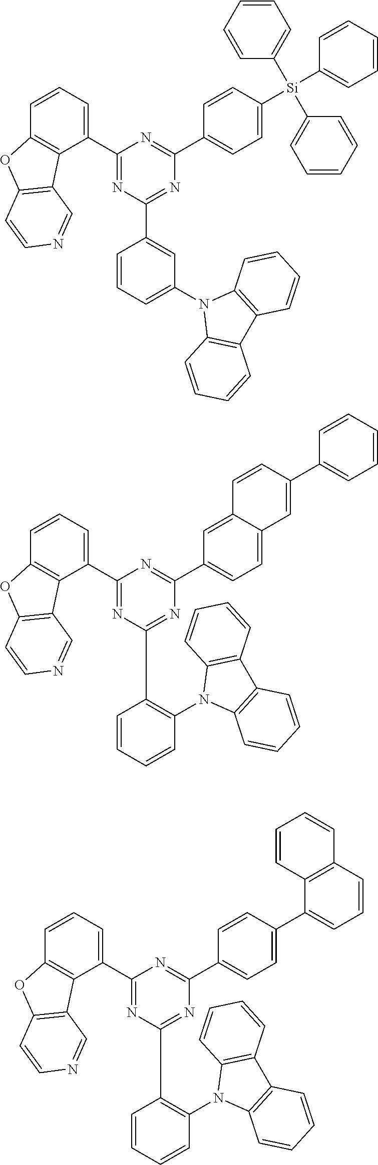 [SMILES: c1ccc([Si](c2ccccc2)(c2ccccc2)c2ccc(-c3nc(-c4cccc(-n5c6ccccc6c6ccccc65)c4)nc(-c4cccc5oc6ccncc6c45)n3)cc2)cc1 ; c1ccc(-c2ccc3cc(-c4nc(-c5ccccc5-n5c6ccccc6c6ccccc65)nc(-c5cccc6oc7ccncc7c56)n4)ccc3c2)cc1 ; c1ccc(-n2c3ccccc3c3ccccc32)c(-c2nc(-c3ccc(-c4cccc5ccccc45)cc3)nc(-c3cccc4oc5ccncc5c34)n2)c1]

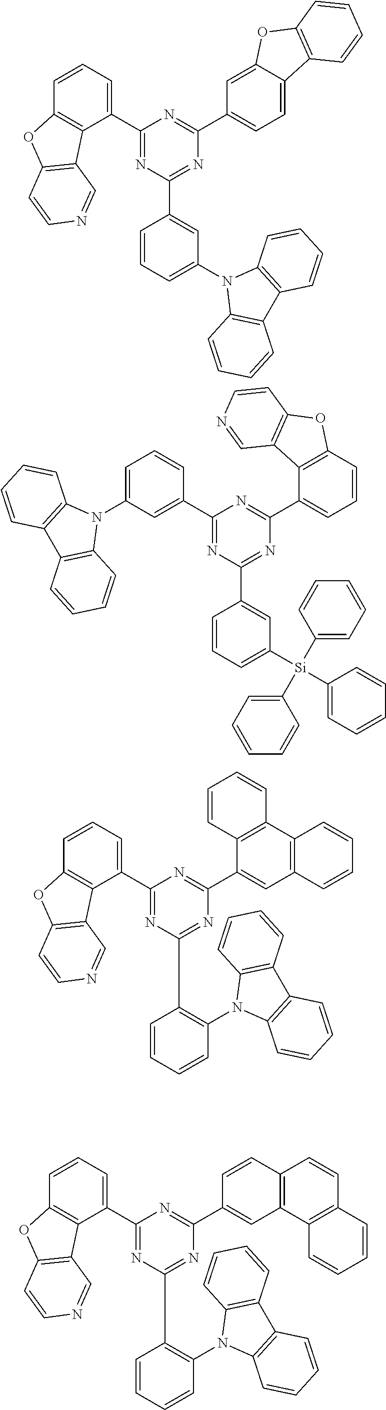

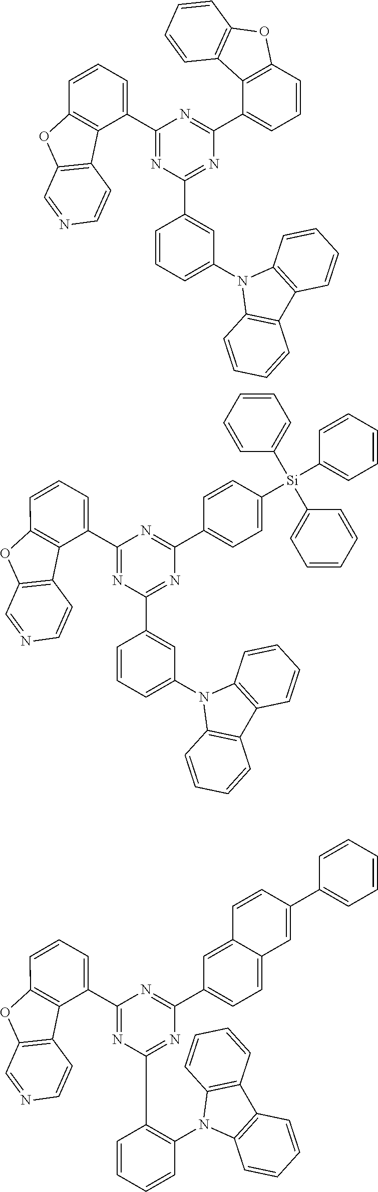

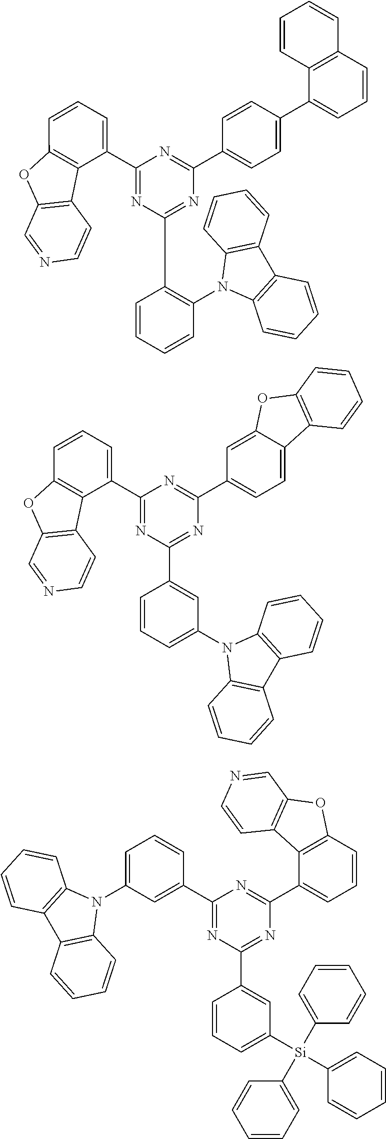

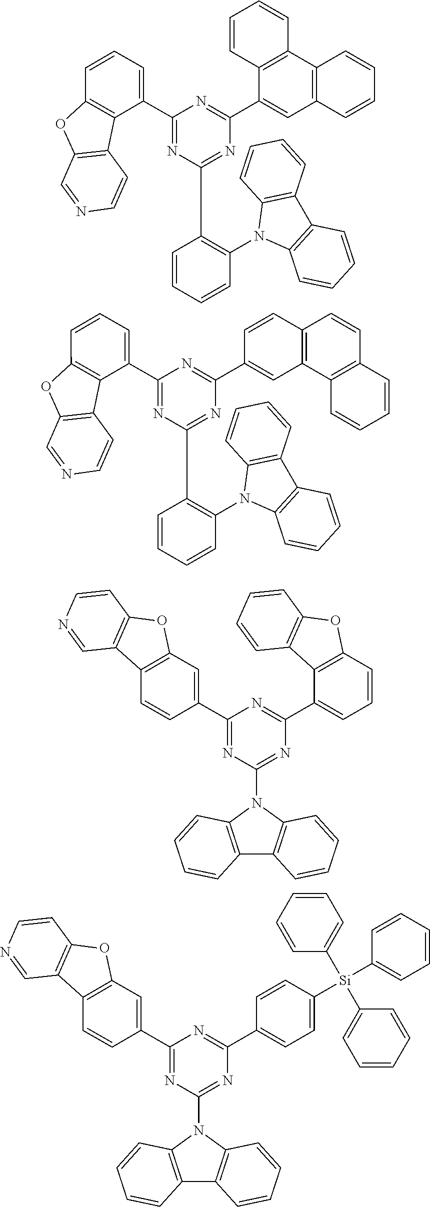

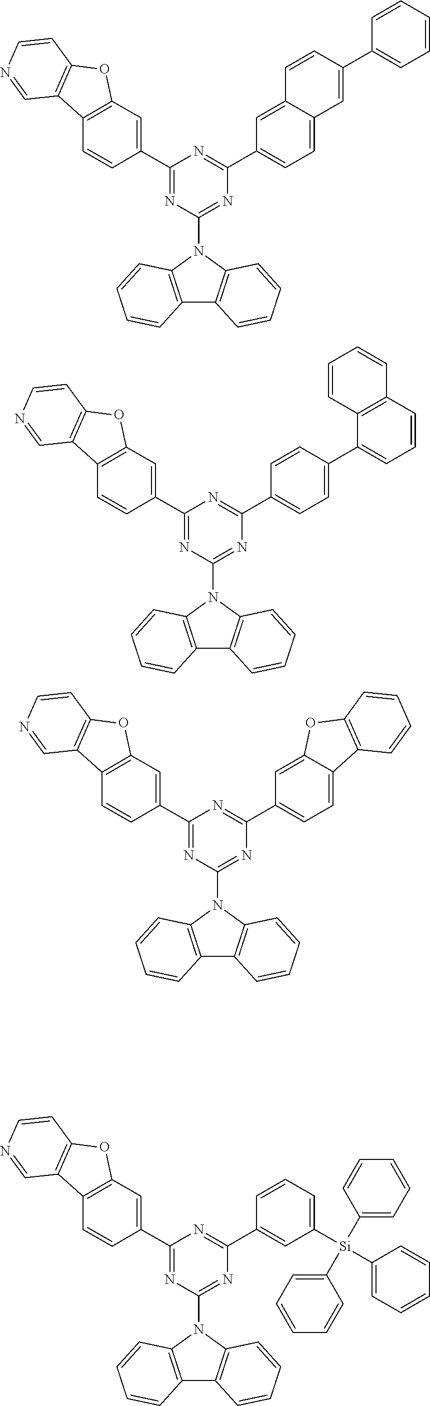 [SMILES: c1ccc(-c2ccc3cc(-c4nc(-c5ccc6c(c5)oc5ccncc56)nc(-n5c6ccccc6c6ccccc65)n4)ccc3c2)cc1 ; c1ccc2c(-c3ccc(-c4nc(-c5ccc6c(c5)oc5ccncc56)nc(-n5c6ccccc6c6ccccc65)n4)cc3)cccc2c1 ; c1ccc2c(c1)oc1cc(-c3nc(-c4ccc5c(c4)oc4ccncc45)nc(-n4c5ccccc5c5ccccc54)n3)ccc12 ; c1ccc([Si](c2ccccc2)(c2ccccc2)c2cccc(-c3nc(-c4ccc5c(c4)oc4ccncc45)nc(-n4c5ccccc5c5ccccc54)n3)c2)cc1]

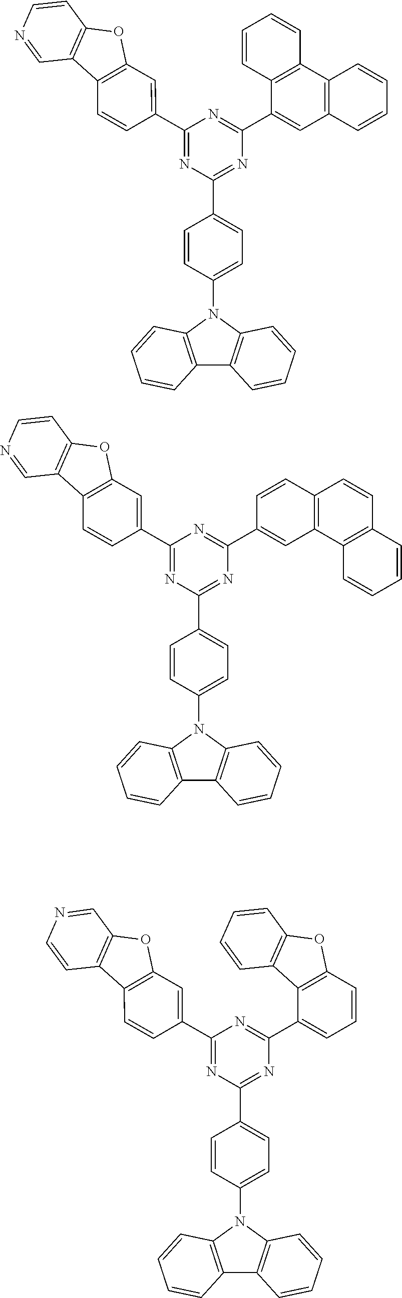 [SMILES: c1ccc2c(c1)cc(-c1nc(-c3ccc(-n4c5ccccc5c5ccccc54)cc3)nc(-c3ccc4c(c3)oc3ccncc34)n1)c1ccccc12 ; c1ccc2c(c1)ccc1ccc(-c3nc(-c4ccc(-n5c6ccccc6c6ccccc65)cc4)nc(-c4ccc5c(c4)oc4ccncc45)n3)cc12 ; c1ccc2c(c1)oc1cccc(-c3nc(-c4ccc(-n5c6ccccc6c6ccccc65)cc4)nc(-c4ccc5c(c4)oc4cnccc45)n3)c12]

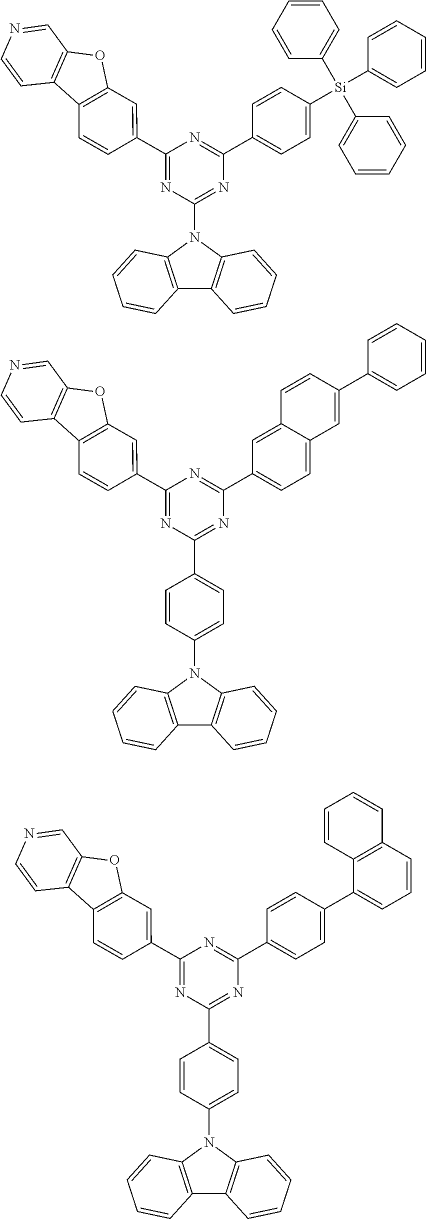 [SMILES: c1ccc([Si](c2ccccc2)(c2ccccc2)c2ccc(-c3nc(-c4ccc5c(c4)oc4cnccc45)nc(-n4c5ccccc5c5ccccc54)n3)cc2)cc1 ; c1ccc(-c2ccc3cc(-c4nc(-c5ccc(-n6c7ccccc7c7ccccc76)cc5)nc(-c5ccc6c(c5)oc5cnccc56)n4)ccc3c2)cc1 ; c1ccc2c(-c3ccc(-c4nc(-c5ccc(-n6c7ccccc7c7ccccc76)cc5)nc(-c5ccc6c(c5)oc5cnccc56)n4)cc3)cccc2c1]

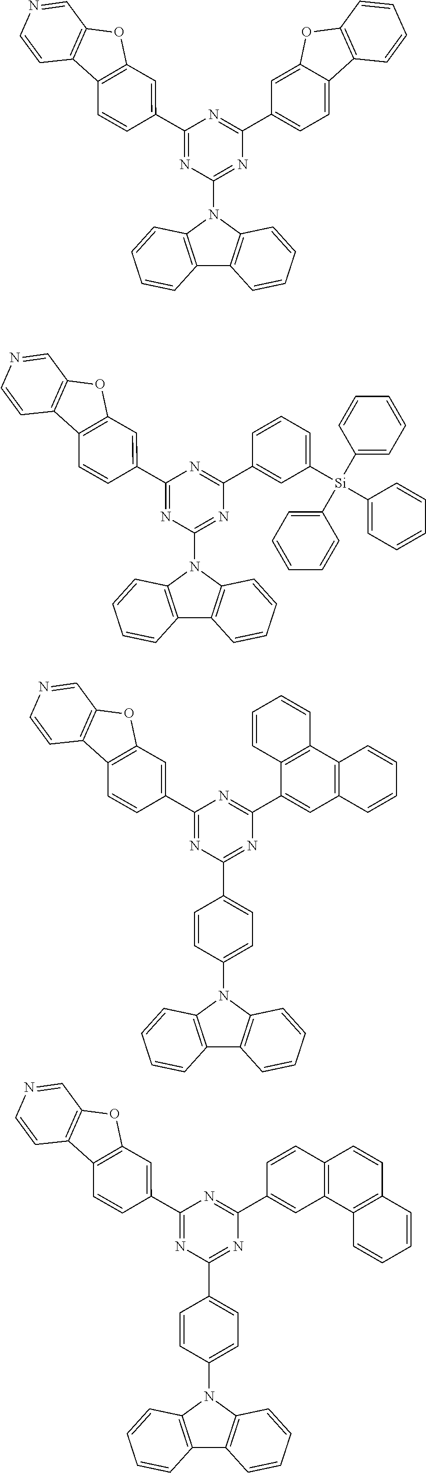 [SMILES: c1ccc2c(c1)oc1cc(-c3nc(-c4ccc5c(c4)oc4cnccc45)nc(-n4c5ccccc5c5ccccc54)n3)ccc12 ; c1ccc([Si](c2ccccc2)(c2ccccc2)c2cccc(-c3nc(-c4ccc5c(c4)oc4cnccc45)nc(-n4c5ccccc5c5ccccc54)n3)c2)cc1 ; c1ccc2c(c1)cc(-c1nc(-c3ccc(-n4c5ccccc5c5ccccc54)cc3)nc(-c3ccc4c(c3)oc3cnccc34)n1)c1ccccc12 ; c1ccc2c(c1)ccc1ccc(-c3nc(-c4ccc(-n5c6ccccc6c6ccccc65)cc4)nc(-c4ccc5c(c4)oc4cnccc45)n3)cc12]

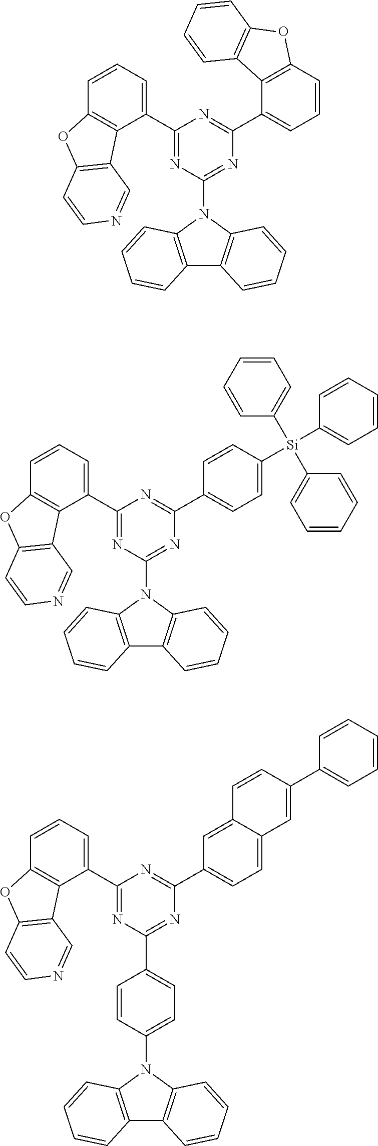 [SMILES: c1ccc2c(c1)oc1cccc(-c3nc(-c4cccc5oc6ccncc6c45)nc(-n4c5ccccc5c5ccccc54)n3)c12 ; c1ccc([Si](c2ccccc2)(c2ccccc2)c2ccc(-c3nc(-c4cccc5oc6ccncc6c45)nc(-n4c5ccccc5c5ccccc54)n3)cc2)cc1 ; c1ccc(-c2ccc3cc(-c4nc(-c5ccc(-n6c7ccccc7c7ccccc76)cc5)nc(-c5cccc6oc7ccncc7c56)n4)ccc3c2)cc1]

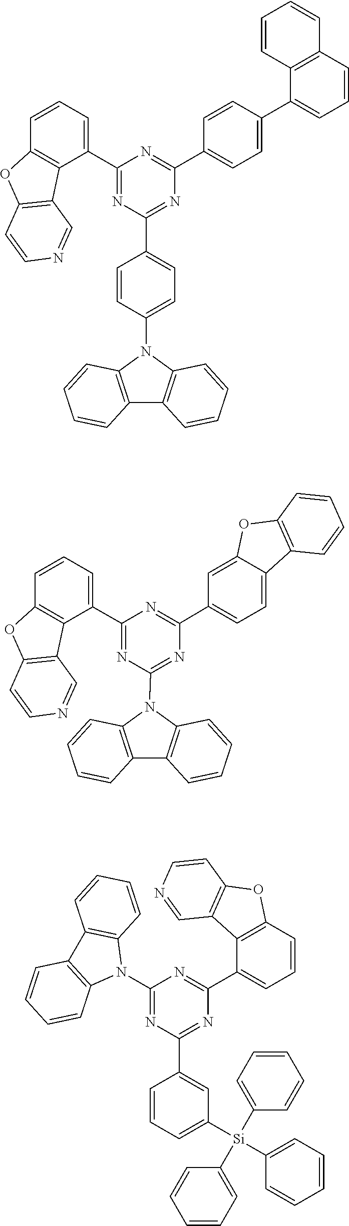 [SMILES: c1ccc2c(-c3ccc(-c4nc(-c5ccc(-n6c7ccccc7c7ccccc76)cc5)nc(-c5cccc6oc7ccncc7c56)n4)cc3)cccc2c1 ; c1ccc2c(c1)oc1cc(-c3nc(-c4cccc5oc6ccncc6c45)nc(-n4c5ccccc5c5ccccc54)n3)ccc12 ; c1ccc([Si](c2ccccc2)(c2ccccc2)c2cccc(-c3nc(-c4cccc5oc6ccncc6c45)nc(-n4c5ccccc5c5ccccc54)n3)c2)cc1]

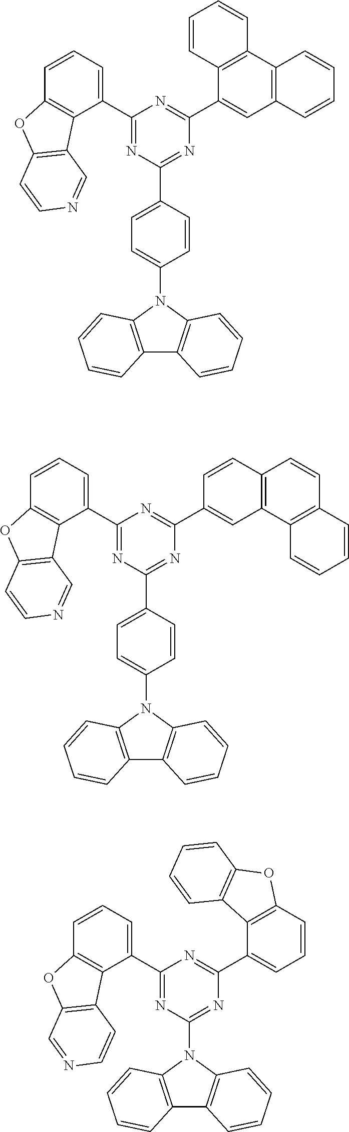 [SMILES: c1ccc2c(c1)cc(-c1nc(-c3ccc(-n4c5ccccc5c5ccccc54)cc3)nc(-c3cccc4oc5ccncc5c34)n1)c1ccccc12 ; c1ccc2c(c1)ccc1ccc(-c3nc(-c4ccc(-n5c6ccccc6c6ccccc65)cc4)nc(-c4cccc5oc6ccncc6c45)n3)cc12 ; c1ccc2c(c1)oc1cccc(-c3nc(-c4cccc5oc6cnccc6c45)nc(-n4c5ccccc5c5ccccc54)n3)c12]

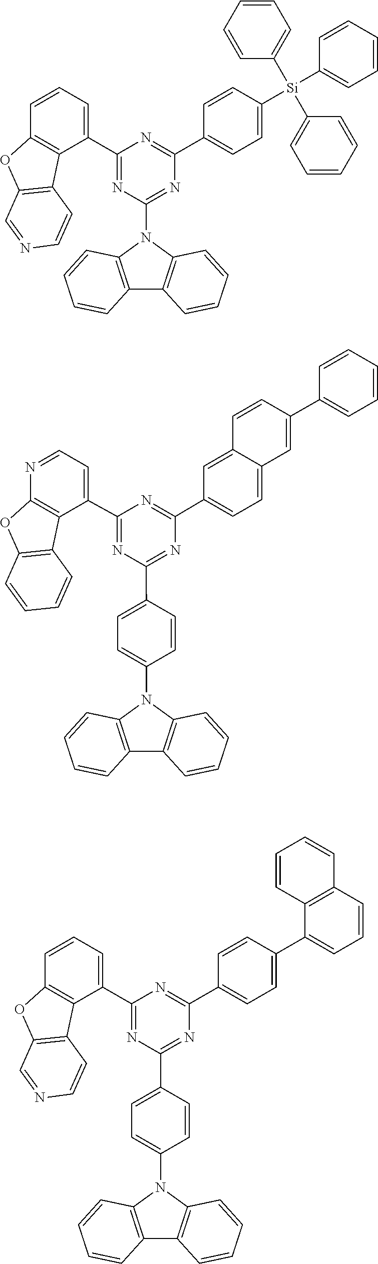 [SMILES: c1ccc([Si](c2ccccc2)(c2ccccc2)c2ccc(-c3nc(-c4cccc5oc6cnccc6c45)nc(-n4c5ccccc5c5ccccc54)n3)cc2)cc1 ; c1ccc(-c2ccc3cc(-c4nc(-c5ccc(-n6c7ccccc7c7ccccc76)cc5)nc(-c5ccnc6oc7ccccc7c56)n4)ccc3c2)cc1 ; c1ccc2c(-c3ccc(-c4nc(-c5ccc(-n6c7ccccc7c7ccccc76)cc5)nc(-c5cccc6oc7cnccc7c56)n4)cc3)cccc2c1]

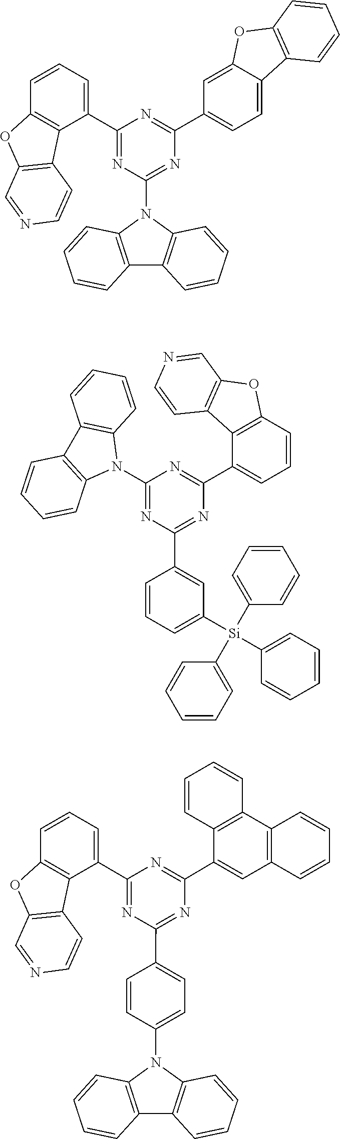

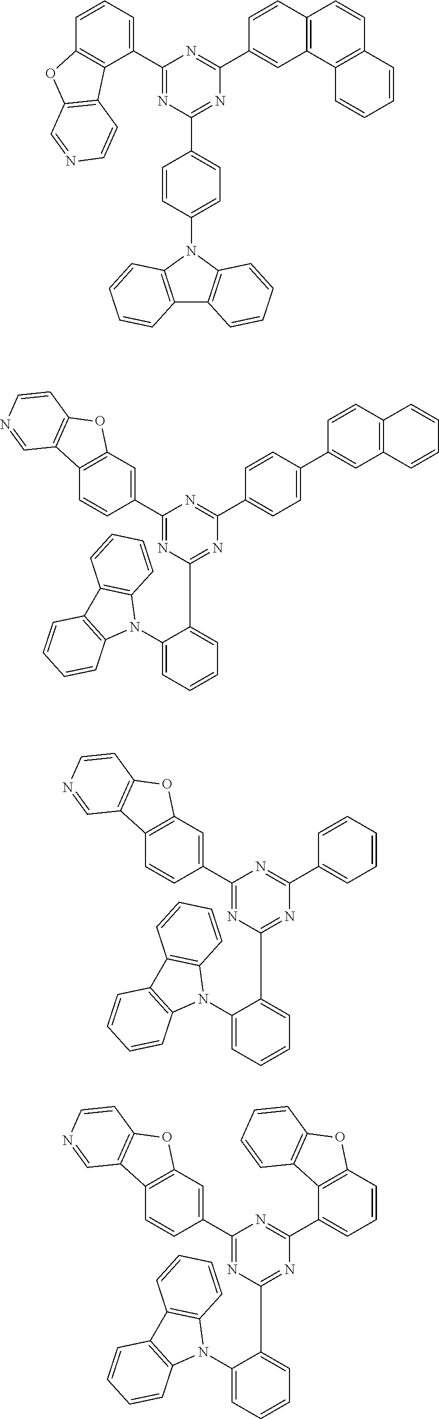

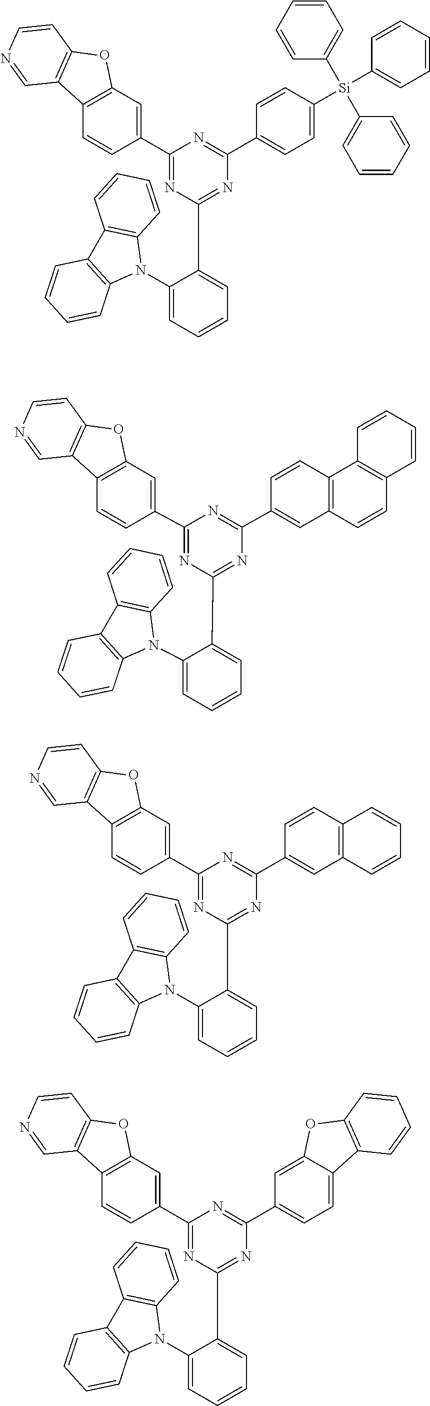 [SMILES: c1ccc([Si](c2ccccc2)(c2ccccc2)c2ccc(-c3nc(-c4ccc5c(c4)oc4ccncc45)nc(-c4ccccc4-n4c5ccccc5c5ccccc54)n3)cc2)cc1 ; c1ccc(-n2c3ccccc3c3ccccc32)c(-c2nc(-c3ccc4c(ccc5ccccc54)c3)nc(-c3ccc4c(c3)oc3ccncc34)n2)c1 ; c1ccc(-n2c3ccccc3c3ccccc32)c(-c2nc(-c3ccc4ccccc4c3)nc(-c3ccc4c(c3)oc3ccncc34)n2)c1 ; c1ccc(-n2c3ccccc3c3ccccc32)c(-c2nc(-c3ccc4c(c3)oc3ccccc34)nc(-c3ccc4c(c3)oc3ccncc34)n2)c1]

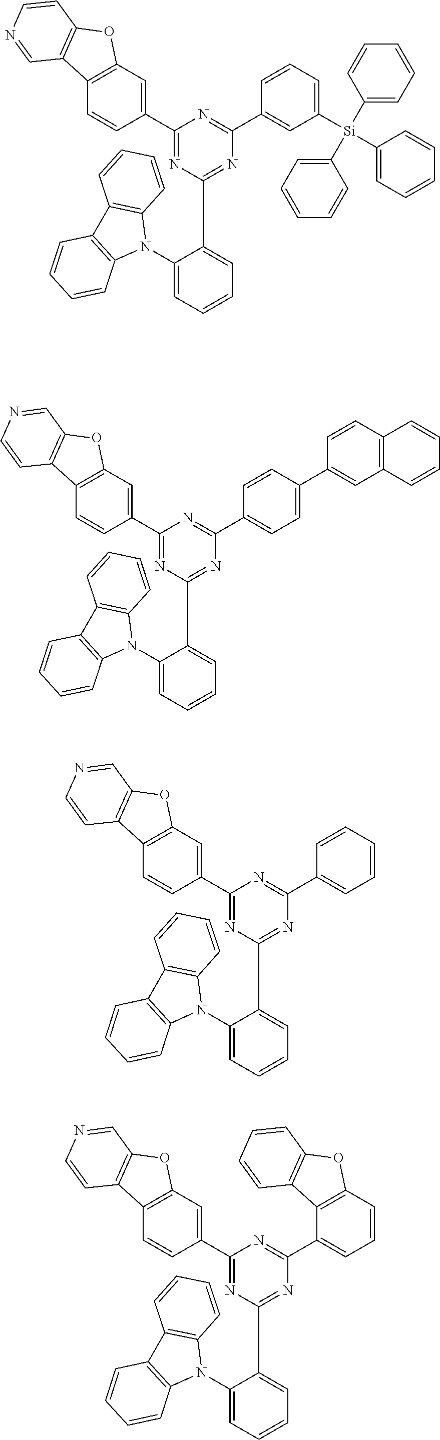 [SMILES: c1ccc([Si](c2ccccc2)(c2ccccc2)c2cccc(-c3nc(-c4ccc5c(c4)oc4ccncc45)nc(-c4ccccc4-n4c5ccccc5c5ccccc54)n3)c2)cc1 ; c1ccc(-n2c3ccccc3c3ccccc32)c(-c2nc(-c3ccc(-c4ccc5ccccc5c4)cc3)nc(-c3ccc4c(c3)oc3cnccc34)n2)c1 ; c1ccc(-c2nc(-c3ccc4c(c3)oc3cnccc34)nc(-c3ccccc3-n3c4ccccc4c4ccccc43)n2)cc1 ; c1ccc(-n2c3ccccc3c3ccccc32)c(-c2nc(-c3ccc4c(c3)oc3cnccc34)nc(-c3cccc4oc5ccccc5c34)n2)c1]

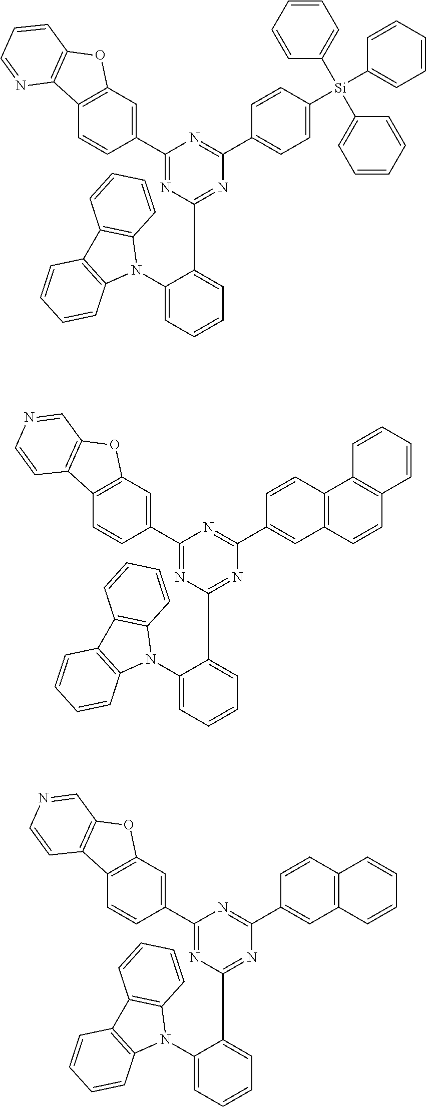 [SMILES: c1ccc([Si](c2ccccc2)(c2ccccc2)c2ccc(-c3nc(-c4ccc5c(c4)oc4cccnc45)nc(-c4ccccc4-n4c5ccccc5c5ccccc54)n3)cc2)cc1 ; c1ccc(-n2c3ccccc3c3ccccc32)c(-c2nc(-c3ccc4c(ccc5ccccc54)c3)nc(-c3ccc4c(c3)oc3cnccc34)n2)c1 ; c1ccc(-n2c3ccccc3c3ccccc32)c(-c2nc(-c3ccc4ccccc4c3)nc(-c3ccc4c(c3)oc3cnccc34)n2)c1]

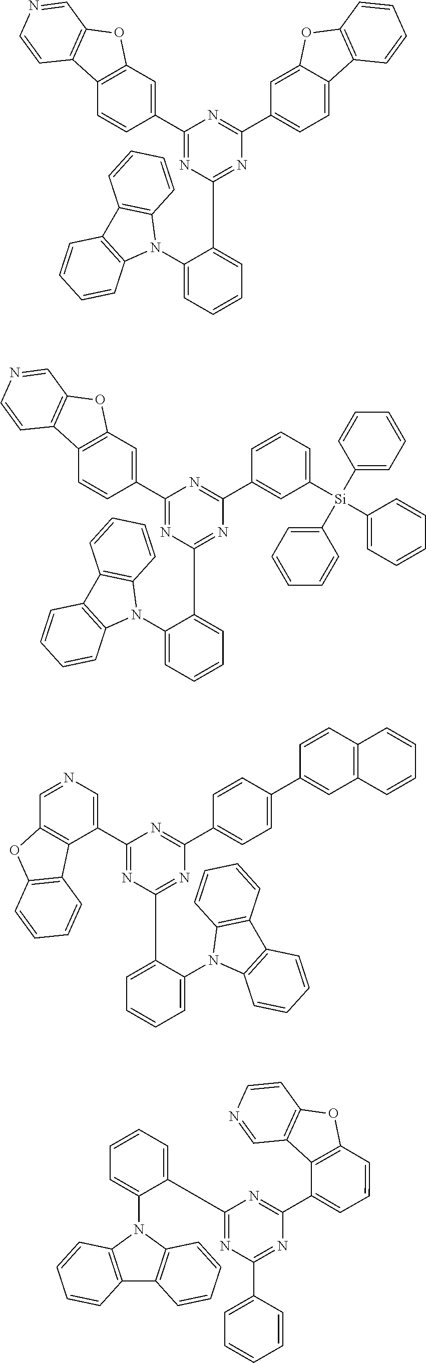 [SMILES: c1ccc(-n2c3ccccc3c3ccccc32)c(-c2nc(-c3ccc4c(c3)oc3ccccc34)nc(-c3ccc4c(c3)oc3cnccc34)n2)c1 ; c1ccc([Si](c2ccccc2)(c2ccccc2)c2cccc(-c3nc(-c4ccc5c(c4)oc4cnccc45)nc(-c4ccccc4-n4c5ccccc5c5ccccc54)n3)c2)cc1 ; c1ccc(-n2c3ccccc3c3ccccc32)c(-c2nc(-c3ccc(-c4ccc5ccccc5c4)cc3)nc(-c3cncc4oc5ccccc5c34)n2)c1 ; c1ccc(-c2nc(-c3ccccc3-n3c4ccccc4c4ccccc43)nc(-c3cccc4oc5ccncc5c34)n2)cc1]

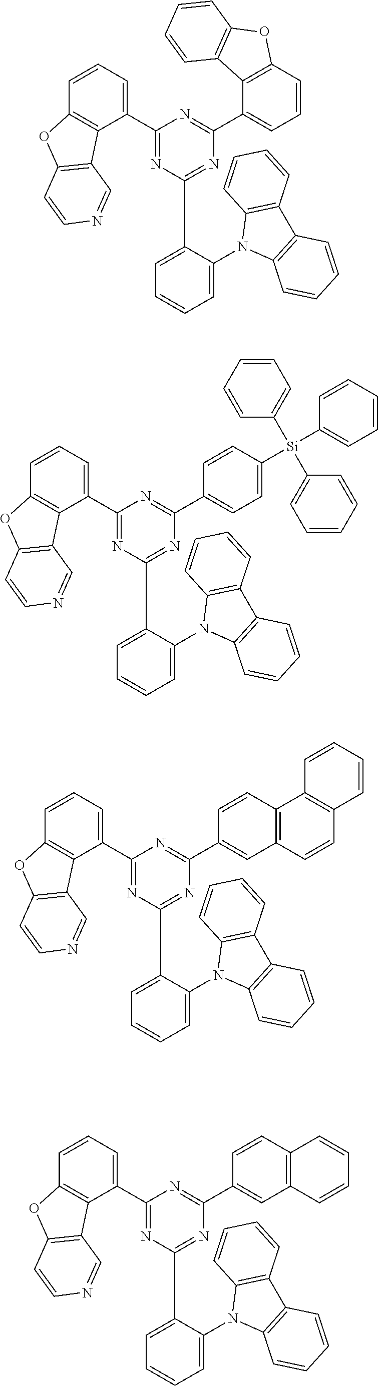 [SMILES: c1ccc(-n2c3ccccc3c3ccccc32)c(-c2nc(-c3cccc4oc5ccccc5c34)nc(-c3cccc4oc5ccncc5c34)n2)c1 ; c1ccc([Si](c2ccccc2)(c2ccccc2)c2ccc(-c3nc(-c4ccccc4-n4c5ccccc5c5ccccc54)nc(-c4cccc5oc6ccncc6c45)n3)cc2)cc1 ; c1ccc(-n2c3ccccc3c3ccccc32)c(-c2nc(-c3ccc4c(ccc5ccccc54)c3)nc(-c3cccc4oc5ccncc5c34)n2)c1 ; c1ccc(-n2c3ccccc3c3ccccc32)c(-c2nc(-c3ccc4ccccc4c3)nc(-c3cccc4oc5ccncc5c34)n2)c1]

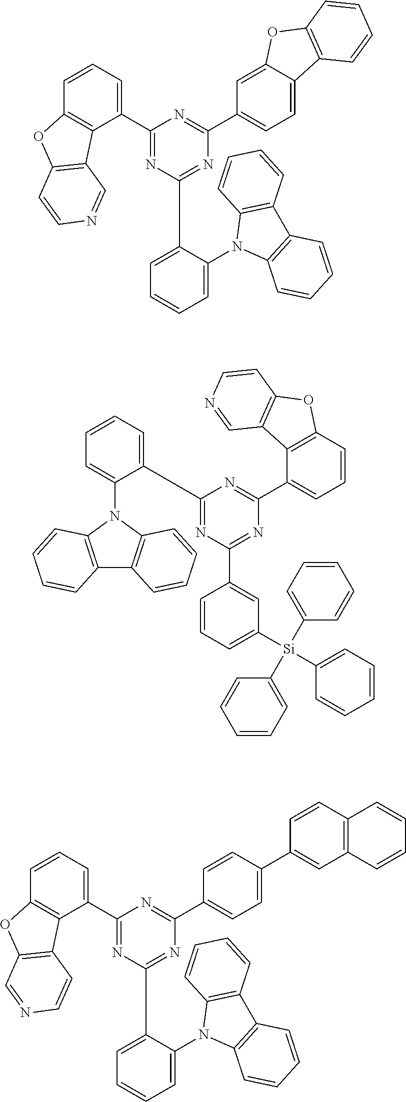 [SMILES: c1ccc(-n2c3ccccc3c3ccccc32)c(-c2nc(-c3ccc4c(c3)oc3ccccc34)nc(-c3cccc4oc5ccncc5c34)n2)c1 ; c1ccc([Si](c2ccccc2)(c2ccccc2)c2cccc(-c3nc(-c4ccccc4-n4c5ccccc5c5ccccc54)nc(-c4cccc5oc6ccncc6c45)n3)c2)cc1 ; c1ccc(-n2c3ccccc3c3ccccc32)c(-c2nc(-c3ccc(-c4ccc5ccccc5c4)cc3)nc(-c3cccc4oc5cnccc5c34)n2)c1]

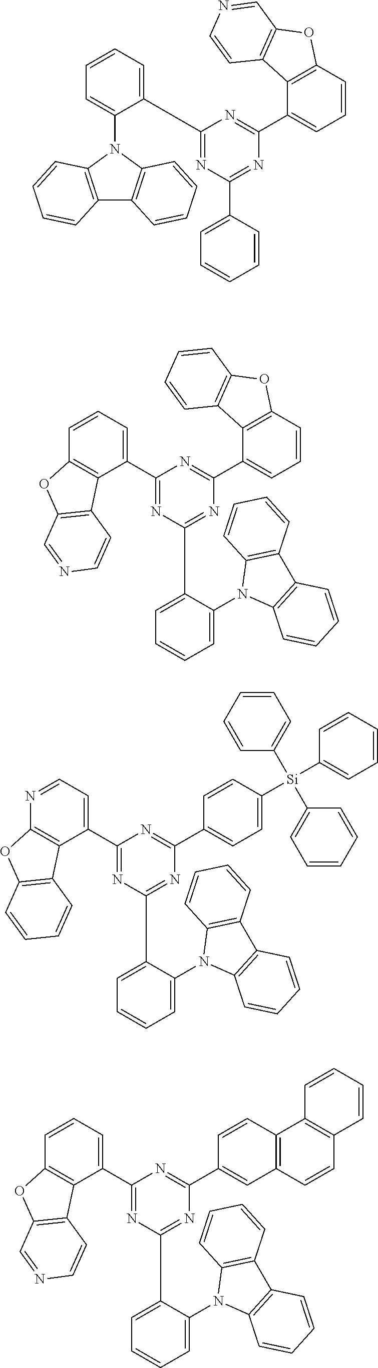 [SMILES: c1ccc(-c2nc(-c3ccccc3-n3c4ccccc4c4ccccc43)nc(-c3cccc4oc5cnccc5c34)n2)cc1 ; c1ccc(-n2c3ccccc3c3ccccc32)c(-c2nc(-c3cccc4oc5ccccc5c34)nc(-c3cccc4oc5cnccc5c34)n2)c1 ; c1ccc([Si](c2ccccc2)(c2ccccc2)c2ccc(-c3nc(-c4ccccc4-n4c5ccccc5c5ccccc54)nc(-c4ccnc5oc6ccccc6c45)n3)cc2)cc1 ; c1ccc(-n2c3ccccc3c3ccccc32)c(-c2nc(-c3ccc4c(ccc5ccccc54)c3)nc(-c3cccc4oc5cnccc5c34)n2)c1]

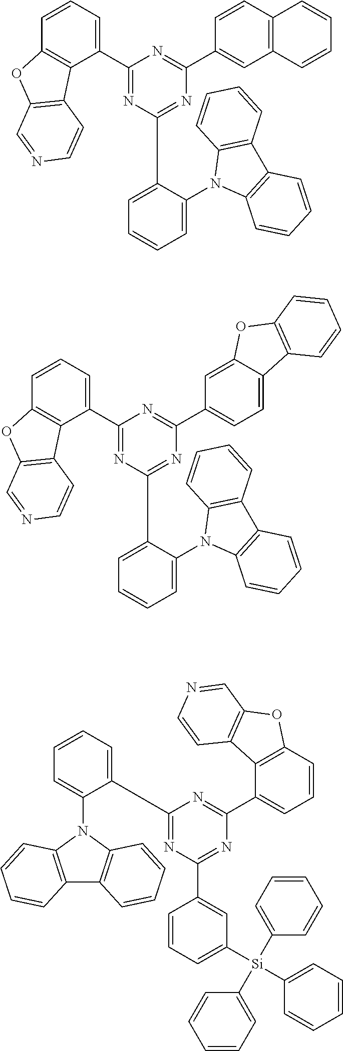 [SMILES: c1ccc(-n2c3ccccc3c3ccccc32)c(-c2nc(-c3ccc4ccccc4c3)nc(-c3cccc4oc5cnccc5c34)n2)c1 ; c1ccc(-n2c3ccccc3c3ccccc32)c(-c2nc(-c3ccc4c(c3)oc3ccccc34)nc(-c3cccc4oc5cnccc5c34)n2)c1 ; c1ccc([Si](c2ccccc2)(c2ccccc2)c2cccc(-c3nc(-c4ccccc4-n4c5ccccc5c5ccccc54)nc(-c4cccc5oc6cnccc6c45)n3)c2)cc1]

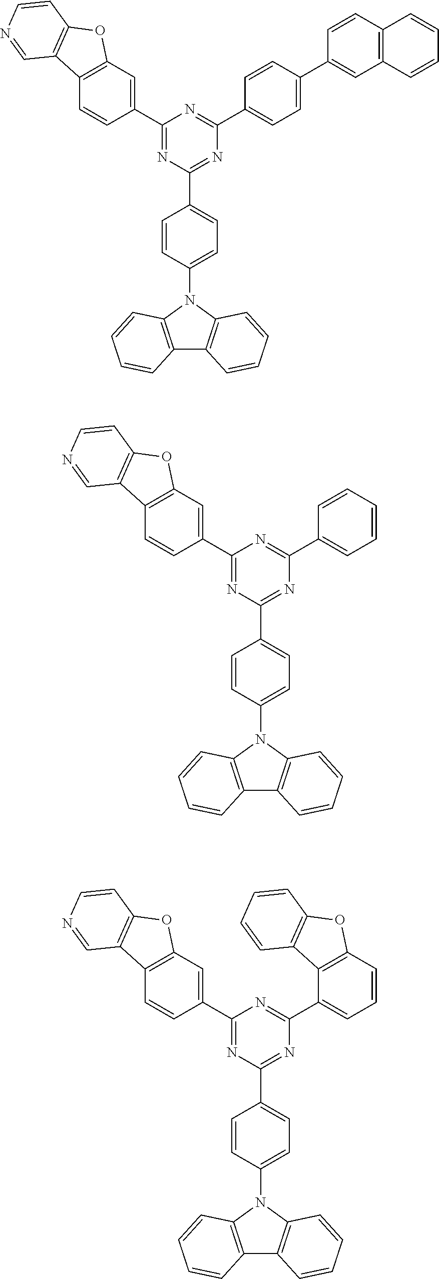 [SMILES: c1ccc2cc(-c3ccc(-c4nc(-c5ccc(-n6c7ccccc7c7ccccc76)cc5)nc(-c5ccc6c(c5)oc5ccncc56)n4)cc3)ccc2c1 ; c1ccc(-c2nc(-c3ccc(-n4c5ccccc5c5ccccc54)cc3)nc(-c3ccc4c(c3)oc3ccncc34)n2)cc1 ; c1ccc2c(c1)oc1cccc(-c3nc(-c4ccc(-n5c6ccccc6c6ccccc65)cc4)nc(-c4ccc5c(c4)oc4ccncc45)n3)c12]

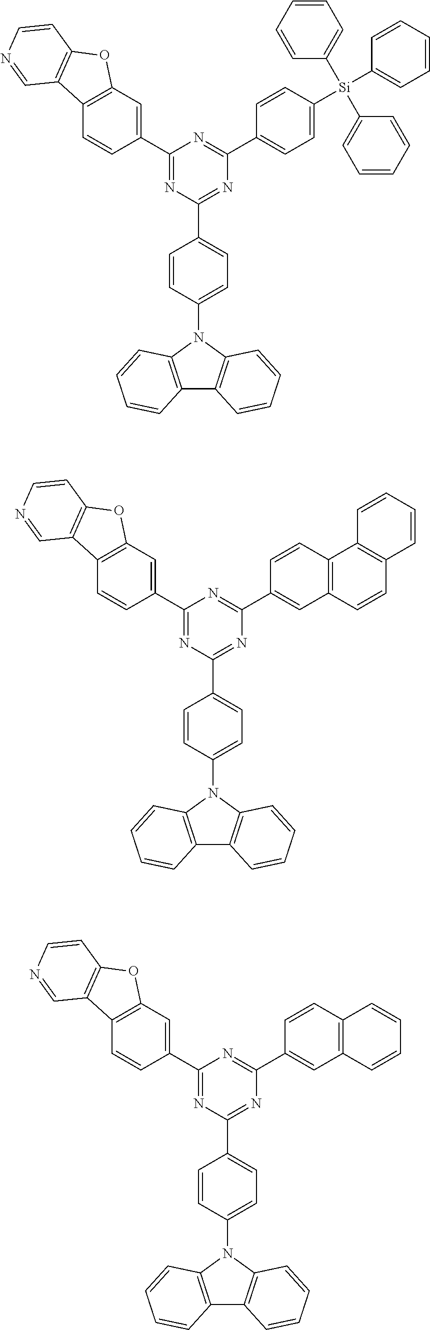 [SMILES: c1ccc([Si](c2ccccc2)(c2ccccc2)c2ccc(-c3nc(-c4ccc(-n5c6ccccc6c6ccccc65)cc4)nc(-c4ccc5c(c4)oc4ccncc45)n3)cc2)cc1 ; c1ccc2c(c1)ccc1cc(-c3nc(-c4ccc(-n5c6ccccc6c6ccccc65)cc4)nc(-c4ccc5c(c4)oc4ccncc45)n3)ccc12 ; c1ccc2cc(-c3nc(-c4ccc(-n5c6ccccc6c6ccccc65)cc4)nc(-c4ccc5c(c4)oc4ccncc45)n3)ccc2c1]

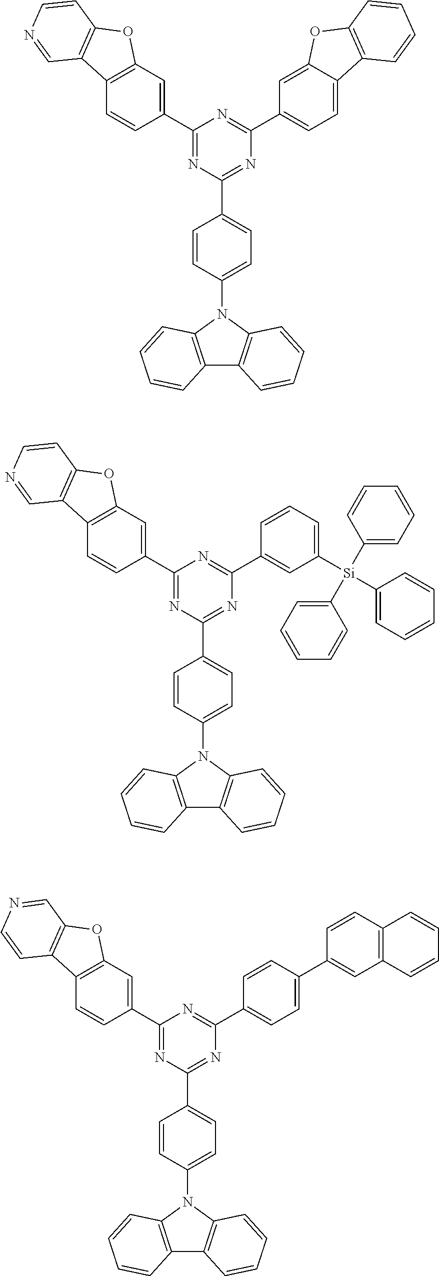 [SMILES: c1ccc2c(c1)oc1cc(-c3nc(-c4ccc(-n5c6ccccc6c6ccccc65)cc4)nc(-c4ccc5c(c4)oc4ccncc45)n3)ccc12 ; c1ccc([Si](c2ccccc2)(c2ccccc2)c2cccc(-c3nc(-c4ccc(-n5c6ccccc6c6ccccc65)cc4)nc(-c4ccc5c(c4)oc4ccncc45)n3)c2)cc1 ; c1ccc2cc(-c3ccc(-c4nc(-c5ccc(-n6c7ccccc7c7ccccc76)cc5)nc(-c5ccc6c(c5)oc5cnccc56)n4)cc3)ccc2c1]

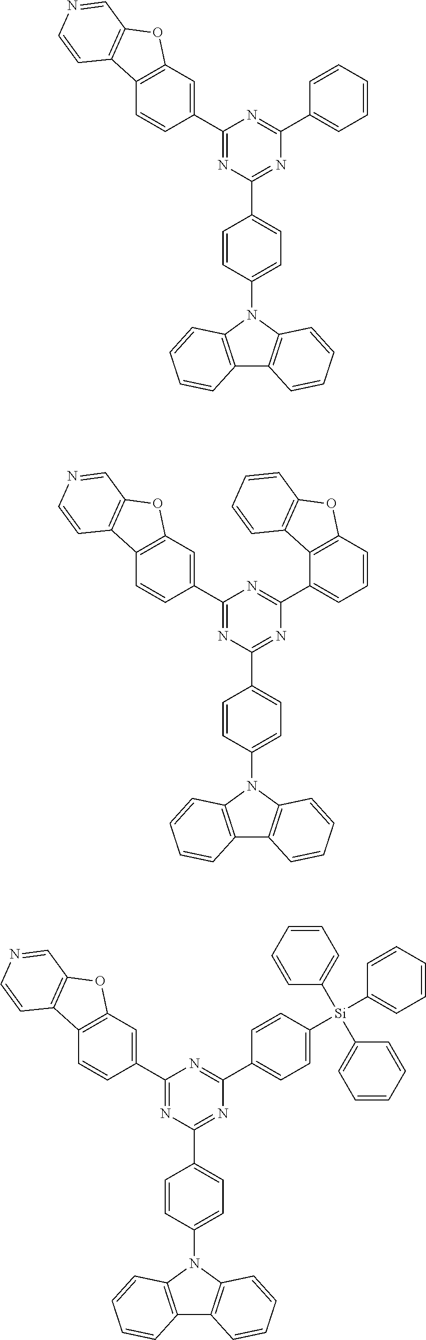 [SMILES: c1ccc(-c2nc(-c3ccc(-n4c5ccccc5c5ccccc54)cc3)nc(-c3ccc4c(c3)oc3cnccc34)n2)cc1 ; c1ccc2c(c1)oc1cccc(-c3nc(-c4ccc(-n5c6ccccc6c6ccccc65)cc4)nc(-c4ccc5c(c4)oc4cnccc45)n3)c12 ; c1ccc([Si](c2ccccc2)(c2ccccc2)c2ccc(-c3nc(-c4ccc(-n5c6ccccc6c6ccccc65)cc4)nc(-c4ccc5c(c4)oc4cnccc45)n3)cc2)cc1]

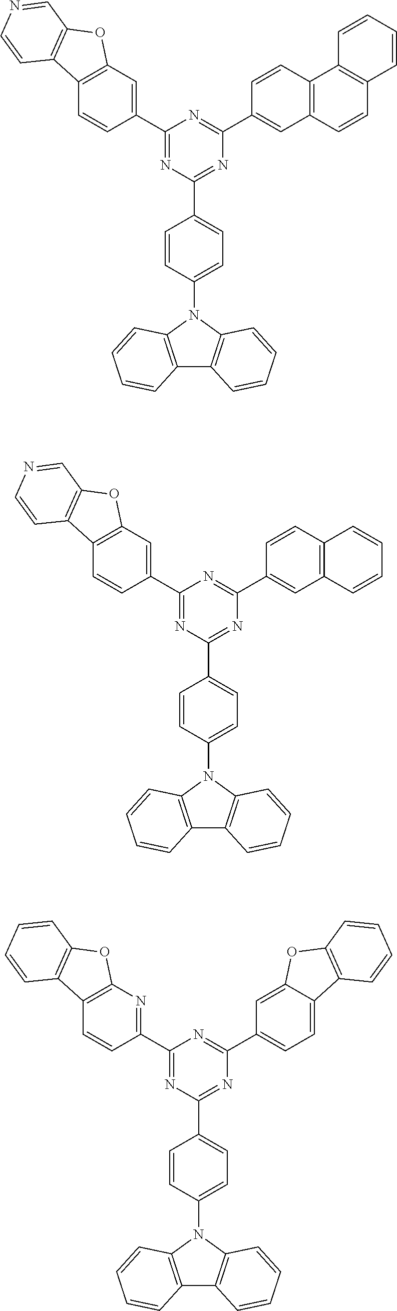 [SMILES: c1ccc2c(c1)ccc1cc(-c3nc(-c4ccc(-n5c6ccccc6c6ccccc65)cc4)nc(-c4ccc5c(c4)oc4cnccc45)n3)ccc12 ; c1ccc2cc(-c3nc(-c4ccc(-n5c6ccccc6c6ccccc65)cc4)nc(-c4ccc5c(c4)oc4cnccc45)n3)ccc2c1 ; c1ccc2c(c1)oc1cc(-c3nc(-c4ccc(-n5c6ccccc6c6ccccc65)cc4)nc(-c4ccc5c(n4)oc4ccccc45)n3)ccc12]

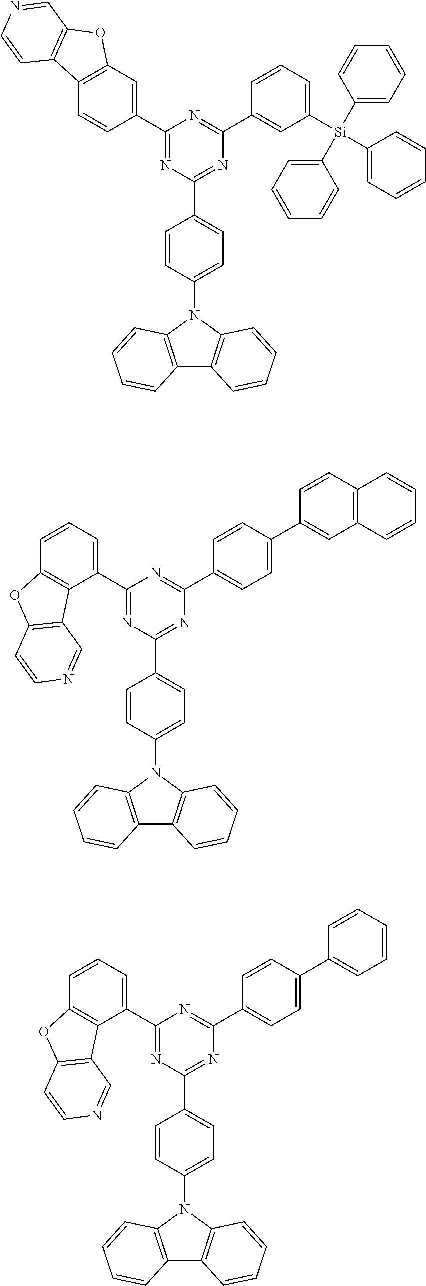 [SMILES: c1ccc([Si](c2ccccc2)(c2ccccc2)c2cccc(-c3nc(-c4ccc(-n5c6ccccc6c6ccccc65)cc4)nc(-c4ccc5c(c4)oc4cnccc45)n3)c2)cc1 ; c1ccc2cc(-c3ccc(-c4nc(-c5ccc(-n6c7ccccc7c7ccccc76)cc5)nc(-c5cccc6oc7ccncc7c56)n4)cc3)ccc2c1 ; c1ccc(-c2ccc(-c3nc(-c4ccc(-n5c6ccccc6c6ccccc65)cc4)nc(-c4cccc5oc6ccncc6c45)n3)cc2)cc1]

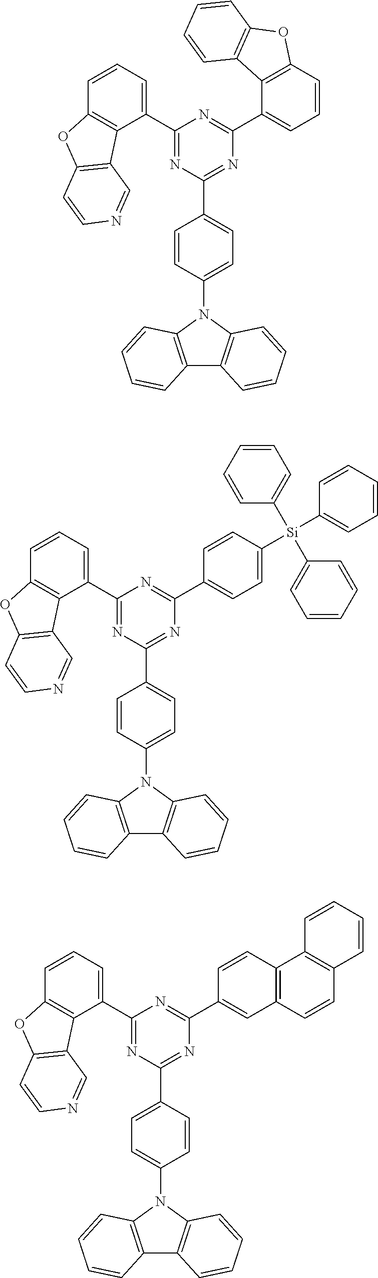 [SMILES: c1ccc2c(c1)oc1cccc(-c3nc(-c4ccc(-n5c6ccccc6c6ccccc65)cc4)nc(-c4cccc5oc6ccncc6c45)n3)c12 ; c1ccc([Si](c2ccccc2)(c2ccccc2)c2ccc(-c3nc(-c4ccc(-n5c6ccccc6c6ccccc65)cc4)nc(-c4cccc5oc6ccncc6c45)n3)cc2)cc1 ; c1ccc2c(c1)ccc1cc(-c3nc(-c4ccc(-n5c6ccccc6c6ccccc65)cc4)nc(-c4cccc5oc6ccncc6c45)n3)ccc12]

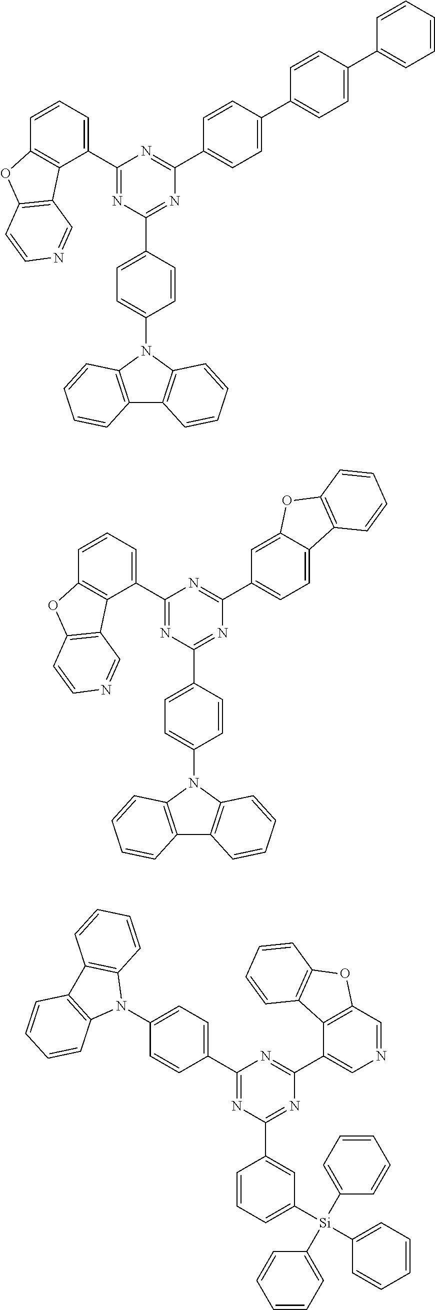 [SMILES: c1ccc(-c2ccc(-c3ccc(-c4nc(-c5ccc(-n6c7ccccc7c7ccccc76)cc5)nc(-c5cccc6oc7ccncc7c56)n4)cc3)cc2)cc1 ; c1ccc2c(c1)oc1cc(-c3nc(-c4ccc(-n5c6ccccc6c6ccccc65)cc4)nc(-c4cccc5oc6ccncc6c45)n3)ccc12 ; c1ccc([Si](c2ccccc2)(c2ccccc2)c2cccc(-c3nc(-c4ccc(-n5c6ccccc6c6ccccc65)cc4)nc(-c4cncc5oc6ccccc6c45)n3)c2)cc1]

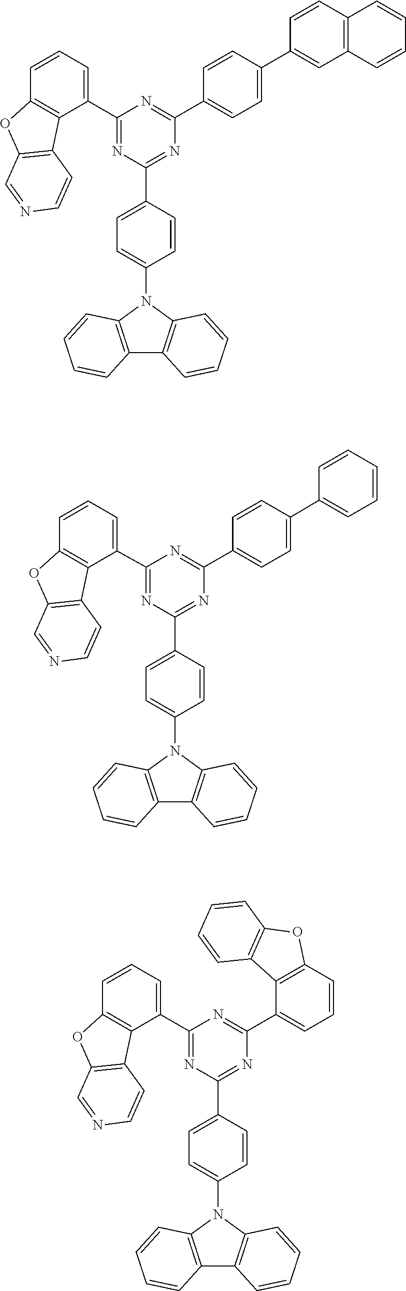 [SMILES: c1ccc2cc(-c3ccc(-c4nc(-c5ccc(-n6c7ccccc7c7ccccc76)cc5)nc(-c5cccc6oc7cnccc7c56)n4)cc3)ccc2c1 ; c1ccc(-c2ccc(-c3nc(-c4ccc(-n5c6ccccc6c6ccccc65)cc4)nc(-c4cccc5oc6cnccc6c45)n3)cc2)cc1 ; c1ccc2c(c1)oc1cccc(-c3nc(-c4ccc(-n5c6ccccc6c6ccccc65)cc4)nc(-c4cccc5oc6cnccc6c45)n3)c12]

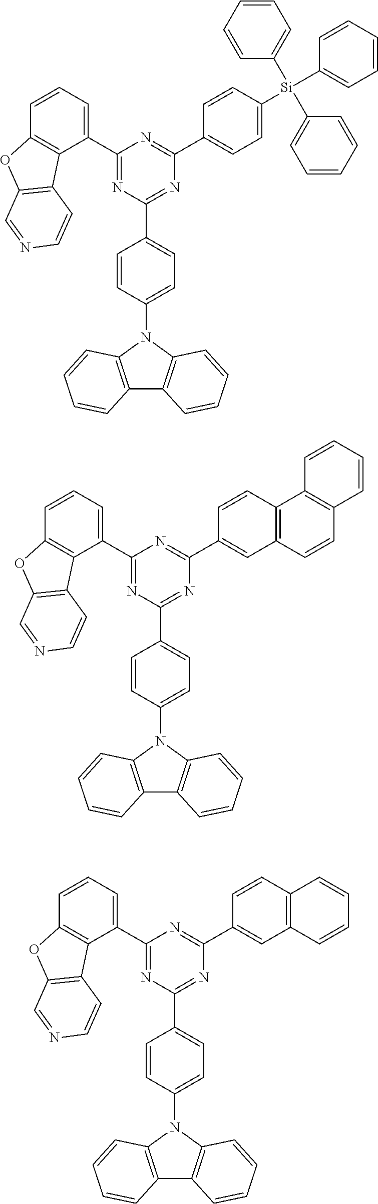 [SMILES: c1ccc([Si](c2ccccc2)(c2ccccc2)c2ccc(-c3nc(-c4ccc(-n5c6ccccc6c6ccccc65)cc4)nc(-c4cccc5oc6cnccc6c45)n3)cc2)cc1 ; c1ccc2c(c1)ccc1cc(-c3nc(-c4ccc(-n5c6ccccc6c6ccccc65)cc4)nc(-c4cccc5oc6cnccc6c45)n3)ccc12 ; c1ccc2cc(-c3nc(-c4ccc(-n5c6ccccc6c6ccccc65)cc4)nc(-c4cccc5oc6cnccc6c45)n3)ccc2c1]

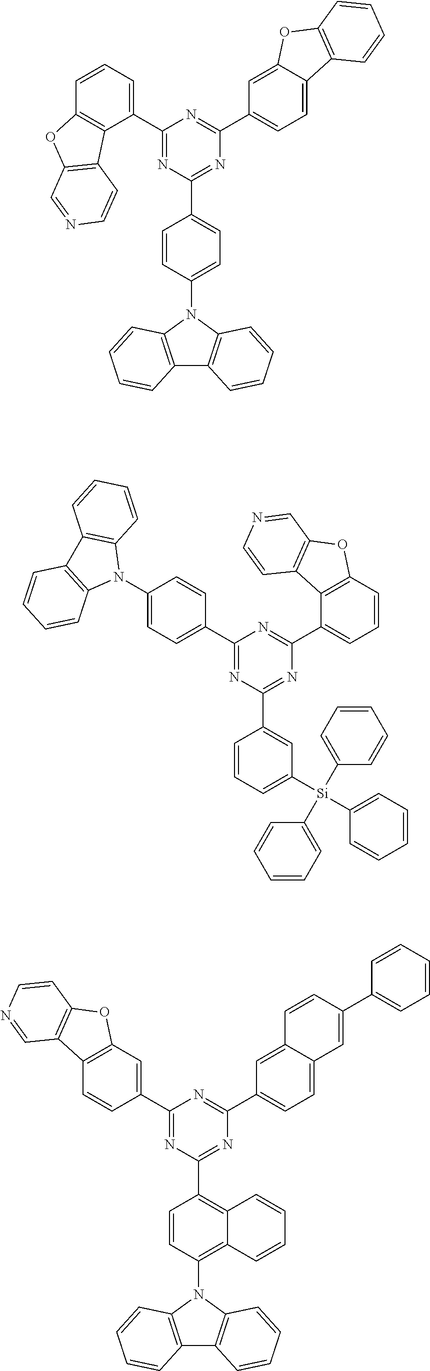 [SMILES: c1ccc2c(c1)oc1cc(-c3nc(-c4ccc(-n5c6ccccc6c6ccccc65)cc4)nc(-c4cccc5oc6cnccc6c45)n3)ccc12 ; c1ccc([Si](c2ccccc2)(c2ccccc2)c2cccc(-c3nc(-c4ccc(-n5c6ccccc6c6ccccc65)cc4)nc(-c4cccc5oc6cnccc6c45)n3)c2)cc1 ; c1ccc(-c2ccc3cc(-c4nc(-c5ccc6c(c5)oc5ccncc56)nc(-c5ccc(-n6c7ccccc7c7ccccc76)c6ccccc56)n4)ccc3c2)cc1]

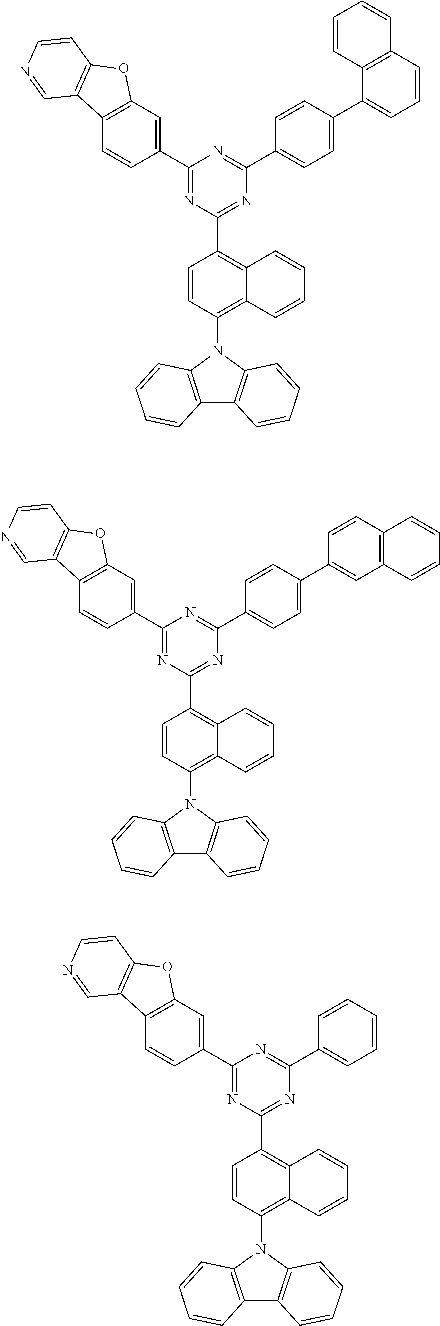 [SMILES: c1ccc2c(-c3ccc(-c4nc(-c5ccc6c(c5)oc5ccncc56)nc(-c5ccc(-n6c7ccccc7c7ccccc76)c6ccccc56)n4)cc3)cccc2c1 ; c1ccc2cc(-c3ccc(-c4nc(-c5ccc6c(c5)oc5ccncc56)nc(-c5ccc(-n6c7ccccc7c7ccccc76)c6ccccc56)n4)cc3)ccc2c1 ; c1ccc(-c2nc(-c3ccc4c(c3)oc3ccncc34)nc(-c3ccc(-n4c5ccccc5c5ccccc54)c4ccccc34)n2)cc1]

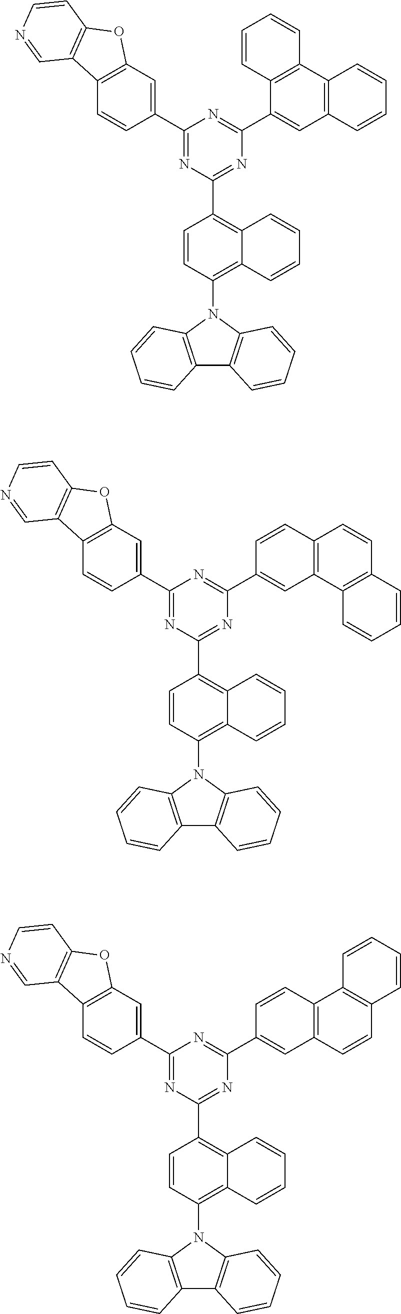 [SMILES: c1ccc2c(c1)cc(-c1nc(-c3ccc4c(c3)oc3ccncc34)nc(-c3ccc(-n4c5ccccc5c5ccccc54)c4ccccc34)n1)c1ccccc12 ; c1ccc2c(c1)ccc1ccc(-c3nc(-c4ccc5c(c4)oc4ccncc45)nc(-c4ccc(-n5c6ccccc6c6ccccc65)c5ccccc45)n3)cc12 ; c1ccc2c(c1)ccc1cc(-c3nc(-c4ccc5c(c4)oc4ccncc45)nc(-c4ccc(-n5c6ccccc6c6ccccc65)c5ccccc45)n3)ccc12]

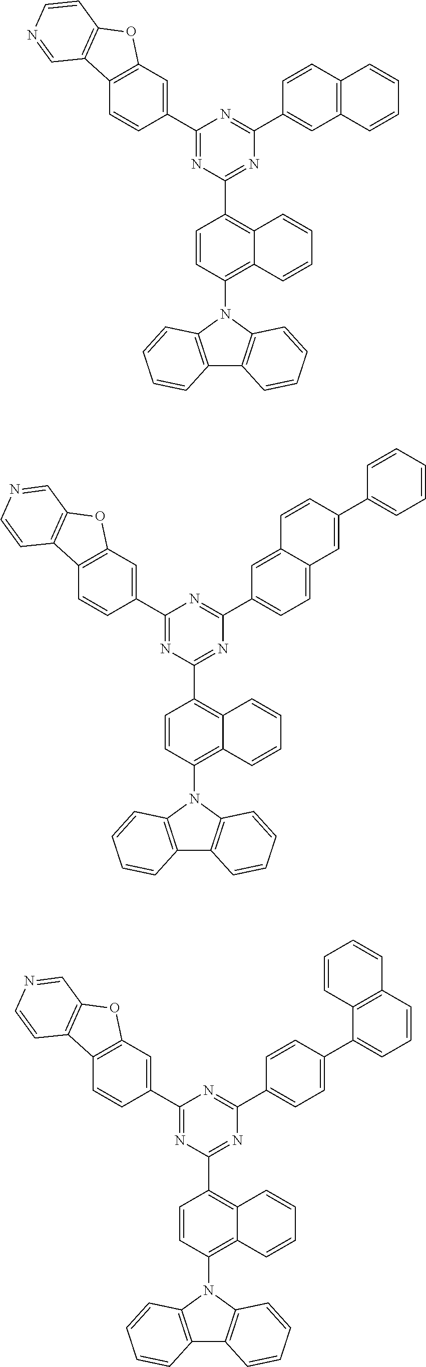 [SMILES: c1ccc2cc(-c3nc(-c4ccc5c(c4)oc4ccncc45)nc(-c4ccc(-n5c6ccccc6c6ccccc65)c5ccccc45)n3)ccc2c1 ; c1ccc(-c2ccc3cc(-c4nc(-c5ccc6c(c5)oc5cnccc56)nc(-c5ccc(-n6c7ccccc7c7ccccc76)c6ccccc56)n4)ccc3c2)cc1 ; c1ccc2c(-c3ccc(-c4nc(-c5ccc6c(c5)oc5cnccc56)nc(-c5ccc(-n6c7ccccc7c7ccccc76)c6ccccc56)n4)cc3)cccc2c1]

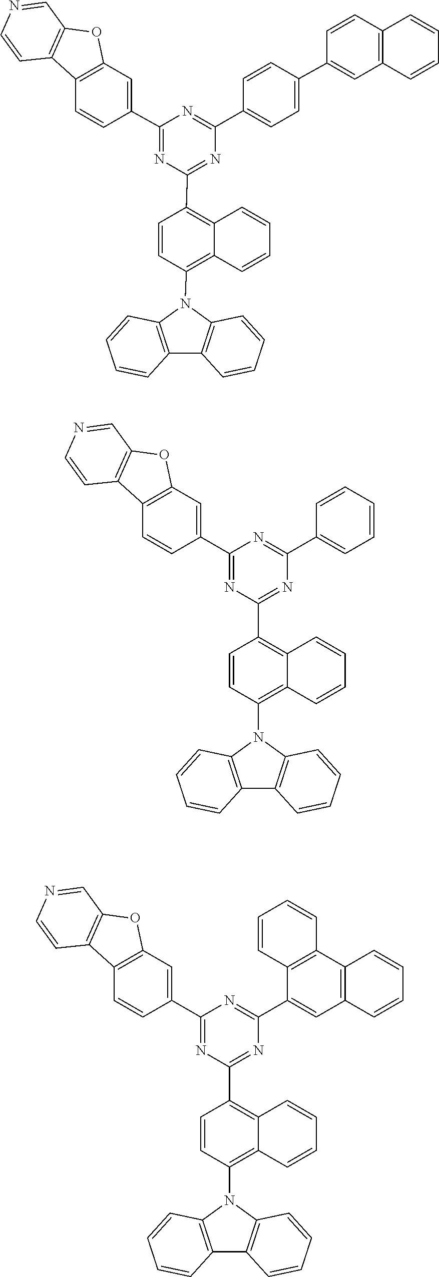 [SMILES: c1ccc2cc(-c3ccc(-c4nc(-c5ccc6c(c5)oc5cnccc56)nc(-c5ccc(-n6c7ccccc7c7ccccc76)c6ccccc56)n4)cc3)ccc2c1 ; c1ccc(-c2nc(-c3ccc4c(c3)oc3cnccc34)nc(-c3ccc(-n4c5ccccc5c5ccccc54)c4ccccc34)n2)cc1 ; c1ccc2c(c1)cc(-c1nc(-c3ccc4c(c3)oc3cnccc34)nc(-c3ccc(-n4c5ccccc5c5ccccc54)c4ccccc34)n1)c1ccccc12]

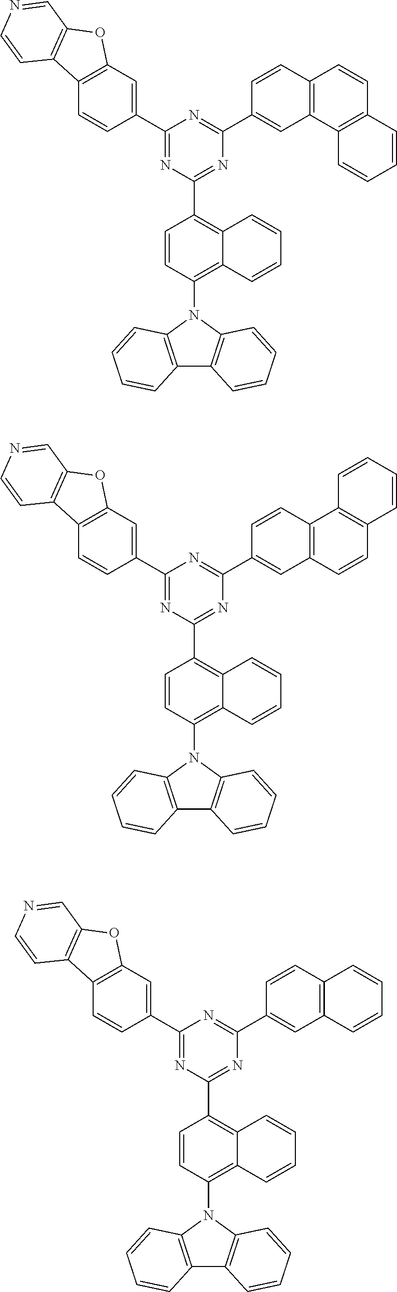 [SMILES: c1ccc2c(c1)ccc1ccc(-c3nc(-c4ccc5c(c4)oc4cnccc45)nc(-c4ccc(-n5c6ccccc6c6ccccc65)c5ccccc45)n3)cc12 ; c1ccc2c(c1)ccc1cc(-c3nc(-c4ccc5c(c4)oc4cnccc45)nc(-c4ccc(-n5c6ccccc6c6ccccc65)c5ccccc45)n3)ccc12 ; c1ccc2cc(-c3nc(-c4ccc5c(c4)oc4cnccc45)nc(-c4ccc(-n5c6ccccc6c6ccccc65)c5ccccc45)n3)ccc2c1]

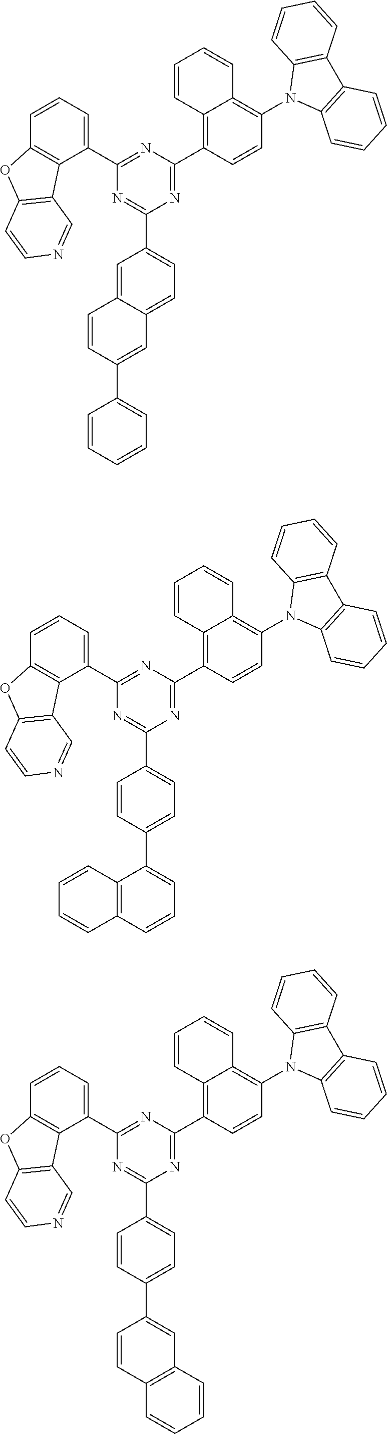 [SMILES: c1ccc(-c2ccc3cc(-c4nc(-c5ccc(-n6c7ccccc7c7ccccc76)c6ccccc56)nc(-c5cccc6oc7ccncc7c56)n4)ccc3c2)cc1 ; c1ccc2c(-c3ccc(-c4nc(-c5ccc(-n6c7ccccc7c7ccccc76)c6ccccc56)nc(-c5cccc6oc7ccncc7c56)n4)cc3)cccc2c1 ; c1ccc2cc(-c3ccc(-c4nc(-c5ccc(-n6c7ccccc7c7ccccc76)c6ccccc56)nc(-c5cccc6oc7ccncc7c56)n4)cc3)ccc2c1]

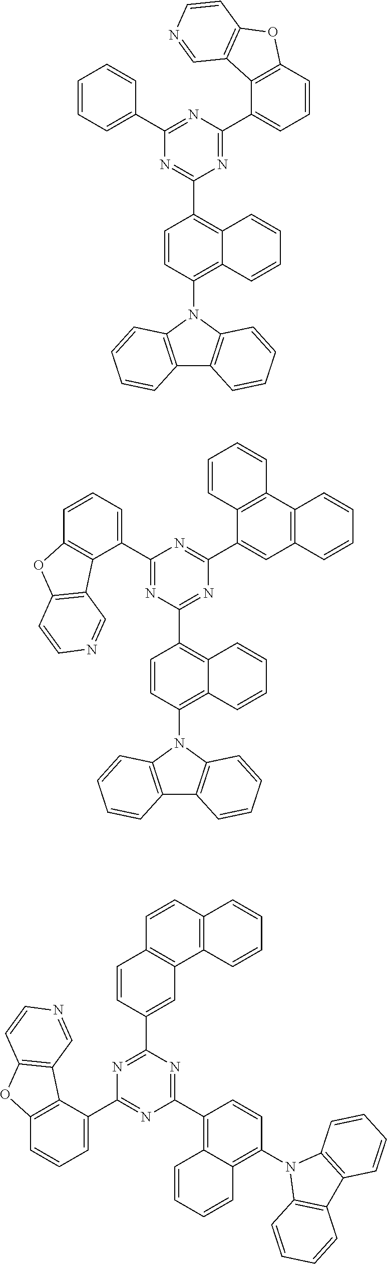 [SMILES: c1ccc(-c2nc(-c3ccc(-n4c5ccccc5c5ccccc54)c4ccccc34)nc(-c3cccc4oc5ccncc5c34)n2)cc1 ; c1ccc2c(c1)cc(-c1nc(-c3ccc(-n4c5ccccc5c5ccccc54)c4ccccc34)nc(-c3cccc4oc5ccncc5c34)n1)c1ccccc12 ; c1ccc2c(c1)ccc1ccc(-c3nc(-c4ccc(-n5c6ccccc6c6ccccc65)c5ccccc45)nc(-c4cccc5oc6ccncc6c45)n3)cc12]

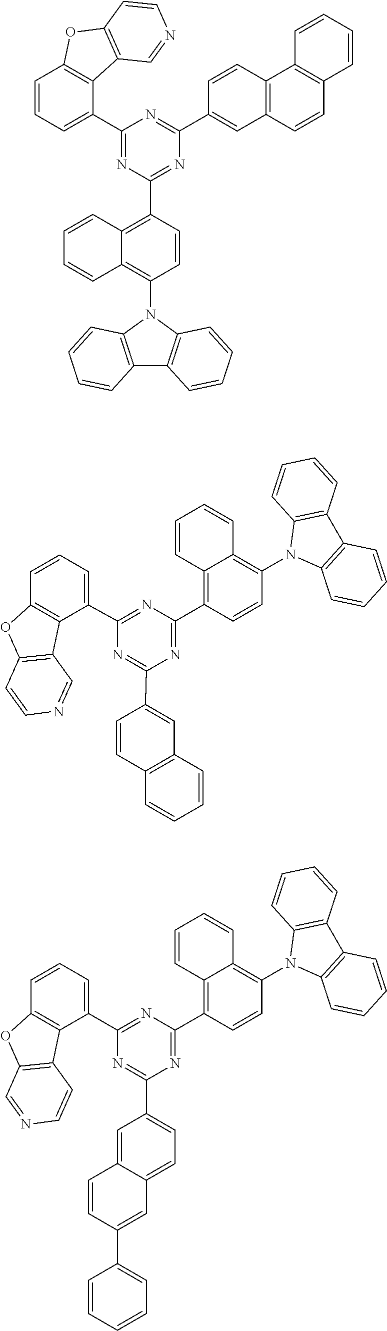 [SMILES: c1ccc2c(c1)ccc1cc(-c3nc(-c4ccc(-n5c6ccccc6c6ccccc65)c5ccccc45)nc(-c4cccc5oc6ccncc6c45)n3)ccc12 ; c1ccc2cc(-c3nc(-c4ccc(-n5c6ccccc6c6ccccc65)c5ccccc45)nc(-c4cccc5oc6ccncc6c45)n3)ccc2c1 ; c1ccc(-c2ccc3cc(-c4nc(-c5ccc(-n6c7ccccc7c7ccccc76)c6ccccc56)nc(-c5cccc6oc7cnccc7c56)n4)ccc3c2)cc1]

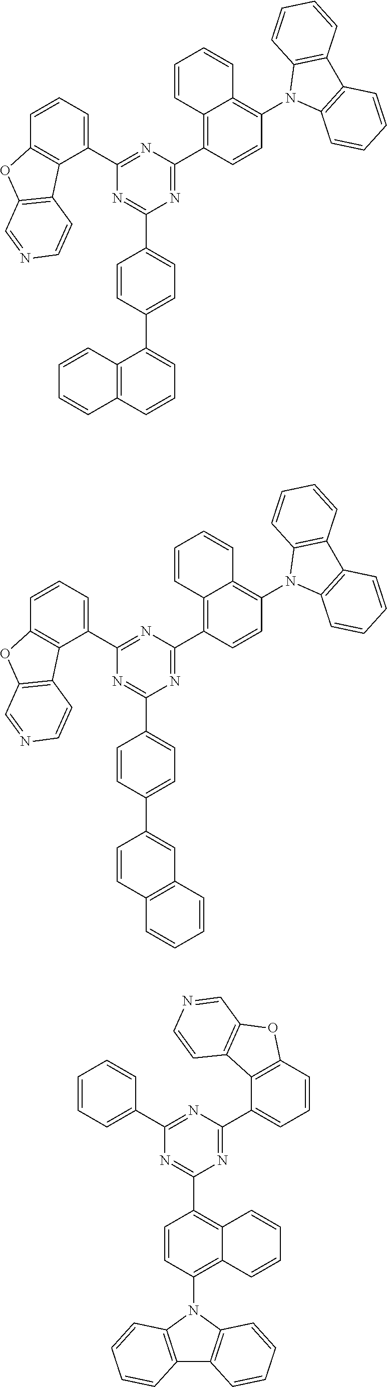 [SMILES: c1ccc2c(-c3ccc(-c4nc(-c5ccc(-n6c7ccccc7c7ccccc76)c6ccccc56)nc(-c5cccc6oc7cnccc7c56)n4)cc3)cccc2c1 ; c1ccc2cc(-c3ccc(-c4nc(-c5ccc(-n6c7ccccc7c7ccccc76)c6ccccc56)nc(-c5cccc6oc7cnccc7c56)n4)cc3)ccc2c1 ; c1ccc(-c2nc(-c3ccc(-n4c5ccccc5c5ccccc54)c4ccccc34)nc(-c3cccc4oc5cnccc5c34)n2)cc1]

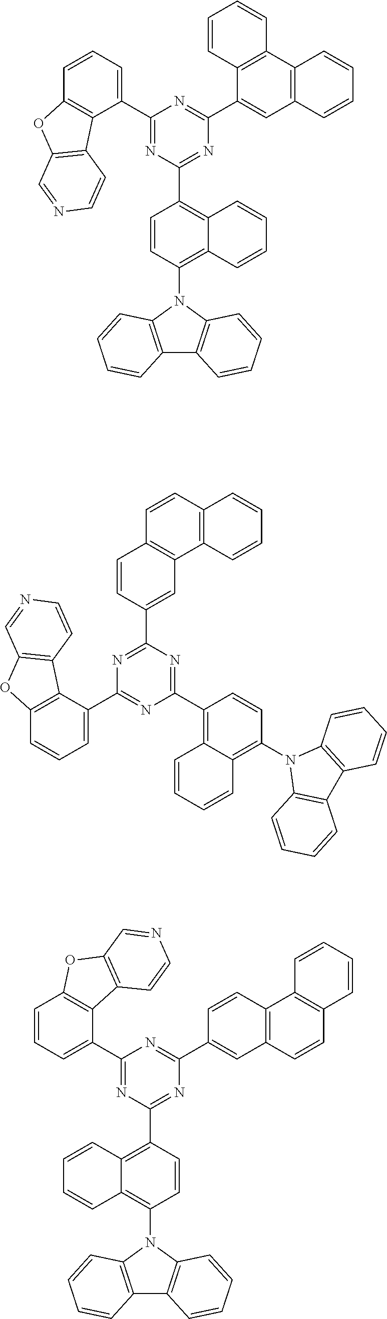 [SMILES: c1ccc2c(c1)cc(-c1nc(-c3ccc(-n4c5ccccc5c5ccccc54)c4ccccc34)nc(-c3cccc4oc5cnccc5c34)n1)c1ccccc12 ; c1ccc2c(c1)ccc1ccc(-c3nc(-c4ccc(-n5c6ccccc6c6ccccc65)c5ccccc45)nc(-c4cccc5oc6cnccc6c45)n3)cc12 ; c1ccc2c(c1)ccc1cc(-c3nc(-c4ccc(-n5c6ccccc6c6ccccc65)c5ccccc45)nc(-c4cccc5oc6cnccc6c45)n3)ccc12]

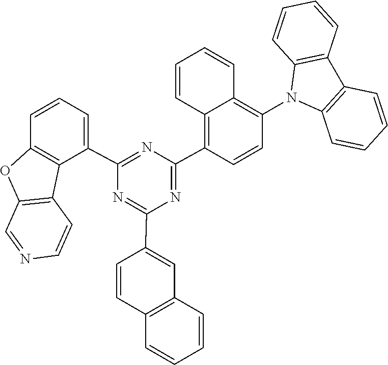 [SMILES: c1ccc2cc(-c3nc(-c4ccc(-n5c6ccccc6c6ccccc65)c5ccccc45)nc(-c4cccc5oc6cnccc6c45)n3)ccc2c1]

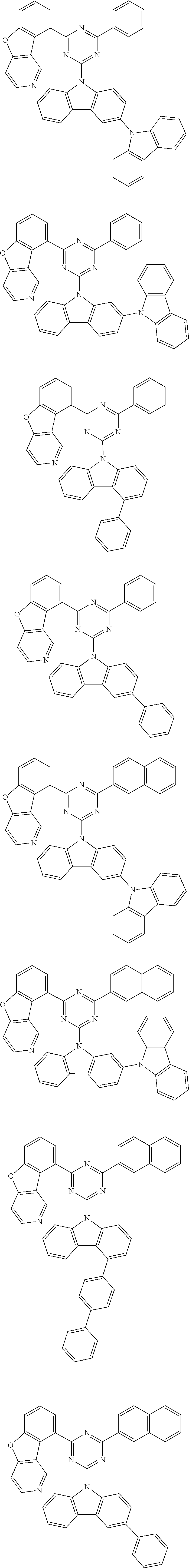

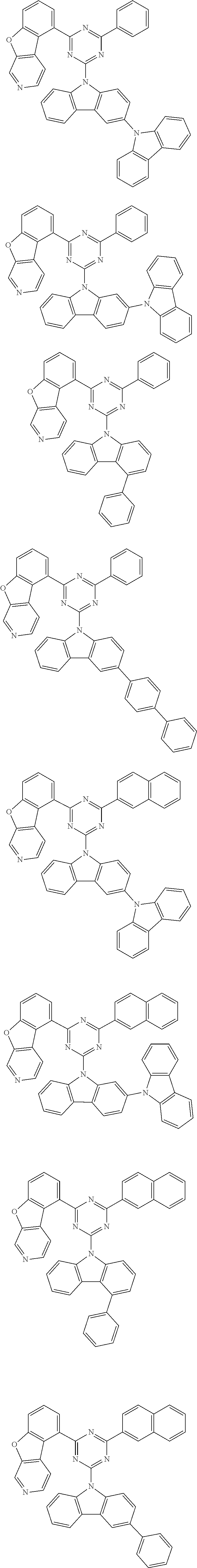

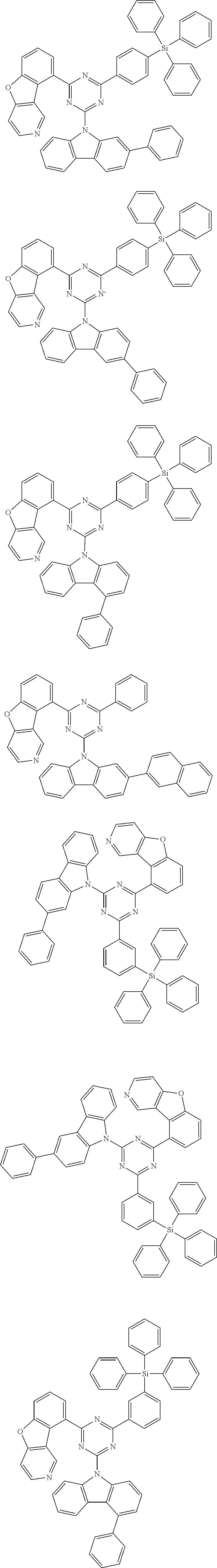

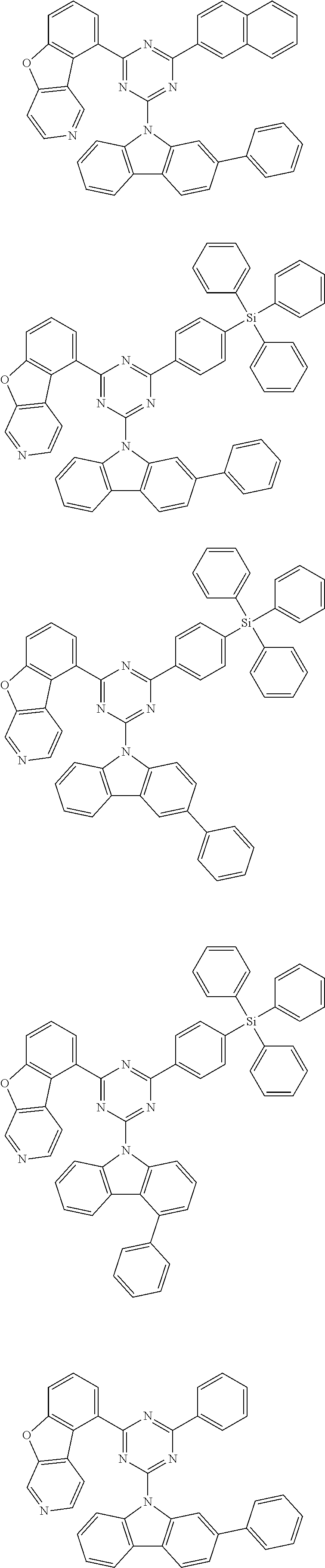 [SMILES: c1ccc(-c2ccc3c4ccccc4n(-c4nc(-c5ccc6ccccc6c5)nc(-c5cccc6oc7ccncc7c56)n4)c3c2)cc1 ; c1ccc(-c2ccc3c4ccccc4n(-c4nc(-c5ccc([Si](c6ccccc6)(c6ccccc6)c6ccccc6)cc5)nc(-c5cccc6oc7cnccc7c56)n4)c3c2)cc1 ; c1ccc(-c2ccc3c(c2)c2ccccc2n3-c2nc(-c3ccc([Si](c4ccccc4)(c4ccccc4)c4ccccc4)cc3)nc(-c3cccc4oc5cnccc5c34)n2)cc1 ; c1ccc(-c2cccc3c2c2ccccc2n3-c2nc(-c3ccc([Si](c4ccccc4)(c4ccccc4)c4ccccc4)cc3)nc(-c3cccc4oc5cnccc5c34)n2)cc1 ; c1ccc(-c2ccc3c4ccccc4n(-c4nc(-c5ccccc5)nc(-c5cccc6oc7cnccc7c56)n4)c3c2)cc1]

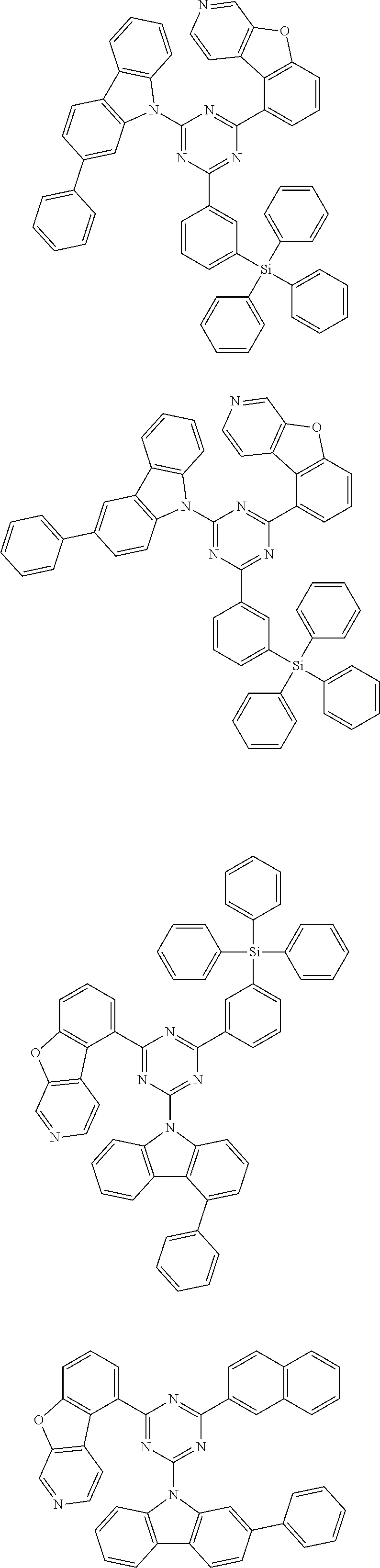 [SMILES: c1ccc(-c2ccc3c4ccccc4n(-c4nc(-c5cccc([Si](c6ccccc6)(c6ccccc6)c6ccccc6)c5)nc(-c5cccc6oc7cnccc7c56)n4)c3c2)cc1 ; c1ccc(-c2ccc3c(c2)c2ccccc2n3-c2nc(-c3cccc([Si](c4ccccc4)(c4ccccc4)c4ccccc4)c3)nc(-c3cccc4oc5cnccc5c34)n2)cc1 ; c1ccc(-c2cccc3c2c2ccccc2n3-c2nc(-c3cccc([Si](c4ccccc4)(c4ccccc4)c4ccccc4)c3)nc(-c3cccc4oc5cnccc5c34)n2)cc1 ; c1ccc(-c2ccc3c4ccccc4n(-c4nc(-c5ccc6ccccc6c5)nc(-c5cccc6oc7cnccc7c56)n4)c3c2)cc1]

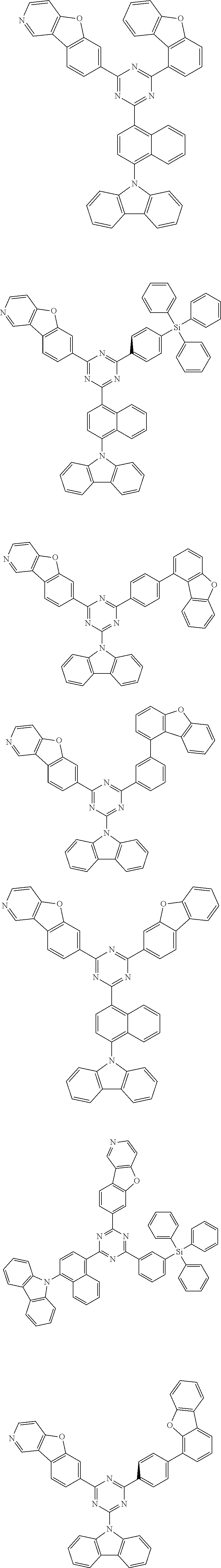

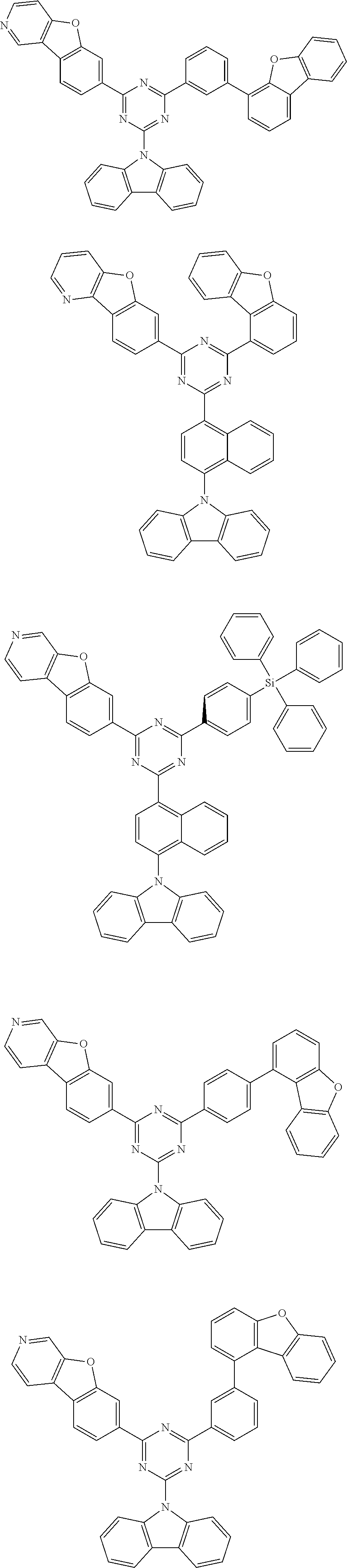

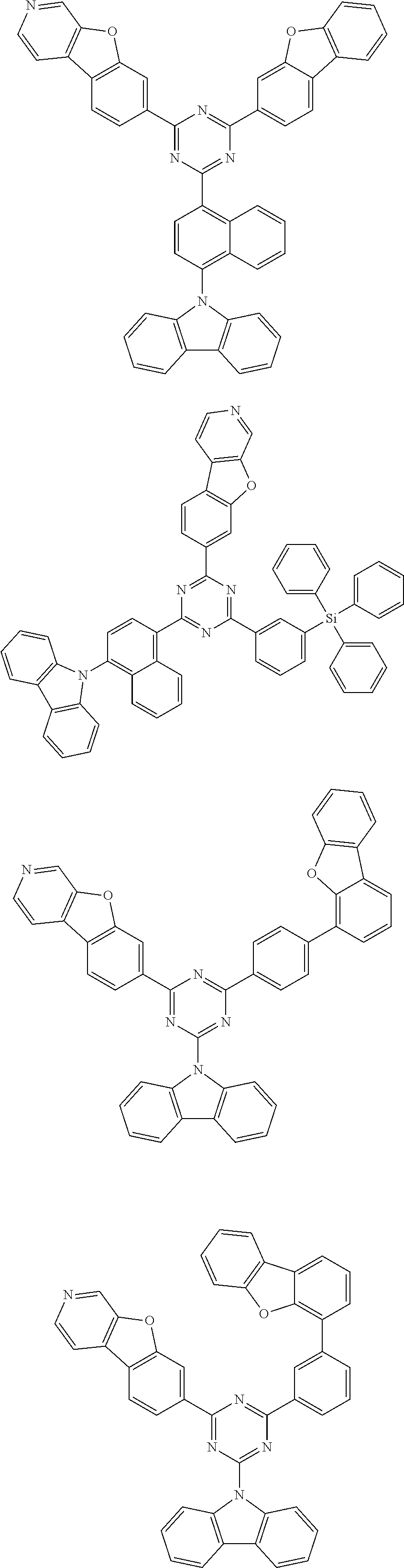

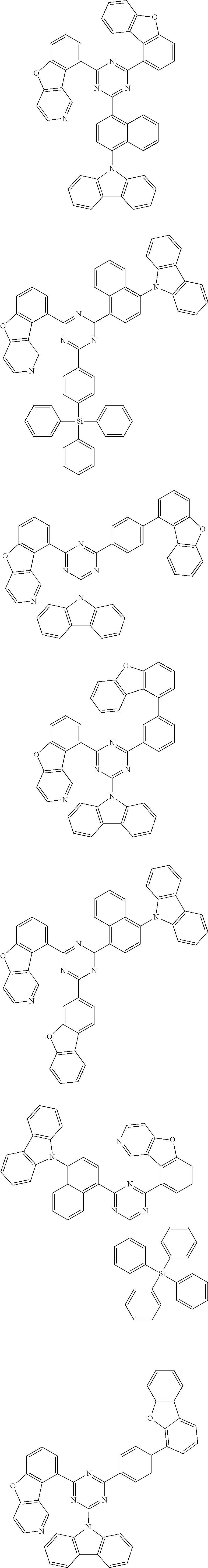

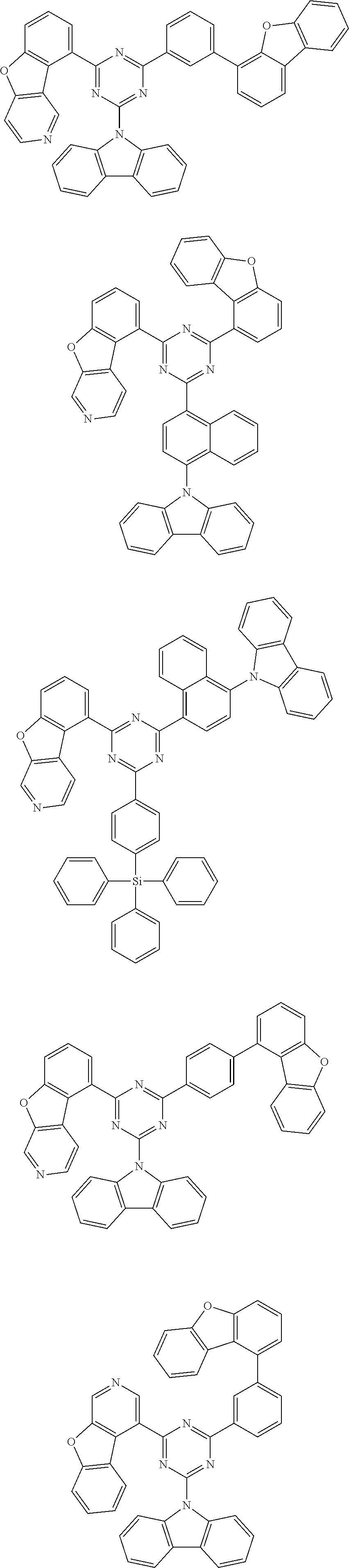 [SMILES: c1cc(-c2nc(-c3cccc4oc5ccncc5c34)nc(-n3c4ccccc4c4ccccc43)n2)cc(-c2cccc3c2oc2ccccc23)c1 ; c1ccc2c(c1)oc1cccc(-c3nc(-c4ccc(-n5c6ccccc6c6ccccc65)c5ccccc45)nc(-c4cccc5oc6cnccc6c45)n3)c12 ; c1ccc([Si](c2ccccc2)(c2ccccc2)c2ccc(-c3nc(-c4ccc(-n5c6ccccc6c6ccccc65)c5ccccc45)nc(-c4cccc5oc6cnccc6c45)n3)cc2)cc1 ; c1ccc2c(c1)oc1cccc(-c3ccc(-c4nc(-c5cccc6oc7cnccc7c56)nc(-n5c6ccccc6c6ccccc65)n4)cc3)c12 ; c1cc(-c2nc(-c3cncc4oc5ccccc5c34)nc(-n3c4ccccc4c4ccccc43)n2)cc(-c2cccc3oc4ccccc4c23)c1]

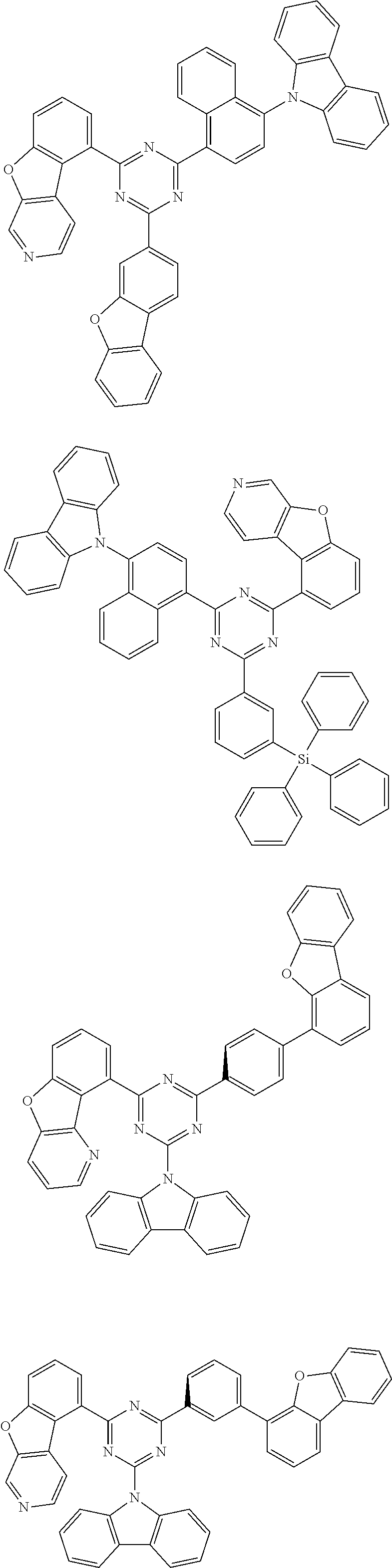

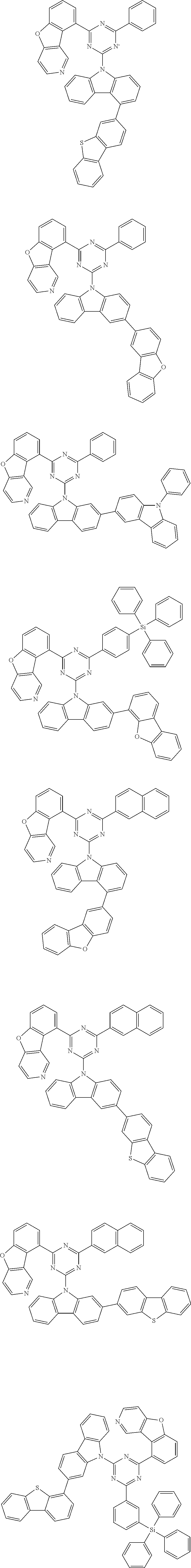

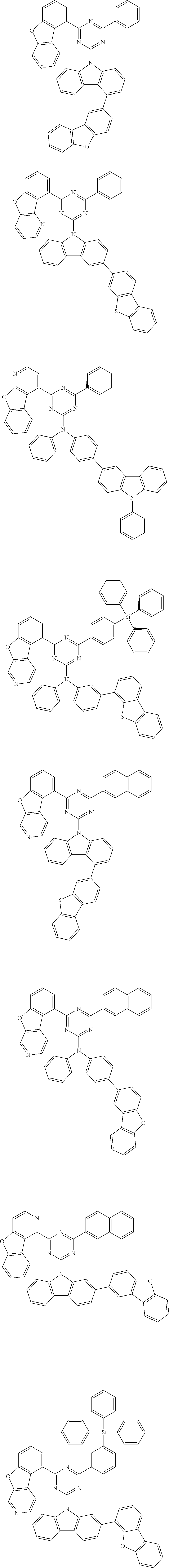

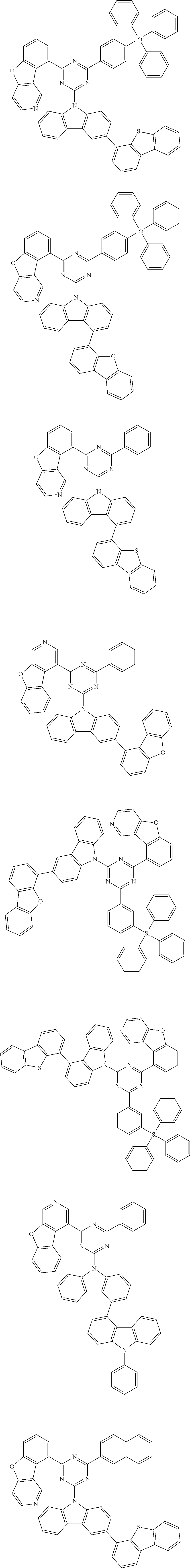

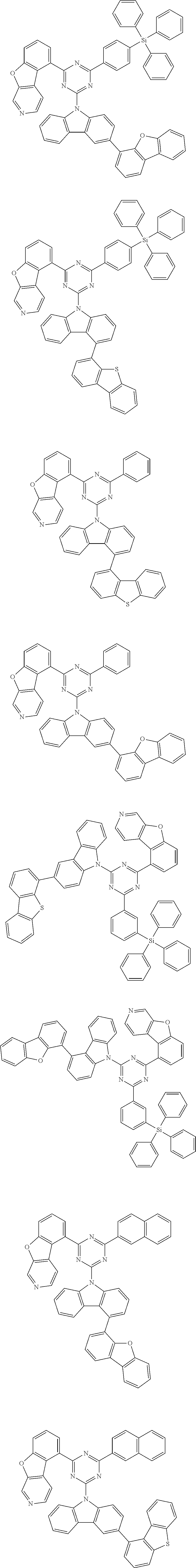

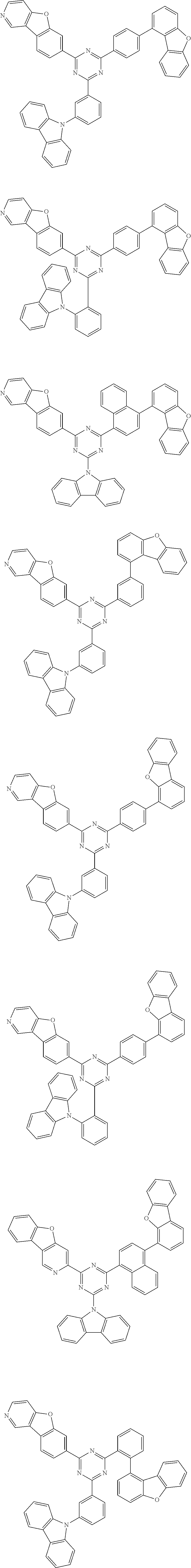

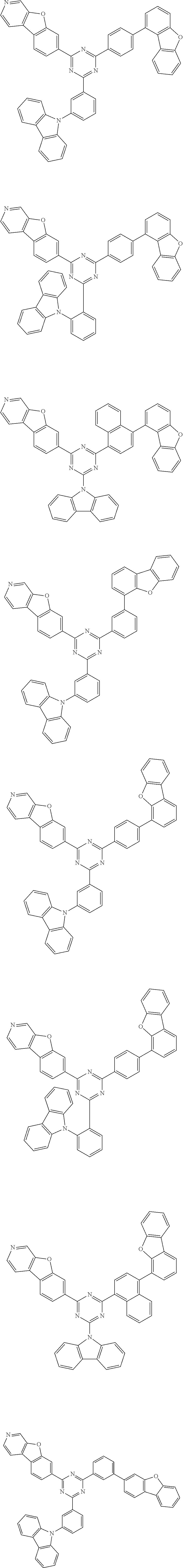

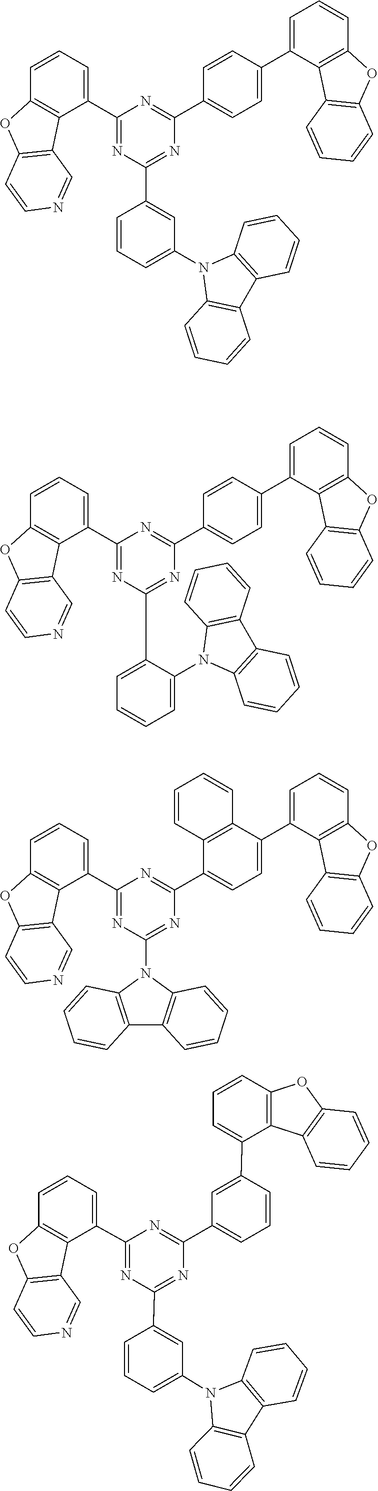

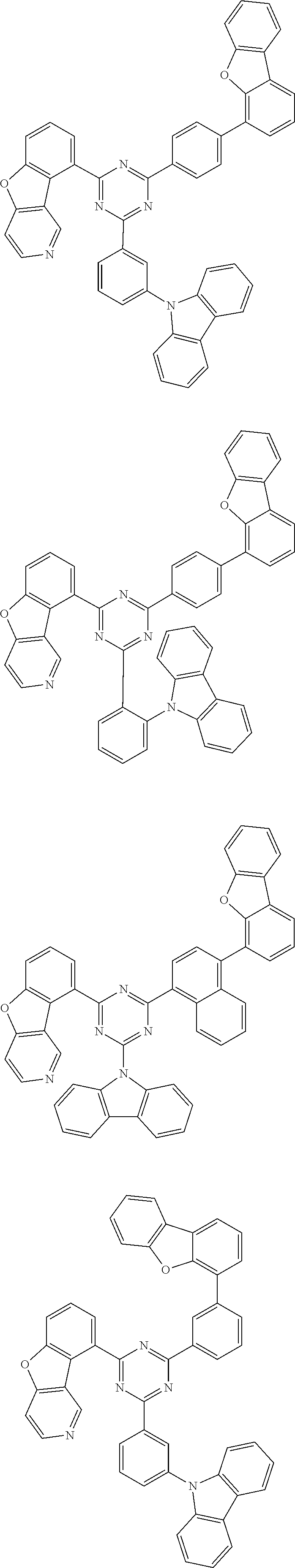 [SMILES: c1cc(-c2nc(-c3ccc(-c4cccc5c4oc4ccccc45)cc3)nc(-c3cccc4oc5ccncc5c34)n2)cc(-n2c3ccccc3c3ccccc32)c1 ; c1ccc(-n2c3ccccc3c3ccccc32)c(-c2nc(-c3ccc(-c4cccc5c4oc4ccccc45)cc3)nc(-c3cccc4oc5ccncc5c34)n2)c1 ; c1ccc2c(c1)oc1c(-c3ccc(-c4nc(-c5cccc6oc7ccncc7c56)nc(-n5c6ccccc6c6ccccc65)n4)c4ccccc34)cccc12 ; c1cc(-c2nc(-c3cccc(-n4c5ccccc5c5ccccc54)c3)nc(-c3cccc4oc5ccncc5c34)n2)cc(-c2cccc3c2oc2ccccc23)c1]

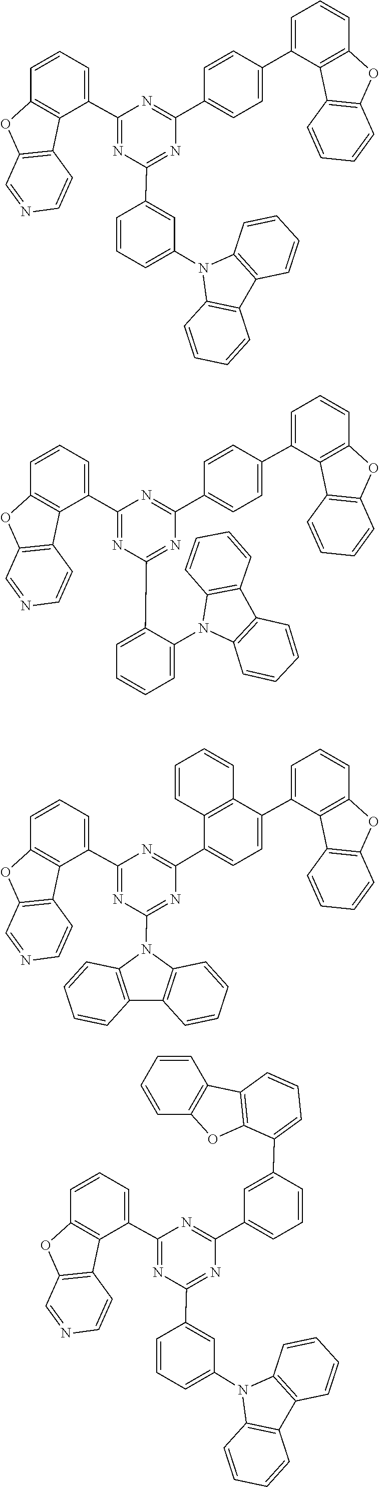

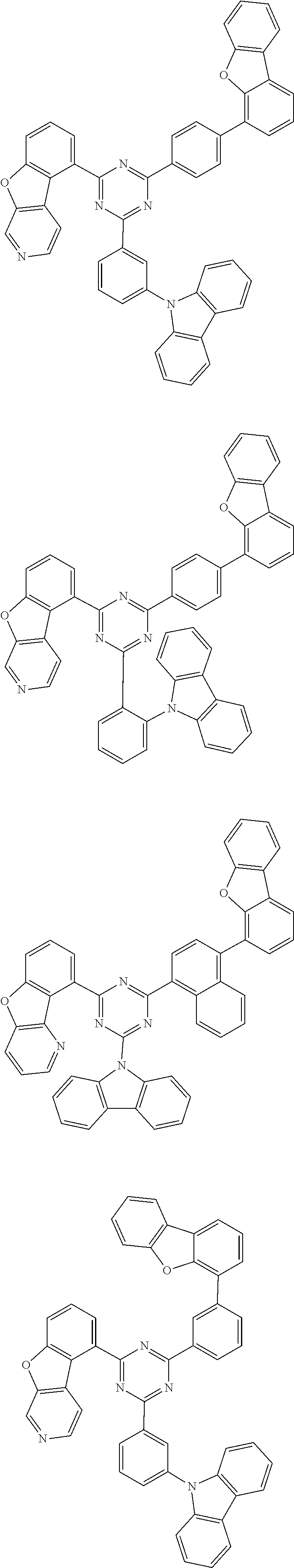 [SMILES: c1cc(-c2nc(-c3ccc(-c4cccc5c4oc4ccccc45)cc3)nc(-c3cccc4oc5cnccc5c34)n2)cc(-n2c3ccccc3c3ccccc32)c1 ; c1ccc(-n2c3ccccc3c3ccccc32)c(-c2nc(-c3ccc(-c4cccc5c4oc4ccccc45)cc3)nc(-c3cccc4oc5cnccc5c34)n2)c1 ; c1ccc2c(c1)oc1c(-c3ccc(-c4nc(-c5cccc6oc7cccnc7c56)nc(-n5c6ccccc6c6ccccc65)n4)c4ccccc34)cccc12 ; c1cc(-c2nc(-c3cccc(-n4c5ccccc5c5ccccc54)c3)nc(-c3cccc4oc5cnccc5c34)n2)cc(-c2cccc3c2oc2ccccc23)c1]

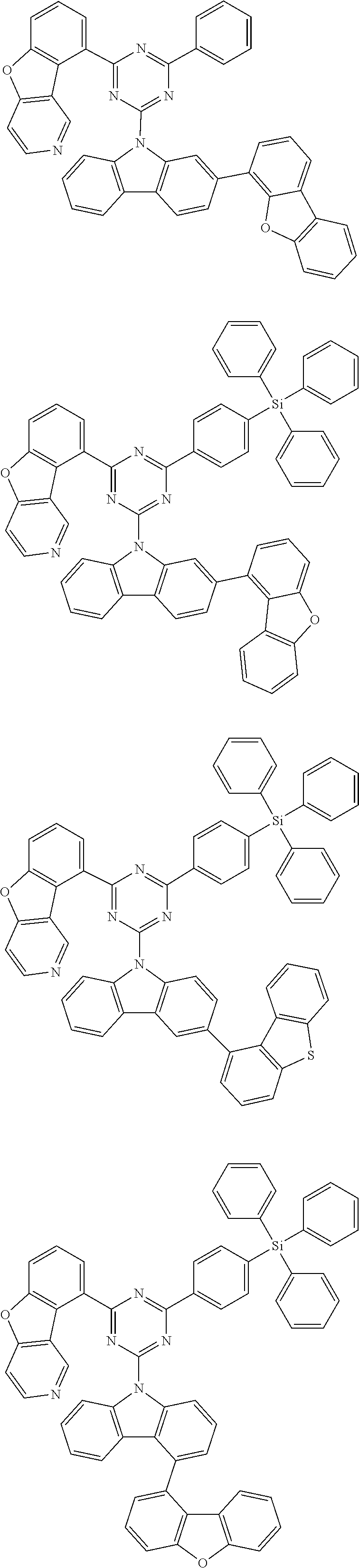 [SMILES: c1ccc(-c2nc(-c3cccc4oc5ccncc5c34)nc(-n3c4ccccc4c4ccc(-c5cccc6c5oc5ccccc56)cc43)n2)cc1 ; c1ccc([Si](c2ccccc2)(c2ccccc2)c2ccc(-c3nc(-c4cccc5oc6ccncc6c45)nc(-n4c5ccccc5c5ccc(-c6cccc7oc8ccccc8c67)cc54)n3)cc2)cc1 ; c1ccc([Si](c2ccccc2)(c2ccccc2)c2ccc(-c3nc(-c4cccc5oc6ccncc6c45)nc(-n4c5ccccc5c5cc(-c6cccc7sc8ccccc8c67)ccc54)n3)cc2)cc1 ; c1ccc([Si](c2ccccc2)(c2ccccc2)c2ccc(-c3nc(-c4cccc5oc6ccncc6c45)nc(-n4c5ccccc5c5c(-c6cccc7oc8ccccc8c67)cccc54)n3)cc2)cc1]

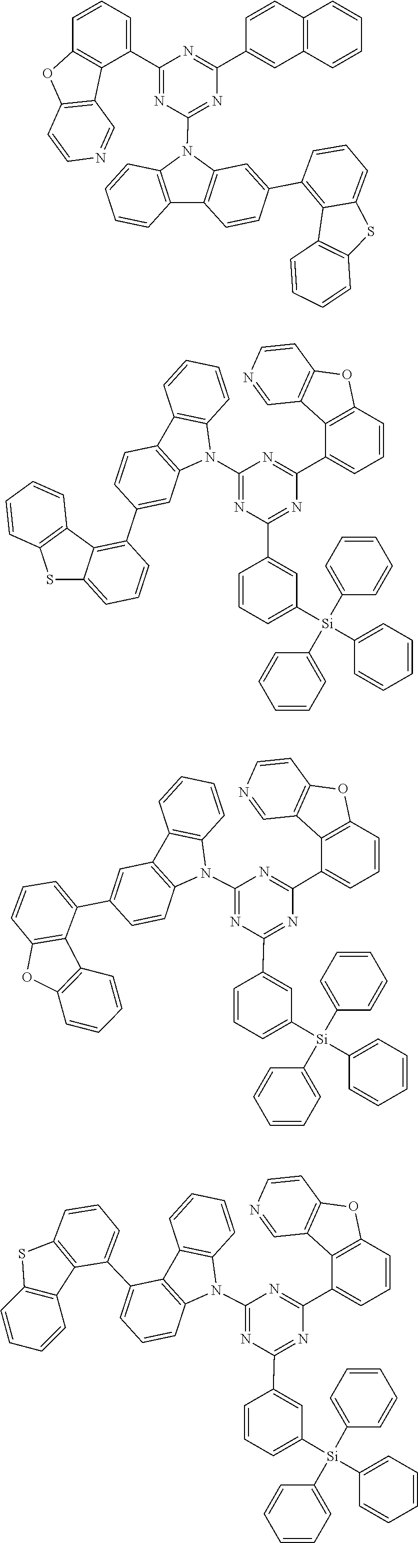

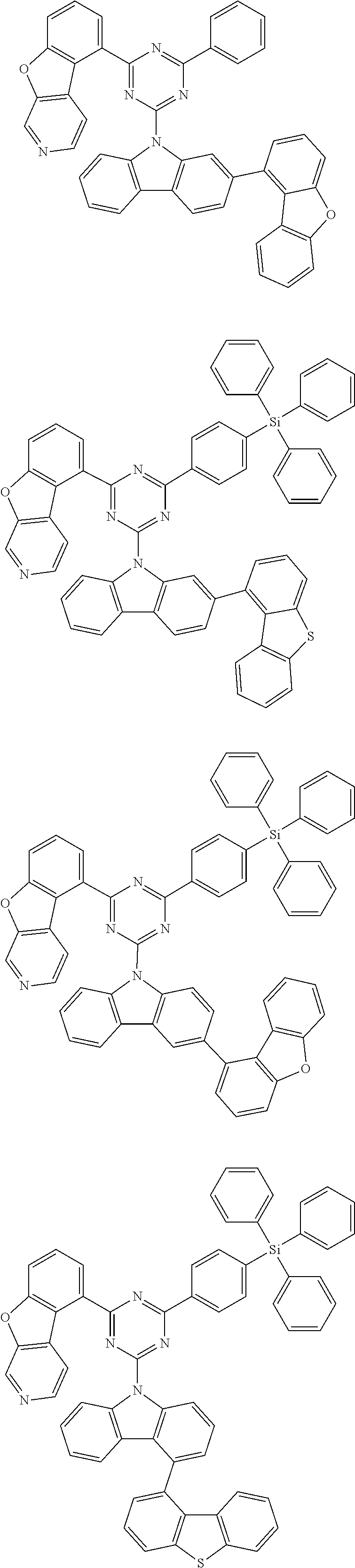 [SMILES: c1ccc(-c2nc(-c3cccc4oc5cnccc5c34)nc(-n3c4ccccc4c4ccc(-c5cccc6oc7ccccc7c56)cc43)n2)cc1 ; c1ccc([Si](c2ccccc2)(c2ccccc2)c2ccc(-c3nc(-c4cccc5oc6cnccc6c45)nc(-n4c5ccccc5c5ccc(-c6cccc7sc8ccccc8c67)cc54)n3)cc2)cc1 ; c1ccc([Si](c2ccccc2)(c2ccccc2)c2ccc(-c3nc(-c4cccc5oc6cnccc6c45)nc(-n4c5ccccc5c5cc(-c6cccc7oc8ccccc8c67)ccc54)n3)cc2)cc1 ; c1ccc([Si](c2ccccc2)(c2ccccc2)c2ccc(-c3nc(-c4cccc5oc6cnccc6c45)nc(-n4c5ccccc5c5c(-c6cccc7sc8ccccc8c67)cccc54)n3)cc2)cc1]

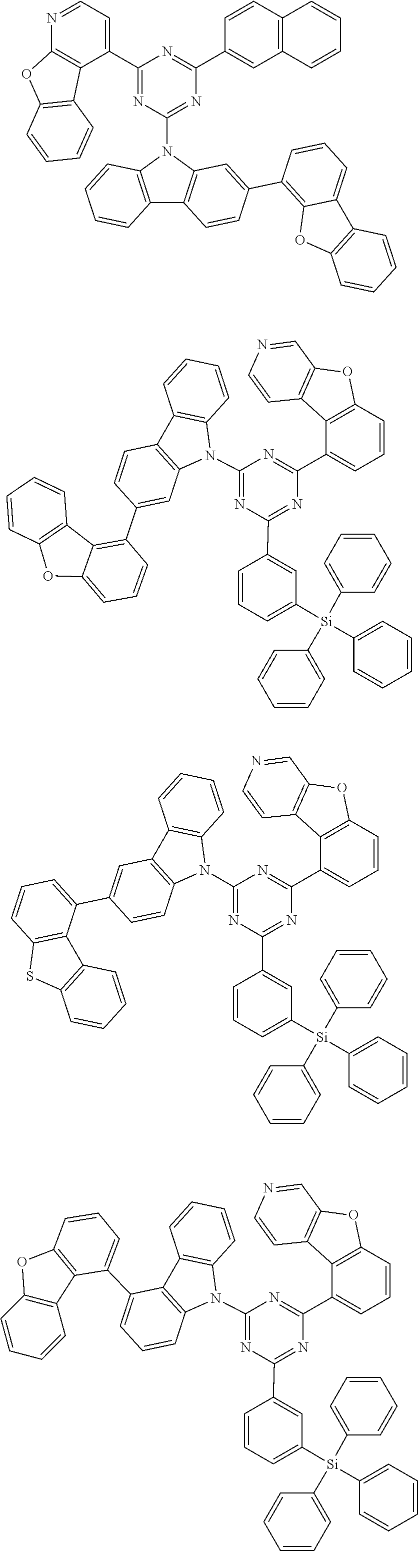 [SMILES: c1ccc2cc(-c3nc(-c4ccnc5oc6ccccc6c45)nc(-n4c5ccccc5c5ccc(-c6cccc7c6oc6ccccc67)cc54)n3)ccc2c1 ; c1ccc([Si](c2ccccc2)(c2ccccc2)c2cccc(-c3nc(-c4cccc5oc6cnccc6c45)nc(-n4c5ccccc5c5ccc(-c6cccc7oc8ccccc8c67)cc54)n3)c2)cc1 ; c1ccc([Si](c2ccccc2)(c2ccccc2)c2cccc(-c3nc(-c4cccc5oc6cnccc6c45)nc(-n4c5ccccc5c5cc(-c6cccc7sc8ccccc8c67)ccc54)n3)c2)cc1 ; c1ccc([Si](c2ccccc2)(c2ccccc2)c2cccc(-c3nc(-c4cccc5oc6cnccc6c45)nc(-n4c5ccccc5c5c(-c6cccc7oc8ccccc8c67)cccc54)n3)c2)cc1]

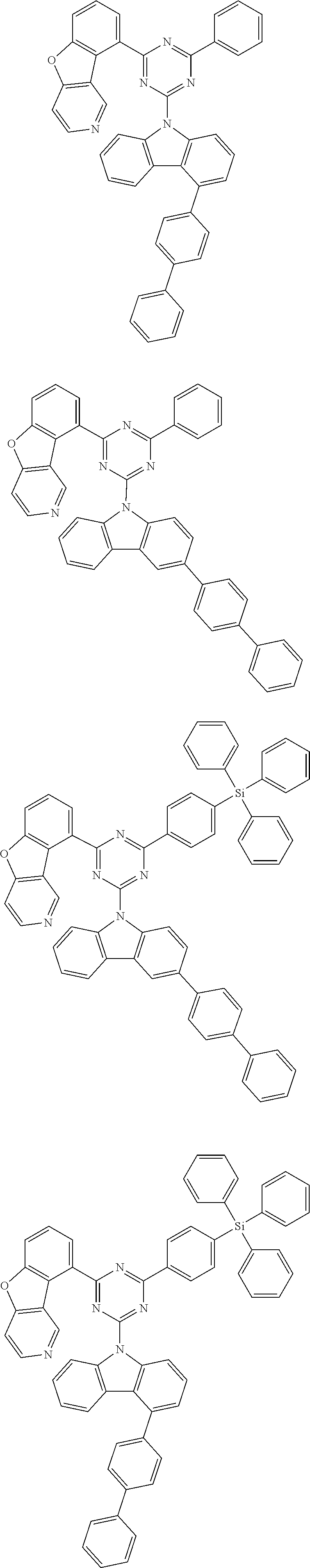 [SMILES: c1ccc(-c2ccc(-c3cccc4c3c3ccccc3n4-c3nc(-c4ccccc4)nc(-c4cccc5oc6ccncc6c45)n3)cc2)cc1 ; c1ccc(-c2ccc(-c3ccc4c(c3)c3ccccc3n4-c3nc(-c4ccccc4)nc(-c4cccc5oc6ccncc6c45)n3)cc2)cc1 ; c1ccc(-c2ccc(-c3ccc4c(c3)c3ccccc3n4-c3nc(-c4ccc([Si](c5ccccc5)(c5ccccc5)c5ccccc5)cc4)nc(-c4cccc5oc6ccncc6c45)n3)cc2)cc1 ; c1ccc(-c2ccc(-c3cccc4c3c3ccccc3n4-c3nc(-c4ccc([Si](c5ccccc5)(c5ccccc5)c5ccccc5)cc4)nc(-c4cccc5oc6ccncc6c45)n3)cc2)cc1]

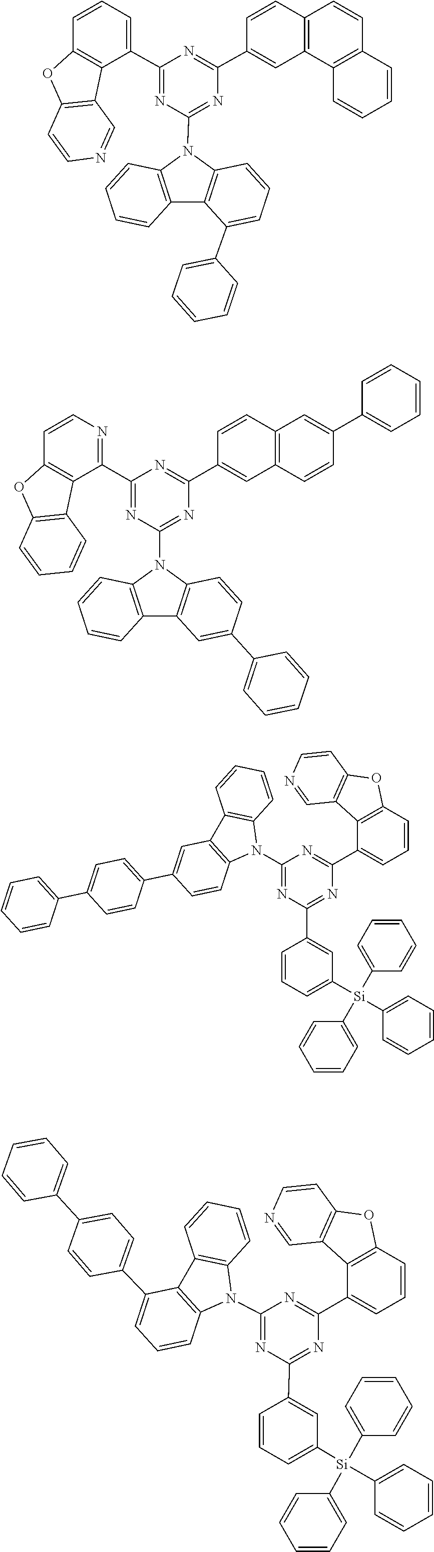

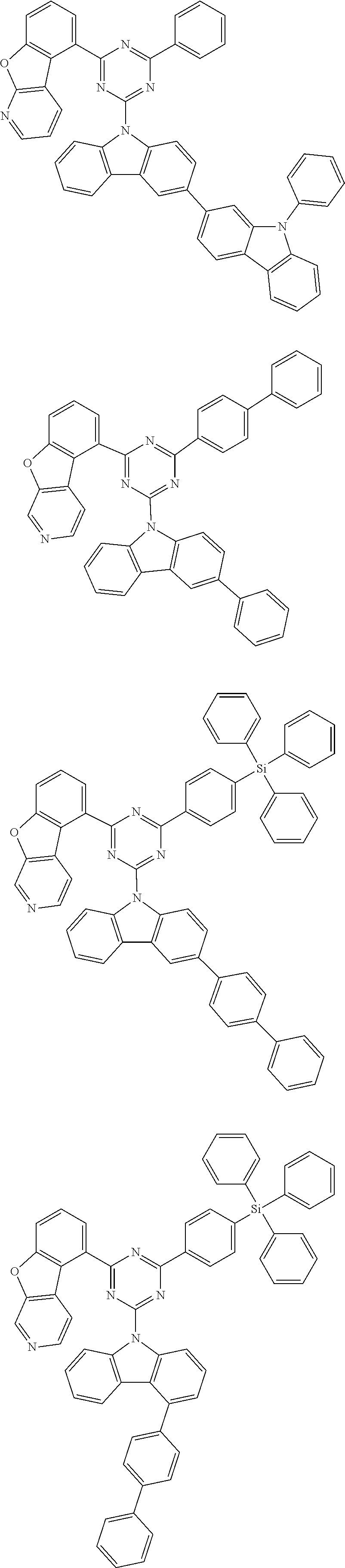

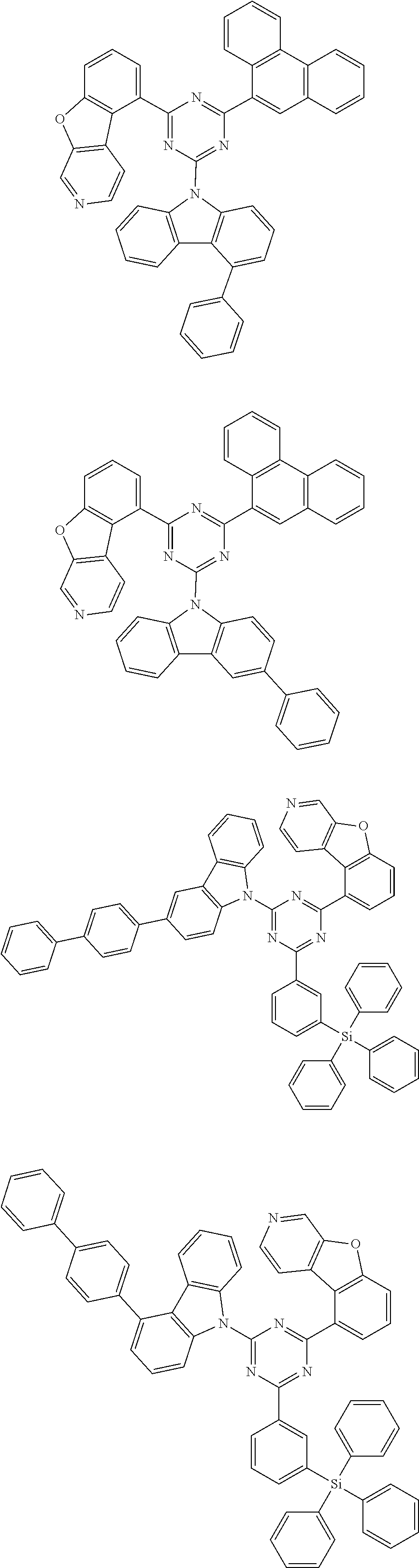 [SMILES: c1ccc(-c2cccc3c2c2ccccc2n3-c2nc(-c3cc4ccccc4c4ccccc34)nc(-c3cccc4oc5cnccc5c34)n2)cc1 ; c1ccc(-c2ccc3c(c2)c2ccccc2n3-c2nc(-c3cc4ccccc4c4ccccc34)nc(-c3cccc4oc5cnccc5c34)n2)cc1 ; c1ccc(-c2ccc(-c3ccc4c(c3)c3ccccc3n4-c3nc(-c4cccc([Si](c5ccccc5)(c5ccccc5)c5ccccc5)c4)nc(-c4cccc5oc6cnccc6c45)n3)cc2)cc1 ; c1ccc(-c2ccc(-c3cccc4c3c3ccccc3n4-c3nc(-c4cccc([Si](c5ccccc5)(c5ccccc5)c5ccccc5)c4)nc(-c4cccc5oc6cnccc6c45)n3)cc2)cc1]

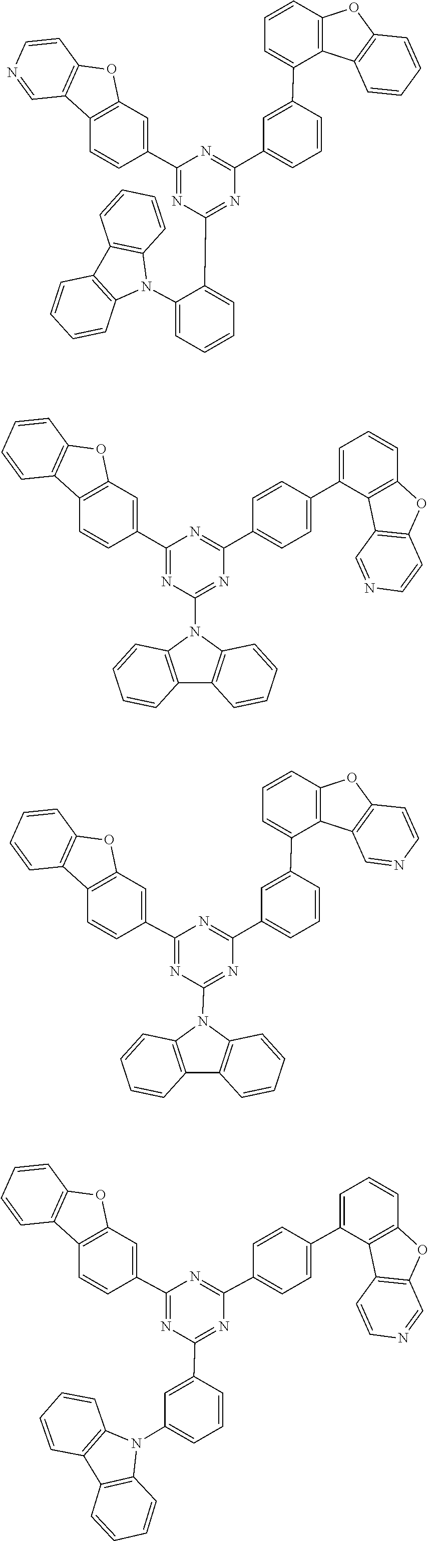 [SMILES: c1cc(-c2nc(-c3ccc4c(c3)oc3ccncc34)nc(-c3ccccc3-n3c4ccccc4c4ccccc43)n2)cc(-c2cccc3oc4ccccc4c23)c1 ; c1ccc2c(c1)oc1cc(-c3nc(-c4ccc(-c5cccc6oc7ccncc7c56)cc4)nc(-n4c5ccccc5c5ccccc54)n3)ccc12 ; c1cc(-c2nc(-c3ccc4c(c3)oc3ccccc34)nc(-n3c4ccccc4c4ccccc43)n2)cc(-c2cccc3oc4ccncc4c23)c1 ; c1cc(-c2nc(-c3ccc(-c4cccc5oc6cnccc6c45)cc3)nc(-c3ccc4c(c3)oc3ccccc34)n2)cc(-n2c3ccccc3c3ccccc32)c1]

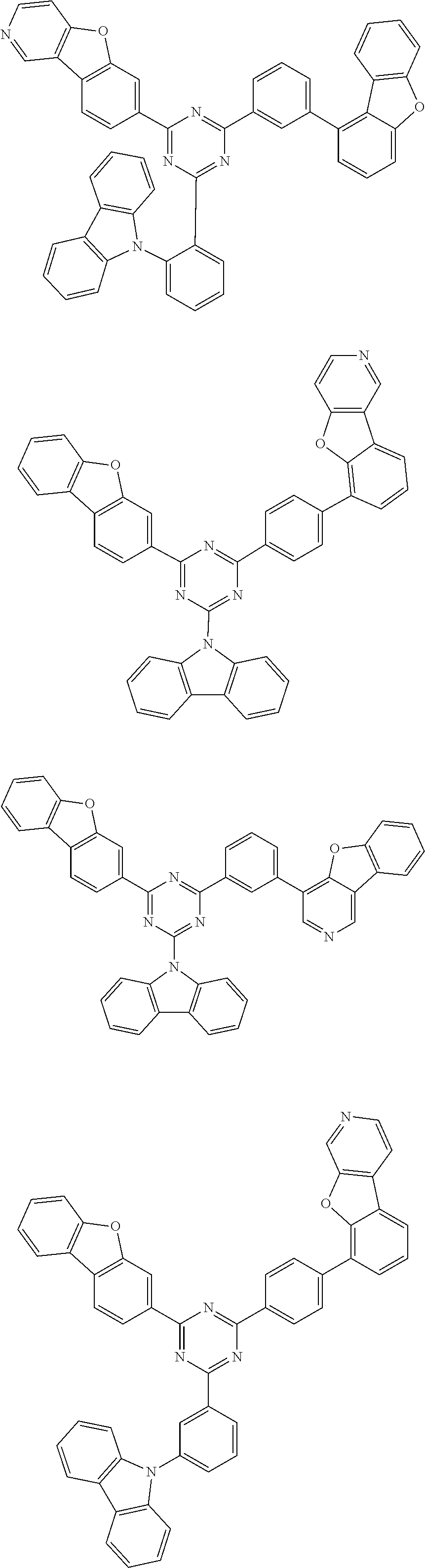 [SMILES: c1cc(-c2nc(-c3ccc4c(c3)oc3ccncc34)nc(-c3ccccc3-n3c4ccccc4c4ccccc43)n2)cc(-c2cccc3oc4ccccc4c23)c1 ; c1ccc2c(c1)oc1cc(-c3nc(-c4ccc(-c5cccc6c5oc5ccncc56)cc4)nc(-n4c5ccccc5c5ccccc54)n3)ccc12 ; c1cc(-c2nc(-c3ccc4c(c3)oc3ccccc34)nc(-n3c4ccccc4c4ccccc43)n2)cc(-c2cncc3c2oc2ccccc23)c1 ; c1cc(-c2nc(-c3ccc(-c4cccc5c4oc4cnccc45)cc3)nc(-c3ccc4c(c3)oc3ccccc34)n2)cc(-n2c3ccccc3c3ccccc32)c1]

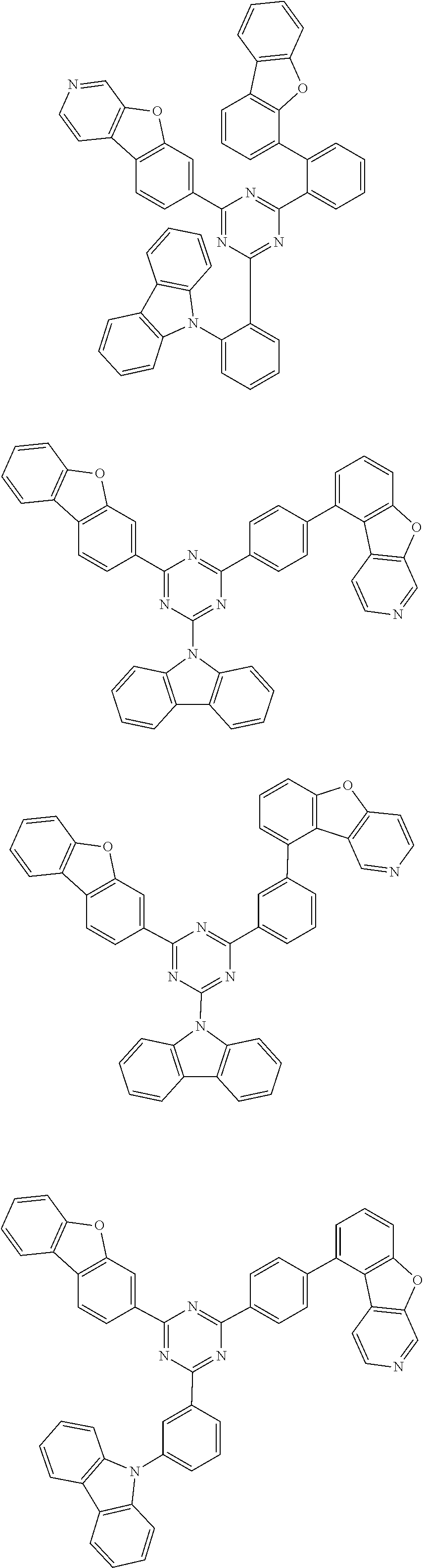 [SMILES: c1ccc(-c2cccc3c2oc2ccccc23)c(-c2nc(-c3ccc4c(c3)oc3cnccc34)nc(-c3ccccc3-n3c4ccccc4c4ccccc43)n2)c1 ; c1ccc2c(c1)oc1cc(-c3nc(-c4ccc(-c5cccc6oc7cnccc7c56)cc4)nc(-n4c5ccccc5c5ccccc54)n3)ccc12 ; c1cc(-c2nc(-c3ccc4c(c3)oc3ccccc34)nc(-n3c4ccccc4c4ccccc43)n2)cc(-c2cccc3oc4ccncc4c23)c1 ; c1cc(-c2nc(-c3ccc(-c4cccc5oc6cnccc6c45)cc3)nc(-c3ccc4c(c3)oc3ccccc34)n2)cc(-n2c3ccccc3c3ccccc32)c1]

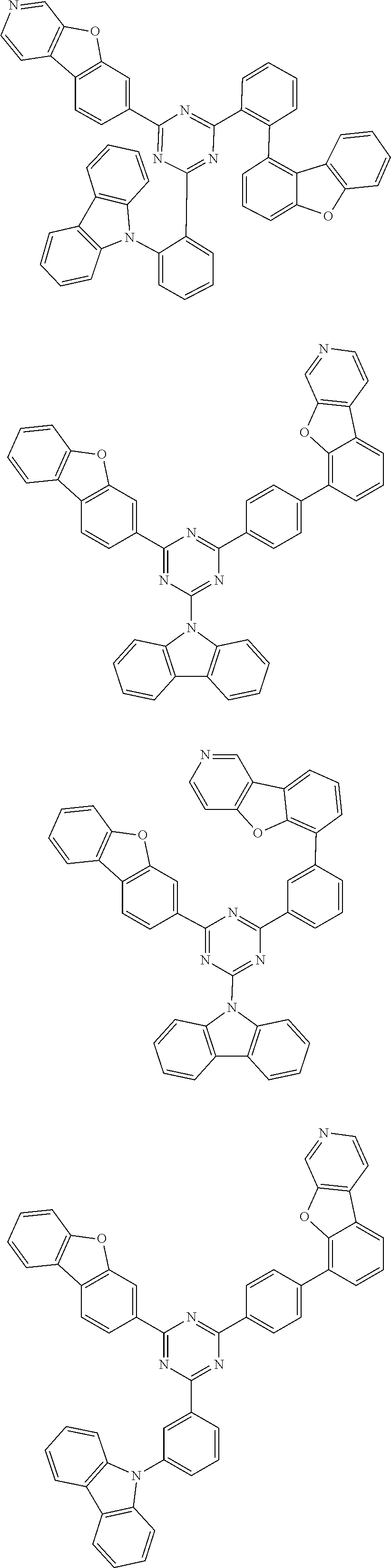 [SMILES: c1ccc(-c2cccc3oc4ccccc4c23)c(-c2nc(-c3ccc4c(c3)oc3cnccc34)nc(-c3ccccc3-n3c4ccccc4c4ccccc43)n2)c1 ; c1ccc2c(c1)oc1cc(-c3nc(-c4ccc(-c5cccc6c5oc5cnccc56)cc4)nc(-n4c5ccccc5c5ccccc54)n3)ccc12 ; c1cc(-c2nc(-c3ccc4c(c3)oc3ccccc34)nc(-n3c4ccccc4c4ccccc43)n2)cc(-c2cccc3c2oc2ccncc23)c1 ; c1cc(-c2nc(-c3ccc(-c4cccc5c4oc4cnccc45)cc3)nc(-c3ccc4c(c3)oc3ccccc34)n2)cc(-n2c3ccccc3c3ccccc32)c1]

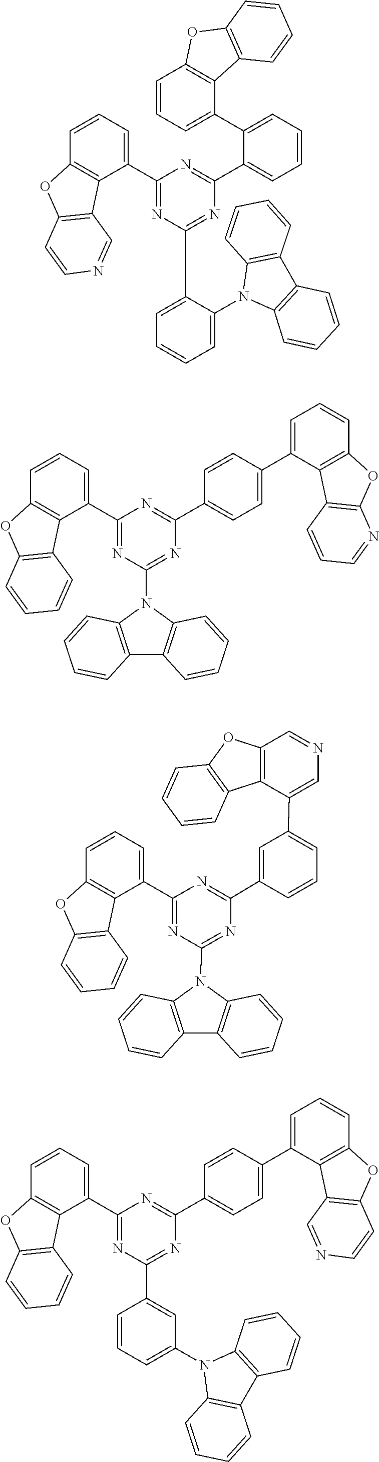 [SMILES: c1ccc(-c2cccc3oc4ccccc4c23)c(-c2nc(-c3ccccc3-n3c4ccccc4c4ccccc43)nc(-c3cccc4oc5ccncc5c34)n2)c1 ; c1ccc2c(c1)oc1cccc(-c3nc(-c4ccc(-c5cccc6oc7ncccc7c56)cc4)nc(-n4c5ccccc5c5ccccc54)n3)c12 ; c1cc(-c2nc(-c3cccc4oc5ccccc5c34)nc(-n3c4ccccc4c4ccccc43)n2)cc(-c2cncc3oc4ccccc4c23)c1 ; c1cc(-c2nc(-c3ccc(-c4cccc5oc6ccncc6c45)cc3)nc(-c3cccc4oc5ccccc5c34)n2)cc(-n2c3ccccc3c3ccccc32)c1]

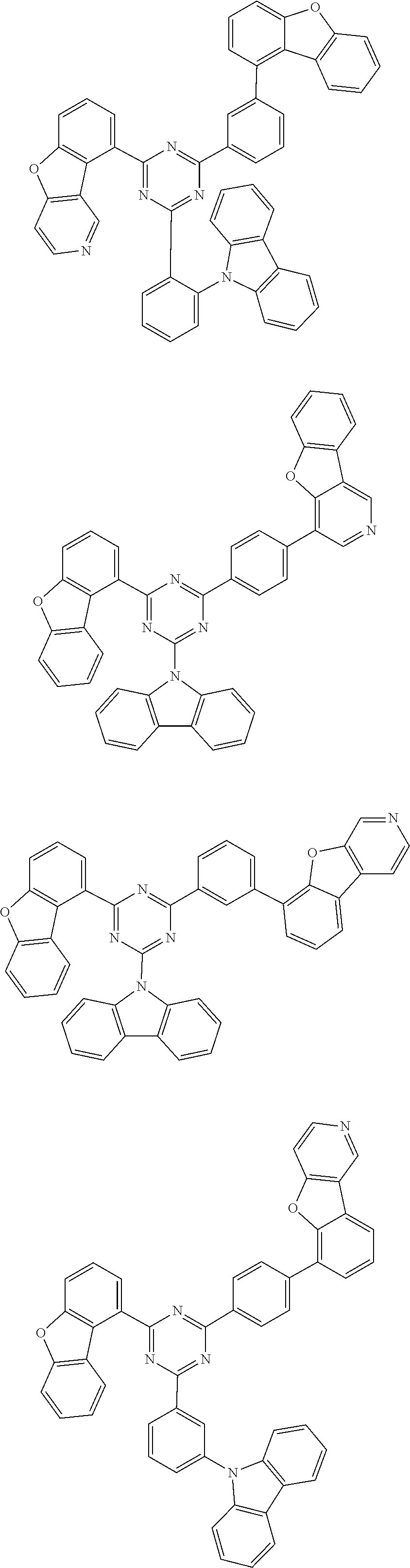 [SMILES: c1cc(-c2nc(-c3ccccc3-n3c4ccccc4c4ccccc43)nc(-c3cccc4oc5ccncc5c34)n2)cc(-c2cccc3oc4ccccc4c23)c1 ; c1ccc2c(c1)oc1c(-c3ccc(-c4nc(-c5cccc6oc7ccccc7c56)nc(-n5c6ccccc6c6ccccc65)n4)cc3)cncc12 ; c1cc(-c2nc(-c3cccc4oc5ccccc5c34)nc(-n3c4ccccc4c4ccccc43)n2)cc(-c2cccc3c2oc2cnccc23)c1 ; c1cc(-c2nc(-c3ccc(-c4cccc5c4oc4ccncc45)cc3)nc(-c3cccc4oc5ccccc5c34)n2)cc(-n2c3ccccc3c3ccccc32)c1]

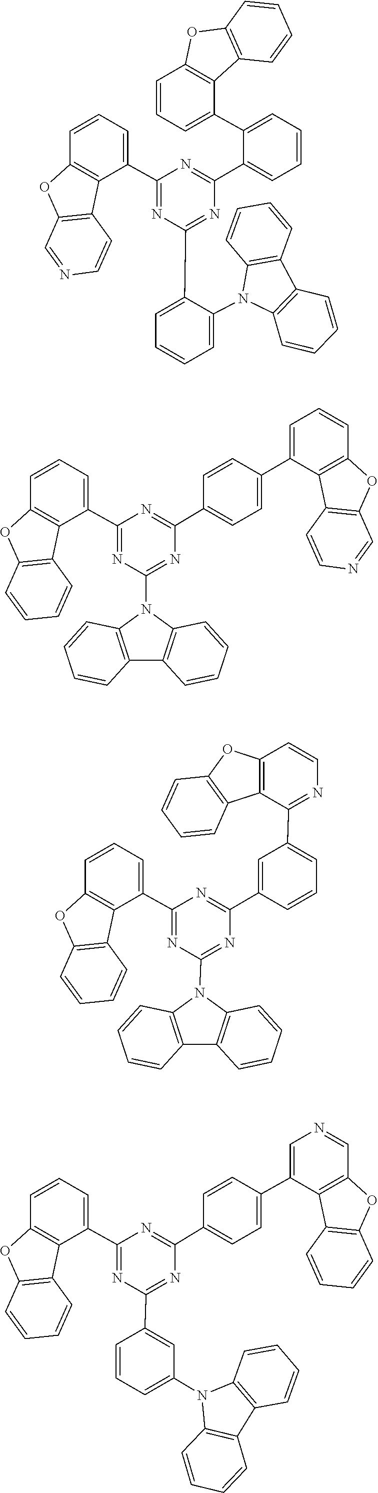 [SMILES: c1ccc(-c2cccc3oc4ccccc4c23)c(-c2nc(-c3ccccc3-n3c4ccccc4c4ccccc43)nc(-c3cccc4oc5cnccc5c34)n2)c1 ; c1ccc2c(c1)oc1cccc(-c3nc(-c4ccc(-c5cccc6oc7cnccc7c56)cc4)nc(-n4c5ccccc5c5ccccc54)n3)c12 ; c1cc(-c2nc(-c3cccc4oc5ccccc5c34)nc(-n3c4ccccc4c4ccccc43)n2)cc(-c2nccc3oc4ccccc4c23)c1 ; c1cc(-c2nc(-c3ccc(-c4cncc5oc6ccccc6c45)cc3)nc(-c3cccc4oc5ccccc5c34)n2)cc(-n2c3ccccc3c3ccccc32)c1]

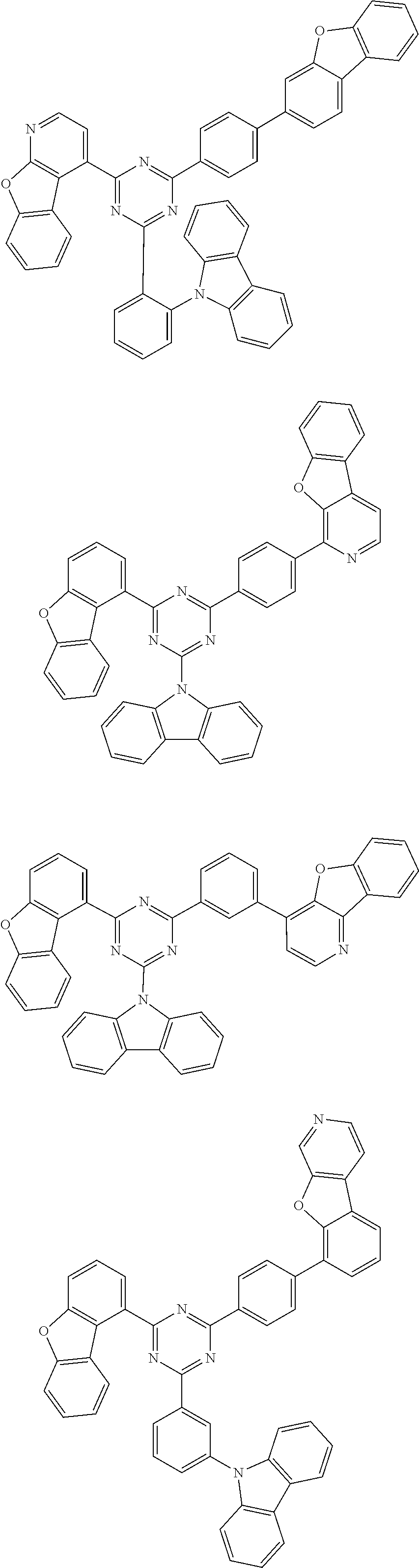 [SMILES: c1ccc(-n2c3ccccc3c3ccccc32)c(-c2nc(-c3ccc(-c4ccc5c(c4)oc4ccccc45)cc3)nc(-c3ccnc4oc5ccccc5c34)n2)c1 ; c1ccc2c(c1)oc1c(-c3ccc(-c4nc(-c5cccc6oc7ccccc7c56)nc(-n5c6ccccc6c6ccccc65)n4)cc3)nccc12 ; c1cc(-c2nc(-c3cccc4oc5ccccc5c34)nc(-n3c4ccccc4c4ccccc43)n2)cc(-c2ccnc3c2oc2ccccc23)c1 ; c1cc(-c2nc(-c3ccc(-c4cccc5c4oc4cnccc45)cc3)nc(-c3cccc4oc5ccccc5c34)n2)cc(-n2c3ccccc3c3ccccc32)c1]

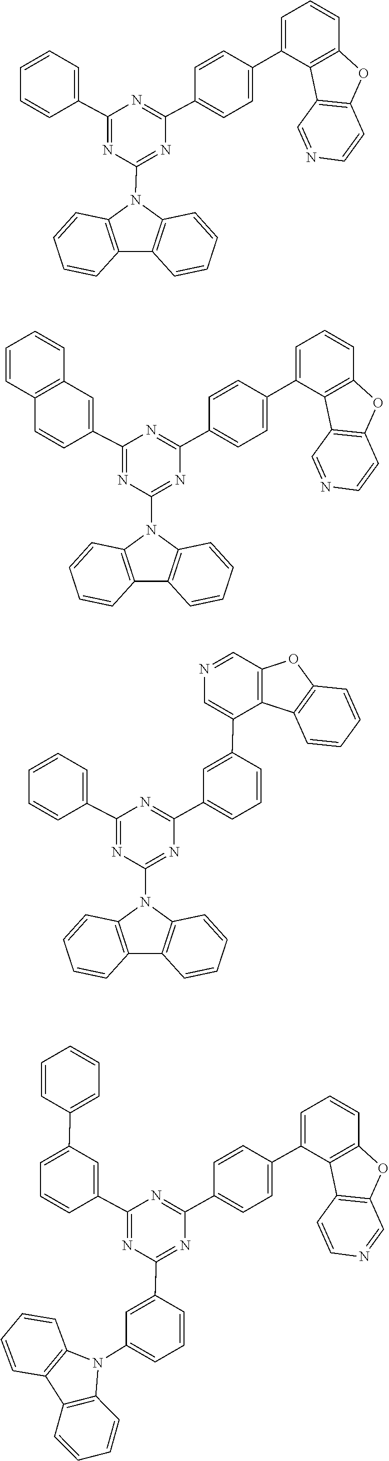 [SMILES: c1ccc(-c2nc(-c3ccc(-c4cccc5oc6ccncc6c45)cc3)nc(-n3c4ccccc4c4ccccc43)n2)cc1 ; c1ccc2cc(-c3nc(-c4ccc(-c5cccc6oc7ccncc7c56)cc4)nc(-n4c5ccccc5c5ccccc54)n3)ccc2c1 ; c1ccc(-c2nc(-c3cccc(-c4cncc5oc6ccccc6c45)c3)nc(-n3c4ccccc4c4ccccc43)n2)cc1 ; c1ccc(-c2cccc(-c3nc(-c4ccc(-c5cccc6oc7cnccc7c56)cc4)nc(-c4cccc(-n5c6ccccc6c6ccccc65)c4)n3)c2)cc1]

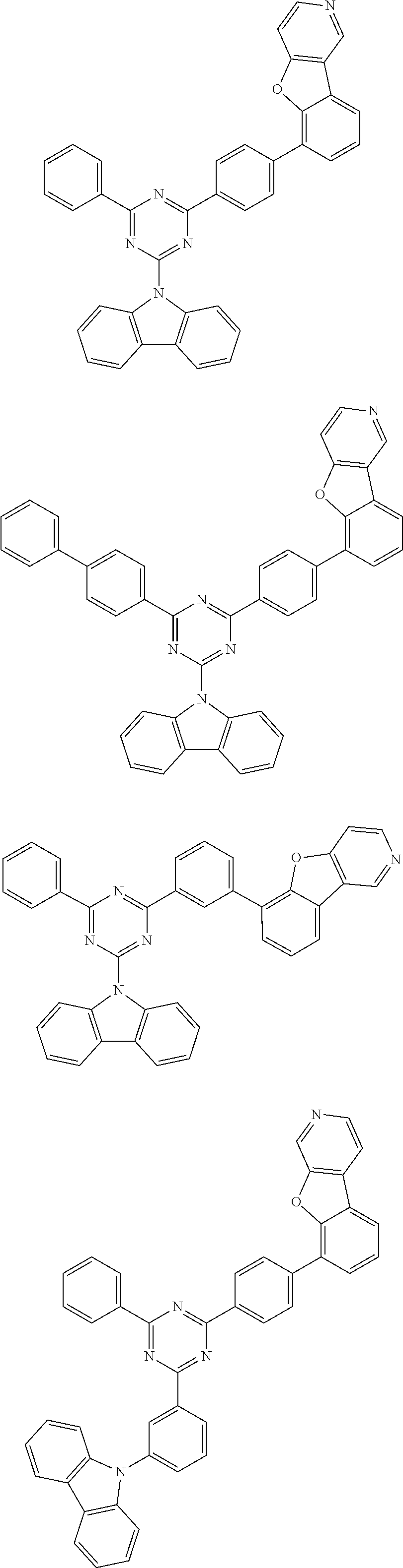 [SMILES: c1ccc(-c2nc(-c3ccc(-c4cccc5c4oc4ccncc45)cc3)nc(-n3c4ccccc4c4ccccc43)n2)cc1 ; c1ccc(-c2ccc(-c3nc(-c4ccc(-c5cccc6c5oc5ccncc56)cc4)nc(-n4c5ccccc5c5ccccc54)n3)cc2)cc1 ; c1ccc(-c2nc(-c3cccc(-c4cccc5c4oc4ccncc45)c3)nc(-n3c4ccccc4c4ccccc43)n2)cc1 ; c1ccc(-c2nc(-c3ccc(-c4cccc5c4oc4cnccc45)cc3)nc(-c3cccc(-n4c5ccccc5c5ccccc54)c3)n2)cc1]

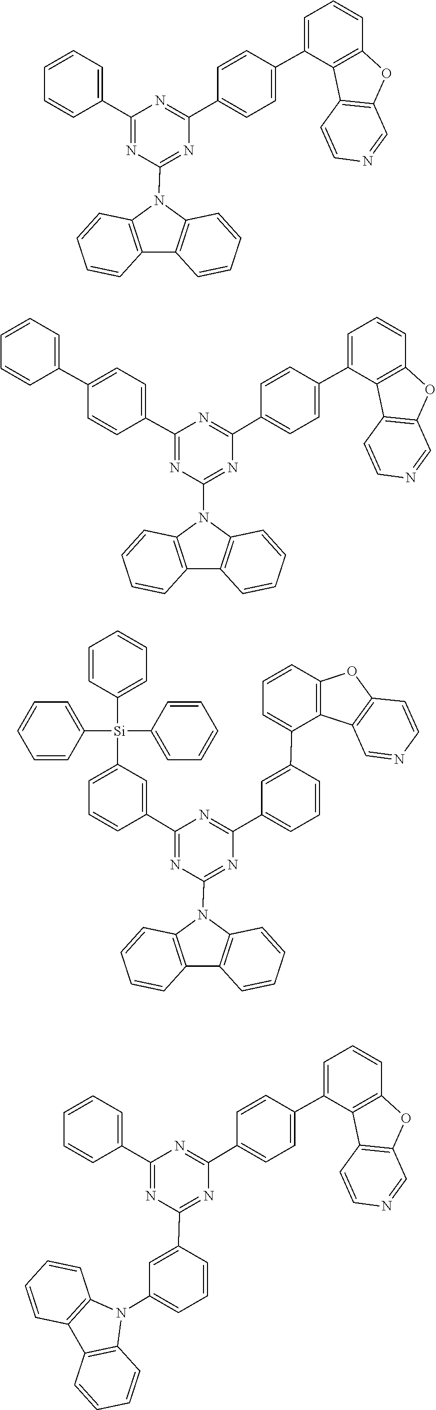

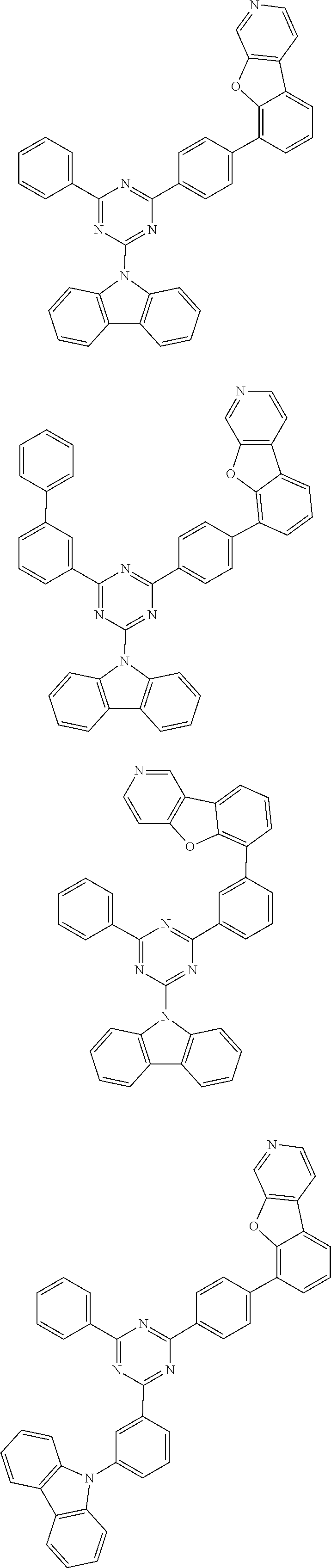

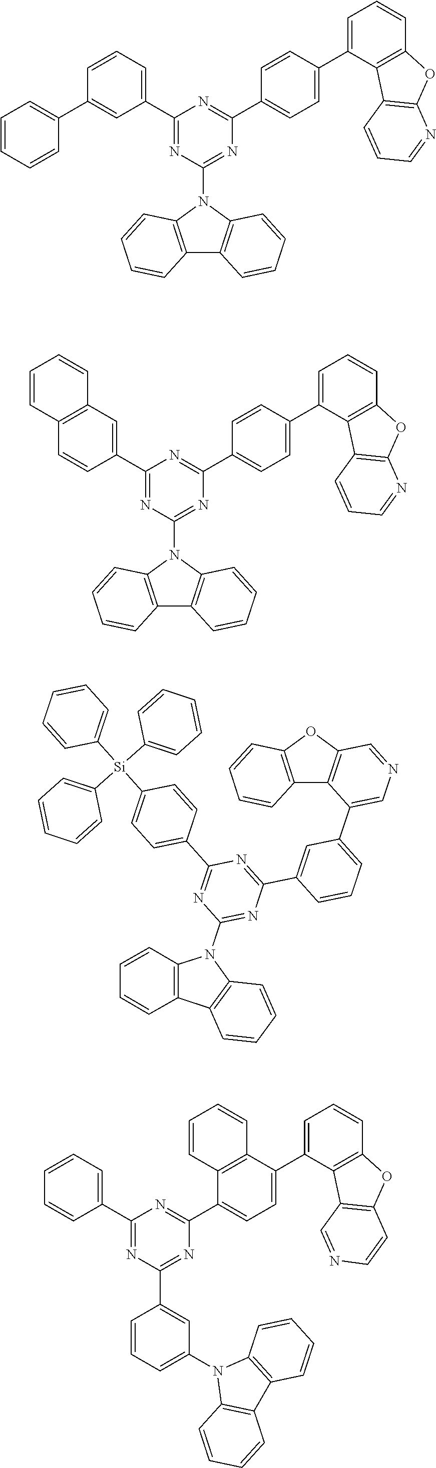 [SMILES: c1ccc(-c2cccc(-c3nc(-c4ccc(-c5cccc6oc7ncccc7c56)cc4)nc(-n4c5ccccc5c5ccccc54)n3)c2)cc1 ; c1ccc2cc(-c3nc(-c4ccc(-c5cccc6oc7ncccc7c56)cc4)nc(-n4c5ccccc5c5ccccc54)n3)ccc2c1 ; c1ccc([Si](c2ccccc2)(c2ccccc2)c2ccc(-c3nc(-c4cccc(-c5cncc6oc7ccccc7c56)c4)nc(-n4c5ccccc5c5ccccc54)n3)cc2)cc1 ; c1ccc(-c2nc(-c3cccc(-n4c5ccccc5c5ccccc54)c3)nc(-c3ccc(-c4cccc5oc6ccncc6c45)c4ccccc34)n2)cc1]

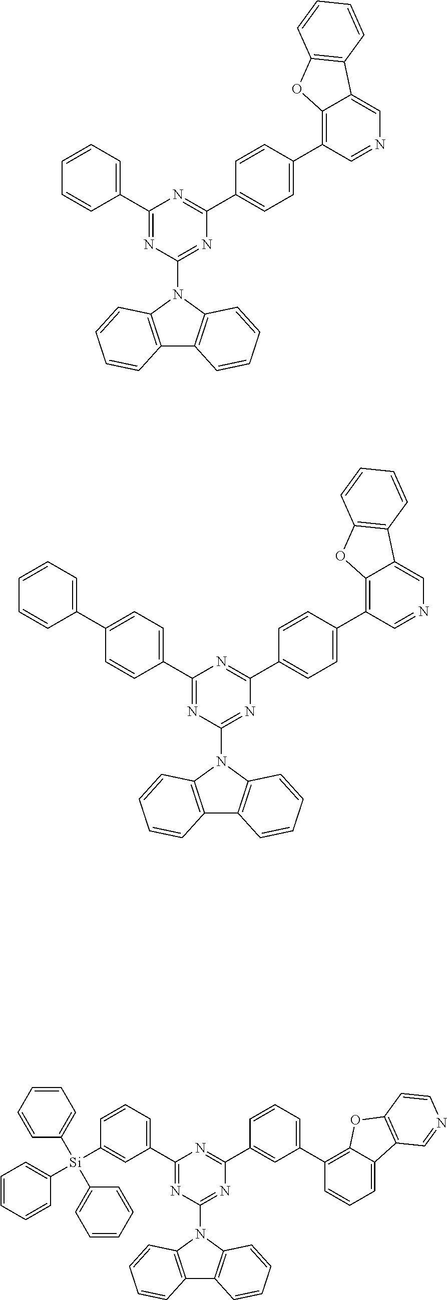 [SMILES: c1ccc(-c2nc(-c3ccc(-c4cncc5c4oc4ccccc45)cc3)nc(-n3c4ccccc4c4ccccc43)n2)cc1 ; c1ccc(-c2ccc(-c3nc(-c4ccc(-c5cncc6c5oc5ccccc56)cc4)nc(-n4c5ccccc5c5ccccc54)n3)cc2)cc1 ; c1ccc([Si](c2ccccc2)(c2ccccc2)c2cccc(-c3nc(-c4cccc(-c5cccc6c5oc5ccncc56)c4)nc(-n4c5ccccc5c5ccccc54)n3)c2)cc1]

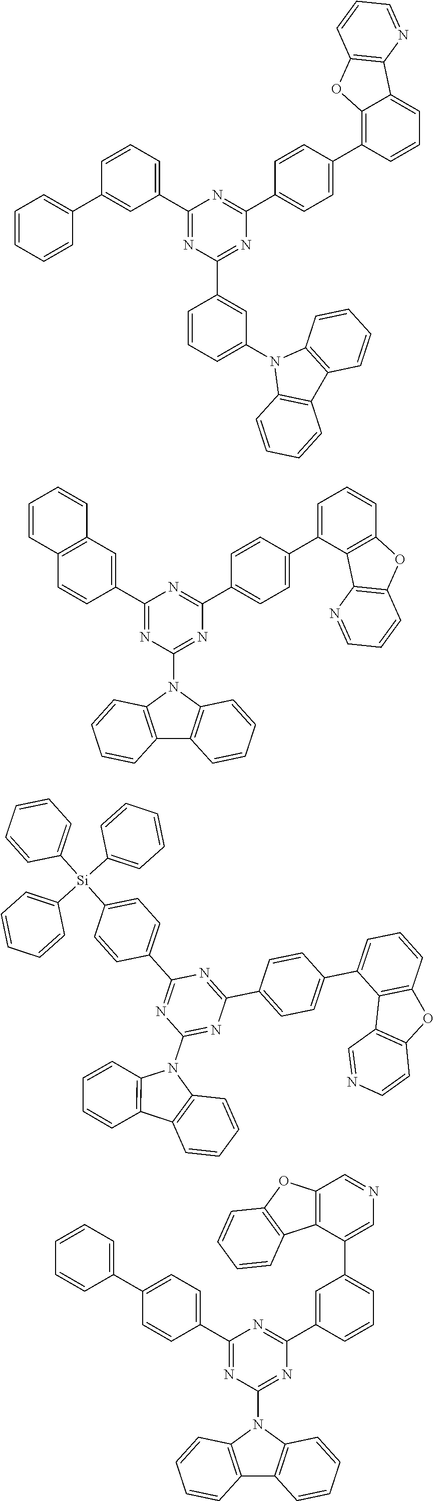

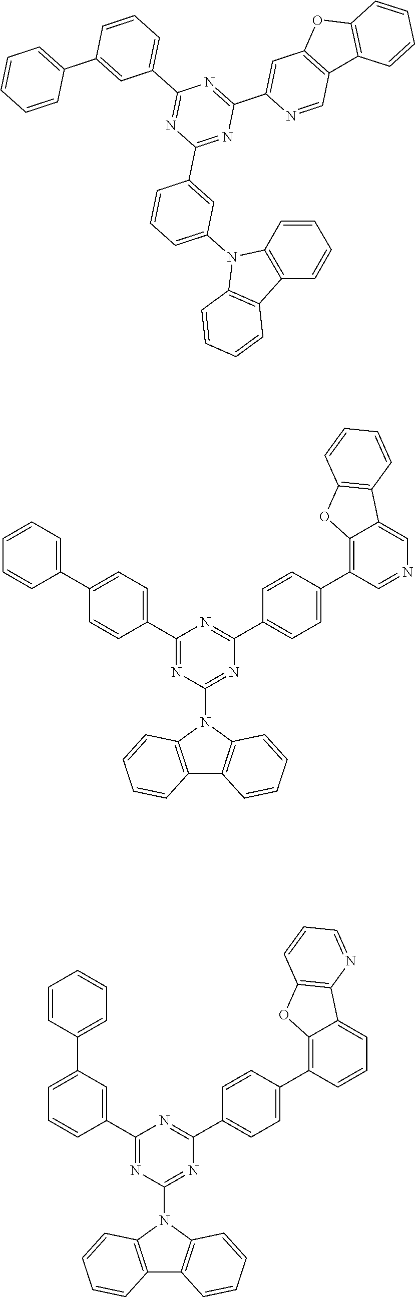 [SMILES: c1ccc(-c2cccc(-c3nc(-c4cccc(-n5c6ccccc6c6ccccc65)c4)nc(-c4cc5oc6ccccc6c5cn4)n3)c2)cc1 ; c1ccc(-c2ccc(-c3nc(-c4ccc(-c5cncc6c5oc5ccccc56)cc4)nc(-n4c5ccccc5c5ccccc54)n3)cc2)cc1 ; c1ccc(-c2cccc(-c3nc(-c4ccc(-c5cccc6c5oc5cccnc56)cc4)nc(-n4c5ccccc5c5ccccc54)n3)c2)cc1]

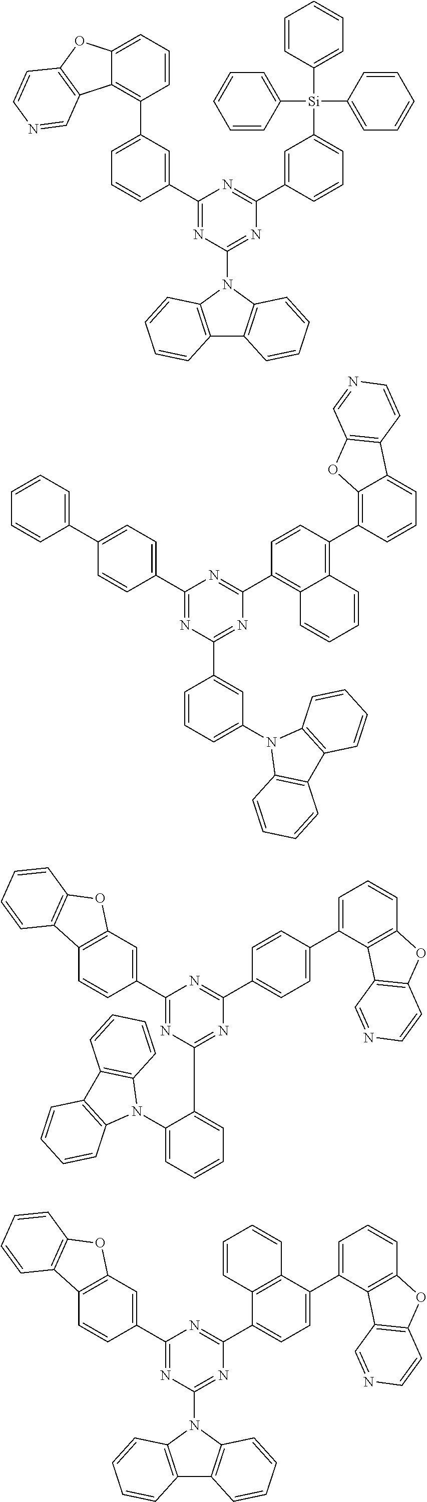 [SMILES: c1ccc([Si](c2ccccc2)(c2ccccc2)c2cccc(-c3nc(-c4cccc(-c5cccc6oc7ccncc7c56)c4)nc(-n4c5ccccc5c5ccccc54)n3)c2)cc1 ; c1ccc(-c2ccc(-c3nc(-c4cccc(-n5c6ccccc6c6ccccc65)c4)nc(-c4ccc(-c5cccc6c5oc5cnccc56)c5ccccc45)n3)cc2)cc1 ; c1ccc(-n2c3ccccc3c3ccccc32)c(-c2nc(-c3ccc(-c4cccc5oc6ccncc6c45)cc3)nc(-c3ccc4c(c3)oc3ccccc34)n2)c1 ; c1ccc2c(c1)oc1cc(-c3nc(-c4ccc(-c5cccc6oc7ccncc7c56)c5ccccc45)nc(-n4c5ccccc5c5ccccc54)n3)ccc12]

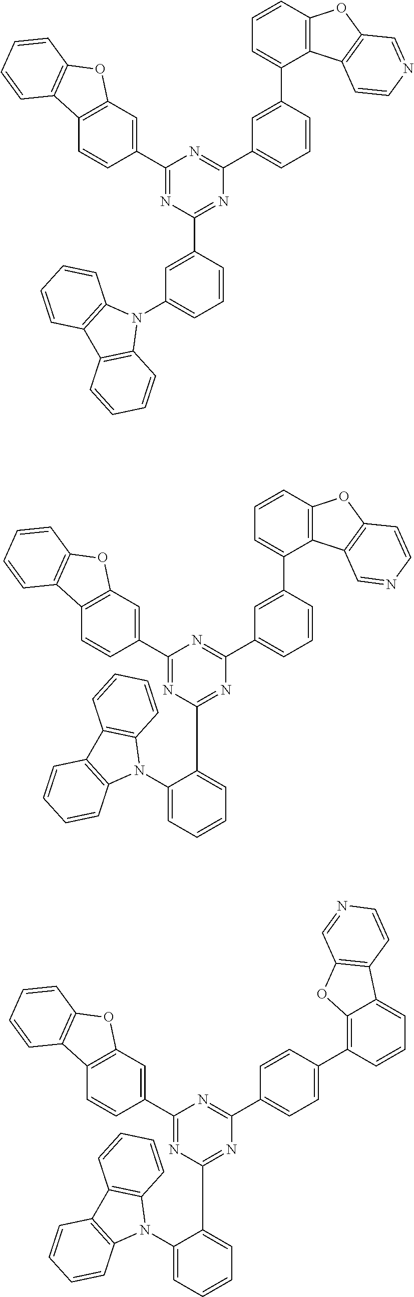 [SMILES: c1cc(-c2nc(-c3cccc(-n4c5ccccc5c5ccccc54)c3)nc(-c3ccc4c(c3)oc3ccccc34)n2)cc(-c2cccc3oc4cnccc4c23)c1 ; c1cc(-c2nc(-c3ccc4c(c3)oc3ccccc34)nc(-c3ccccc3-n3c4ccccc4c4ccccc43)n2)cc(-c2cccc3oc4ccncc4c23)c1 ; c1ccc(-n2c3ccccc3c3ccccc32)c(-c2nc(-c3ccc(-c4cccc5c4oc4cnccc45)cc3)nc(-c3ccc4c(c3)oc3ccccc34)n2)c1]

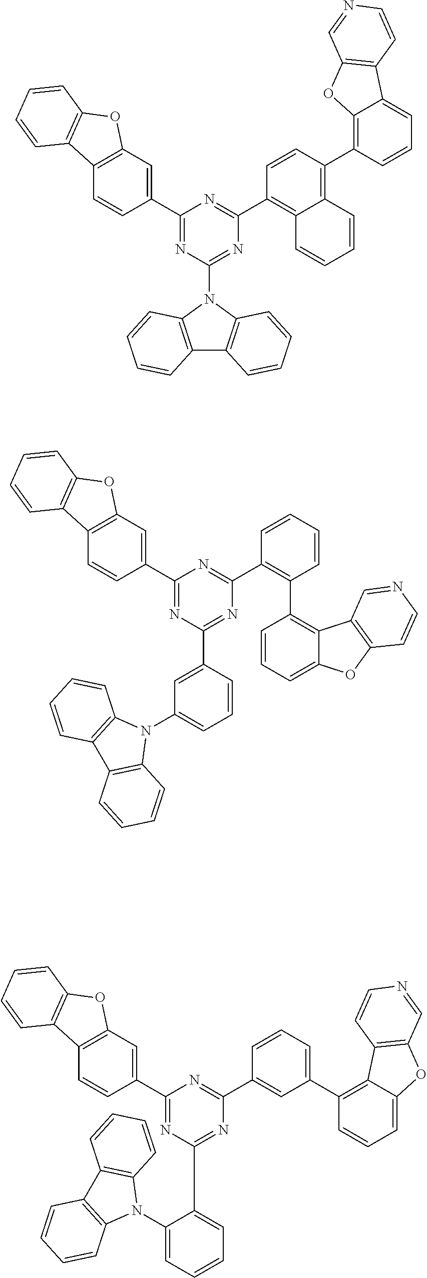 [SMILES: c1ccc2c(c1)oc1cc(-c3nc(-c4ccc(-c5cccc6c5oc5cnccc56)c5ccccc45)nc(-n4c5ccccc5c5ccccc54)n3)ccc12 ; c1cc(-c2nc(-c3ccc4c(c3)oc3ccccc34)nc(-c3ccccc3-c3cccc4oc5ccncc5c34)n2)cc(-n2c3ccccc3c3ccccc32)c1 ; c1cc(-c2nc(-c3ccc4c(c3)oc3ccccc34)nc(-c3ccccc3-n3c4ccccc4c4ccccc43)n2)cc(-c2cccc3oc4cnccc4c23)c1]

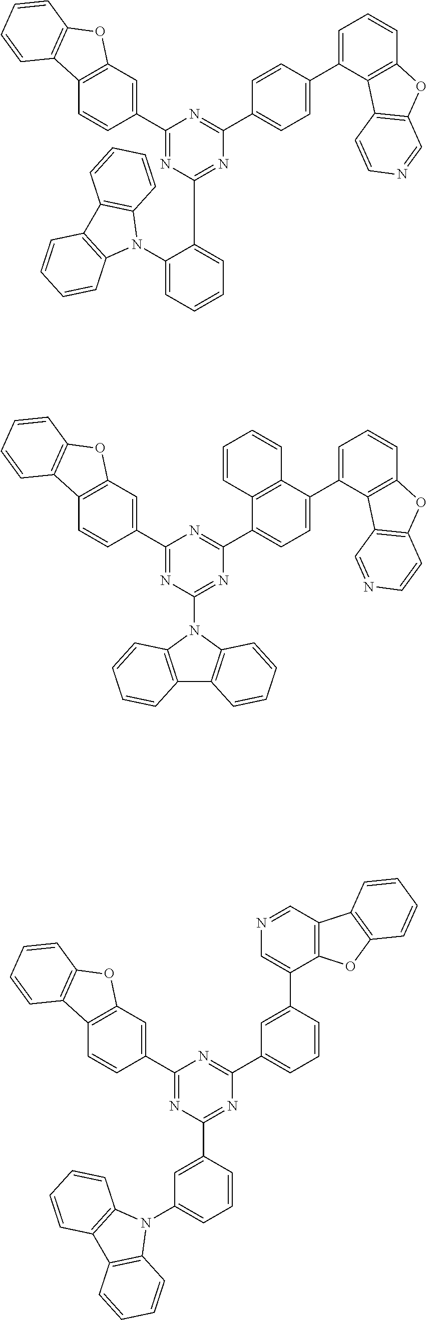 [SMILES: c1ccc(-n2c3ccccc3c3ccccc32)c(-c2nc(-c3ccc(-c4cccc5oc6cnccc6c45)cc3)nc(-c3ccc4c(c3)oc3ccccc34)n2)c1 ; c1ccc2c(c1)oc1cc(-c3nc(-c4ccc(-c5cccc6oc7ccncc7c56)c5ccccc45)nc(-n4c5ccccc5c5ccccc54)n3)ccc12 ; c1cc(-c2nc(-c3cccc(-n4c5ccccc5c5ccccc54)c3)nc(-c3ccc4c(c3)oc3ccccc34)n2)cc(-c2cncc3c2oc2ccccc23)c1]

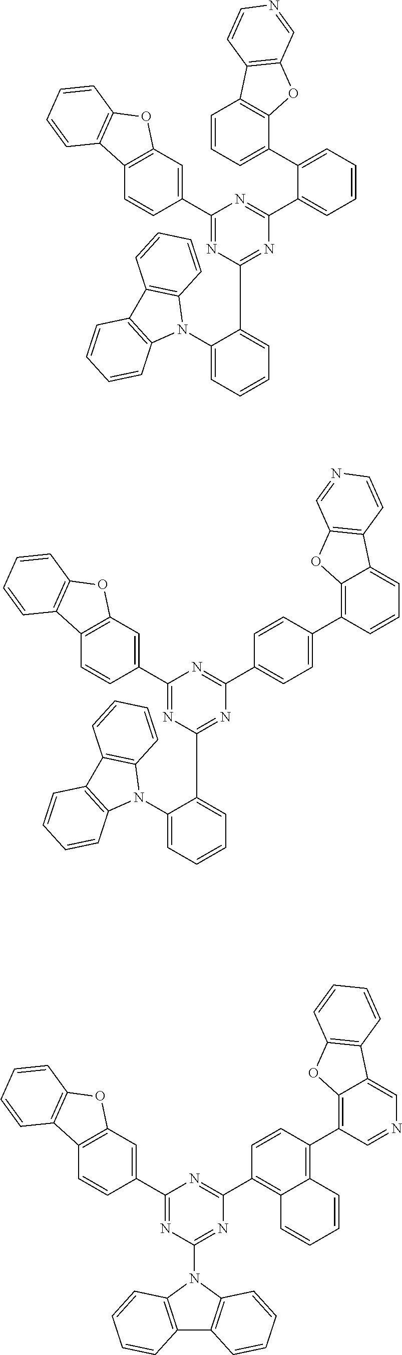 [SMILES: c1ccc(-c2cccc3c2oc2cnccc23)c(-c2nc(-c3ccc4c(c3)oc3ccccc34)nc(-c3ccccc3-n3c4ccccc4c4ccccc43)n2)c1 ; c1ccc(-n2c3ccccc3c3ccccc32)c(-c2nc(-c3ccc(-c4cccc5c4oc4cnccc45)cc3)nc(-c3ccc4c(c3)oc3ccccc34)n2)c1 ; c1ccc2c(c1)oc1cc(-c3nc(-c4ccc(-c5cncc6c5oc5ccccc56)c5ccccc45)nc(-n4c5ccccc5c5ccccc54)n3)ccc12]

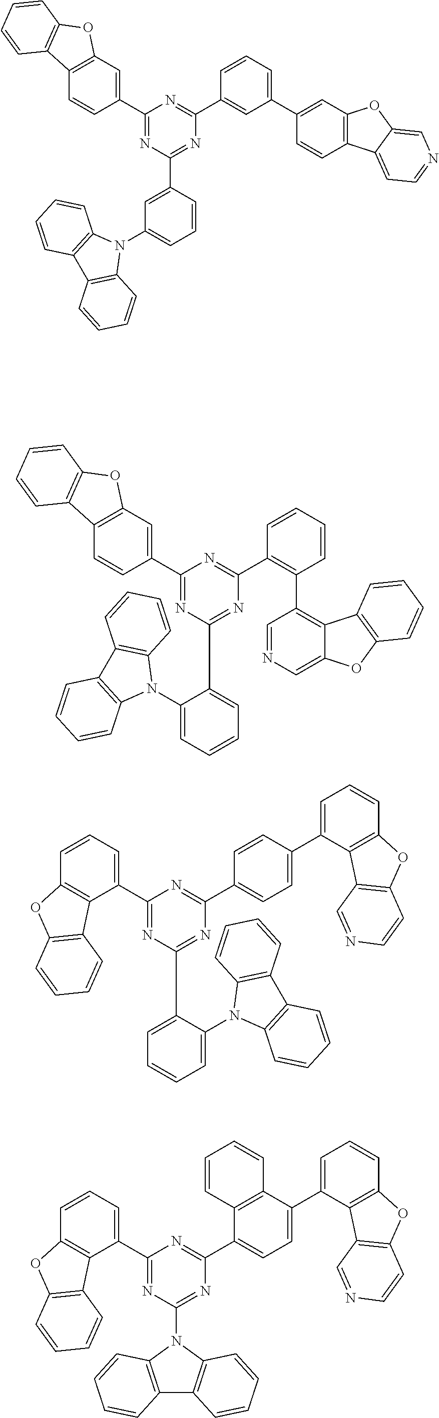 [SMILES: c1cc(-c2ccc3c(c2)oc2cnccc23)cc(-c2nc(-c3cccc(-n4c5ccccc5c5ccccc54)c3)nc(-c3ccc4c(c3)oc3ccccc34)n2)c1 ; c1ccc(-c2cncc3oc4ccccc4c23)c(-c2nc(-c3ccc4c(c3)oc3ccccc34)nc(-c3ccccc3-n3c4ccccc4c4ccccc43)n2)c1 ; c1ccc(-n2c3ccccc3c3ccccc32)c(-c2nc(-c3ccc(-c4cccc5oc6ccncc6c45)cc3)nc(-c3cccc4oc5ccccc5c34)n2)c1 ; c1ccc2c(c1)oc1cccc(-c3nc(-c4ccc(-c5cccc6oc7ccncc7c56)c5ccccc45)nc(-n4c5ccccc5c5ccccc54)n3)c12]

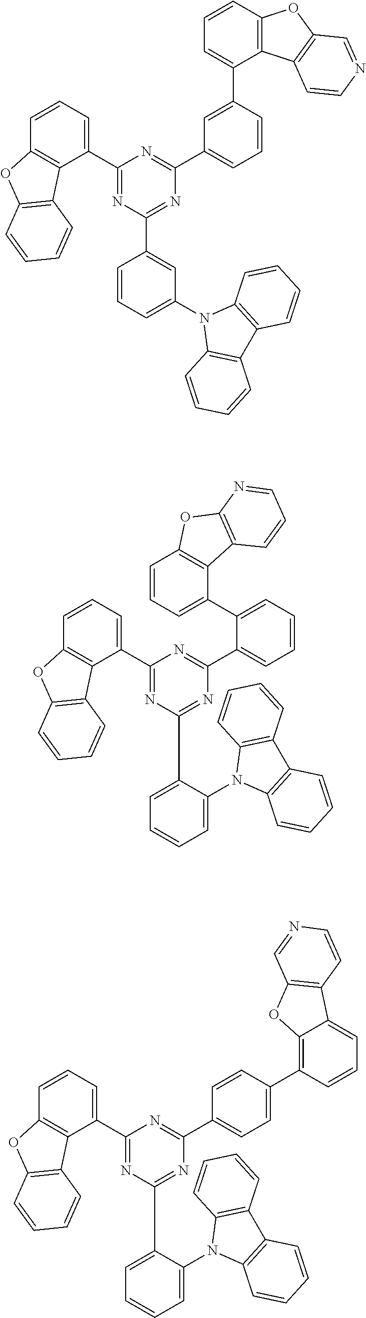 [SMILES: c1cc(-c2nc(-c3cccc(-n4c5ccccc5c5ccccc54)c3)nc(-c3cccc4oc5ccccc5c34)n2)cc(-c2cccc3oc4cnccc4c23)c1 ; c1ccc(-c2cccc3oc4ncccc4c23)c(-c2nc(-c3ccccc3-n3c4ccccc4c4ccccc43)nc(-c3cccc4oc5ccccc5c34)n2)c1 ; c1ccc(-n2c3ccccc3c3ccccc32)c(-c2nc(-c3ccc(-c4cccc5c4oc4cnccc45)cc3)nc(-c3cccc4oc5ccccc5c34)n2)c1]

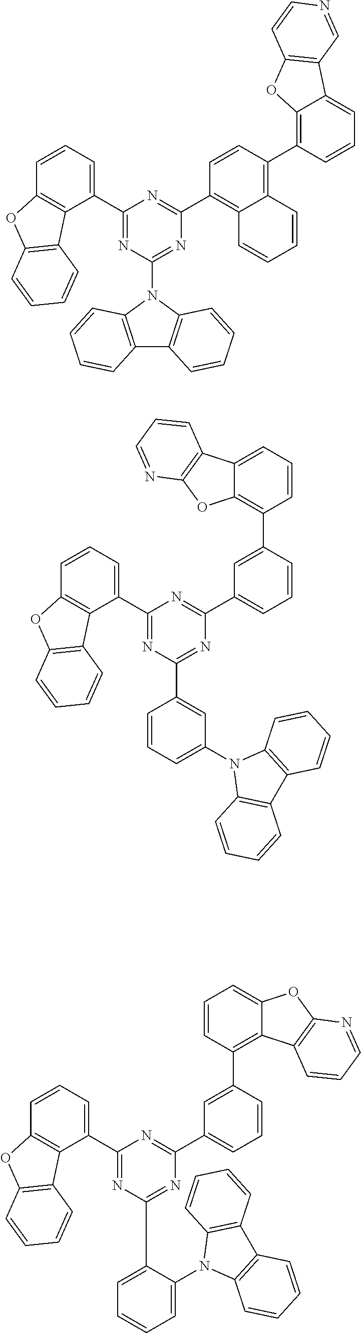 [SMILES: c1ccc2c(c1)oc1cccc(-c3nc(-c4ccc(-c5cccc6c5oc5ccncc56)c5ccccc45)nc(-n4c5ccccc5c5ccccc54)n3)c12 ; c1cc(-c2nc(-c3cccc(-n4c5ccccc5c5ccccc54)c3)nc(-c3cccc4oc5ccccc5c34)n2)cc(-c2cccc3c2oc2ncccc23)c1 ; c1cc(-c2nc(-c3ccccc3-n3c4ccccc4c4ccccc43)nc(-c3cccc4oc5ccccc5c34)n2)cc(-c2cccc3oc4ncccc4c23)c1]

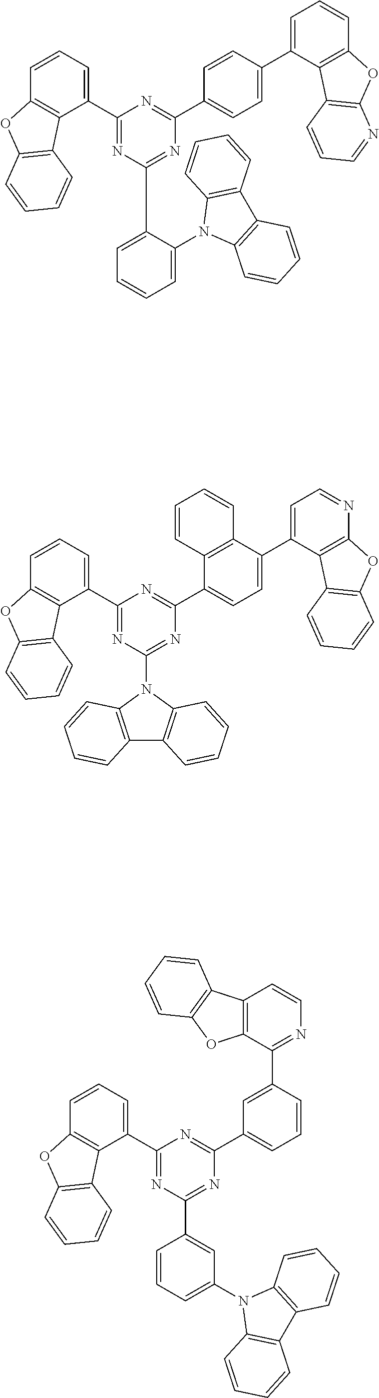 [SMILES: c1ccc(-n2c3ccccc3c3ccccc32)c(-c2nc(-c3ccc(-c4cccc5oc6ncccc6c45)cc3)nc(-c3cccc4oc5ccccc5c34)n2)c1 ; c1ccc2c(c1)oc1cccc(-c3nc(-c4ccc(-c5ccnc6oc7ccccc7c56)c5ccccc45)nc(-n4c5ccccc5c5ccccc54)n3)c12 ; c1cc(-c2nc(-c3cccc(-n4c5ccccc5c5ccccc54)c3)nc(-c3cccc4oc5ccccc5c34)n2)cc(-c2nccc3c2oc2ccccc23)c1]

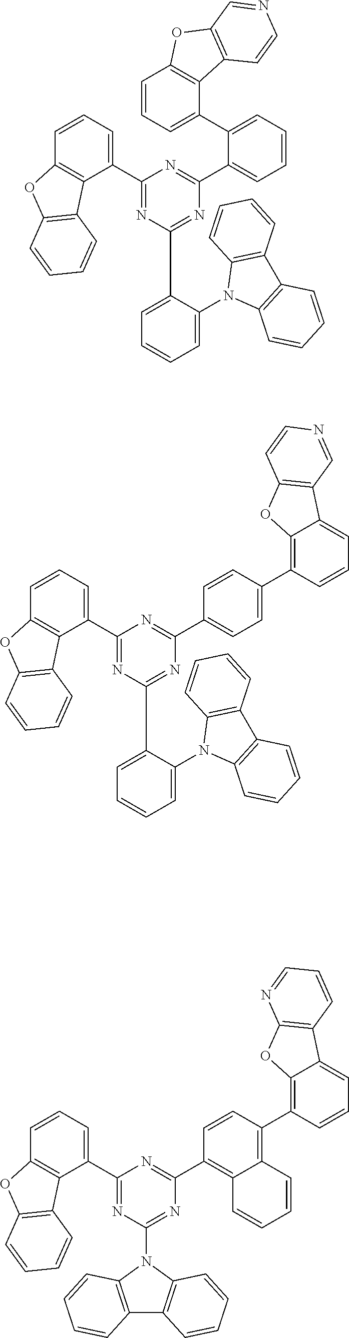 [SMILES: c1ccc(-c2cccc3oc4cnccc4c23)c(-c2nc(-c3ccccc3-n3c4ccccc4c4ccccc43)nc(-c3cccc4oc5ccccc5c34)n2)c1 ; c1ccc(-n2c3ccccc3c3ccccc32)c(-c2nc(-c3ccc(-c4cccc5c4oc4ccncc45)cc3)nc(-c3cccc4oc5ccccc5c34)n2)c1 ; c1ccc2c(c1)oc1cccc(-c3nc(-c4ccc(-c5cccc6c5oc5ncccc56)c5ccccc45)nc(-n4c5ccccc5c5ccccc54)n3)c12]

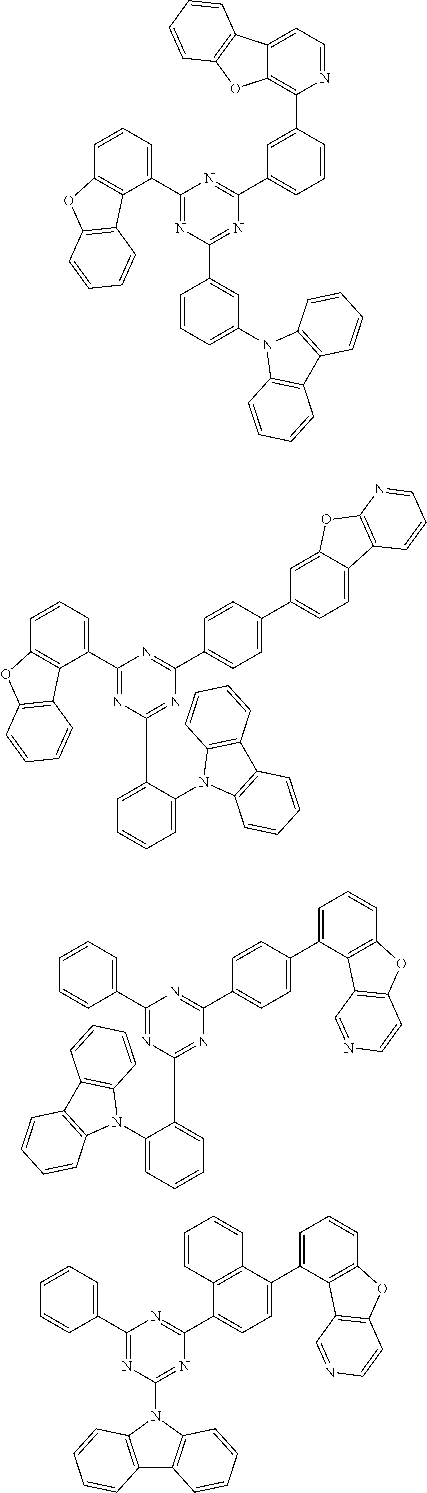

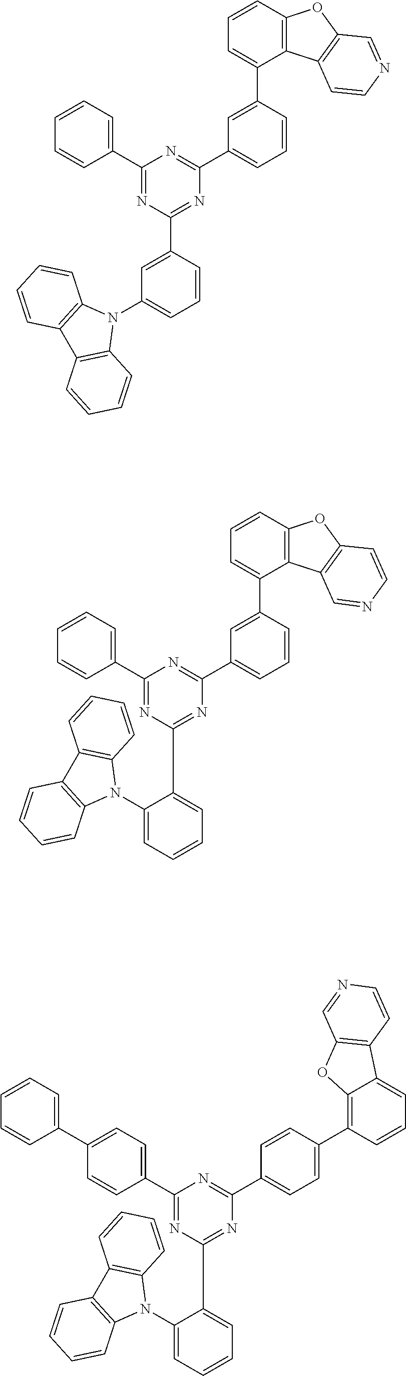 [SMILES: c1ccc(-c2nc(-c3cccc(-c4cccc5oc6cnccc6c45)c3)nc(-c3cccc(-n4c5ccccc5c5ccccc54)c3)n2)cc1 ; c1ccc(-c2nc(-c3cccc(-c4cccc5oc6ccncc6c45)c3)nc(-c3ccccc3-n3c4ccccc4c4ccccc43)n2)cc1 ; c1ccc(-c2ccc(-c3nc(-c4ccc(-c5cccc6c5oc5cnccc56)cc4)nc(-c4ccccc4-n4c5ccccc5c5ccccc54)n3)cc2)cc1]

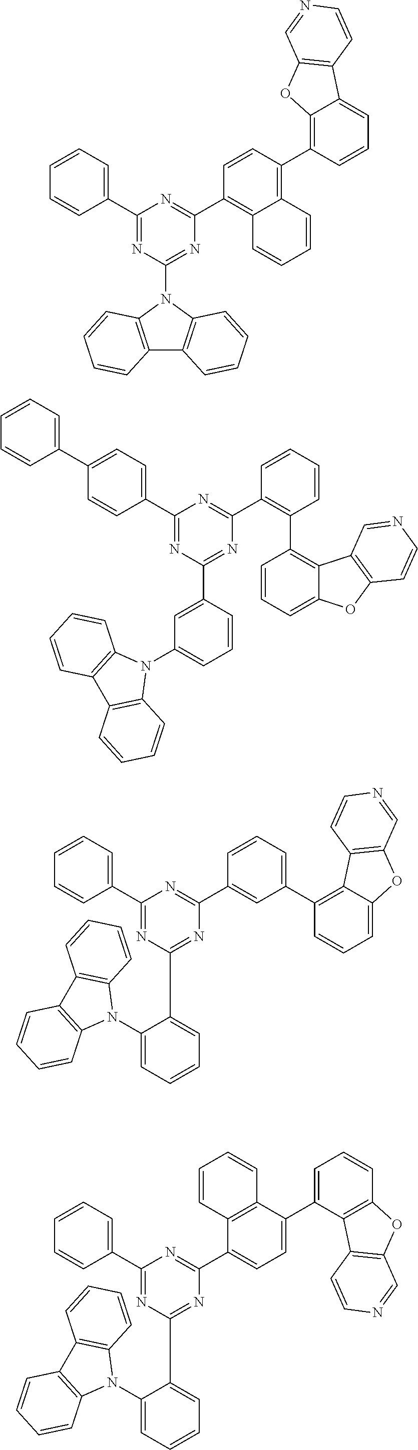 [SMILES: c1ccc(-c2nc(-c3ccc(-c4cccc5c4oc4cnccc45)c4ccccc34)nc(-n3c4ccccc4c4ccccc43)n2)cc1 ; c1ccc(-c2ccc(-c3nc(-c4cccc(-n5c6ccccc6c6ccccc65)c4)nc(-c4ccccc4-c4cccc5oc6ccncc6c45)n3)cc2)cc1 ; c1ccc(-c2nc(-c3cccc(-c4cccc5oc6cnccc6c45)c3)nc(-c3ccccc3-n3c4ccccc4c4ccccc43)n2)cc1 ; c1ccc(-c2nc(-c3ccccc3-n3c4ccccc4c4ccccc43)nc(-c3ccc(-c4cccc5oc6cnccc6c45)c4ccccc34)n2)cc1]

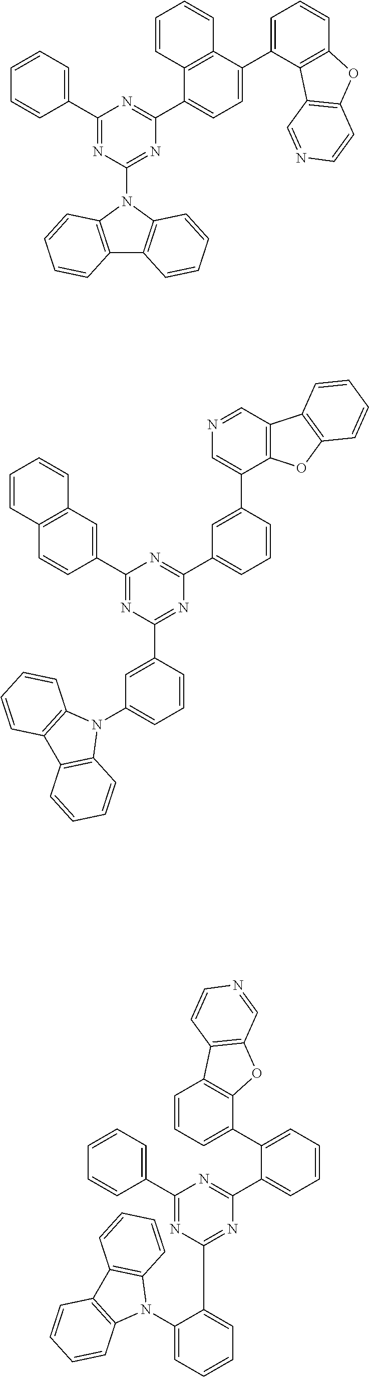 [SMILES: c1ccc(-c2nc(-c3ccc(-c4cccc5oc6ccncc6c45)c4ccccc34)nc(-n3c4ccccc4c4ccccc43)n2)cc1 ; c1cc(-c2nc(-c3cccc(-n4c5ccccc5c5ccccc54)c3)nc(-c3ccc4ccccc4c3)n2)cc(-c2cncc3c2oc2ccccc23)c1 ; c1ccc(-c2nc(-c3ccccc3-c3cccc4c3oc3cnccc34)nc(-c3ccccc3-n3c4ccccc4c4ccccc43)n2)cc1]

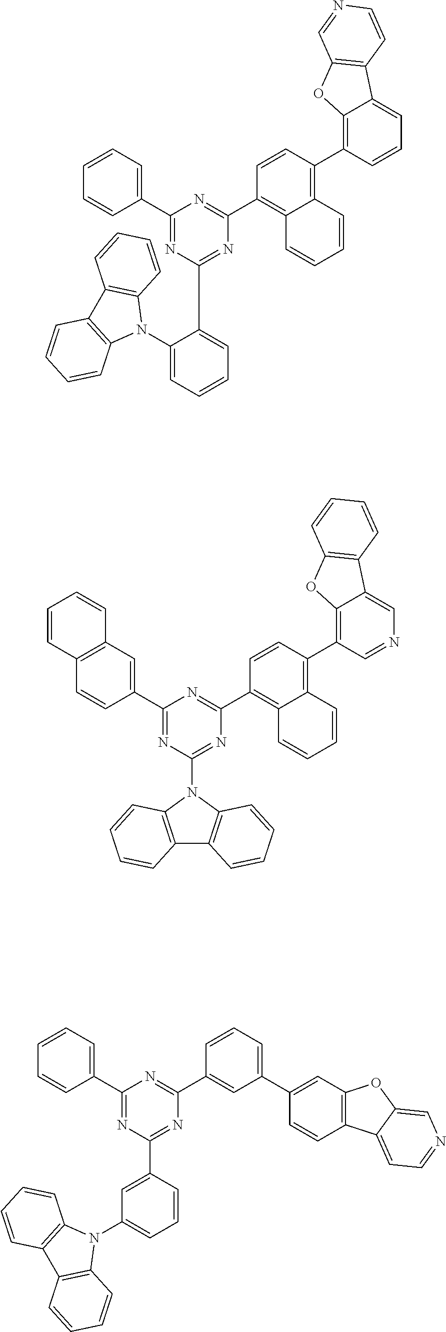 [SMILES: c1ccc(-c2nc(-c3ccccc3-n3c4ccccc4c4ccccc43)nc(-c3ccc(-c4cccc5c4oc4cnccc45)c4ccccc34)n2)cc1 ; c1ccc2cc(-c3nc(-c4ccc(-c5cncc6c5oc5ccccc56)c5ccccc45)nc(-n4c5ccccc5c5ccccc54)n3)ccc2c1 ; c1ccc(-c2nc(-c3cccc(-c4ccc5c(c4)oc4cnccc45)c3)nc(-c3cccc(-n4c5ccccc5c5ccccc54)c3)n2)cc1]

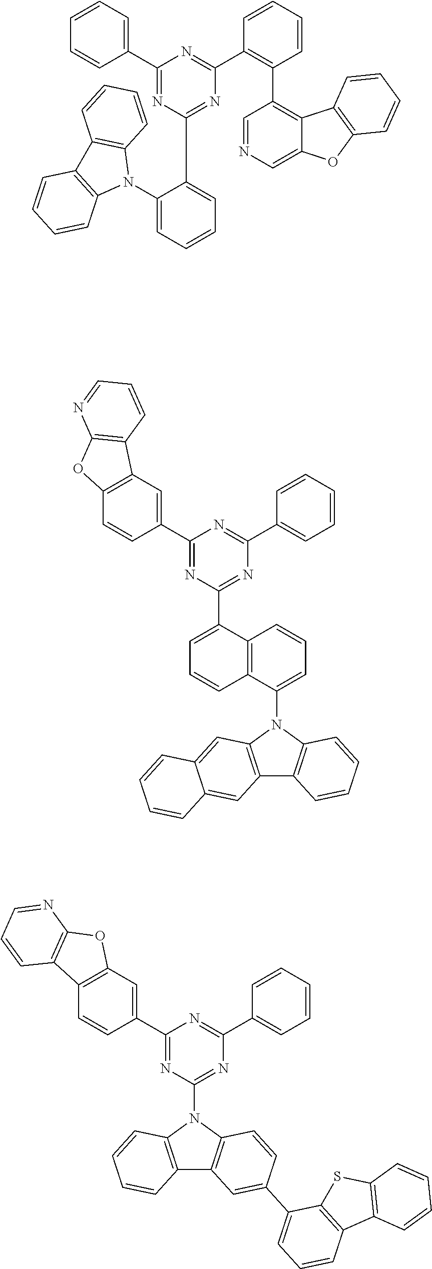 [SMILES: c1ccc(-c2nc(-c3ccccc3-c3cncc4oc5ccccc5c34)nc(-c3ccccc3-n3c4ccccc4c4ccccc43)n2)cc1 ; c1ccc(-c2nc(-c3ccc4oc5ncccc5c4c3)nc(-c3cccc4c(-n5c6ccccc6c6cc7ccccc7cc65)cccc34)n2)cc1 ; c1ccc(-c2nc(-c3ccc4c(c3)oc3ncccc34)nc(-n3c4ccccc4c4cc(-c5cccc6c5sc5ccccc56)ccc43)n2)cc1]

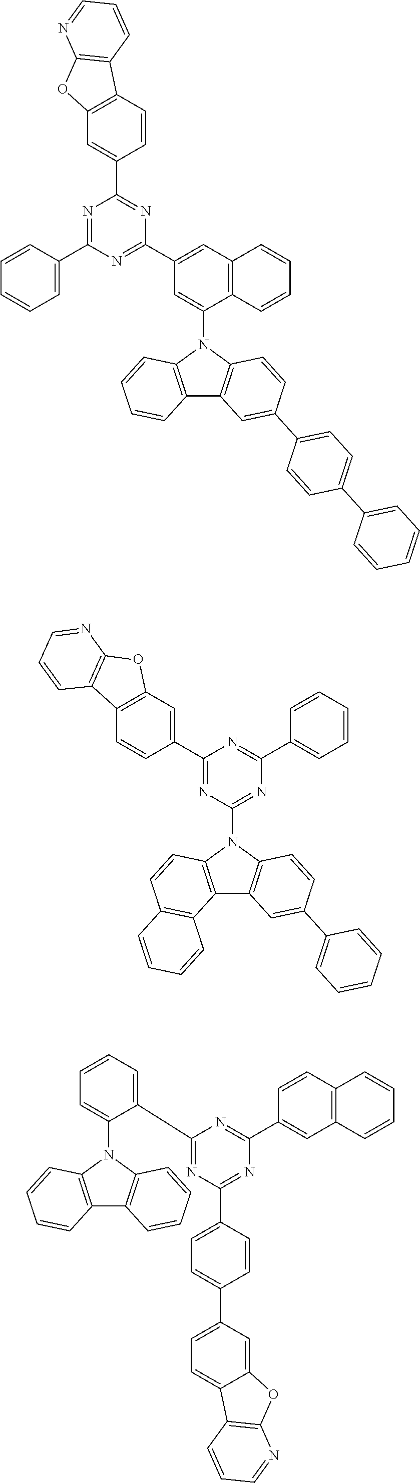 [SMILES: c1ccc(-c2ccc(-c3ccc4c(c3)c3ccccc3n4-c3cc(-c4nc(-c5ccccc5)nc(-c5ccc6c(c5)oc5ncccc56)n4)cc4ccccc34)cc2)cc1 ; c1ccc(-c2ccc3c(c2)c2c4ccccc4ccc2n3-c2nc(-c3ccccc3)nc(-c3ccc4c(c3)oc3ncccc34)n2)cc1 ; c1ccc(-n2c3ccccc3c3ccccc32)c(-c2nc(-c3ccc(-c4ccc5c(c4)oc4ncccc45)cc3)nc(-c3ccc4ccccc4c3)n2)c1]

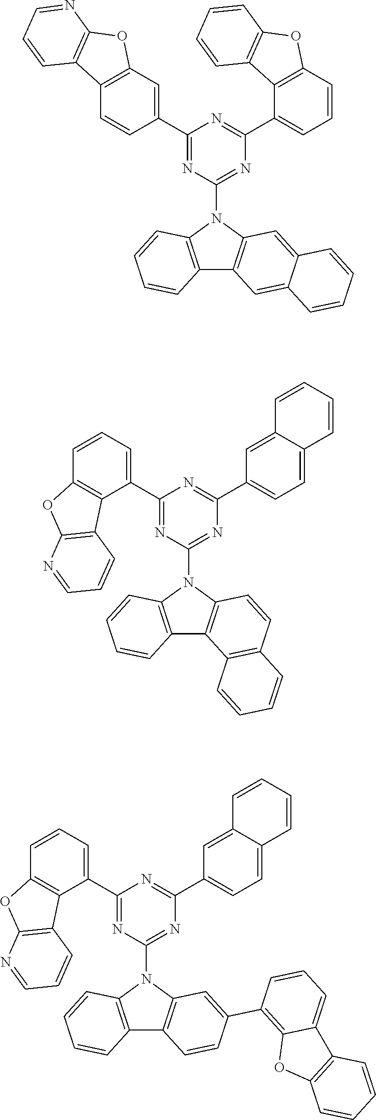 [SMILES: c1ccc2cc3c(cc2c1)c1ccccc1n3-c1nc(-c2ccc3c(c2)oc2ncccc23)nc(-c2cccc3oc4ccccc4c23)n1 ; c1ccc2cc(-c3nc(-c4cccc5oc6ncccc6c45)nc(-n4c5ccccc5c5c6ccccc6ccc54)n3)ccc2c1 ; c1ccc2cc(-c3nc(-c4cccc5oc6ncccc6c45)nc(-n4c5ccccc5c5ccc(-c6cccc7c6oc6ccccc67)cc54)n3)ccc2c1]

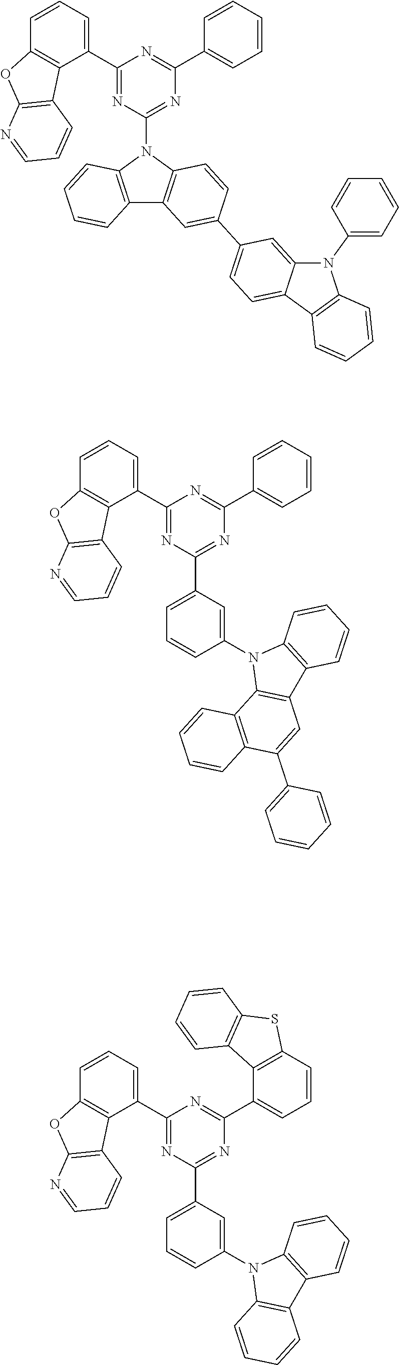 [SMILES: c1ccc(-c2nc(-c3cccc4oc5ncccc5c34)nc(-n3c4ccccc4c4cc(-c5ccc6c7ccccc7n(-c7ccccc7)c6c5)ccc43)n2)cc1 ; c1ccc(-c2nc(-c3cccc(-n4c5ccccc5c5cc(-c6ccccc6)c6ccccc6c54)c3)nc(-c3cccc4oc5ncccc5c34)n2)cc1 ; c1cc(-c2nc(-c3cccc4oc5ncccc5c34)nc(-c3cccc4sc5ccccc5c34)n2)cc(-n2c3ccccc3c3ccccc32)c1]

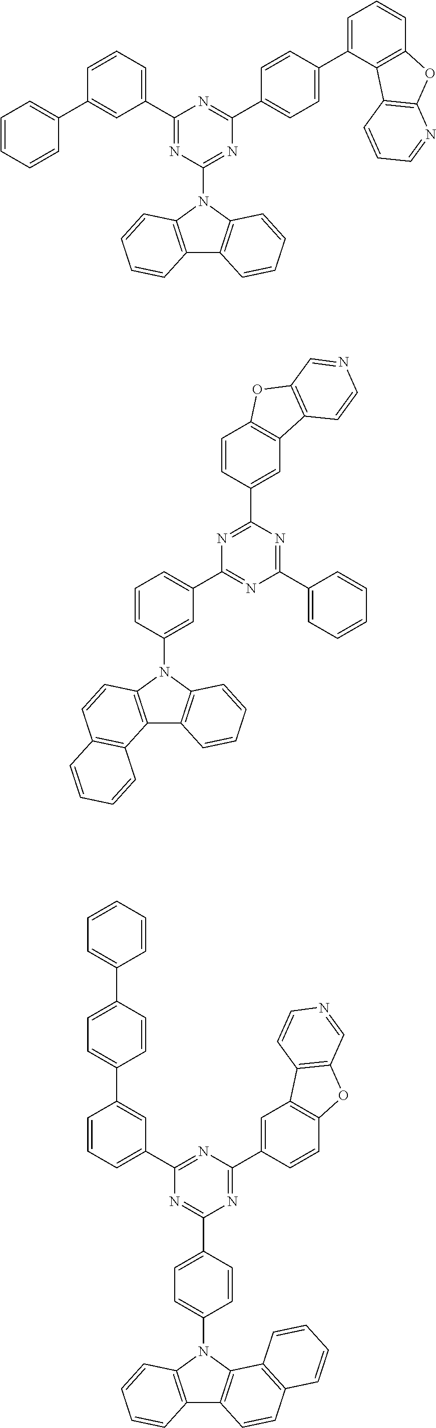 [SMILES: c1ccc(-c2cccc(-c3nc(-c4ccc(-c5cccc6oc7ncccc7c56)cc4)nc(-n4c5ccccc5c5ccccc54)n3)c2)cc1 ; c1ccc(-c2nc(-c3cccc(-n4c5ccccc5c5c6ccccc6ccc54)c3)nc(-c3ccc4oc5cnccc5c4c3)n2)cc1 ; c1ccc(-c2ccc(-c3cccc(-c4nc(-c5ccc(-n6c7ccccc7c7ccc8ccccc8c76)cc5)nc(-c5ccc6oc7cnccc7c6c5)n4)c3)cc2)cc1]

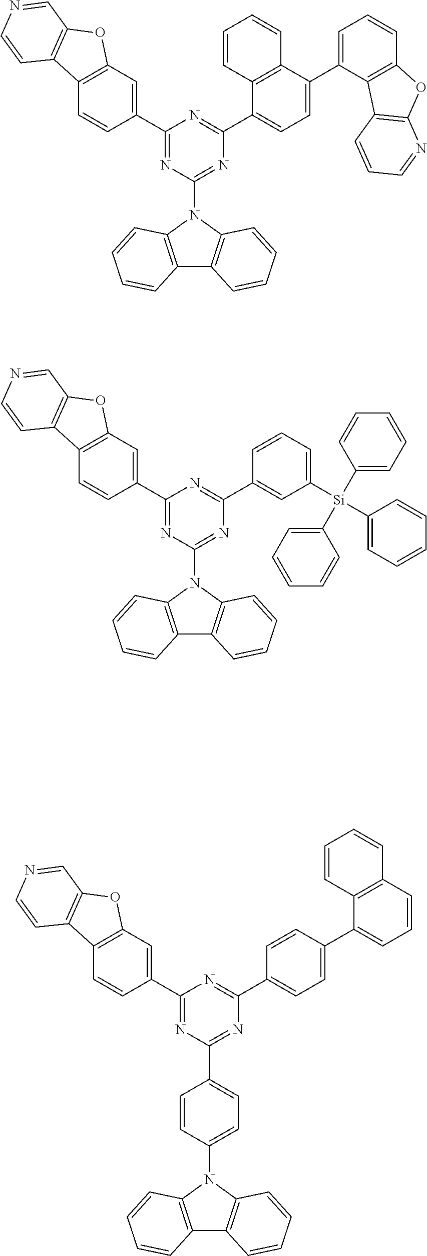 [SMILES: c1cc(-c2ccc(-c3nc(-c4ccc5c(c4)oc4cnccc45)nc(-n4c5ccccc5c5ccccc54)n3)c3ccccc23)c2c(c1)oc1ncccc12 ; c1ccc([Si](c2ccccc2)(c2ccccc2)c2cccc(-c3nc(-c4ccc5c(c4)oc4cnccc45)nc(-n4c5ccccc5c5ccccc54)n3)c2)cc1 ; c1ccc2c(-c3ccc(-c4nc(-c5ccc(-n6c7ccccc7c7ccccc76)cc5)nc(-c5ccc6c(c5)oc5cnccc56)n4)cc3)cccc2c1]

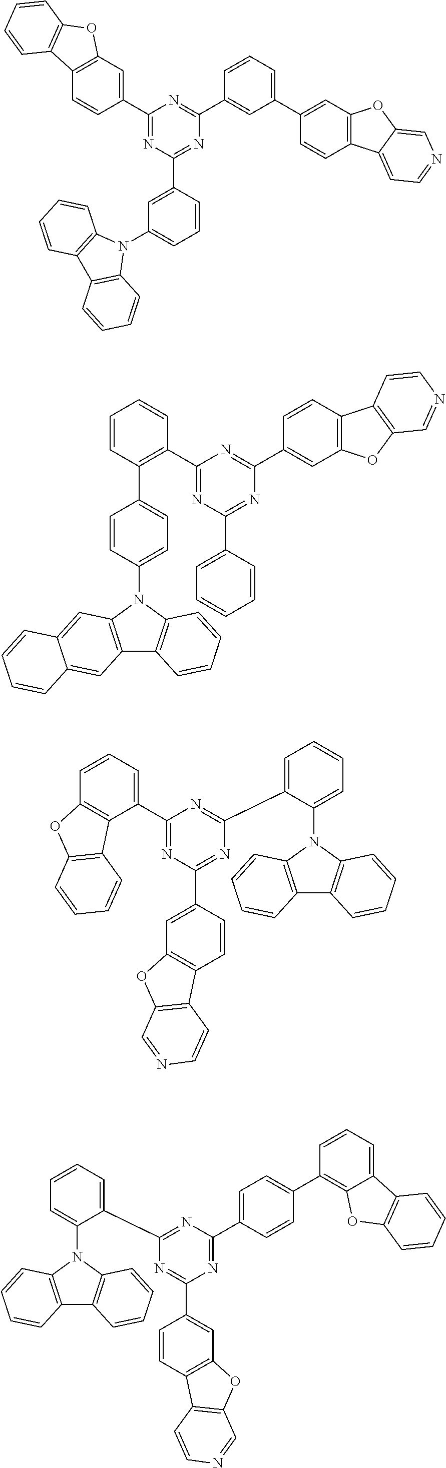 [SMILES: c1cc(-c2ccc3c(c2)oc2cnccc23)cc(-c2nc(-c3cccc(-n4c5ccccc5c5ccccc54)c3)nc(-c3ccc4c(c3)oc3ccccc34)n2)c1 ; c1ccc(-c2nc(-c3ccc4c(c3)oc3cnccc34)nc(-c3ccccc3-c3ccc(-n4c5ccccc5c5cc6ccccc6cc54)cc3)n2)cc1 ; c1ccc(-n2c3ccccc3c3ccccc32)c(-c2nc(-c3ccc4c(c3)oc3cnccc34)nc(-c3cccc4oc5ccccc5c34)n2)c1 ; c1ccc(-n2c3ccccc3c3ccccc32)c(-c2nc(-c3ccc(-c4cccc5c4oc4ccccc45)cc3)nc(-c3ccc4c(c3)oc3cnccc34)n2)c1]

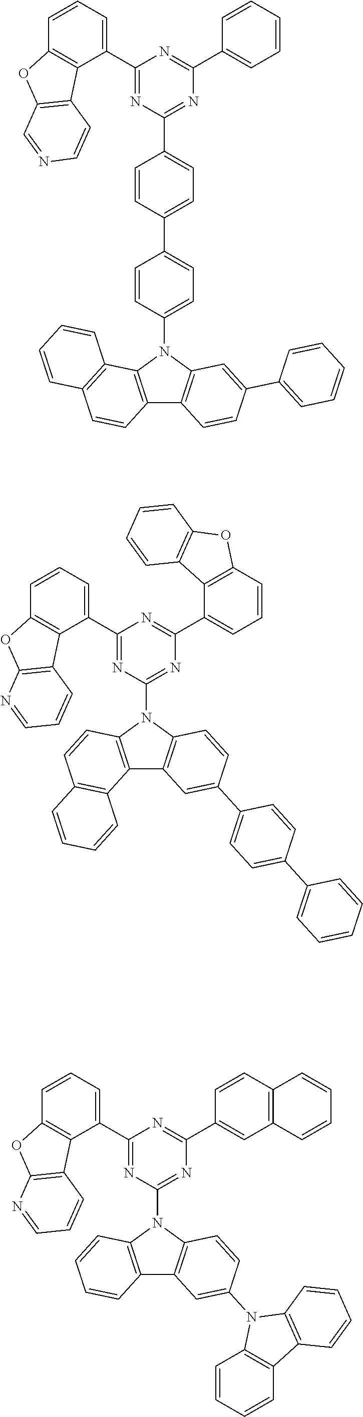 [SMILES: c1ccc(-c2ccc3c4ccc5ccccc5c4n(-c4ccc(-c5ccc(-c6nc(-c7ccccc7)nc(-c7cccc8oc9cnccc9c78)n6)cc5)cc4)c3c2)cc1 ; c1ccc(-c2ccc(-c3ccc4c(c3)c3c5ccccc5ccc3n4-c3nc(-c4cccc5oc6ccccc6c45)nc(-c4cccc5oc6ncccc6c45)n3)cc2)cc1 ; c1ccc2cc(-c3nc(-c4cccc5oc6ncccc6c45)nc(-n4c5ccccc5c5cc(-n6c7ccccc7c7ccccc76)ccc54)n3)ccc2c1]

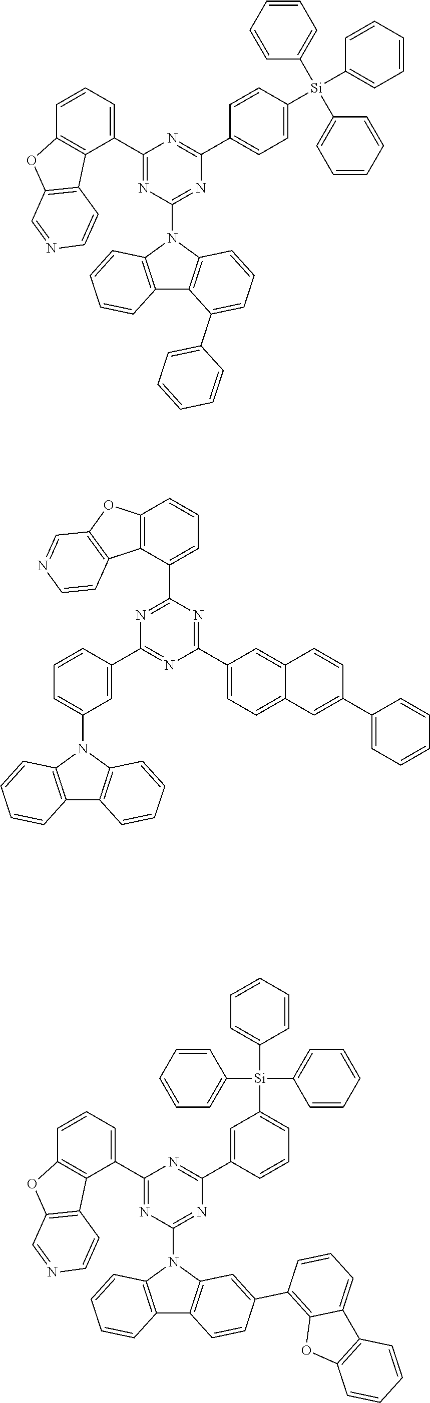 [SMILES: c1ccc(-c2cccc3c2c2ccccc2n3-c2nc(-c3ccc([Si](c4ccccc4)(c4ccccc4)c4ccccc4)cc3)nc(-c3cccc4oc5cnccc5c34)n2)cc1 ; c1ccc(-c2ccc3cc(-c4nc(-c5cccc(-n6c7ccccc7c7ccccc76)c5)nc(-c5cccc6oc7cnccc7c56)n4)ccc3c2)cc1 ; c1ccc([Si](c2ccccc2)(c2ccccc2)c2cccc(-c3nc(-c4cccc5oc6cnccc6c45)nc(-n4c5ccccc5c5ccc(-c6cccc7c6oc6ccccc67)cc54)n3)c2)cc1]

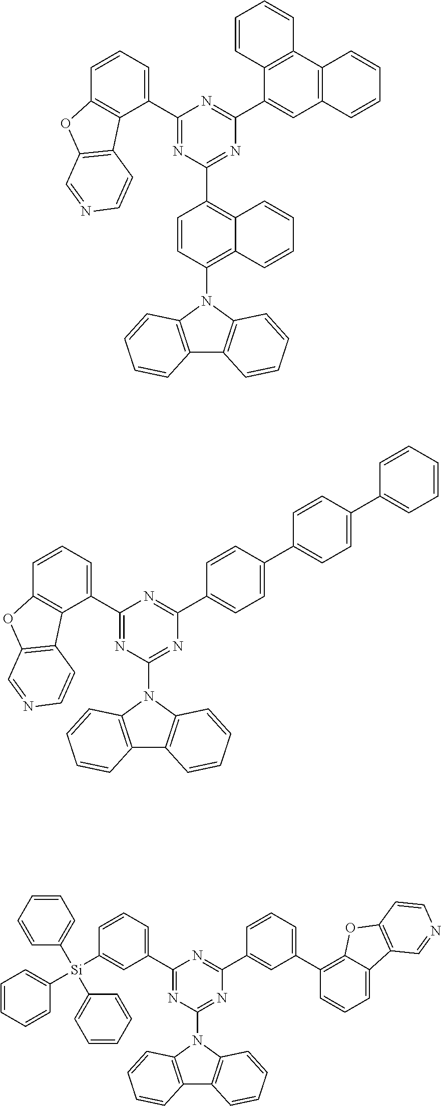 [SMILES: c1ccc2c(c1)cc(-c1nc(-c3ccc(-n4c5ccccc5c5ccccc54)c4ccccc34)nc(-c3cccc4oc5cnccc5c34)n1)c1ccccc12 ; c1ccc(-c2ccc(-c3ccc(-c4nc(-c5cccc6oc7cnccc7c56)nc(-n5c6ccccc6c6ccccc65)n4)cc3)cc2)cc1 ; c1ccc([Si](c2ccccc2)(c2ccccc2)c2cccc(-c3nc(-c4cccc(-c5cccc6c5oc5ccncc56)c4)nc(-n4c5ccccc5c5ccccc54)n3)c2)cc1]

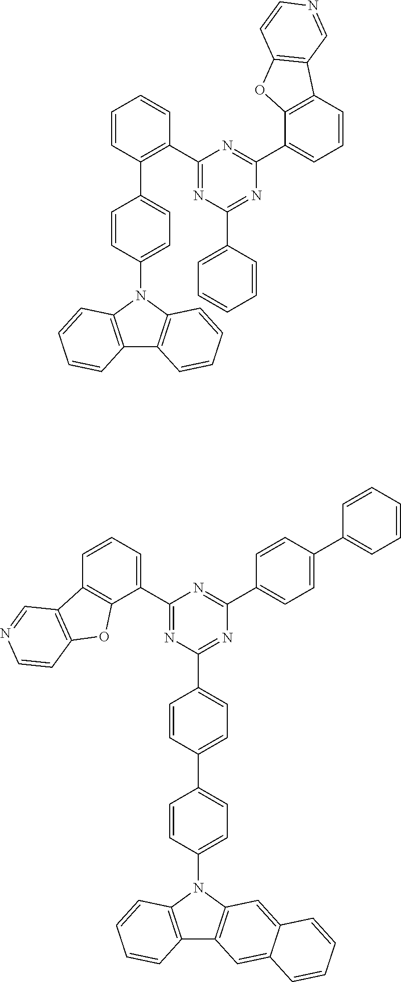 [SMILES: c1ccc(-c2nc(-c3ccccc3-c3ccc(-n4c5ccccc5c5ccccc54)cc3)nc(-c3cccc4c3oc3ccncc34)n2)cc1 ; c1ccc(-c2ccc(-c3nc(-c4ccc(-c5ccc(-n6c7ccccc7c7cc8ccccc8cc76)cc5)cc4)nc(-c4cccc5c4oc4ccncc45)n3)cc2)cc1]

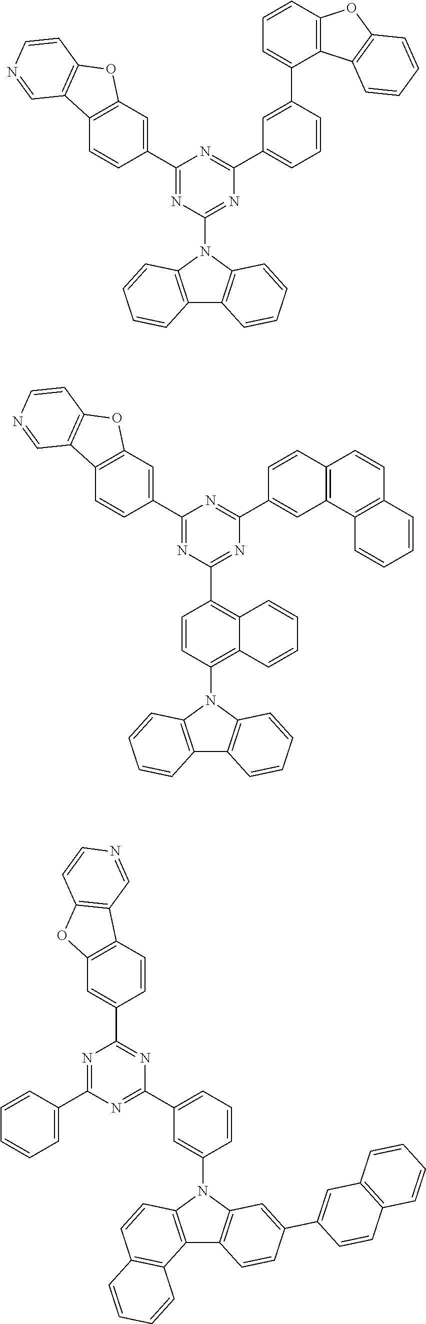 [SMILES: c1cc(-c2nc(-c3ccc4c(c3)oc3ccncc34)nc(-n3c4ccccc4c4ccccc43)n2)cc(-c2cccc3oc4ccccc4c23)c1 ; c1ccc2c(c1)ccc1ccc(-c3nc(-c4ccc5c(c4)oc4ccncc45)nc(-c4ccc(-n5c6ccccc6c6ccccc65)c5ccccc45)n3)cc12 ; c1ccc(-c2nc(-c3cccc(-n4c5cc(-c6ccc7ccccc7c6)ccc5c5c6ccccc6ccc54)c3)nc(-c3ccc4c(c3)oc3ccncc34)n2)cc1]

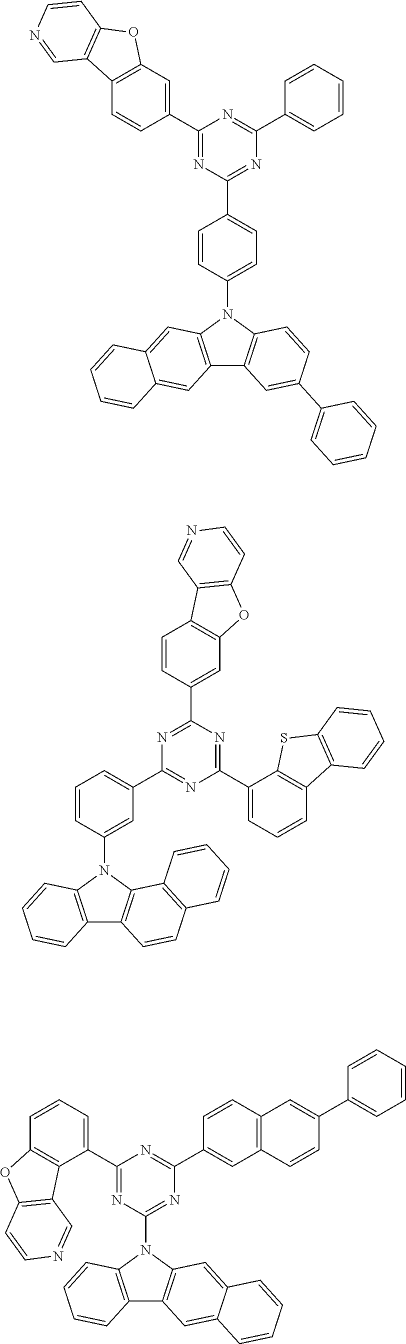 [SMILES: c1ccc(-c2ccc3c(c2)c2cc4ccccc4cc2n3-c2ccc(-c3nc(-c4ccccc4)nc(-c4ccc5c(c4)oc4ccncc45)n3)cc2)cc1 ; c1cc(-c2nc(-c3ccc4c(c3)oc3ccncc34)nc(-c3cccc4c3sc3ccccc34)n2)cc(-n2c3ccccc3c3ccc4ccccc4c32)c1 ; c1ccc(-c2ccc3cc(-c4nc(-c5cccc6oc7ccncc7c56)nc(-n5c6ccccc6c6cc7ccccc7cc65)n4)ccc3c2)cc1]

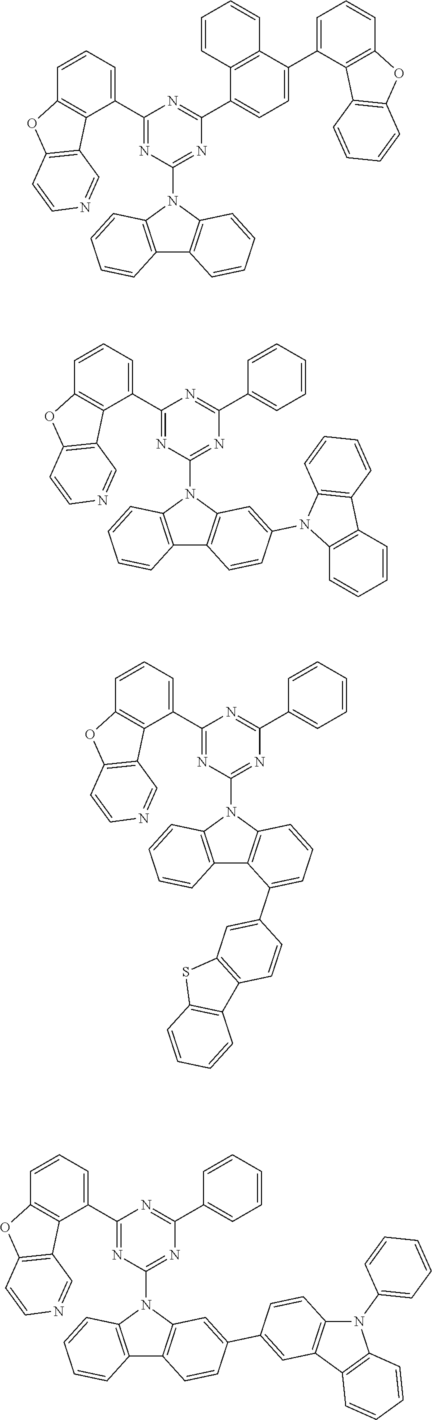 [SMILES: c1ccc2c(c1)oc1cccc(-c3ccc(-c4nc(-c5cccc6oc7ccncc7c56)nc(-n5c6ccccc6c6ccccc65)n4)c4ccccc34)c12 ; c1ccc(-c2nc(-c3cccc4oc5ccncc5c34)nc(-n3c4ccccc4c4ccc(-n5c6ccccc6c6ccccc65)cc43)n2)cc1 ; c1ccc(-c2nc(-c3cccc4oc5ccncc5c34)nc(-n3c4ccccc4c4c(-c5ccc6c(c5)sc5ccccc56)cccc43)n2)cc1 ; c1ccc(-c2nc(-c3cccc4oc5ccncc5c34)nc(-n3c4ccccc4c4ccc(-c5ccc6c(c5)c5ccccc5n6-c5ccccc5)cc43)n2)cc1]

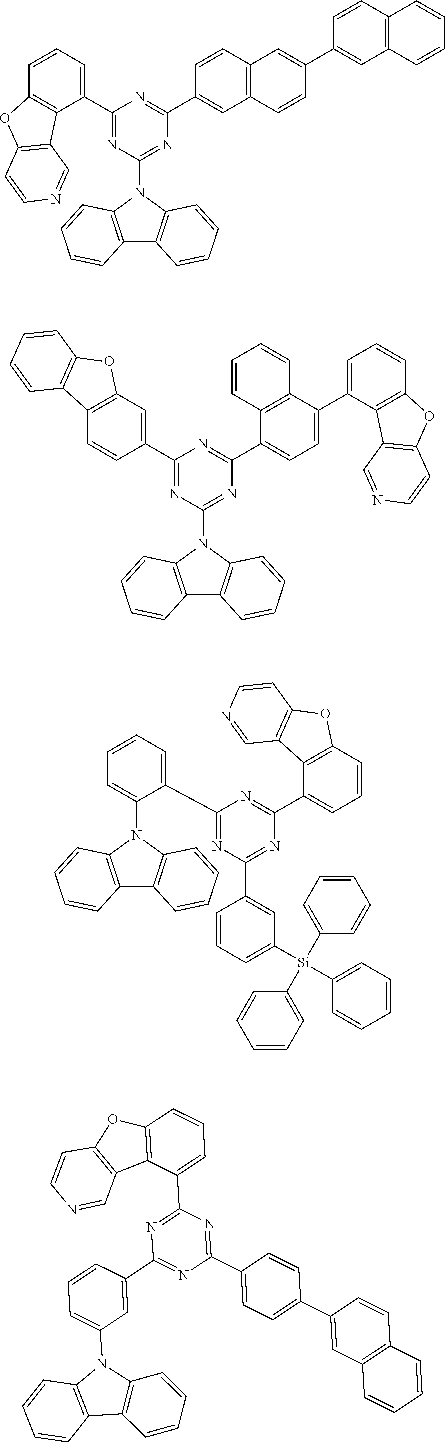 [SMILES: c1ccc2cc(-c3ccc4cc(-c5nc(-c6cccc7oc8ccncc8c67)nc(-n6c7ccccc7c7ccccc76)n5)ccc4c3)ccc2c1 ; c1ccc2c(c1)oc1cc(-c3nc(-c4ccc(-c5cccc6oc7ccncc7c56)c5ccccc45)nc(-n4c5ccccc5c5ccccc54)n3)ccc12 ; c1ccc([Si](c2ccccc2)(c2ccccc2)c2cccc(-c3nc(-c4ccccc4-n4c5ccccc5c5ccccc54)nc(-c4cccc5oc6ccncc6c45)n3)c2)cc1 ; c1cc(-c2nc(-c3ccc(-c4ccc5ccccc5c4)cc3)nc(-c3cccc4oc5ccncc5c34)n2)cc(-n2c3ccccc3c3ccccc32)c1]

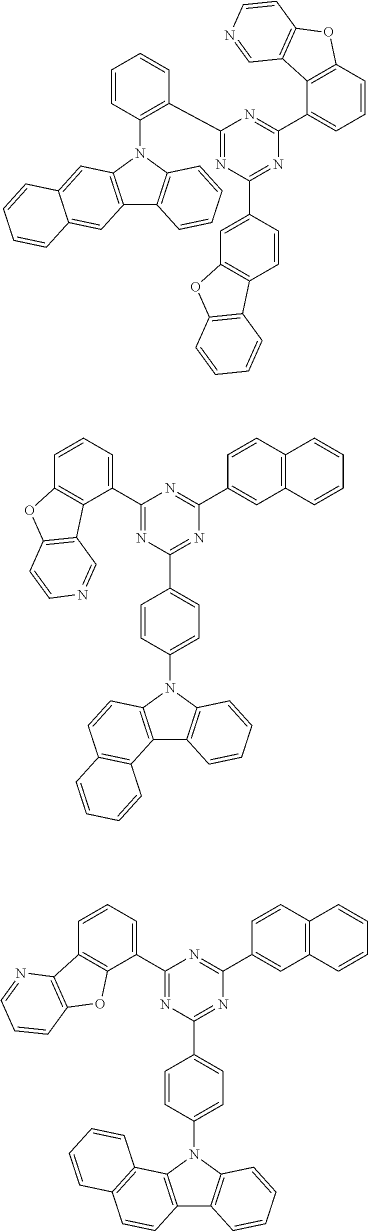 [SMILES: c1ccc(-n2c3ccccc3c3cc4ccccc4cc32)c(-c2nc(-c3ccc4c(c3)oc3ccccc34)nc(-c3cccc4oc5ccncc5c34)n2)c1 ; c1ccc2cc(-c3nc(-c4ccc(-n5c6ccccc6c6c7ccccc7ccc65)cc4)nc(-c4cccc5oc6ccncc6c45)n3)ccc2c1 ; c1ccc2cc(-c3nc(-c4ccc(-n5c6ccccc6c6ccc7ccccc7c65)cc4)nc(-c4cccc5c4oc4cccnc45)n3)ccc2c1]

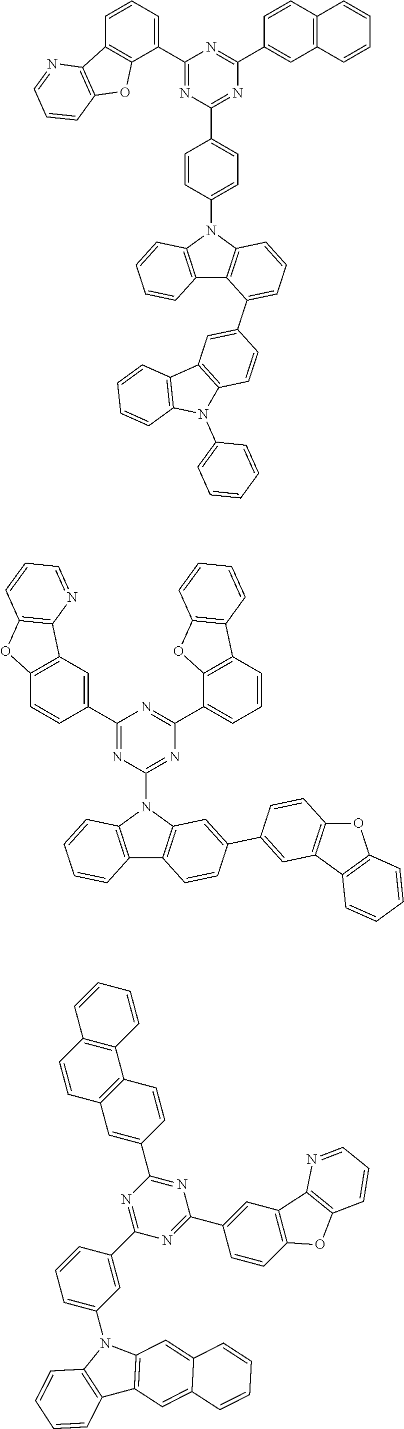 [SMILES: c1ccc(-n2c3ccccc3c3cc(-c4cccc5c4c4ccccc4n5-c4ccc(-c5nc(-c6ccc7ccccc7c6)nc(-c6cccc7c6oc6cccnc67)n5)cc4)ccc32)cc1 ; c1ccc2c(c1)oc1ccc(-c3ccc4c5ccccc5n(-c5nc(-c6ccc7oc8cccnc8c7c6)nc(-c6cccc7c6oc6ccccc67)n5)c4c3)cc12 ; c1cc(-c2nc(-c3ccc4c(ccc5ccccc54)c3)nc(-c3ccc4oc5cccnc5c4c3)n2)cc(-n2c3ccccc3c3cc4ccccc4cc32)c1]

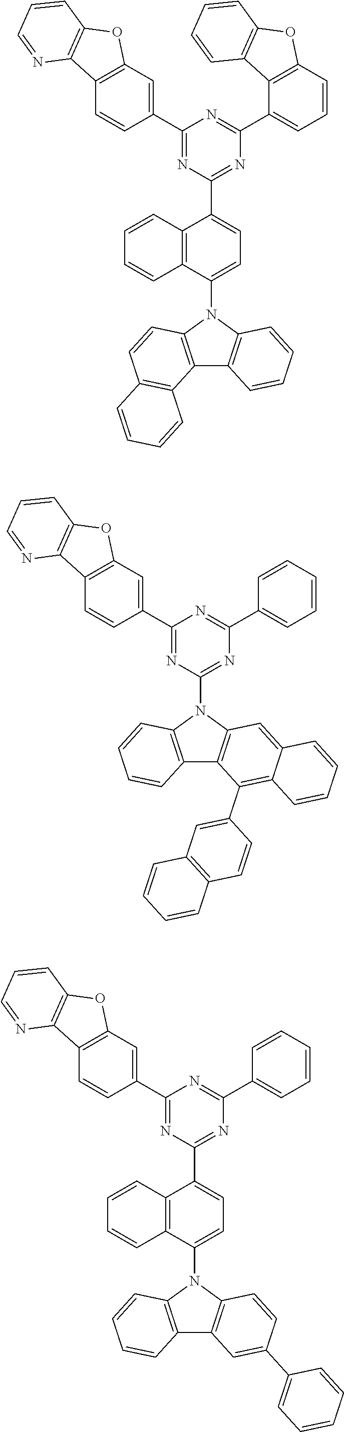 [SMILES: c1ccc2c(c1)ccc1c2c2ccccc2n1-c1ccc(-c2nc(-c3ccc4c(c3)oc3cccnc34)nc(-c3cccc4oc5ccccc5c34)n2)c2ccccc12 ; c1ccc(-c2nc(-c3ccc4c(c3)oc3cccnc34)nc(-n3c4ccccc4c4c(-c5ccc6ccccc6c5)c5ccccc5cc43)n2)cc1 ; c1ccc(-c2ccc3c(c2)c2ccccc2n3-c2ccc(-c3nc(-c4ccccc4)nc(-c4ccc5c(c4)oc4cccnc45)n3)c3ccccc23)cc1]

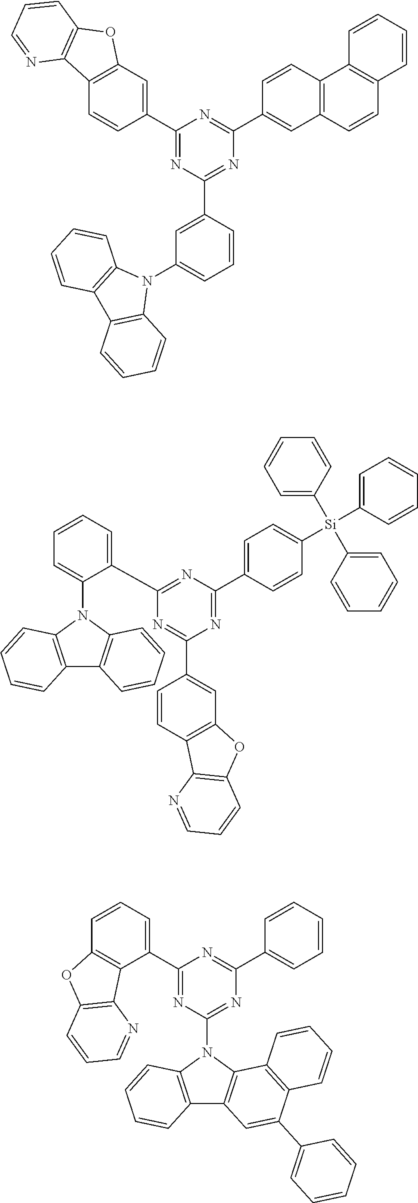 [SMILES: c1cc(-c2nc(-c3ccc4c(ccc5ccccc54)c3)nc(-c3ccc4c(c3)oc3cccnc34)n2)cc(-n2c3ccccc3c3ccccc32)c1 ; c1ccc([Si](c2ccccc2)(c2ccccc2)c2ccc(-c3nc(-c4ccc5c(c4)oc4cccnc45)nc(-c4ccccc4-n4c5ccccc5c5ccccc54)n3)cc2)cc1 ; c1ccc(-c2nc(-c3cccc4oc5cccnc5c34)nc(-n3c4ccccc4c4cc(-c5ccccc5)c5ccccc5c43)n2)cc1]

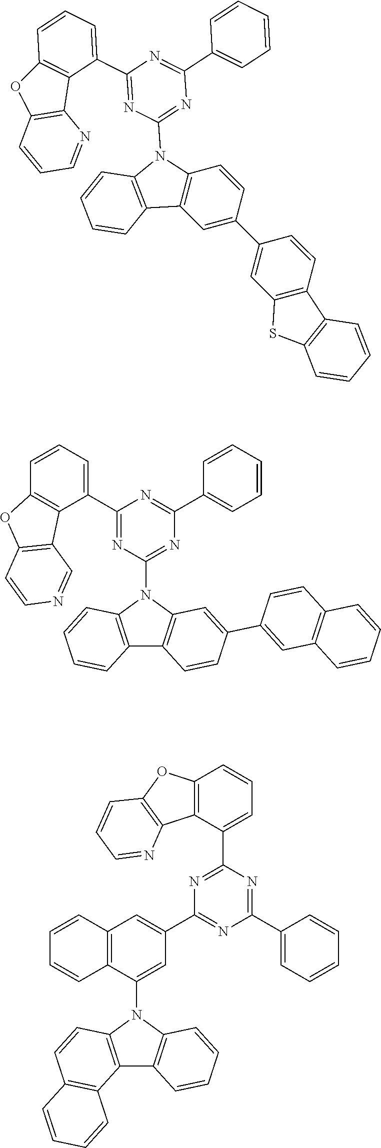 [SMILES: c1ccc(-c2nc(-c3cccc4oc5cccnc5c34)nc(-n3c4ccccc4c4cc(-c5ccc6c(c5)sc5ccccc56)ccc43)n2)cc1 ; c1ccc(-c2nc(-c3cccc4oc5ccncc5c34)nc(-n3c4ccccc4c4ccc(-c5ccc6ccccc6c5)cc43)n2)cc1 ; c1ccc(-c2nc(-c3cc(-n4c5ccccc5c5c6ccccc6ccc54)c4ccccc4c3)nc(-c3cccc4oc5cccnc5c34)n2)cc1]

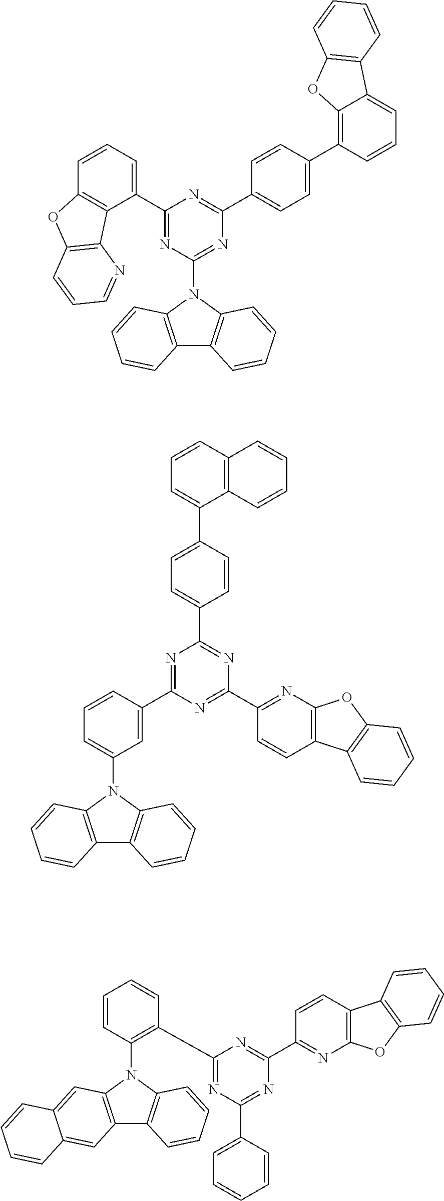 [SMILES: c1ccc2c(c1)oc1c(-c3ccc(-c4nc(-c5cccc6oc7cccnc7c56)nc(-n5c6ccccc6c6ccccc65)n4)cc3)cccc12 ; c1cc(-c2nc(-c3ccc(-c4cccc5ccccc45)cc3)nc(-c3ccc4c(n3)oc3ccccc34)n2)cc(-n2c3ccccc3c3ccccc32)c1 ; c1ccc(-c2nc(-c3ccc4c(n3)oc3ccccc34)nc(-c3ccccc3-n3c4ccccc4c4cc5ccccc5cc43)n2)cc1]

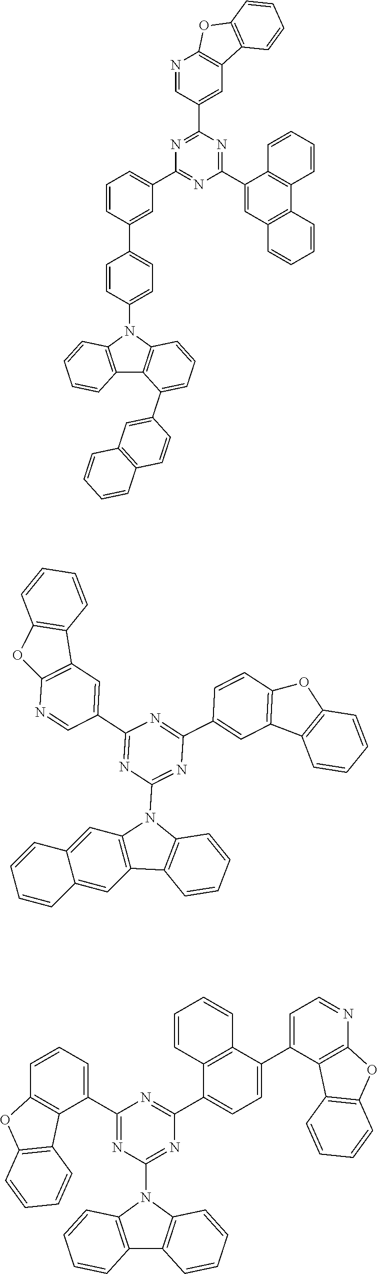 [SMILES: c1cc(-c2ccc(-n3c4ccccc4c4c(-c5ccc6ccccc6c5)cccc43)cc2)cc(-c2nc(-c3cnc4oc5ccccc5c4c3)nc(-c3cc4ccccc4c4ccccc34)n2)c1 ; c1ccc2cc3c(cc2c1)c1ccccc1n3-c1nc(-c2ccc3oc4ccccc4c3c2)nc(-c2cnc3oc4ccccc4c3c2)n1 ; c1ccc2c(c1)oc1cccc(-c3nc(-c4ccc(-c5ccnc6oc7ccccc7c56)c5ccccc45)nc(-n4c5ccccc5c5ccccc54)n3)c12]

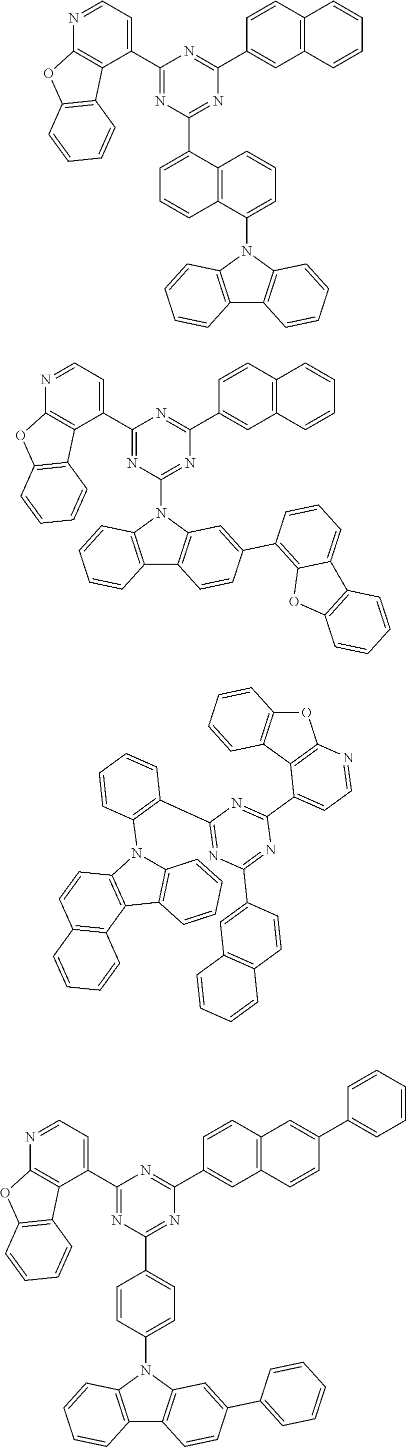

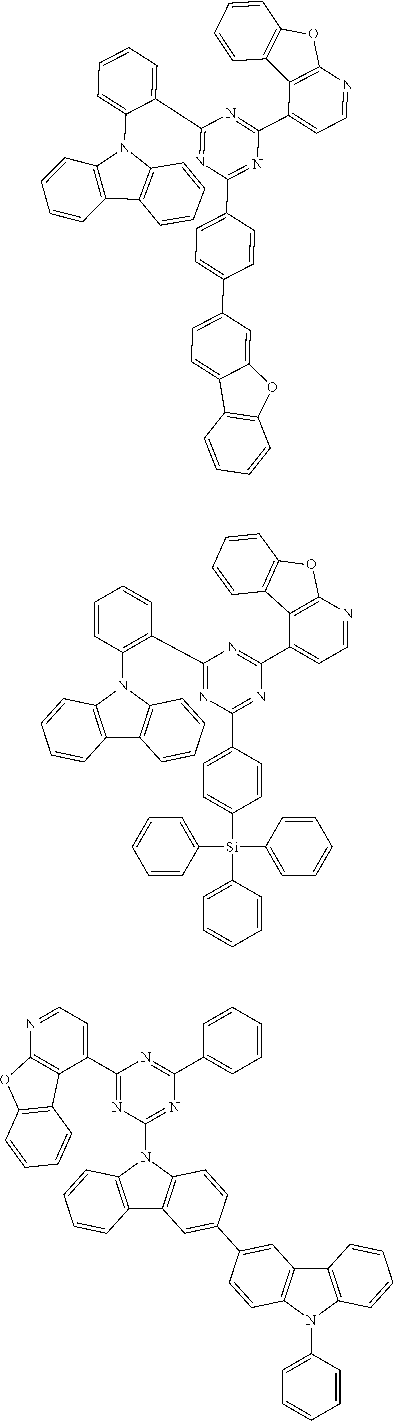 [SMILES: c1ccc(-n2c3ccccc3c3ccccc32)c(-c2nc(-c3ccc(-c4ccc5c(c4)oc4ccccc45)cc3)nc(-c3ccnc4oc5ccccc5c34)n2)c1 ; c1ccc([Si](c2ccccc2)(c2ccccc2)c2ccc(-c3nc(-c4ccccc4-n4c5ccccc5c5ccccc54)nc(-c4ccnc5oc6ccccc6c45)n3)cc2)cc1 ; c1ccc(-c2nc(-c3ccnc4oc5ccccc5c34)nc(-n3c4ccccc4c4cc(-c5ccc6c(c5)c5ccccc5n6-c5ccccc5)ccc43)n2)cc1]

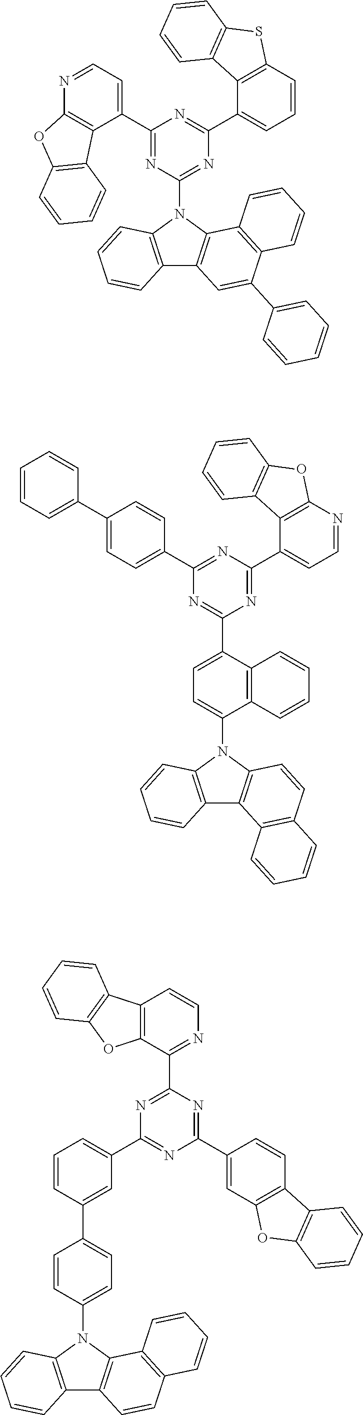 [SMILES: c1ccc(-c2cc3c4ccccc4n(-c4nc(-c5ccnc6oc7ccccc7c56)nc(-c5cccc6sc7ccccc7c56)n4)c3c3ccccc23)cc1 ; c1ccc(-c2ccc(-c3nc(-c4ccc(-n5c6ccccc6c6c7ccccc7ccc65)c5ccccc45)nc(-c4ccnc5oc6ccccc6c45)n3)cc2)cc1 ; c1cc(-c2ccc(-n3c4ccccc4c4ccc5ccccc5c43)cc2)cc(-c2nc(-c3ccc4c(c3)oc3ccccc34)nc(-c3nccc4c3oc3ccccc34)n2)c1]

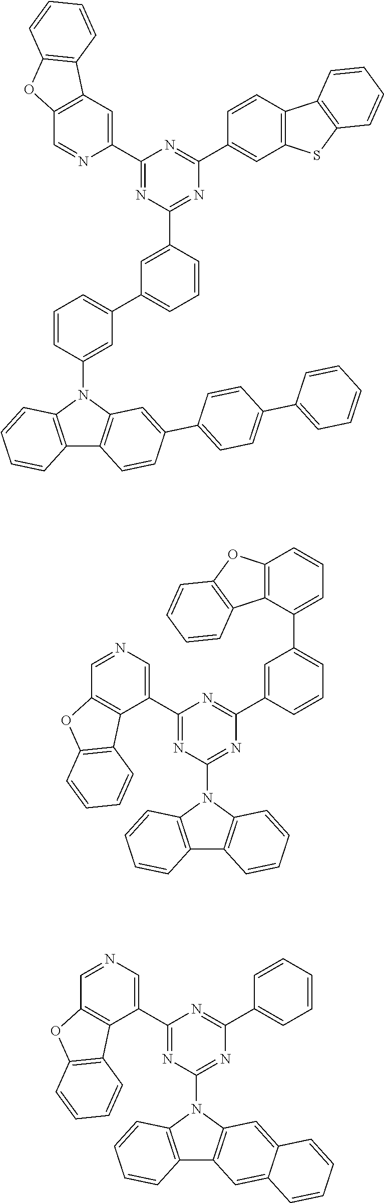 [SMILES: c1ccc(-c2ccc(-c3ccc4c5ccccc5n(-c5cccc(-c6cccc(-c7nc(-c8ccc9c(c8)sc8ccccc89)nc(-c8cc9c(cn8)oc8ccccc89)n7)c6)c5)c4c3)cc2)cc1 ; c1cc(-c2nc(-c3cncc4oc5ccccc5c34)nc(-n3c4ccccc4c4ccccc43)n2)cc(-c2cccc3oc4ccccc4c23)c1 ; c1ccc(-c2nc(-c3cncc4oc5ccccc5c34)nc(-n3c4ccccc4c4cc5ccccc5cc43)n2)cc1]

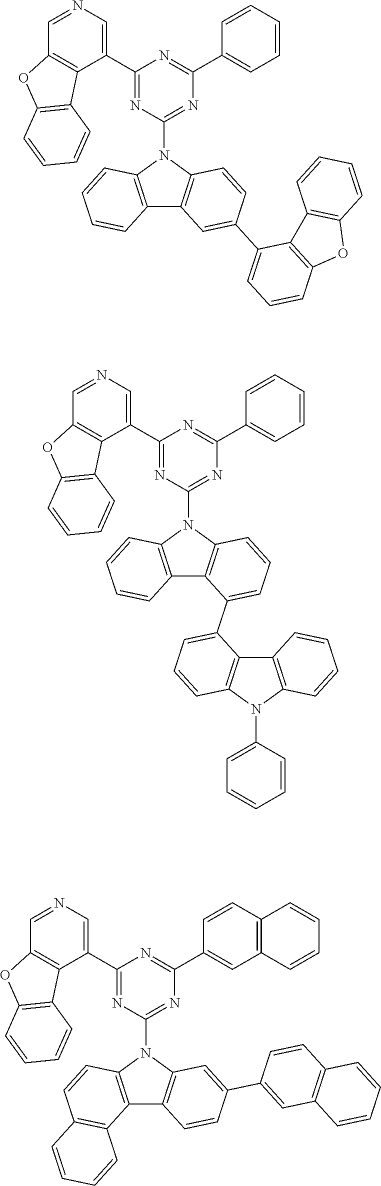 [SMILES: c1ccc(-c2nc(-c3cncc4oc5ccccc5c34)nc(-n3c4ccccc4c4cc(-c5cccc6oc7ccccc7c56)ccc43)n2)cc1 ; c1ccc(-c2nc(-c3cncc4oc5ccccc5c34)nc(-n3c4ccccc4c4c(-c5cccc6c5c5ccccc5n6-c5ccccc5)cccc43)n2)cc1 ; c1ccc2cc(-c3ccc4c5c6ccccc6ccc5n(-c5nc(-c6ccc7ccccc7c6)nc(-c6cncc7oc8ccccc8c67)n5)c4c3)ccc2c1]

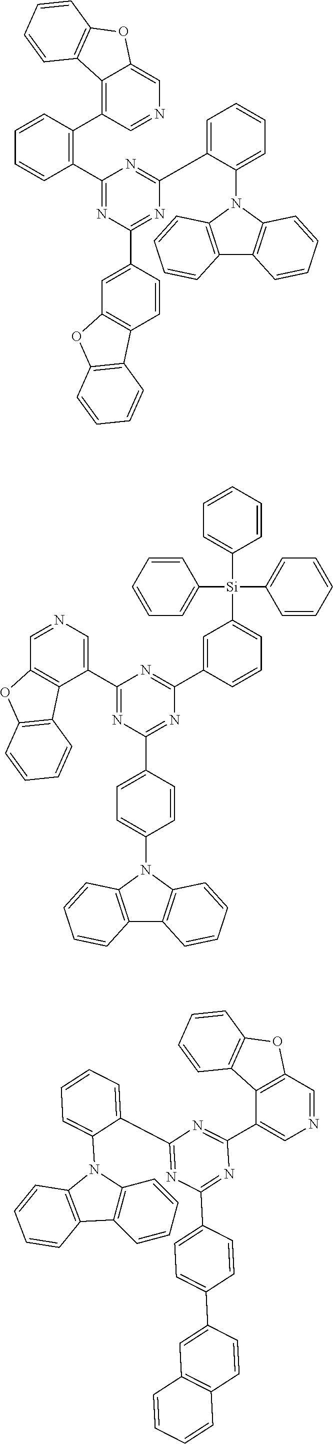 [SMILES: c1ccc(-c2cncc3oc4ccccc4c23)c(-c2nc(-c3ccc4c(c3)oc3ccccc34)nc(-c3ccccc3-n3c4ccccc4c4ccccc43)n2)c1 ; c1ccc([Si](c2ccccc2)(c2ccccc2)c2cccc(-c3nc(-c4ccc(-n5c6ccccc6c6ccccc65)cc4)nc(-c4cncc5oc6ccccc6c45)n3)c2)cc1 ; c1ccc(-n2c3ccccc3c3ccccc32)c(-c2nc(-c3ccc(-c4ccc5ccccc5c4)cc3)nc(-c3cncc4oc5ccccc5c34)n2)c1]

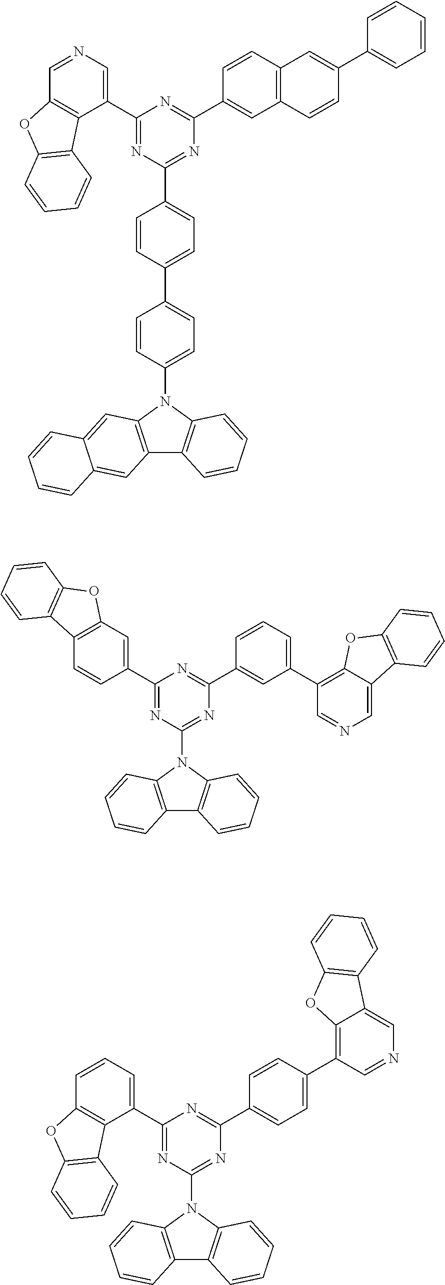 [SMILES: c1ccc(-c2ccc3cc(-c4nc(-c5ccc(-c6ccc(-n7c8ccccc8c8cc9ccccc9cc87)cc6)cc5)nc(-c5cncc6oc7ccccc7c56)n4)ccc3c2)cc1 ; c1cc(-c2nc(-c3ccc4c(c3)oc3ccccc34)nc(-n3c4ccccc4c4ccccc43)n2)cc(-c2cncc3c2oc2ccccc23)c1 ; c1ccc2c(c1)oc1c(-c3ccc(-c4nc(-c5cccc6oc7ccccc7c56)nc(-n5c6ccccc6c6ccccc65)n4)cc3)cncc12]

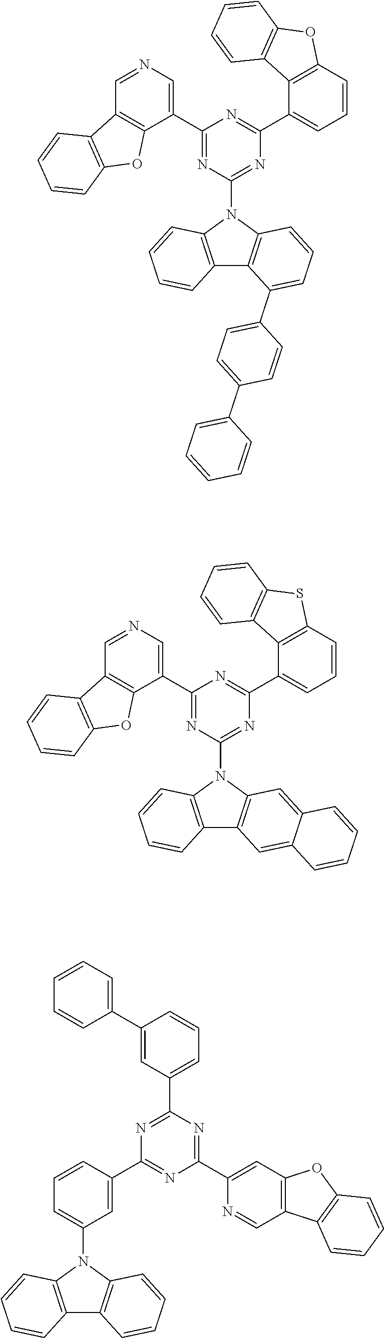 [SMILES: c1ccc(-c2ccc(-c3cccc4c3c3ccccc3n4-c3nc(-c4cncc5c4oc4ccccc45)nc(-c4cccc5oc6ccccc6c45)n3)cc2)cc1 ; c1ccc2cc3c(cc2c1)c1ccccc1n3-c1nc(-c2cncc3c2oc2ccccc23)nc(-c2cccc3sc4ccccc4c23)n1 ; c1ccc(-c2cccc(-c3nc(-c4cccc(-n5c6ccccc6c6ccccc65)c4)nc(-c4cc5oc6ccccc6c5cn4)n3)c2)cc1]

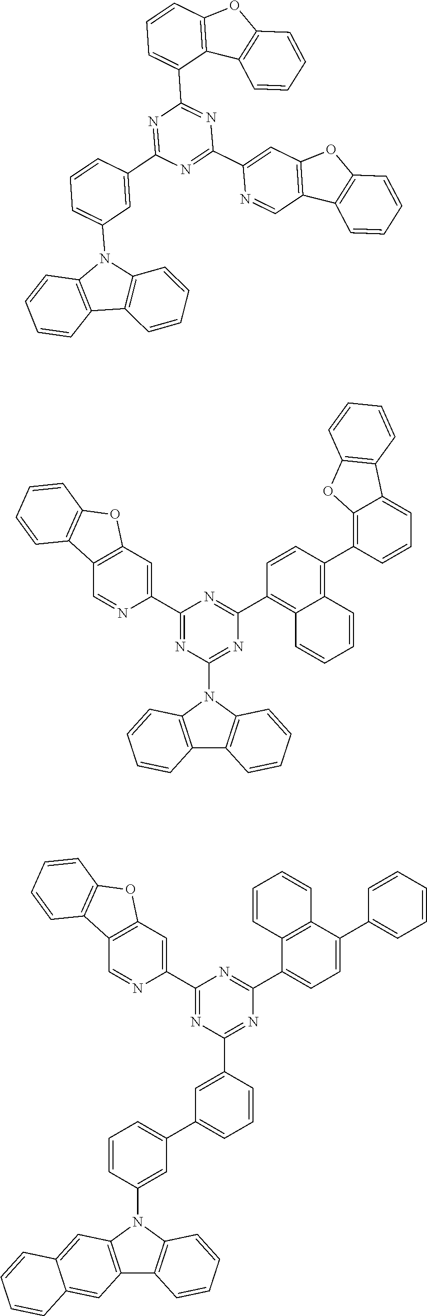 [SMILES: c1cc(-c2nc(-c3cc4oc5ccccc5c4cn3)nc(-c3cccc4oc5ccccc5c34)n2)cc(-n2c3ccccc3c3ccccc32)c1 ; c1ccc2c(c1)oc1cc(-c3nc(-c4ccc(-c5cccc6c5oc5ccccc56)c5ccccc45)nc(-n4c5ccccc5c5ccccc54)n3)ncc12 ; c1ccc(-c2ccc(-c3nc(-c4cccc(-c5cccc(-n6c7ccccc7c7cc8ccccc8cc76)c5)c4)nc(-c4cc5oc6ccccc6c5cn4)n3)c3ccccc23)cc1]

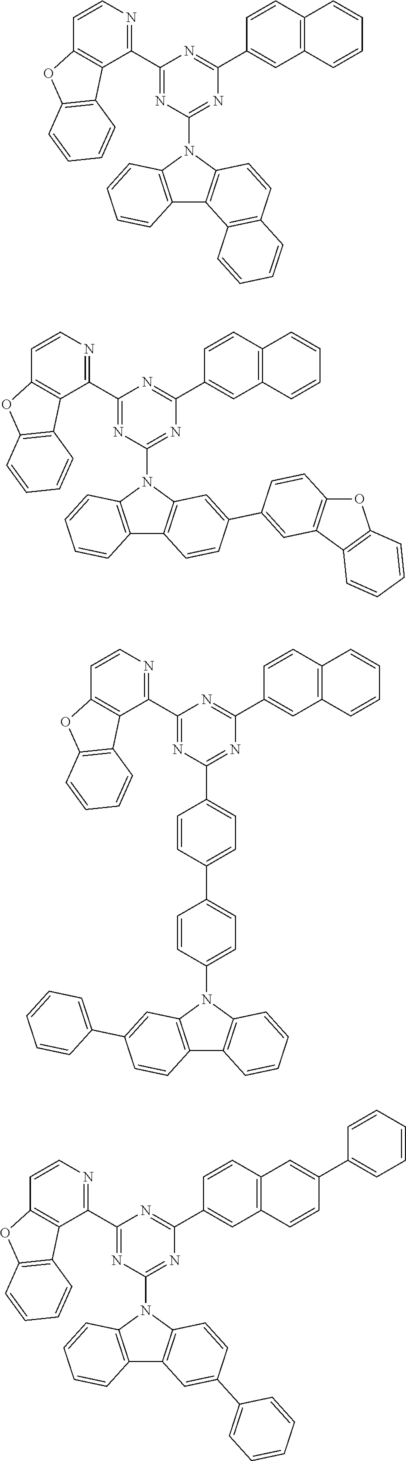 [SMILES: c1ccc2cc(-c3nc(-c4nccc5oc6ccccc6c45)nc(-n4c5ccccc5c5c6ccccc6ccc54)n3)ccc2c1 ; c1ccc2cc(-c3nc(-c4nccc5oc6ccccc6c45)nc(-n4c5ccccc5c5ccc(-c6ccc7oc8ccccc8c7c6)cc54)n3)ccc2c1 ; c1ccc(-c2ccc3c4ccccc4n(-c4ccc(-c5ccc(-c6nc(-c7ccc8ccccc8c7)nc(-c7nccc8oc9ccccc9c78)n6)cc5)cc4)c3c2)cc1 ; c1ccc(-c2ccc3cc(-c4nc(-c5nccc6oc7ccccc7c56)nc(-n5c6ccccc6c6cc(-c7ccccc7)ccc65)n4)ccc3c2)cc1]

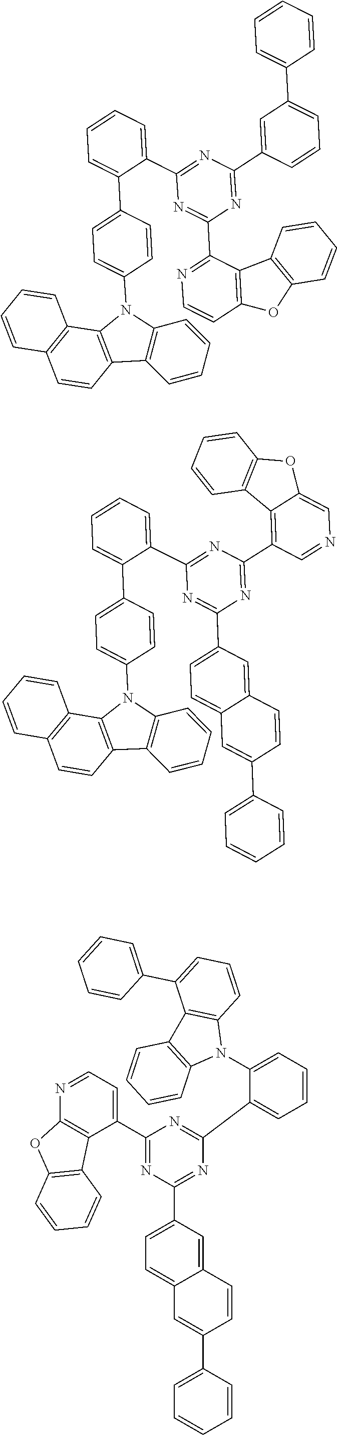 [SMILES: c1ccc(-c2cccc(-c3nc(-c4ccccc4-c4ccc(-n5c6ccccc6c6ccc7ccccc7c65)cc4)nc(-c4nccc5oc6ccccc6c45)n3)c2)cc1 ; c1ccc(-c2ccc3cc(-c4nc(-c5ccccc5-c5ccc(-n6c7ccccc7c7ccc8ccccc8c76)cc5)nc(-c5cncc6oc7ccccc7c56)n4)ccc3c2)cc1 ; c1ccc(-c2ccc3cc(-c4nc(-c5ccccc5-n5c6ccccc6c6c(-c7ccccc7)cccc65)nc(-c5ccnc6oc7ccccc7c56)n4)ccc3c2)cc1]

12. An organic light emitting device comprising:
a first electrode;
a second electrode provided to face the first electrode; and
an organic material layer including one or more layers provided between the first electrode and the second electrode,
wherein one or more layers of the organic material layer comprises the compound of claim 1.

13. The organic light emitting device of claim 12, wherein
the organic material layer comprises a light emitting layer, and the light emitting layer includes the compound.
